(19) United States
(12) Patent Application Publication
(10) Pub. No.: US 2005/0287551 A1
(43) Pub. Date: Dec. 29, 2005

(54) Susceptibility gene for human stroke; methods of treatment

(75) Inventors: Solveig Gretarsdottir, Reykjavik (IS); Gudmar Thorleifsson, Reykjavik (IS); Jeffrey R. Gulcher, Lake Barrington, IL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 11/091,018

(22) Filed: Mar. 25, 2005

Related U.S. Application Data
(63) Continuation of application No. PCT/US03/29906, filed on Sep. 25, 2003.
Application No. PCT/US03/29906 is a continuation of application No. 10/650,120, filed on Aug. 27, 2003, now abandoned.
Application No. PCT/US03/29906 is a continuation-in-part of application No. 10/419,723, filed on Apr. 18, 2003.
Application No. 10/419,723 is a continuation-in-part of application No. 10/255,120, filed on Sep. 25, 2002.
Application No. 10/255,120 is a continuation-in-part of application No. 10/067,514, filed on Feb. 4, 2002, now abandoned.
Application No. 10/067,514 is a continuation-in-part of application No. 09/811,352, filed on Mar. 19, 2001, now abandoned.

(51) Int. Cl. C12Q 1/68

(52) U.S. Cl. 435/6

(57) ABSTRACT

A role of the human PDE4D gene in stroke is disclosed. Methods for diagnosis, prediction of clinical course and treatment for stroke using polymorphisms in the PDE4D gene are also disclosed.

### RELATED APPLICATIONS

This application is a continuation of International Application No. PCT/US03/29906, which designated the United States and was filed on Sep. 25, 2003, published in English, which is a continuation of and claims priority to U.S. application Ser. No. 10/650,120, filed Aug. 27, 2003, which is a continuation-in-part of U.S. application Ser. No. 10/419,723 filed Apr. 18, 2003, which is a continuation-in-part of U.S. application Ser. No. 10/255,120, filed Sep. 25, 2002, which is a continuation-in-part of U.S. application Ser. No. 10/067,514, filed Feb. 4, 2002, which is a continuation-in-part of U.S. application Ser. No. 09/811,352, filed Mar. 19, 2001. The entire teachings of the above applications are incorporated herein by reference.

### BACKGROUND OF THE INVENTION

Stroke is a common and serious disease. Each year in the United States more than 600,000 individuals suffer a stroke and more than 160,000 die from stroke-related causes (Sacco, R. L. et al., Stroke 28, 1507-17 (1997)). In western countries stroke is the leading cause of severe disability and the third leading cause of death (Bonita, R., Lancet 339, 342-4 (1992)). The lifetime risk of those who reach the age of 40 exceeds 10%.

The clinical phenotype of stroke is complex but is broadly divided into ischemic (accounting for 80-90%) and hemorrhagic stroke (10-20%) (Caplan, L. R. Caplan's Stroke: A Clinical Approach, 1-556 (Butterworth-Heinemann, 2000)). Ischemic stroke is further subdivided into large vessel occlusive disease (referred to here as carotid stroke), usually due to atherosclerotic involvement of the common and internal carotid arteries, small vessel occlusive disease, thought to be a non-atherosclerotic narrowing of small end-arteries within the brain, and cardiogenic stroke due to blood clots arising from the heart usually on the background of atrial fibrillation or ischemic (atherosclerotic) heart disease (Adams, H. P., Jr. et al., Stroke 24, 35-41 (1993)). Therefore, it appears that stroke is not one disease but a heterogeneous group of disorders reflecting differences in the pathogenic mechanisms (Alberts, M. J. Genetics of Cerebrovascular Disease, 386 (Futura Publishing Company, Inc., New York, 1999); Hassan, A. & Markus, H. S. Brain 123, 1784-812 (2000)). However, all forms of stroke share risk factors such as hypertension, diabetes, hyperlipidemia, and smoking (Sacco, R. L. et al., Stroke 28, 1507-17 (1997); Leys, D. et al., J. Neurol. 249, 507-17 (2002)). Family history of stroke is also an independent risk factor suggesting the existence of genetic factors that may interact with environmental factors (Hassan, A. & Markus, H. S. Brain 123, 1784-812 (2000); Brass, L. M. & Alberts, M. J. Baillieres Clin. Neurol. 4, 221-45 (1995)).

The genetic determinants of the common forms of stroke are still largely unknown. There are examples of mutations in specific genes that cause rare Mendelian forms of stroke such as the Notch3 gene in CADASIL (cerebral autosomal dominant arteriopathy with subcortical infarctions and leukoencephalopathy) (Tournier-Lasserve, E. et al., Nat. Genet. 3, 256-9 (1993); Joutel, A. et al., Nature 383, 707-10 (1996)), Cystatin C in the Icelandic type of hereditary cerebral hemorrhage with amyloidosis (Palsdottir, A. et al., Lancet 2, 603-4 (1988)), APP in the Dutch type of hereditary cerebral hemorrhage (Levy, E. et al., Science 248, 1124-6 (1990)) and the KRIT1 gene in patients with hereditary cavernous angioma (Gunel, M. et al., Proc. Natl. Acad. Sci. USA 92, 6620-4 (1995); Sahoo, T. et al., Hum. Mol. Genet. 8, 2325-33 (1999)). None of these rare forms of stroke occur on the background of atherosclerosis, and therefore, the corresponding genes are not likely to play roles in the common forms of stroke which most often occur with atherosclerosis.

It is very important for the health care system to develop strategies to prevent stroke. Once a stroke happens, irreversible cell death occurs in a significant portion of the brain supplied by the blood vessel affected by the stroke. Unfortunately, the neurons that die cannot be revived or replaced from a stem cell population. Therefore, there is a need to prevent strokes from happening in the first place. Although we already know of certain clinical risk factors that increase stroke risk (listed above), there is an unmet medical need to define the genetic factors involved in stroke to more precisely define stroke risk. Further, if predisposing alleles are common in the general population and the specificity of predicting a disease based on their presence is low, additional loci such as protective loci are needed for meaningful prediction of disposition of the disease state. There is also a great need for therapeutic agents for preventing the first stroke or further strokes in individuals who have suffered a previous stroke or transient ischemic attack.

### SUMMARY OF THE INVENTION

A locus conferring susceptibility to ischemic stroke to chromosome 5q12 in the Icelandic population has been mapped and the identification of phosphodiesterase 4D (PDE4D) as the gene at 5q12 contributing to the risk of ischemic stroke has been reported. This locus was extensively fine mapped and tested for association to stroke. Most striking is that haplotypes can be classified into three distinct groups: wild type, at-risk and protective. Additionally, a significant disregulation of multiple PDE4D isoforms in stroke patients was observed. The strongest association was within the PDE4D, especially to the two major subtypes of ischemic stroke, carotid and cardiogenic stroke. We have found variation in PDE4D that more than doubles the risk for cardiogenic and carotid stroke, two of the most common forms of ischemic stroke. We have shown that there are at least 9 isoforms of PDE4D at the mRNA level and the protein level. The basis for these isoforms is the use of alternative 5 prime exons that are alternatively spliced into a common set of exons defining the catalytic domain as well as, in the case of the long forms, a set of exons defining a common core in the regulatory domain. The PDE4D gene is involved in the pathogenesis of stroke. The PDE4D gene may be involved through artherosclerosis, the major pathological process underlying ischemic stroke. Our results indicate that atherosclerosis is a cAMP disease resulting from disregulation of its levels within the vasculature.

In one aspect, the invention relates to methods of diagnosing a predisposition to stroke. The methods of diagnosing a predisposition to stroke in an individual include detecting the presence of a polymorphism in PDE4D, as well as detecting alterations in expression of a PDE4D polypeptide or isoform, such as the presence of, or relative expression of different splicing variants of PDE4D polypeptides. For example, it may be that the ratio of certain splice variants could be used as a diagnostic marker for stroke predisposition. Also an abnormal splice form can be detected (that is one that is not normally expressed but is created from a DNA sequence mutation that leads to an abnormal splice form to be created from the primary transcript) may be created from mutations in the PDE4D gene. For example, new splice sites might be created from a single base substitution within an intron that is inappropriately used as a splice acceptor or donor site, resulting in an abnormal message which is likely to have a premature stop codon leading to a truncated form of PDE4D protein. The alterations in expression can be quantitative, qualitative, or both quantitative and qualitative. The methods of the invention allow the accurate diagnosis of stroke at or before disease onset, thus reducing or minimizing the debilitating effects of stroke. The methods of the invention also diagnose those individuals who are protected against developing stroke even in the face of other risk factors including but not restricted to hypertension, diabetes, hyperlipidemia, smoking history, previous stroke, TIA, MI or PAOD, or carriers of stroke associated gene variants. In one embodiment, predisposition to stroke or susceptibility to stroke can be assessed by determining PDE4D isoform levels in the individual compared to control levels, wherein a difference in isoform expression is indicative of predisposition or susceptibility to stroke. Preferably, the level of expression of PDE4D7 and/or PDE4D9 is assessed.

The invention additionally relates to an assay for identifying agents that alter (e.g., enhance or inhibit) the activity or expression or transcription of one or more PDE4D polypeptides or isoforms. Such an assay may also identify agents that alter the relative expression of one or more PDE4D isoforms with respect to other isoforms at either the mRNA level or polypeptide level. For example, a cell, cellular fraction, or solution containing a PDE4D polypeptide or a fragment or derivative thereof, can be contacted with an agent to be tested, and the level of PDE4D polypeptide expression or activity can be assessed. Alternatively, a cell, or cell with artificial DNA construct with part or all of the PDE4D gene with or without a reporter gene can be used to identify agents that may directly affect transcription at one or more of the many alternative PDE4D promoters upstream of the alternative 5 prime exons or splicing efficiency of the primary transcript to one or more mRNA isoforms. The activity or expression of more than one PDE4D polypeptides can be assessed concurrently (or the corresponding reporter gene activity) (e.g., the cell, cellular fraction, or solution can contain more than one type of PDE4D polypeptide, such as different splicing variants, and the levels of the different polypeptides or splicing mRNA variants can be assessed).

Agents that enhance or inhibit PDE4D mRNA or polypeptide expression or activity are also included in the current invention, as are methods of altering (enhancing or inhibiting) PDE4D mRNA or polypeptide expression or activity by contacting a cell containing PDE4D gene, mRNA, and/or polypeptide, or by contacting the PDE4D gene, mRNA, and/or polypeptide, with an agent that enhances or inhibits expression or activity of PDE4D mRNA or polypeptide. In another embodiment, isoform mRNA and/or protein levels can be altered, compared to control levels, using the agents of the invention.

Additionally, the invention pertains to pharmaceutical compositions comprising the nucleic acids of the invention, the polypeptides of the invention, and/or the agents that alter activity of PDE4D polypeptide. The invention further pertains to methods of treating stroke, by administering PDE4D therapeutic agents, such as nucleic acids of the invention, polypeptides of the invention, the agents that alter activity of PDE4D polypeptide, or compositions comprising the nucleic acids, polypeptides, and/or the agents that alter activity of PDE4D polypeptide.

The invention further relates to methods for preventing the occurrence of stroke in an individual in need thereof by regulating a PDE4D mRNA and/or polypeptide isoform level compared to control levels, whereby the regulated isoform level mimics the level of a healthy individual. Isoform expression at the mRNA and/or polypeptide level can be regulated using the agents and pharmaceutical compositions of the invention, by genetic alteration, by altering the ratio of isoforms and/or their absolute expression. In one embodiment, isoforms PDE4D7 and/or PDE4D9 can be regulated.

The invention further provides a method of diagnosing susceptibility to stroke in an individual. This method comprises screening for one of the at-risk haplotypes in the phosphodiesterase 4D gene that is more frequently present in an individual susceptible to stroke, compared to the frequency of its presence in the general population, wherein the presence of an at-risk haplotype is indicative of a susceptibility to stroke. An “at-risk haplotype” is intended to embrace one or a combination of haplotypes described herein over the PDE4D gene that show high correlation to stroke. In one embodiment, the at-risk haplotype is characterized by the presence of at least one single nucleotide polymorphism at nucleic acid positions at risk haplotype 1 is G at nucleic acid position 142780 respectively, relative to SEQ ID NO: 1 and allele 0 of microsatellite marker AC0088181-1. In another embodiment, the at-risk haplotype 2 is characterized by the presence of at least one single nucleotide polymorphism and microsatellite marker at nucleic acid positions 142780, 135112, 132562, 131865, 129361, 129360, 125304, 123426, 123312, 120628, 118914, 111781, 111252, 109301, 107849, 105225, 104552, 102977, 100795, 99035, 88614, 88456, 83119, 82244, 80127, 78552, relative to SEQ ID NO: 1 and allele 0 microsatellite marker AC0088181-1.

In yet another embodiment, the at-risk haplotype 3 is characterized by the presence of at least one polymorphism at nucleic acid positions 138806, 131865, 129361, 120628, 91470 relative to SEQ ID NO: 1.

Also described are methods for diagnosing susceptibility to stroke in an individual comprising screening for an at-risk haplotype in the phosphodiesterase 4D gene that is more frequently present in an individual susceptible to stroke (affected), compared to the frequency of its presence in a healthy individual (control) wherein the screening for the presence of an at-risk haplotype within or near PDE4D that significantly correlates with at least one of the haplotypes described herein or stroke susceptibility. As an example of a simple test for correlation would be a Fisher-exact test on a two by two table. Given a cohort of chromosomes the two by two table is constructed out of the number of chromosomes that include both of the haplotypes, one of the haplotype but not the other and neither of the haplotypes.

A protective haplotype is intended to embrace one or a combination of haplotypes described herein over the PDE4D gene that show a protective characteristic or property of a reduced risk of stroke. The particular combination of genetic markers (haplotypes) are present at a higher than expected frequency in controls than patients. Individuals with a protective allele or haplotype are about 30% less likely to have a stroke compared to the general population. In one embodiment, a protective haplotype is characterized by the presence of at least one single nucleotide polymorphism, such as the allele A at nucleotide position 142780 relative to SEQ ID NO: 1. The presence of the polymorphisms that comprise the at-risk haplotype or protective haplotype can be determined by electrophoretic analysis, restriction length polymorphism analysis, fluorescence energy transfer detection, kinetic PCR, allele specific PCR, sequence analysis, hybridization analysis or other known techniques.

Kits for diagnosing susceptibility to stroke in an individual are also disclosed and comprise primers for nucleic acid amplification of a region of PDE4D comprising the at-risk haplotype and/or protective haplotype.

The first major application of the current invention involves prediction of those at higher risk of developing a stroke. Diagnostic tests that define genetic factors contributing to stroke might be used together with or independent of the known clinical risk factors to define an individual's risk relative to the general population. Better means for identifying those individuals at risk for stroke should lead to better prophylactic and treatment regimens, including more aggressive management of the current clinical risk factors such as hypertension, diabetes, hypercholesterolemia, hypertriglyceridemia, obesity, and inflammatory components as reflected by increased C-reactive protein levels or other inflammatory markers. Information on genetic risk may be used by physicians to help convince particular patients to adjust life style and quit smoking. This invention provides the means to define a genetic component that doubles an individual's risk for stroke. Also described are means to define the genetic components that protect an individual from stroke.

The second major application of the current invention is the specific identification of a rate-limiting pathway involved in stroke. While many have attempted to find genes that are over-expressed or under-expressed in atherosclerosis plaques in the carotid arteries, the vast majority of the changes seen in diseased blood vessels compared to normal blood vessels are simply a reaction to the underlying process of atherosclerosis and stroke predisposition and are not the underlying cause. A disease gene with genetic variation that is significantly more common in stroke patients as compared to controls represents a specifically validated causative step in the pathogenesis of stroke. That is, the uncertainty about whether a gene is causative or simply reactive to the disease process is eliminated. The protein encoded by the disease gene defines a rate-limiting molecular pathway involved in the biological process of stroke predisposition. The proteins encoded by such stroke genes or its interacting proteins in its molecular pathway may represent drug targets that may be selectively modulated by small molecule, protein, antibody, or nucleic acid therapies. Such specific information is greatly needed since stroke prevention and treatment is a major unmet medical need that affects over a half-million Americans each year. Also useful is determining the gene that is protective against stroke. The proteins encoded by the protective gene and the biological pathway that it is a member may represent another target selectively modulated by small molecule, protein antibody or nucleic acid therapies.

A third application of the current invention is its use to predict an individual's response to a particular drug, even drugs that do not act on PDE4D or its pathway. It is a well-known phenomenon that in general, patients do not respond equally to the same drug. Much of the differences in drug response to a given drug is thought to be based on genetic and protein differences among individuals in certain genes and their corresponding pathways. Our invention defines the PDE4D pathway and its effect on cAMP levels in cells where it is expressed as one key molecular pathway involved in stroke risk. Some current or future therapeutic agents may be able to affect this pathway directly or indirectly and therefore, be effective in those patients whose stroke risk is in part determined by PDE4D pathway genetic variation. On the other hand, those same drugs may be less effective or ineffective in those patients who do not have at risk variation in the PDE4D gene or pathway. Therefore, PDE4D variation or haplotypes may be used as a pharmacogenomic diagnostic to predict drug response and guide choice of therapeutic agent in a given individual.

The invention helps meet the unmet medical needs in at least two major ways: 1) it provides a means to define patients at higher risk for stroke than the general population who can be more aggressively managed by their physicians in an effort to prevent stroke; and 2) it defines a drug target that can be used to screen and develop therapeutic agents that can be used to prevent stroke before it happens or prevent a second stroke in those who have already suffered a stroke or transient ischemic attack.

### BRIEF DESCRIPTION OF THE DRAWINGS

The foregoing and other objects, features and advantages of the invention will be apparent from the following more particular description of certain embodiments of the invention, as illustrated in the accompanying drawings.

FIGS. 1.1 and 1.2 show two family pedigrees each affected by several of the stroke subtypes, including hemorrhagic stroke.

FIGS. 2.1, 2.2 and 2.3 show the genetic, combined and physical maps for locating the PDE4D gene using 30 polymorphic markers. For the combined map, all markers have been assigned in the genetic and physical map unless otherwise indicated (* indicates marker only assigned in the physical map; ** indicates markers only assigned in genetic map).

FIG. 3 shows the schematic representations of PDE4D splice variants. Splice variants PDE4D9 are novel, as well as exons D7A-1, D7A-2, D7A-3, D8 and D9. Splice variants 4DN1, 4DN2 and 4DN3 (Miro, et al., Biochem. Biophys. Res. Comm., 274: 415-421 (2002), and 4D1, 4D2, 4D3, 4D4 and 4D5 are known (Bolger et al., Biochem. J. pt. 2: 539-548 (1997).

FIG. 4 is a graphic representation showing PDE4D isoform expression in EBV transformed cells (expression of PDE4D3 and PDE4D9 below detection limits).

FIG. 5 is a graphic representation showing expression of PDE4D isoforms in EBV transformed cells from patients with or without the stroke-associated haplotype.

FIG. 6 is a graphic representation showing expression of PDE4D isoforms in EBV cells from controls with or without the stroke-associated haplotype.

FIGS. 7.1 to 7.10 show the amino acid sequences for the isoforms of the PDE4D gene. SEQ ID NO: 2 is D4; SEQ ID NO: 3 is N2; SEQ ID NO: 4 is D5; SEQ ID NO: 5 is N3; SEQ ID NO: 6 is D3; SEQ ID NO: 7 is N1; SEQ ID NO: 8 is D8; SEQ ID NO: 9 is D1; and SEQ ID NO: 10 is D2.

FIGS. 8.1 and 8.2 list all publicly available PDE4D mRNAs and novel cDNA segments identified by deCODE genetics.

FIGS. 9.1 to 9.351 show the genomic sequence of the human PDE4D gene.

FIGS. 10.1 to 10.3 show a graphic representation showing the single marker allelic association within the PDE4D gene. FIG. 10.1 is a schematic showing the gene structures. FIG. 10.2 shows graphic representation of the microsatellite and SNP distribution within the PDE4D gene. FIG. 10.3 shows graphic representation of the single marker allelic association across the PDE4D gene for both microsatellites (filled circles) and SNPs (open circles); negative log p-valve versus the physical location in kilobases.

FIGS. 11.1 to 11.3 graphically depict the haplotype association for carotid and cardiogenic stroke combined. Estimated haplotype frequencies for patients and controls respectively, are indicated within parentheses. FIG. 11.1 is a comparison of groups of haplotypes constructed from SNP45 and AC008818-1, two markers separated by 6 kb. Note that X is a composite allele that denotes jointly all alleles of AC008818-1 except allele 0. Apart from haplotype A0 that is not found in our samples, other haplotypes can be grouped into three groups with distinct risks. Each arrow corresponds to a comparison between two groups and RR is the estimated risk of the group the arrow is pointing at relative to the other group. The difference between 1 and the information (Info) is a measure of the fraction of information that is lost due to uncertainty in phase and missing genotypes. FIG. 11.2 shows intermediate results when the investigation is extended from SNP45 and AC008818-1, which are both in LD block B, to include 25 SNPs in LD block C. H_{C} is the at-risk haplotype, identified in FIG. 13 and L_{C} is a composite haplotype that denotes jointly all haplotypes of the 25 SNPs except H_{C}. Together with AC008818-1 and SNP45, the haplotypes here span 64 kb. Haplotype G0 in A is split into extended haplotypes G0H_{C} and G0L_{C}. G0H_{C} has significantly higher risk than G0L_{C}, and the risk of G0L_{C} is not distinguishable from the wild type GX. FIG. 11.3 shows a refinement of the groupings in A—G0L_{C} is moved from the at-risk group to the wild type group. Also noted is that the extended haplotype AXH_{C} does not exist indicating that blocks B and C are in LD.

FIG. 12 is a schematic representation of the physical map of STRK1 interval showing all genes and mRNAs in region. Markers identified with an asterisk (*) indicate those with significant single marker association.

FIGS. 13.1 to 13.3 show a graphical depiction of the linkage disequilibrium (LD) and haplotypes in the 5′end of PDE4D gene. FIG. 13.1 shows pairwise linkage disequilibrium between SNPs in a 600 kb region in the 5′ end of PDE4D. The markers are plotted equidistant. Two measures of LD are shown: D′ in the upper left triangle and p-values in the lower right triangle. This region can be divided into three blocks of strong LD, each with limited haplotype diversity, block A, block B and block C. The lines indicate the position of the three exons D7-1, D7-2 and D7-3 and the microsatellite marker AC008818-1. FIG. 13.2 show all common haplotypes identified within each of the three blocks. Association results for all the haplotypes are presented in Table 2C. FIG. 13.3 depicts the percentage of chromosomes within each block that match one of the common haplotypes.

### DETAILED DESCRIPTION OF THE INVENTION

The first major stroke locus, STRK1, was mapped to 5q12 using a genome-wide search for susceptibility genes in the common forms of stroke. A broad but rigorous definition of the phenotype was used including patients with ischemic stroke, transient ischemic attack (TIA), and hemorrhagic stroke. The lod score after adding a higher density of markers (one marker every 1 cM) was 4.40 (P=3.9×10⁻⁶) at marker D5S2080. The lod score increased to 4.9 after the hemorrhagic stroke patients were removed, suggesting that the gene at the locus is primarily important for ischemic stroke. The most promising region harboring a stroke susceptibility gene was narrowed down to a segment less than 6 cM (approximately 3.8 Mb), from D5S1474 to D5S398, as defined by a decrease of one in LOD score (will be referred to as the “one-LOD interval” hereafter).

We describe here the positional cloning of a stroke susceptibility gene located in the STRK1 locus. This region was extensively fine-mapped and tested for association to stroke. The strongest association found in the one-LOD interval was within the phosphodiesterase 4D gene (PDE4D), a member of the large superfamily of cyclic nucleotide phosphodiesterases. The strongest signal observed at PDE4D was to the two major subtypes of ischemic stroke, carotid and cardiogenic stroke. Relative expression of PDE4D isoforms correlated with stroke and with the genetic variation within PDE4D which is associated to stroke. Our results suggest that this gene is involved in pathogenesis of stroke through atherosclerosis, the major pathological process underlying stroke.

Our results also indicate that genetic variation in the PDE4D gene is associated with ischemic stroke. The direct involvement of PDE4D is strongly supported by both linkage and haplotype association. Multiple markers and haplotypes within the PDE4D gene show strong association to stroke. The haplotypes can be classified into three distinct groups, wild type, at-risk and protective. We first identified the association using microsatellite markers, and supplementing the microsatellite data with a denser set of SNPs further supported this. The strongest association was to the two ischemic subtypes, carotid and cardiogenic stroke. This gene shows no association to small vessel occlusive disease, the form of stroke thought to be independent of atherosclerosis. Haplotype analyses show that the most significant haplotype extends over an area of 260 kb covering the first exon of the PDE4D gene. The haplotype is significantly associated to carotid and cardiogenic stroke with a relative risk of 2.3 and approximately 47% of carotid/cardiogenic stroke patients carry at least one copy of this haplotype. This same haplotype has a relative risk of 1.8 for stroke in general. This haplotype extends over the 5′exon unique to the PDE4D7 isoform and the presumed promoter region of this isoform suggesting that the functional variation may be involved in transcriptional regulation. This hypothesis is also supported by our PDE4D expression analysis that shows that there is significant correlation between the disease associated haplotype and the level of PDE4D7 message.

The strongest association found for this PDE4D haplotype was to the two major subtypes of ischemic stroke, carotid and cardiogenic stroke suggesting a role for this gene in the vascular biology of atherosclerosis. While there are multiple etiologies for ischemic stroke, atherosclerosis remains the most important one. Atherosclerosis is a chronic progressive disease characterized by accumulation of lipids, fibrous, and cellular elements within the large arteries. These lesions can grow sufficiently large to impede blood flow and, more importantly, their surfaces can rupture leading to local thrombus formation occluding the blood vessel and causing a stroke or myocardial infarction. The major pathological process for the two ischemic subtypes, carotid and cardiogenic stroke is atherosclerosis. First, it is the major cause of stenotic and occlusive lesions of the internal and common carotids that lead to carotid strokes. Second, cardiac thrombi which shed emboli to the brain most commonly occur on the background of coronary artery disease, such as following acute myocardial infarction or ischemic cardiomyopathy, and/or due to atrial fibrillation on the basis of poor compliance of ischemic ventricles (diastolic dysfunction/stiffening). Although atrial fibrillation may occur on the background of other diseases such as valvular disease, hyperthyroidism, and hypertension, in the age group that tends to suffer from stroke, ischemic heart disease remains one of the most important causes. Ischemic stroke resulting from occlusion of small penetrating arteries within the brain (small vessel occlusive disease or lacunar stroke) is generally thought to result from local endothelial proliferation since atherosclerosis only occurs in larger arteries. PDE4D does not show association to small vessel stroke, consistent with it role in atherosclerosis. In summary, atherosclerosis accounts for the majority of all strokes, particularly carotid and cardiogenic stroke, two subphenotypes that show the strongest association to the PDE4D gene.

Representative Target Population

An individual at risk for stroke is an individual who has at least one risk factor, such as previous stroke or TIA, an at-risk haplotype in one or more stroke risk genes, an at-risk haplotype for the PDE4D gene; a polymorphism in a PDE4D gene; disregulation of PDE4D isoform expression; diabetes; hypertension; hypercholesterolemia; elevated lp(a); obesity; a past or current smoker; an elevated inflammatory marker (e.g., a marker such as C-reactive protein (CRP), serum amyloid A, fibrinogen, tissue necrosis factor-alpha, a soluble vascular cell adhesion molecule (sVCAM), a soluble intervascular adhesion molecule (sICAM), E-selectin, matrix metalloprotease type-1, matrix metalloprotease type-2, matrix metalloprotease type-3, and matrix metalloprotease type-9); increased LDL cholesterol and/or decreased HDL cholesterol; and/or at least one previous myocardial infarction, concurrent MI, acute coronary syndrome, stable angina, atherosclerosis, carotid stenosis, peripheral vascular occlusive disease, or requires treatment for restoration of coronary artery blood flow (e.g., angioplasty, stent, coronary artery bypass graft).

An individual who has a protective haplotype is one who is less likely to have a stroke. In another embodiment of the invention, an individual who is at risk for stroke is an individual who has a polymorphism in a PDE4D gene, in which the presence of the polymorphism is indicative of a susceptibility to stroke. An individual who has a protective haplotype and less likely to have a stroke is an individual who has a polymorphism in a PDE4D gene such as the A allele at nucleotide position 142780 relative to SEQ ID NO: 1, in which the presence of the polymorphism is indicative of a protection from stroke. The term “gene,” as used herein, refers to not only the sequence of nucleic acids encoding a polypeptide, but also the promoter regions, transcription enhancement elements, splice donor/acceptor sites, splice enhancer and silencer sequences and other regulators of splicing, and other non-transcribed nucleic acid elements. Representative polymorphisms include those presented in Table 11, below.

In one embodiment of the invention, an individual who is at risk for stroke is an individual who has an at-risk haplotype in PDE4D, as described herein, particularly but not limited to ischemic stroke. Increased risk for the two major subtypes of ischemic stroke, carotid and cardiogenic stroke, can be assessed by screening for at-risk haplotype that comprises SNP5PDM361194, SNP5PDM368135, SNP5PDM370640, SNP5PDM379372 and SNP5PDM408531 at the 5′ UTR of PDE4D7. Results reported herein indicate that PDE4D is involved in pathogenesis of stroke through atherosclerosis. The major pathological process for carotid stroke and cardiogenic stroke is atherosclerosis. Thus, an individual who is at-risk for atherosclerosis, peripheral arterial occlusive disease, or myocardial infarction can also benefit from the teachings of the invention.

Assessment for At-Risk and Protective Haplotypes

A “haplotype,” as described herein, refers to a combination of genetic markers (“alleles”), such as those set forth in Tables 1, 2C, 4A and 4B. In a certain embodiment, the haplotype can comprise one or more alleles, two or more alleles, three or more alleles, four or more alleles, or five or more alleles. The genetic markers are particular “alleles” at “polymorphic sites” associated with PDE4D. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference PDE4D sequence is described herein by SEQ ID NO: 1. The term, “variant PDE4D”, as used herein, refers to a sequence that differs from SEQ ID NO: 1, but is otherwise substantially similar. The genetic markers that make up the haplotypes described herein are PDE4D variants.

Additional variants can include changes that affect a polypeptide, e.g., the PDE4D polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a PDE4D nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with stroke or a susceptibility to stroke can be a synonymous change in one or more nucleotides (i.e., a change that does not result in a change in the amino acid sequence). Such a polymorphism can, for example, alter splice sites, affect, the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein, e.g., having markers such as those shown in Table 3, Table 4A and 4B, are found more frequently in individuals with stroke than in individuals without stroke. Therefore, these haplotypes have predictive value for detecting stroke or a susceptibility to stroke in an individual. The haplotypes described herein are a combination of various genetic markers, e.g., SNPs and microsatellites. Therefore, detecting haplotypes can be accomplished by methods known in the art for detecting sequences at polymorphic sites, such as the methods described above.

In certain methods described herein, an individual who is at risk for stroke is an individual in whom an at-risk haplotype is identified. In one embodiment, the at-risk haplotype is one that confers a significant risk of stroke. In one embodiment, significance associated with a haplotype is measured by an odds ratio. In a further embodiment, the significance is measured by a percentage. In one embodiment, a significant risk is measured as an odds ratio of at least about 1.2, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

An at-risk haplotype in, or comprising portions of, the PDE4D gene, is one where the haplotype is more frequently present in an individual at risk for stroke (affected), compared to the frequency of its presence in a healthy individual (control), and wherein the presence of the haplotype is indicative of stroke or susceptibility to stroke. A protective haplotype in or comprising portions of the PDE4D gene is one where the haplotype is more frequently present in an individual where the haplotype is protective against being affected by stroke compared to the frequency of its presence in an individual with stroke. The presence of the haplotype is indicative of a protection from stroke or protection from susceptibility to stroke as described above.

Standard techniques for genotyping for the presence of SNPs and/or microsatellite markers can be used, such as fluorescent-based techniques (Chen, et al., Genome Res. 9, 492 (1999)), PCR, LCR, Nested PCR and other techniques for nucleic acid amplification. In one embodiment, the method comprises assessing in an individual the presence or frequency of SNPs and/or microsatellites in, comprising portions of, the PDE4Dgene, wherein an excess or higher frequency of the SNPs and/or microsatellites compared to a healthy control individual is indicative that the individual has stroke, or is susceptible to stroke. See, for example, Table 1, Table 2C, Table 2D, Table 3, Table 4A and 4B (below) for SNPs and markers that can form haplotypes that can be used as screening tools. These markers and SNPs can be identified in at-risk haploptypes. For example, an at-risk haplotype can include microsatellite markers and/or SNPs such as those set forth in Table 2C, Table 4B and 4B. The presence of the haplotype is indicative of stroke, or a susceptibility to stroke, and therefore is indicative of an individual who falls within a target population for the treatment methods described herein.

Haplotype analysis first involves defining a candidate susceptibility locus using LOD scores. The defined regions are then ultra-fine mapped with microsatellite markers with an average spacing between markers of less than 100 kb. All usable microsatellite markers that found in public databases and mapped within that region can be used. In addition, microsatellite markers identified within the deCODE genetics sequence assembly of the human genome can be used. The frequencies of haplotypes in the patient and the control groups using an expectation-maximization algorithm can be estimated (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype, which can include the markers described herein, is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistics is used to evaluate the statistic significance.

To look for at-risk-haplotypes in the 1-lod drop or protective haplotypes, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values.

In one embodiment, the at-risk haplotype is characterized by the presence of the polymorphism(s) represented by one or a combination of single nucleotide polymorphisms at nucleic acid positions 1425923, 1415979, 1414804, 1371388, 1307403 and 1257206, relative to SEQ ID NO: 1. In another embodiment, a diagnostic method for susceptibility to stroke can comprise determining the presence of at-risk haplotype represented by one or a combination of single nucleotide polymorphisms and microsatellite markers at nucleic acid positions 263539, 252772, 189780, 175259, 171240, 136550 and 120628, relative to SEQ ID NO: 1. In another embodiment, the at-risk haplotype is characterized by the following SNPs: SNP5PDM361194, SNP5PDM368135, SNP5PDM370640, SNP5PDM379372, and SNP5PDM408531. In one embodiment, the protective haplotype comprises the A allele of SNP45 at position 142780 relative to SEQ ID NO: 1. This haplotype is particularly useful for assessing susceptibility to the two major subtypes of ischemic stroke, carotid and cardiogenic stroke. In another embodiment, an at-risk haplotype, particularly for carotid and cardiogenic stroke, is characterized by use of microsatellite marker AC008818-1 to define the presence of an at-risk allele.

Nucleic Acid Therapeutic Agents

In another embodiment, a nucleic acid of the invention; a nucleic acid complementary to a nucleic acid of the invention; or a portion of such a nucleic acid (e.g., an oligonucleotide as described below); or a nucleic acid encoding a PDE4D polypeptide, can be used in “antisense” therapy, in which a nucleic acid (e.g., an oligonucleotide) which specifically hybridizes to the mRNA and/or genomic DNA of a nucleic acid is administered or generated in situ. The antisense nucleic acid that specifically hybridizes to the mRNA and/or DNA inhibits expression of the polypeptide encoded by that mRNA and/or DNA, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes a PDE4D polypeptide. Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Additionally, general approaches to constructing oligomers useful in antisense therapy are also described, for example, by Van der Krol et al. (Biotechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the polypeptide. The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication No. WO 88/09810) or the blood-brain barrier (see, e.g., PCT International Publication No. WO 89/10134), or hybridization-triggered cleavage agents (see, e.g., Krol et al., BioTechniques 6:958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express a PDE4D polypeptide in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a another embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

In another embodiment of the invention, small double-stranded interfering RNA (RNA interference (RNAi)) can be used. RNAi is a post-transcription process, in which double-stranded RNA is introduced, and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al., Nature Medicine 8(7):681-686 (2002); the entire teachings of these references are incorporated herein by reference.

Endogenous expression of a gene product can also be reduced by inactivating or “knocking out” the gene or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of an gene in the cell, as described above. In another embodiment, targeted homologous recombination can be used to insert a DNA construct comprising a nucleic acid that encodes a polypeptide variant that differs from that present in the cell.

Alternatively, endogenous expression of a gene product can be reduced by targeting deoxyribonucleotide sequences complementary to the regulatory region (i.e., the promoter and/or enhancers) to form triple helical structures that prevent transcription of the gene in target cells in the body. (See generally, Helene, C., Anticancer Drug Des., 6(6):569-84 (1991); Helene, C. et al., Ann. N.Y. Acad. Sci. 660:27-36 (1992); and Maher, L. J., Bioassays 14(12):807-15 (1992)). Likewise, the antisense constructs described herein, by antagonizing the normal biological activity of the gene product, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a nucleic acid RNA or nucleic acid sequence) can be used to investigate the role of one or more members of the PDE4D pathway in the development of disease-related conditions. Such techniques can be utilized in cell culture, but can also be used in the creation of transgenic animals.

The therapeutic agents as described herein can be delivered in a composition, as described above, or alone. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. In addition, a combination of any of the above methods of treatment (e.g., administration of non-altered polypeptide in conjunction with antisense therapy targeting altered mRNA; administration of a first splicing variant in conjunction with antisense therapy targeting a second splicing variant) can also be used.

The invention additionally pertains to use of such therapeutic agents, as described herein, for the manufacture of a medicament for the treatment of stroke, TIA, MI, and/or atherosclerosis, e.g., using the methods described herein.

Monitoring Progress of Treatment

The current invention also pertains to methods of monitoring the effectiveness of treatment on the regulation of expression (e.g., relative or absolute expression) of one or more PDE4D isoforms at the RNA or protein level or its enzymatic activity. PDE4D message or protein or enzymatic activity can be measured in a sample of peripheral blood or cells derived therefrom. An assessment of the levels of expression or activity can be made before and during treatment with PDE4D therapeutic agents.

For example, in one embodiment of the invention, an individual who is a member of the target population can be assessed for response to treatment with a PDE4D inhibitor, by examining cAMP levels or PDE4D enzymatic activity or absolute and/or relative levels of PDE4D protein or mRNA isoforms in peripheral blood in general or specific cell subfractions or combination of cell subfractions. In addition, variation such as haplotypes or mutations within or near (within 100 to 200 kb) of the PDE4D gene may be used to identify individuals who are at higher risk for stroke or TIA to increase the power and efficiency of clinical trials for pharmaceutical agents to prevent or treat first or subsequent stroke. The haplotypes and other variations may be used to exclude or fractionate patients in a clinical trial who are likely to have non-cAMP or non-PDE4D pathway involvement in their stroke risk in order to enrich patients who have other pathways involved and boost the power and sensitivity of the clinical trial. Such variation may be used as a pharmacogenomic test to guide selection of pharmaceutical agents for individuals.

Nucleic Acids of the Invention

Nucleic Acids, Portions and Variants

All nucleotide positions are relative to SEQ ID NO: 1. The nucleic acids, polypeptides and antibodies described herein can be used in methods of diagnosis of susceptibility to stroke, as well as in kits useful for diagnosis of a susceptibility to stroke. In addition, the invention pertains to isolated nucleic acid molecules comprising a human PDE4D nucleic acid. The term, “PDE4D nucleic acid,” as used herein, refers to an isolated nucleic acid molecule encoding PDE4D polypeptide. The PDE4D nucleic acid molecules of the present invention can be RNA, for example, mRNA, or DNA, such as cDNA and genomic DNA. DNA molecules can be double-stranded or single-stranded; single stranded RNA or DNA can be either the coding, or sense strand or the non-coding, or antisense strand. The nucleic acid molecule can include all or a portion of the coding sequence of the gene or nucleic acid and can further comprise additional non-coding sequences such as introns and non-coding 3′ and 5′ sequences (including regulatory sequences, for example, as well as promoters, transcription enhancement elements, splice donor/acceptor sites, etc.). For example, a PDE4D nucleic acid can comprise the nucleic acid of SEQ ID NO: 1 which may optionally comprise at least one polymorphism as shown in Tables 11 and 12, the complement thereof, or to a portion or fragment of such an isolated nucleic acid molecule (e.g., cDNA or the nucleic acid) that encodes PDE4D polypeptide.

Additionally, the nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to variant nucleic acid molecules which are not necessarily found in nature but which encode a PDE4D polypeptide (e.g., a polypeptide having the amino acid sequence of SEQ ID NO: 2, 3, 4, 5, 6, 7, 8, 9, 10, 12 or 14), or another splicing variant of PDE4D polypeptide or polymorphic variant thereof. Thus, for example, DNA molecules which comprise a sequence that is different from the naturally-occurring nucleotide sequence but which, due to the degeneracy of the genetic code, encode a PDE4D polypeptide of the present invention are also the subject of this invention. The invention also encompasses nucleotide sequences encoding portions (fragments), or encoding variant polypeptides such as analogues or derivatives of the PDE4D polypeptide. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of the PDE4D polypeptide. In one embodiment, the nucleotide sequences are fragments that comprise one or more polymorphic microsatellite markers. In another embodiment, the nucleotide sequences are fragments that comprise one or more single nucleotide polymorphisms in the PDE4D gene.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence comprising a nucleotide sequence selected from SEQ ID NO: 1 which may optionally comprise at least one polymorphism as shown in Tables 11 and 12 or the complement thereof. In another embodiment, the invention includes variants described herein which hybridize under high stringency hybridization conditions (e.g., for selective hybridization) to a nucleotide sequence encoding an amino acid sequence selected from SEQ ID NO: 2, 3, 4, 5, 6, 7, 8, 9, 10, 12 or 14 or polymorphic variant thereof. In another embodiment, the protein product of the variant that hybridizes under high stringency conditions has an activity of PDE4D.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of ˜17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. One, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

In another embodiment, the percent identity between two amino acid sequences can be accomplished using the GAP program in the GCG software package (Accelrys, Cambridge, UK) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO: 1 which may optionally comprise at least one polymorphism as shown in Tables 11 and 12 and the complement thereof, and also provides isolated nucleic acid molecules that contain a fragment or portion that hybridizes under highly stringent conditions to a nucleotide sequence encoding an amino acid sequence selected from SEQ ID NO: 2, 3, 4, 5, 6, 7, 8, 9, 10, 12 or 14, or polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarities. Such probes and primers include polypeptide nucleic acids, as described in Nielsen et al., Science, 254, 1497-1500 (1991).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence of SEQ ID NO: 1 or the complement of SEQ ID NO: 1, or a nucleic acid sequence encoding an amino acid sequence of SEQ ID NO: 2, 3, 4, 5, 6, 7, 8, 9, 10, 12 or 14, or polymorphic variant thereof. In certain embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on one or more of the sequences provided in SEQ ID NO: 1 which may optionally comprise at least one polymorphism shown in Tables 11 and 12, and/or the complement thereof, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics, 4:560 (1989), Landegren et al., Science, 241:1077 (1988), transcription amplification (Kwoh-et al., Proc. Natl. Acad. Sci. USA, 86:1173 (1989)), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled (e.g., with radiolabel or other reporter molecule) and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide and the DNA encoding the polypeptide can be isolated, sequenced and further characterized.

Antisense nucleic acid molecules of the invention can be designed using the nucleotide sequences of SEQ ID NO: 1 and/or the complement of SEQ ID NO: 1, and/or a portion of SEQ ID NO: 1 or the complement of SEQ ID NO: 1 and/or a sequence encoding the amino acid sequences or SEQ ID NO: 2, 3, 4, 5, 6, 7, 8, 9, 10, 12 and/or 14, or encoding a portion of SEQ ID NO: 2, 3, 4, 5, 6, 7, 8, 9, 10, 12 and/or 14, (wherein any one of these may optionally comprise at least one polymorphism as shown in Tables 11 and 12) and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify genetic disorders (e.g., a predisposition for or susceptibility to stroke), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors

Another aspect of the invention pertains to nucleic acid constructs containing a nucleic acid molecule selected from the group consisting of SEQ ID NO: 1 which may optionally comprise at least one polymorphism shown in Tables 11 and 12 and the complement thereof (or a portion thereof). Yet another aspect of the invention pertains to nucleic acid constructs containing a nucleic acid molecule encoding the amino acid sequence of SEQ ID NO: 2, 3, 4, 5, 6, 7, 8, 9, 10, 12 or 14 or polymorphic variant thereof. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably or operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al., (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous PDE4D gene, or an exogenous nucleic acid encoding PDE4D polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology, 2:823-829 and in PCT Publication Nos. WO 90/11354, WO 91/01140, WO 92/0968, and WO 93/04169. Clones of the non-human transgenic animals described herein can also be produced according to the methods described in Wilmut et al. (1997) Nature, 385:810-813 and PCT Publication Nos. WO 97/07668 and WO 97/07669.

Polypeptides of the Invention

The present invention also pertains to isolated polypeptides encoded by PDE4D (“PDE4D polypeptides”) and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence selected from the group consisting of SEQ ID NO: 1 which may optionally comprise at least one polymorphism shown in Tables 11 and 12 and complements and portions thereof, e.g., SEQ ID NO: 2, 3, 4, 5, 6, 7, 8, 9, 10, 12 or 14, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide sequence selected from the group consisting of SEQ ID NO: 1 which may optionally comprise at least one polymorphism shown in Tables 11 and 12 and complements and portions thereof, or having substantial homology to a polypeptide encoded by a nucleic acid molecule comprising a nucleotide sequence selected from the group consisting of nucleotide sequences encoding SEQ ID NO: 2, 3, 4, 5, 6, 7, 8, 9, 10, 12 or 14, or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in others greater than about 90% or more homologous or identical. A substantially homologous amino acid sequence, according to the present invention, will be encoded by a nucleic acid molecule hybridizing to SEQ ID NO: 1 which may optionally comprise at least one polymorphism shown in Tables 11 and 12, or portion thereof, under stringent conditions as more particularly described above, or will be encoded by a nucleic acid molecule hybridizing to a nucleic acid sequence encoding SEQ ID NO: 2, 3, 4, 5, 6, 7, 8, 9, 10, 12 or 14, portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol., 224:899-904 (1992); de Vos et al., Science, 255:306-312 (1992)).

The invention also includes polypeptide fragments of the polypeptides of the invention. Fragments can be derived from a polypeptide encoded by a nucleic acid molecule comprising SEQ ID NO: 1 which may optionally comprise at least one polymorphism shown in Tables 11 and 12 or a portion thereof and the complements thereof (e.g., SEQ ID NO: 2, 3, 4, 5, 6, 7, 8, 9, 10, 12 or 14, or other splicing variants). However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., receptor or ligand, such as, for example, in an interaction trap assay, and to screen for peptide or small molecule antagonists or agonists of the binding interaction.

Antibodies of the Invention

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The invention provides antibodies to the polypeptides and polypeptide fragments of the invention, e.g., having an amino acid sequence encoded by SEQ ID NO: 2, 3, 4, 5, 6, 7, 8, 9, 10, 12 or 14, or a portion thereof, or having an amino acid sequence encoded by a nucleic acid molecule comprising all or a portion of SEQ ID NO: 1 which may optionally comprise at least one polymorphism shown in Tables 11 and 12 (e.g., SEQ ID NO: 2, 3, 4, 5, 6, 7, 8, 9, 10, 12 or 14, or another splicing variant or portion thereof). The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Coupling the antibody to a detectable substance can facilitate detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Diagnostic Assays

The nucleic acids, probes, primers, polypeptides and antibodies described herein can be used in methods of diagnosis of stroke or diagnosis of a susceptibility to stroke or to a disease or condition associated with an stroke gene, such as PDE4D, as well as in kits useful for diagnosis of stroke or a susceptibility to stroke or to a disease or condition associated with PDE4D. In one embodiment, the kit useful for diagnosis of stroke or susceptibility to stroke, or to a disease or condition associated with PDE4D comprises primers as described herein, wherein the primers contain one or more of the SNPs identified herein. In parallel, definition of stroke risk associated with PDE4D/cAMP pathway is useful and novel to define subgroups of individuals who would be best treated by pharmaceutical agents acting on PDE4D and/cAMP pathways (and vice versa).

In one embodiment of the invention, diagnosis of stroke or susceptibility to stroke (or diagnosis of or susceptibility to a disease or condition associated with PDE4D) is made by detecting a polymorphism in a PDE4D nucleic acid as described herein. The polymorphism can be an alteration in a PDE4D nucleic acid, such as the insertion or deletion of a single nucleotide, or of more than one nucleotide, resulting in a frame shift alteration; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene or nucleic acid; duplication of all or a part of the gene or nucleic acid; transposition of all or a part of the gene or nucleic acid; or rearrangement of all or a part of the gene or nucleic acid. More than one such alteration may be present in a single gene or nucleic acid. Such sequence changes cause an alteration in the polypeptide encoded by a PDE4D nucleic acid. For example, if the alteration is a frame shift alteration, the frame shift can result in a change in the encoded amino acids, and/or can result in the generation of a premature stop codon, causing generation of a truncated polypeptide. Alternatively, a polymorphism associated with a disease or condition associated with a PDE4D nucleic acid or a susceptibility to a disease or condition associated with a PDE4D nucleic acid can be a synonymous alteration in one or more nucleotides (i.e., an alteration that does not result in a change in the polypeptide encoded by a PDE4D nucleic acid). For diagnostic applications, there may be polymorphisms informative for prediction of disease risk that are in linkage disequilibrium with the functional polymorphism. Such a polymorphism may alter splicing sites, affect the stability or transport of mRNA, or otherwise affect the transcription or translation of the nucleic acid. A PDE4D nucleic acid that has any of the alteration described above is referred to herein as an “altered nucleic acid.”

In a first method of diagnosing stroke or a susceptibility to stroke, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, a susceptibility to a disease or condition associated with a PDE4D nucleic acid (the “test individual”). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in a stroke nucleic acid is present, and/or to determine which splicing variant(s) encoded by the PDE4D is present. The presence of the polymorphism or splicing variant(s) can be indicated by hybridization of the nucleic acid in the genomic DNA, RNA, or cDNA to a nucleic acid probe. A “nucleic acid probe,” as used herein, can be a DNA probe or an RNA probe; the nucleic acid probe can contain at least one polymorphism in a PDE4D nucleic acid or contains a nucleic acid encoding a particular splicing variant of a PDE4D nucleic acid. The probe can be any of the nucleic acid molecules described above (e.g., the nucleic acid, a fragment, a vector comprising the nucleic acid, a probe or primer, etc.).

To diagnose a susceptibility to stroke, a hybridization sample is formed by contacting the test sample containing PDE4D, with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO: 1 which may optionally comprise at least one polymorphism shown in Tables 11 and 12, or the complement thereof, or a portion thereof; or can be a nucleic acid encoding a portion of SEQ ID NO: 2, 3, 4, 5, 6, 7, 8, 9, 10, 12 or 14. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to PDE4D. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and PDE4D in the test sample, then PDE4D has the polymorphism, or is the splicing variant, that is present in the nucleic acid probe. More than one nucleic acid probe can also be used concurrently in this method. In one embodiment, specific hybridization of at least one of the nucleic acid probes is indicative of a polymorphism in PDE4D, or of the presence of a particular splicing variant encoding PDE4D and is therefore diagnostic for a susceptibility to stroke.

In Northern analysis (see Current Protocols in Molecular Biology, Ausubel, F. et al., eds., John Wiley & Sons, supra) the hybridization methods described above are used to identify the presence of a polymorphism or a particular splicing variant, associated with a susceptibility to stroke. For Northern analysis, a test sample of RNA is obtained from the individual by appropriate means. Specific hybridization of a nucleic acid probe, as described above, to RNA from the individual is indicative of a polymorphism in PDE4D, or of the presence of a particular splicing variant encoded by PDE4D, and is therefore diagnostic for a susceptibility to stroke.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a gene having a polymorphism associated with a susceptibility to stroke. Hybridization of the PNA probe to PDE4D is diagnostic for a susceptibility to stroke.

In another method of the invention, mutation analysis by restriction digestion can be used to detect a mutant gene, or genes containing a polymorphism(s), if the mutation or polymorphism in the gene results in the creation or elimination of a restriction site. If a restriction site is not naturally created, one can be created by PCR that depends on the polymorphism and allows genotyping. A test sample containing genomic DNA is obtained from the individual. Nucleic acid amplification methods, including but not limited to Polymerase chain reaction (PCR), Transcription Mediated Amplifications (TMA), and Ligase Mediate Amplification (LMA), can be used to amplify PDE4D. The digestion pattern of the relevant DNA fragment indicates the presence or absence of the mutation or polymorphism in PDE4D, and therefore indicates the presence or absence of this susceptibility to stroke. RFLP analysis is conducted as described (see Current Protocols in Molecular Biology, supra). Amplification techniques based upon detection of sequence of interest using reverse dot blot technology (linear array or strips) can be used and are described, for example, in U.S. Pat. No. 5,468,613.

Sequence analysis can also be used to detect specific polymorphisms in PDE4D. A test sample of DNA or RNA is obtained from the test individual. PCR or other appropriate methods can be used to amplify the gene, and/or its flanking sequences, if desired. The sequence of PDE4D, or a fragment of the gene, or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA (e.g., SEQ ID NO: 1 which may optionally comprise at least one polymorphism shown in Tables 11 and 12, or a nucleic acid sequence encoding SEQ ID NO: 2, 3, 4, 5, 6, 7, 8, 9, 10, 12 or 14, or a fragment thereof) or mRNA, as appropriate. In one embodiment, the presence of at least one of the polymorphisms in PDE4D indicates that the individual has a susceptibility to stroke.

Allele-specific oligonucleotides can also be used to detect the presence of a polymorphism in PDE4D, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to PDE4D, and that contains a polymorphism associated with a susceptibility to stroke. An allele-specific oligonucleotide probe that is specific for particular polymorphisms in PDE4D can be prepared, using standard methods (see Current Protocols in Molecular Biology, supra). To identify polymorphisms in the gene that are associated with a susceptibility to stroke, a test sample of DNA is obtained from the individual. PCR can be used to amplify all or a fragment of PDE4D, and its flanking sequences. The DNA containing the amplified PDE4D (or fragment of the gene) is dot-blotted, using standard methods (see Current Protocols in Molecular Biology, supra), and the blot is contacted with the oligonucleotide probe. The presence of specific hybridization of the probe to the amplified PDE4D is then detected. Specific hybridization of an allele-specific oligonucleotide probe to DNA from the individual is indicative of a polymorphism in PDE4D, and is therefore indicative of a susceptibility to stroke.

The invention further provides allele-specific oligonucleotides that hybridize to the reference or variant allele of a nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer that hybridizes at a distal site. Amplification proceeds from the two primers, resulting in a detectable product that indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another embodiment, arrays of oligonucleotide probes that are complementary to target nucleic acid sequence segments from an individual, can be used to identify polymorphisms in PDE4D. For example, in one embodiment, an oligonucleotide linear array can be used. Oligonucleotide arrays typically comprise a plurality of different oligonucleotide probes that are coupled to a surface of a substrate in different known locations. These oligonucleotide arrays, also described as “Genechips.™.,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein. In another embodiment, linear arrays or microarrays can be utilized.

Once an oligonucleotide array is prepared, a nucleic acid of interest is hybridized with the array and scanned for polymorphisms. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in PDE4D or splicing variants encoding by PDE4D. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al. (19891) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers, R. M. et al. (1985) Science 230:1242); use of polypeptides which recognize nucleotide mismatches, such as E. coli mutS protein, for example.

In one embodiment of the invention, diagnosis of a disease or condition associated with PDE4D (e.g., stroke) or a susceptibility to a disease or condition associated with PDE4D (e.g., stroke) can also be made by expression analysis by quantitative PCR (kinetic thermal cycling). This technique utilizing TaqMan® or Lightcycler® can be used to allow the identification of polymorphisms and whether a patient is homozygous or heterozygous. The technique can assess the presence of an alteration in the expression or composition of the polypeptide encoded by a PDE4D nucleic acid or splicing variants encoded by a PDE4D nucleic acid. Further, the expression of the variants can be quantified as physically or functionally different.

In another embodiment of the invention, diagnosis of a susceptibility to stroke can also be made by examining expression and/or composition of an PDE4D polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by PDE4D, or for the presence of a particular variant (e.g., an isoform) encoded by PDE4D. An alteration in expression of a polypeptide encoded by PDE4D can be, for example, an alteration in the quantitative polypeptide expression (i.e., the amount of polypeptide produced); an alteration in the composition of a polypeptide encoded by PDE4D is an alteration in the qualitative polypeptide expression (e.g., expression of a mutant PDE4D polypeptide or of a different splicing variant or isoform). In one embodiment, detecting a particular splicing variant encoded by that PDE4D, or a particular pattern of splicing variants makes diagnosis of the disease or condition associated with PDE4D or a susceptibility to a disease or condition associated with PDE4D.

Both such alterations (quantitative and qualitative) can also be present. An “alteration” in the polypeptide expression or composition, as used herein, refers to an alteration in expression or composition in a test sample, as compared with the expression or composition of polypeptide by PDE4D in a control sample. A control sample is a sample that corresponds to the test sample (e.g., is from the same type of cells), and is from an individual who is not affected by stroke. An alteration in the expression or composition of the polypeptide in the test sample, as compared with the control sample, is indicative of a susceptibility to stroke. Similarly, the presence of one or more different splicing variants or isoforms in the test sample, or the presence of significantly different amounts of different splicing variants in the test sample, as compared with the control sample, is indicative of a susceptibility to stroke. Various means of examining expression or composition of the polypeptide encoded by PDE4D can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by a mutant PDE4D, or an antibody that specifically binds to a polypeptide encoded by a non-mutant gene, or an antibody that specifically binds to a particular splicing variant encoded by PDE4D, can be used to identify the presence in a test sample of a particular splicing variant or isoform, or of a polypeptide encoded by a polymorphic or mutant PDE4D, or the absence in a test sample of a particular splicing variant or isoform, or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to stroke, as is the presence (or absence) of particular splicing variants encoded by the PDE4D gene.

In one embodiment of this method, the level or amount of polypeptide encoded by PDE4D in a test sample is compared with the level or amount of the polypeptide encoded by PDE4D in a control sample. A level or amount of the polypeptide in the test sample that is higher or lower than the level or amount of the polypeptide in the control sample, such that the difference is statistically significant, is indicative of an alteration in the expression of the polypeptide encoded by PDE4D, and is diagnostic for a susceptibility to stroke. Alternatively, the composition of the polypeptide encoded by PDE4D in a test sample is compared with the composition of the polypeptide encoded by PDE4D in a control sample (e.g., the presence of different splicing variants). A difference in the composition of the polypeptide in the test sample, as compared with the composition of the polypeptide in the control sample, is diagnostic for a susceptibility to stroke. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to stroke.

In another embodiment, assessment of the splicing variant or isoform(s) of a polypeptide encoded by a polymorphic or mutant PDE4D, can be performed. The assessment can be performed directly (e.g., by examining the polypeptide itself), or indirectly (e.g., by examining the mRNA encoding the polypeptide, such as through mRNA profiling). For example, probes or primers as described herein can be used to determine which splicing variants or isoforms are encoded by PDE4D mRNA, using standard methods.

The presence in a test sample of a particular splicing variant(s) or isoform(s) associated with stroke or risk of stroke, or the absence in a test sample of a particular splicing variant(s) or isoform(s) not associated with stroke or risk of stroke, is diagnostic for a disease or condition associated with a PDE4D gene or a susceptibility to a disease condition associated with a PDE4D gene. Similarly, the absence in a test sample of a particular splicing variant(s) or isoform(s) associated with stroke or risk of stroke, or the presence in a test sample of a particular splicing variant(s) or isoform(s) not associated with stroke or risk of stroke, is diagnostic for the absence of disease or condition associated with a PDE4D gene or a susceptibility to a disease or condition associated with a PDE4D gene.

In another embodiment, differential expression of isoforms PDE4D7, PDE4D9 and combinations thereof can be assessed and compared to control individuals. Decreased expression of these isoforms is indicative of susceptibility to stroke, particularly carotid stroke and/or cardiogenic stroke.

The invention further pertains to a method for the diagnosis and identification of susceptibility to stroke in an individual, by identifying an at-risk haplotype in PDE4D. In one embodiment, the at-risk haplotype is a haplotype for which the presence of the haplotype increases the risk of stroke significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of at least about 1.2, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

The invention also pertains to methods of diagnosing stroke or a susceptibility to stroke in an individual, comprising screening for an at-risk haplotype in the PDE4D nucleic acid that is more frequently present in an individual susceptible to stroke (affected), compared to the frequency of its presence in a healthy individual (control), wherein the presence of the haplotype is indicative of stroke or susceptibility to stroke. Standard techniques for genotyping for the presence of SNPs and/or microsatellite markers that are associated with stroke can be used, such as fluorescent-based techniques (Chen, et al., Genome Res. 9, 492 (1999), PCR, LCR, Nested PCR and other techniques for nucleic acid amplification. In one embodiment, the method comprises assessing in an individual the presence or frequency of SNPs and/or microsatellites in the PDE4D nucleic acid that are associated with stroke, wherein an excess or higher frequency of the SNPs and/or microsatellites compared to a healthy control individual is indicative that the individual has stroke or is susceptible to stroke.

See Table 2C, Table 3, Table 4A, and 4B for SNPs and markers that comprise haplotypes that can be used as screening tools. See also, Table 5, Table 6, Table 11 and Table 12 that set forth previously known SNP and novel microsatellite markers and their counterpart sequence ID reference numbers. SNPs and markers from these lists represent at-risk haplotypes and can be used to design diagnostic tests for determining a susceptibility to stroke.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) PDE4D polypeptide, means for amplification of nucleic acids comprising PDE4D, or means for analyzing the nucleic acid sequence of PDE4D or for analyzing the amino acid sequence of an PDE4D polypeptide, etc. In one embodiment, a kit for diagnosing susceptibility to stroke can comprise primers for nucleic acid amplification of a region in the PDE4D gene comprising an at-risk haplotype that is more frequently present in an individual susceptible to stroke. The primers can be designed using portions of the nucleic acids flanking SNPs that are indicative of stroke. In a particularly preferred embodiment, the primers are designed to amplify regions of the PDE4D gene associated with an at-risk haplotype for stroke, shown in Tables 8A and 8B. In another embodiment of the invention, a kit for diagnosing susceptibility to stroke can further comprise probes designed to hybridize to regions of the PDE4D gene associated with an at-risk haplotype for stroke, shown in Table 5 and table 6 and/or generated from SEQ ID Nos: 85-102.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of SEQ ID NO: 1 which may optionally comprise at least one polymorphism shown in Tables 11 and 12, or the complement thereof, or a nucleic acid encoding an amino acid having the sequence of SEQ ID NO: 2, 3, 4, 5, 6, 7, 8, 9, 10, 12 or 14, or a fragment or variant of such nucleic acids), under stringent conditions as described above, and then assessing the sample for the presence (or absence) of hybridization. In another embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a PDE4D nucleic acid), and the contacted sample is assessed for the presence or absence of hybridization. In another embodiment, the nucleic acid containing a contiguous nucleotide sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes) that alter (e.g., increase or decrease) the activity of the polypeptides described herein, or which otherwise interact with the polypeptides herein. For example, such agents can be agents which bind to polypeptides described herein (e.g., PDE4D binding agents); which have a stimulatory or inhibitory effect on, for example, activity of polypeptides of the invention; or which change (e.g., enhance or inhibit) the ability of the polypeptides of the invention to interact with PDE4D binding agents (e.g., receptors or other binding agents); or which alter posttranslational processing of the PDE4D polypeptide (e.g., agents that alter proteolytic processing to direct the polypeptide from where it is normally synthesized to another location in the cell, such as the cell surface); agents that alter proteolytic processing such that more polypeptide is released from the cell, etc.

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des., 12:145).

In one embodiment, to identify agents which alter the activity of a PDE4D polypeptide, a cell, cell lysate, or solution containing or expressing a PDE4D polypeptide (e.g., SEQ ID NO: 2, 3, 4, 5, 6, 7, 8, 9, 10, 12 or 14, or another splicing variant encoded by PDE4D), or a fragment or derivative thereof (as described above), can be contacted with an agent to be tested; alternatively, the polypeptide can be contacted directly with the agent to be tested. The level (amount) of PDE4D activity is assessed (e.g., the level (amount) of PDE4D activity is measured, either directly or indirectly), and is compared with the level of activity in a control (i.e., the level of activity of the PDE4D polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of PDE4D polypeptide. An increase in the level of PDE4D activity relative to level of the control, indicates that the agent is an agent that enhances (is an agonist of) PDE4D activity. Similarly, a decrease in the level of PDE4D activity relative to level of the control, indicates that the agent is an agent that inhibits (is an antagonist of) PDE4D activity. In another embodiment, the level of activity of a PDE4D polypeptide or derivative or fragment thereof in the presence of the agent to be tested, is compared with a control level that has previously been established. A level of the activity in the presence of the agent that differs from the control level by an amount that is statistically significant indicates that the agent alters PDE4D activity.

The present invention also relates to an assay for identifying agents which alter the expression of the PDE4D gene (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding PDE4D polypeptide (e.g., PDE4D gene) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of PDE4D expression (e.g., the level and/or pattern of mRNA or of protein expressed, such as the level and/or pattern of different splicing variants) is assessed, and is compared with the level and/or pattern of expression in a control (i.e., the level and/or pattern of the PDE4D expression in the absence of the agent to be tested). If the level and/or pattern in the presence of the agent differ, by an amount or in a manner that is statistically significant, from the level and/or pattern in the absence of the agent, then the agent is an agent that alters the expression of PDE4D. Enhancement of PDE4D expression indicates that the agent is an agonist of PDE4D activity. Similarly, inhibition of PDE4D expression indicates that the agent is an antagonist of PDE4D activity. In another embodiment, the level and/or pattern of PDE4D polypeptide(s) (e.g., different splicing variants) in the presence of the agent to be tested, is compared with a control level and/or pattern that have previously been established. A level and/or pattern in the presence of the agent that differs from the control level and/or pattern by an amount or in a manner that is statistically significant indicates that the agent alters PDE4D expression. In one embodiment, agents that can alter expression levels of isoforms PDE4D7 and/or PDE4D9 can be assessed, preferably to complement the expression levels to approximate the ratios of a healthy individual.

In another embodiment of the invention, agents which alter the expression of the PDE4D gene or which otherwise interact with the nucleic acids described herein, can be identified using a cell, cell lysate, or solution containing a nucleic acid encoding the promoter region of the PDE4D gene operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of PDE4D, as indicated by its ability to alter expression of a gene that is operably linked to the PDE4D gene promoter. Enhancement of the expression of the reporter indicates that the agent is an agonist of PDE4D activity. Similarly, inhibition of the expression of the reporter indicates that the agent is an antagonist of PDE4D activity. In another embodiment, the level of expression of the reporter in the presence of the agent to be tested, is compared with a control level that has previously been established. A level in the presence of the agent that differs from the control level by an amount or in a manner that is statistically significant indicates that the agent alters PDE4D expression.

Agents which alter the amounts of different splicing variants encoded by PDE4D (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above. In other embodiments of the invention, assays can be used to assess the impact of a test agent on the activity of a polypeptide in relation to a PDE4D binding agent. For example, a cell that expresses a compound that interacts with PDE4D (herein referred to as a “PDE4D binding agent”, which can be a polypeptide or other molecule that interacts with PDE4D, such as a receptor) is contacted with PDE4D in the presence of a test agent, and the ability of the test agent to alter the interaction between PDE4D and the PDE4D binding agent is determined. Alternatively, a cell lysate or a solution containing the PDE4D binding agent, can be used. An agent which binds to PDE4D or the PDE4D binding agent can alter the interaction by interfering with, or enhancing the ability of PDE4D to bind to, associate with, or otherwise interact with the PDE4D binding agent. Determining the ability of the test agent to bind to PDE4D or an PDE4D binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with PDE4D or a PDE4D binding agent without the labeling of either the test agent, PDE4D, or the PDE4D binding agent. McConnell, H. M. et al. (1992) Science, 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide. See the Examples Section for a discussion of known PDE4D binding partners. Thus, these receptors can be used to screen for compounds that are PDE4D receptor agonists for use in treating stroke or PDE4D receptor antagonists for studying stroke. The linkage data provided herein, for the first time, provides such connection to stroke. Drugs could be designed to regulate PDE4D receptor activation that in turn can be used to regulate signaling pathways and transcription events of genes downstream, such as Cbfa1.

In another embodiment of the invention, assays can be used to identify polypeptides that interact with one or more PDE4D polypeptides, as described herein. For example, a yeast two-hybrid system such as that described by Fields and Song (Fields, S. and Song, O., Nature 340:245-246 (1989)) can be used to identify polypeptides that interact with one or more PDE4D polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also an PDE4D polypeptide, splicing variant, fragment or derivative thereof, and a second vector is used which includes a nucleic acid encoding a transcription activation domain and also a nucleic acid encoding a polypeptide which potentially may interact with the PDE4D polypeptide, splicing variant, or fragment or derivative thereof (e.g., a PDE4D polypeptide binding agent or receptor). Incubation of yeast containing the first vector and the second vector under appropriate conditions (e.g., mating conditions such as used in the Matchmaker™ System from Clontech) allows identification of colonies which express the markers of interest. These colonies can be examined to identify the polypeptide(s) that interact with the PDE4D polypeptide or fragment or derivative thereof. Such polypeptides may be useful as agents that alter the activity of expression of a PDE4D polypeptide, as described above.

In more than one embodiment of the above assay methods of the present invention, it may be desirable to immobilize either PDE4D, the PDE4D binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows PDE4D or a PDE4D binding agent to be bound to a matrix or other solid support.

In another embodiment, modulators of expression of nucleic acid molecules of the invention are identified in a method wherein a cell, cell lysate, or solution containing a nucleic acid encoding PDE4D is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a polypeptide encoded by PDE4D, or to alter expression of PDE4D, by contacting the polypeptide or the gene (or contacting a cell comprising the polypeptide or the gene) with the agent identified as described herein.

Pharmaceutical Compositions

The present invention also pertains to pharmaceutical compositions comprising agents described herein, particularly nucleotides encoding the polypeptides described herein; comprising polypeptides described herein (e.g., one or more of SEQ ID NO: 2, 3, 4, 5, 6, 7, 8, 9, 10, 12 or 14); and/or comprising other splicing variants encoded by PDE4D; and/or an agent that alters (e.g., enhances or inhibits) PDE4D gene expression or PDE4D polypeptide activity as described herein. For instance, a polypeptide, protein (e.g., an PDE4D receptor), an agent that alters PDE4D gene expression, or a PDE4D binding agent or binding partner, fragment, fusion protein or prodrug thereof, or a nucleotide or nucleic acid construct (vector) comprising a nucleotide of the present invention, or an agent that alters PDE4D polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of stroke, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention encompasses methods of treatment (prophylactic and/or therapeutic) for stroke or a susceptibility to stroke, such as individuals in the target populations described herein particularly ischemic (e.g., carotid and cardiogenic strokes) and TIA, using a PDE4D therapeutic agent. A “PDE4D therapeutic agent” is an agent that alters (e.g., enhances or inhibits) PDE4D polypeptide (enzymatic activity) and/or PDE4D gene expression, as described herein (e.g., a PDE4D agonist or antagonist). PDE4D therapeutic agents can alter PDE4D polypeptide activity or nucleic acid expression by a variety of means, such as, for example, by providing additional PDE4D polypeptide or by upregulating the transcription or translation of the PDE4D gene; by altering posttranslational processing of the PDE4D polypeptide; by altering transcription of PDE4D splicing variants; or by interfering with PDE4D polypeptide activity (e.g., by binding to a PDE4D polypeptide), or by downregulating the transcription or translation of the PDE4D gene.

In particular, the invention relates to methods of treatment for stroke or susceptibility to stroke (for example, for individuals in an at-risk population such as those described herein); as well as to methods of treatment for myocardial infarction, atherosclerosis, acute coronary syndrome (e.g., unstable angina, non-ST-elevation myocardial infarction (NSTEMI) or ST-elevation myocardial infarction (STEMI)); for decreasing risk of a second myocardial infarction; for atherosclerosis, such as for patients requiring treatment (e.g., angioplasty, stents, coronary artery bypass graft) to restore blood flow in arteries (e.g., coronary arteries) and peripheral arterial occlusive disease.

Representative PDE4D therapeutic agents include the following:
nucleic acids or fragments or derivatives thereof described herein, particularly nucleotides encoding the polypeptides described herein and vectors comprising such nucleic acids (e.g., a gene, cDNA, and/or mRNA, double-stranded interfering RNA, a nucleic acid encoding a PDE4D polypeptide or active fragment or derivative thereof, or an oligonucleotide; for example, SEQ ID NO: 1 which may optionally comprise at least one polymorphism shown in Tables 11 and 12 or a nucleic acid encoding SEQ ID NO: 2, 3, 4, 5, 6, 7, 8, 9, 10, 12 or 14, or fragments or derivatives thereof), antisense nucleic acids or small double-stranded interfering RNA;
polypeptides described herein (e.g., one or more of SEQ ID NO: 2, 3, 4, 5, 6, 7, 8, 9, 10, 12 or 14, and/or other splicing variants encoded by PDE4D, or fragments or derivatives thereof);
other polypeptides (e.g., PDE4D receptors); PDE4D binding agents; peptidomimetics; fusion proteins or prodrugs thereof; antibodies (e.g., an antibody to a mutant PDE4D polypeptide, or an antibody to a non-mutant PDE4D polypeptide, or an antibody to a particular splicing variant encoded by PDE4D, as described above); ribozymes; other small molecules;
and other agents that alter (e.g., inhibit or antagonize) PDE4D gene expression or polypeptide activity, or that regulate transcription of PDE4D splicing variants (e.g., agents that affect which splicing variants are expressed, or that affect the amount of each splicing variant that is expressed).

More than one PDE4D therapeutic agent can be used concurrently, if desired.

The PDE4D therapeutic agent that is a nucleic acid is used in the treatment of stroke. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition. In the case of atherosclerosis, “treatment” also refers to a minimization or reversal of the development of plaques. The therapy is designed to alter (e.g., inhibit or enhance), replace or supplement activity of a PDE4D polypeptide in an individual. For example, a PDE4D therapeutic agent can be administered in order to upregulate or increase the expression or availability of the PDE4D gene or of specific splicing variants of PDE4D, or, conversely, to downregulate or decrease the expression or availability of the PDE4D gene or specific splicing variants of PDE4D. Upregulation or increasing expression or availability of a native PDE4D gene or of a particular splicing variant could interfere with or compensate for the expression or activity of a defective gene or another splicing variant; downregulation or decreasing expression or availability of a native PDE4D gene or of a particular splicing variant could minimize the expression or activity of a defective gene or the particular splicing variant and thereby minimize the impact of the defective gene or the particular splicing variant.

The PDE4D therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a PDE4D polypeptide, such as SEQ ID NO: 1 which may optionally comprise at least one polymorphism shown in Tables 11 and 12; or another nucleic acid that encodes a PDE4D polypeptide or a splicing variant, derivative or fragment thereof, such as a nucleic acid encoding SEQ ID NO: 2, 3, 4, 5, 6, 7, 8, 9, 10, 12 or 14) can be used, either alone or in a pharmaceutical composition as described above. For example, PDE4D or a cDNA encoding the PDE4D polypeptide, either by itself or included within a vector, can be introduced into cells (either in vitro or in vivo) such that the cells produce native PDE4D polypeptide. If necessary, cells that have been transformed with the gene or cDNA or a vector comprising the gene or cDNA can be introduced (or re-introduced) into an individual affected with the disease. Thus, cells which, in nature, lack native PDE4D expression and activity, or have mutant PDE4D expression and activity, or have expression of a disease-associated PDE4D splicing variant, can be engineered to express PDE4D polypeptide or an active fragment of the PDE4D polypeptide (or a different variant of PDE4D polypeptide). In another embodiment, nucleic acid encoding the PDE4D polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of PDE4D is administered or generated in situ. The antisense nucleic acid that specifically hybridizes to the mRNA and/or DNA inhibits expression of the PDE4D polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA that encodes PDE4D polypeptide. Alternatively, the antisense construct can be an oligonucleotide probe that is generated ex vivo and introduced into cells; it then inhibits expression by hybridizing with the mRNA and/or genomic DNA of PDE4D. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ((1988) Cancer Res 48:2659-2668). With respect to antisense DNA, oligodeoxyribonucleotides derived from the translation initiation site, e.g., between the −10 and +10 regions of PDE4D sequence, are preferred.

To perform antisense therapy, oligonucleotides (mRNA, cDNA or DNA) are designed that are complementary to mRNA encoding PDE4D. The antisense oligonucleotides bind to PDE4D mRNA transcripts and prevent translation. Absolute complementarity, although preferred, is not required. a sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures. The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652; PCT International Publication No. WO88/09810) or the blood-brain barrier (see, e.g., PCT International Publication No. WO89/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide may be conjugated to another molecule (e.g., a peptide, hybridization triggered cross-linking agent, transport agent, hybridization-triggered cleavage agent).

The antisense molecules are delivered to cells that express PDE4D in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in another embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous PDE4D transcripts and thereby prevent translation of the PDE4D mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

Methods of modulating PDE4D expression by administering an RNA inhibitor of the activity of the target protein are also possible. The term “RNA inhibitor” refers to an inhibitory RNA that silences expression of the target protein by RNA interference (McManus, M. T. and Sharp, P. A., 2002. Nat. Rev. Genet. 3:737-47; Hannon, G. J., 2002. Nature 418:244-51; Paddison, P. J. and Hannon, G. J., 2002. Cancer Cell 2:17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 21 nucleotides, termed short-interfering RNAs or siRNAs, composed of 19 nucleotides of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

Recently, it was determined that gene silencing could be induced by presenting the cell with the siRNA, mimicking the product of Dicer cleavage (Elbashir, S. M., et al., 2001. Nature 411:494-8; Elbashir, S. M., et al., 2001. Genes Dev. 15:188-200). Synthetic siRNA duplexes maintain the ability to associate with RISC and direct silencing of mRNA transcripts, thus providing researchers with a powerful tool for gene silencing in mammalian cells. Yet another method to introduce the dsRNA for gene silencing is shRNA, for short hairpin RNA (Paddison, P. J., et al., 2002. Genes Dev. 16:948-58; Brummelkamp, T. R., et al., 2002 Science 296:550-3; Sui, G., et al., 2002. Proc. Natl. Acad. Sci. U.S.A. 99:5515-20). In this case, a desired siRNA sequence is expressed from a plasmid (or virus) containing an “shRNA” gene having an inverted repeat with an intervening loop sequence to form a hairpin structure. The resulting shRNA transcript containing the hairpin is subsequently processed by Dicer to produce siRNAs for silencing. Plasmid-based shRNAs can be expressed stably in cells, allowing long-term gene silencing in cells, or even in animals (McCaffrey, A. P., et al., 2002. Nature 418:38-9; Xia, H., et al., 2002. Nat. Biotech. 20:1006-10; Lewis, D. L., et al., 2002. Nat. Genetics 32:107-8; Rubinson, D. A., et al., 2003. Nat. Genetics 33:401-6; Tiscornia, G., et al., (2003) Proc. Natl. Acad. Sci. U.S.A. 100: 1844-8). RNA interference has been successfully used therapeutically to protect mice from fulminant hepatitis (Song, E., et al., 2003. Nat. Medicine 9:347-51).

Endogenous PDE4D expression can be also reduced by inactivating or “knocking out” PDE4D or its promoter using targeted homologous recombination (e.g., see Smithies et al. (1985) Nature 317:230-234; Thomas & Capecchi (1987) Cell 51:503-512; Thompson et al. (1989) Cell 5:313-321). For example, a mutant, non-functional PDE4D (or a completely unrelated DNA sequence) flanked by DNA homologous to the endogenous PDE4D (either the coding regions or regulatory regions of PDE4D) can be used, with or without a selectable marker and/or a negative selectable marker, to transfect cells that express PDE4D in vivo. Insertion of the DNA construct, via targeted homologous recombination, results in inactivation of PDE4D. The recombinant DNA constructs can be directly administered or targeted to the required site in vivo using appropriate vectors, as described above. Alternatively, expression of non-mutant PDE4D can be increased using a similar method: targeted homologous recombination can be used to insert a DNA construct comprising a non-mutant, functional PDE4D (e.g., a gene having SEQ ID NO: 1 which may optionally comprise at least one polymorphism shown in Tables 11 and 12), or a portion thereof, in place of a mutant PDE4D in the cell, as described above. In another embodiment, targeted homologous recombination can be used to insert a DNA construct comprising a nucleic acid that encodes a PDE4D polypeptide variant that differs from that present in the cell.

Alternatively, endogenous PDE4D expression can be reduced by targeting deoxyribonucleotide sequences complementary to the regulatory region of PDE4D (i.e., the PDE4D promoter and/or enhancers) to form triple helical structures that prevent transcription of PDE4D in target cells in the body. (See generally, Helene, C. (1991) Anticancer Drug Des., 6(6):569-84; Helene, C., et al. (1992) Ann, N.Y. Acad. Sci., 660:27-36; and Maher, L. J. (1992) Bioassays 14(12):807-15). Likewise, the antisense constructs described herein, by antagonizing the normal biological activity of one of the PDE4D proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a PDE4D mRNA or gene sequence) can be used to investigate role of PDE4D in developmental events, as well as the normal cellular function of PDE4D in adult tissue. Such techniques can be utilized in cell culture, but can also be used in the creation of transgenic animals.

In yet another embodiment of the invention, other PDE4D therapeutic agents as described herein can also be used in the treatment or prevention of stroke. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant PDE4D polypeptide in conjunction with antisense therapy targeting mutant PDE4D mRNA; administration of a first splicing variant encoded by PDE4D in conjunction with antisense therapy targeting a second splicing encoded by PDE4D), can also be used.

The invention will be further described by the following non-limiting examples. The teachings of all publications cited herein are incorporated herein by reference in their entirety.

### EXAMPLES

### Example 1

### PDE4D Variations and Haplotypes Increase Risk for Stroke

Icelandic Stroke Patients and Phenotype Characterization

A population-based list containing 2543 Icelandic stroke patients, diagnosed from 1993 through 1997, was derived from two major hospitals in Iceland and the Icelandic Heart Association (the study was approved by the Icelandic Data Protection Commission of Iceland and the National Bioethics Committee). Patients with hemorrhagic stroke represented 6% of all patients (patients with the Icelandic type of hereditary cerebral hemorrhage with amyloidosis and patients with subarachnoid hemorrhage were excluded). Ischemic stroke accounted for 67% of the total patients and TIAs 27%. The distribution of stroke suptypes in this study is similar to that reported in other Caucasian populations (Mohr, J. P., et al., Neurology, 28:754-762 (1978); L. R. Caplan, In Stroke, A Clinical Approach (Butterworth-Heinemann, Stoneham, Mass., ed 3, (1993)).

The list of approximately 2000 living patients was run through our computerized genealogy database. A comprehensive genealogy database that has been established at deCODE genetics was used to cluster the patients in pedigrees. Each version of the computerized genealogy database was reversibly encrypted by the Data Protection Commission of Iceland before arriving at the laboratory (Gulcher, J. R., et al., Eur. J. Hum. Genet. 8:739 (2000)). The database uses a patient list, with encrypted personal identifiers, as input, and recursive algorithms to find all ancestors in the database who are related to any member on the input list within a given number of generations back (Gulcher, J. R., and Stefansson, K., Clin. Chem. Lab. Med. 36:523 (1998)) covering the whole Icelandic nation. The cluster function then searches for ancestors who are common to any two or more members of the input list. One hundred and seventy-nine families with two or more living patients were chosen for the study with a total of 476 patients connected within 6 meioses (6 meioses connect second cousins). Informed consent was obtained from all patients and their relatives whose DNA samples were used in the linkage scan. The mean separation between affected pairs is 4.8 meioses. Of the patients selected for the study 73% had ischemic strokes, 23% TIAs and 4% hemorrhagic strokes.

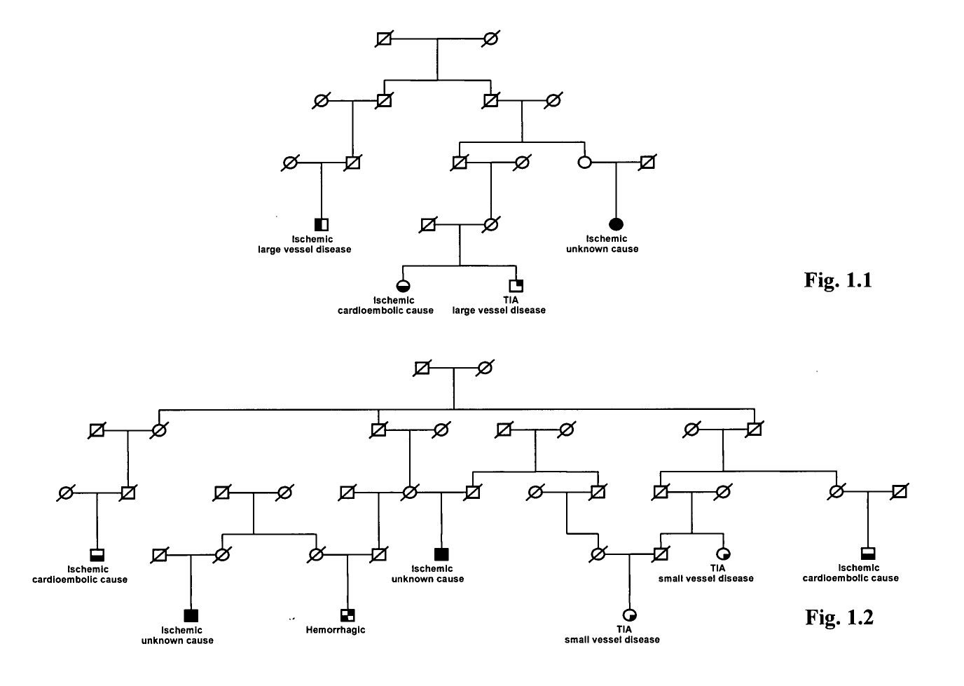

In the selected families, hemorrhagic stroke patients clustered with ischemic stroke and TIA patients, and there were no families with a striking preponderance of hemorrhagic stroke or of the subtypes of ischemic stroke. Patients with ischemic stroke were reclassified according to the TOAST (Trial of Org 10172 in Acute Stroke Treatment) sub-classification system for stroke (Adams, H. P., Jr., et al., Stroke, 24.34-41 (1993)). This system includes five categories: (1) large-artery atherosclerosis, (2) cardioembolism, (3) small-artery occlusion (lacune), (4) stroke of other determined etiology and (5) stroke of undetermined etiology. The diagnoses were based on clinical features and on data from ancillary diagnostic studies. Patients defined with large-artery atherosclerosis had clinical and brain imaging findings of cerebral cortical dysfunction and either significant (>70%) stenosis (this is a stricter criteria than used in TOAST where 50% stenosis is the cut-off) or occlusion of a major brain artery or branch cortical artery. Potential sources of cardiogenic embolism were excluded. The category cardioembolism included patients with at least one cardiac source for an embolus and potential large-artery sources of thromobosis and embolism was eliminated. Patients with small-artery occlusion had one of the traditional clinical lacunar syndromes and no evidence of cerebral cortical dysfunction. Potential cardiac source of embolus and stenosis >70% in an ipsilateral extracranial artery was excluded. The category, acute stroke of other determined etiology, included patients with rare causes of stroke and patients with two or more potential causes of stroke. If the causes of stroke could not be determined despite extensive evaluation patients were included in the category stroke of undetermined etiology. FIG. 1 displays two pedigrees each affected by several of the stroke subtypes, including hemorrhagic stroke. Apparently what is inherited in stroke is the broadly defined phenotype.

Genome-Wide Scan

A genome-wide scan was performed using a framework map of about 1000 microsatellite markers. The DNA samples were genotyped using approximately 1000 fluorescently labelled primers. A microsatellite screening set based in part on the ABI Linkage Marker (v2) screening set and the ABI Linkage Marker (v2) intercalating set in combination with 500 custom-made markers were developed. All markers were extensively tested for robustness, ease of scoring, and efficiency in 4× multiplex PCR reactions. In the framework marker set, the average spacing between markers was approximately 4 cM with no gaps larger than 10 cM. Marker positions were obtained from the Marshfield map, except for a three-marker putative inversion on chromosome 8 (Jonsdottir, G. M., et al., Am. J. Hum. Genet., 67 (Suppl. 2):332 (2000); Yu, A., et al., Am. J. Hum. Genet. 67 (Suppl. 2):10 (2000). The PCR amplifications were set up, run and pooled on Perkin Elmer/Applied Biosystems 877 Integrated Catalyst Thermocyclers with a similar protocol for each marker. The reaction volume used was 5 μl and for each PCR reaction 20 ng of genomic DNA was amplified in the presence of 2 pmol of each primer, 0.25 U AMPLITAQ GOLD (DNA polymerase; trademark of Roche Molecular Systems), 0.2 mM dNTPs and 2.5 mM MgCl2 (buffer was supplied by manufacturer). The PCR conditions used were 95° C. for 10 minutes, then 37 cycles of 15 s at 94° C., 30 s at 55° C. and 1 min at 72° C. The PCR products were supplemented with the internal size standard and the pools were separated and detected on Applied Biosystems model 377 Sequencer using v3.0 GENESCAN (peak calling software; trademark of Applied Biosystems). Alleles were called automatically with the TRUEALLELE (computer program for alleles identification; trademark of Cybergenetics, Inc.) program, and the program, DECODE-GT (computer editing program that works downstream of the TRUEALLELE program; trademark of deCODE genetics), was used to fractionate according to quality and edit the called genotypes (Palsson, B., et al., Genome Res. 9:1002 (1999)). At least 180 Icelandic controls were genotyped to derive allelic frequencies.

A total of 476 patients and 438 relatives were genotyped. The data was analyzed and the statistical significance determined by applying affecteds-only allele-sharing methods (which does not specify any particular inheritance model) implemented in the ALLEGRO (computer program for multipoint linkage analysis; trademark of deCODE genetics) program that calculates lod scores based on multipoint calculations. Our baseline linkage analysis uses the Sₚₐᵢᵣₛ scoring function (Kruglyak, L., et al., Am. J. Hum. Genet., 58:1347 (1996)), the exponential allele-sharing model (Kong, A. and Cox, N. J., Am. J. Hum. Genet., 61:1179 (1997)), and a family weighting scheme which is halfway, on the log scale, between weighting each affected pair equally and weighting each family equally. In the analysis we treat all genotyped individuals who are not affected as “unknown”. All linkage analyses in this paper were performed using multipoint calculation with the program ALLEGRO (deCODE genetics) (Gudbjartsson, D. F., et al., Nat. Genet. 25:12 (2000)).

The allele sharing lod scores for the genome scan using the framework map showed three regions that achieved a lod score above 1.0. Two of these regions are on chromosome 5q. The first peak is at approximately 69 cM with a lod score of 2.00. The second peak is at 99 cM with a lod score of 1.14. The third region is on chromosome 14q at 55 cM with a lod score of 1.24.

The information for linkage at the 5q locus was increased by genotyping an additional 45 markers over a 45 cM segment which spanned both peaks. The information used here is defined by Nicolae (D. L. Nicolae, Thesis, University of Chicago (1999)) and has been demonstrated to be asymptotically equivalent to a classical measure of the fraction of missing information (Dempster, A. P., et al., J R. Statist. Soc. B, 39:1 (1977)). While the lod score at the second peak dropped slightly to around 1.05, the lod score at the first peak increased to 3.39. However, close inspection of our results suggested that not only does the Marshfield genetic map lack resolution (many markers assigned the same map location), but also there may be some errors in their order. As a result, the genetic length of the region estimated using our material was substantially greater than what is reported. By modifying the ALLEGRO (deCODE genetics) program, we applied the EM algorithm to our data to estimate the genetic distances between markers. We found that our estimate of the genetic length of the region was substantially longer than that given in the Marshfield map. This indicates a problem with marker order because, in general, incorrect marker order leads to an increased number of apparent crossovers and increases the apparent genetic length.

Physical and Genetic Mapping

The marker order and inter-marker distances were improved by constructing high density physical and genetic maps over a 20 cM region between markers D5S474 and D5S2046. A combination of data from coincident hybridizations of BAC membranes using a high density of STSs and the Fingerprinting Contig database was used to build large contigs of BACs from the RPCI-11 library. The order of the linkage markers was also confirmed by high-resolution genetic mapping using the stroke families supplemented with over 112 other large nuclear families. High resolution genetic mapping was used both to anchor and place in order contigs found by physical mapping as well as to obtain accurate inter-marker distances for the correctly ordered markers. Data from 112 Icelandic nuclear families (sibships with their parents, containing from two to seven siblings) were analyzed together with the nuclear families available within the stroke pedigrees. For the purpose of genetic mapping the 112 nuclear families alone provide 588 meioses, and the total number of meioses available for mapping was over 2000. By comparison, the Marshfield genetic map was constructed based on 182 meioses. The large number of meiotic events within our families provides the ability to map markers to the resolution of 0.5 to 1.0 cM. Combining this information with the physical map resulted in a highly reliable order of markers and inter-marker distances within this 20 cM region. Linkage markers common to the genetic and physical maps were used to anchor and place in order four of the physically mapped contigs. By integrating the genetic and physical maps a most likely order of 30 polymorphic markers was derived.

BAC contigs were generated by a method that combines coincident primer hybridization with data mining. The RPCI-11 human male BAC library segments 1 & 2 (Pieter de Jong, Children's Hospital Oakland Research Institute) containing about 200,000 clones with a 12× coverage, were gridded using a 6×6 double offset pattern in 23 cm×23 cm membranes with a BioGrid robot (Biorobotics Ltd., Cambridge, UK). Initially, hybridizations were performed with markers in the region of interest according to their location in the Weizmann Institute Unified Database. Primer sequences were analyzed and discarded according to their content of known repeats, E. Coli and vector sequences (the analysis was performed using software developed at deCODE genetics). One hundred and fifty markers in the region (30 polymorphic markers used in linkage and 120 generated from STSs) separated by an average of 130 kb were used. The selected markers were used to generate two ³²P labelled probes, F that contained the pooled forward primers and R that contained the pooled reverse primers. Reading of positive signals was performed automatically from digitized images of resulting autoradiograms by informatics tools developed at deCODE genetics. The coincident signals in both hybridizations were selected as positive clones. A set of overlapping clones was assembled through a combination of hybridization and BAC fingerprint walking. Fingerprints of positive clones were analyzed using the FPC database developed at the Sanger Center. Data from FPC contigs prebuilt with a cutoff of 3e-12 and from sequence datamining was integrated with the hybridization results. BACs in the region detected by data mining and hybridization were re-arrayed using a Multiprobe IIex robot (Packard, Meriden, Conn.). Small membranes (8 cm×12 cm) were gridded in 6×6 double offset pattern and individually hybridized with the markers of interest. Positive patterns were transferred using transparencies to an Excel file containing macros to provide BAC to marker associations. A visual map was generated by combining the hybridization, fingerprinting and sequence data. New markers were generated from BAC end sequences to close the gap. After several rounds of hybridization positive BACs were assembled into 7 contigs covering approximately 20 Mb. Thirty of the polymorphic markers used in linkage were assigned to four of the contigs. Estimation of contig lengths and distance between markers assigned to them was based on the FPC program.

Twenty-seven of our 30 linkage markers mapped to three contigs in the October 2000 release from UCSC, the UC Santa Cruz (UCSC) draft assembly. (http://genome.ucsc.edu/). The marker order within the contigs is in agreement with our order with the exception of two markers. Although the UCSC assemblies are improving, some contigs have incorrect order, orientation, or contig assembly. We believe that high resolution genetic mapping and perhaps focused hybridization experiments are still necessary to confirm accuracy of sequence assemblies. In addition, high resolution genetic mapping provides better estimates of inter-marker genetic distances that are also important for linkage analysis (Halpern, J. and Whittermore, A. S., Hum. Hered. 49:194 (1999); Daw, E. W., et al., Genet. Epidemiol. 19:366 (2000)).

Statistical Methods for Linkage Analysis

Multipoint, affected-only allele-sharing methods were used in the analyses to assess evidence for linkage. All results, both the LOD-score and the non-parametric linkage (NPL) score, were obtained using the program Allegro (Gudbjartsson et al., Nat. Genet. 25:12-3, 2000). Our baseline linkage analysis, as previously described (Gretarsdottir et al., Am J Hom Genet, 70:593-603, 2002), uses the Sₚₐᵢᵣₛ scoring function (Whittemore, A. S., Halpern, J. (1994), Biometrics 50:118-27; Kruglyak L, et al. (1996), Am J Hum Genet 58:1347-63), the exponential allele-sharing model (Kong, A. and Cox, N. J. (1997), Am J Hum Genet 61:1179-88) and a family weighting scheme that is halfway, on the log-scale, between weighting each affected pair equally and weighting each family equally. The information measure we use is part of the Allegro program output and the information value equals zero if the marker genotypes are completely uninformative and equals one if the genotypes determine the exact amount of allele sharing by decent among the affected relatives (Gretarsdottir et al., Am. J. Hom. Genet, 70:593-603, (2002)). We computed the P-values two different ways and here report the less significant result. The first P-value was computed on the basis of large sample theory; the distribution of Zᵢᵣ=√(2[logₑ(10)LOD]) approximates a standard normal variable under the null hypothesis of no linkage (Kong, A. and Cox, N. J. (1997), Am J Hum Genet 61:1179-88). The second P-value was calculated by comparing the observed LOD-score with its complete data sampling distribution under the null hypothesis (Gudbjartsson et al., Nat. Genet. 25:12-3, 2000). When the data consist of more than a few families, as is the case here, these two P-values tend to be very similar.

Final Linkage Results and Localization

Linkage analysis including genotypes from the higher density markers using the deCODE marker order resulted in a lod score of 4.40 (P=3.9×10⁻⁶) on chromosome 5q12 at the marker D5S2080. The reported P value is part of the output of the ALLEGRO (deCODE genetics) program which was developed at deCODE and has become a standard linkage program worldwide over the last 3 years (Gudbjartsson et al., Nat. Genet. 25:12-3, 2000). We have given it to over 200 academic departments around the world free of charge and it is widely used. The locus has been designated as STRK1. With the addition of these extra markers, it was possible to narrow down the region to a segment less than 6 cM, from D5S1474 to D5S398, as defined by one drop in lod.

To further investigate the contribution of this susceptibility locus to stroke, a range of parametric models were fitted to the data. However, all analyses were still affecteds only in the sense that individuals were either classified as affecteds or having unknown disease status. A lod score of 4.08 was obtained with a dominant model where the allele frequency of the susceptibility gene was assumed to be 5% and carriers of the alteration were assumed to have seven-fold the risk of a non-carrier. By inspecting the individual families, no obvious correlation was seen between families that contribute positively to the linkage results with the prevalence of hypertension, diabetes or hyperlipidemias. When the data were reanalyzed with the hemorrhagic stroke patients removed, the allele sharing lod score increased to 4.86 at D5S2080. Although this 0.46 increase in log score suggests that STRK1 is involved primarily in ischemic stroke and TIAs, it is not statistically significant based on simulations (one sided P equals 0.09). In order to assess whether such a change in lod score would be likely to occur by chance we selected 1000 random sets of 22 patients whose status we then changed to “unknown” in an analysis. The P value we present is the fraction of the 1000 simulations which produce a lod score increase at the peak locus equal to or greater than that which we observed by changing the affection status of the 22 hemorrhagic stroke patients to “unknown”.

Identification of Allelic Association

All microsatellite markers in the approx. 6 cM interval (markers from D5S398 to D5S1474) were analyzed with respect to allelic association.

Microsatellite Allelic Association

We initially genotyped 864 Icelandic stroke patients and 908 controls using a total of 98 microsatellite markers. These markers are distributed over a region of approximately 11 Mb. The region is centered on our linkage peak and corresponds to the 2 LOD drop. The density of markers is greater in the central 3.7 Mb portion of the region, which includes the 1 LOD drop, with an average spacing of one marker every 53 kb. We have designated this central region, which is flanked by markers D5S 1474 and D5S398, as the STRK1 interval. Three markers, AC027322-5, D5S2121 and AC008818-1, showed a difference in allelic frequency between patients and controls with p-values less than 0.01 (Table 1). Correcting for the relatedness of the Icelandic patients had little impact on the p-values, but after correcting for the number of markers and alleles tested none of these p-values were significant (Table 1).

We had previously observed that our linkage peak increased, albeit not significantly, when excluding the hemorrhagic stroke patients. We therefore tested only those patients with ischemic stroke or TIA for association to the markers. In addition, our ischemic stroke and TIA patients have been sub-classified according to the TOAST research criteria and we also repeated the association analysis separately for patients with the three TOAST subcategories: cardiogenic, carotid (greater than 70% stenosis) and small vessel occlusive disease. Lastly, we tested the combination of patients with cardiogenic and carotid stroke, since these categories of stroke are most clearly related to atherosclerosis. The results for each of these association studies are presented in Table 1. Three of the tests, one for cardiogenic stroke (AC008818-1), one for carotid stroke (DG5S397), and one for the combination of carotid and cardiogenic stroke (AC008818-1) were significant even after correcting for multiple testing (Table 1). The marker DG5S397 is located within the PDE4D gene and AC008818-1 is in the 5′ end of PDE4D and in the overlapping gene Prostate androgen-regulated transcript (PART1) whose transcript is on the other strand going in the opposite direction. PDE4D is an important regulator of intracellular levels of cAMP and is expressed widely. PART1 encodes a putative protein with unknown function predominantly expressed in the prostate gland and in several cancer cell lines. Physical locations of all genotyped markers and PDE4D and PART1 exons are available in Table 2C. The association results for the combination of carotid and cardiogenic stroke were particularly striking with an allele frequency of 35.5% in patients for allele 0 (the CEPH reference allele) of marker AC008818-1 versus 25.5% in controls. The unadjusted p-value for this marker is 0.0000015, and after adjusting for multiple testing of markers is 0.00025 (Table 1). This remains significant even after adjusting for the several phenotypes studied. The risk of this allele to the other alleles of this marker, assuming the multiplicative model Terwilliger, J. D. & Ott, J. A haplotype-based ‘haplotype relative risk’ approach to detecting allelic associations. Hum Hered 42, 337-46 (1992) and Falk, C. T. & Rubinstein, P. Haplotype relative risks: an easy reliable way to construct a proper control sample for risk calculations. Ann Hum Genet 51 (Pt 3), 227-33 (1987), was estimated to be 1.60, and the corresponding population attributable risk was 25%.

Thus, the strong association signals from our initial microsatellite association studies helped to focus our attention on the STRK1 interval and, in particular, to the PDE4D gene region.

TABLE 1
Microsatellite allelic association analysis of the two-lod drop of the STRK1 locus.
All microsatellites that show association with a p-value less than 0.01 for all stroke, all stroke
excluding hemorrhagic stroke, cardiogenic stroke, carotid stroke, small vessel disease and the
combination of cardio genic and carotid stroke *significant after adjusting for multiple testing

| Phenotype | Marker | Allele | p-value | RR | # Aff. | Aff. % | # Ctrl | Ctrl. % |
|---|---|---|---|---|---|---|---|---|
| All | AC027322-5 | 10 | 0.001 | 3.34 | 787 | 1.90 | 779 | 0.6 |
| | D5S2121 | −2 | 0.0027 | 2.19 | 824 | 2.7 | 870 | 1.3 |
| | AC008818-1 | 0 | 0.0045 | 1.25 | 815 | 29.9 | 891 | 25.5 |
| All patients - | AC027322-5 | 10 | 0.00052 | 3.56 | 740 | 20 | 779 | 0.6 |
| excluding | D5S2121 | −2 | 0.0023 | 2.23 | 774 | 2.8 | 870 | 1.3 |
| hemorrhagic | AC008818-1 | 0 | 0.0062 | 1.24 | 764 | 29.9 | 891 | 25.5 |
| stroke | | | | | | | | |
| Cardiogenic | AC008818-1 | 0 | 0.000054* | 1.60 | 216 | 35.4 | 891 | 25.5 |
| stroke | D5S1990 | 20 | 0.00053 | 2.18 | 223 | 7.9 | 879 | 3.8 |
| | D5S2089 | −10 | 0.0027 | 2.22 | 219 | 5.9 | 813 | 2.8 |
| | D5S1359 | 2 | 0.0044 | 1.39 | 214 | 36.0 | 777 | 28.8 |
| | AC016604-2 | 0 | 0.0048 | 1.44 | 170 | 51.8 | 446 | 42.7 |
| | AC008804-1 | 0 | 0.0068 | 1.52 | 128 | 36.3 | 367 | 27.3 |
| | AC022125-3 | 0 | 0.0077 | 1.36 | 223 | 36.8 | 775 | 30.0 |
| | DG5S2066 | 0 | 0.0095 | 1.80 | 166 | 92.5 | 501 | 87.2 |
| | DG5S2039 | 9 | 0.0084 | 2.00 | 167 | 8.7 | 491 | 4.6 |
| | D5S647 | −6 | 0.0091 | 2.43 | 199 | 3.8 | 789 | 1.6 |
| Carotid stroke | DG5S397 | 4 | 0.00024* | 1.70 | 124 | 65.7 | 577 | 53.0 |
| | DG5S2056 | 12 | 0.0009 | 3.33 | 80 | 8.8 | 464 | 2.8 |
| | AC008818-1 | 0 | 0.001 | 1.61 | 125 | 35.6 | 891 | 25.5 |
| | DG5S2039 | −3 | 0.003 | 1.62 | 96 | 45.8 | 491 | 34.3 |
| | DG5S2045 | 0 | 0.0051 | 1.80 | 55 | 57.3 | 339 | 42.6 |
| | DG5S818 | 6 | 0.0079 | 1.50 | 111 | 63.1 | 563 | 53.3 |
| | AC016604-3 | 4 | 0.0072 | 1.53 | 99 | 40.9 | 645 | 31.2 |
| Small vessel | D5S1359 | 2 | 0.0085 | 1.41 | 157 | 36.3 | 777 | 28.8 |
| disease | D5S2080 | 2 | 0.0092 | 1.38 | 153 | 54.6 | 885 | 46.5 |
| | D5S2121 | −2 | 0.0059 | 2.93 | 152 | 3.6 | 870 | 1.3 |
| Combined | AC008818-1 | 0 | 0.0000015* | 1.60 | 341 | 35.5 | 891 | 25.5 |
| cardiogenic & | AC008833-6 | 0 | 0.0026 | 1.35 | 335 | 70.3 | 868 | 63.8 |
| carotid stroke | DG5S2066 | 0 | 0.0032 | 1.74 | 258 | 92.3 | 501 | 87.2 |
| | DG5S397 | 4 | 0.009 | 1.29 | 345 | 59.3 | 577 | 53.0 |
| | D5S2121 | −2 | 0.0081 | 2.39 | 336 | 3.0 | 870 | 1.3 |
| Alleles #'s: For SNP alleles A = 0, C = 1, G = 2, T = 3; for microsatellite alleles: the CEPH sample 1347-02 (CEPH genomics repository) is used as a reference, the lower allele of each microsatellite in this sample is set at 0 and all other alleles in other samples are numbered | | | | | | | | |
| # accordingly in relation to this reference. Thus allele 1 is 1 bp longer than the lower allele in the CEPH sample 1347-02, allele 2 is 2 bp longer than the lower allele in the CEPH sample 1347- 02, allele 3 is 3 bp longer than the lower allele in the CEPH sample 1347-02, allele 4 is 4 bp | | | | | | | | |
| # longer than the lower allele in the CEPH sample 1347-02, allele −1 is 1 bp shorter than the lower allele in the CEPH sample 1347-02, allele −2 is 2 bp shorter than the lower allele in the EPH sample 1347-02, and so on. Note that this same CEPH sample is a standard that is widely used | | | | | | | | |
| # throughout the world for calibration and comparison of alleles. | | | | | | | | |

AC008818-1 amplimer:

(SEQ ID NO: 86)

TGCTTGGTGAAGGAATAGCCACCCCAGAGAAGGAGTATGGACTTCTATAC
ACAATCATTCATTCATTCATTCATTCATTCATTCATTCATTCATTCACTA
CTCATGCATGATCTTTGTCCTTATCTTCCTCCACTGTCACATGAATACCC
ACCCACTGCACCTACCTGCTTCCTATTCCTGAGAACCCAGGCTC

AC008818-1, allele 0 is the same allele as the minimum allele observed in CEPH 1347-02, family 137, individual 02.

Swedish patients have also been genotyped and microsatellite single and multimarker association has been analyzed using the E-M algorithm. A total number of 943 Swedish patients (stroke patients and patients with carotid stenosis) and 322 Swedish controls were analyzed (results shown in Table 2A). At least three haplotypes were more common in patients compared to controls, confirming in a second population that PDE4D shows association to stroke.

TABLE 2A
Swedish Patient Association

| Markers | Alleles | pAllelic | All Frq Aff | All Frq Ctrl | # aff | # ctrl |
|---|---|---|---|---|---|---|
| Swedish patients (n = 943) | | | | | | |
| D5S2000 | 2 | 0.0024 | | | 912 | 318 |
| (Sw 2) AC022125-3 | 0 0 2 0 | 0.006 | 0.035 | 0.01 | 717 | 284 |
| AC008833-6 D5S2000 D5S2091 | | | | | | |
| (Sw-1) AC008804-2 D17-H | −2 4 −2 10 | 0.0028 | 0.057 | 0.05 | 672 | 113 |
| D17-G D5S2080 | | | | | | |
| AC008804-2 D17-H D17-G | −4 0 −2 | 0.0037 | 0.056 | 0.03 | 700 | 123 |

Screening for Polymorphisms in PDE4D

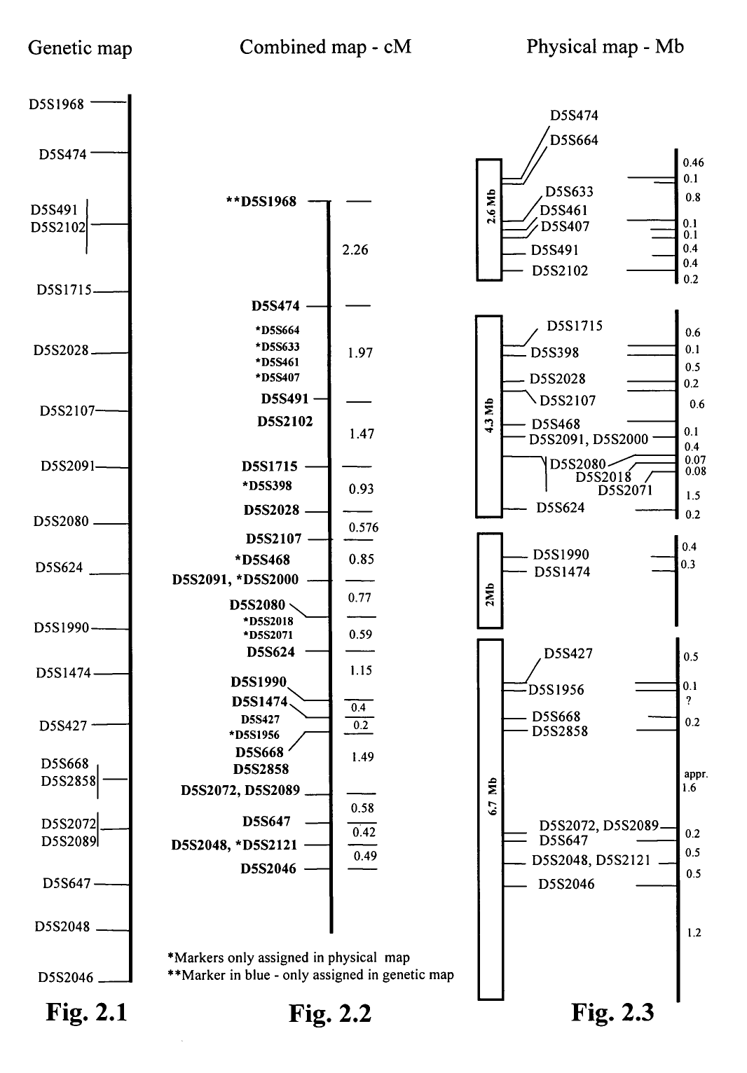

We next considered whether a functional variant in the PDE4D gene might be the cause of our observed microsatellite association. We matched public domain ESTs and our own RT-PCR and RACE transcripts to our sequence of the STRK1 interval. We defined new alternative PDE4D transcripts, which together with previously known transcripts indicated that the PDE4D gene contains 22 exons over at least 1.5 Mb and overlaps with PART1. The PDE4D gene encodes eight protein isoforms and has at least seven promoters. All isoforms identified have an identical C-terminal catalytic domain but differ at the N-terminal regulatory domain (FIG. 2).

We then attempted to identify mutations by sequencing all known PDE4D exons (including the overlapping PART1 exons) and, on average, 100 bp of their flanking introns in 188 patients and 94 controls. Forty-six polymorphisms were identified; 44 SNPs and two intronic deletions. Only two of the polymorphisms, both SNPs, were found within the coding exons of the PDE4D gene, which is consistent with the extraordinary lack of variation that others have reported for all four PDE4 classes Houslay, M. D. & Adams, D. R. PDE4 cAMP phosphodiesterases: modular enzymes that orchestrate signalling cross-talk, desensitization and compartmentalization. Biochem J 370, 1-18 (2003). The two coding SNPs were typed for additional patients and controls. However, these SNPs did not show significant association to stroke (Table 2B). Therefore, if a functional variant conferring risk for stroke exists in the PDE4D gene, it may be within regulatory regions affecting transcription, splicing, message stability, or message transport of one or more isoforms, or in exons that we have not yet identified.

TABLE 2B
Frequency of PDE4D coding mutations.

| | AA | PDE4D | | p- | Aff. | Ctrl. | | |
|---|---|---|---|---|---|---|---|---|
| Markers | change | exon | Allele | value | % | % | # Aff | # Ctrl |
| SNP | Pro > | D1/D2 | A | 0.163 | 2.0 | 1.5 | 604 | 369 |
| 250 | Thr | | | | | | | |
| SNP | Lys > | 4 | C | 0.381 | 0.2 | 0.0 | 474 | 294 |
| 257 | Thr | | | | | | | |

PDE4D Isoform Expression

Failing to find a functional mutation in the known coding exons of PDE4D, we were interested to consider other possible evidence in favor of this gene being a source of the underlying association in this region. We conducted an experiment to study the expression levels of the various isoforms—with any significant differences between patients and controls potentially indicating that regulation of PDE4D is a key element in stroke susceptibility. We used EBV transformed B cell lines from randomly selected patients having ischemic stroke or TIA and from controls. We carried out isoform-specific kinetic RT-PCR analysis to quantify each isoform in 83 stroke patients and 84 controls. The patients were principally ischemic stroke patients, with 32 of them having cardiogenic or carotid stroke. We observed that the total PDE4D message level, as assessed by amplification across exons present in all isoforms (PAN), was significantly lower in patients than in controls (p-value=0.0021). This decrease was due primarily to lower expression of the PDE4D1, PDE4D2 and PDE4D5 isoforms. This significant disregulation of the expression of multiple PDE4D isoforms greatly encouraged us to continue our investigations into the association of the PDE4D gene to stroke.

TABLE 2C
SNP identification, single marker association and LD mapping of the PDE4D
region

| SNP | | Public | start in NCBI | end in NCBI | start in SEQ | end in SEQ |
|---|---|---|---|---|---|---|
| code | marker or exon | name | build 31 | build 33 | ID NO: 1 | ID NO: 1 |
| | AC016604 - 3 | | 57547045 | 57547304 | | |
| | AC016604 - 2 | | 57623148 | 57623287 | | |
| | exon 11 | | 58241020 | 58241432 | 1655335 | 1655747 |
| | exon 10 | | 58242009 | 58242191 | 1654576 | 1654758 |
| | exon 9 | | 58242702 | 58242824 | 1653943 | 1654065 |
| | exon 8 | | 58243543 | 58243697 | 1653070 | 1653224 |
| | exon 7 | | 58254845 | 58254944 | 1641818 | 1641917 |
| | exon 6 | | 58256107 | 58256271 | 1640491 | 1640655 |
| | exon 5 | | 58257156 | 58257254 | 1639508 | 1639606 |
| | exon 4 | | 58258185 | 58258356 | 1638406 | 1638578 |
| | exon 3 | | 58259724 | 58259817 | 1636944 | 1637037 |
| | exon D1/D2 | | 58305211 | 58305581 | 1591172 | 1591425 |
| | exon LF4 | | 58446946 | 58446995 | 1449835 | 1449884 |
| | exon LF3 | | 58451540 | 58451613 | 1445217 | 1445290 |
| | exon LF2 | | 58459851 | 58459887 | 1436943 | 1436979 |
| | exon LF1 | | 58482128 | 58482319 | 1414511 | 1414702 |
| | AC022125 - 3 | | 58504109 | 58504274 | 1392556 | 1392721 |
| SNP 204 | SNP5PD890407 | | 58506423 | 58506423 | 1390407 | 1390407 |
| | AC008833 - 6 | | 58507019 | 58507222 | 1389608 | 1389811 |
| | exon D9 | | 58541689 | 58542470 | 1354347 | 1355128 |
| | D5S2000 | | 58585460 | 58585849 | | |
| | D5S2091 | | 58593284 | 58593634 | | |
| | exon D8 | | 58623109 | 58623414 | 1273404 | 1273709 |
| | D17 - C | | 58645088 | 58645386 | 1251432 | 1251730 |
| | AC008804 - 1 | | 58784449 | 58784641 | 1112181 | 1112373 |
| | AC008804 - 2 | | 58817743 | 58817931 | 1078881 | 1079069 |
| | exon D3 | | 58852680 | 58852819 | 1044051 | 1044190 |
| | D17 - H | | 58860588 | 58860725 | 1036142 | 1036279 |
| | D17 - G | | 58942270 | 58942541 | 954298 | 954569 |
| | D5S2080 | | 58998685 | 58999021 | | |
| | exon D5 | | 59034598 | 59035009 | 861791 | 862202 |
| | AC027322 - 5 | | 59159221 | 59159326 | 737420 | 737519 |
| | exon D4 | | 59159520 | 59160492 | 736254 | 737226 |
| SNP 102 | SNP5PD166822 | rs714291 | 59229897 | 59229897 | | |
| | exon D7 - 3 | | 59254840 | 59255069 | 641649 | 641878 |
| SNP 101 | SNP5PD138604 | rs1347401 | 59258113 | 59258113 | 638605 | 638605 |
| SNP 100 | SNP5PD121753 | rs1545070 | 59274962 | 59274962 | | |
| SNP 99 | SNP5PD118378 | rs1533019 | 59278338 | 59278338 | | |
| SNP 98 | SNP5PD117029 | rs952110 | 59279687 | 59279687 | | |
| SNP 97 | SNP5PD104361 | rs1995780 | 59292356 | 59292356 | | |
| SNP 96 | SNP5PD97409 | | 59299308 | 59299308 | | |
| SNP 95 | SNP5PD97281 | rs2016324 | 59299437 | 59299437 | | |
| SNP 94 | SNP5PD75406 | rs1396474 | 59321313 | 59321313 | | |
| SNP 93 | SNP5PD73383 | rs1508864 | 59323336 | 59323336 | | |
| SNP 92 | SNP5PD72097 | rs1508859 | 59324622 | 59324622 | | |
| | DG5S2045 | | 59325313 | 59325563 | 571152 | 571406 |
| | DG5S2039 | | 59332799 | 59333077 | 563636 | 563921 |
| SNP 91 | SNP5PD46864 | rs1508863 | 59349851 | 59349851 | | |
| SNP 90 | SNP5PD43868 | rs2136203 | 59352849 | 59352849 | | |
| SNP 89 | SNP5PD29517 | rs1396476 | 59367167 | 59367167 | | |
| | DG5S2056 | | 59381102 | 59381367 | 515317 | 515582 |
| | DG5S818 | | 59384776 | 59384999 | 511685 | 511908 |
| SNP 88 | SNP5PDM14337 | rs1544788 | 59411021 | 59411021 | | |
| | DG5S397 | | 59438506 | 59438784 | 457900 | 458178 |
| SNP 87 | SNP5PDM43741 | rs2910829 | 59440424 | 59440424 | | |
| | exon D7 - 2 | | 59451909 | 59452039 | 444645 | 444775 |
| SNP 86 | SNP5PDM57997 | rs2962972 | 59454680 | 59454680 | | |
| SNP 85 | SNP5PDM65461 | rs2961897 | 59462144 | 59462144 | | |
| SNP 84 | SNP5PDM67604 | rs719702 | 59464287 | 59464287 | | |
| SNP 83 | SNP5PDM76361 | rs966221 | 59473045 | 59473045 | | |
| SNP 82 | SNP5PDM83539 | rs2961903 | 59480223 | 59480223 | | |
| SNP 81 | SNP5PDM89176 | | 59485859 | 59485859 | 410826 | 410826 |
| SNP 80 | SNP5PDM89683 | rs1862614 | 59486368 | 59486368 | | |
| | DG5S2066 | | 59522085 | 59522346 | 374339 | 374600 |
| SNP 79 | SNP5PDM132154 | | 59528838 | 59528838 | 367847 | 367847 |
| SNP 78 | SNP5PDM153120 | | 59549804 | 59549804 | 346881 | 346881 |
| SNP 77 | SNP5PDM161561 | | 59558245 | 59558245 | 338440 | 338440 |
| SNP 76 | SNP5PDM166786 | | 59563470 | 59563470 | 333215 | 333215 |
| SNP 75 | SNP5PDM181173 | | 59577856 | 59577856 | 318829 | 318829 |
| SNP 74 | SNP5PDM182792 | | 59579475 | 59579475 | 317210 | 317210 |
| SNP 73 | SNP5PDM211974 | | 59608650 | 59608650 | 288027 | 288027 |
| SNP 72 | SNP5PDM217886 | | 59614557 | 59614557 | 282115 | 282115 |
| SNP 71 | SNP5PDM218639 | | 59615310 | 59615310 | 281362 | 281362 |
| SNP 70 | SNP5PDM224528 | | 59621190 | 59621190 | 275473 | 275473 |
| SNP 69 | SNP5PDM236461 | rs1423248 | 59633124 | 59633124 | | |
| SNP 68 | SNP5PDM259844 | | 59656504 | 59656504 | 240157 | 240157 |
| SNP 67 | SNP5PDM261488 | | 59658148 | 59658148 | 238513 | 238513 |
| SNP 66 | SNP5PDM265669 | | 59662328 | 59662328 | 234332 | 234332 |
| SNP 65 | SNP5PDM271674 | rs918590 | 59668333 | 59668333 | | |
| SNP 64 | SNP5PDM275805 | rs1423247 | 59672463 | 59672463 | | |
| SNP 63 | SNP5PDM280894 | rs789389 | 59677551 | 59677551 | | |
| SNP 62 | SNP5PDM285592 | | 59682247 | 59682247 | 214409 | 214409 |
| SNP 61 | SNP5PDM296955 | rs37691 | 59693610 | 59693610 | | |
| SNP 60 | SNP5PDM299842 | | 59696497 | 59696497 | 200159 | 200159 |
| SNP 59 | SNP5PDM307243 | rs37684 | 59703890 | 59703890 | | |
| SNP 58 | SNP5PDM308509 | rs2898278 | 59705155 | 59705155 | | |
| SNP 57 | SNP5PDM310220 | rs401207 | 59706866 | 59706866 | | |
| SNP 56 | SNP5PDM310653 | rs702553 | 59707298 | 59707298 | | |
| SNP 55 | SNP5PDM324741 | rs251726 | 59721387 | 59721387 | | |
| SNP 54 | SNP5PDM326519 | rs27223 | 59723165 | 59723165 | | |
| SNP 53 | SNP5PDM329913 | | 59726556 | 59726556 | 170088 | 170088 |
| SNP 52 | SNP5PDM332989 | | 59729632 | 59729632 | 166900 | 166900 |
| SNP 51 | SNP5PDM338487 | | 59735122 | 59735122 | 161514 | 161514 |
| SNP 50 | SNP5PDM345627 | rs173591 | 59742248 | 59742248 | | |
| SNP 49 | SNP5PDM349039 | rs27220 | 59745661 | 59745661 | | |
| SNP 48 | SNP5PDM351840 | rs37760 | 59748461 | 59748461 | | |
| SNP 47 | SNP5PDM356081 | | 59752701 | 59752701 | 143922 | 143922 |
| SNP 46 | SNP5PDM356447 | | 59753067 | 59753067 | 143555 | 143555 |
| SNP 45 | SNP5PDM357221 | | 59753842 | 59753842 | 142780 | 142780 |
| SNP 44 | SNP5PDM357245 | | 59753865 | 59753865 | 142757 | 142757 |
| SNP 43 | SNP5PDM357445 | | 59754066 | 59754066 | 142556 | 142556 |
| | PART1-exon 1 | | 59754284 | 59754775 | | |
| | exon D7-1 | | 59754294 | 59754415 | 142207 | 142328 |
| | PART1-exon 2 | | 59756013 | 59757617 | | |
| SNP 42 | SNP5PDM361194 | rs153031 | 59757816 | 59757816 | | |
| SNP 41 | SNP5PDM361545 | | 59758341 | 59758341 | 138456 | 138456 |
| | AC008818-1 | SEQ ID | 59759882 | 59760075 | 136740 | 136547 |
| | | NO: 86 | | | | |
| SNP 40 | SNP5PDM363736 | | 59760357 | 59760357 | 136265 | 136265 |
| SNP 39 | SNP5PDM364360 | rs3887175 | 59760981 | 59760981 | | |
| SNP 38 | SNP5PDM364848 | | 59761469 | 59761469 | 135152 | 135152 |
| SNP 37 | SNP5PDM364888 | rs26956 | 59761510 | 59761510 | 135112 | 135112 |
| SNP 36 | SNP5PDM366629 | | 59763250 | 59763250 | 133371 | 133371 |
| SNP 35 | SNP5PDM367438 | rs26955 | 59764060 | 59764060 | 132562 | 132562 |
| SNP 34 | SNP5PDM368135 | rs27653 | 59764755 | 59764755 | 131865 | 131865 |
| SNP 33 | SNP5PDM369610 | | 59766229 | 59766229 | 130391 | 130391 |
| SNP 32 | SNP5PDM370640 | | 59767259 | 59767259 | 129361 | 129361 |
| SNP 31 | SNP5PDM370641 | rs457053 | 59767260 | 59767261 | 129360 | 129360 |
| SNP 30 | SNP5PDM374696 | rs27221 | 59771316 | 59771316 | 125304 | 125304 |
| SNP 29 | SNP5PDM376181 | rs2963110 | 59772800 | 59772800 | | |
| SNP 28 | SNP5PDM376575 | rs35387 | 59773194 | 59773194 | 123426 | 123426 |
| SNP 27 | SNP5PDM376688 | rs35386 | 59773308 | 59773308 | 123312 | 123312 |
| SNP 26 | SNP5PDM379372 | rs40512 | 59775992 | 59775992 | 120628 | 120628 |
| SNP 25 | SNP5PDM380376 | | 59776995 | 59776995 | | |
| SNP 24 | SNP5PDM381086 | rs35385 | 59777706 | 59777706 | 118914 | 118914 |
| SNP 23 | SNP5PDM388220 | rs26953 | 59784839 | 59784839 | 111781 | 111781 |
| SNP 22 | SNP5PDM388748 | | 59785368 | 59785368 | 111252 | 111252 |
| SNP 21 | SNP5PDM388749 | rs26954 | 59785369 | 59785370 | | |
| SNP 20 | SNP5PDM390700 | | 59787319 | 59787319 | 109301 | 109301 |
| SNP 19 | SNP5PDM392152 | rs4133470 | 59788771 | 59788771 | 107849 | 107849 |
| SNP 18 | SNP5PDM392684 | | 59789302 | 59789302 | 107317 | 107317 |
| SNP 17 | SNP5PDM394085 | | 59790704 | 59790704 | 105792 | 105792 |
| SNP 16 | SNP5PDM394776 | rs35384 | 59791395 | 59791395 | 105225 | 105225 |
| SNP 15 | SNP5PDM395449 | rs35382 | 59792068 | 59792068 | 104552 | 104552 |
| SNP 14 | SNP5PDM397023 | rs26950 | 59793643 | 59793643 | 102977 | 102977 |
| SNP 13 | SNP5PDM399206 | rs26949 | 59795825 | 59795825 | 100795 | 100795 |
| SNP 12 | SNP5PDM400966 | rs153153 | 59797585 | 59797585 | 99035 | 99035 |
| SNP 11 | SNP5PDM402736 | rs152340 | 59799349 | 59799349 | | |
| SNP 10 | SNP5PDM407853 | | 59804468 | 59804468 | 92148 | 92148 |
| SNP 9 | SNP5PDM408531 | | 59805145 | 59805145 | 91470 | 91470 |
| SNP 8 | SNP5PDM408979 | | 59805593 | 59805593 | 91022 | 91022 |
| SNP 7 | SNP5PDM409460 | | 59806074 | 59806074 | 90541 | 90541 |
| SNP 6 | SNP5PDM411387 | | 59808001 | 59808001 | 88614 | 88614 |
| SNP 5 | SNP5PDM411544 | rs27564 | 59808159 | 59808159 | 88456 | 88456 |
| SNP 4 | SNP5PDM416882 | rs153152 | 59813496 | 59813496 | 83119 | 83119 |
| SNP 3 | SNP5PDM417756 | rs187481 | 59814371 | 59814371 | 82244 | 82244 |
| SNP 2 | SNP5PDM419874 | rs152341 | 59816488 | 59816488 | 80127 | 80127 |
| SNP 1 | SNP5PDM421449 | rs248911 | 59818063 | 59818063 | 78552 | 78552 |
| | D5S1990 | | 60945599 | 60945816 | | |
| | D5S1359 | | 63542603 | 63542894 | | |
| | D5S2089 | | 65914315 | 65914496 | | |
| | D5S647 | | 66217674 | 66218065 | | |
| | D5S2121 | | 66584091 | 66584385 | | |

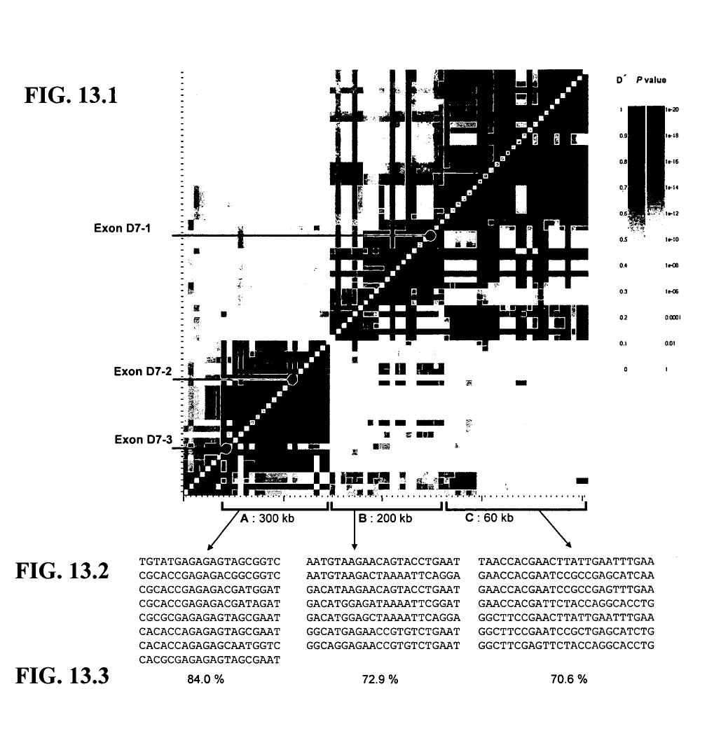

We next searched for SNPs in the intronic and flanking regions of PDE4D. The SNPs were identified in the public NCBI SNP database or by sequencing selected intronic and flanking regions in the gene in at least 94 patients and 94 controls. We initially identified 637 SNPS. Many of these SNPs were completely correlated so we removed many redundant SNPs from further genotyping. Some SNPs with very low minor allele frequencies were also ignored. This resulted in a set of 260 SNPs that were then genotyped for the entire patient and control cohorts. The preponderance of markers with significant associations was located at the 5′ end of the gene. One SNP (SNP5PDM76361; SNP83) for carotid stroke and five of the SNPs (SNP5PDM357221=SNP45, SNP5PDM361545=SNP41, SNP5PDM43741=SNP87, SNP5PDM29517=SNP89 and SNP5PDMSNP56) for the combined cardiogenic and carotid stroke remained significant even after adjusting for all the SNPs tested (Table 2D). Three of these significant SNPs flank exon D7-1; the other three are in a 100 kb region containing exon D7-2 (for physical positions see Table 2D). The two most significant SNPs, SNP45 and SNP41, are within 6 kb of the microsatellite marker AC008818-1, and the at-risk alleles of all three genetic markers are in strong linkage disequilibrium with D′>0.9 and p-value nearly zero. The square of the correlation (R²) is very high between the two SNPs (˜0.93), but is substantially lower (˜0.08) between each SNP and the at-risk allele of the microsatellite. This is due to the fact that the frequency of the at-risk alleles of the two SNPs are similar, and much more frequent than that for the at-risk allele of the microsatellite. The LD block structure around the 5′ end of PDE4D is displayed in FIG. 13.1. We delineate three blocks A, B and C encompassing the first three exons of PDE4D and its immediate downstream region. Exons D7-3 and D7-2 are both in block A, while D7-1 (the first exon) is in block B, but close to its border with block C. Given this block structure we were prepared to investigate the haplotype associated susceptibility to stroke in this region. Table 2D. All SNPs that show association with a p-value less than 0.01 for all stroke patients, all patients excluding hemorrhagic stroke and the combined cardiogenic and carotid stroke.

| Phenotype | Marker | Allele | p-value | RR | # Affect | Aff. % | # Ctrl | Ctrl. % |
|---|---|---|---|---|---|---|---|---|
| All patients | SNP 32 | C | 0.00024 | 1.46 | 400 | 37.9 | 475 | 29.5 |
| | SNP 56 | T | 0.0028 | 1.31 | 550 | 71.4 | 615 | 65.5 |
| | SNP 45 | G | 0.0065 | 1.33 | 723 | 82.4 | 492 | 78.0 |
| | SNP 48 | T | 0.0091 | 1.28 | 547 | 68.3 | 481 | 62.8 |
| All patients | SNP 32 | C | 0.00034 | 1.45 | 377 | 37.8 | 475 | 29.5 |
| excl. | SNP 56 | T | 0.0066 | 1.28 | 518 | 70.9 | 615 | 65.5 |
| hemorrhagic | SNP 45 | G | 0.0095 | 1.31 | 679 | 82.3 | 492 | 78.0 |
| stroke | | | | | | | | |
| Combined | SNP 45 | G | 0.000034* | 1.77 | 309 | 86.3 | 492 | 78.0 |
| cardiogenic & | SNP 41 | A | 0.000078* | 1.86 | 236 | 86.0 | 368 | 76.8 |
| carotid | SNP 87 | T | 0.00019* | 1.49 | 263 | 58.2 | 583 | 48.4 |
| | SNP 89 | A | 0.00025* | 1.84 | 232 | 88.8 | 450 | 81.1 |
| | SNP 56 | T | 0.00027* | 1.56 | 230 | 74.8 | 615 | 65.5 |
| | SNP 39 | T | 0.00032 | 1.58 | 326 | 84.4 | 589 | 77.3 |
| | SNP 91 | G | 0.00047 | 1.80 | 233 | 88.6 | 451 | 81.3 |
| | SNP 32 | C | 0.00069 | 1.61 | 144 | 40.3 | 475 | 29.5 |
| | SNP 62 | A | 0.00089 | 1.73 | 153 | 83.0 | 556 | 73.8 |
| | SNP 48 | T | 0.00080 | 1.51 | 229 | 71.8 | 481 | 62.8 |
| | SNP 42 | A | 0.0018 | 1.49 | 259 | 72.0 | 403 | 63.6 |
| | SNP 184 | G | 0.0025 | 1.68 | 252 | 90.7 | 570 | 85.3 |
| | SNP 58 | T | 0.0042 | 1.54 | 234 | 85.3 | 569 | 79.0 |
| | SNP 53 | C | 0.0041 | 1.58 | 146 | 36.0 | 269 | 26.2 |
| | SNP 97 | G | 0.0046 | 1.40 | 225 | 54.2 | 450 | 45.9 |
| | SNP 204 | A | 0.0049 | 1.32 | 334 | 63.9 | 651 | 57.3 |
| | SNP 8 | A | 0.0054 | 1.59 | 228 | 89.0 | 612 | 83.7 |
| | SNP 83 | C | 0.0074 | 1.39 | 223 | 60.1 | 349 | 52.0 |
| | SNP 43 | T | 0.0093 | 1.48 | 243 | 85.4 | 550 | 79.8 |

*significant after adjusting for multiple testing

Haplotype Analysis

Our general approach to haplotype analysis involves using likelihood-based inference applied to NEsted MOdels. The method is implemented in our program NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e, the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hⱼ are assumed to have the same risk, it corresponds to assuming that fᵢ/pᵢ=fⱼ/pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (e.g., see the information measure section below) and may require randomization to properly evaluate statistical significance. In NEMO, maximum likelihood estimates, likelihood ratios and p-values are calculated directly, with the aid of the EM algorithm, for the observed data treating it as a missing-data problem.

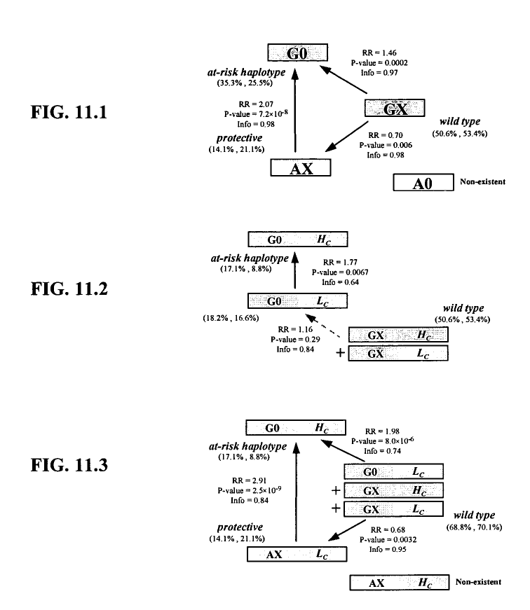

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes h₁=G0, h₂=GX and h₃=AX, and the focus is on comparing h₁ and h₂. The alternative grouping is [h₁], [h₂], [h₃] and the null grouping is [h₁, h₂], [h₃]. The actual problem we faced in FIG. 11.1 is actually slightly more complicated because allele X is a composite allele that includes five alleles other than allele 0, and hence GX and AX each correspond to five haplotypes. One could have collapsed these alleles into one at the data processing stage, and performed the test as described. This is a perfectly valid approach, and indeed, whether we collapse or not makes no difference if there were no missing information regarding phase. But, with the actual data, each of the 5 alleles making up X correlates differently with the SNP alleles and this provides some partial information on phase. Collapsing at the data processing stage will unnecessarily increase the amount of missing information. What was actually done is natural in the nested-models/partition framework. Let h₂ be split into h₂ₐ, h_{2b}, h₂ₑ, and h₃ be split into h₃ₐ, h_{3b}, . . . , h₃ₑ. Then the alternative grouping is [h₁], [h₂ₐ, h_{2b}, . . . , h₂ₑ], [h₃ₐ, h_{3b}, . . . , h₃ₑ] and the null grouping is [h₁, h₂ₐ, h_{2b}, . . . , h₂ₑ], [h₃ₐ, h_{3b}, . . . , h₃ₑ]. The same method is used to handle the composite haplotypes in FIG. 11.2 and 11.3 where collapsing at the data processing stage is not even an option since L_{C} represents multiple haplotypes constructed from 25 SNPs. Here, we also want to mention that, apart from the pair-wise comparisons presented in FIG. 11.1, a 3-way test with the alternative grouping of [h₁], [h₂ₐ, h_{2b}, . . . h₂ₑ], [h₃ₐ, h_{3b}, . . . , h₃ₑ] versus the null grouping of [h₁, h₂ₐ, h_{2b}, . . . , h₂ₑ, h₃ₐ, h_{3b}, . . . , h₃ₑ] could also be performed. Note that the generalized likelihood ratio test-statistic would have two degrees of freedom instead of one. We actually have performed this test and it gave a p-value of 2.4×10⁻⁷.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose we calculate the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose we then perform a likelihood ratio test treating these estimated haplotype counts as though they are the actual counts. We could also perform a Fisher's exact test, but we would then need to round off these estimated counts since they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than Λ, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

Haplotype Association

We first considered haplotypes based on the most significantly associated SNPs and microsatellite, SNP45, SNP41 and AC008818-1, which are all in block B and are separated by only 6 kb. Not surprisingly given the high degree of correlation between SNP45 and SNP41, we found that it was sufficient to consider only the two marker haplotypes consisting of the microsatellite and SNP45—the SNP with the higher genotype yield. The results of this association study for the combination of carotid and cardiogenic stroke are displayed in FIG. 11.1. Note that, for convenience, we have designated by the letter X the joint set of alleles that are not the at-risk allele, 0, of microsatellite AC008818-1. Thus, GX should be understood as the composite of all haplotypes including the G nucleotide of SNP45 except for the G0 haplotype. For our samples, the AO haplotype does not exist. This suggests that allele 0 originated in a haplotype background with allele G of SNP45, and since then no recombination has occurred between those two markers for chromosomes that carried allele 0. AX, G0 and GX have significantly distinct risks for the combined carotid and cardiogenic stroke phenotype. We refer to GX as the wild type because it is the most common (53.4% in controls) and also because it has the intermediate level risk that is not too different from the population risk. The haplotype G0 has increased risk and AX is protective, with risks of 1.46 and 0.70 relative to the wild type, respectively. The G0 risk is 2.07 times that of the protective haplotype AX. Each of the three pairwise comparisons is highly significant, with p-values ranging from 0.006 to 7.2×10⁻⁸. It is interesting to observe that even though both AX and GX are composite haplotypes, the AX haplotype can be simply summarized by the allele A of SNP45, since the A0 haplotype does not exist. For a similar reason, the G0 haplotype is completely determined by the 0 allele of AC008818-1. Also displayed in FIG. 11.1 is the information content (Info) of each test. The difference between Info and 1 is a measure of the information that is lost due to the uncertainty with phase and missing genotypes. Note that Info is very close to 1 for each of the three tests in FIG. 11.1. That is a result of SNP45 and AC008818-1 being in very strong LD. Note that tests presented later in FIG. 11.2 and 11.3, involving longer haplotypes have lower information content.

We next identified and estimated the risks for the common SNP haplotypes within each block. For this portion of the analysis only those SNPs with minor allele frequency greater than 20% were considered. Block A (300 kb) contained 19 such SNPs, block B (200 kb) 22 SNPs, and block C (60 kb) 25 SNPs. All haplotypes within each block with an estimated frequency in the population of 2% or greater have been identified. Within each block there were fewer than ten such haplotypes, and they accounted for approximately 80% of the total haplotype frequency for that block. A brief schematic of the identified haplotypes are displayed in FIG. 13.2 and the risks and frequencies of these haplotypes are available in Table 3. Within block A no common haplotype has greater risk than SNP87 alone. The strongest signals were for haplotypes in block B and C. Each block contained a haplotype significantly associated with the combination of carotid and cardiogenic stroke and having relative risk around 1.5. The common at-risk haplotype in block B is the SNP background of the G0 haplotype previously identified.

While there were no significant single marker associations in block C, a common haplotype with 15.4% frequency in controls was observed. We designate this haplotype H_{C}. Investigation of the contribution of H_{C} in conjunction with the SNP45 and AC008818-1 haplotypes leads to another interesting observation. For notation, all haplotypes defined by the 25 SNPs in block C that are not H_{C} are jointly denoted by the composite haplotype L_{C}. First, it is noted that AX and H_{C} do not exist together on the same chromosome (see FIG. 11.3), at least in these samples, and thus blocks B and C are far from being independent. As a consequence, the extended composite haplotype AXL_{C} is the same as AX. The haplotype G0 can be split into the two extended haplotypes G0H_{C} and the composite G0L_{C}, which, as indicated in FIG. 11.2, have significantly different risks (p value=0.0067). Moreover, it appears that the elevated risk of G0 is totally accounted for by G0H_{C} as G0L_{C} has risk that is not significantly different from GX=GXH_{C}+GXL_{C} (see FIG. 11.2). This observation allows us to refine the haplotype groupings of FIG. 13.1 into the groupings indicated by FIG. 13.3. The extended at-risk haplotype G0H_{C} (8.8% in controls) and protective composite haplotype AXL_{C} (21.1% in controls), have, respectively, relative risks of 1.98 and 0.68 compared to the wild type (70.1% in controls). Based on these risk estimates, if everybody's risk can be made to correspond to that of a homozygote carrier of the protective variant, the number of cases would be reduced by 55%, which can be interpreted as the population attributed risk of the at-risk haplotype and the wild type combined.

The at-risk haplotype G0H_{C} spans a region of about 64 kb. While it is possible that the increased risk is due to multiple polymorphisms over that region, the results are also consistent with a relatively recent mutation, as yet to be identified, which occurred in that haplotype background, and since then no recombination has occurred in that extended region for chromosomes carrying the mutation. By contrast, the protective composite haplotype AXL_{C} can be simply represented by allele A of SNP45. Hence, it is possible that allele A of SNP45 is the functional protective variant, although it is possible that the functional variant is simply in strong LD with allele A of SNP45 and has yet to be identified. Indeed, statistically, the effects of SNP45 and SNP41 are indistinguishable from each other.

Statistical Analysis.

For single marker association to the disease, the Fisher exact test was used to calculate two-sided p-values for each individual allele. All p-values were presented unadjusted for multiple comparisons unless specifically indicated. The presented frequencies (for microsatellites, SNPs and haplotypes) were allelic frequencies as opposed to carrier frequencies. To minimize any bias due the relatedness of the patients who were recruited as families for the linkage analysis, we eliminated first and second-degree relatives from the patient list. Furthermore, we have repeated the test for association correcting for any remaining relatedness among the patients, by extending a variance adjustment procedure described in Risch, N. & Teng, J. (Genome Res., 8:1278-1288 (1998)). The relative power of family-based and case-control designs for linkage disequilibrium studies of complex human diseases I. DNA pooling. (ibid) for sibships so that it can be applied to general familial relationships, and present both adjusted and unadjusted p-values for comparison. The differences are in general very small as expected. To assess the significance of single-marker association corrected for multiple testing we carried out a randomisation test using the same genotype data. We randomised the cohorts of patients and controls and redid the association analysis. This procedure was repeated up to 500,000 times and the p-value we presented is the fraction of replications that produced a p-value for some marker allele that is lower than or equal to the p-value we observed using the original patient and control cohorts.

For both single-marker and haplotype analyses, relative risk (RR) and the population attributable risk (PAR) were calculated assuming a multiplicative model (haplotype relative risk model), (Terwilliger, J. D. & Ott, J., Hum Hered, 42, 337-46 (1992) and Falk, C. T. & Rubinstein, P, Ann Hum Genet 51 (Pt 3), 227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes hᵢ and hⱼ, risk(hᵢ)/risk(hⱼ)=(fᵢ/pᵢ)/(fⱼ/pⱼ), where f and p denote respectively frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program called NEMO, which stands for NEsted MOdels, was used to calculate all the haplotype results presented. To handle uncertainties with phase and missing genotypes, it is emphasized that we do not use a common two-step approach to association tests, where haplotype counts are first estimated, possibly with the use of the EM algorithm, Dempster, (A. P., Laird, N. M. & Rubin, D. B., Journal of the Royal Statistical Society B, 39, 1-38 (1971)) and then tests are performed treating the estimated counts as though they are true counts, a method that can sometimes be problematic and may require randomisation to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests we performed, with results presented in Table 3, compare one selected haplotype against all the others. Call the selected haplotype h₁ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=p₁ for all i. The alternative model we use for the test assumes h₂, . . . , hₖ to have the same risk while h₁ is allowed to have a different risk. This implies that while p₁ can be different from f₁, f₁/(f₂+ . . . +fₖ)=pᵢ/(p₂+ . . . +pₖ)=βᵢ for i=2, . . . , k. Denoting f₁/p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)]
where l denotes logₑlikelihood and ~ and ˆ denote maximum likelihood estimates under the null hypothesis and alternative hypothesis respectively. Λ has asymptotically a chi-square distribution with 1-df, under the null hypothesis and it was used to compute p-values presented in Table 3. The tests presented in FIG. 11 have slightly more complicated null and alternative hypotheses. For the results in FIG. 11, let h₁ be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows all three haplotype groups to have different risks. This implies that, under the null hypothesis, there is a constraint that f₁/p₁=f₂/p₂, or w=[f₁/p₁]/[f₂/p_{2]=1}. The test statistic based on generalized likelihood ratios is
Λ=2[l(\hat{p}₁, \hat{f}₁, \hat{p}₂, ŵ)−l(\tilde{p}₁, \tilde{f}₁, \tilde{p}₂, 1)]
that again has asymptotically a chi-square distribution with 1-df under the null hypothesis. There is actually an extra complication to the test due to h₂ and h₃ being composite haplotypes. That is handled in a natural manner under the nested models framework. Other tests presented in FIG. 11.2 and 11.3 were similarly performed.

LD between pairs of SNPs was calculated using the standard definition of D′ and R² (Lewontin, R., Genetics 49, 49-67 (1964) and Hill, W. G. & Robertson, A. Theor. Appl. Genet. 22,226-231 (1968)). Using NEMO, frequencies of the two marker allele combinations are estimated by maximum likelihood and deviation from linkage equilibrium is evaluated by a likelihood ratio test. The definitions of D′ and R² were extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots we present the markers are plotted equidistant rather than according to their physical location.

TABLE 3
Haplotype diversity at the 5′end of the PDE4D gene.
All haplotypes shown that have >2% population frequency within each of the 3 blocks
of strong LD together with the haplotype association results comparing the combination
of cardiogenic and carotid stroke versus controls.
Block A:

| SNP 102 | SNP 101 | SNP 100 | SNP 99 | SNP 98 | SNP 97 | SNP 96 | SNP 95 | SNP 93 | SNP 92 | SNP 90 | SNP 88 |
|---|---|---|---|---|---|---|---|---|---|---|---|
| T | G | T | A | T | G | A | G | A | G | A | G |
| C | G | C | A | C | C | G | A | G | A | G | A |
| C | G | C | A | C | C | G | A | G | A | G | A |
| C | G | C | A | C | C | G | A | G | A | G | A |
| C | G | C | G | C | G | A | G | A | G | A | G |
| C | A | C | A | C | C | A | G | A | G | A | G |
| C | A | C | A | C | C | A | G | A | G | A | G |
| C | A | C | G | C | G | A | G | A | G | A | G |

| SNP 87 | SNP 86 | SNP 84 | SNP 83 | SNP 82 | SNP 81 | SNP 80 | SNP 79 | p-value | Aff % | Ctrl % | RR |
|---|---|---|---|---|---|---|---|---|---|---|---|
| T | A | G | C | G | G | T | C | 0.0302 | 26.7 | 22.2 | 1.28 |
| C | G | G | C | G | G | T | C | 0.366 | 2.1 | 2.9 | 0.73 |
| C | G | A | T | G | G | A | T | 0.303 | 2.6 | 3.6 | 0.71 |
| C | G | A | T | A | G | A | T | 0.335 | 22.5 | 24.5 | 0.89 |
| T | A | G | C | G | A | A | T | 0.216 | 12.6 | 10.6 | 1.23 |
| T | A | G | C | G | A | A | T | 0.876 | 2.2 | 2.4 | 0.94 |
| C | A | A | T | G | G | T | C | 0.001 | 6.5 | 11.2 | 0.55 |
| T | A | G | C | G | A | A | T | 0.401 | 7.9 | 6.6 | 1.20 |

Block B:

| SNP-77 | SNP-76 | SNP-73 | SNP-71 | SNP-69 | SNP-67 | SNP-66 | SNP-64 | SNP-63 | SNP-62 | SNP-61 | SNP-59 | SNP-57 |
|---|---|---|---|---|---|---|---|---|---|---|---|---|
| A | A | T | G | T | A | A | G | A | A | C | A | G |
| A | A | T | G | T | A | A | G | A | C | T | A | A |
| G | A | C | A | T | A | A | G | A | A | C | A | G |
| G | A | C | A | T | G | G | A | G | A | T | A | A |
| G | A | C | A | T | G | G | A | G | C | T | A | A |
| G | G | C | A | T | G | A | G | A | A | C | C | G |

| SNP-56 | SNP-54 | SNP-53 | SNP-49 | SNP-48 | SNP-45 | SNP-42 | SNP-41 | SNP-39 | p-value | Aff % | Ctrl % | RR |
|---|---|---|---|---|---|---|---|---|---|---|---|---|
| T | A | C | C | T | G | A | A | T | 0.0004 | 29.2 | 21.4 | 1.52 |
| A | A | T | T | C | A | G | G | A | 0.007 | 4.3 | 7.6 | 0.55 |
| T | A | C | C | T | G | A | A | T | 0.958 | 2.0 | 2.1 | 0.98 |
| A | A | T | T | C | G | G | A | T | 0.610 | 6.2 | 5.6 | 1.13 |
| A | A | T | T | C | A | G | G | A | 0.0104 | 3.4 | 6.3 | 0.52 |
| T | G | T | C | T | G | A | A | T | 0.803 | 14.6 | 14.1 | 1.04 |

Block C:

| SNP 37 | SNP 35 | SNP 34 | SNP 32 | SNP 31 | SNP 30 | SNP 28 | SNP 27 | SNP 26 | SNP 24 | SNP 23 | SNP 22 | SNP 20 | SNP 19 |
|---|---|---|---|---|---|---|---|---|---|---|---|---|---|
| T | A | A | C | C | A | C | G | A | A | C | T | T | A |
| G | A | A | C | C | A | C | G | A | A | T | C | C | G |
| G | A | A | C | C | A | C | G | A | A | T | C | C | G |
| G | A | A | C | C | A | C | G | A | T | T | C | T | A |
| G | G | C | T | T | C | C | G | A | A | C | T | T | A |
| G | G | C | T | T | C | C | G | A | A | T | C | C | G |
| G | G | C | T | T | C | G | A | G | T | T | C | T | A |

| | | | | | | | | | | | | Aff | Ctrl | |
|---|---|---|---|---|---|---|---|---|---|---|---|---|---|---|
| SNP 16 | SNP 15 | SNP 14 | SNP 13 | SNP 12 | SNP 6 | SNP 5 | SNP 4 | SNP 3 | SNP 2 | SNP 1 | p-value | % | % | RR |
| T | T | G | A | A | T | T | T | G | A | A | 0.0006 | 22.2 | 15.4 | 1.58 |
| C | C | G | A | G | C | A | T | C | A | A | 0.533 | 2.2 | 2.8 | 0.78 |
| C | C | G | A | G | T | T | T | G | A | A | 0.24 | 3.0 | 2.0 | 1.52 |
| C | C | A | G | G | C | A | C | C | T | G | 0.302 | 2.6 | 1.8 | 1.46 |
| T | T | G | A | A | T | T | T | G | A | A | 0.258 | 2.0 | 3.0 | 0.68 |
| C | T | G | A | G | C | A | T | C | T | G | 0.0078 | 7.8 | 12.0 | 0.62 |
| C | C | A | G | G | C | A | C | C | T | G | 0.781 | 32.9 | 33.6 | 0.97 |

Allelic definitions and polymorphisms for SNPs in the two most significant haplotypes (in block B and C).

The analysis presented above represents a conservative analysis of the data since it restricted the analysis to SNPs with minor allelic frequencies of greater than 20%. To further understand the magnitude of the contribution of PDE4D to stroke in this 5 prime region, we repeated the analysis without such restrictions, including all SNPs selected for genotyping. We found a SNP haplotype for the two major subtypes of ischemic stroke, carotid and cardiogenic stroke (Table 3, Block C). This is a 5 SNP haplotype that covers an area of 48 kb and is just upstream of the 5′exon covering the presumed promoter region of isoform PDE4D7. It captures the same information as the 0 allele for marker AC008818-1. However, the SNP haplotype is more specific in the sense that it has a higher relative risk, i.e., 2.3. This haplotype is carried by 47% of the patients and has the same population attributable risk (PAR) of 0.25. The polymorphisms and alleles for the SNPs are presented in Table 4A.

TABLE 4A

| | Public | | | |
|---|---|---|---|---|
| SNP | name if | | | Allele |
| Name | available | Polymorphism | Position | (nucleotide) |
| SNP42 | rs153031 | A/G | 138806 | 0 (A) |
| SNP5PDM361194 | | | | |
| SNP34 | rs27653 | C/A | 131865 | 0 (A) |
| SNP5PDM368135 | | | | |
| SNP32 | rs456009 | C/T | 129361 | 1 (C) |
| SNP5PDM370640 | | | | |
| SNP26 | rs40512 | G/A | 120628 | 0 (A) |
| SNP5PDM379372 | | | | |
| SNP9 | (new) | G/A | 91470 | 0 (A) |
| SNP5PDM408531 | | | | |

TABLE 4B

| | | | | | | | # | Aff. | # | Ctrl. | | | |
|---|---|---|---|---|---|---|---|---|---|---|---|---|---|
| Phenotype | SNP42 | SNP34 | SNP32 | SNP26 | SNP9 | p-value | Affect | Freq.* | Ctrl | Freq* | R-risk | PAR | info |
| All stroke | A | A | C | A | A | 2.17E−05 | 988 | 0.19 | 652 | 0.12 | 1.8 | 0.16 | 0.604 |
| Cardiogenic/ | A | A | C | A | A | 3.37E−07 | 313 | 0.236 | 652 | 0.119 | 2.3 | 0.25 | 0.616 |
| carotid | | | | | | | | | | | | | |

allelic frequency

The sequences for the microsatellite markers are as follows:

AC008879-2 amplimer:

(SEQ ID NO: 85)

ACAAAGAGCACCTTTCCAGTGGACAACTAACTAAAGTGGTGTGATTTTGG
TATAAGTTTGTGTGTGTGTGTGTGTGTGTGTTGTGTGTGTGTGTATGTGT
ATACATTTAGTTTTATTGTAACAAAGCAACTTGTACTTTTCACGTTTAAA
A

AC008879-2, allele 0 is the same allele as the minimum allele observed in CEPH 1347-02, family 137, individual 02.

In summary, this single SNP haplotype (which is only one haplotype of the several found above but is probably the most tightly associated to stroke) more than doubles an individual's risk for cardiogenic and carotid stroke and accounts for 25% of such strokes in Iceland. The other haplotypes described above provide additional risk for stroke. The magnitude of this risk haplotype is comparable or higher than the well-known clinical risk factors for stroke such as hypertension, diabetes, hyperlipidemia, and smoking.

These SNPs show strong association in patients with cardioembolic and large vessel disease.

Table 5 and Table 6 show previously known microsatellite markers and novel microsatellites in sequence. Forward and reverse primers are shown.

TABLE 5
Previously Known microsatellite markers in sequence

| | | | | |
|---|---|---|---|---|
| | | SEQ ID | | SEQ ID |
| Accession number | Forward primer | NO. | Reverse primer | NO. |

| | | | | | |
|---|---|---|---|---|---|
| D5S2107 | GDB:614475 | GCCTTTGGGCCAACA | 15 | CAAACCAACAGGAGTATGTACTTTT | 16 |
| D5S468 | GDB:593646 | AATGAATGGTAGATTLTAACCTGAG | 17 | TGGGAAAATAAATACATGCG | 18 |
| D5S2000 | GDB:608769 | TTATACCAGGAGAGTAGACTTTTTT | 19 | CATGGTAATTITCAAATATGAGAG | 20 |
| D5S2091 | GDB:613806 | GCATTTGTCATGTGCCA | 21 | GGTATTITCATTCACAGCCAGTC | 22 |
| D5S2500 | GDB:683034 | TTAAAGGAGTGATCTCCCCC | 23 | GTTACAGTACCTATGGTCATGCG | 24 |
| D5S2080 | GDB:613188 | GCACTGTGAATTTCAAATG | 25 | GTCAGGGGACTGGGAT | 26 |
| D5S2018 | GDB:609957 | CCTGTAAACAATGAAAACCCACTGA | 27 | GACTATGCTGTGTGTGTGGCTG | 28 |
| D5S2071 | GDB:612756 | TCTGGGTTTACAACCTTCAAA | 29 | TAACTGGCTTGGCCCG | 30 |

TABLE 6
Novel microsatellites in sequence:

| | | | |
|---|---|---|---|
| Forward primer | SEQ ID NO. | Reverse primer | SEQ ID NO. |

| | | | | |
|---|---|---|---|---|
| DG5S382 | CAGTAAATAGTTTGCTTCAGGCATT | 31 | CTCATACTCTGCGTGGCTTG | 32 |
| AC008829-5 | AGGGCTAAGTGGATCACAGC | 33 | AGAGGGTCTTGCCACTGTGT | 34 |
| AC008833-2 | TCTGCAAGACTCTCGGTGCT | 35 | TGCAGATCTCATATTTCCATGTTT | 36 |
| AC008833-3 | TCTGCCCTTTGTTCCTCATC | 37 | GTCAAGGGAGTGATGGCAGT | 38 |
| AC022125-3 | AAAATGACTGGCTCCCACAA | 39 | GGGAAATCATACTGCCCTCA | 40 |
| AC008833-6 | AAACATAGCCACCCTGTTGC | 41 | TCCAAAGCCCTTAGCTTAATCA | 42 |
| D17-C | GCTCCCTGGACTGTGGTAAA | 43 | GCCACATTGCTGTCACATTT | 44 |
| D17-B | TTTTTCAGGGCTGGGTAGAA | 45 | TCCAAAGGAAGTGAAATCAGTG | 46 |
| D17-D | CTAACCCATCCTCACCCAAT | 47 | TGTGGCATACAGGGAAGTGA | 48 |
| AC008804-1 | GTGCTGGAATTTGGCTCCTA | 49 | CAAACATCATTTTGCCTTGC | 50 |
| AC008804-2 | TCCCAAACGATAGCTGTTGC | 51 | GAATTAGGACGGTGGCTCAA | 52 |
| AC008804-3 | TTTGCATTCATCACTCATTCG | 53 | CCCGTAGCATCTGATCCAGT | 54 |
| D17-H | AGAAAGCTTCCCCTCCACTG | 55 | CATTCCAGCCTGAGCTACAA | 56 |
| D17-G | TGGGCTCCAATTATCCTTCC | 57 | TGCAGTTTGCACTCTCCTTG | 58 |
| AC027322-12 | TTATCTGTTCCCCATGCTTTT | 59 | TGTTACATCTFGATCTATGACGTTT | 60 |
| AC027322-10 | TGTATCCTGCATCCCTTGTT | 61 | GGAATAACCCAAAAGTAATTGTAGTGA | 62 |
| AC027322-9 | TCGTGCCAAGATGAAAATGA | 63 | AAACCTCCCTGATCATCTGAA | 64 |
| AC027322-8 | ACAGAGGAGCAAAGGAATCA | 65 | TTGGCACGAATCACTCTCTG | 66 |
| AC027322-3 | CCCCATTTGGATGATGGTAA | 67 | TGAGAACATCTAACGTCTTTTTCAA | 68 |
| AC027322-5 | GGCACAGATAACTGGGAAGC | 69 | CCCCCAAAAGTACTGCATAAA | 70 |
| DG5S397 | ATGTTGGCATTTGGTGAGGT | 71 | CACCTGTGCCTTTCCAGTA | 72 |
| AC008879-2 | TAAACGTGAAAAGTACAAGTTGC | 73 | ACAAAGAGCACCTTTCCAGTG | 74 |
| *AC008818-1 | TGCTTGGTGAAGGAATAGCC | 75 | GAGCCTGGGTFCTCAGGAAT | 76 |
| **AC008879-3 | GGCAAGAACAGTTTGGAGGA | 77 | GACTGCTGTTTGCTGGTTGA | 78 |
| AC020733-1 | AAATGGCTATAAAGTGCTTTGAAC | 79 | CGGTCTCAACAACCAGAACA | 80 |
| AC016591-2 | CAGAAACACACAGAAGTCATTCAA | 81 | CAGACCCAATTAATGGCAAAA | 82 |
| DG5S405 | TCTGTCTTCTTTGACCCATGAAT | 83 | CAACACAGCGAGACCTCATC | 84 |

*Product Size 194, tetranucleotide repeat
**Product Size 150; dinucleotide repeat

TABLE 7
Correlation between at-risk alleles for markers AC008818-1,
SNP45 and SNP41. Estimates of LD (correlation) between
the at-risk alleles, allele 0 for marker AC008818-1, allele G for
SNP45 and allele A for SNP41, the three most significant
disease associated genetic markers.

| | R² | | |
|---|---|---|---|
| Frequency | AC008818-1 | SNP 45 | SNP 41 |

A. Combined cardiogenic and carotid patients

| | | | | | |
|---|---|---|---|---|---|
| D′ | AC008818-1 | 0.355 | | 0.090 | 0.076 |
| | SNP 45 | 0.863 | 1 | | 0.943 |
| | SNP 41 | 0.860 | 0.906 | 0.981 | |

B. Controls

| | | | | | |
|---|---|---|---|---|---|
| D′ | AC008818-1 | 0.255 | | 0.091 | 0.078 |
| | SNP 45 | 0.780 | 1 | | 0.920 |
| | SNP 41 | 0.768 | 0.924 | 0.968 | |

TABLE 8
Association of risk factors.
Association of microsatellite AC008818-1 at-risk allele 0, SNP45 allele A and haplotype
G0H_{c} respectively with various risk factors.
Cases are stroke patients with risk factors and controls are stroke patients without the
risk factors. P-values are two-sided.

| | AC008818-1:Allele 0 | | | | | SNP45:Allele A | | | | | Haplotype G0H_{c} | | | | |
|---|---|---|---|---|---|---|---|---|---|---|---|---|---|---|---|
| | | | Cases | | | | | Cases | | | | | Cases | | |
| | Cases with | | without risk | | | Cases with | | without | | | Cases with | | without | | |
| | risk factor | | factor | | P- | risk factor | | risk factor | | | risk factor | | risk factor | | P- |
| | N | Frq. | N | Frq. | value | N | Frq. | N | Frq. | P-value | N | Frq. | N | Frq. | value |
| Hypertension | 477 | 0.303 | 203 | 0.303 | 1.000 | 416 | 0.172 | 181 | 0.188 | 0.510 | 503 | 0.134 | 216 | 0.123 | 0.634 |
| Hyper- | 274 | 0.336 | 312 | 0.271 | 0.025 | 242 | 0.216 | 277 | 0.186 | 0.516 | 287 | 0.153 | 329 | 0.104 | 0.026 |
| cholesterolemi | | | | | | | | | | | | | | | |
| Diabetes | 93 | 0.274 | 424 | 0.310 | 0.379 | 79 | 0.196 | 398 | 0.176 | 0.422 | 100 | 0.127 | 455 | 0.133 | 0.857 |
| Peripheral | 133 | 0.297 | 357 | 0.305 | 0.815 | 116 | 0.181 | 340 | 0.176 | 0.921 | 138 | 0.121 | 388 | 0.132 | 0.697 |
| artery | | | | | | | | | | | | | | | |
| occlusive | | | | | | | | | | | | | | | |
| Coronary | 179 | 0.302 | 429 | 0.318 | 0.588 | 153 | 0.170 | 406 | 0.182 | 0.662 | 181 | 0.122 | 467 | 0.141 | 0.444 |
| artery disease | | | | | | | | | | | | | | | |
| Early onset | 349 | 0.294 | 462 | 0.304 | 0.137 | 314 | 0.186 | 430 | 0.173 | 0.538 | 380 | 0.128 | 506 | 0.125 | 0.876 |
| (<68) | | | | | | | | | | | | | | | |
| Males vs | 457 | 0.291 | 358 | 0.310 | 0.414 | 420 | 0.181 | 303 | 0.168 | 0.575 | 489 | 0.122 | 370 | 0.141 | 0.315 |
| females | | | | | | | | | | | | | | | |

Discussion of Stroke Gene Identification

Genealogy, a comprehensive population-based list of broadly defined stroke patients and non-parametric allele sharing methods have been combined to successfully map a major gene to chromosome 5 for one of the most complex diseases known. We then used a large case-control association study that showed that PDE4D is the gene in this location that is the gene conferring substantial risk for stroke. This is the first gene ever mapped and isolated for the common forms of stroke. There was no correlation between the contribution of the families to this gene location and hypertension, diabetes or hyperlipidemias and this gene does not match any known gene contributing to these risk factors. The types of stroke studied in this work do not reflect a rare or Icelandic-specific form of stroke; rather, the diversity of the stroke phenotypes in Icelanders as well as risk factors are similar to those of most other Caucasian populations (Agnarsson, U., et al., Ann. Intern. Med., 130:987 (1999); Eliasson, J. H., et al., Loeknabladid, 85:517-25 (1999); Sveinbjörnsdottir, S., et al., Systematic registration of patients with Stroke and TIA admitted to The National University Hospital, Reykjavik, Iceland, in 1997, XIII. Meeting of the Icelandic Association in Internal Medicine, Akureyri, Iceland (Valdimarsson, E. M., et al., Loeknabladid 84:921 (1998)).

The magnitude of the risk and the frequency of the disease haplotypes in the general population confirm that we have mapped a gene for the common forms of stroke and not some rare form of stroke. This gene almost doubles one's risk for stroke in general, and more than doubles one's risk for the two most common subtypes of stroke, carotid and cardiogenic stroke. In addition, the most common disease haplotype has a population attributed risk of 25% (which means it accounts for 25% of the patients) and there are other haplotypes that we describe herein that are less common that accounts for other patients. Thus PDE4D is a major cause of stroke and its relative risk rivals those of hypertension, smoking, diabetes, and hyperlipidemia. PDE4D shows tighter correlation to the forms of stroke dependent on atherosclerosis (carotid and cardiogenic stroke) and it is expressed in cell types known to be important for atherosclerosis such as vascular smooth muscle cells, macrophages, and endothelial cells. This suggests that the strong effect that PDE4D variation has on stroke risk is through its role in the vascular biology of atherosclerosis (see discussion at the end of the examples). Example 2 details our sequencing of the entire PDE4D gene and the definition of its exon-intron structure based on new and old cDNAs, and Example 3 shows that the expression pattern of PDE4D isoforms correlates with a stroke associated haplotype.

### Example 2

### Sequencing and Characterization of the Human Gene and its RNA/Protein Isoforms

Sequence of the Stroke Gene Region

At the start of our work, there was little genomic sequence available in the public domain covering the stroke gene region. Therefore, we sequenced approximately 3 Mb of the area defined by one drop in lod. The locus on 5q12 indicated in the genome wide scan was physically mapped using bacterial artificial chromosomes (BACs). A set of overlapping clones for a 20 cM region was assembled through a combination of hybridization and BAC-fingerprint walking. Eighteen BACs (bacterial artificial clones) (RP11-164A5, RP11-188115, RP11-313P15, RP11-631M6, RP11-103A15, RP11-489L13, RP11-621C19, RP11-113C1, RP11-567M18, RP11-412M9, RP11-15G2, RP11-15F7, RP11-281M3, RP11-421L6, RP11-1A7, RP11-68E13, RP11-379P8, and RP11-422K3) covering the minimum tiling path of the one LOD interval were analysed using shotgun cloning and sequencing. Dye terminator (ABI PRISM BigDye) chemistry was used for fluorescent automated DNA sequencing. ABI prism 377 sequences were used to collect data and the Phred/Phrap/Consed software package in combination with the Polyphred software were used to assemble sequences (See Table 9A and 9B) Publicly available sequences (AC008836, AC073546, AC021603, AC008498, AC016435, AC021601, AC016591, AC008818, AC008879, AC008934, AC01929, AC027322, AC008111, AC020924, AC026693, AC012315, AC08804, AC008791, AC020975, AC008833, AC008829, AC022125, AC008790, AC026095, AC066693, AC008852, AC016642, AC034250, AC025179, AC08814, AC008926, AC010391, AC016635 and AC016604) from this region were assembled with the obtained sequence and a 3.7 Mb sequence (with 22 gaps) was generated. Comparison of the current public human assembly (NCBI BUILD 33) to our sequence of the STRK1 locus only showed a minor discrepance.

The BAC clones we sequenced are from the RCPI-11 Human BAC library (Pieter dejong, Roswell Park). The vector used was pBACe3.6. The clones were picked into a 94 well microtiter plate containing LB/chloramphenicol (25 μg/ml)/glycerol (7.5%) and stored at −80° C. after a single colony has been positively identified through sequencing. The clones can then be streaked out on a LB agar plate with the appropriate antibiotic, chloramphenicol (25 μg/ml)/sucrose (5%).

TABLE 9A

| Sequenced at Decode | | |
|---|---|---|
| (BAC name) | Comment | Accession number |
| RP11-621C19 | 1 | AC020733 |
| RP11-113C1 | 2 | |
| RP11-412M9 | 2 | |
| RP11-151G2 | 2 | |
| RP11-151F7 | 2 | |
| RP11-281M3 | 2 | |
| RP11-421L6 | 2 | |
| RP11-68E13 | 2 | |
| RP11-379P8 | 2 | |
| RP11-1A7 | 1 | AC008111 |
| RP11-422K3 | 2 | |

Key to “Comment” column:
1 = This BAC has a publicly available sequence, it was sequenced at Decode to make sure the sequence was correct
2 = Only BAC end-sequence available for this BAC publicly.

TABLE 9B

| Sequences available from | | |
|---|---|---|
| GenBank (BAC name) | Accession number | Status of sequence |
| RP11-621C19 | AC020733 | 17 unordered pieces |
| CTD-2003D5 | AC016591 | complete sequence |
| CTD-2210C1 | AC008879 | 7 unordered pieces |
| CTD-2124H11 | AC008818 | complete sequence |
| CTD-2301A11 | AC008934 | complete sequence |
| RP11-16B11 | AC011929 | 7 unordered pieces |
| CTC-261E10 | AC026693 | complete sequence |
| CTD-2027G10 | AC027322 | complete sequence |
| RP11-1A7 | AC008111 | 8 unordered pieces |
| CTD-2122K7 | AC012315 | complete sequence |
| CTD-2085F10 | AC008804 | complete sequence |
| CTD-2040J22 | AC008791 | complete sequence |
| RP11-235N16 | AC020975 | 16 ordered pieces |
| CTD-2146O16 | AC008833 | complete sequence |
| CTD-2084I4 | AC022125 | 17 ordered pieces |
| CTD-2140K22 | AC008829 | 26 ordered pieces |
| CTD-2124D11 | AC020924 | 7 ordered pieces |
| RP11-731H6 | AC026095 | 21 unordered pieces |

PDE4D Gene; Identification of New Exons and Splice Variants

The gene, human cAMP specific phosphodiesterase 4D (HPDE4D) was identified in the sequenced region by BLAST of our novel genomic sequence with the cDNAs/EST databases from GenBank. In addition, we ran RT-PCR reactions and 5 prime and 3 prime RACE reactions using cDNA libraries generated from a variety of tissues including human aorta. The primer sites used corresponded to known or exons predicted from our genomic sequence using Genscan, and Fgene. We found several novel cDNAs and matched them to the 3 Mb sequence in and around PDE4D. The genomic sequence covering all known and novel exons in PDE4D so far is approximately 1,550,000 bases in length.

We defined new alternative transcripts which together with previously known transcripts showed that the PDE4D gene contains 22 exons over at least 1.5 Mb and overlaps with the PART1 gene whose transcript is on the other strand at the 5′ end. The PDE4D gene has at least 7 promoters and encodes 8 protein isoforms. All isoforms have an identical C-terminal catalytic domain but differ at the N-terminal regulatory domain. Six of the 8 forms are so called long isoforms. Each of them have unique N-terminal regulatory domains but they are all characterized by two highly conserved regions found in all PDE4 subfamilies, i.e. upstream conserved regions 1 and 2 (UCR 1 and 2). The six long forms differ from each other by unique alternative 5 prime exons which predicts six alternative promoters that are each upstream of the corresponding 5 prime exon. The remaining two are the so-called short forms, variants that lack the UCR 1 (Houslay, M. D. & Adams, D. R., Biochem J, 370, 1-18 (2003)). The five previously known isoforms are encoded by 17 exons distributed over a segment of 0.9 Mb.

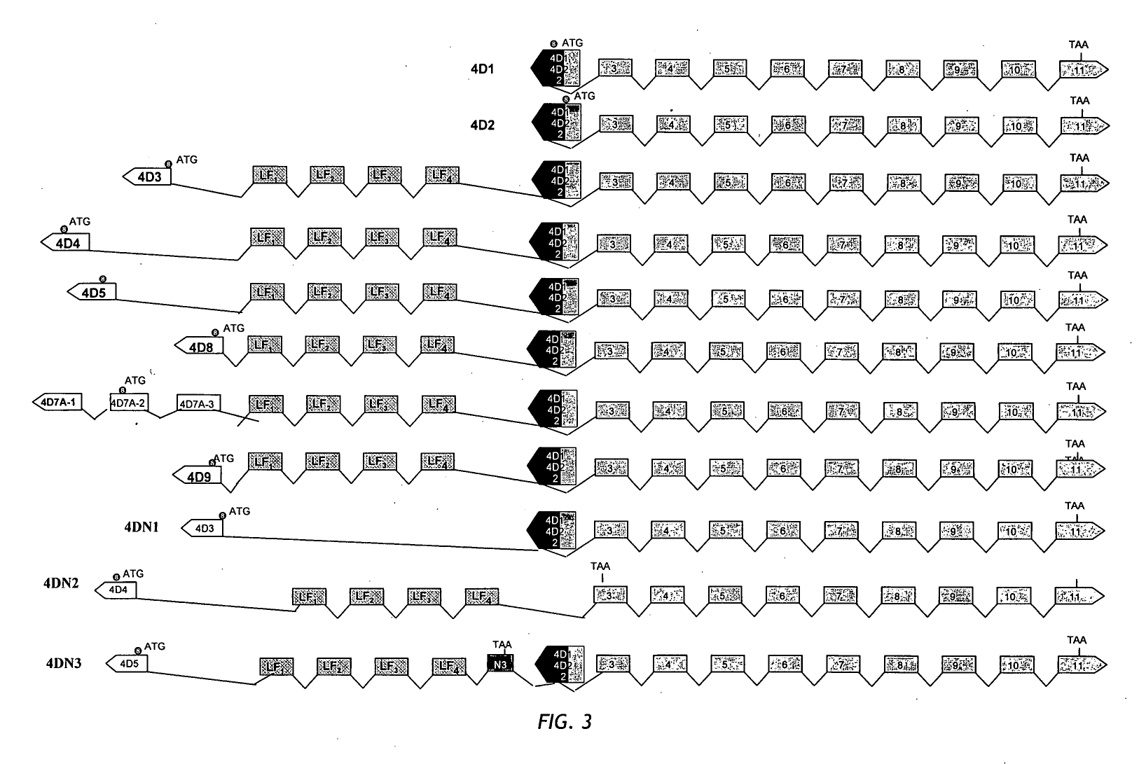

The three new exons D7A-1, D7A-2 and D7A-3 are spliced to one another and together splice onto exon LF1 forming the splice variant we named PDE4D7 (FIG. 3). Exon D7-1 is non-coding. Exons D8 and D9 are spliced by themselves onto exon LF1 forming two splice variants we named PDE4D8 and PDE4D9, respectively (FIG. 3).

In terms of genomic structure, the D7A exon extends the 5′ end of PDE4D by 590,000 bp, and the D8 and D9 exons lie between exons D3 and LF1(physical position of exons presented in Table 2C). The new PDE4D7 isoform has an open reading frame extending into LF1, resulting in additional 91 amino acids at the N-terminus of the predicted protein. The D8 and D9 5′ exons contain a long 5′ UTR, followed by an ATG near the end of the exons that extends an ORF into LF1 resulting in a novel N-terminal segments of 22 and 30 amino acids in the PDE4D8 and PDE4D9 predicted proteins, respectively. The new splice variants were verified by RT-PCR on different cDNA tissue panels and subsequent cloning and sequencing of the products.

The PDE4D gene encodes at least eight different isoforms. Six of the eight forms are the so-called long isoforms. Each of them has an unique N-terminal regulatory domain but they are all characterized by two highly conserved regions found in all PDE4 subfamilies, i.e., upstream conserved regions 1 and 2 (UCR 1 and 2). The remaining two isoforms are the short forms, variants which lack the UCR 1.

Three PDE4D isoforms have been submitted to GenBank by Memory Pharmaceuticals on Sep. 16, 2002 and Dec. 17, 2002, under accession numbers AF536975 (isoform named PDE4D6), AF536976 (named PDE4D7) and AF536977 (named PDE4D8). See also PCT WO 01/00851, published Jan. 4, 2001. The sequence AF536977 corresponds to our earlier reported PDE4D6 isoform and AF536976 corresponds partly to our earlier reported PDE4D7 isoform, however the first untranslated exon we named D7-1 is missing from this sequence. The sequence AF536975 is a new short PDE4D isoform. We have therefore changed the isoform names accordingly herein as follows: PDE4D6 is now called PDE4D8, PDE4D7 is now called PDE4D7 and PDE4D8 is now called PDE4D9. We have submitted the new PDE4D splice variants, PDE4D7 and PDE4D9 to GenBank (Accession numbers AY245866 and AY245867, respectively).

We have in addition identified 17 putative exons upstream of LF1, based on ESTs, mouse homologies and GeneMiner exon predictions. Primers designed from these exons were used in conjunction with primers from LF1 and exon3 for RT PCR in the hope of identifying novel exons. Novel exons were in turn used to design primers for various RT-PCR reactions. We also used 5′RACE primers, designed from the known exons upstream of LF1. We have to date identified 14 new exons, including exons belonging to UniGene Cluster Hs.343602 that have now been connected to LF1.

For the 5′ RACE reactions we used cDNA made from heart, SkNAS (neuroblastoma cell line) and HVAEnd 5050 (endothelial cell line). For RT-PCR reactions a number of cDNAs made of total RNA were used (see below)

Novel exons in Table 10A are in italics; previously know PDE4D exons in white. Exon 3 of EST AW272330 is included on the table as a representative of the 3′ of ESTs from UniGene cluster Hs 343602. The positions given are from SEQ ID NO: 1. Note the different splicing of 4D9-3.2, 4D9-3.1, and AW272330exon3. Total RNA was isolated from HeLa, SkNAs and Jurkat 77 cell cultures according to manual, using the TRIZOL® reagent provided by GibcoBRL. We used the GeneRacer™, ThermoZyme™ and TOPO TA cloning® (containing pCR®2.1-TOPO®) kits from Invitrogen following the manufacturer's protocol.

TABLE 10A

| | | | | supported by |
|---|---|---|---|---|
| exon # | EXON | SEQ ID 1 | SEQ ID 1 | EST(s) |
| 1 | 4D7-A | 108127 | 108217 | Yes |
| 2 | PDE4D7-1 | 142207 | 142328 | PDE4D |
| 3 | 4D7-4 | 257650 | 257705 | Yes |
| 4 | 4D7-8 | 288224 | 288393 | No |
| 5 | 4D7-B | 295203 | 295251 | No |
| 6 | 4D7-5 | 352169 | 352317 | No |
| 7 | 4D7-6 | 441914 | 442036 | No |
| 8 | PDE4D7-2 | 444645 | 444775 | PDE4D |
| 9 | 4D7-C | 482438 | 482719 | No |
| 10 | 4D7-7 | 597399 | 597534 | Yes |
| 11 | 4D7-9 | 626020 | 626092 | No |
| 12 | PDE4D7-3 | 641649 | 641878 | PDE4D |
| 13 | PDE4D4 | 736254 | 737226 | PDE4D |
| 14 | PDE4D5 | 861791 | 862202 | PDE4D |
| 15 | PDE4D3 | 1044051 | 1044190 | PDE4D |
| 16 | 4D9-1 | 1069544 | 1069629 | Yes |
| 17 | 4D9-2 | 1069936 | 1069993 | No |
| 18 | 4D9-3.2 | 1071661 | 1071795 | Yes |
| | 4D9-3.1 | 1071668 | 1071795 | |
| | AW272330exon3 | 1071668 | 1071901 | |
| 19 | 4D9-4 | 1121821 | 1121892 | No |
| 20 | 4D9-5 | 1247621 | 1247696 | No |
| 21 | PDE4D8 | 1273404 | 1273709 | PDE4D |
| 22 | PDE4D9 | 1354347 | 1355128 | PDE4D |
| | LF1exon | 1414511 | 1414702 | PDE4D |

Sequence of New Exons:

(SEQ ID NO: 88)

>4D7-A
GGCCTCGAGCAGAACTTCCCATTTGAGTGGGACCAAGAAGAGCATACAAA
GCTGAAATGTTCTCCAGAAGTTGATTTCCAATGGGGATAAA

(SEQ ID NO: 89)

>4D7-4_From Forward Primer
TGATTACAGGTTTTAGAGAAGAGGAACAATGCTTCCTCTGAGCCTGAAGA
AAAGAA

(SEQ ID NO: 90)

>4D7-8
AGTTCTGACCATGTCCTGTGTCACTCTCAAGCAGAGATTGAAAATGACAT
TCGTCCTTTACTTGTTCCAAGGAAGCAAACATTTTATAGTTTGAAACTGT
TTCTCTTGCATTTGCTTTGCAAGAGGTTTGCAGAAGTTAAGCCTCATGGA
GTCTTCTCTCCTTAACTTAA

(SEQ ID NO: 91)

>4D7-B
TGTGAAGAATTTGGAAATTGCAAGGAGCATGGGAAGGAGATGATTTGGG

(SEQ ID NO: 92)

>4D7-5
GAATGAAGAGGAAATCAAGACATACTTAGATAAAAACAGATTATCACCAG
GAGATCTGCTGTAAAAGAATGGCTAAAGGAAGTTAGCTAAGCAGAAAGGA
AGTAACATAAAAAGGAACCTTGGAACATCAGGGAGGACAAAAGAACATG

(SEQ ID NO: 93)

>4D7-C
TTTCTCTTTCTCCAATCACTCACTCTGGAGGCAGCTAGCTGTCAACTCAC
AAAGACACTCAAGCAGCCTATGGAAGAAGGCCACATGGTAAAATATGGAG
GCCTCCAGCCAACAGTCAGCAAGGAACTGAGACAAGTCAACAACCATGTG
AGTGACTCGAGAAGTGCTTCTCTAGCTCCAGTTGAGACTTGCAGTAGCAG
CAGCCTCAGCTGGCGGCTTGACTGCAATCTCTTGAGAGACCCTAAGCTCT
CCTGAATTCTTGATCCTTAGAAACTGTGTGAG

(SEQ ID NO: 94)

>4D7-6
GGTCTAGCTGTGTCCCAGAGAGCAACTTCCCTTTTCAAGGCAGCCCACTC
TGTGTGATGCTTTTTCCTAGGTATGGGCAACCCATCCCTCCTAGGGTGAA
AACTTCGCTGTTGCTAGTTCCAG

(SEQ ID NO: 95)

>4D7-7
AATGATGCCGTATTATTCTCCTGACCTAACTTCAAAGAAATAAAGAGTTT
GCAAGAAGAACTGCAGTTCTTCAAAGTACGCAATATGGATTTCCAAGATG
AATGTAGTTTCTCTCTCTGAGGAATTCTGAACAGTG

(SEQ ID NO: 96)

>4D7-9
GACTTGAGCATCTGAAGATTTTGGTTTCTGCAGAGGGTGGGAAAGGTTGA
ACCAATCCCCCATGGATACCAAG

(SEQ ID NO: 97)

>4D9-1
GGCTTTCCAGATCCCTGAAGATAAAATACAAACTCTCCAACAAGACCTTT
TGGCCATCAGGAACGCAGCACCTGGCTCTCTCACTA

(SEQ ID NO: 98)

>4D9-2
AAAGTCGCAGAGATAGCGGAGAACAAGAACCAGATCTCACAGTCATGGTG
CCAAAAGA

(SEQ ID NO: 99)

>4D9-3.1
CTGTTACCCTAGCATGACTGCTTCAGCGAAGAGATAAGAGCTTCTTTGAC
TTTTTCCACTGGAATTTTTCATGCCAGAAGAAATTGAACATGTGAGCCTG
GTGTCTGGAAGAGTAGCCTGGATTTATG

(SEQ ID NO: 100)

>4D9-3.2
AATTCAGCTGTTACCCTAGCATGACTGCTTCAGCGAAGAGATAAGAGCTT
CTTTGACTTTTTCCACTGGAATTTTTCATGCCAGAAGAAATTGAACATGT
GAGCCTGGTGTCTGGAAGAGTAGCCTGGATTTATG

(SEQ ID NO: 101)

>4D9-4
TTCCTTGATAGTTCCAATATCTGTAATCTTGTTGGTCTACCTGTGCAGTT
TATTCCACTGATTGTCTCTCAG

(SEQ ID NO: 102)

>4D9-5
GCGAAAATACTGAGGCTCAACAGACATAAAATGGCTTGAGTTACCAGGCT
ACAGTAGAACTAGGATTTCAGTCCAG

Splicing of the Exons as identified by RT-PCR/RACE
New exons are in italics.
RT4D7: 4D7-1+4D7-2+4D7-3+LF1
RT1: 4D7-1+4D7-8+4D7-2+4D7-3+LF1
RT2: 4D7-4+4D7-2+4D7-9+4D7-3+LF1
RT3: 4D7-2+4D7-3+LF1
RT4: 4D7-1+4D7-2+4D7-7+4D7-3+4D9-1+4D9-3.1
RT5: 4D7-1+4D7-2+4D7-3+4D9-2+4D9-3.1
Race6: 4D7-A+4D7-B+4D7-2+4D7-C+4D7-3
RT7: 4D7-1+4D7-4
RT8: 4D7-1+4D7-5+4D7-2
RT9: 4D7-1+4D7-6+4D7-2
RT10: 4D9-1+4D9-3.1+LF1
RT11: 4D9-2+4D9-3.2+LF1
RT12: 4D9-3+4D9-4+LF1
RT13: 4D9-3+4D9-4+4D9-5+LF1
RT14: 4D9-3+LF1

Detection of variants in cDNA from various tissues:

TABLE 10B

| | RT4D7 | RT1 | RT2 | RT3 | RT4 | RT5 | RT6 | RT7 | RT8 | RT9 | RT10 | RT11 | RT12 | RT13 | RT14 |
|---|---|---|---|---|---|---|---|---|---|---|---|---|---|---|---|
| Bone Marrow | − | − | − | * | − | − | n | n | n | n | n | − | − | − | − |
| Brain | + | − | − | * | − | − | n | n | n | n | n | n | − | + | − |
| fetal Brain | + | − | − | * | − | − | n | n | n | n | n | n | − | − | − |
| colon | + | + | − | * | − | − | n | n | n | n | n | n | − | − | − |
| Heart | − | − | − | − | − | − | − | − | − | − | − | − | − | − | + |
| HVAEend 5050 | − | − | n | n | n | n | + | − | − | − | + | + | n | n | + |
| Kidney | + | − | + | + | − | − | n | n | n | n | n | n | − | − | − |
| Liver | − | − | − | * | − | − | n | n | n | n | n | − | − | − | − |
| Placenta | − | − | − | * | − | − | n | n | n | n | n | − | − | − | − |
| Prostate | + | − | − | * | + | − | n | n | n | n | n | n | − | − | * |
| Salivary gland | + | − | − | * | − | − | n | n | n | n | n | n | − | − | − |
| Skeletal Muscle | − | − | − | − | − | − | n | n | n | n | n | n | + | − | + |
| SkNAS cell line | + | − | n | * | n | n | − | + | + | + | − | − | n | n | − |
| Spinal Cord | − | − | − | − | − | − | n | n | n | n | n | n | − | − | * |
| Spleen | − | − | − | * | − | − | n | n | n | n | n | n | − | − | + |
| Testis | − | − | − | − | − | − | n | n | n | n | n | n | − | − | * |
| Thymus | + | − | − | * | − | − | n | n | n | n | n | n | − | − | − |
| Thyroid | + | − | − | * | − | + | n | n | n | n | n | n | − | − | + |
| Trachea | + | − | − | * | − | − | n | n | n | n | n | n | − | − | − |
| Uterus | − | − | − | − | − | − | n | n | n | n | n | n | − | − | + |
| other# | − | − | − | − | − | − | n | n | n | n | n | n | − | − | − |

+ = present and verified by sequencing
* = product of the correct size present; not yet verified by sequencing
− = not detected
n = not checked
#These are: Adrenal Gland, Fetal Liver, Cerebellum, Lung, Small Intestine.

Two of the variants that are more widely expressed appear to be mutually exclusive: 4D7 [with 4D7-1 as first exon] was detected in 10 cDNAs while RT14 is found in 9cDNAs. Of these thyroid and prostate are the only tissues common to both variants.

The 13 new RT and RACE variants presented above (we had previously described the 4D7 variant), do not add any new translated sequence. The RT1 product is expected o be the same as the 4D7 putative protein. In variants RT2 and Race6 the exons between 4D7-2 and 4D7-3 interfere with the ORF with the first AUG and ORF being just inside LF1. Similarly Exon 4D9-3 contains stop codons in all 3 reading frames and Variants RT10, 11,12,13, and 14 having their ATG initiation codon inside LF1. It is not clear whether variants RT4 and RT5, which contain exon 4D9-3 extend to LF1 or have their 3′ at 4D9-3 (the latter possibility is supported by the EST data).

It is noteworthy that all variants except 4D7, RT3 and RT14 have been observed only in one of the cDNAs. Although all the new exons (except 4D9-3.1) have an AG/GT splice signal, it is plausible that these variants represent rare or aberrant events with little physiological significance.

The following exons contain Alu repetitive element sequences: 4D7-5 and 4D7-C. The gene specific reverse (3′) primer was designed for PDE4D exon LF1 (5′ GGCAATGGAGGAGTTCCGGGACA TA-3′; SEQ ID NO: 87 origin from Homo sapiens).

A contig for the incomplete genomic sequence of the PDE4D gene was submitted by others in November 2000 (GenBank entry NT_{—}023193 by International Human Genome Project collaborators). The size of the contig is 614 481 bp (including gaps) whereas our novel genomic sequence for the whole PDE4D region (i.e., from the first exon for PDE4D variant) is close to 1,690,000 bp and contains no gaps. The contig NT_{—}023193 comprises only 11 exons of the PDE4D gene (in FIG. 3, exons 4D1/D2-11) and the 5′ differently spliced exons are missing in the contig (in FIG. 3, exons D4, D5, D3, D8, D9, D7A-1, D7A-2, D7A-3, LF1, LF2, LF3 and LF4).

TABLE 13
New Isoforms

| Isoform | | | | |
|---|---|---|---|---|
| Name | Exon | | Size | Cell line |
| PDE4D7 | D7-1 | 5′ | 122 bp | SKNAS |
| PDE4D7 | D7-2 | Internal | 131 bp | SKNAS |
| PDE4D7 | D7-3 | Internal | 230 bp | SKNAS |
| PDE4D9¹ | D9 | 5′ | 782 bp | HeLa |

¹Formerly referred to in previous applications as PDE4D8

The sequences are as follows:

D7A-1:
ATAGTTGGCGTACCCTGAGGCCTGCCAGTTCCTGCCTTAATGCATATGTA
GTCGTAATTGAGTTCTGACACGGCCTTGGATGTTTCTGTCCTAAATAGCT
GACATTGCATCTTCAAGACTGT
D7A-2:
CATTCCAGTTGGCTTTTGAGTGGATACGTGCAGTGAGATCATTGACACTG
GAAACACTAGTTCCCATTTTAATTACTTAAAACACCACGATGAAAAGAAA
TACCTGTGATTTGCTTTCTCGGAGCAAAAGT
D7A-3:
GCCTCTGAGGAAACACTACATTCCAGTAATGAAGAGGAAGACCCTTTCCG
CGGAATGGAACCCTATCTTGTCCGGAGACTTTCATGTCGCAATATTCAGC
TTCCCCCTCTCGCCTTCAGACAGTTGGAACAAGCTGACTTGAAAAGTGAA
TCAGAGAACATTCAACGACCAACCAGCCTCCCCCTGAAGATTCTGCCGCT
GATTGCTATCACTTCTGCAGAATCCAGTGG (SEQ ID NO: 11;
includes D7A-1, D7A-2 and D7A-3)

New predicted amino-terminal protein sequence from above (PDE4D7):

(SEQ ID NO: 12)

MKRNTCDLLSRSKSASEETLIJSSNEEEDPFRGMEPYLVRRLSCRNIQLP
PLAFRQLEQADLKSESENIQRPTSLPLKILPLIAITSAESS
(90 amino acids)

(SEQ ID NO: 13)

D9:
TTCTCACTGCCCTGCGGTGTTTTGAACTGCCTTCTTACAGACGTCATACA
GCCCTTGAGGAATAGTTTCTGCCTGGTGAGATTGAATGATAGTTCTCATT
CACAAAACCCTGGATTCTAAGCAGGGACACACAGAAATTACTTTCGCAGG
TAAATCAGCCCACCCAGCCAAAGTGTGGAGAGATTTGTTCCTFTGGCTGA
CTTCTTTGCTCCACGGAGAGGAGTGTTTTCCTGTGCTTGCCCTGAAATGG
AACTTCCTTGACAGCTCTCCCGTGTTACAGTACCTCCCGGTCATTTTCTT
TTTCTCTCTCTCTACCTGCGCTCTTCGAGTGTCAGAAACCTTTAAAGCTG
TTACTATGGAATTGCAAAAAAGAGATCAAGTGACTCTTTCACTATGCTGG
TTTCCCTTGTGACCCAGATGAAGAATCAATTCAGAATTCAGTTCCTCCCT
TGGCATTGCAAGACACAGAAGAAACTGTCACTTCCTAACAGCCTAGTACT
GGAGTAAATTCAGTATGAAGGAAGAAAGCGCTCCTGCGTGTTAGAACCTT
GCCCATGAGCTGGACCGAGGACAGGAGATGGACTCCAGGAAAATTGGATT
TCTTCAAGCAGCCTCCCTTGGAAATGGAATATCTTTAAAATCTTCTTTGC
AGAAAGACAGTTAGAATGTATTAATCAGAATAGTTGAAGACTTATTTTCC
TTTTTATTTTTTTTCAAAATGAGCATTATTATGAAGCCAAGATCCCGATC
TACAAGTTCCCTAAGGACTGCAGAGGCAGTTTG

New predicted amino-terminal protein sequence from above (PDE4D9):

(SEQ ID NO: 14)

MSIIMKPRSRSTSSLRTAEAV (21 amino acids).

TABLE 11
Publically Available SNPS; SNP ID No. from NCBI Database

| | | | | | |
|---|---|---|---|---|---|
| rs286155 | rs27960 | rs27221 | rs149079 | rs789615 | rs37708 |
| rs286156 | rs27564 | rs27653 | rs149324 | rs401207 | rs37709 |
| rs2061250 | rs27565 | rs26955 | rs153067 | rs364917 | rs789389 |
| rs286150 | rs26948 | rs26956 | rs40354 | rs404202 | rs1423247 |
| rs206789 | rs40131 | rs153031 | rs26951 | rs440607 | rs874768 |
| rs1823062 | rs26949 | rs185190 | rs153029 | rs411255 | rs2042315 |
| rs1823063 | rs26950 | rs37762 | rs27223 | rs615429 | rs918590 |
| rs1445852 | rs26954 | rs37761 | rs27222 | rs789396 | rs918591 |
| rs766119 | rs26953 | rs1423471 | rs251726 | rs37684 | rs918592 |
| rs956721 | rs152324 | rs27224 | rs1862589 | rs1445893 | rs1115372 |
| rs248910 | rs35385 | rs1645013 | rs702556 | rs37685 | rs1345782 |
| rs248912 | rs40512 | rs1423472 | rs702554 | rs1086121 | rs1363862 |
| rs187481 | rs35386 | rs27220 | rs441391 | rs42222 | rs1423248 |
| rs153152 | rs35387 | rs1423473 | rs446883 | rs37707 | rs1423246 |
| rs1862614 | rs1995780 | rs1435077 | rs159624 | rs1008709 | rs298088 |
| rs2194256 | rs1508865 | rs1369287 | rs1159470 | rs1027747 | rs298087 |
| rs889305 | rs952110 | rs1017410 | rs159622 | rs869685 | rs1421401 |
| rs2113071 | rs1533019 | rs1017409 | rs256349 | rs869686 | rs298086 |
| rs2113072 | rs2117552 | rs1435076 | rs256348 | rs924880 | rs298085 |
| rs966220 | rs1545069 | rs1435075 | rs1501640 | rs1504983 | rs298084 |
| rs966221 | rs1545070 | rs1435074 | rs600611 | rs1504982 | rs298083 |
| rs719702 | rs973700 | rs978455 | rs159621 | rs877745 | rs298073 |
| rs2113073 | rs1583434 | rs1827340 | rs159625 | rs877744 | rs298072 |
| rs2113074 | rs1347401 | rs1393083 | rs1435072 | rs2164661 | rs298071 |
| rs2113075 | rs1949017 | rs988364 | rs173945 | rs981230 | rs1421400 |
| rs1035512 | rs723962 | rs1017408 | rs256356 | rs1437124 | rs402874 |
| rs1559277 | rs1355099 | rs2053155 | rs185351 | rs746477 | rs434368 |
| rs1981848 | rs1396473 | rs181923 | rs256355 | rs893191 | rs371011 |
| rs1544788 | rs1369285 | rs1546364 | rs2067024 | rs1992112 | rs298063 |
| rs1544790 | rs1435071 | rs173942 | rs256354 | rs298102 | rs298062 |
| rs1544791 | rs1435070 | rs159616 | rs173944 | rs298101 | rs298061 |
| rs851284 | rs1435083 | rs159620 | rs256353 | rs2164660 | rs298060 |
| rs1396476 | rs991551 | rs1501641 | rs986400 | rs298100 | rs298057 |
| rs1508860 | rs1154790 | rs159619 | rs1504981 | rs298098 | rs298056 |
| rs1974850 | rs1154789 | rs159614 | rs1120533 | rs298096 | rs1370230 |
| rs2136203 | rs714291 | rs159613 | rs256351 | rs298095 | rs297975 |
| rs2174994 | rs981760 | rs159612 | rs190458 | rs298094 | rs297974 |
| rs1508863 | rs1369288 | rs159611 | rs256352 | rs298093 | rs379578 |
| rs1508859 | rs977418 | rs194368 | rs171745 | rs1362942 | rs920190 |
| rs1508864 | rs977417 | rs661576 | rs1157709 | rs1362941 | rs1865962 |
| rs1396474 | rs977416 | rs299627 | rs1910790 | rs298091 | rs298018 |
| rs1543951 | rs1529843 | rs159608 | rs1910789 | rs298090 | rs298021 |
| rs2016324 | rs1529842 | rs159609 | rs1504985 | rs298089 | rs298022 |
| rs298023 | rs2053229 | rs296406 | rs697076 | rs37575 | rs1824154 |
| rs298024 | rs295974 | rs296405 | rs294478 | rs37576 | rs2112911 |
| rs298025 | rs295973 | rs295948 | rs953302 | rs1876209 | rs1551564 |
| rs298026 | rs295972 | rs295947 | rs294479 | rs190486 | rs2034895 |
| rs298027 | rs295971 | rs295946 | rs697075 | rs447261 | rs2081092 |
| rs298028 | rs295970 | rs295945 | rs294481 | rs1506558 | rs2112910 |
| rs298029 | rs295969 | rs295944 | rs294482 | rs1108916 | rs918583 |
| rs298030 | rs295968 | rs1395334 | rs294483 | rs921942 | rs1840838 |
| rs169868 | rs295966 | rs295943 | rs702545 | rs924998 | rs1350298 |
| rs177077 | rs726652 | rs1035321 | rs294484 | rs176705 | rs1990985 |
| rs298032 | rs295965 | rs294494 | rs294485 | rs1156029 | rs1379297 |
| rs298033 | rs1307218 | rs722923 | rs294486 | rs1156028 | rs1817248 |
| rs298034 | rs1307217 | rs294495 | rs702544 | rs931857 | rs244569 |
| rs298035 | rs893190 | rs294496 | rs702543 | rs931856 | rs244568 |
| rs298042 | rs1111495 | rs294497 | rs159194 | rs931855 | rs244567 |
| rs298044 | rs295961 | rs294498 | rs40215 | rs1506557 | rs244565 |
| rs298045 | rs295960 | rs294499 | rs291118 | rs462930 | rs185417 |
| rs298046 | rs295959 | rs294500 | rs1506560 | rs458953 | rs258128 |
| rs298048 | rs295958 | rs294501 | rs37569 | rs174039 | rs258127 |
| rs298049 | rs296410 | rs294503 | rs291119 | rs2174624 | rs258125 |
| rs298050 | rs295957 | rs295936 | rs37571 | rs2135480 | rs1348710 |
| rs298051 | rs295956 | rs1395336 | rs1870077 | rs992726 | rs1348709 |
| rs298052 | rs295955 | rs1395337 | rs159195 | rs294474 | rs1971061 |
| rs298053 | rs295954 | rs294492 | rs37572 | rs294475 | rs1541673 |
| rs190936 | rs295949 | rs159196 | rs37573 | rs988827 | rs1541672 |
| rs298017 | rs295980 | rs159197 | rs167161 | rs988828 | rs258112 |
| rs298016 | rs295979 | rs172362 | rs37574 | rs1350297 | rs258111 |
| rs298015 | rs295978 | rs37579 | rs1506562 | rs1457110 | rs171800 |
| rs298014 | rs1154587 | rs721784 | rs291122 | rs1457111 | rs187716 |
| rs258110 | rs424839 | rs1118965 | rs35266 | rs255652 | rs26709 |
| rs258109 | rs370891 | rs154028 | rs39672 | rs255650 | rs26710 |
| rs258108 | rs434183 | rs151802 | rs958851 | rs255649 | rs28055 |
| rs258107 | rs444552 | rs244580 | rs244576 | rs2194210 | rs26711 |
| rs665836 | rs433565 | rs1457145 | rs244575 | rs255648 | rs27723 |
| rs392901 | rs1445918 | rs244579 | rs244573 | rs255647 | rs27185 |
| rs383444 | rs441817 | rs255812 | rs35258 | rs154221 | rs27695 |
| rs662643 | rs433161 | rs154029 | rs35259 | rs256752 | rs1445954 |
| rs670169 | rs428059 | rs185333 | rs40121 | rs256120 | rs27549 |
| rs525099 | rs434422 | rs35289 | rs35261 | rs255635 | rs455969 |
| rs669240 | rs427433 | rs35288 | rs35264 | rs185325 | rs26712 |
| rs381755 | rs391377 | rs35287 | rs40122 | rs26686 | rs1867711 |
| rs454702 | rs414746 | rs35286 | rs35265 | rs1031197 | rs1867712 |
| rs443191 | rs187368 | rs35285 | rs35255 | rs1031198 | rs26713 |
| rs380118 | rs244593 | rs35284 | rs721826 | rs27183 | rs26714 |
| rs2168649 | rs244592 | rs35283 | rs244570 | rs28044 | rs27547 |
| rs371775 | rs244591 | rs35282 | rs27171 | rs27182 | rs26715 |
| rs378970 | rs244590 | rs35281 | rs1824159 | rs545611 | rs27949 |
| rs401013 | rs181736 | rs35280 | rs27170 | rs649476 | rs26700 |
| rs427748 | rs193447 | rs35279 | rs27169 | rs1664896 | rs1306348 |
| rs427740 | rs2028842 | rs35278 | rs27168 | rs149106 | rs35309 |
| rs378869 | rs2028841 | rs40126 | rs2013979 | rs1374028 | rs27691 |
| rs1902609 | rs1823068 | rs35277 | rs889231 | rs531105 | rs35310 |
| rs389324 | rs1823067 | rs35276 | rs2014012 | rs27184 | rs26689 |
| rs387647 | rs1823066 | rs35275 | rs37353 | rs1445951 | rs27187 |
| rs377451 | rs244588 | rs40125 | rs187645 | rs1947090 | rs1445948 |
| rs403695 | rs168641 | rs35274 | rs1809012 | rs26708 | rs26687 |
| rs403672 | rs2059175 | rs244577 | rs187644 | rs2112959 | rs166260 |
| rs372309 | rs2059174 | rs35267 | rs153981 | rs1445953 | rs149506 |
| rs27722 | rs1664886 | rs1559251 | rs1553113 | | |
| rs26695 | rs1867724 | rs1345791 | rs1353748 | | |
| rs27773 | rs1445947 | rs1345792 | rs1498606 | | |
| rs1471429 | rs42470 | rs1345793 | rs1353747 | | |
| rs1471430 | rs1423308 | rs1105577 | rs1006431 | | |
| rs26705 | rs27174 | rs1960 | rs1948651 | | |
| rs28054 | rs168834 | rs1824788 | rs1498605 | | |
| rs26703 | rs27727 | rs1862563 | rs1498604 | | |
| rs27898 | rs27172 | rs1551939 | rs1498603 | | |
| rs722010 | rs676449 | rs1038080 | rs1995166 | | |
| rs27957 | rs27186 | rs997421 | rs1498602 | | |
| rs26702 | rs2112957 | rs1014317 | rs1077183 | | |
| rs27548 | rs1023814 | rs2059191 | rs1078368 | | |
| rs26701 | rs27175 | rs1551938 | rs1874857 | | |
| rs27188 | rs1445950 | rs1186170 | rs1874858 | | |
| rs27189 | rs2021384 | rs986067 | rs1909294 | | |
| rs149084 | rs736736 | rs954740 | rs1546221 | | |
| rs153968 | rs745813 | rs1363882 | rs2055295 | | |
| rs464787 | rs889229 | rs1353749 | rs1391648 | | |
| rs153978 | rs1077978 | rs1391651 | rs2055298 | | |
| rs464311 | rs2081106 | rs1391650 | rs1472456 | | |
| rs149108 | rs1559252 | rs1391649 | rs1553114 | | |
| rs153980 | rs2054443 | rs1391652 | rs1542842 | | |
| rs153961 | rs922437 | rs950446 | rs1498611 | | |
| rs1867725 | rs922436 | rs950447 | rs1532520 | | |
| rs153965 | rs922435 | rs1498599 | | | |
| rs153966 | rs922434 | rs1498601 | | | |
| rs1988803 | rs716908 | rs1498609 | | | |
| rs467300 | rs1971940 | rs1498608 | | | |

TABLE 12
New SNPs identified by deCODE

| Position | Variation | AA Change | Exon |
|---|---|---|---|
| 135641 | T/A | | |
| 142780 | A/G | | |
| 732790 | G/T | | |
| 735966 | C/A | | |
| 736226 | A/G | | |
| 736516 | C/T | | |
| 850001 | G/A | | |
| 852776 | A/C | | |
| 853079 | G/T | | |
| 853575 | C/A | | |
| 856468 | A/G | | |
| 860845 | A/G | | |
| 870924 | A/G | | |
| 1027267 | T/C | | |
| 1027643 | T/G | | |
| 1027757 | T/C | | |
| 1028146 | T/A | | |
| 1037657 | A/C | | |
| 1044016 | G/A | | |
| 1044045 | C/T | | |
| 1254737 | T/C | | |
| 1254849 | T/C | | |
| 1255763 | G/T | | |
| 1257206 | A/G | | |
| 1258161 | T/C | | |
| 1268007 | A/G | | |
| 1268187 | C/T | | |
| 1268553 | A/G | | |
| 1272669 | G/A | | |
| 1272910 | A/G | | |
| 1273023 | G/A | | |
| 1273220 | A/G | | |
| 1273240 | A/G | | |
| 1273543 | C/T | | |
| 1288439 | G/A | | |
| 1289730 | T/A | | |
| 1290176 | G/A | | |
| 1293745 | T/C | | |
| 1344605 | A/G | | |
| 1344864 | G/A | | |
| 1345135 | C/G | | |
| 1345286 | A/G | | |
| 1346112 | C/T | | |
| 1352976 | A/T | | |
| 1354291 | T/C | | |
| 1354377 | C/T | | |
| 1354554 | C/A | | |
| 1354675 | T/C | | |
| 1355114 | T/C | | |
| 1355693 | A/G | | |
| 1357081 | A/G | | |
| 1362985 | T/G | | |
| 1363021 | C/T | | |
| 1363827 | C/T | | |
| 1363911 | G/A | | |
| 1364061 | C/T | | |
| 1364066 | T/A | | |
| 1367904 | A/G | | |
| 1368193 | T/C | | |
| 1368217 | G/C | | |
| 1373349 | C/T | | |
| 1373384 | A/G | | |
| 1373415 | T/C | | |
| 1373979 | T/G | | |
| 1376149 | G/A | | |
| 1384931 | A/C | | |
| 1385093 | A/T | | |
| 1385107 | G/A | | |
| 1385445 | T/C | | |
| 1391418 | G/C | | |
| 1409210 | C/A | | |
| 1414804 | C/T | | |
| 1428284 | T/C | | |
| 1431800 | A/T | | |
| 1449904 | A/T | | |
| 1574301 | C/G | | |
| 1574615 | C/T | | |
| 1575634 | A/T | | |
| 1580088 | G/A | | |
| 1581078 | G/A | | |
| 1582418 | T/A | | |
| 1584580 | A/C | | |
| 1585955 | G/T | | |
| 1590608 | T/C | | |
| 1590672 | A/G | | |
| 1590673 | G/T | | |
| 1590837 | G/A | | |
| 1590936 | C/A | | |
| 1591011 | G/A | | |
| 1591047 | C/T | | |
| 1591306 | C/A | Pro->Thr | D1 |
| 1591583 | T/C | | |
| 1594788 | C/A | | |
| 1594994 | G/A | | |
| 1601831 | C/T | | |
| 1636902 | T/C | | |
| 1638550 | A/C | Lys->Thr | exon 4 |
| 1640663 | T/C | | |
| 1641954 | C/T | | |
| 1641960 | C/T | | |
| 1653881 | G/A | | |
| 1655748 | G/A | | |
| 91470 | G/A | | |

Discussion of Example 2:

Here we present the first complete genomic sequence of human PDE4D, two novel mRNA/protein isoforms of PDE4D and their corresponding exons, and the intron-exon structure of known and novel isoforms. The basis for phosphodiesterases is the mammalian homolog of the “dunce” gene in Drosophila melanogaster, implicated in learning and memory (Davis, R. L. and B. Dauwalder, Trends Genet., 7(7):224-229 (1991)). PDEs are members of a large superfamily of isoenzymes subdivided into 9 and possibly 10 distinct families (Conti, M. and S. L. Jin, Prog. Nucleic Acid Res. Mol. Biol., 63:1-38 (1999)), with several genes in each family and more than one isoform for each gene. The significance of the diversity of PDEs is not known but many of the isoforms differ in their biochemical properties, phosphorylation, intracellular targeting, protein-protein interactions and patterns of expression in tissues, which suggests that each of the various isoforms might have distinct functions (Bolger, G. B., Cell Signal, 6(8):851-859 (1994); Conti, M., et al., Endocr. Rev., 16(3):370-378 (1995)).

There are four genes that encode the type 5 PDEs (PDE4A, PDE4B, PDE4C and PDE4D), which is a group of enzymes characterized by high affinity for cAMP. The gene for PDE4D was assigned to human chromosome 5q12 (Milatovich, A., et al., Somat. Cell Mol. Genet., 20(2):75-86 (1994); Szpirer, C., et al., Cytogenet. Cell Genet., 69(1-2):22-14 (1995)) and 5 distinct splice variants have been characterized (the short forms PDE4D1, PDE4D2 and the long forms PDE4D3, PDE4D4, and PDE4D5) (Bolger, G. B., et al., Biochem. J., 328(Pt. 2):539-548 (1997)) (FIG. 3). The sequence of the human PDE4D variants show a high degree of homology to the PDE4Ds expressed in mouse and rat. The pattern of splicing and different promoter usage is highly conserved during evolution indicating an important physiological role (Nemoz, G., et al., FEBS Lett., 384(1):97-102 (1996)). The PDE4D variants are generated at two major boundaries present in the gene. The first boundary corresponds to the junction of exon 2. Differential splicing in this region generates the 2 short variants PDE4D1 (586 a.a.) and PDE4D2 (508 a.a.) (FIG. 3). This splicing boundary is conserved in mouse, rat and between different human PDE4 genes. The splicing variant PDE4D2 is generated by the removal of 256 bp from the PDE4D1 sequence. The initiation codon in the PDE4D2 variant lies within exon D1/D2. Data demonstrates that the expression of the short PDE4D variants is under the control of an internal promoter regulated by cAMP (Vicini, E. and M. Conti, Mol. Endocrinol., 11(7):839-850 (1997)). The second major splicing boundary is also conserved during evolution and is identical to that described in the Drosophila dunce gene. Splicing occurs at the intron/exon boundary at the LF1 exon (FIG. 3).

PDE Function

The PDEs serve at least four major functions in the cell. They can (1) act as effector of signal transduction by interacting with receptors and G-proteins; (2) integrate the cyclic nucleotide-dependent pathway with other signal transduction pathways; (3) function as homeostatic regulators, playing a role in feedback mechanisms controlling cyclic nucleotide levels during hormone and neurotransmitter stimulation; (4) play an important role in controlling the diffusion of cyclic nucleotides and in creating subcellular domains or channeling cyclic nucleotide signaling (Conti, M. and S. L. Jin, Prog. Nucleic Acid Res. Mol. Biol., 63:1-38. (1999)). Inhibition of PDE has long been recognized as an effective pharmacological strategy to alter intracellular cyclic nucleotide levels (Flamm, E. S., et al., Arch. Neurol., 32(8):569-71 (1975)).

It has been reported that PDE4 is the predominant isozyme regulating vascular tone mediated by cAMP hydrolysis in cerebral vessels (Willette, R. N., et al., J. Cereb. Blood Flow Metab., 17(2):210-9 (1997)).

A recent study on mice with targeted disruption of PDE4D gene (Hansen, G., et al., Proc. Natl. Acad. Sci. USA, 97(12):6751-6 (2000)) has demonstrated a crucial role of PDE4D in the control of smooth muscle contraction and muscarinic cholinergic receptor signaling but not in the control of airway inflammation. The lung phenotype of the PDE4D−/− mice demonstrates that this gene plays a nonredundant role in cAMP homeostasis. There is a significant reduction in PDE activity and an increase in resting and stimulated cAMP levels in the lung, indicating that other PDE4s (or other PDEs) are not up-regulated and cannot compensate for the loss of PDE4D. These findings support that PDE4D serves a unique, nonoverlapping functions in cell signalling.

No clear link between an established inherited disorder and known PDE loci has emerged, with the exception of PDE6. Inhibitors of PDEs have been shown to affect airway responsiveness and pulmonary allergic inflammation (Schudt, C., et al., Pulm. Pharmacol. Ther., 12(2): 123-9 (1999)). There are reports suggesting that altered PDE4 function may be linked to nephrogenic diabetes insipidus (Takeda, S., et al., Endocrinology, 129(1):287-94 (1991)) or atopic dermatitis (Chan, S. C., et al., J. Allergy Clin. Immunol., 91(6):1179-88 (1993)), however no mutations have been identified. It has also been reported that vasorelaxation modulated by PDE4 (not mentioned whether it is A, B, C or D gene family) is compromised in chronic cerebral vasospasm associated with subarachnoid hemorrhage (Willette, R. N., et al., J. Cereb. Blood Flow Metab., 17(2):210-9 (1997)). PDE4D itself has not been linked to stroke before.

PDE4D Expression and Cellular Localization

PDE4Ds are expressed in human peripheral mononuclear cells (Nemoz, G., et al., FEBS Lett, 384(1):97-102 (1996)), brain (Bolger, G., et al., Mol. Cell Biol., 13(10):6558-71 (1993)), heart (Kostic, M. M., et al., J. Mol. Cell Cardiol., 29(11):3135-46 (1997)) and vascular smooth muscle cells (Liu, H. and D. H. Maurice, J. Biol. Chem., 274(15):10557-65 (1999)).

Immunoblotting of rat brain has shown that the PDE4D3, PDE4D4 and PDE4D5 proteins are present in brain (Bolger, G. B., et al., Biochem. J., 328(Pt 2):539-48 (1997)) and are expressed in cortex and cerebellum from rat (Iona, S., et al., Mol. Pharmacol., 53(1):23-32 (1998)). These proteins were recovered mostly or exclusively in the particulate fraction suggesting that these forms may be targeted to insoluble cellular structures. In addition a 68 kDa protein was detected which could represent PDE4D 1, PDE4D2 or both. To verify this RT-PCR was performed on mRNA from rat brain and the results showed that transcripts for PDE4D1 and 2 were present. Their data also suggests that the N-terminal regions of the PDE4D3-5, derived from alternatively spliced regions of their mRNAs, are important in determining their subcellular localization activity and differential sensitivity to inhibitors and there are indications that there is a propensity for the long PDE4D isoforms to interact with particulate fraction of the cell.

### Example 3

### PDE4D Isoform Expression

Expression Analysis in EBV Transformed B Cell Lines

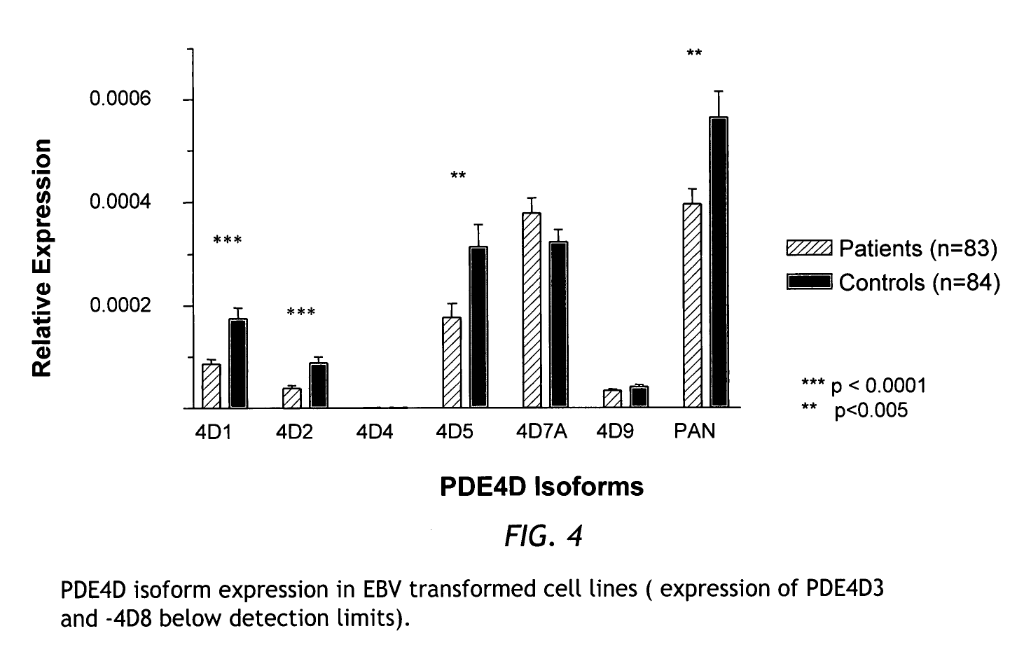

As a functional mutation in the known coding exons of PDE4D was not identified, gene expression was next studied to determine if the genetic association to stroke relates to regulation of its expression levels. In order to test this, we chose to use cell lines instead of blood or tissues for these studies because expression analysis of cell lines is not confounded by the presence of multiple cell types. Cell types may express PDE4D at different levels so it is generally more reliable to quantify expression in cell lines than tissues. Isoform-specific kinetic PCR analysis was carried out on EBV transformed B cell lines to quantify each isoform in 83 stroke patients and 84 controls. These patients were not selected for this analysis based on any specific subtype of stroke. The majority of the patients had ischemic stroke and 38% of them had carotid or cardiogenic cause of stroke. Overall the total PDE4D message level as assessed by amplification across exons present in all isoforms (PAN), was significantly lower in patients than in controls (p value<0.005). This decrease was due primarily to lower expression of the isoforms, PDE4D1, PDE4D2 and PDE4D5 (FIG. 4).

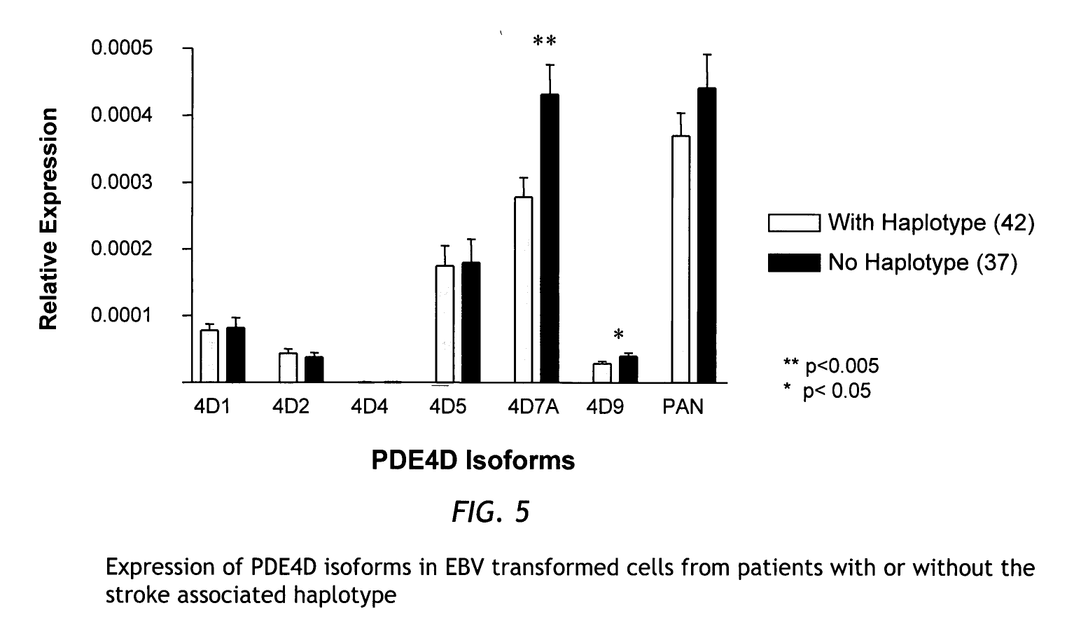

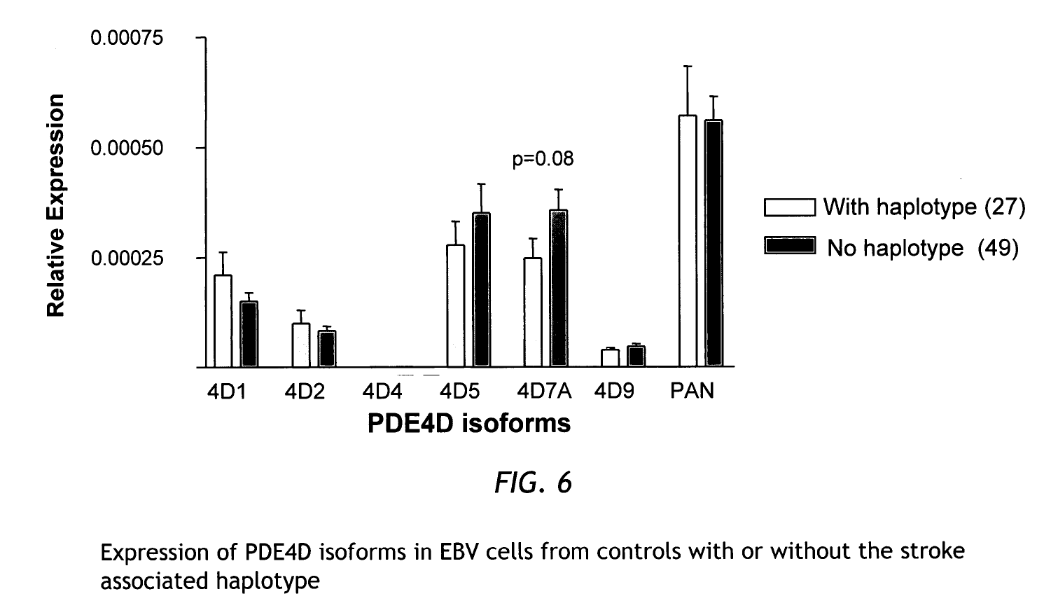

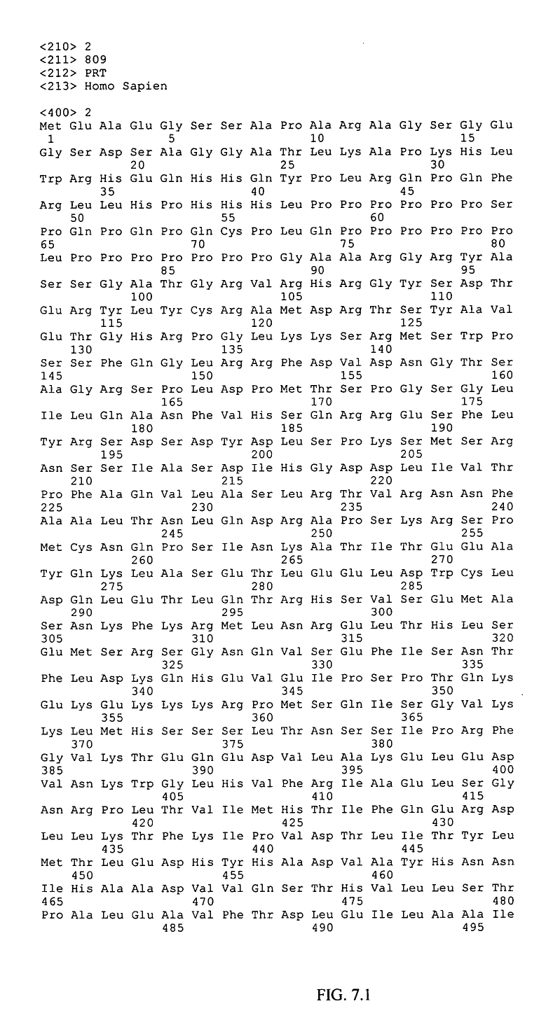

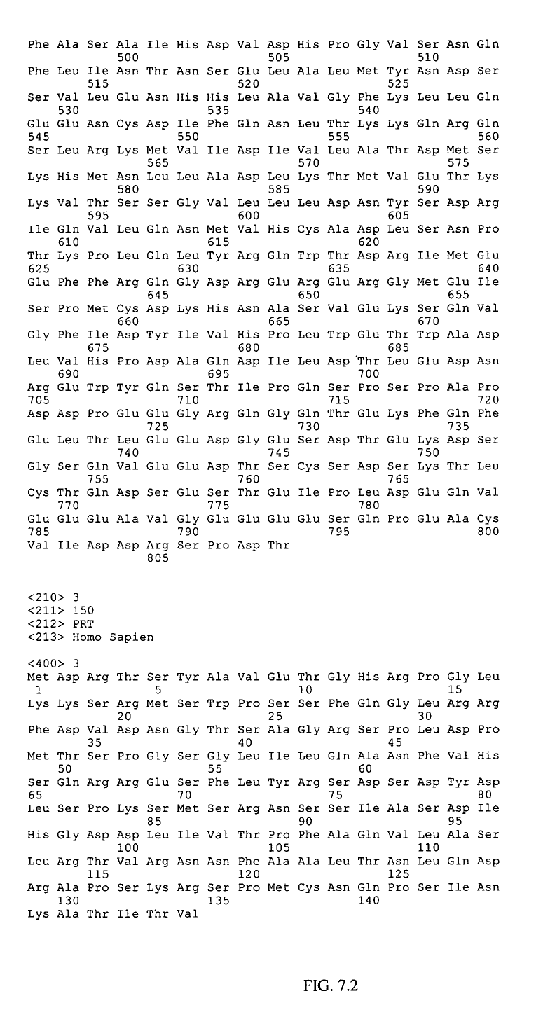

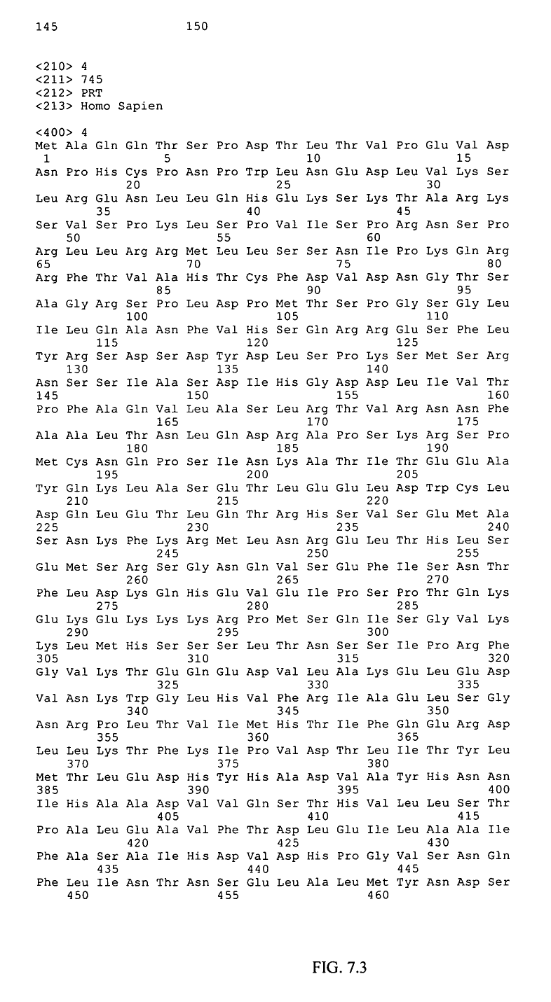

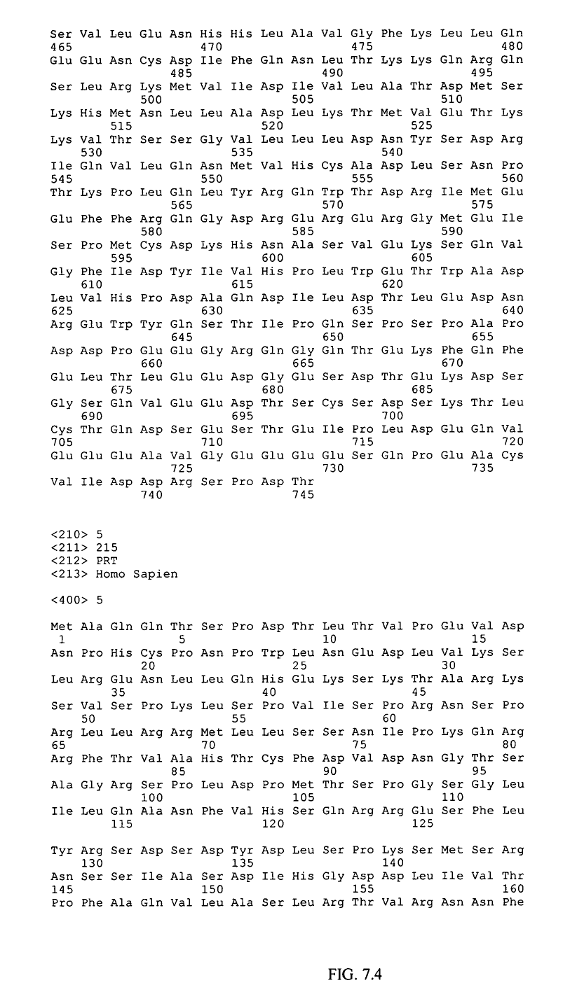

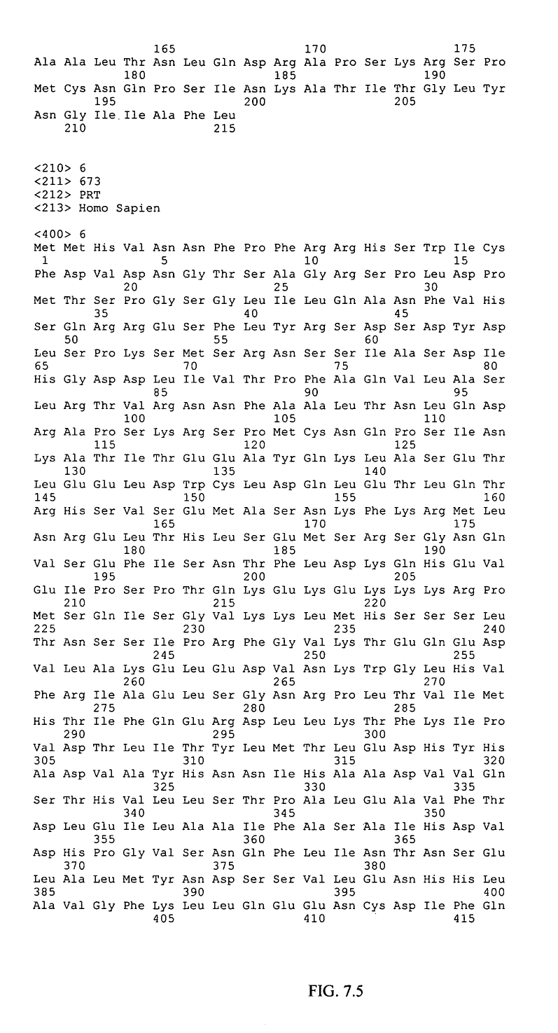

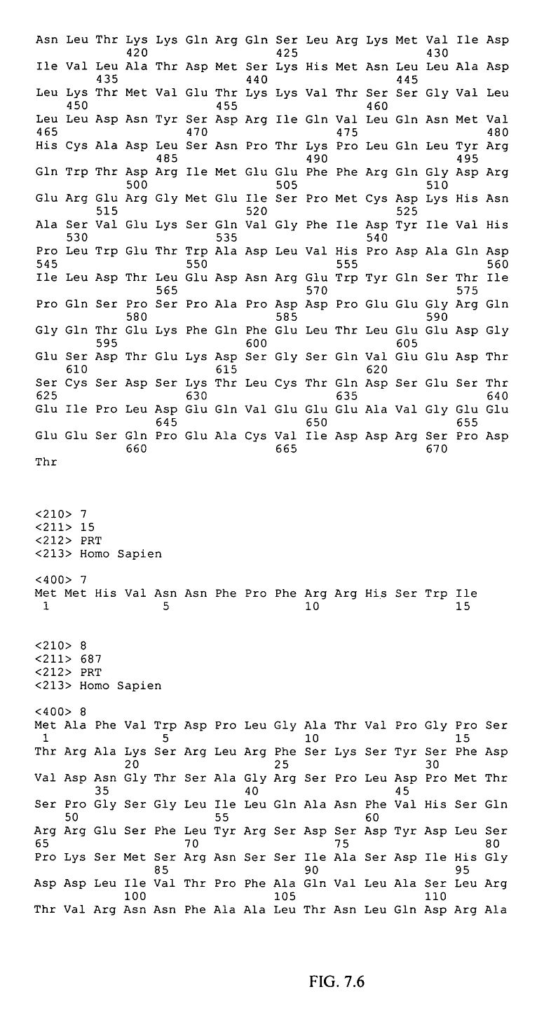

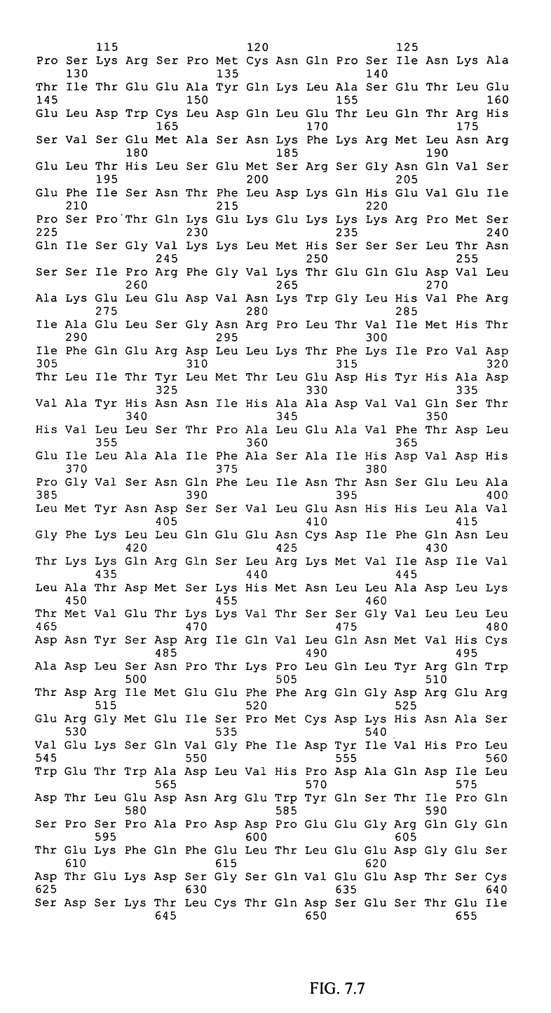

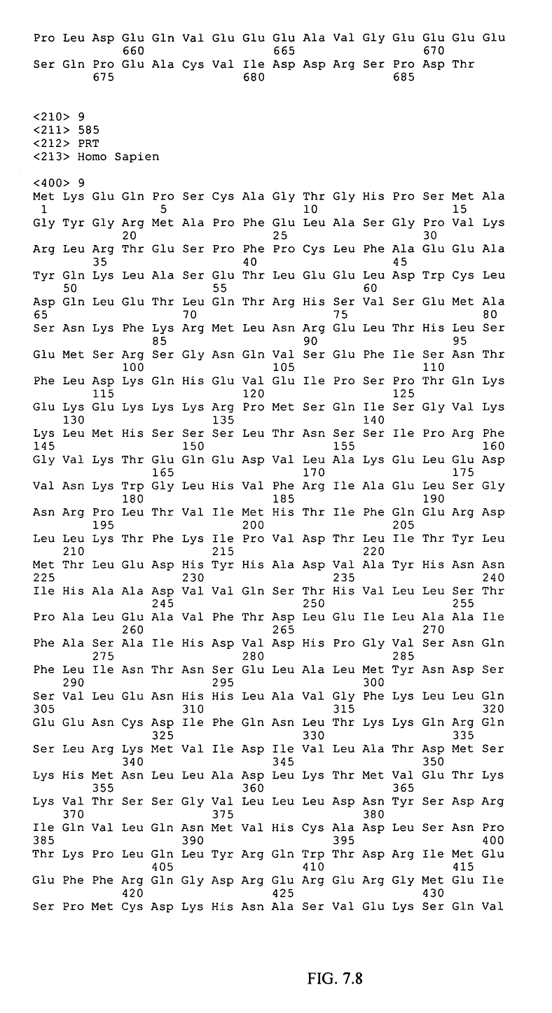

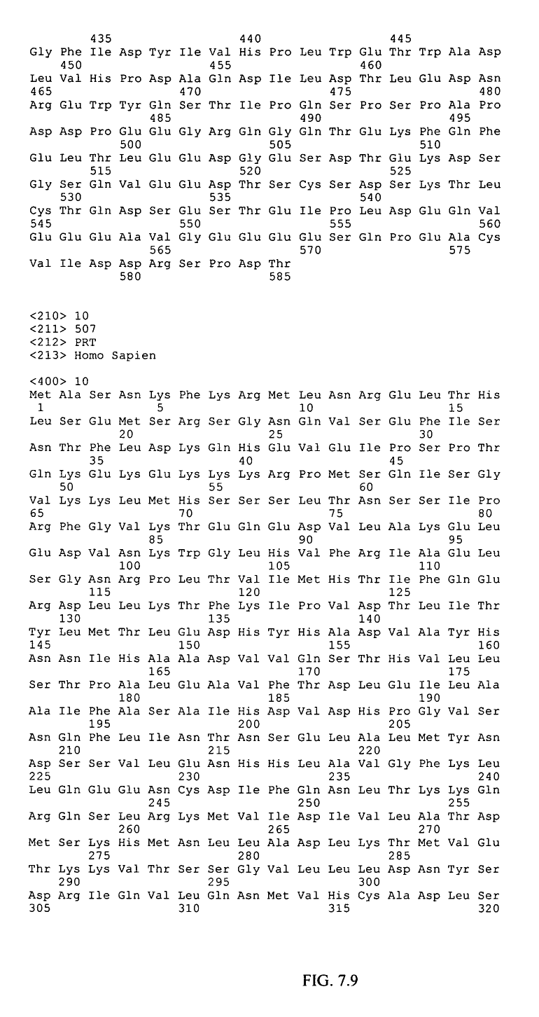

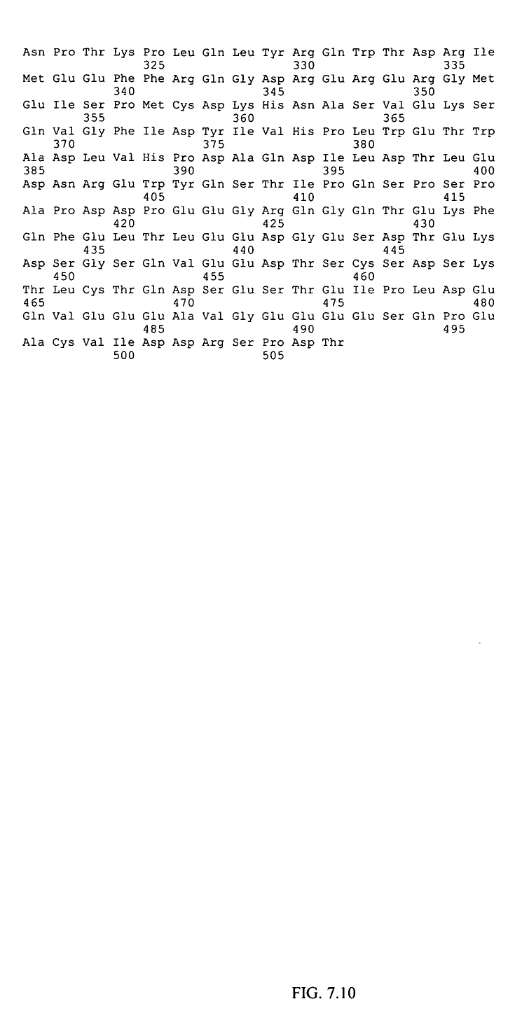

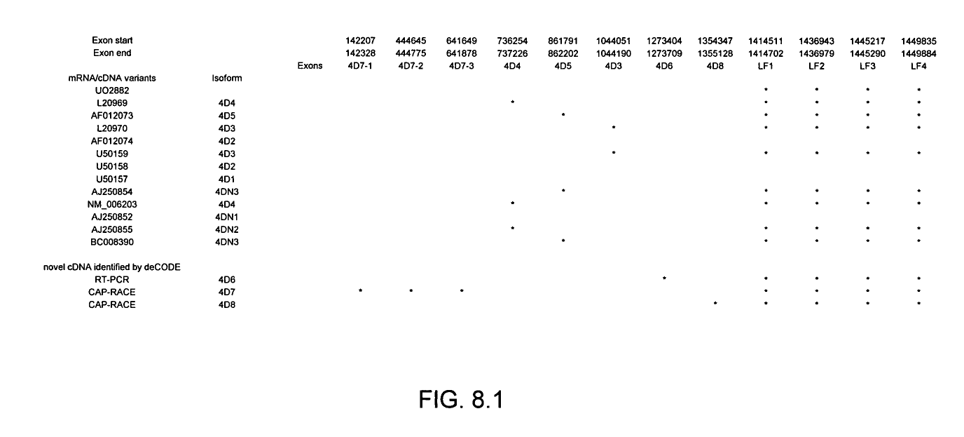

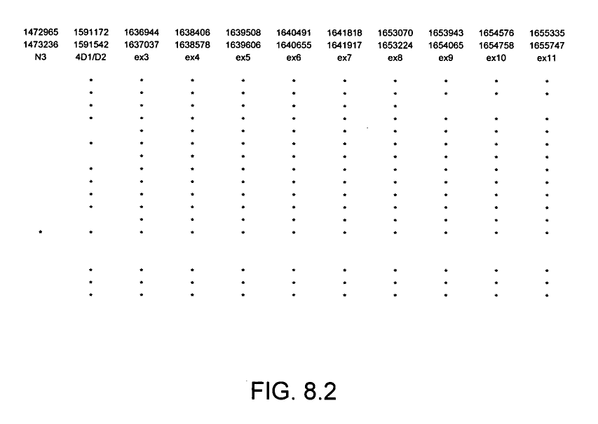

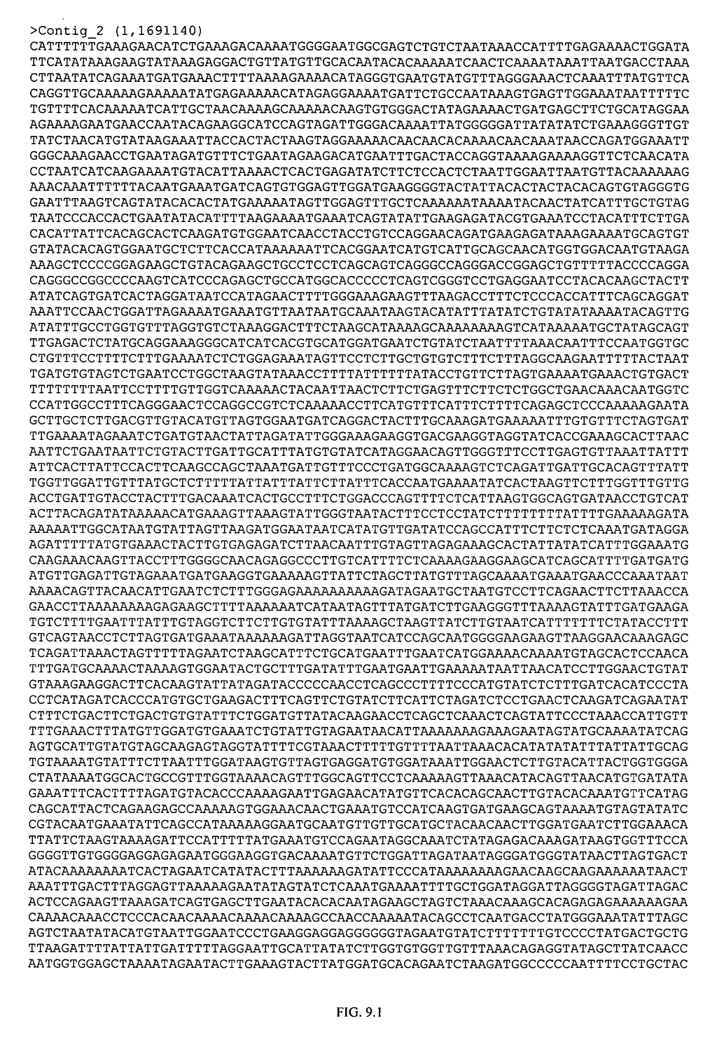

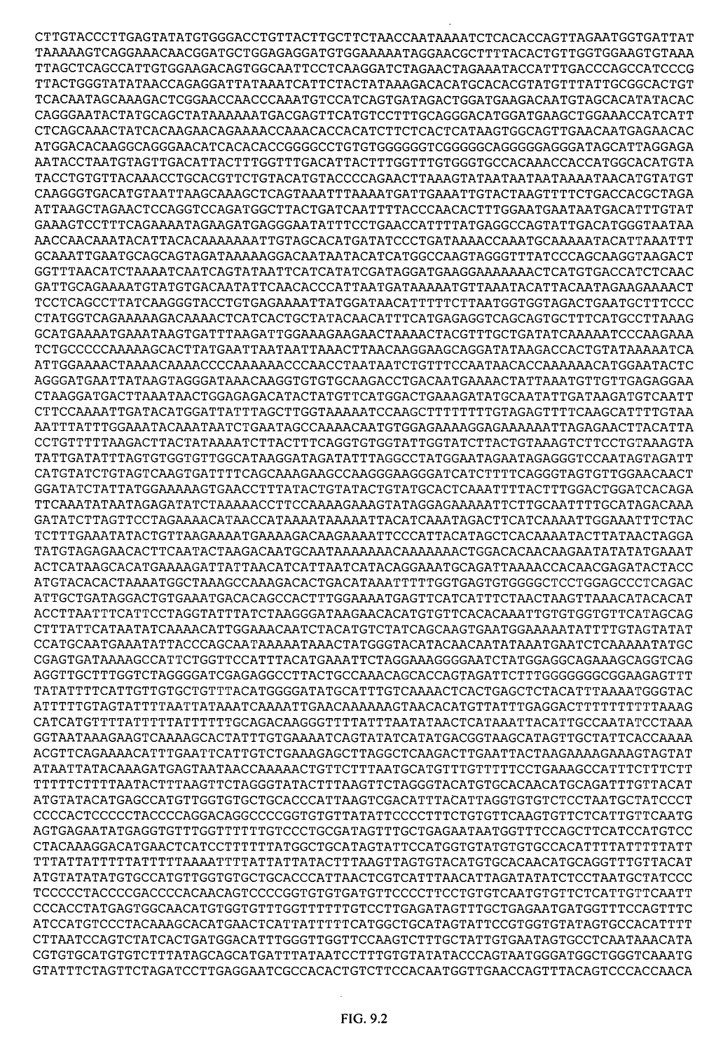

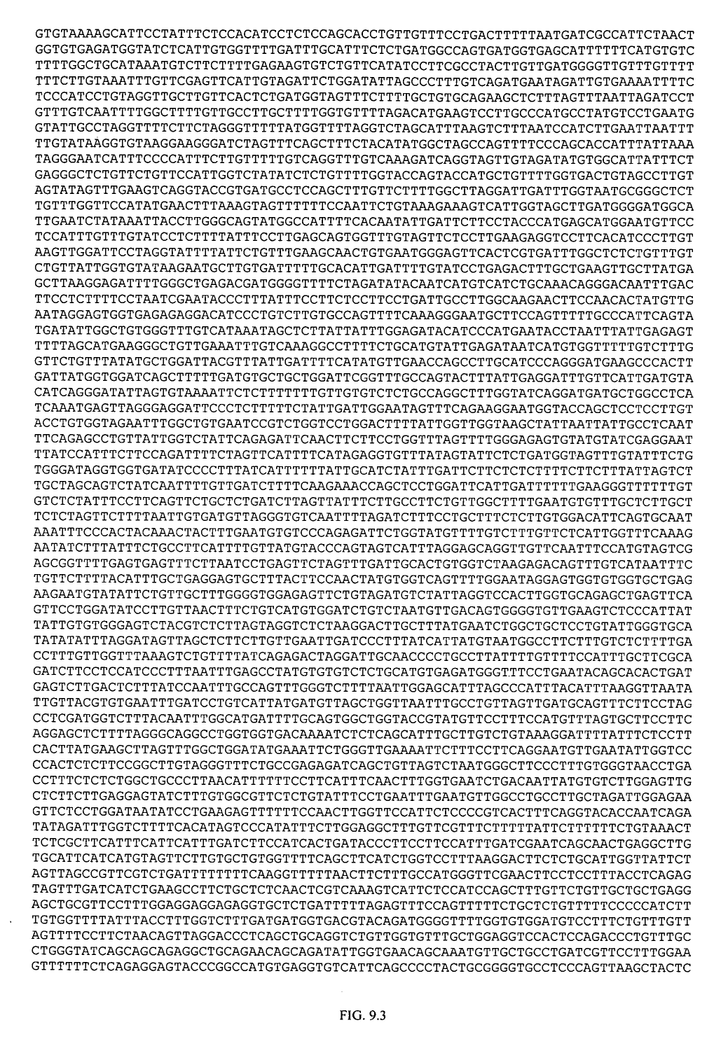

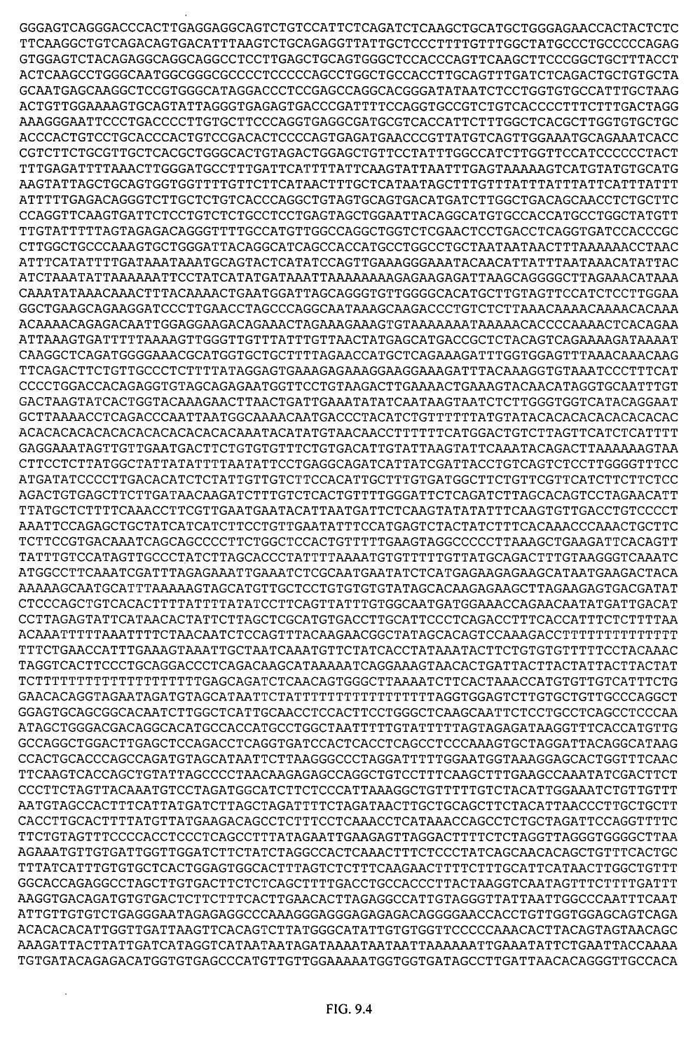

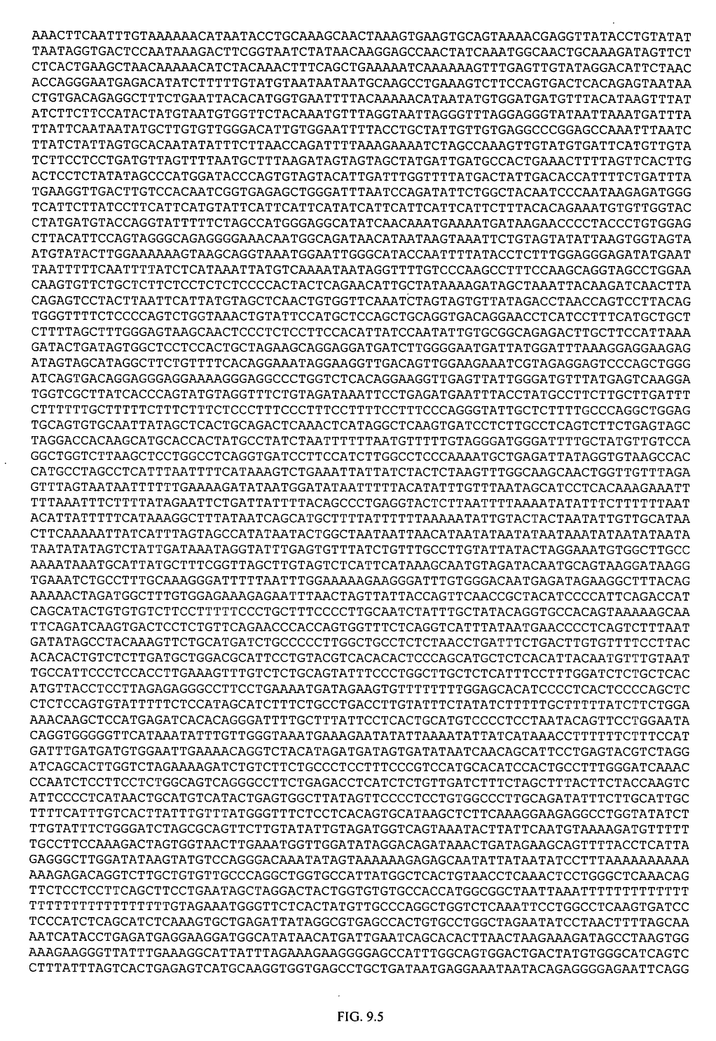

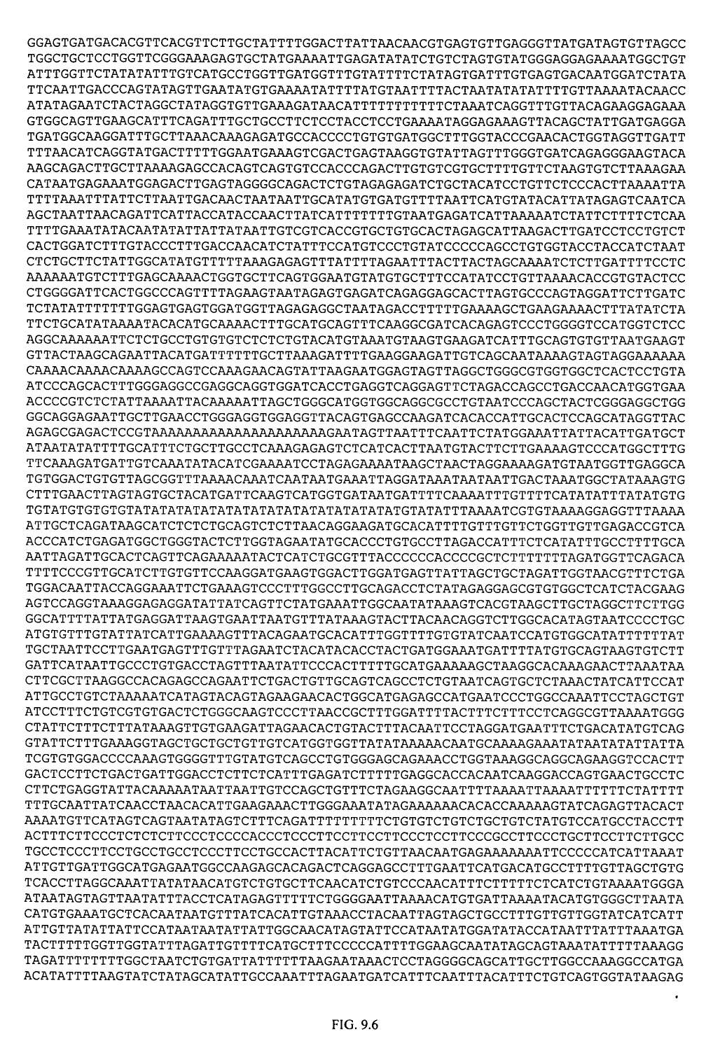

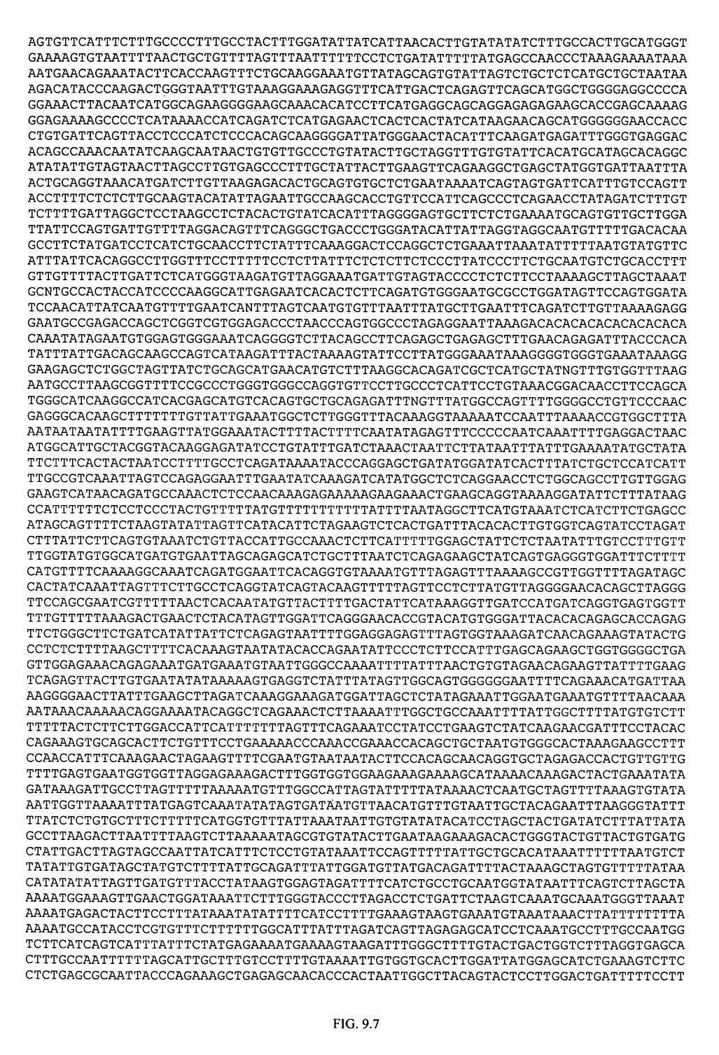

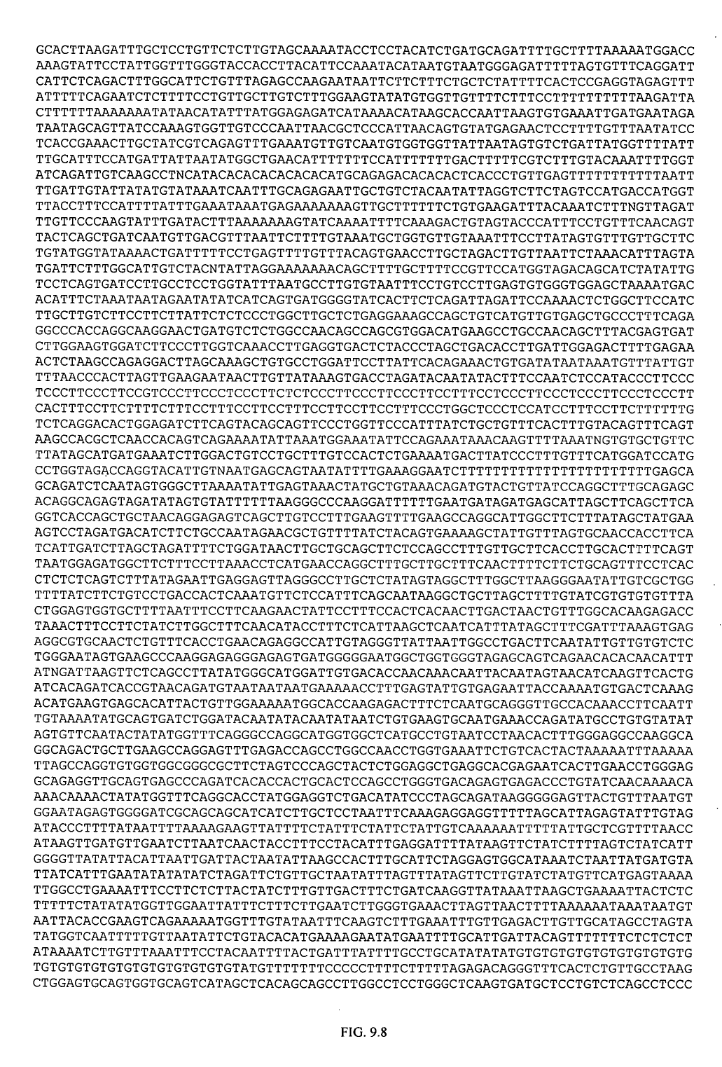

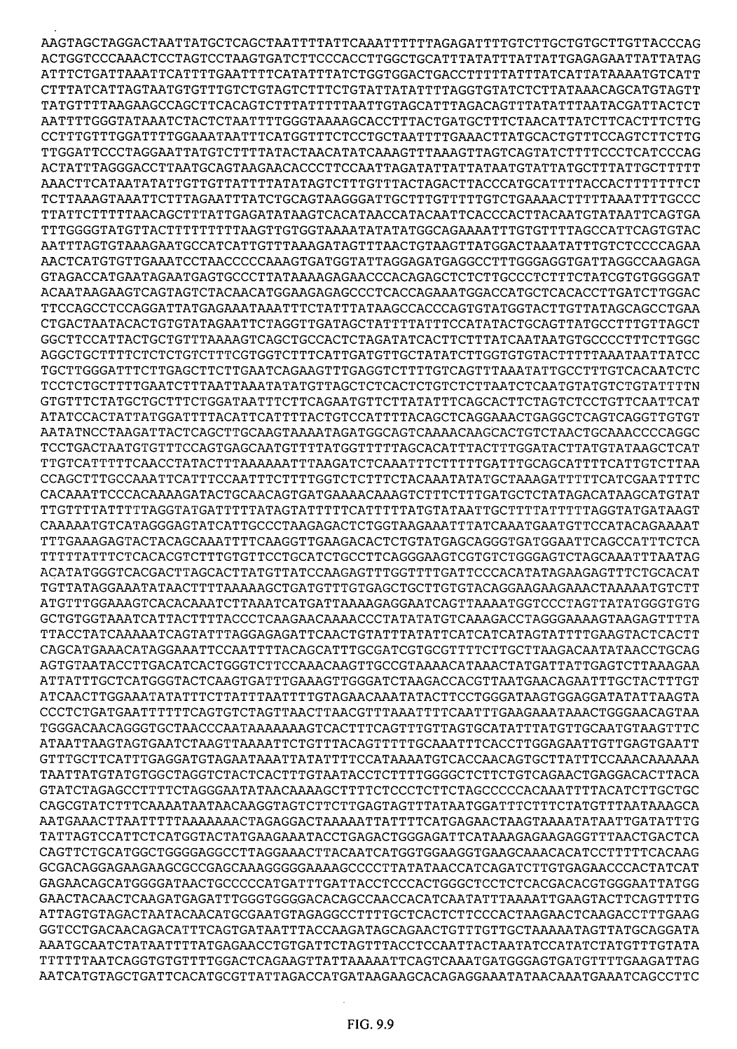

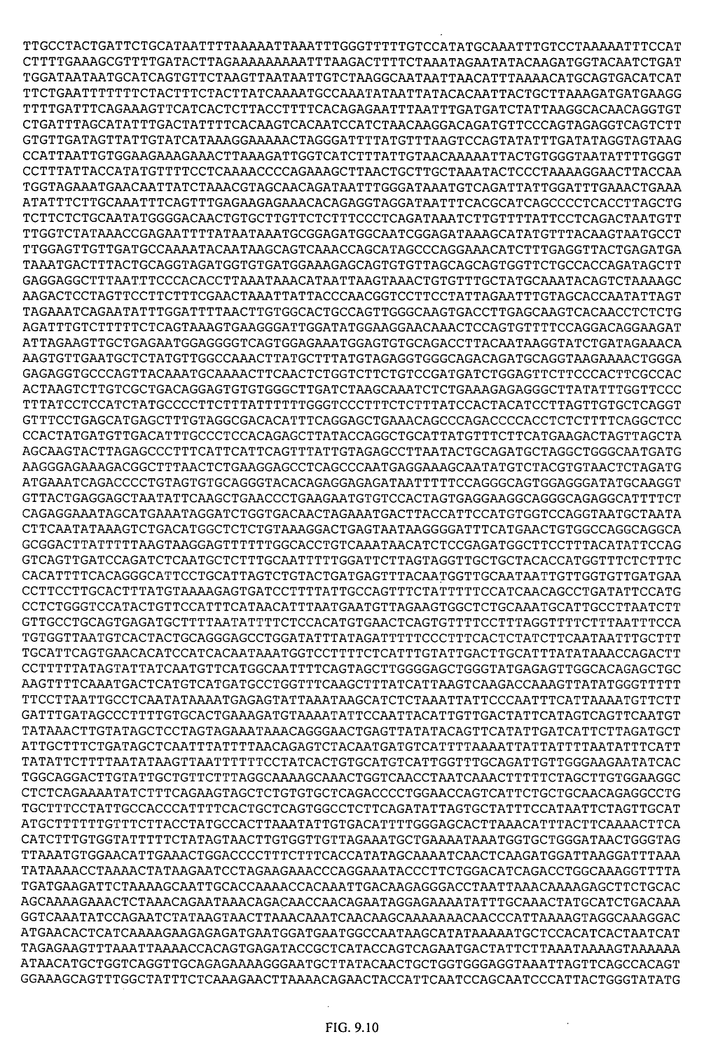

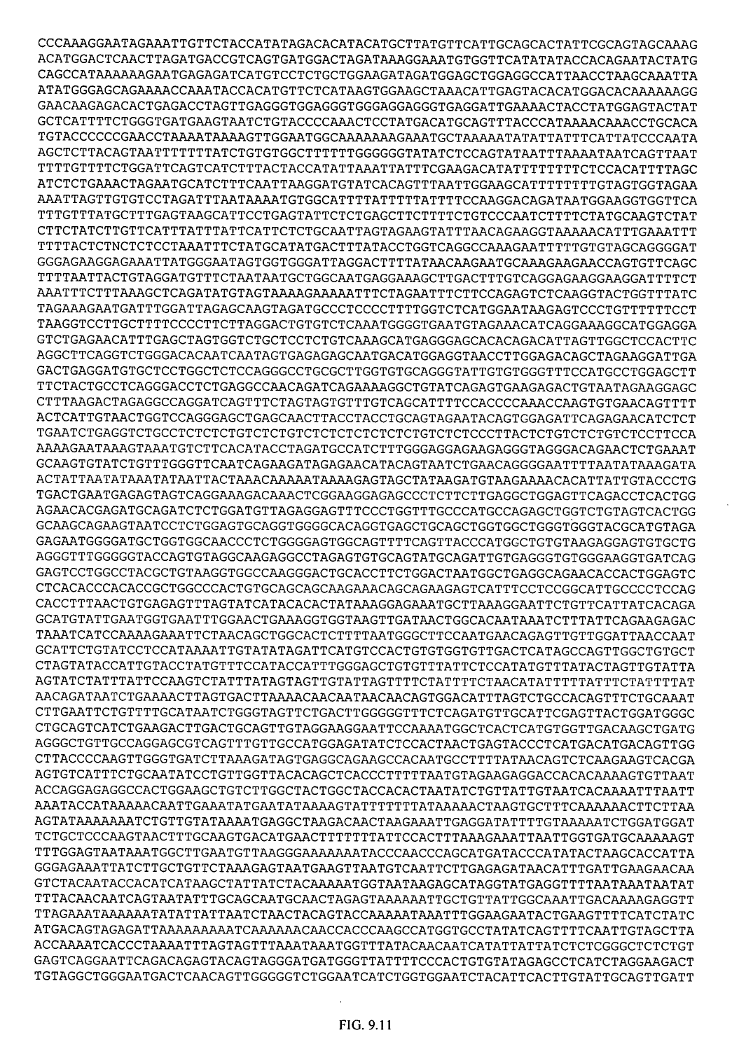

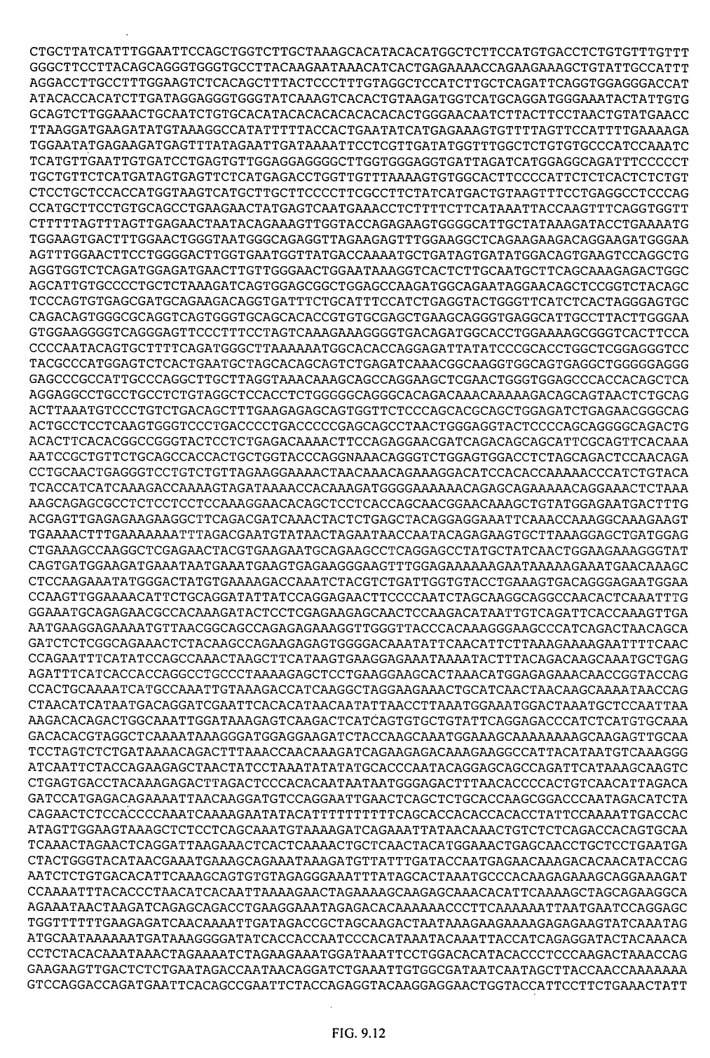

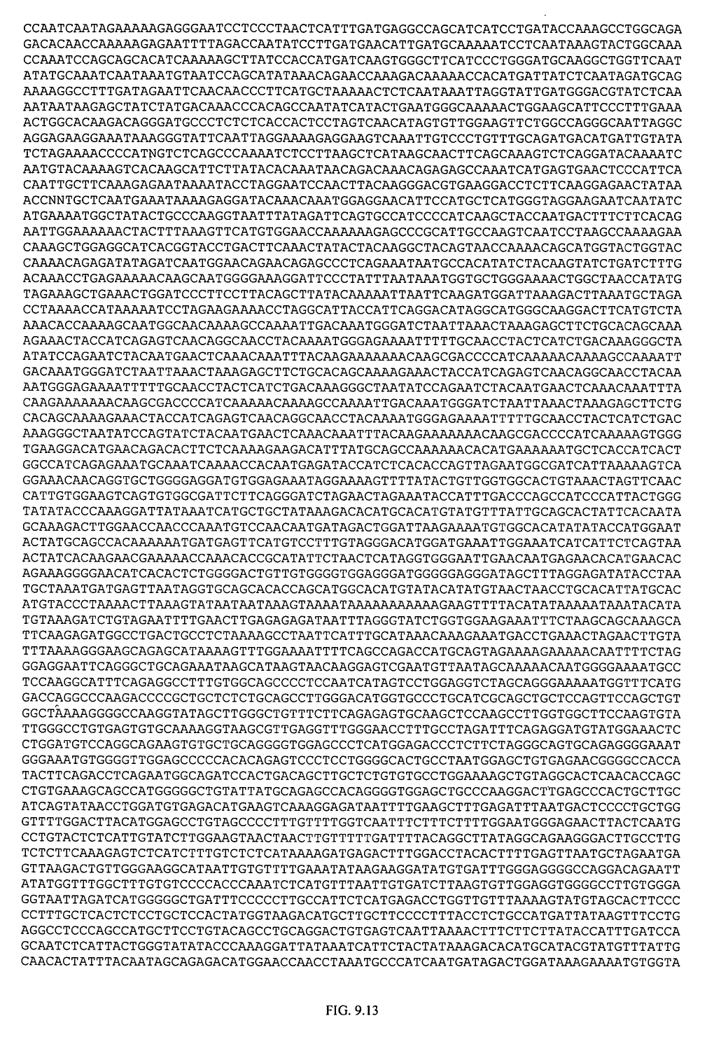

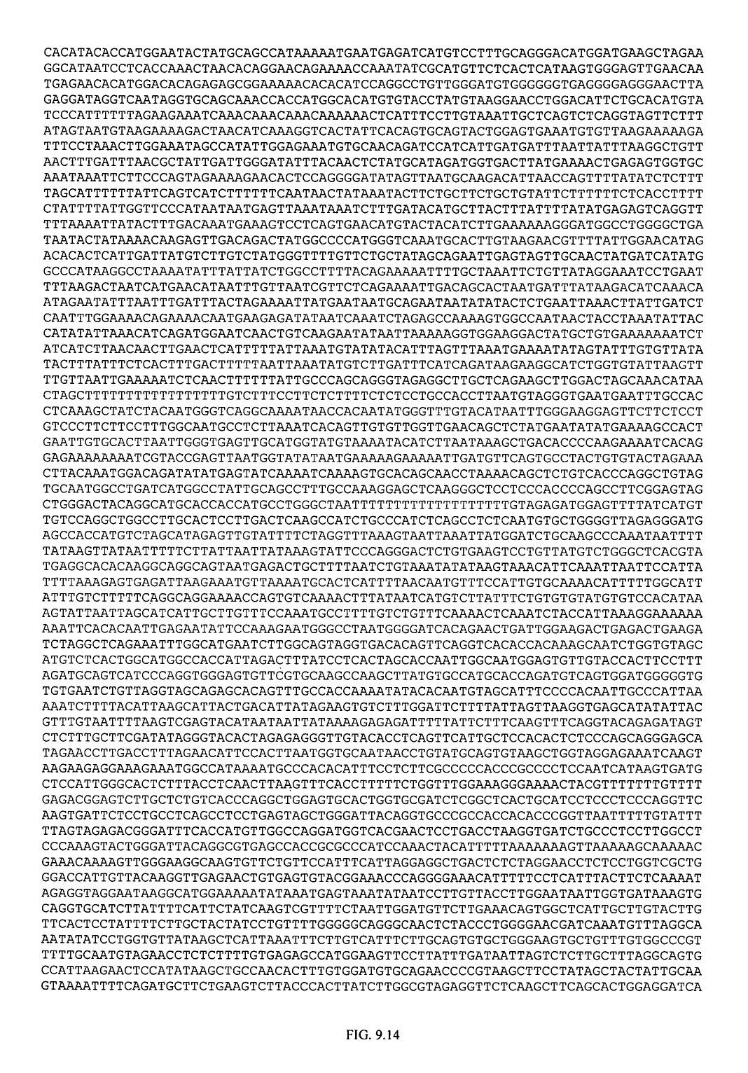

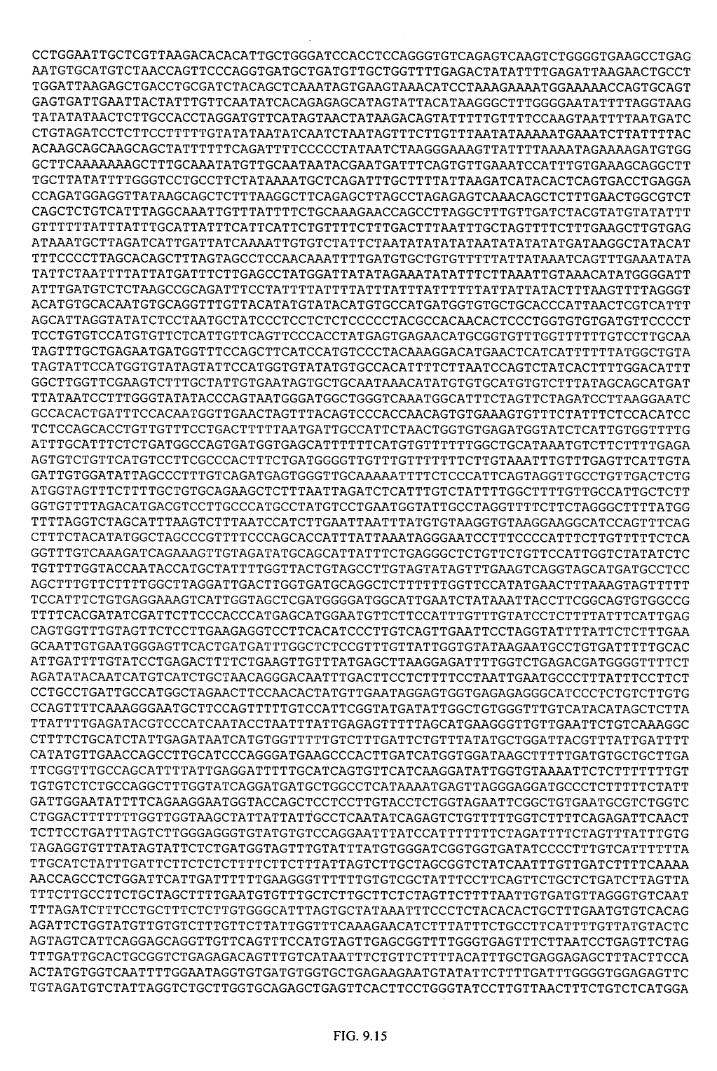

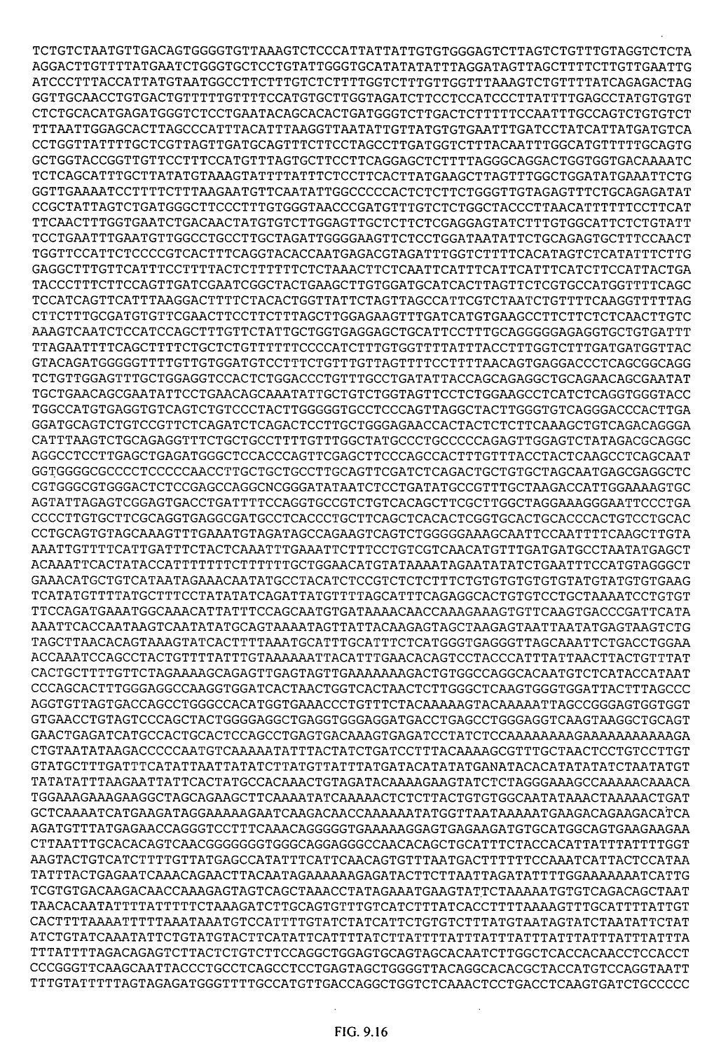

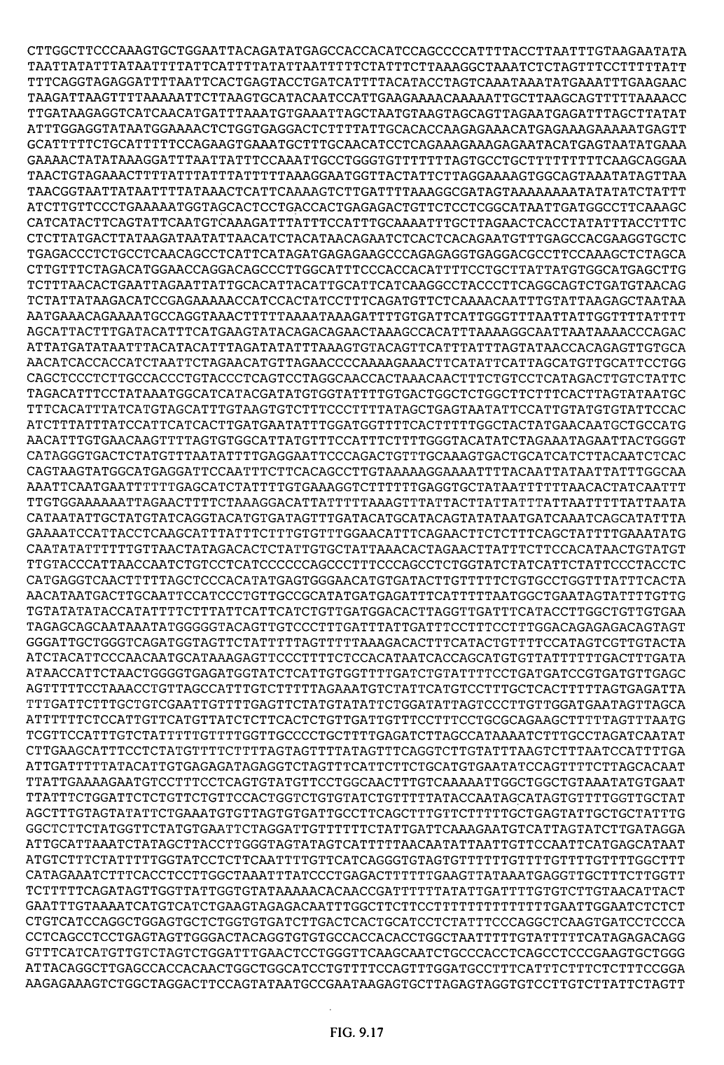

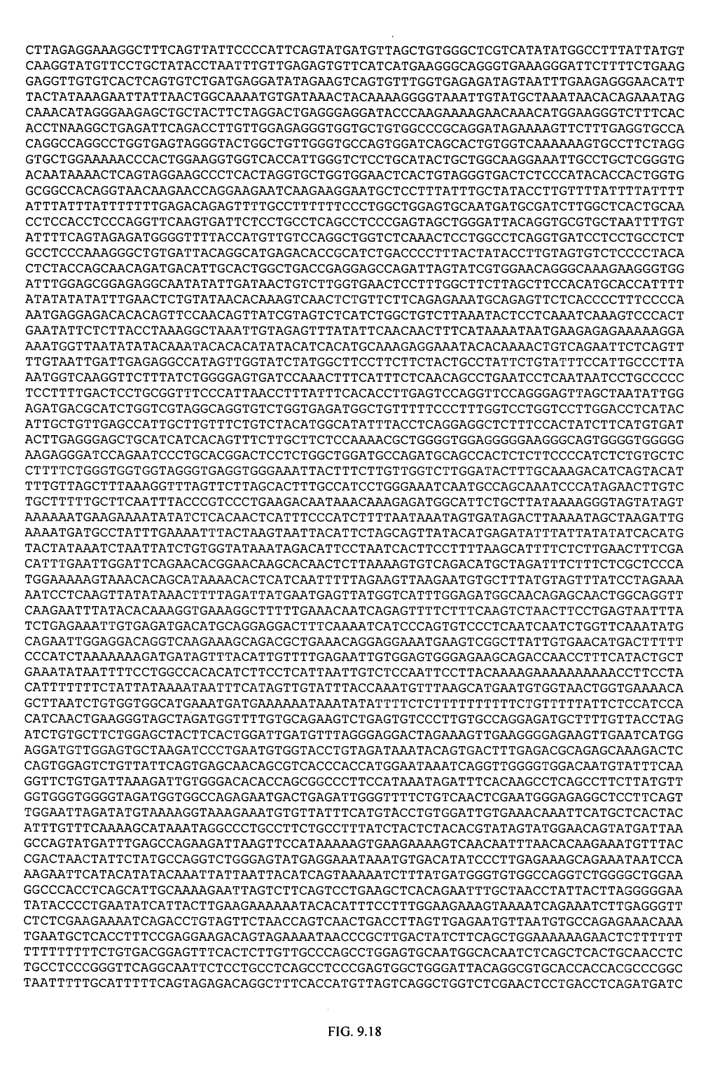

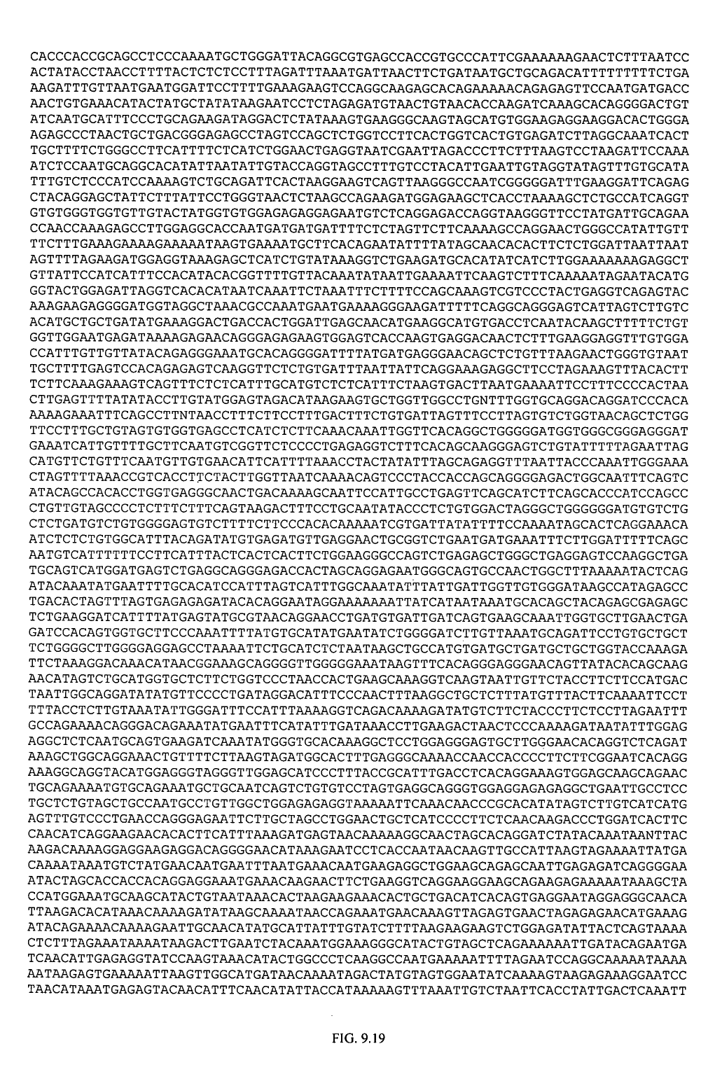

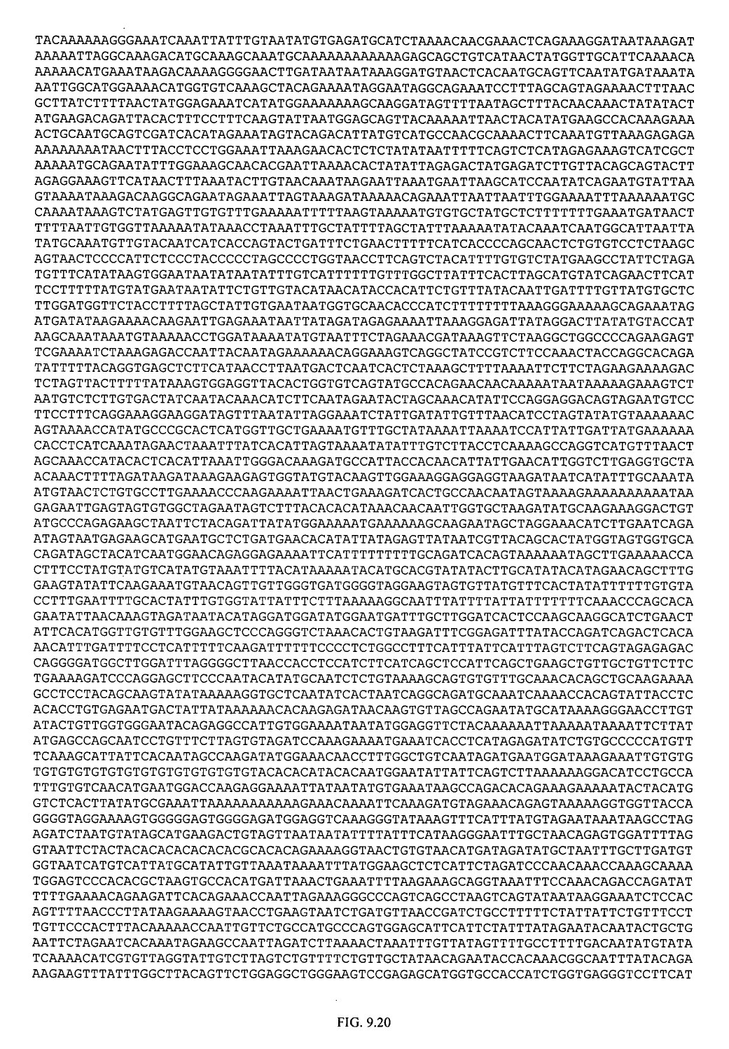

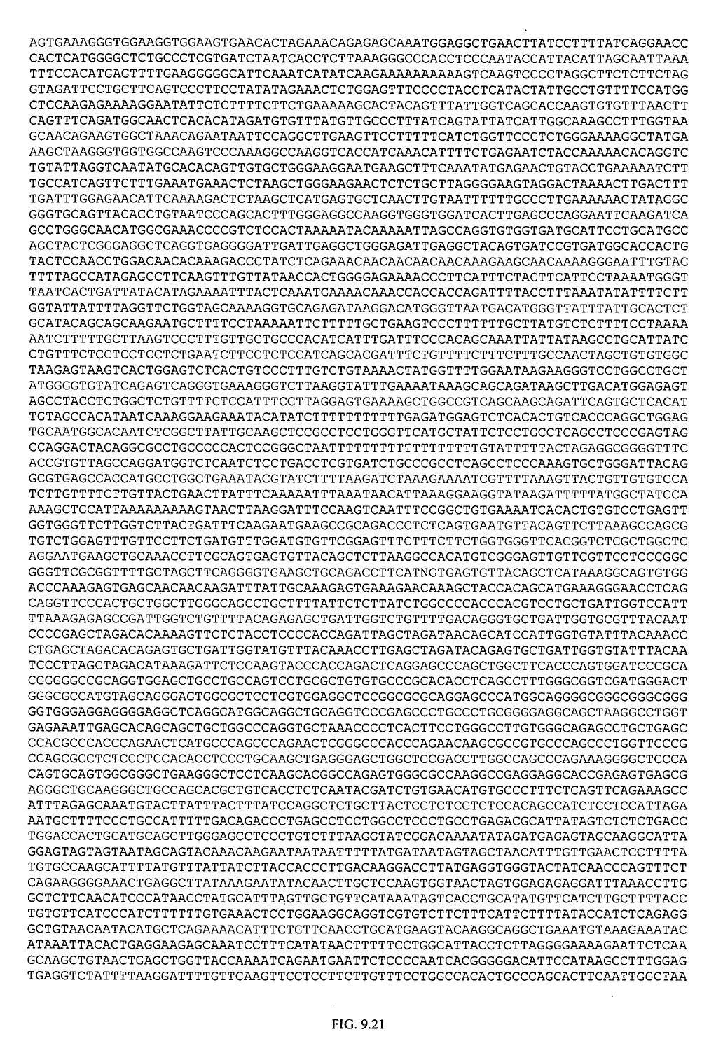

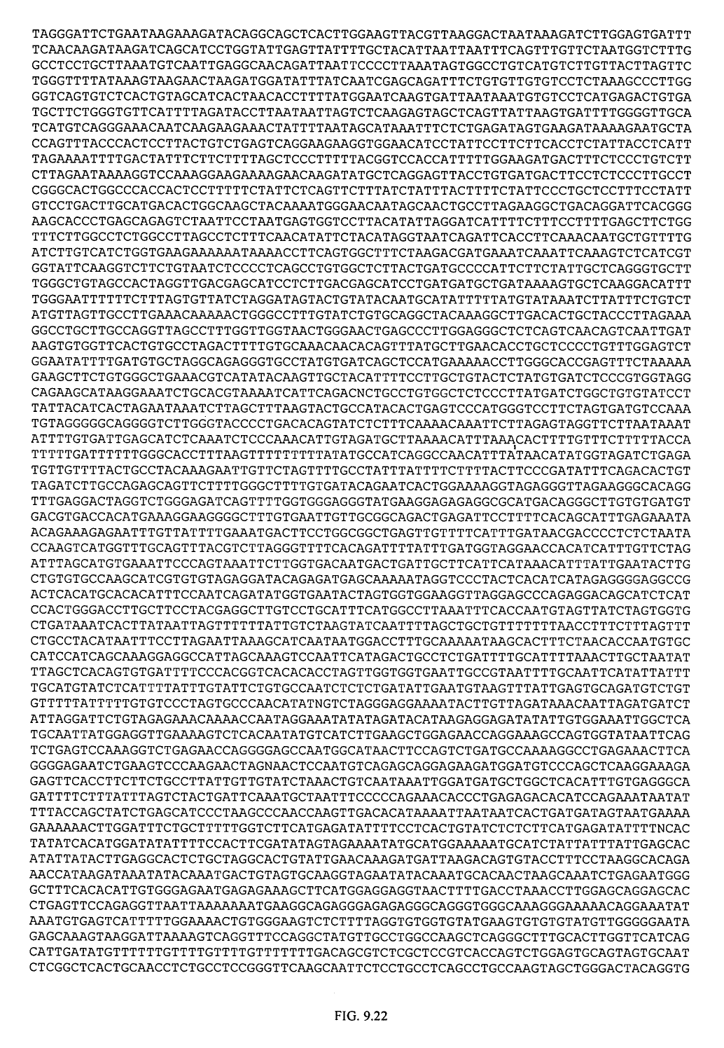

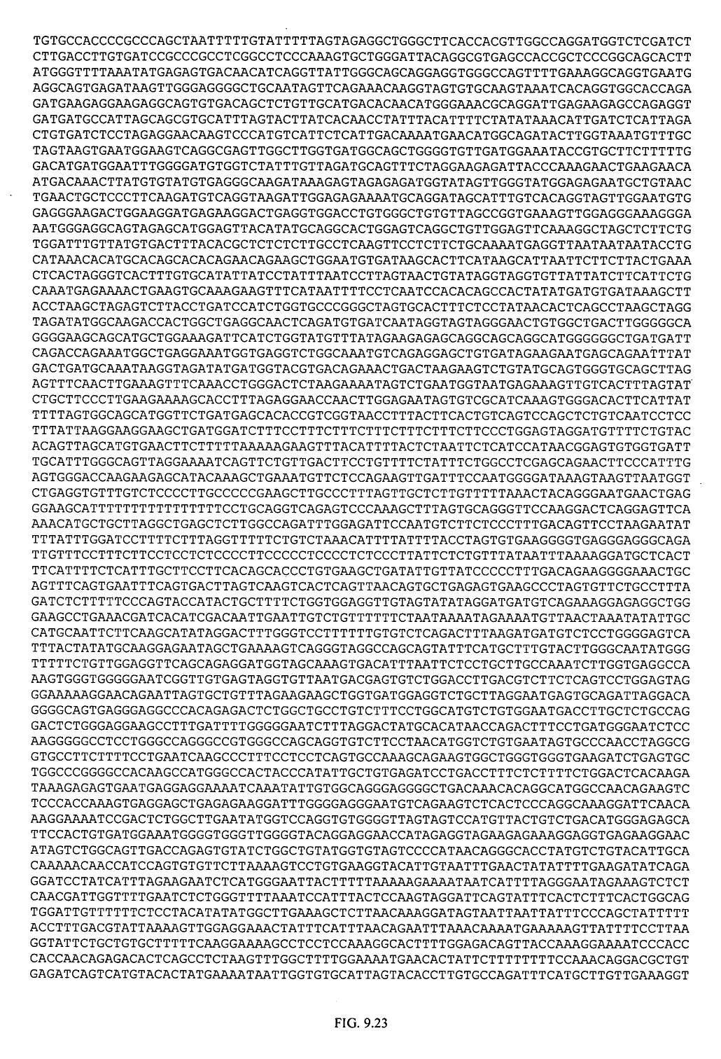

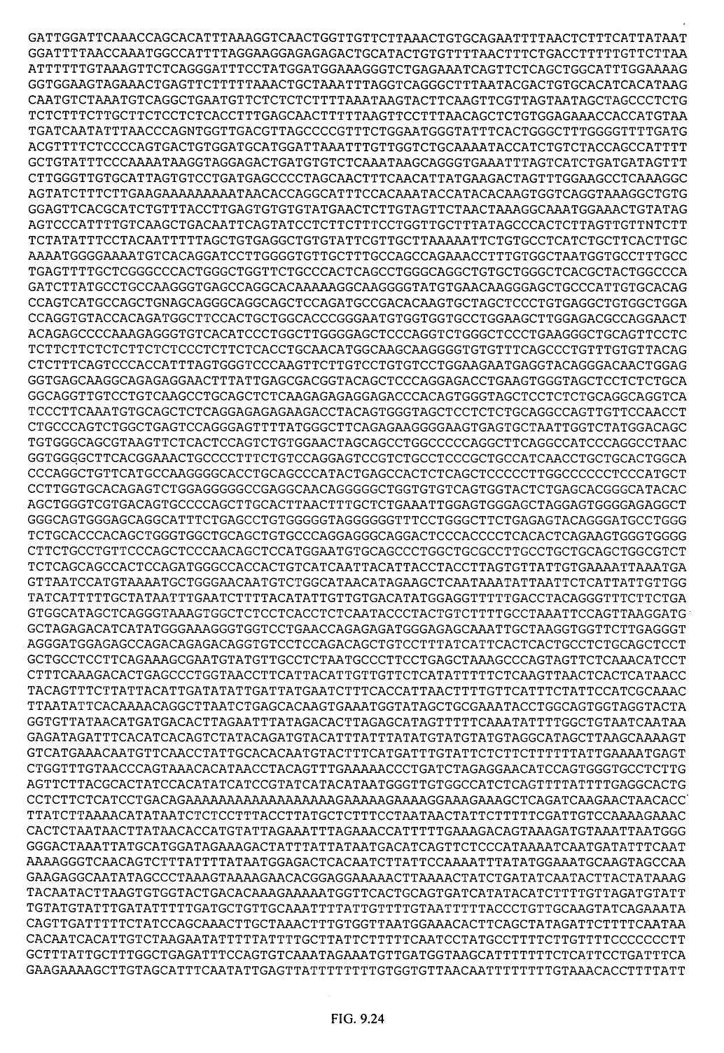

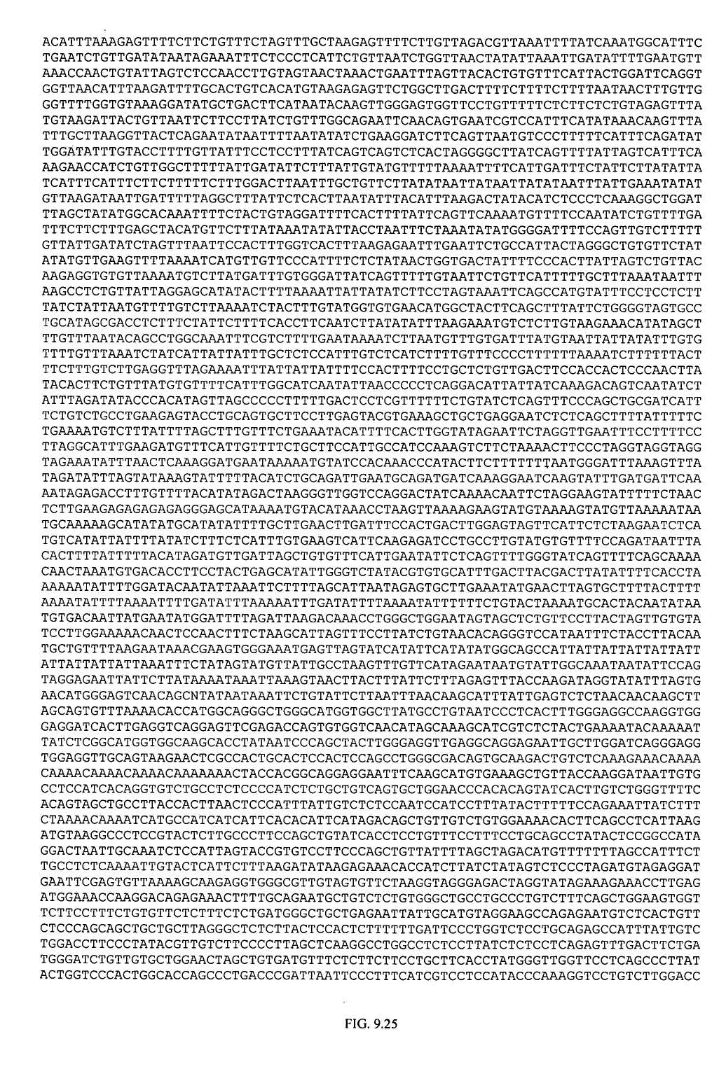

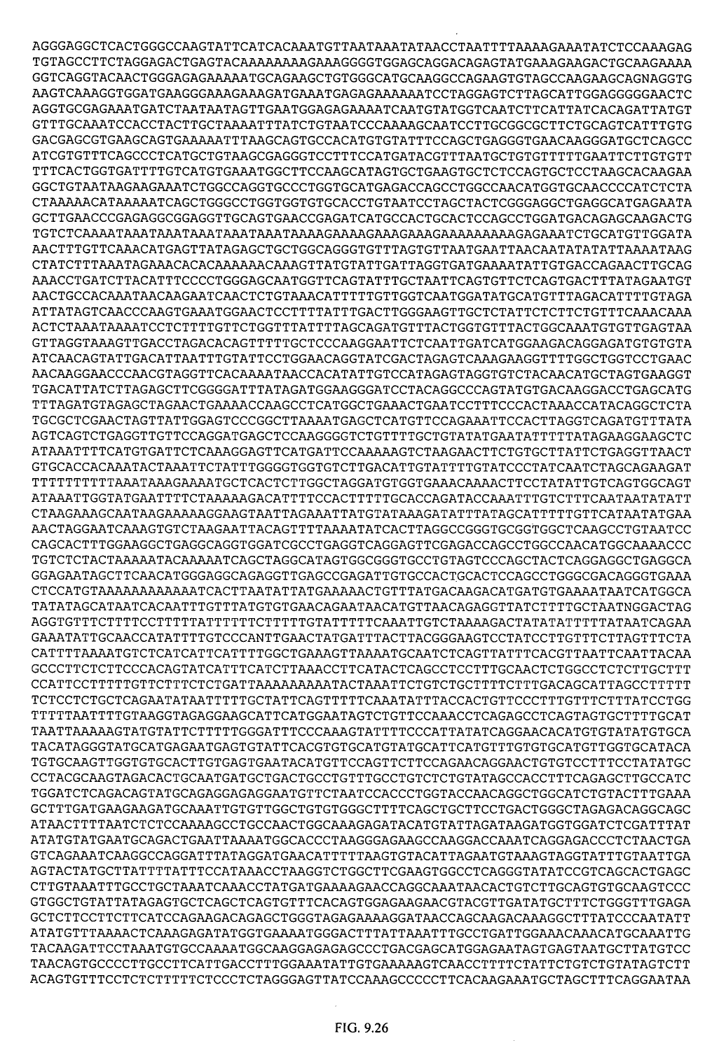

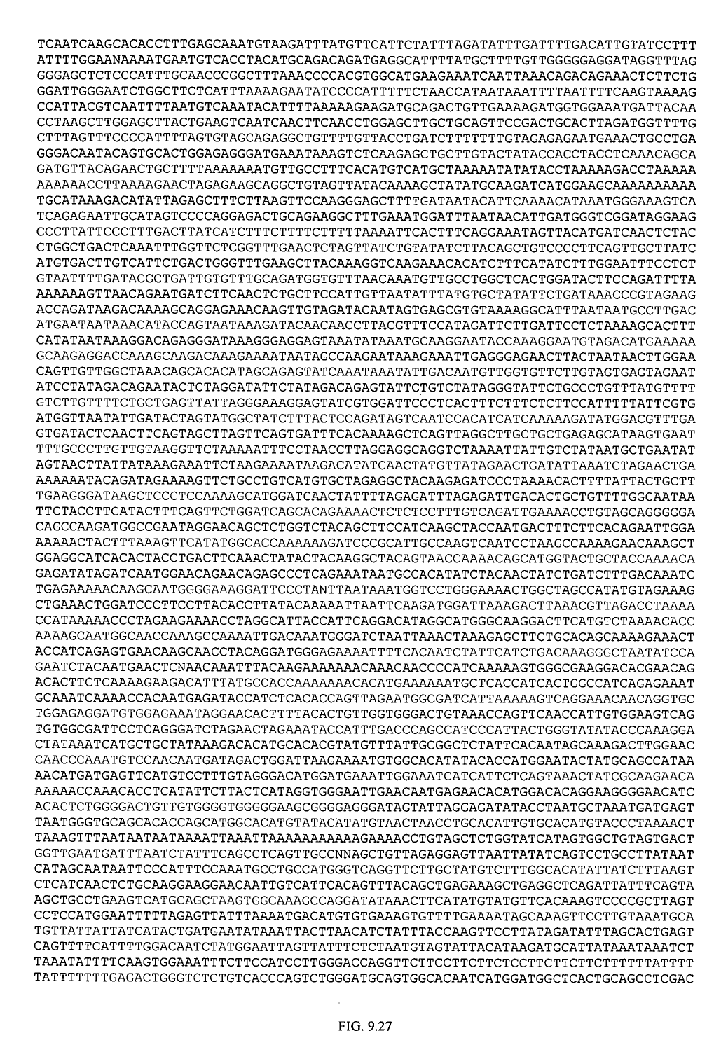

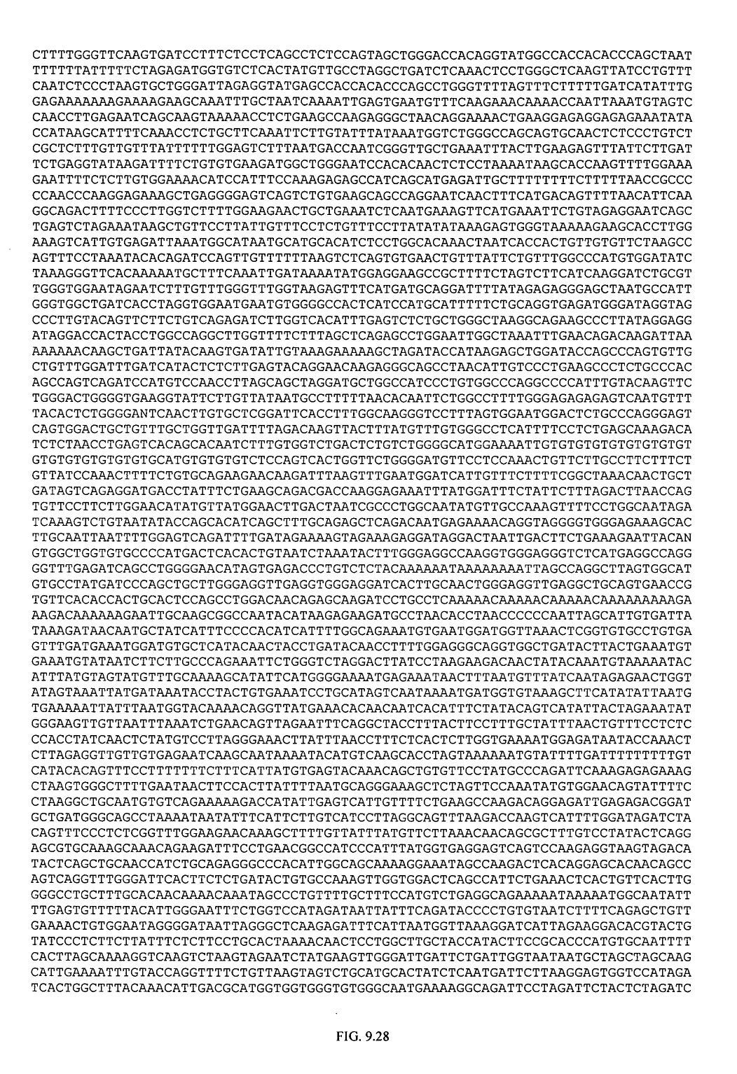

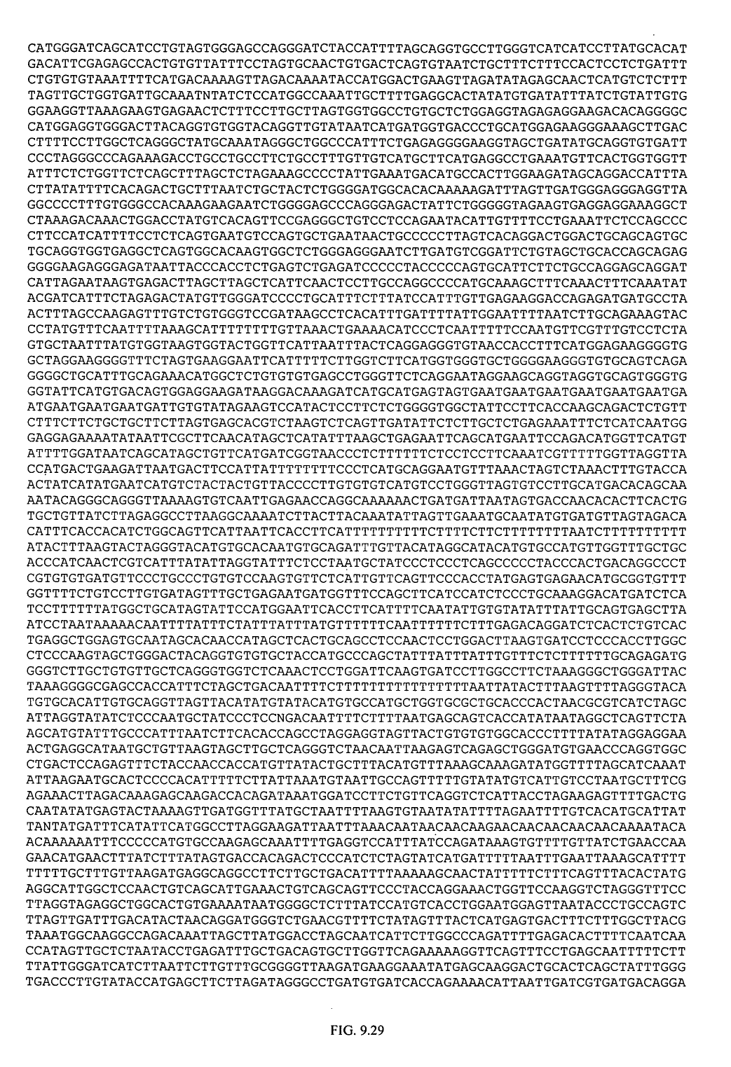

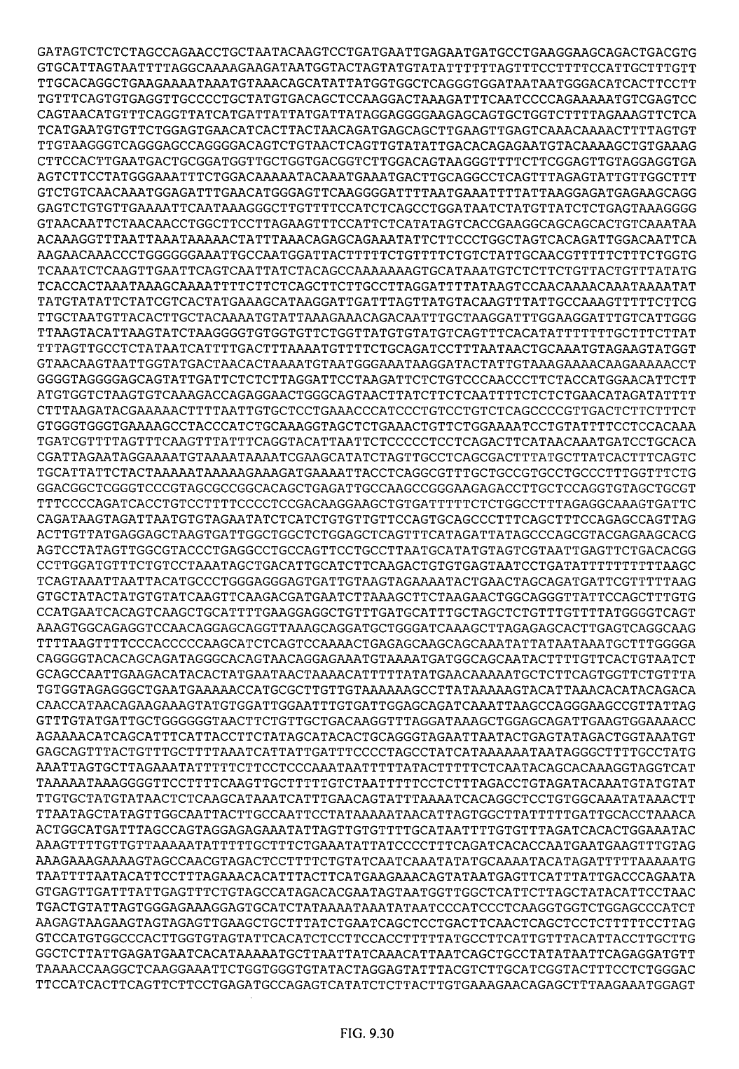

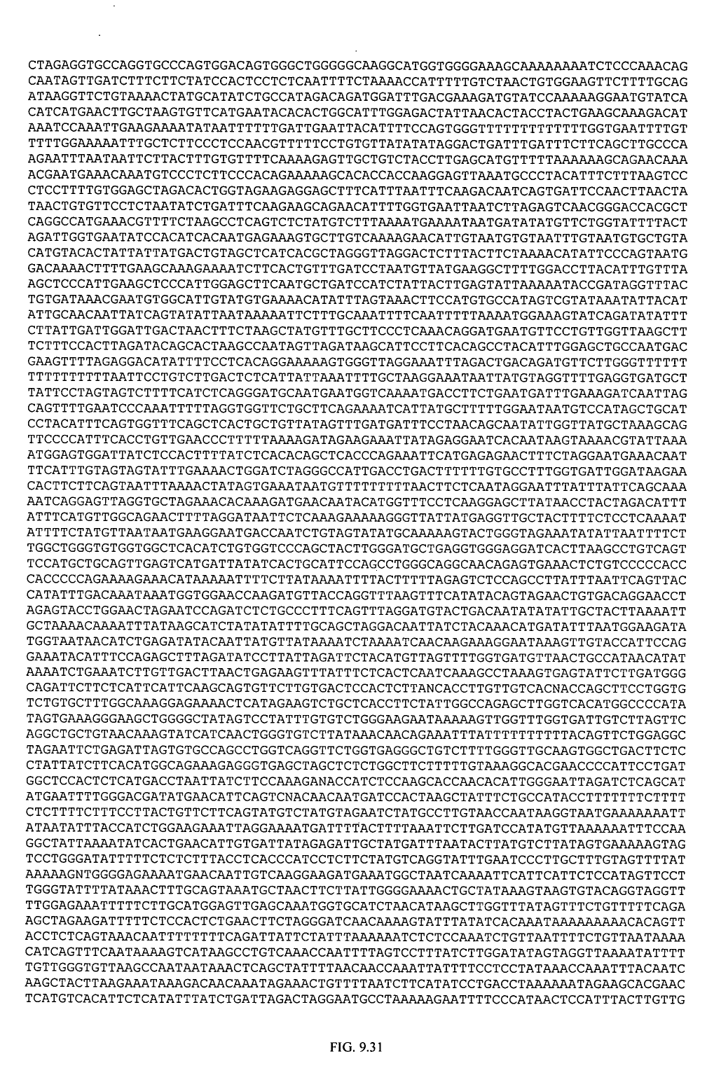

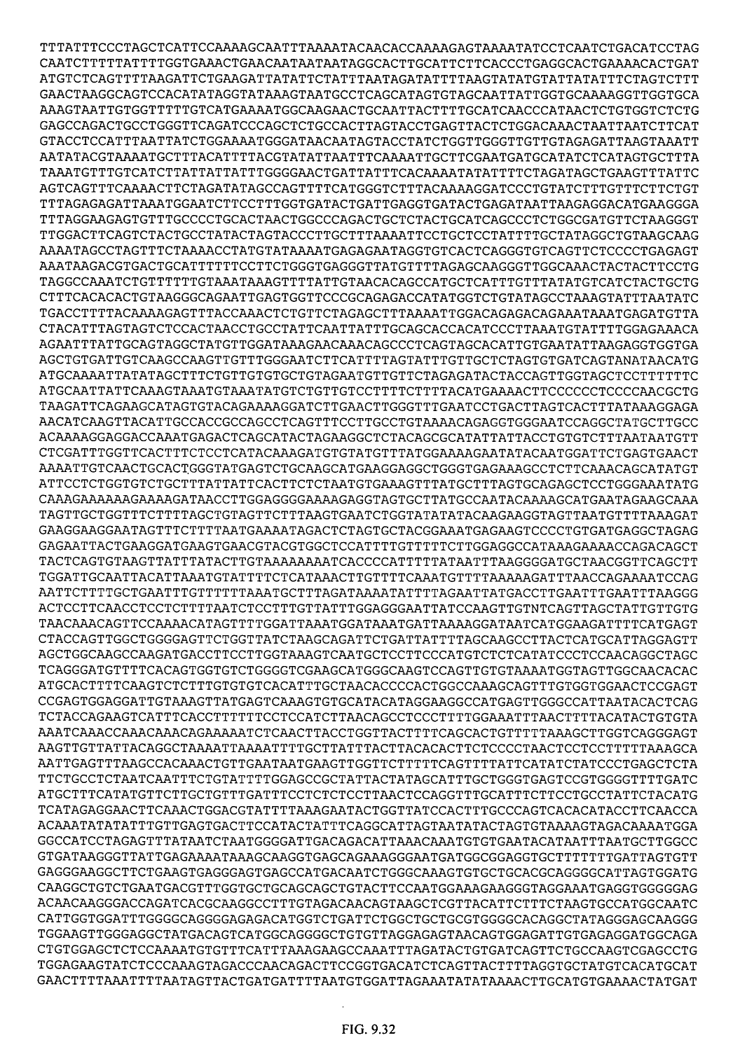

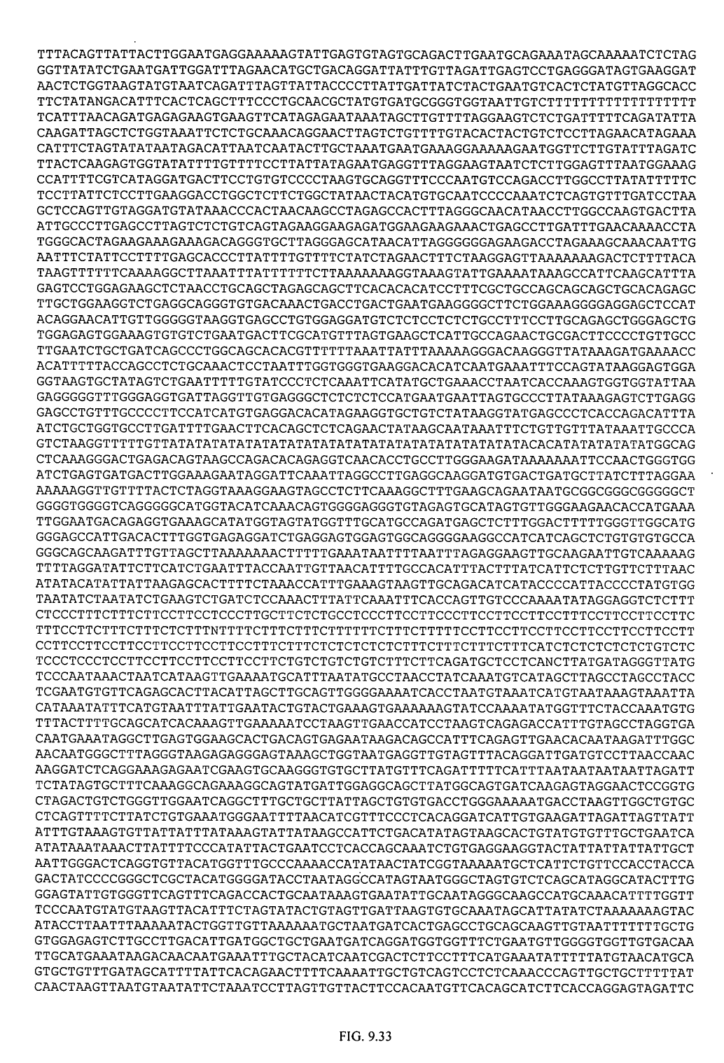

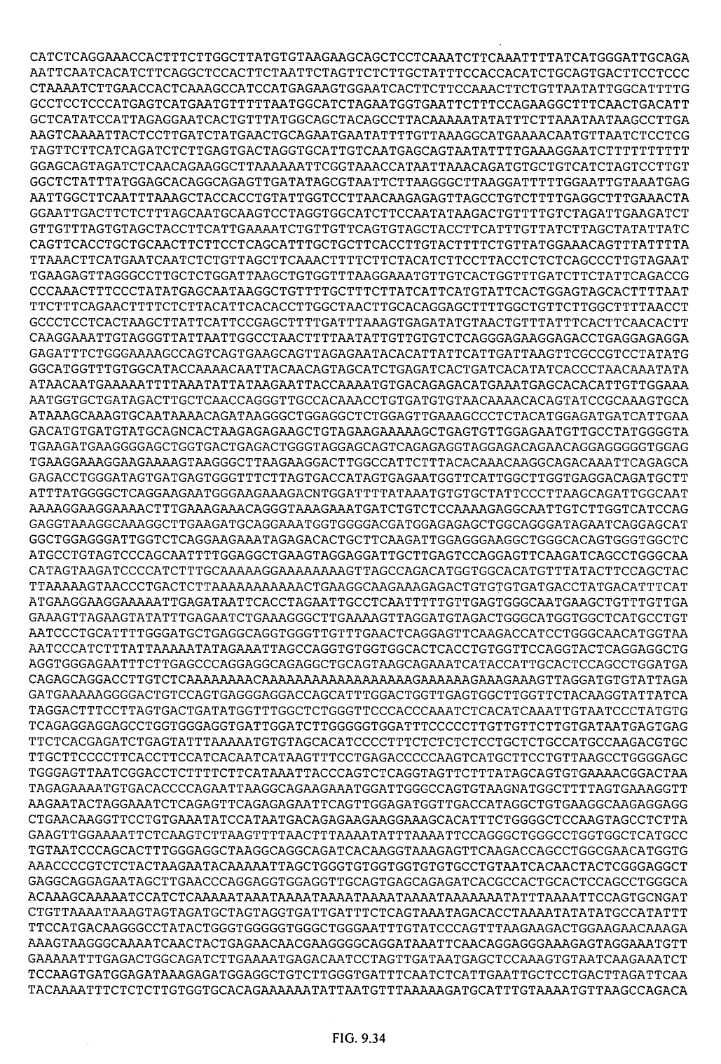

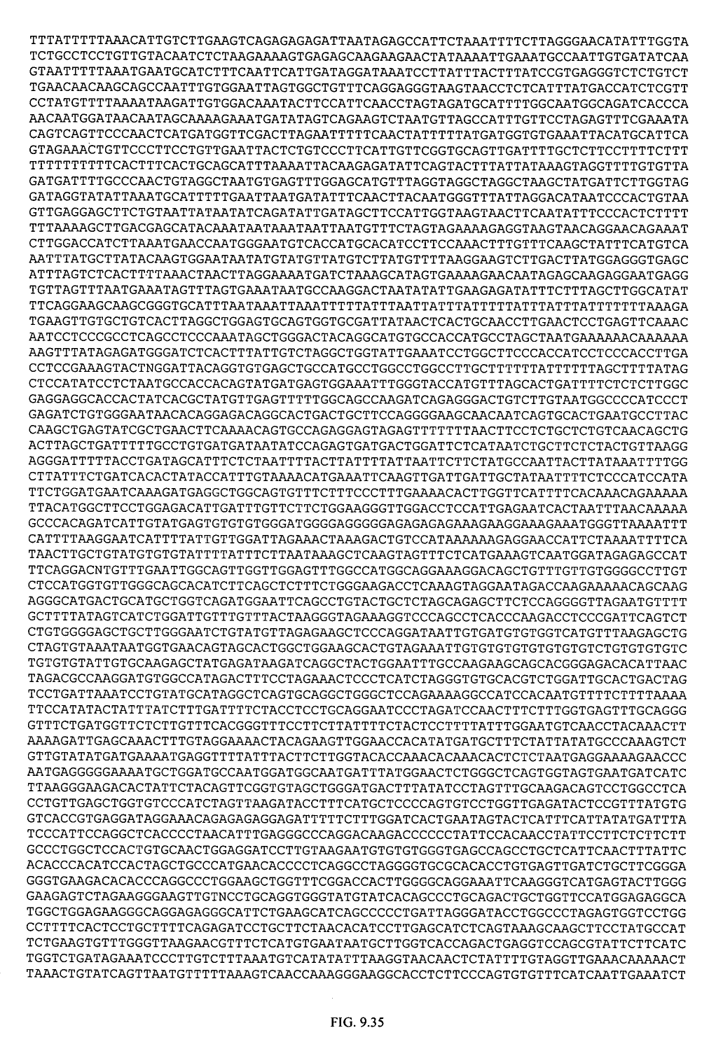

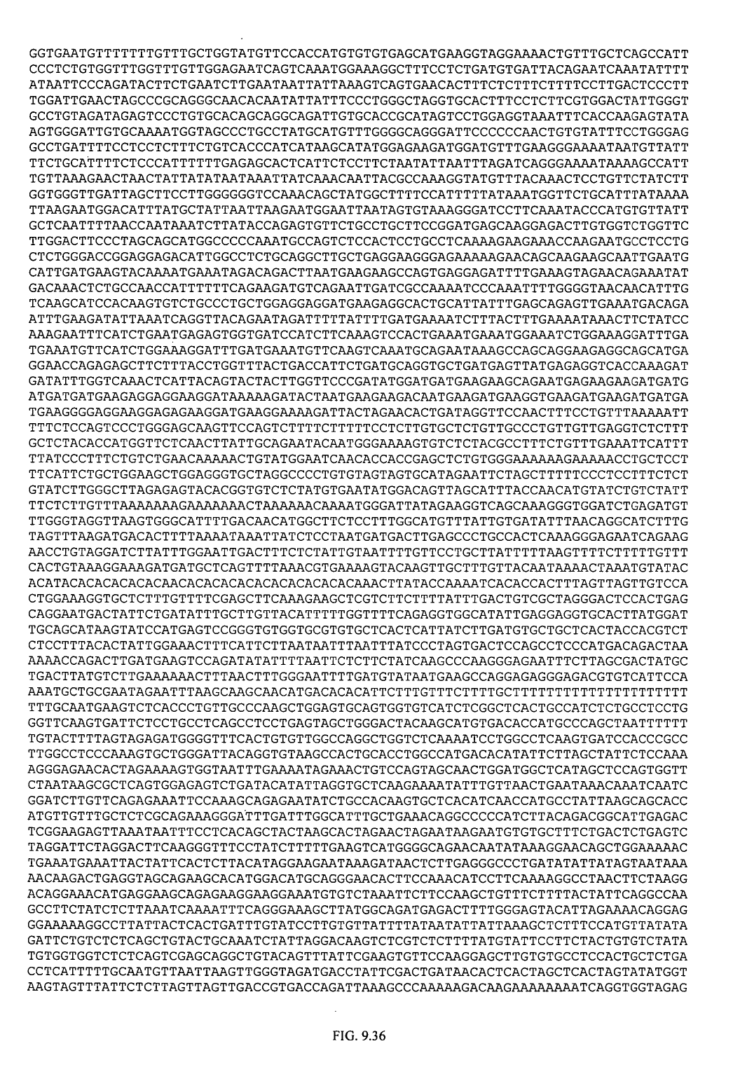

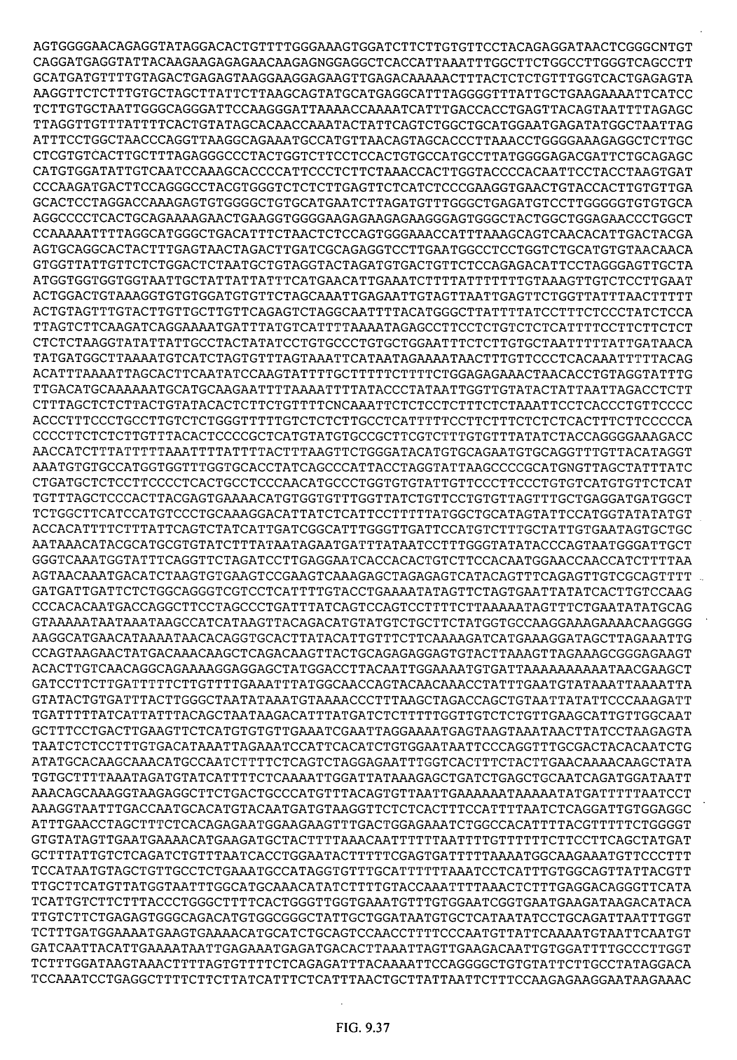

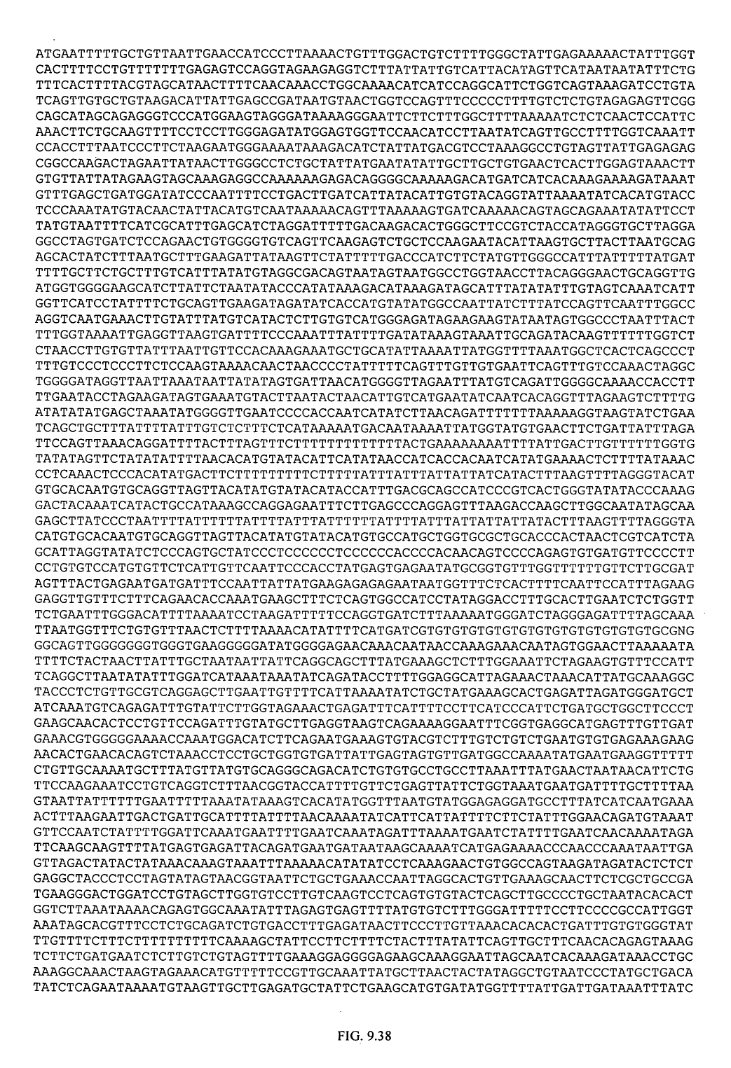

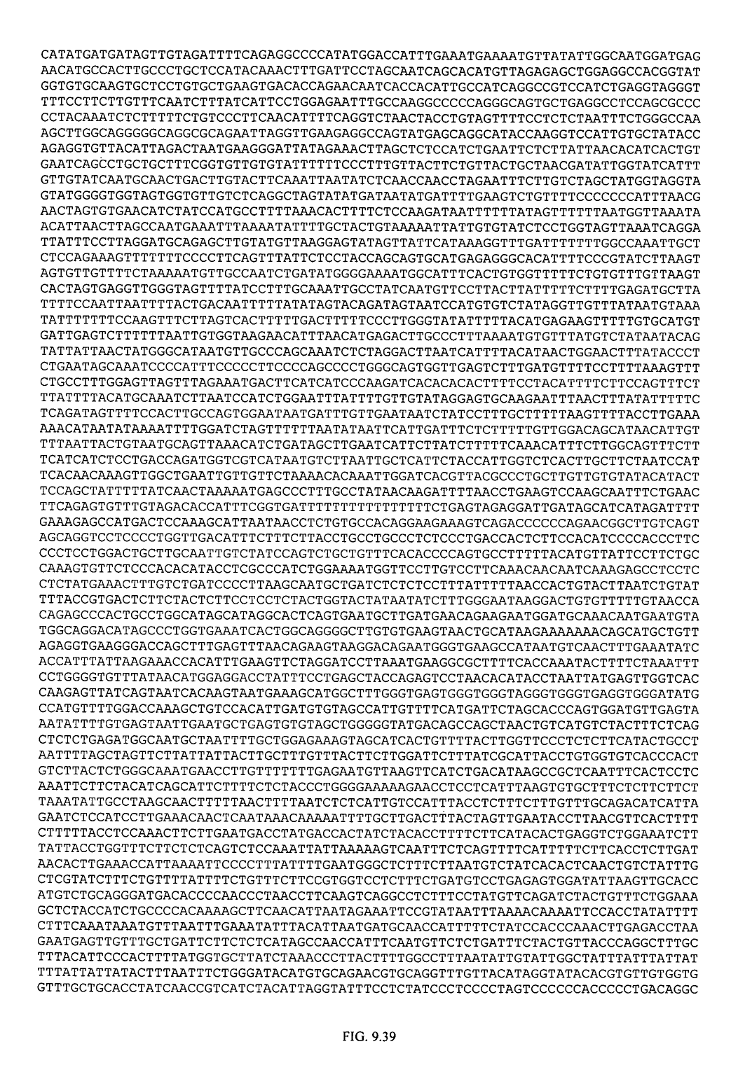

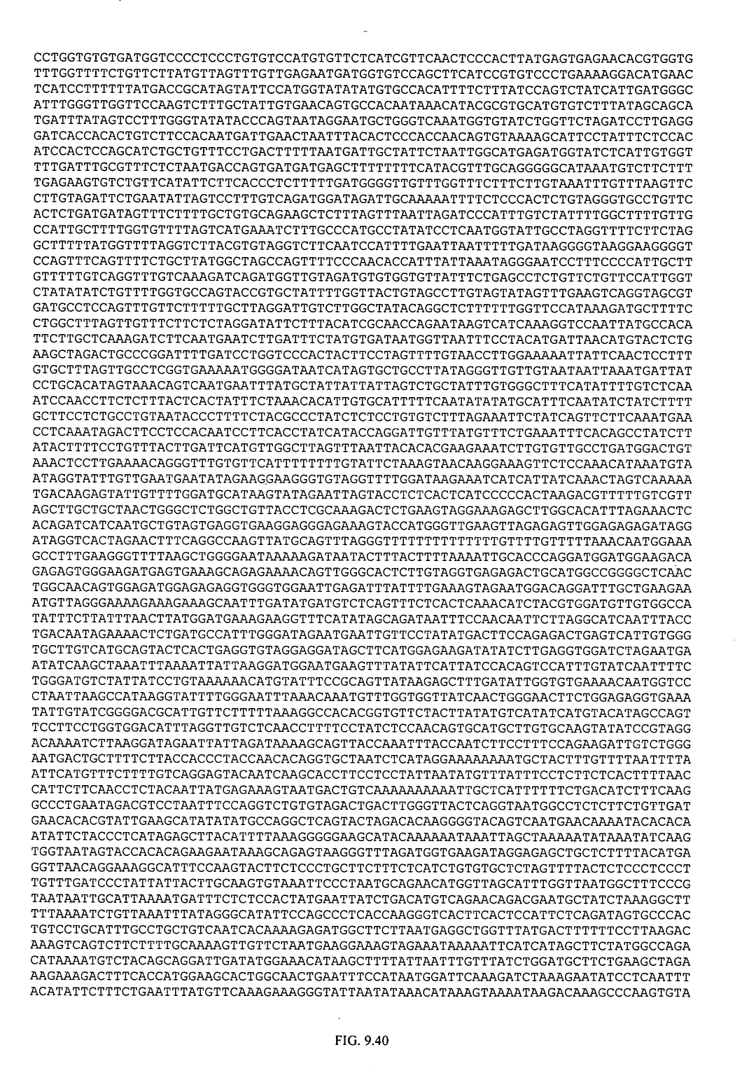

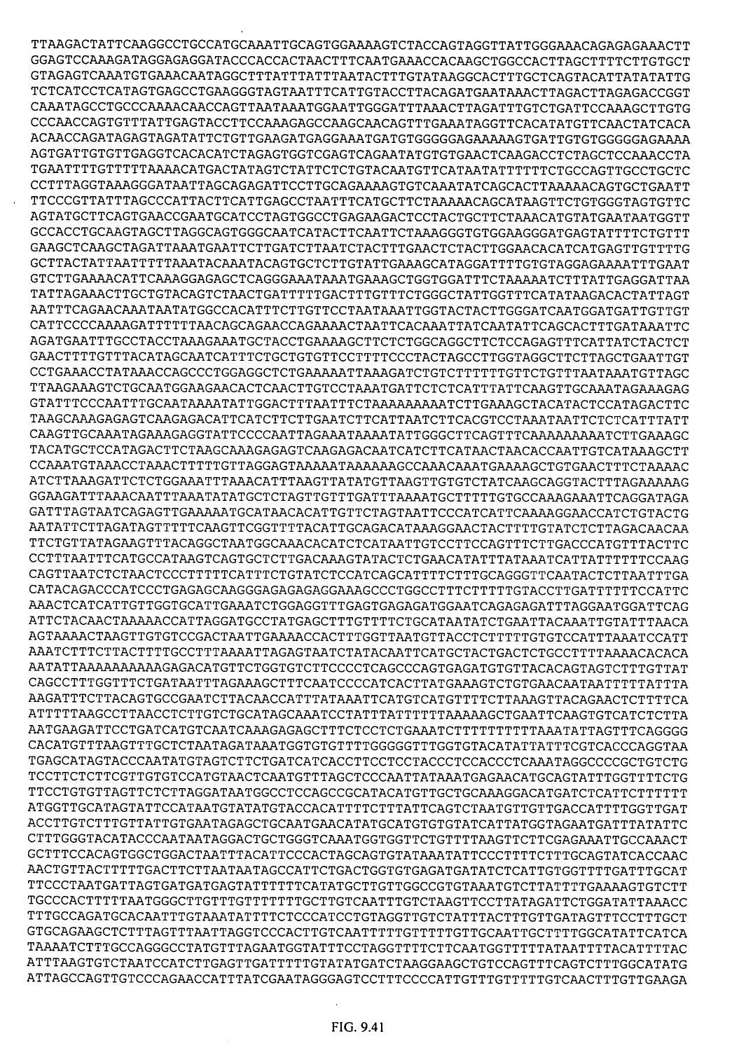

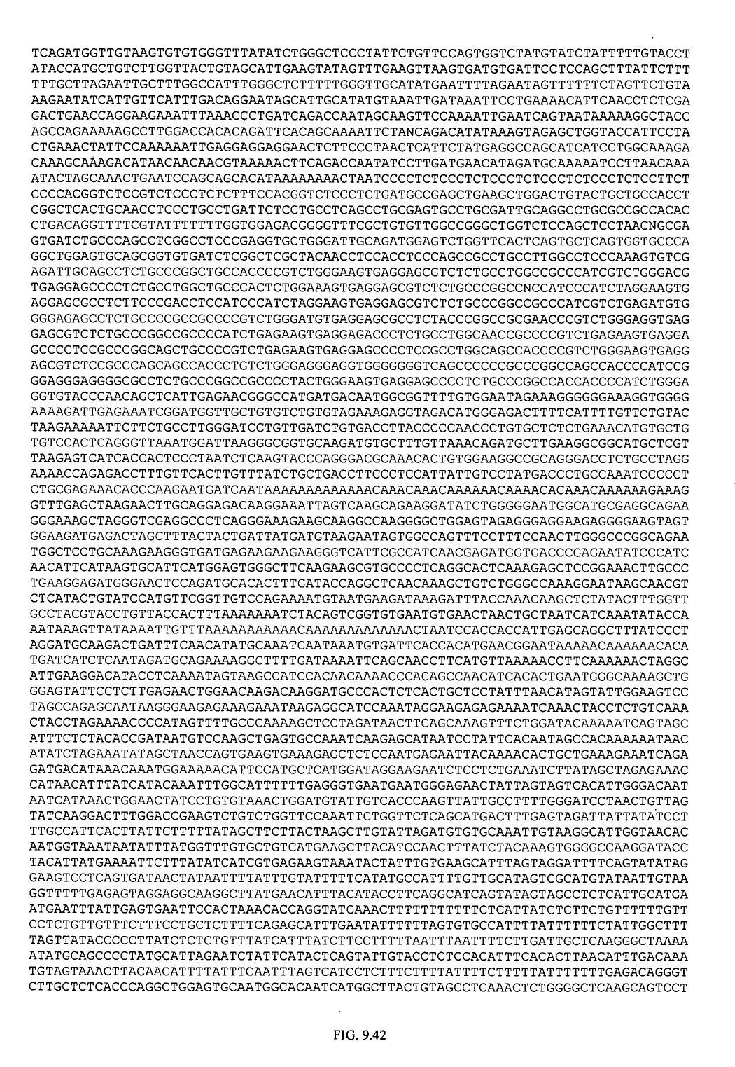

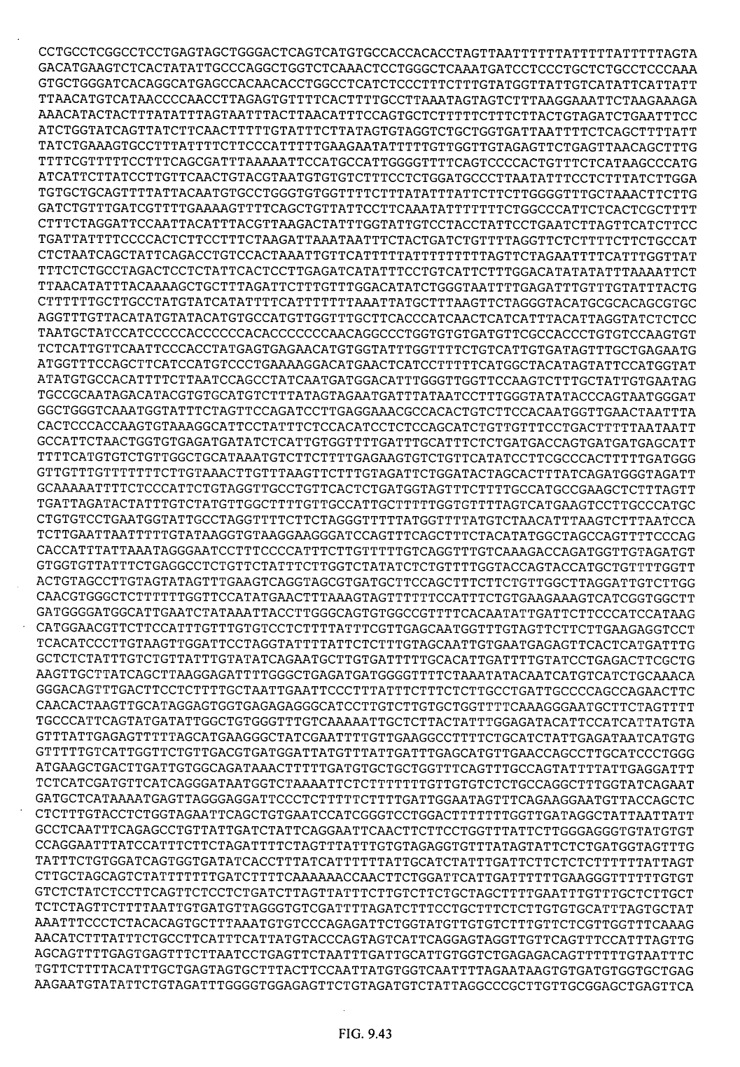

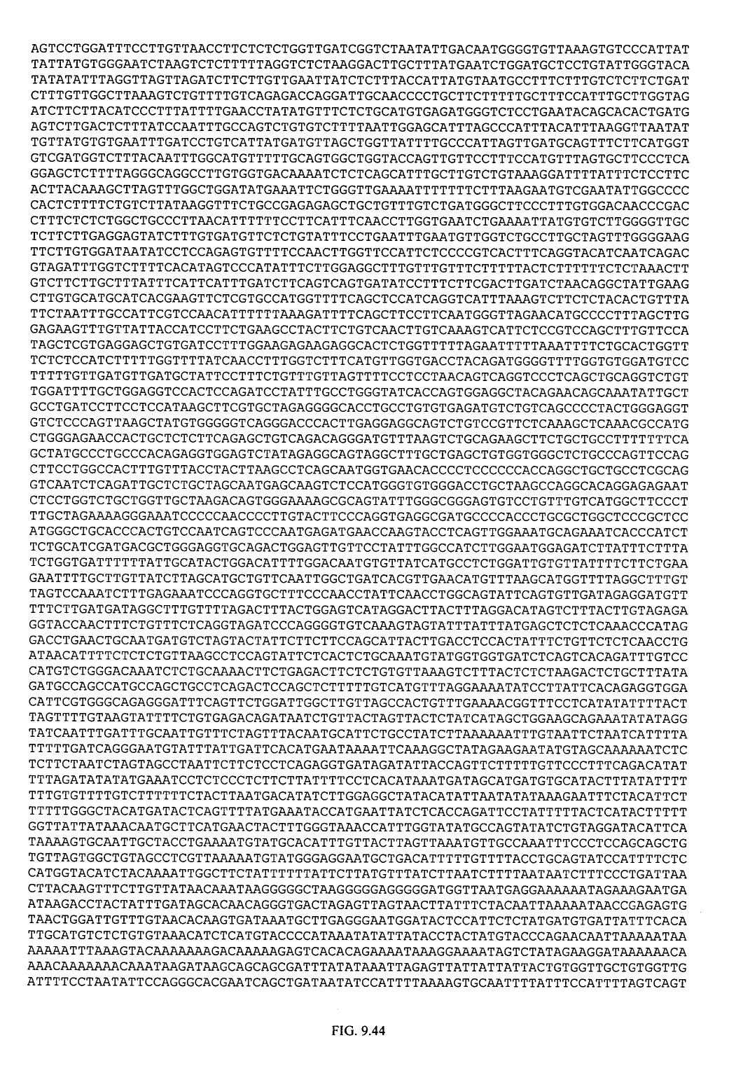

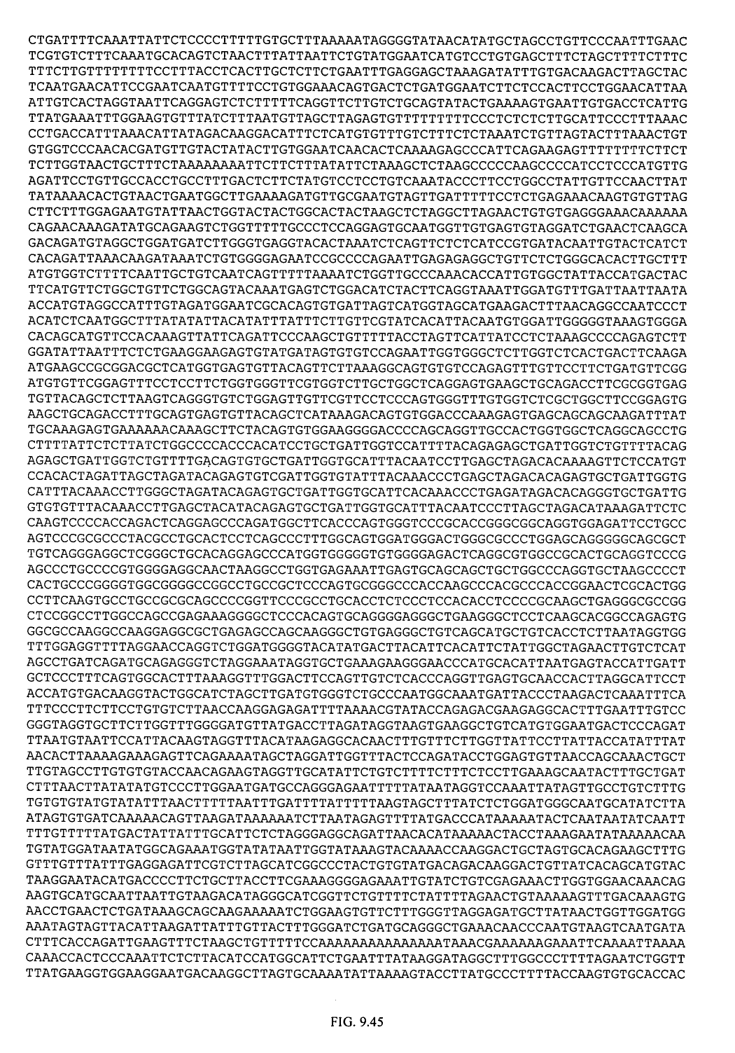

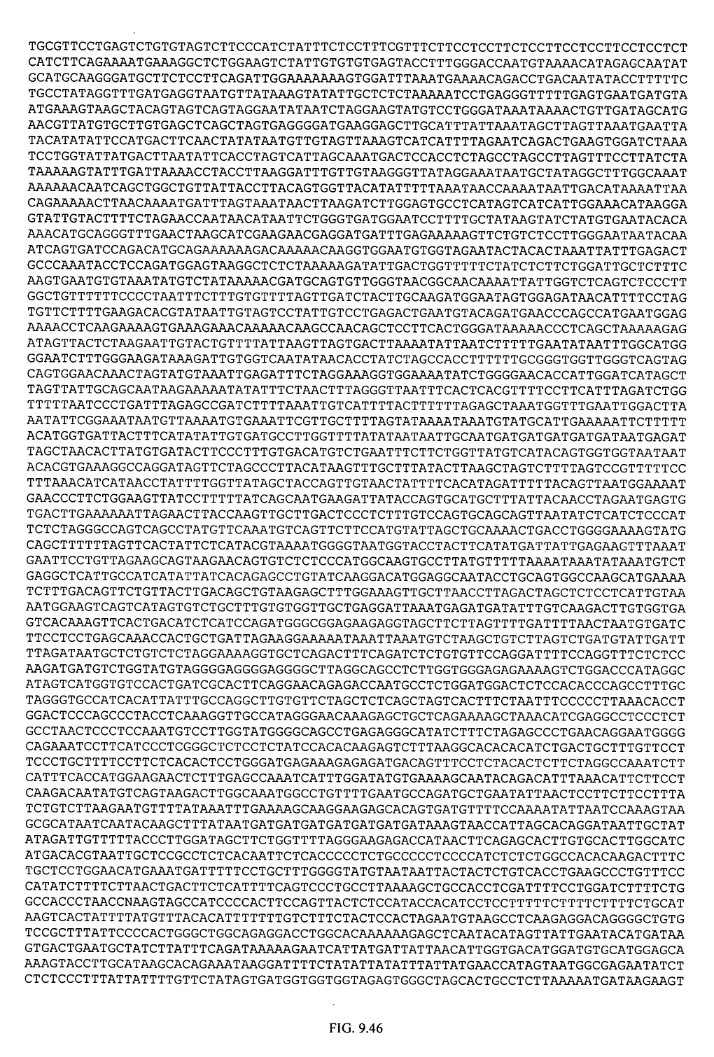

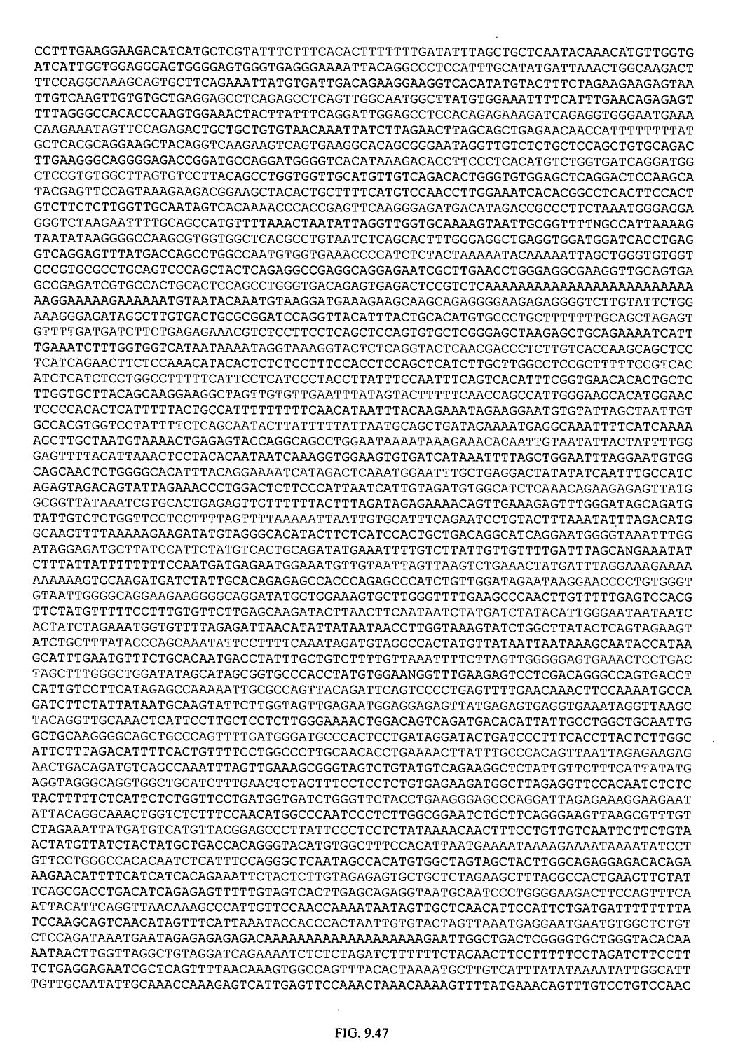

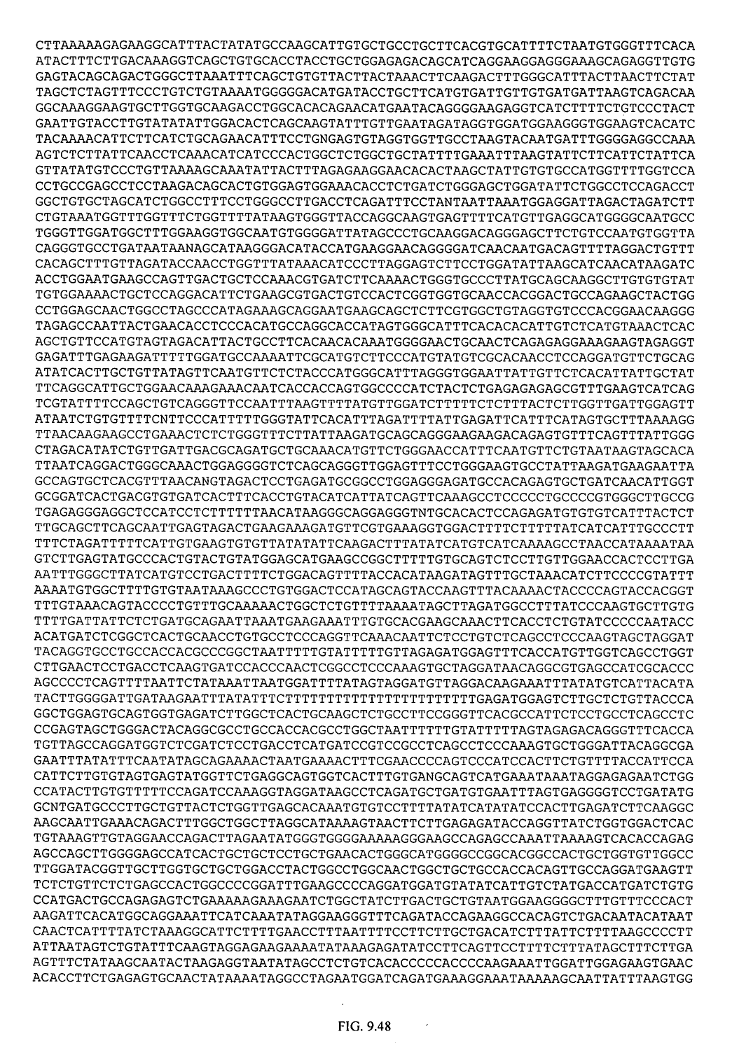

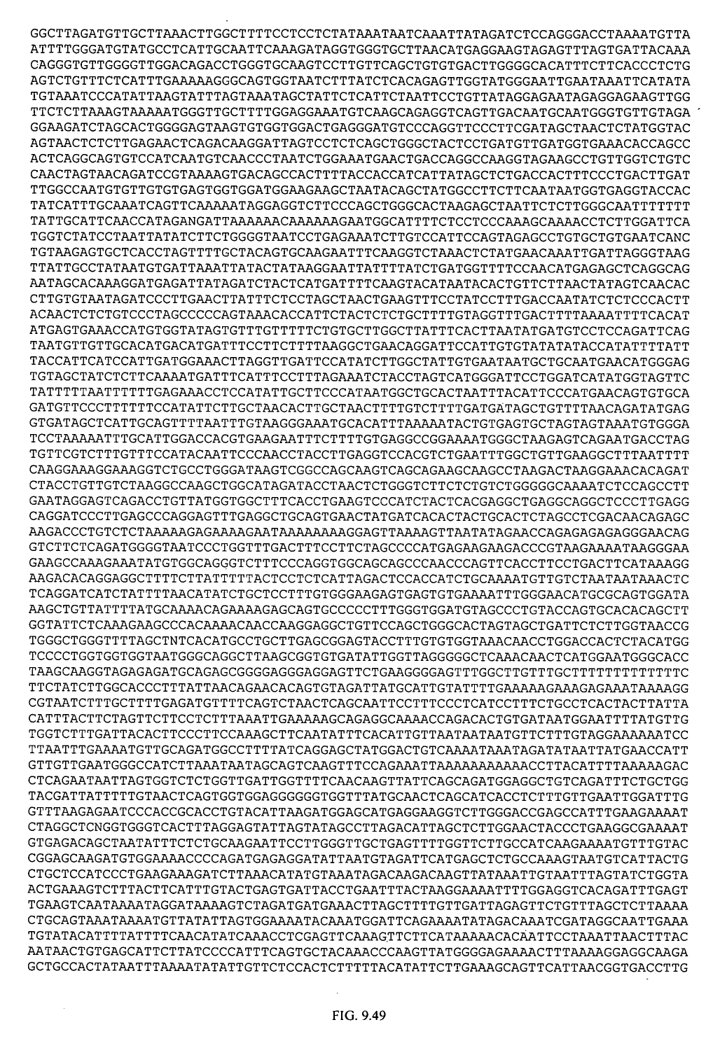

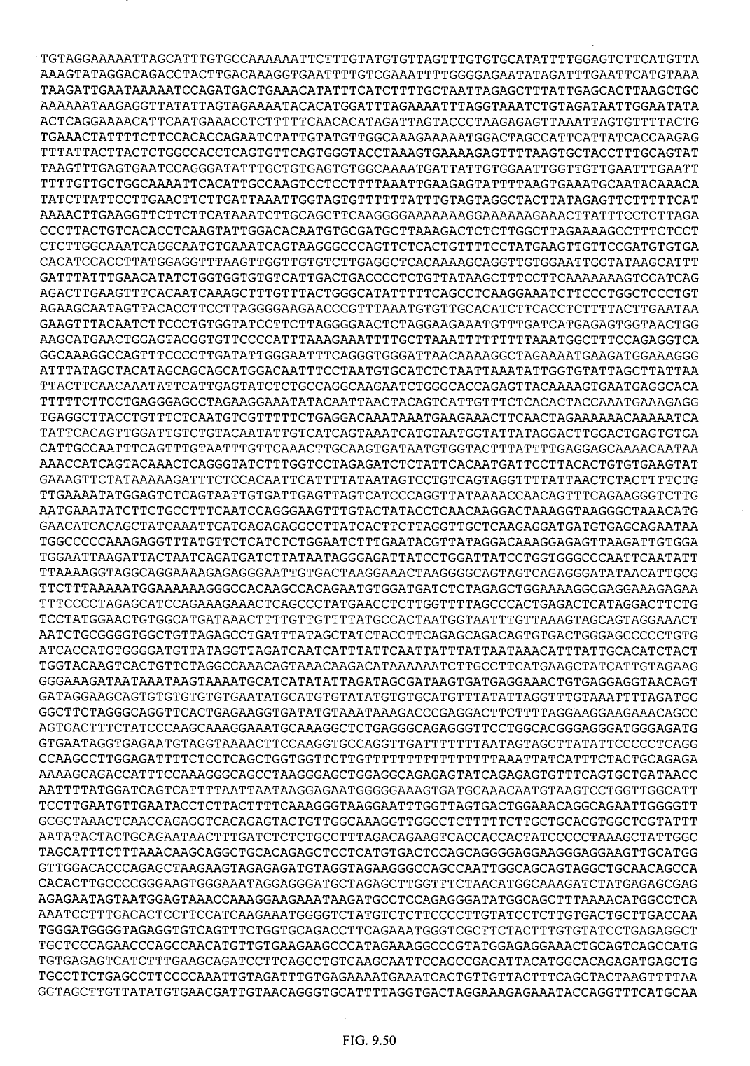

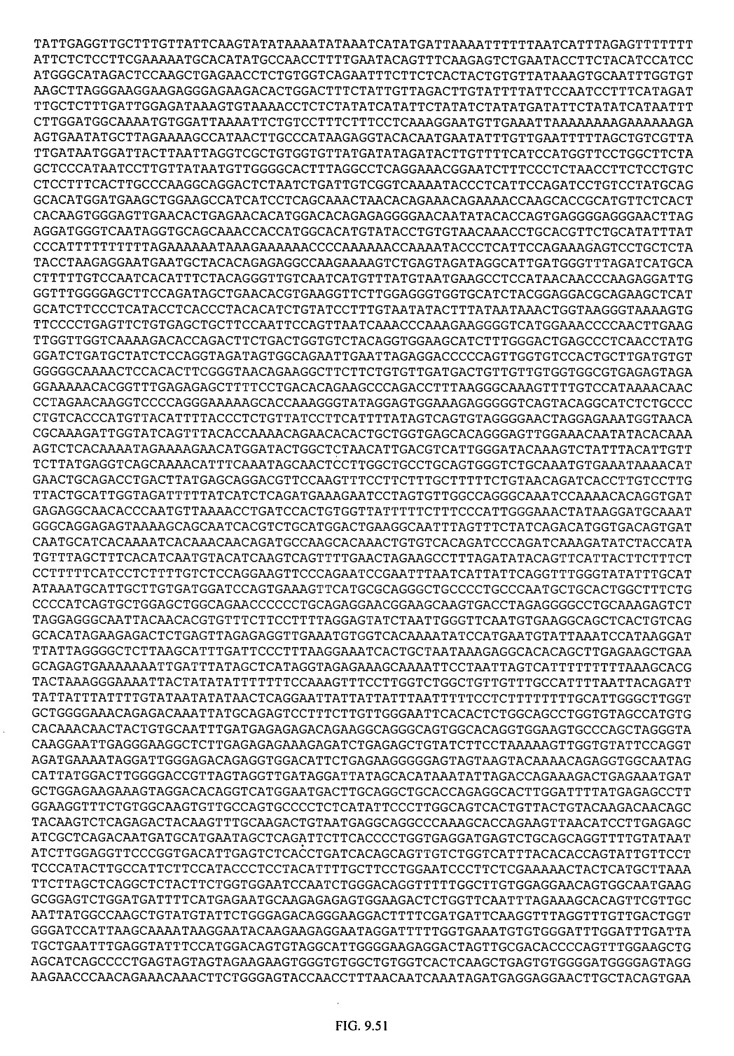

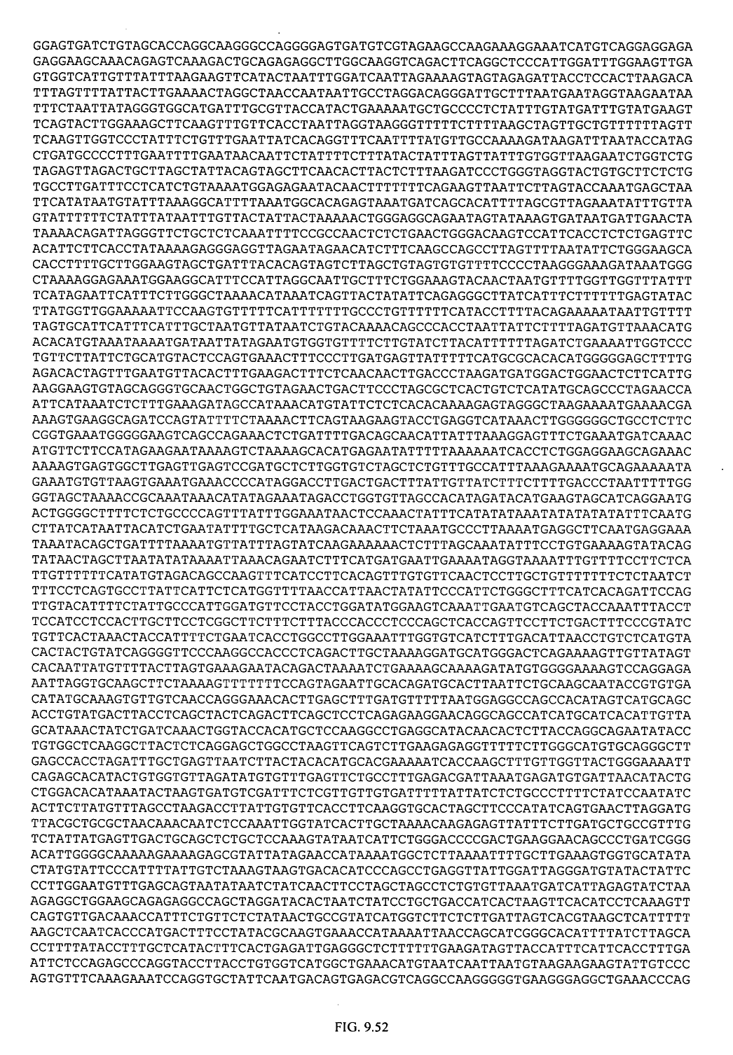

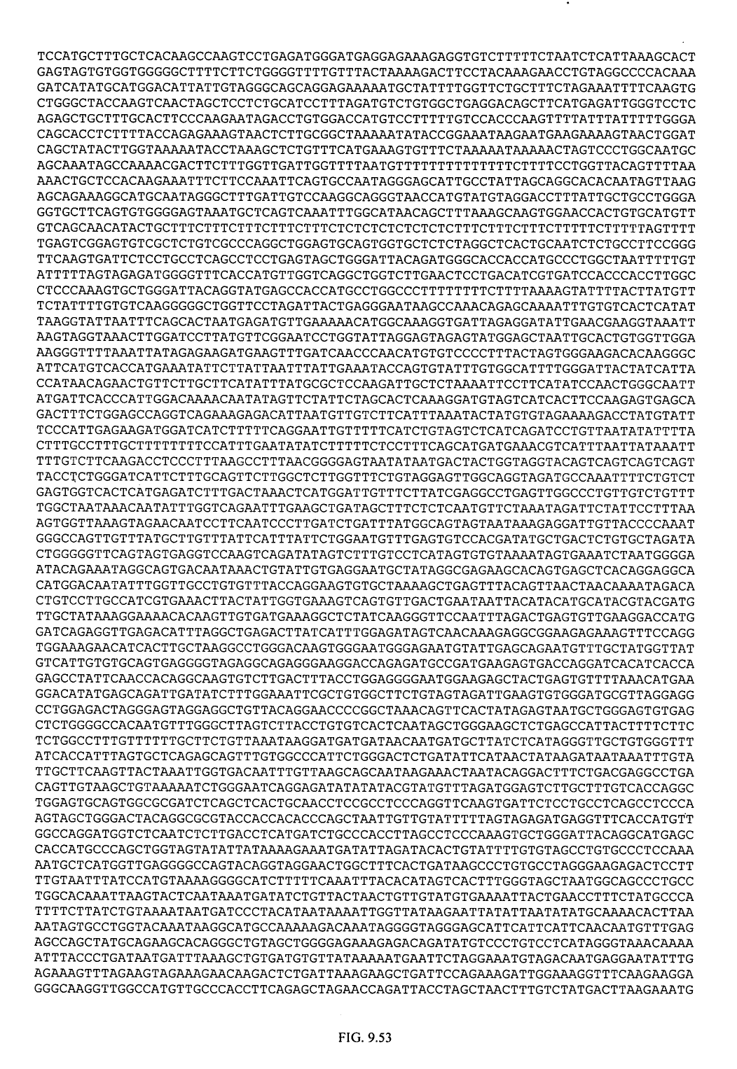

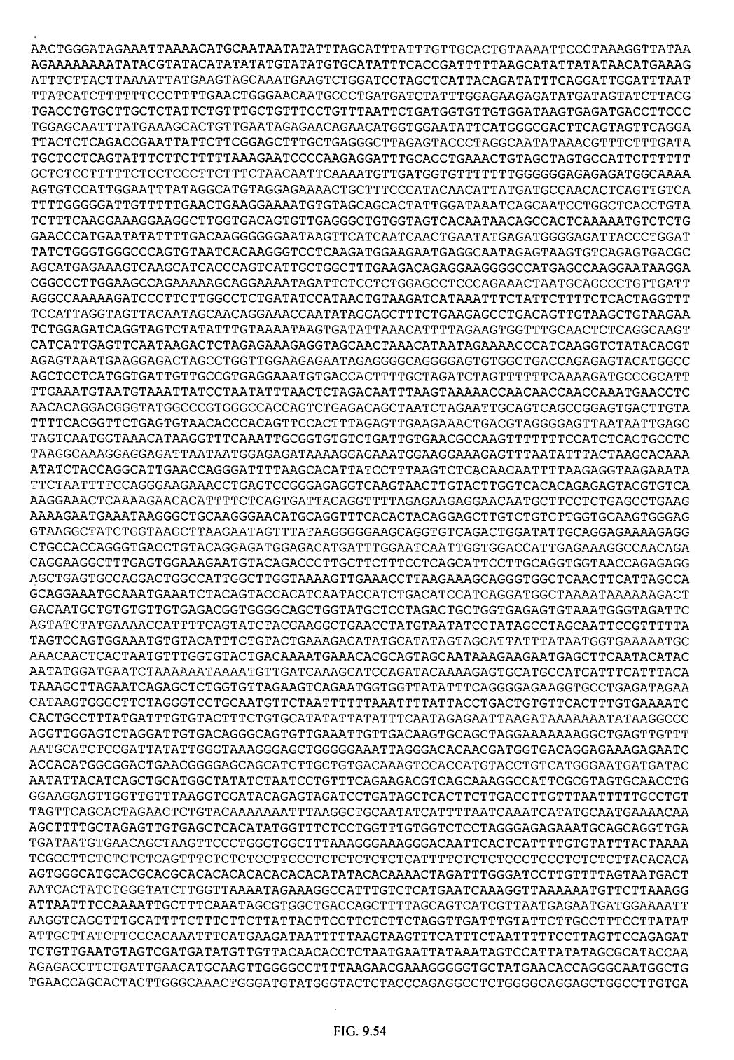

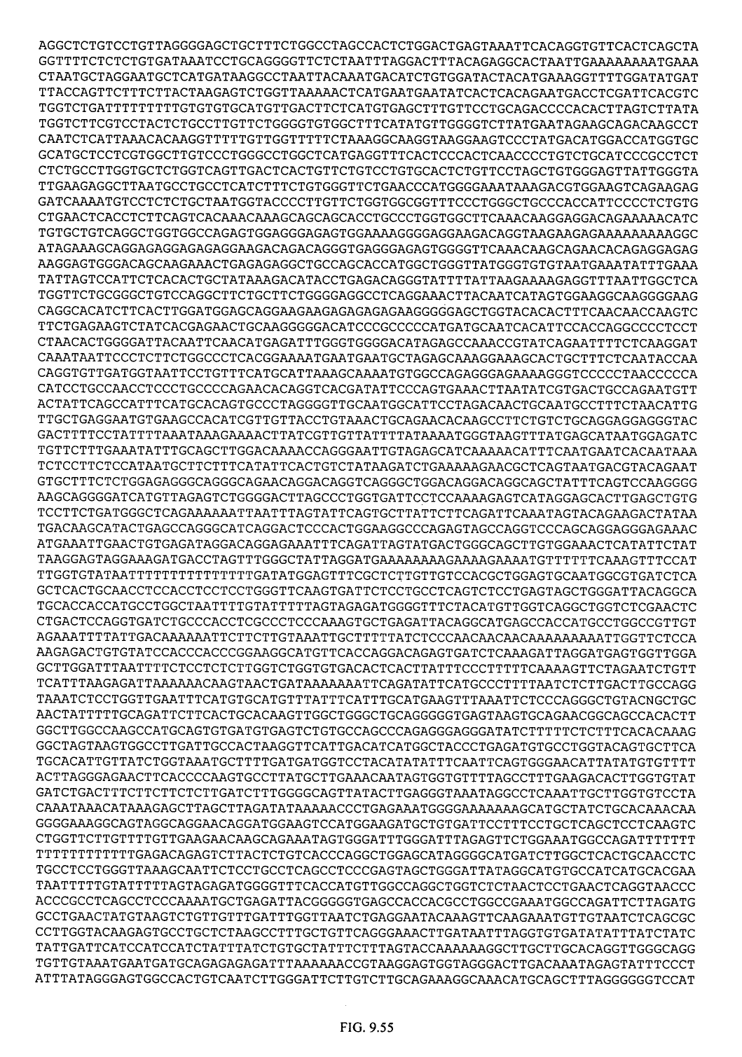

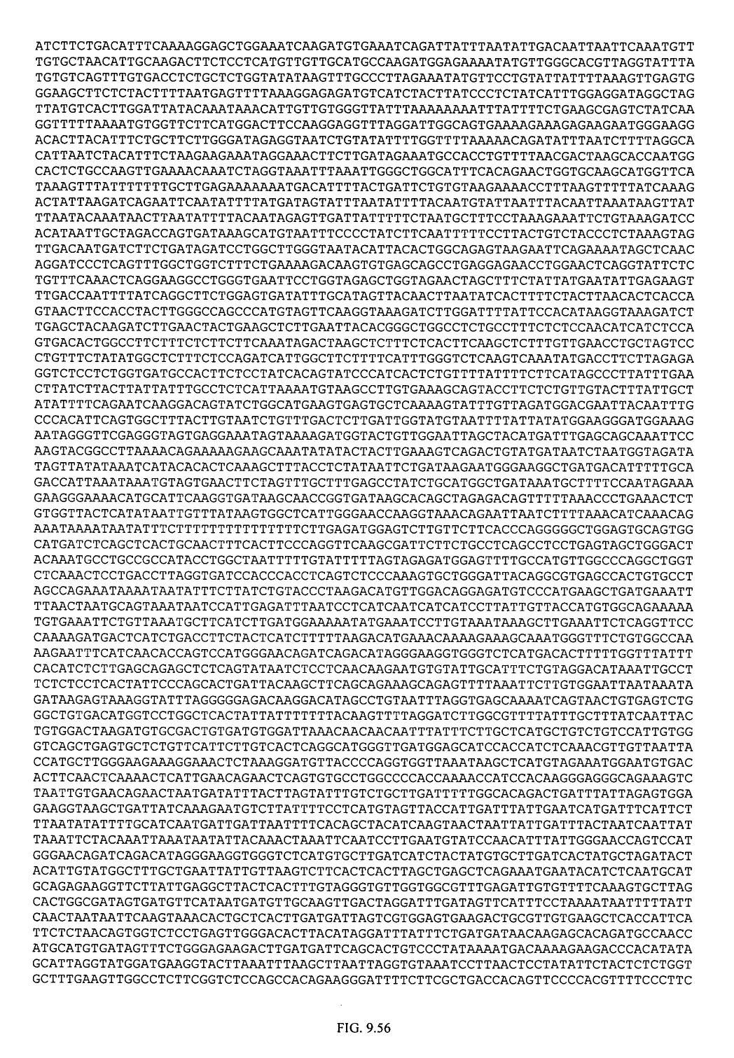

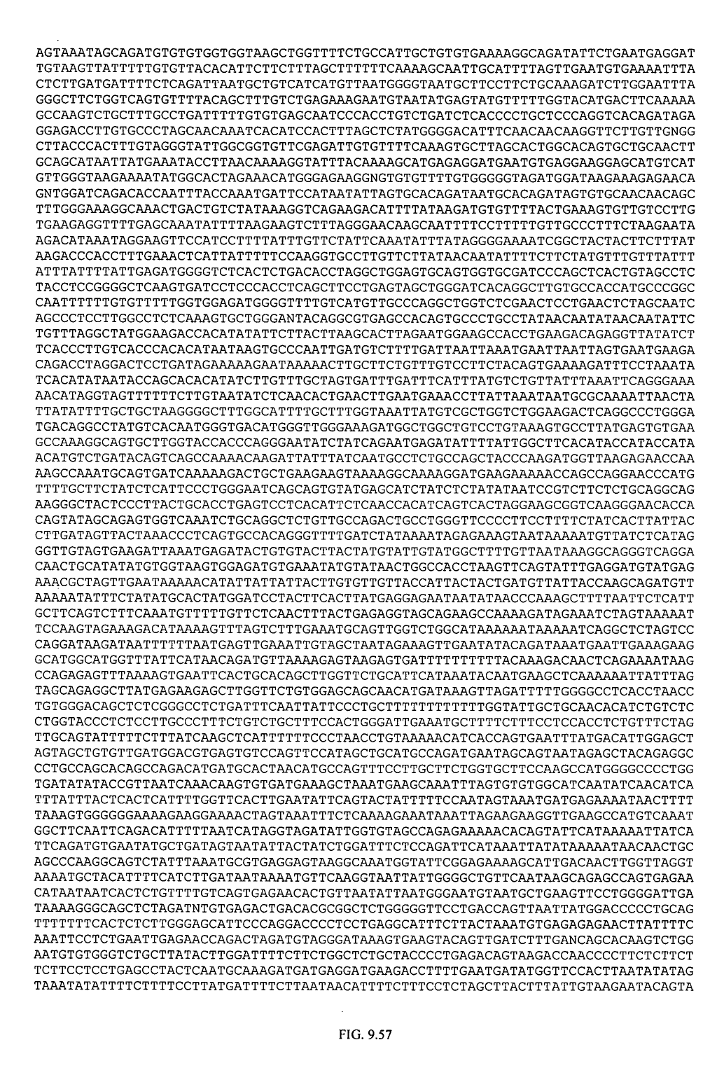

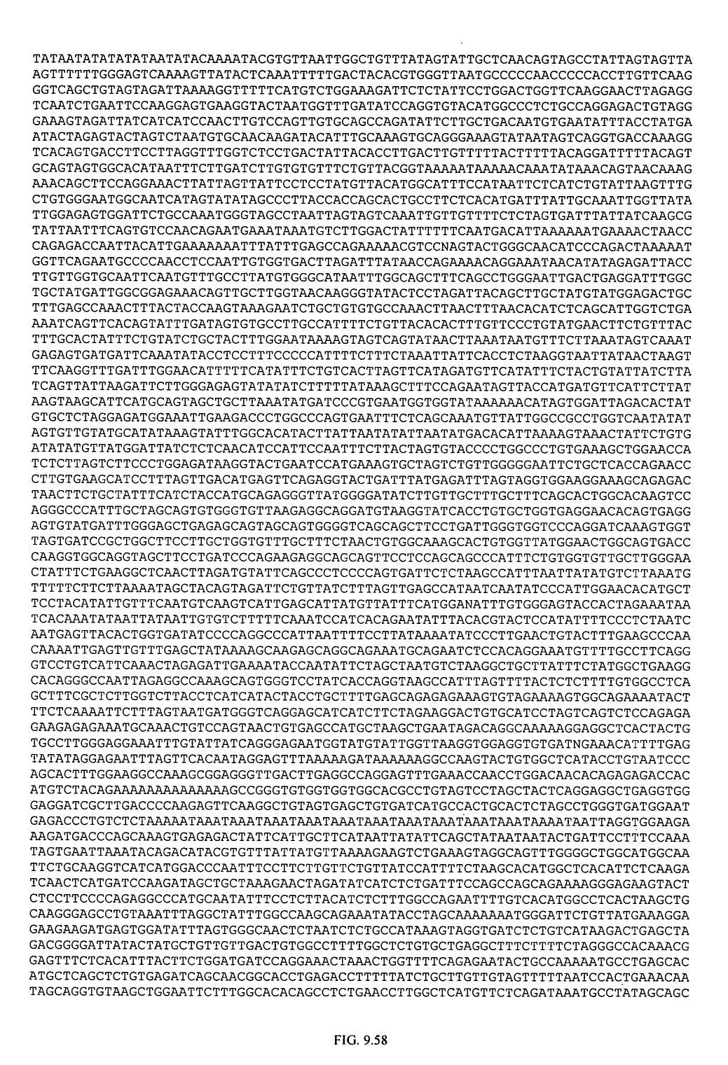

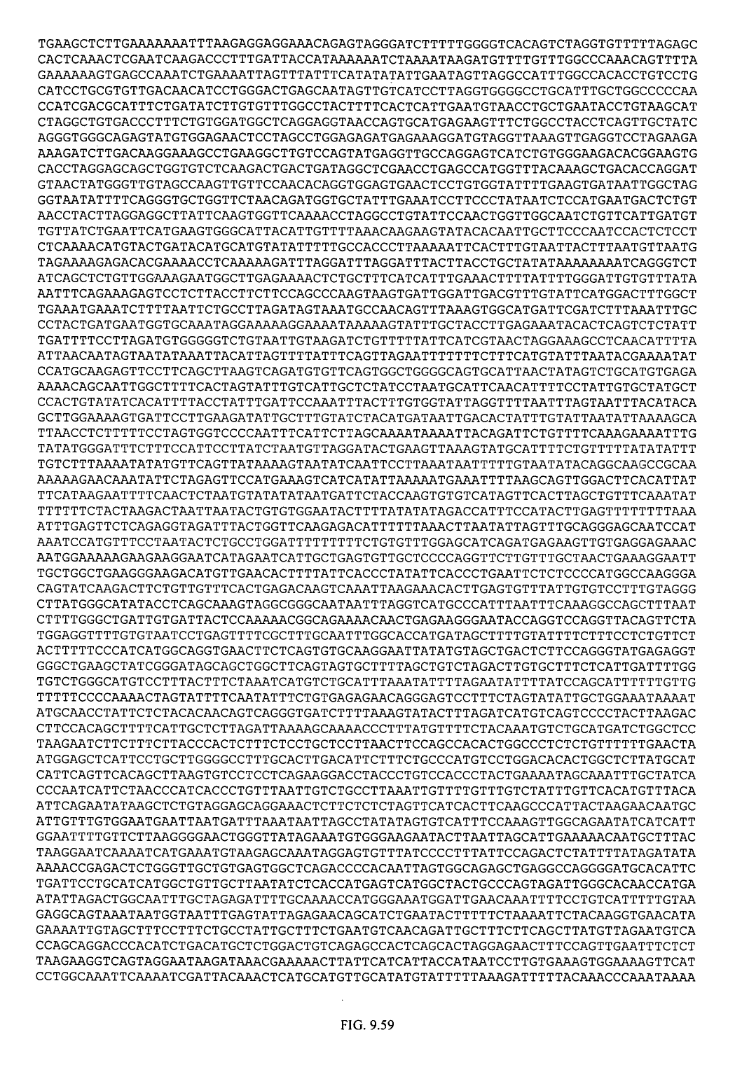

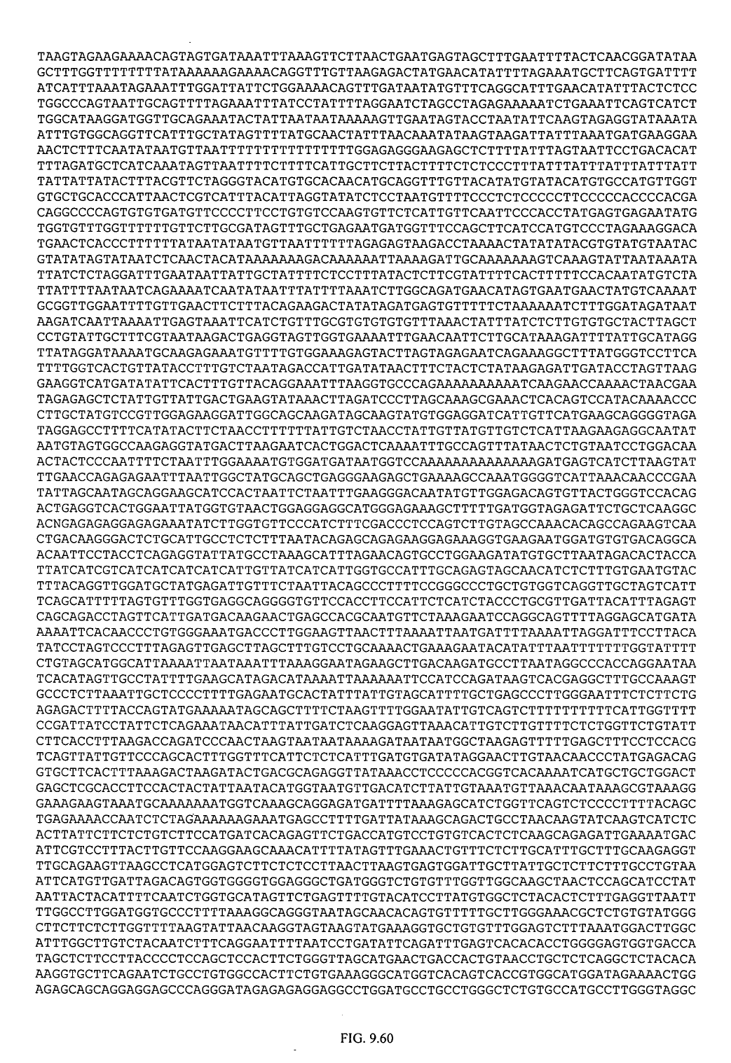

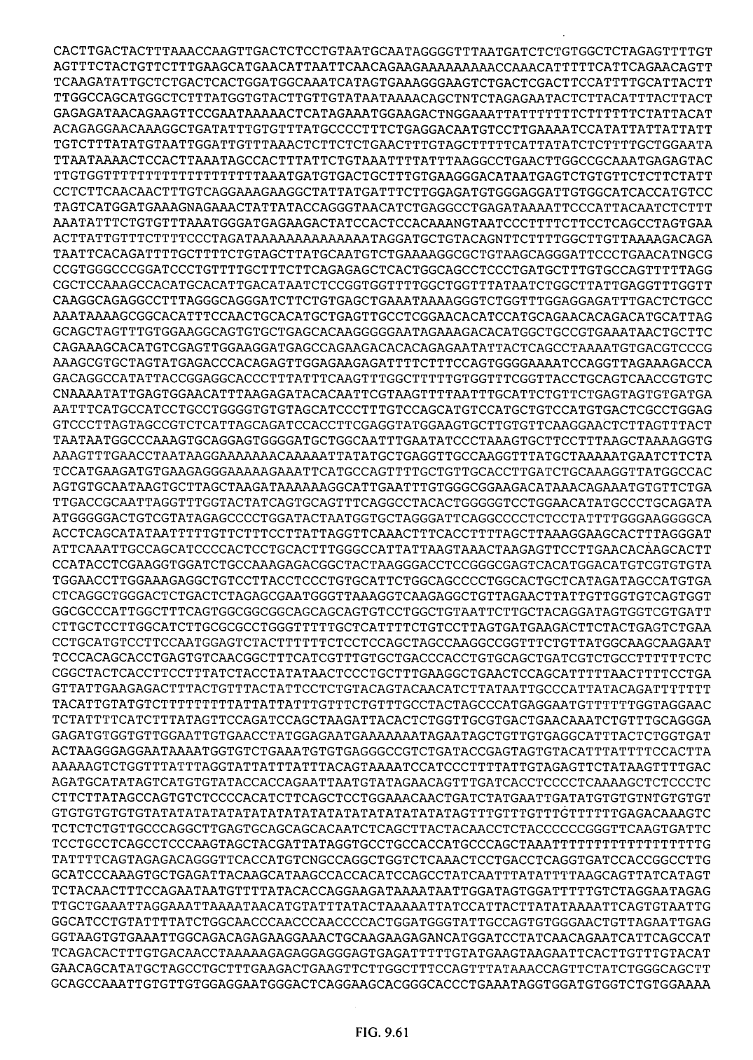

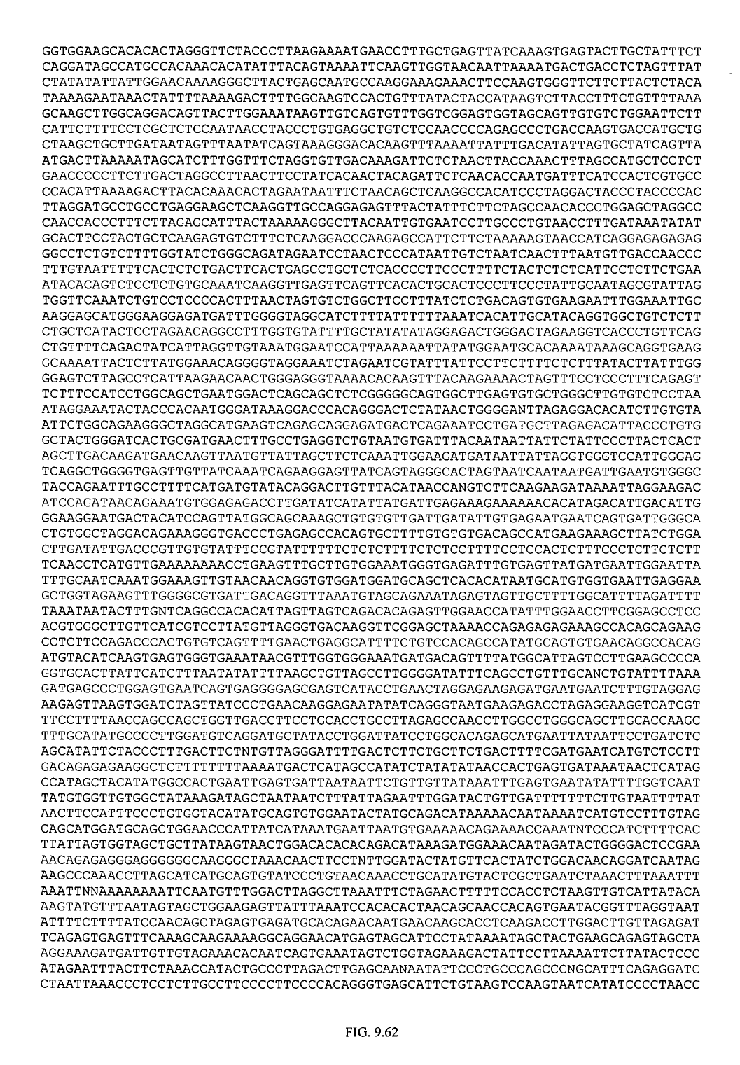

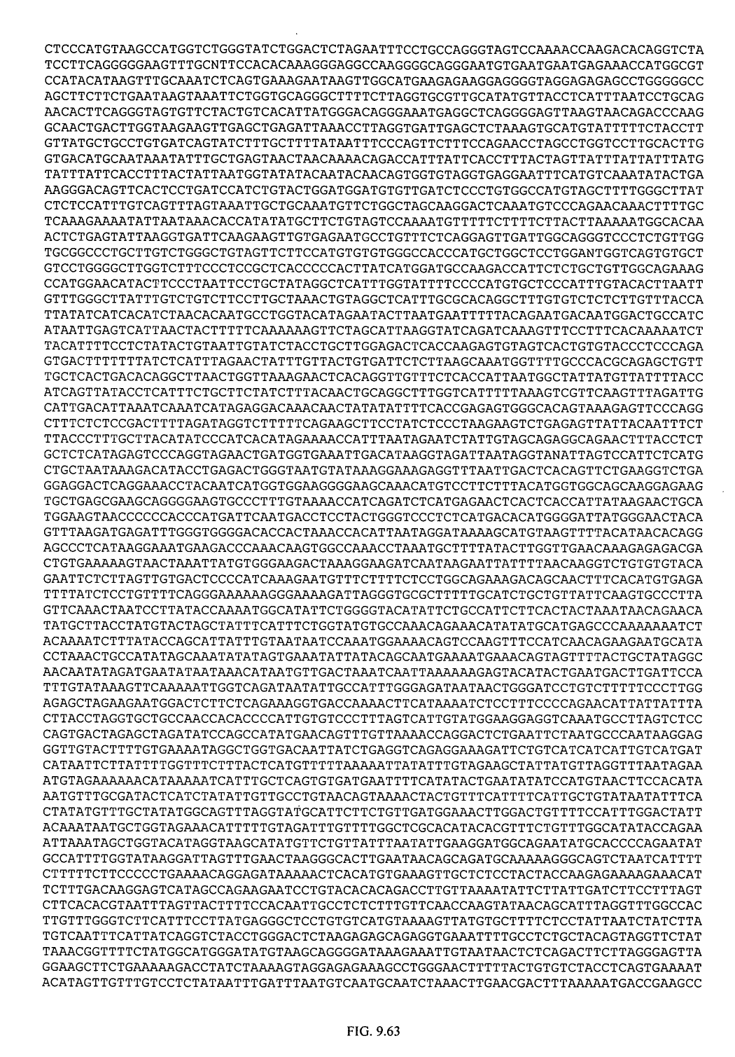

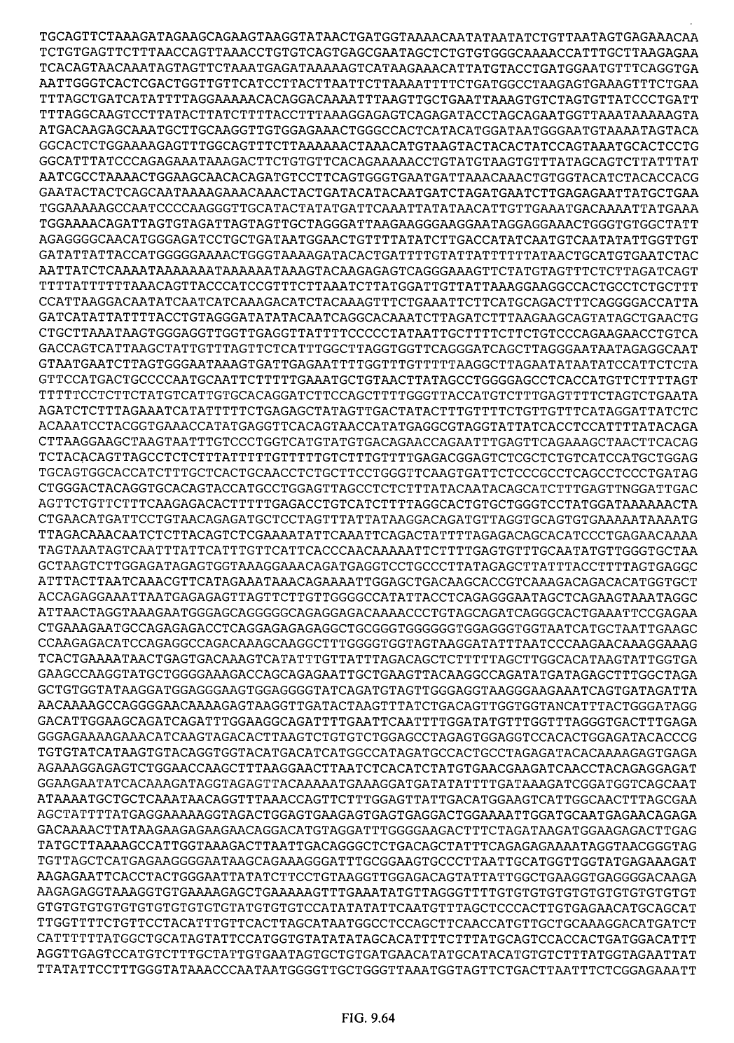

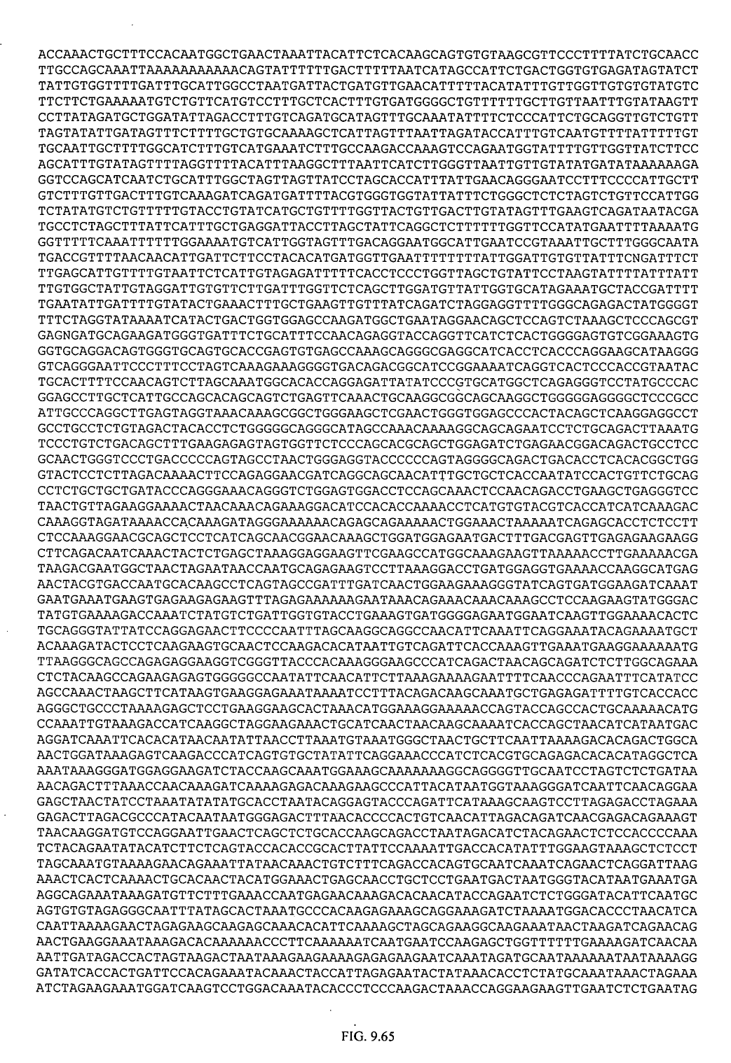

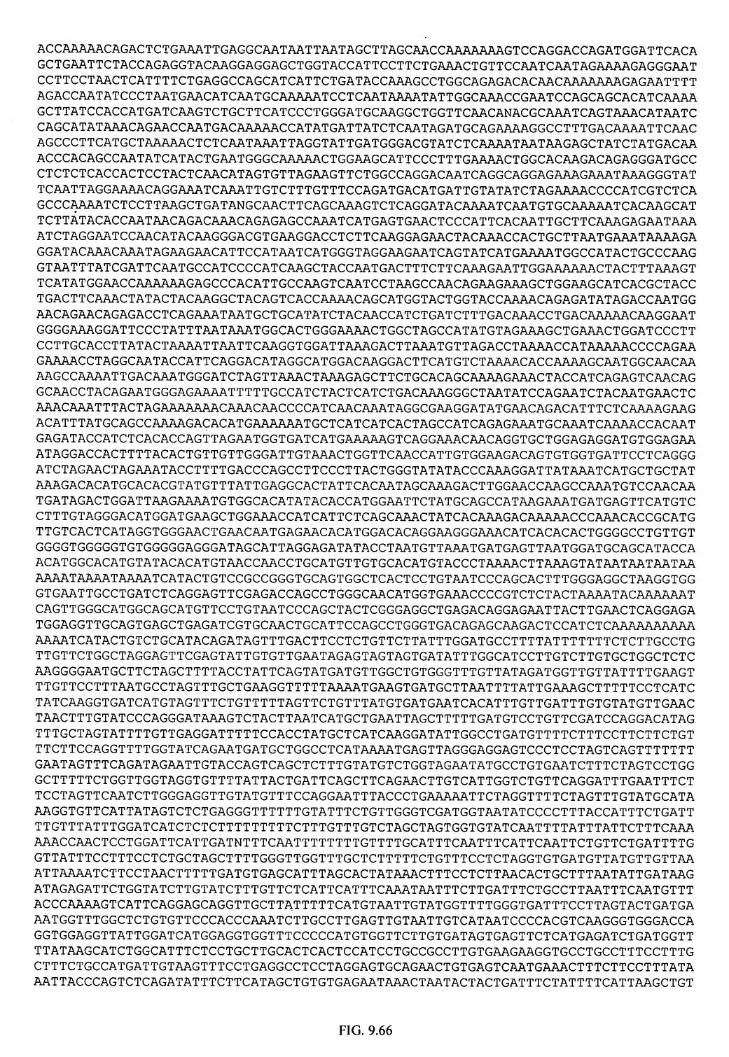

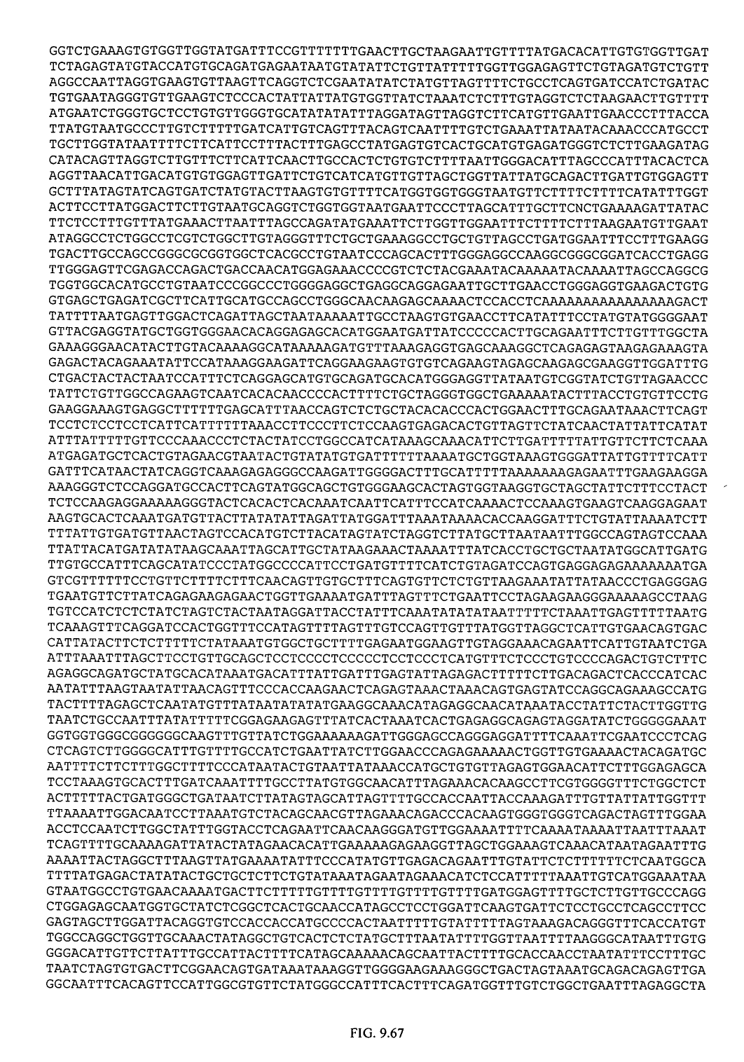

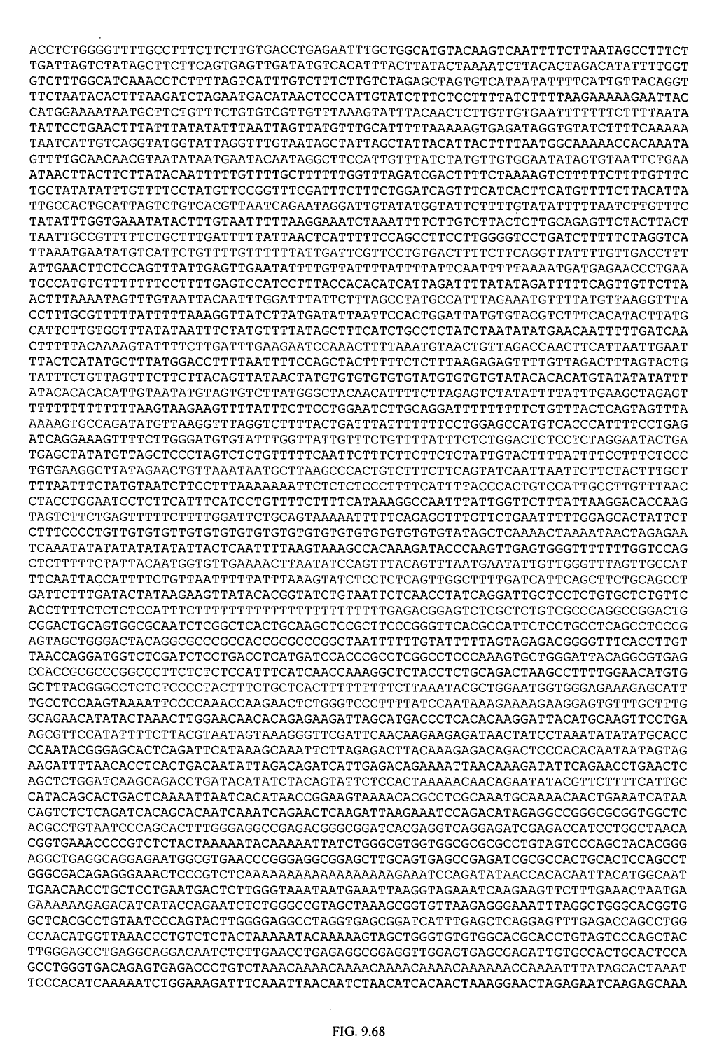

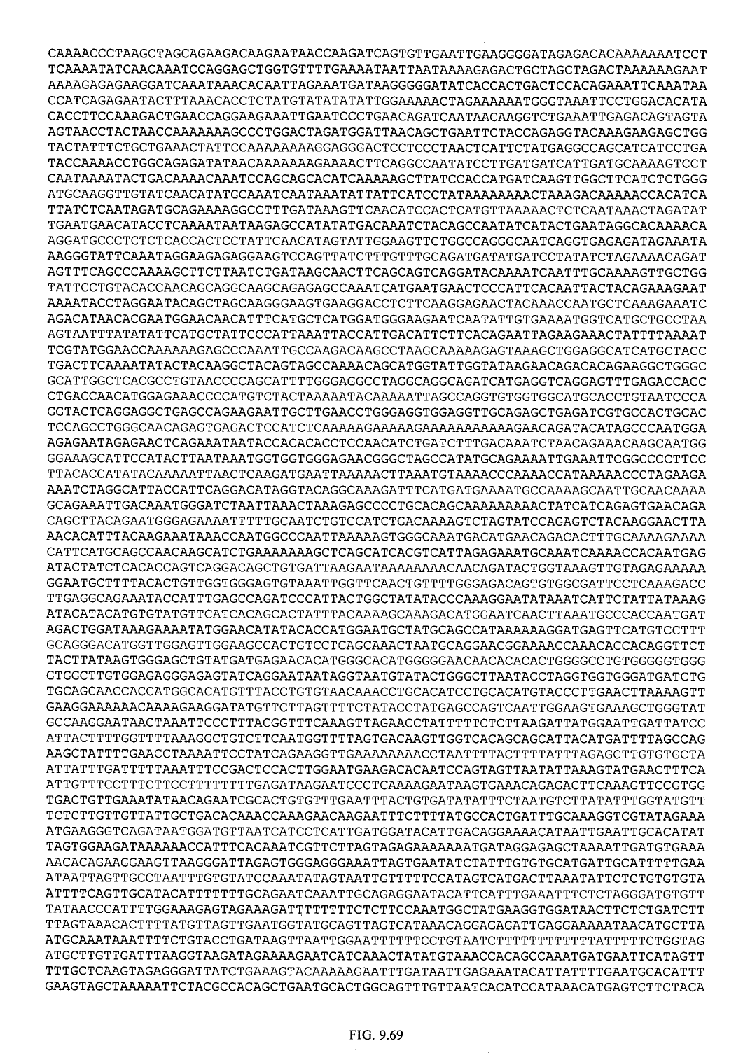

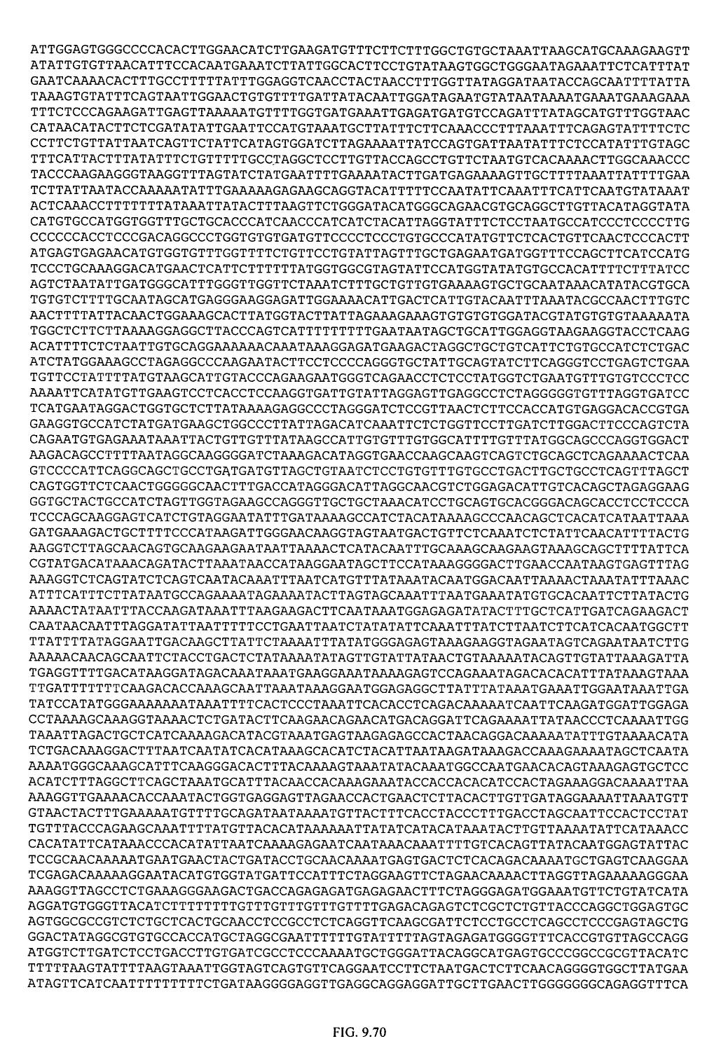

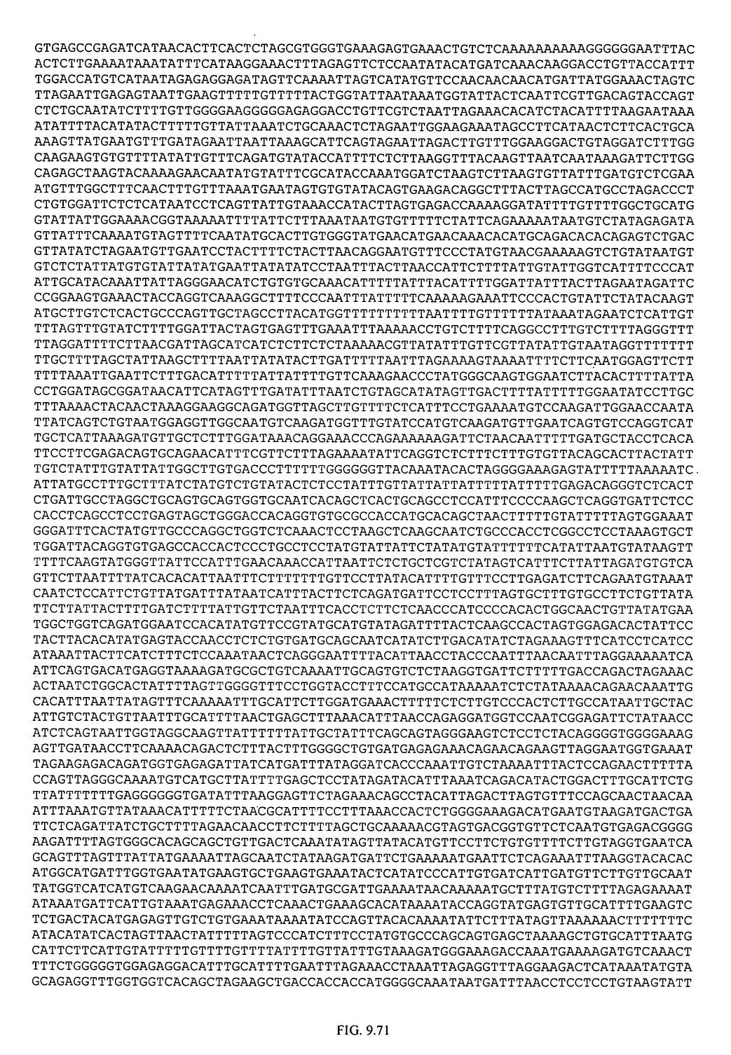

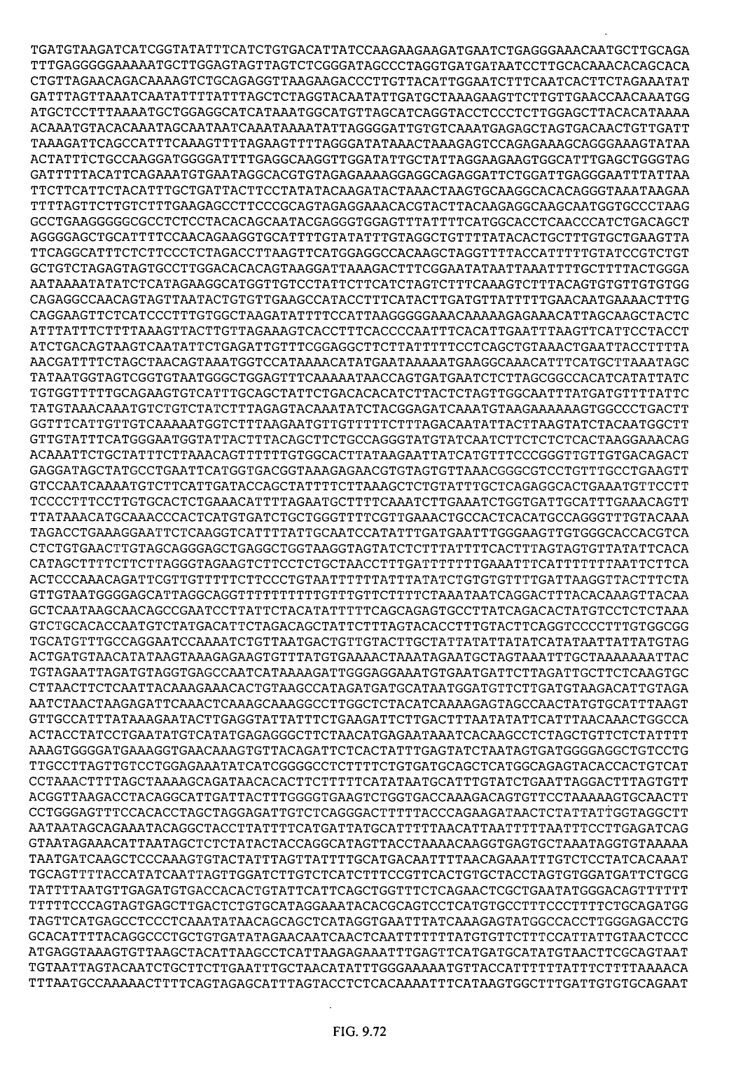

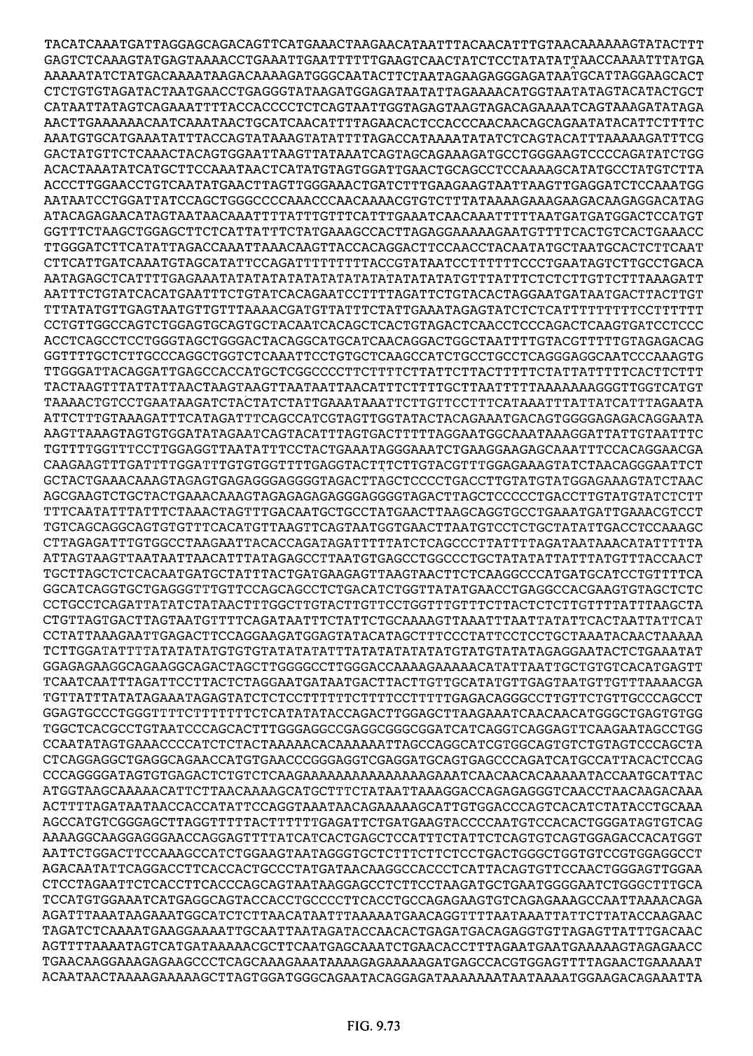

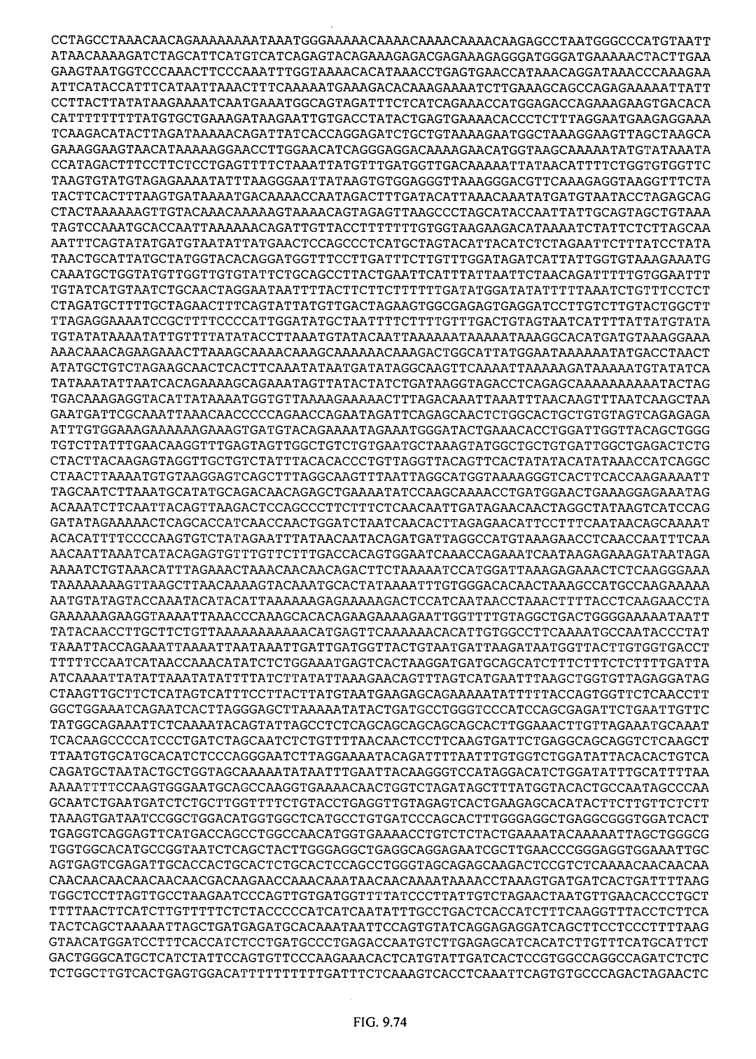

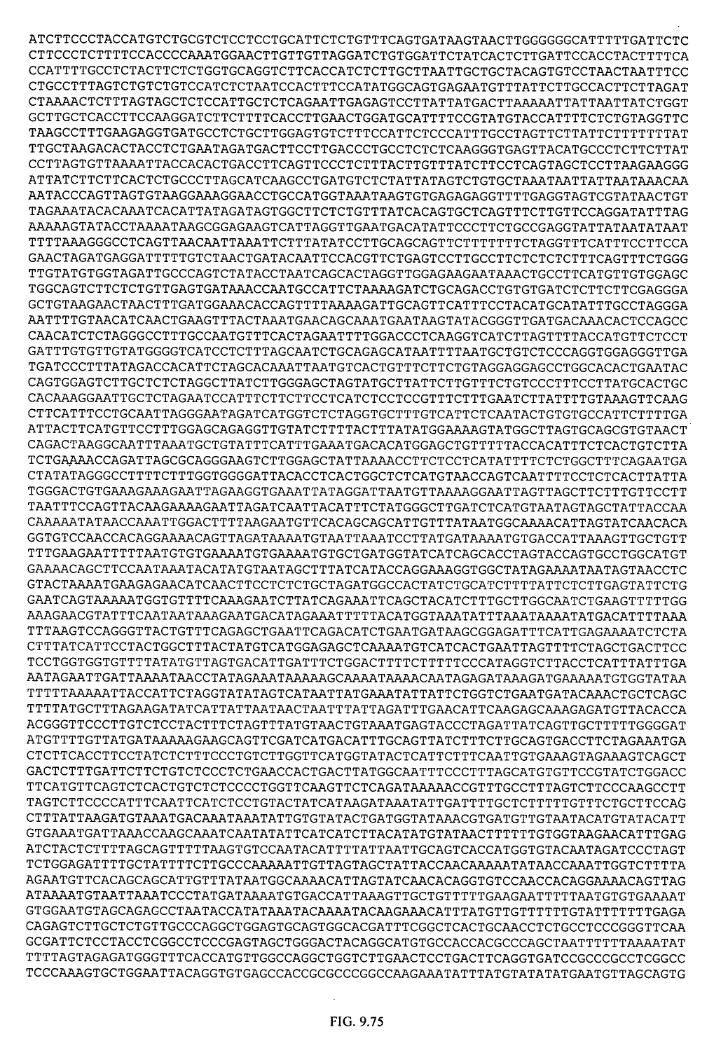

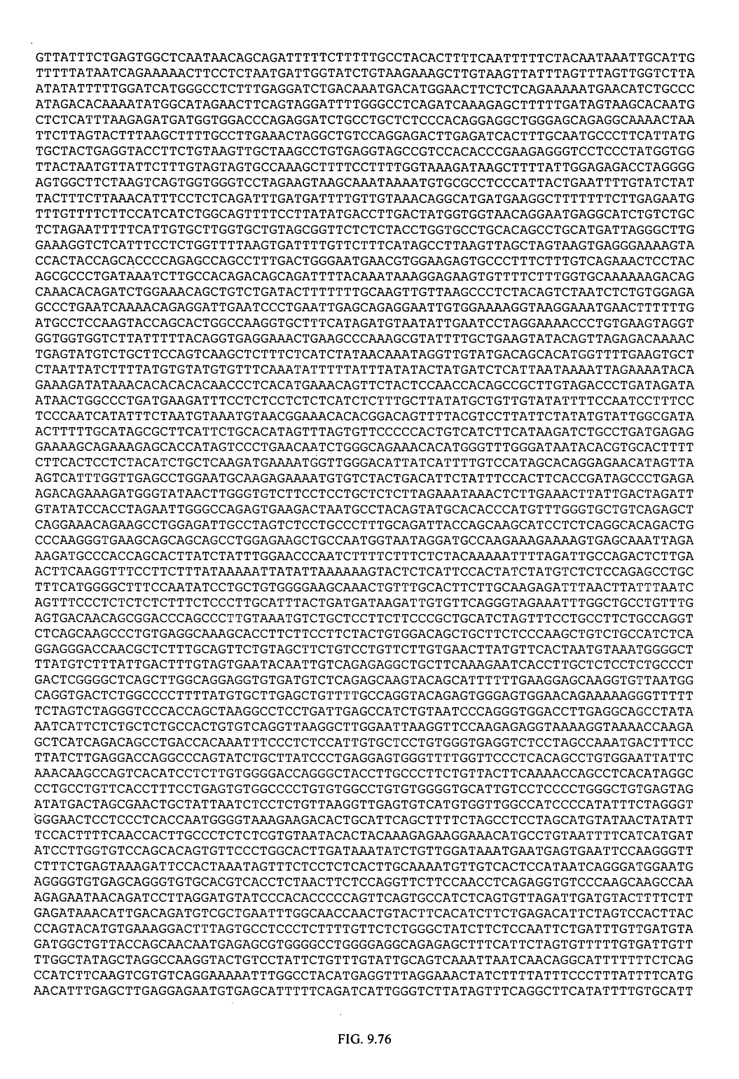

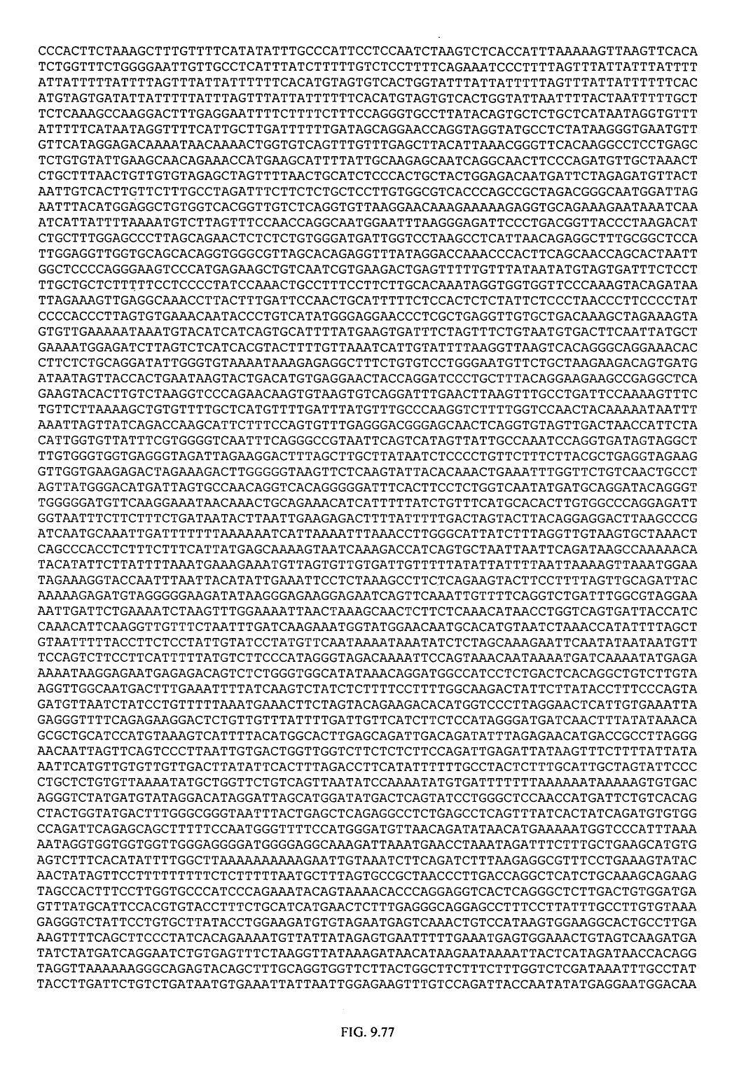

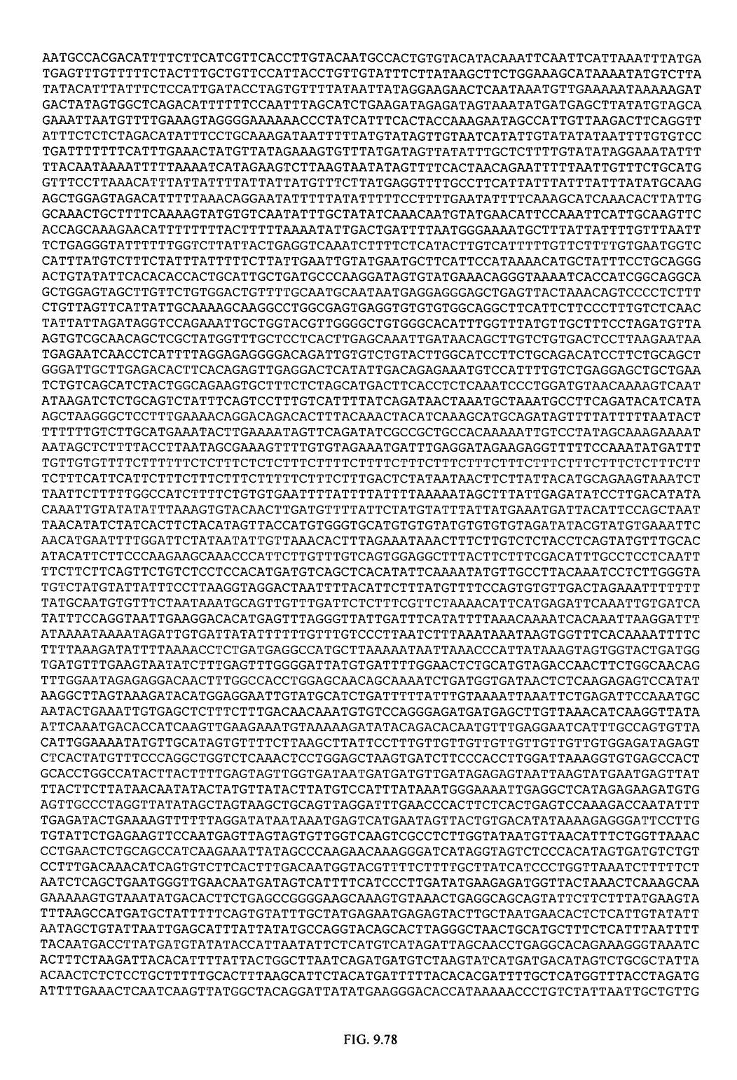

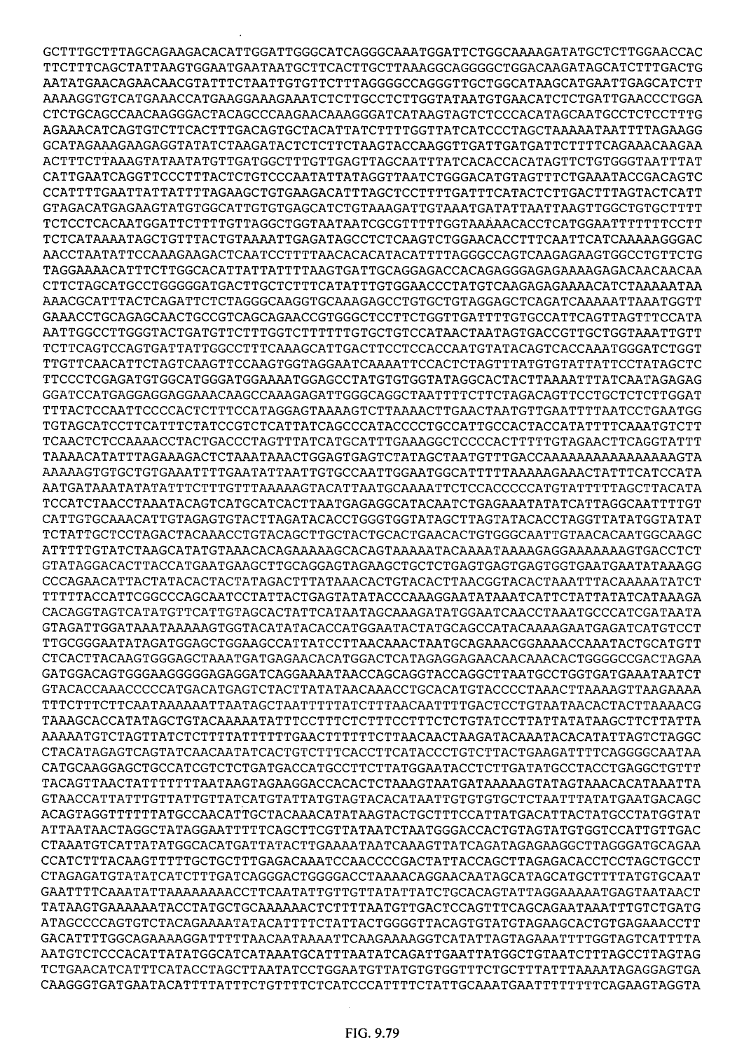

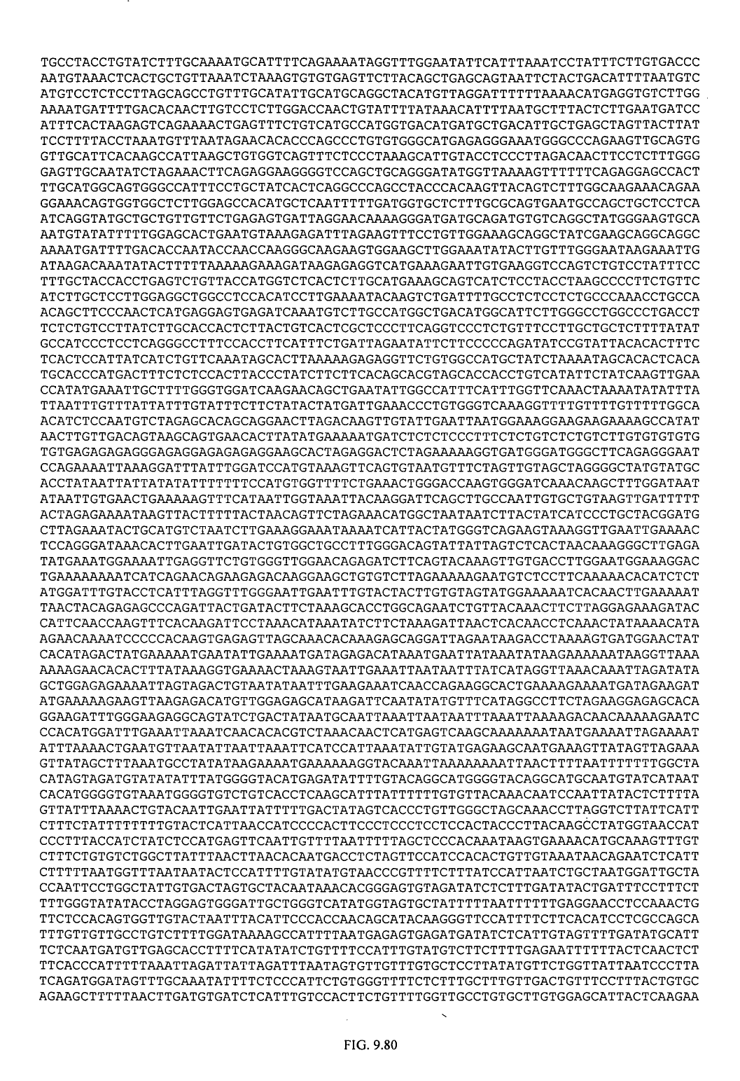

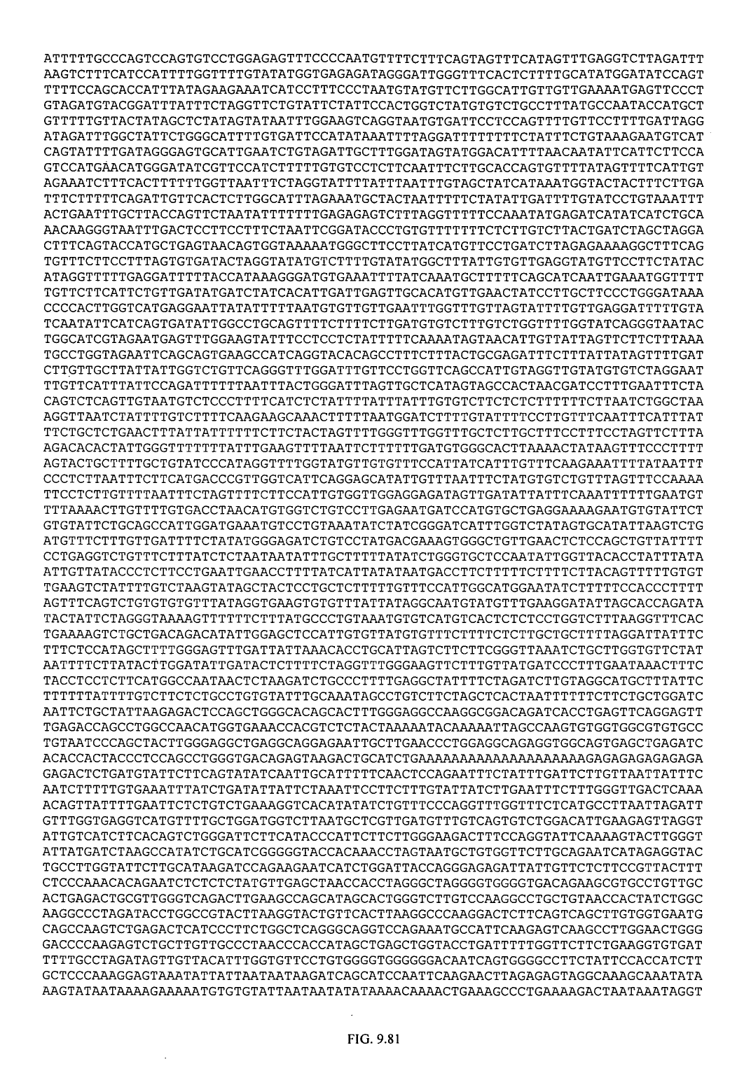

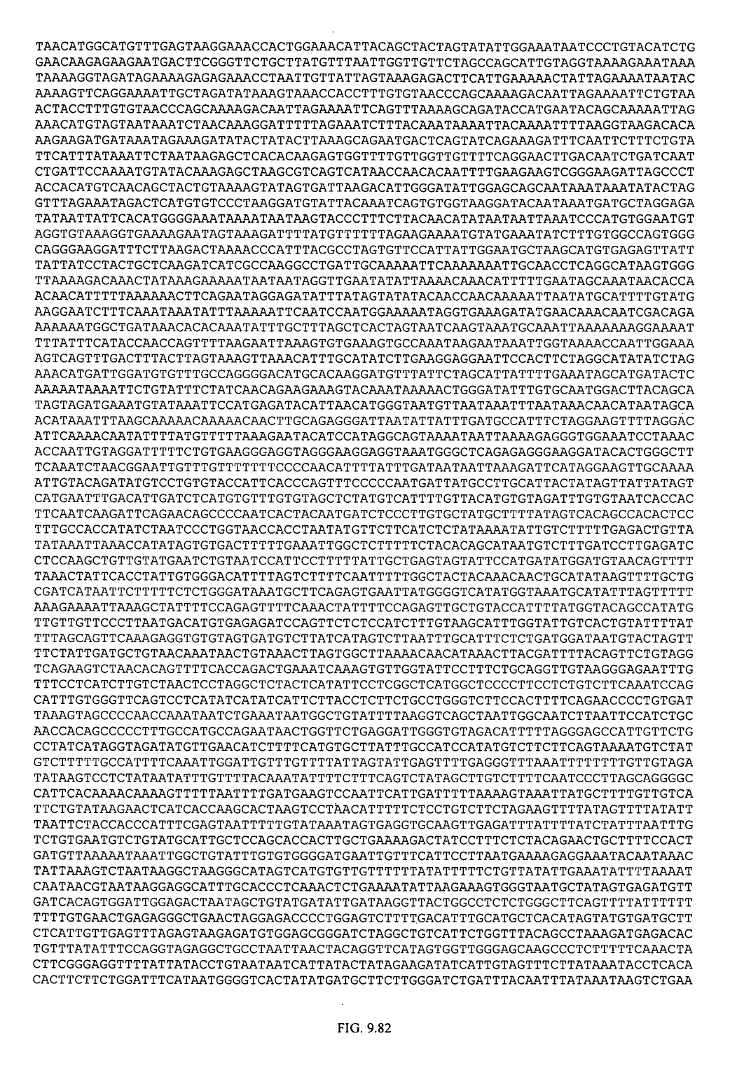

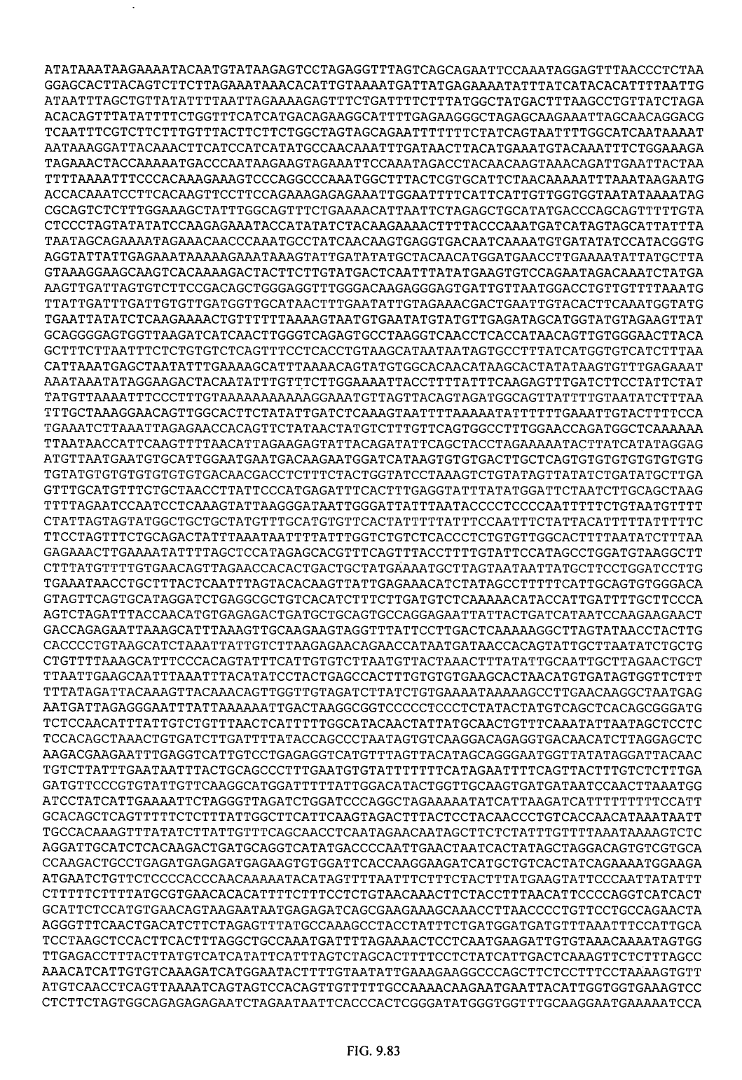

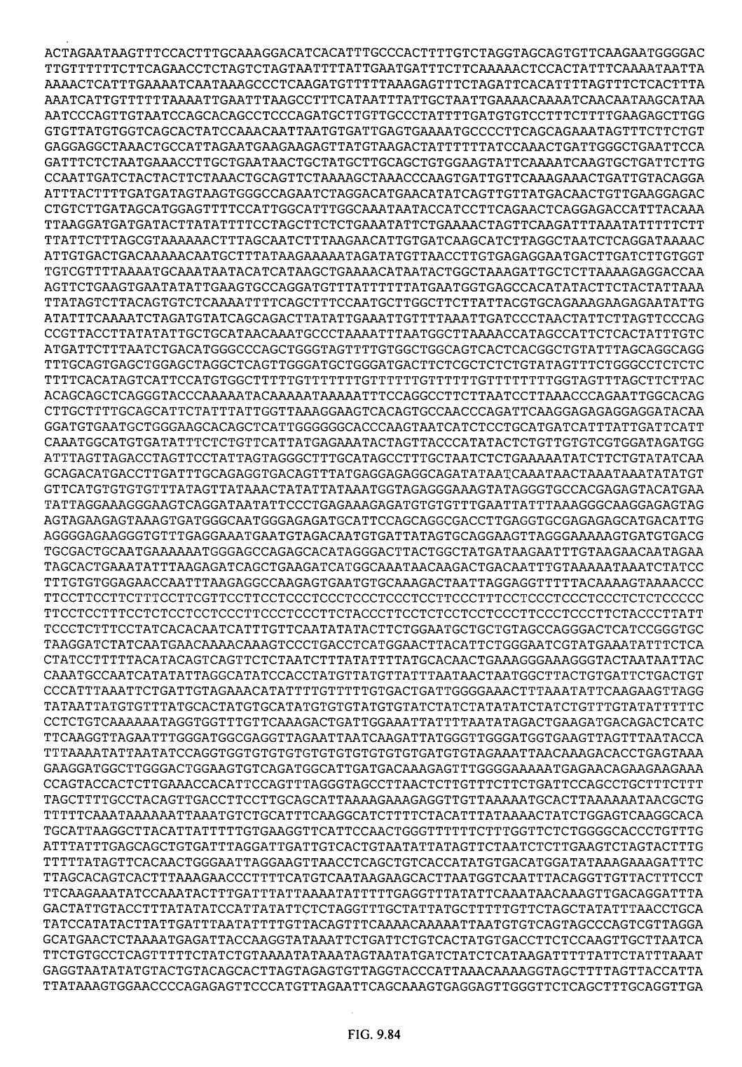

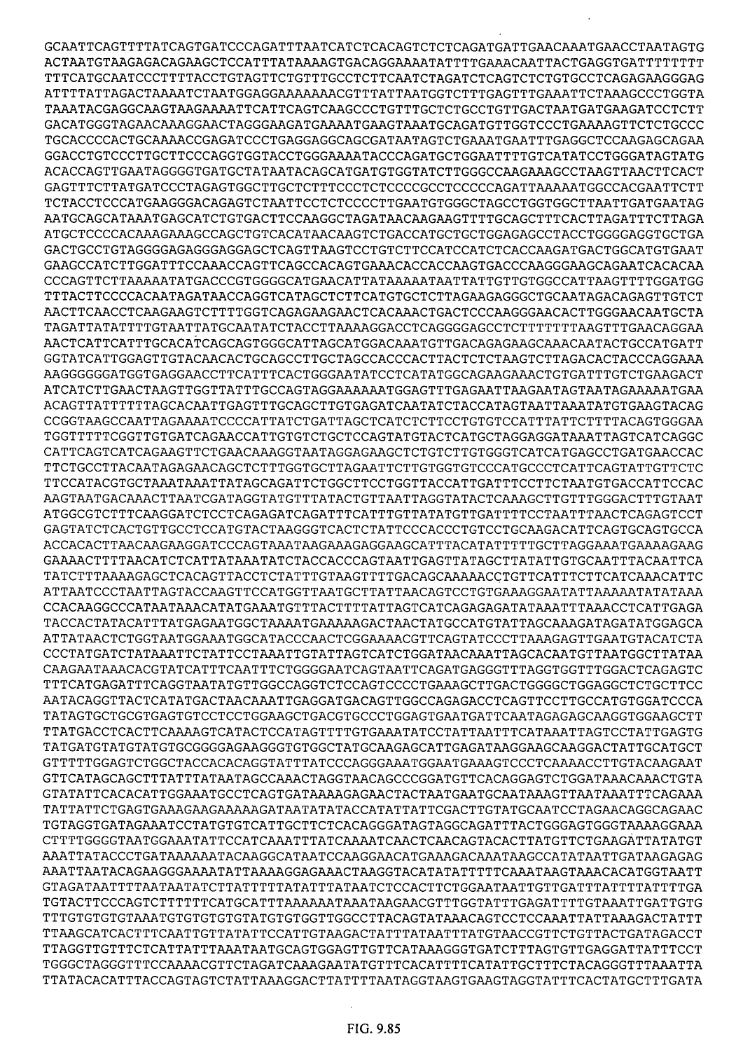

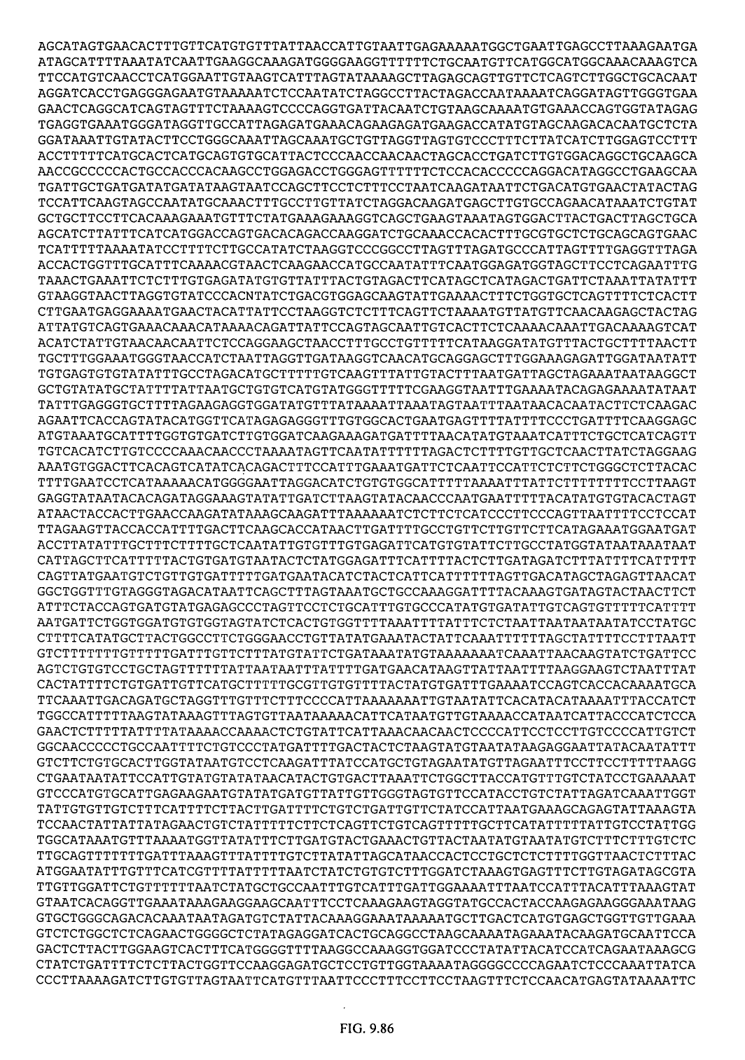

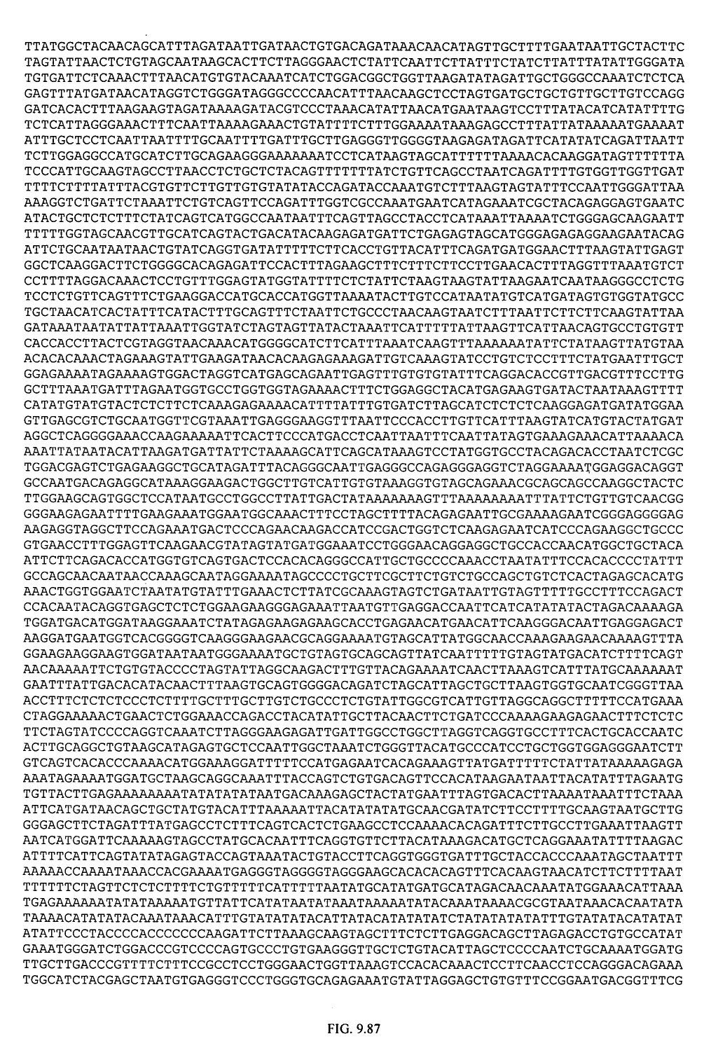

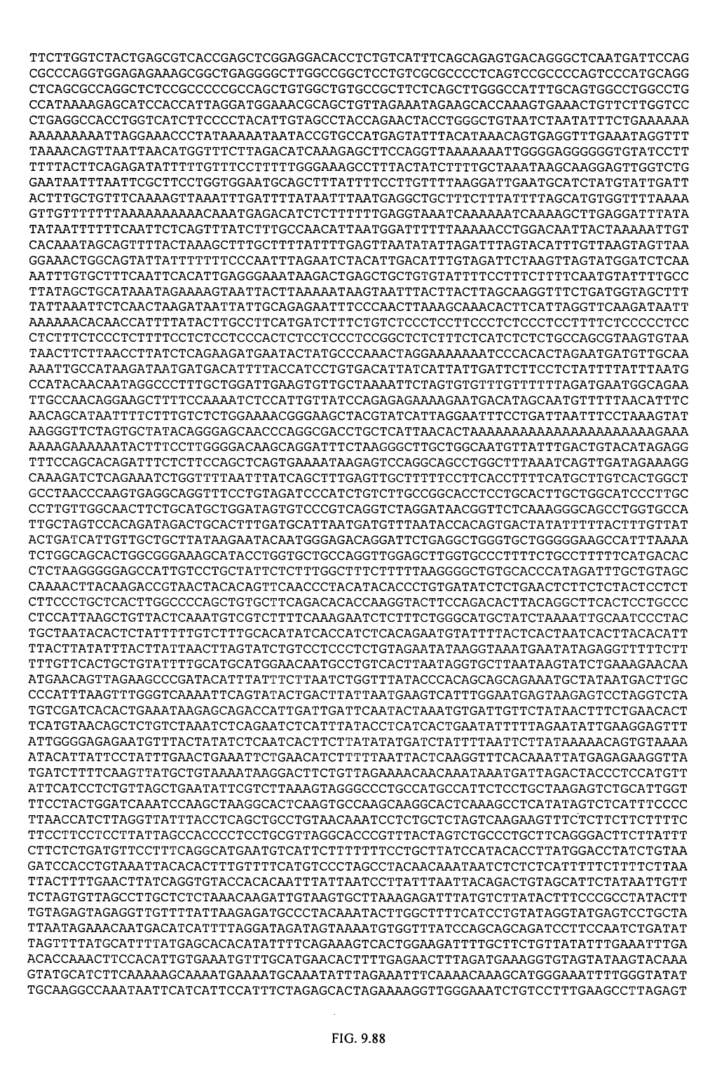

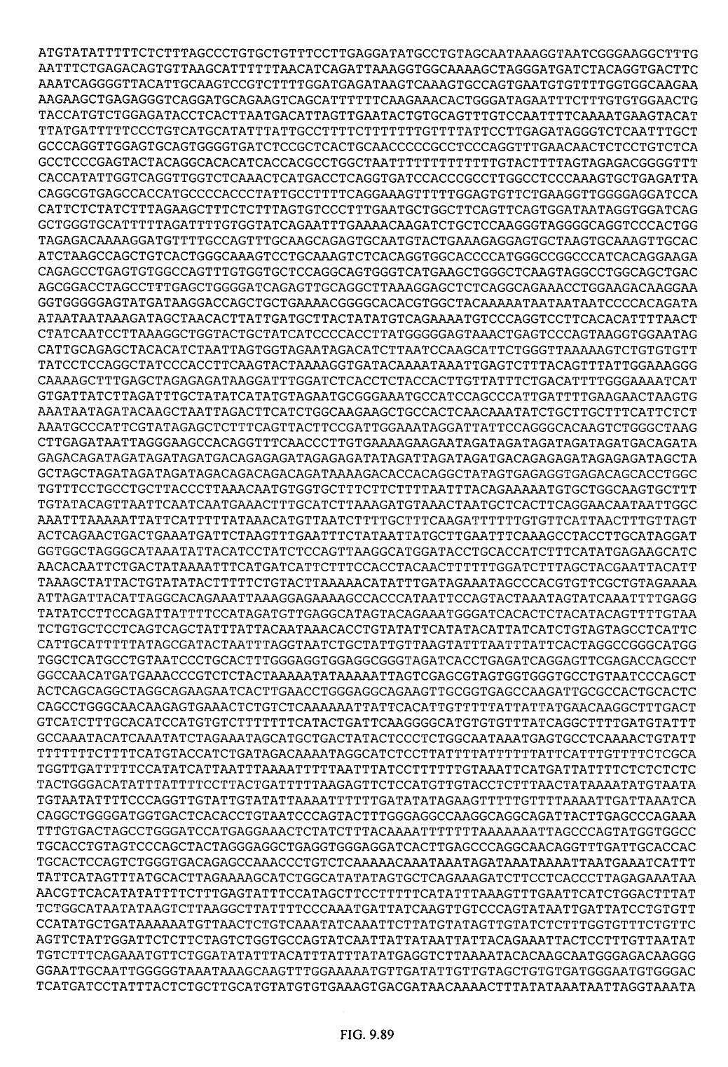

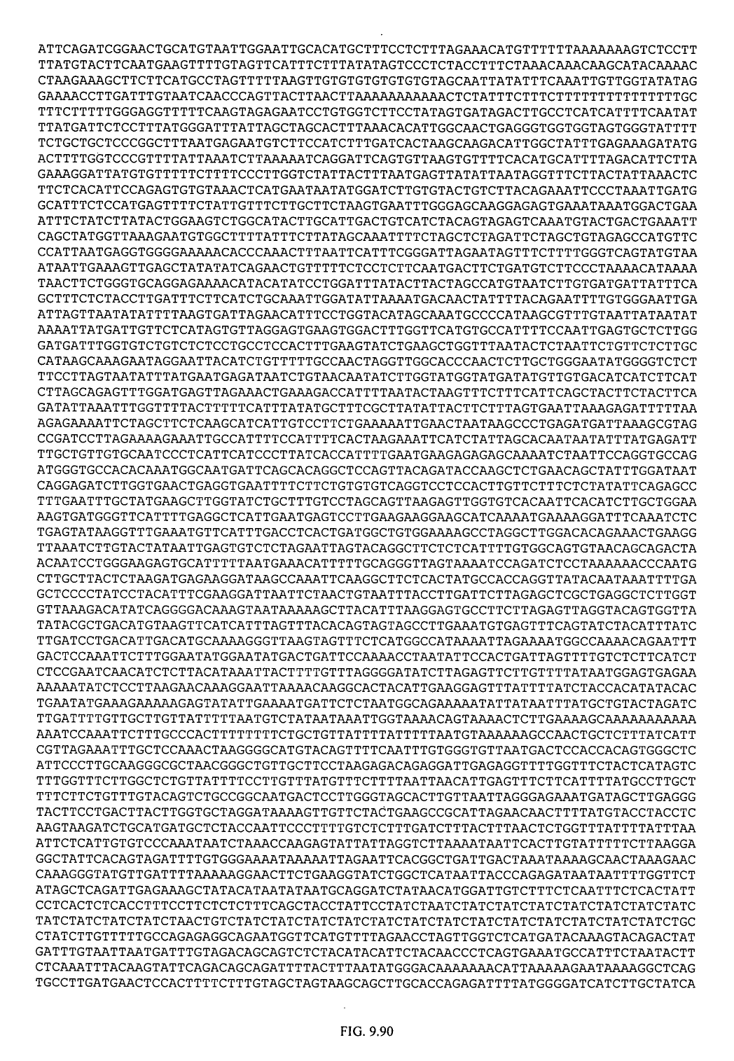

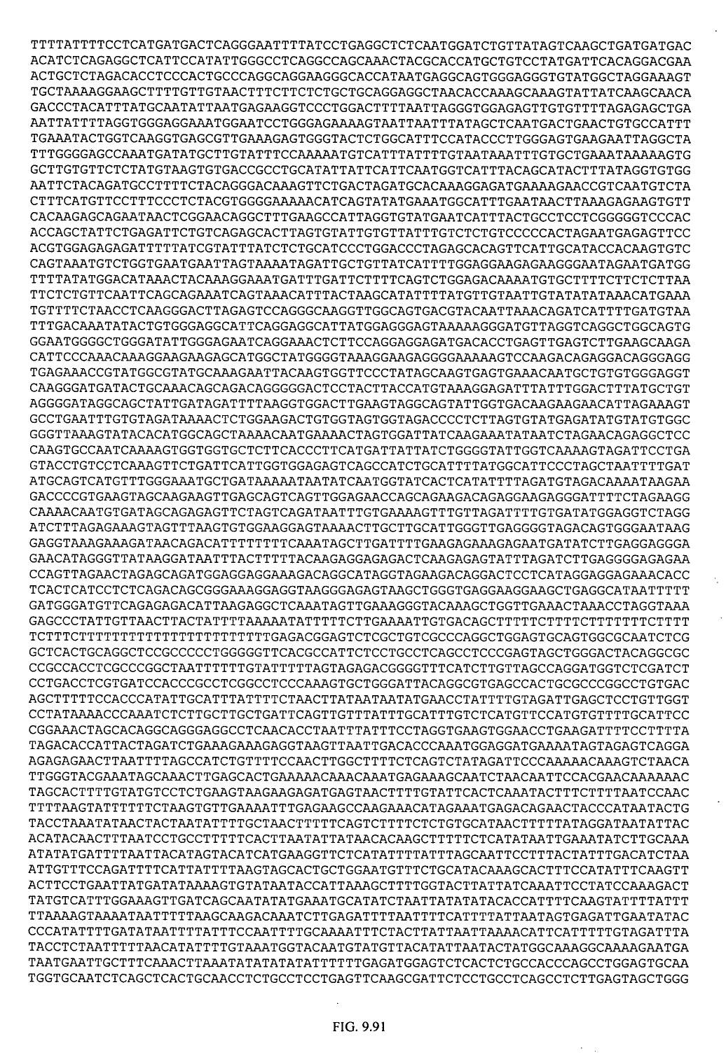

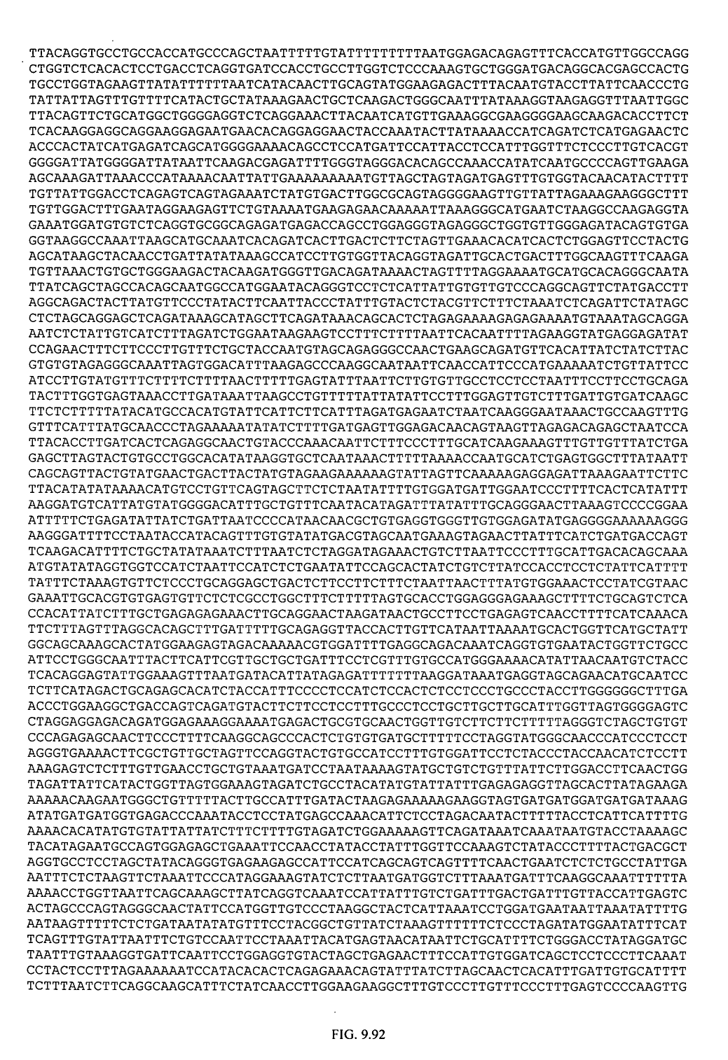

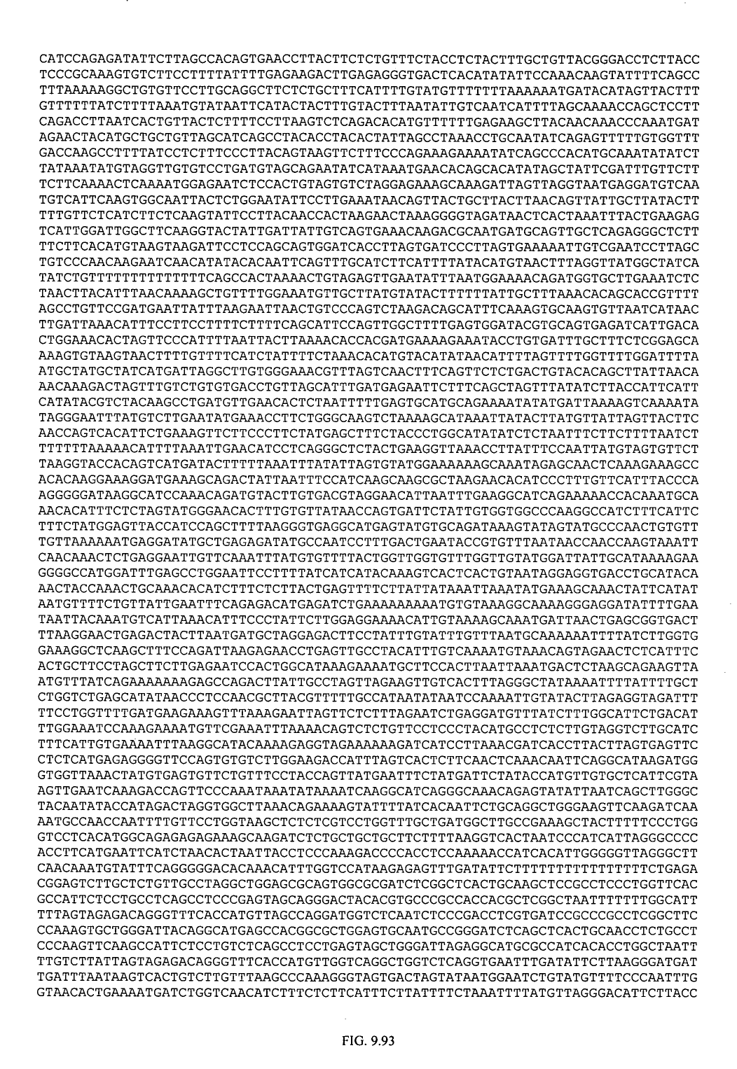

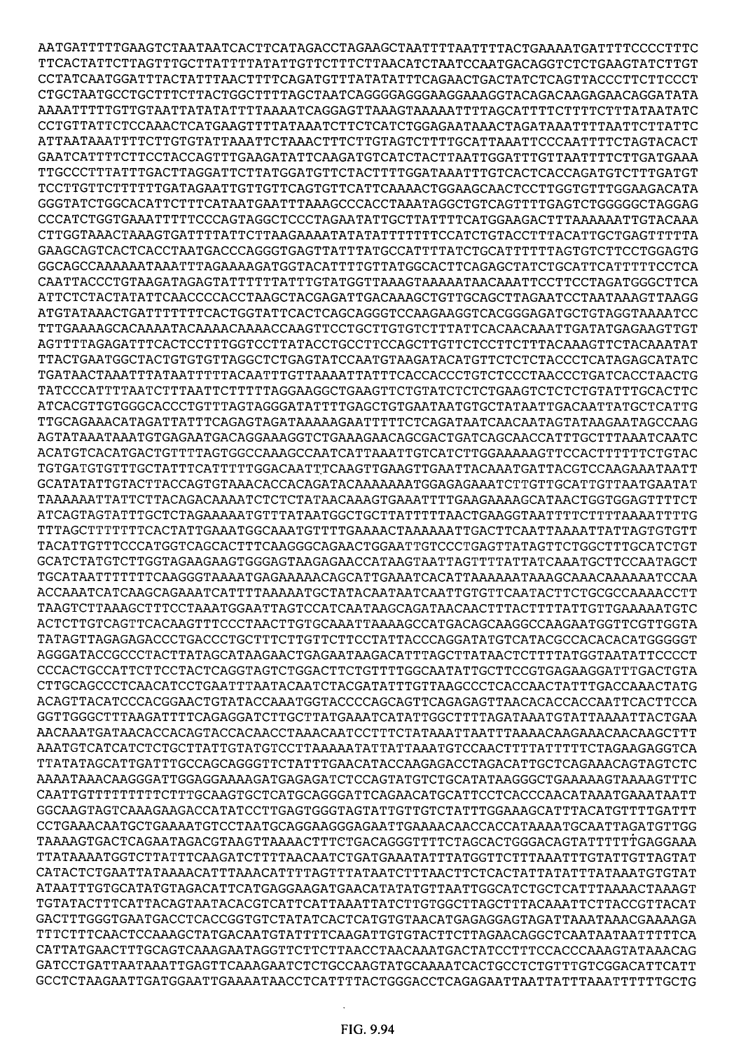

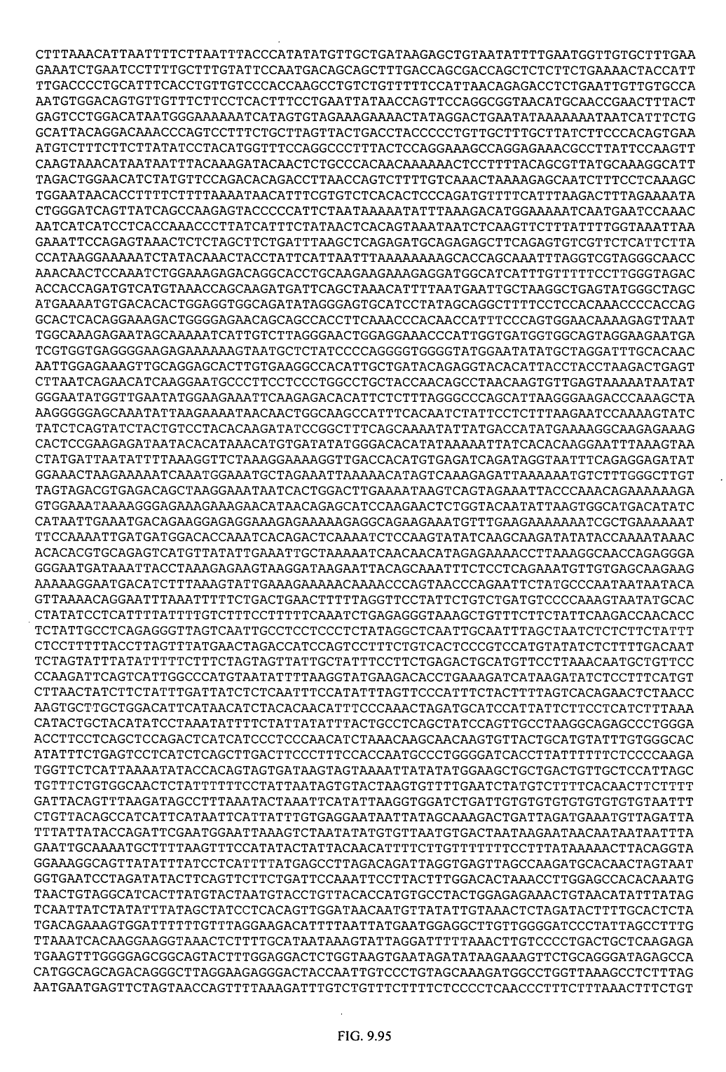

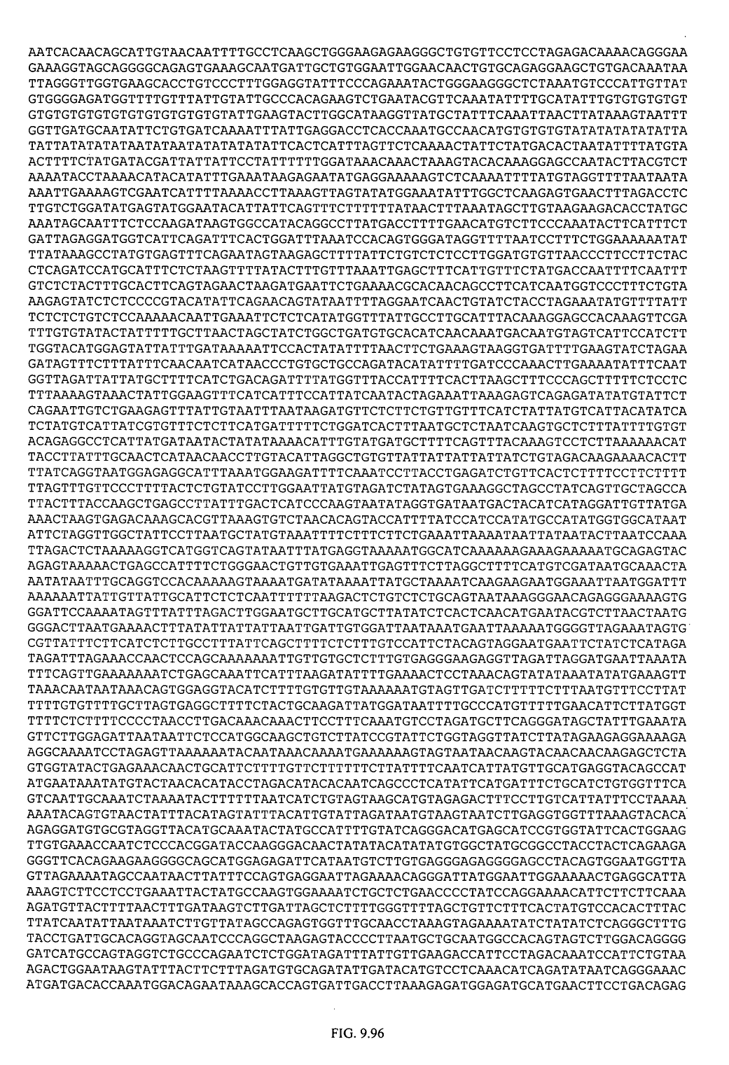

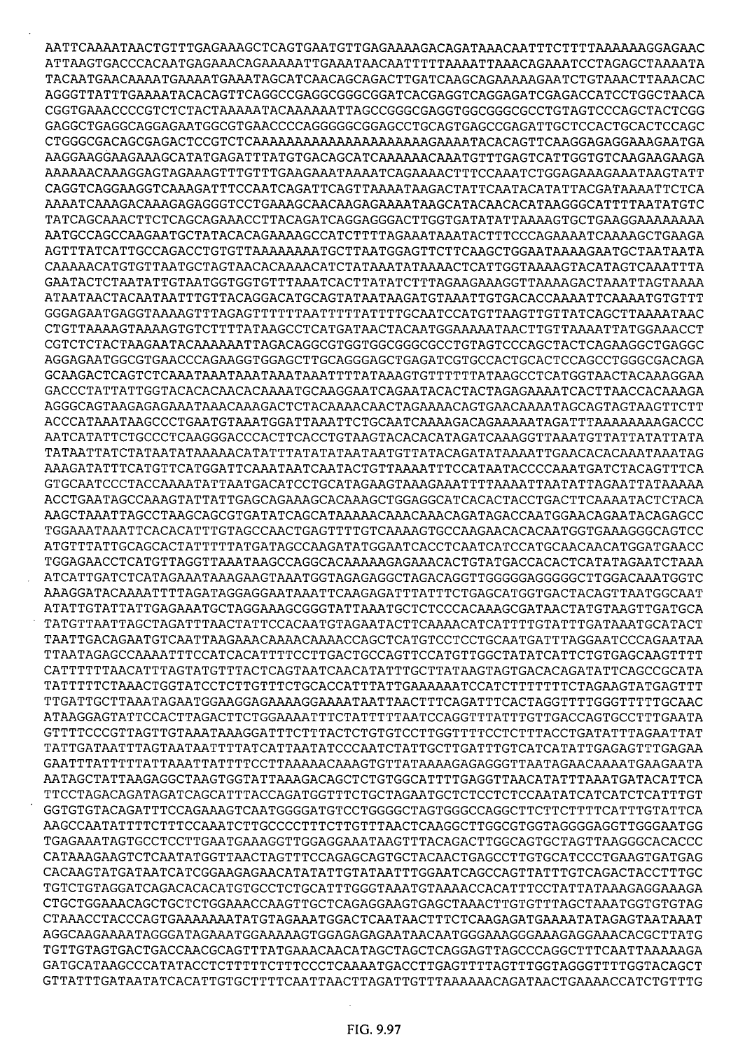

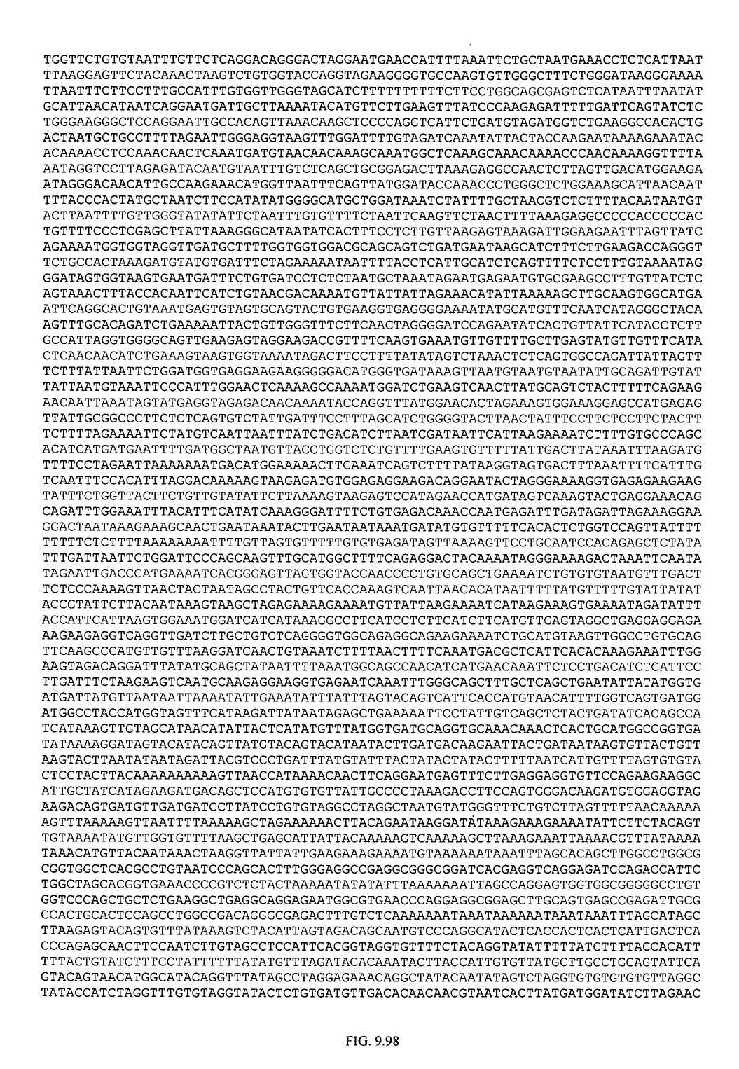

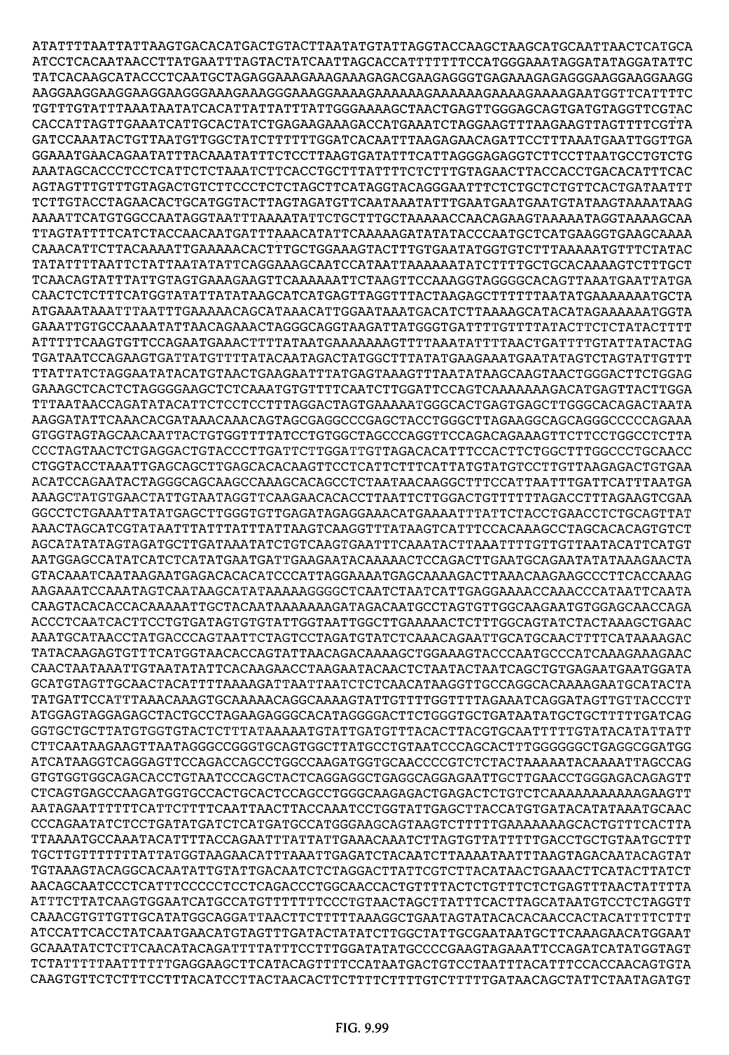

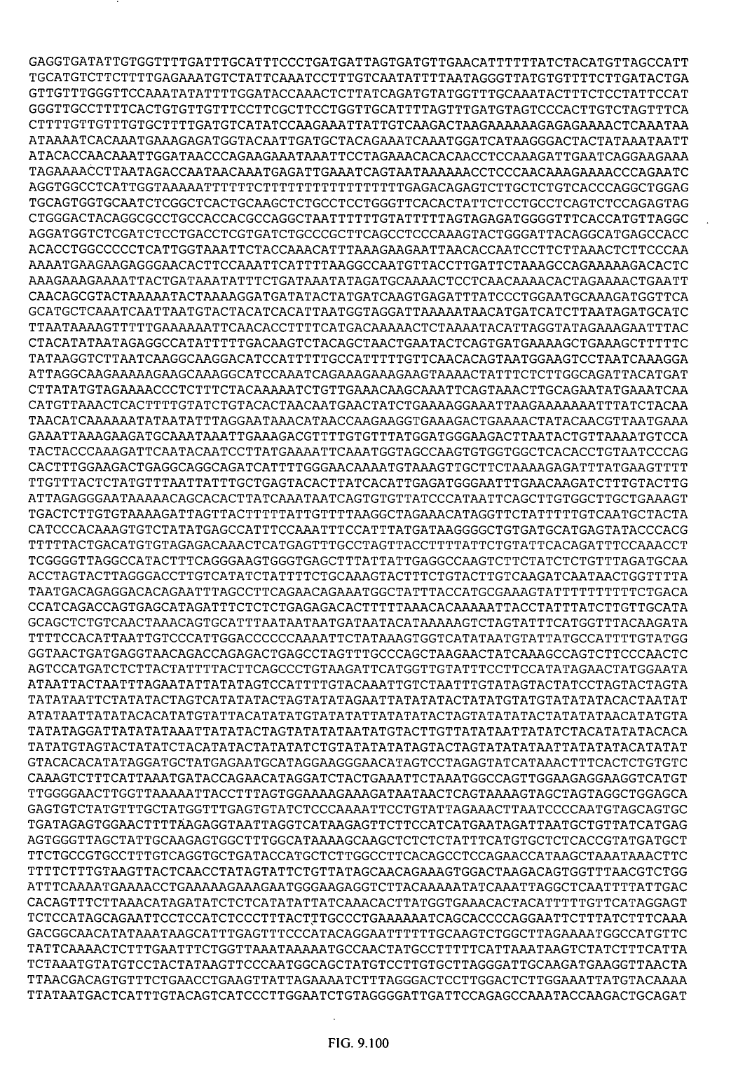

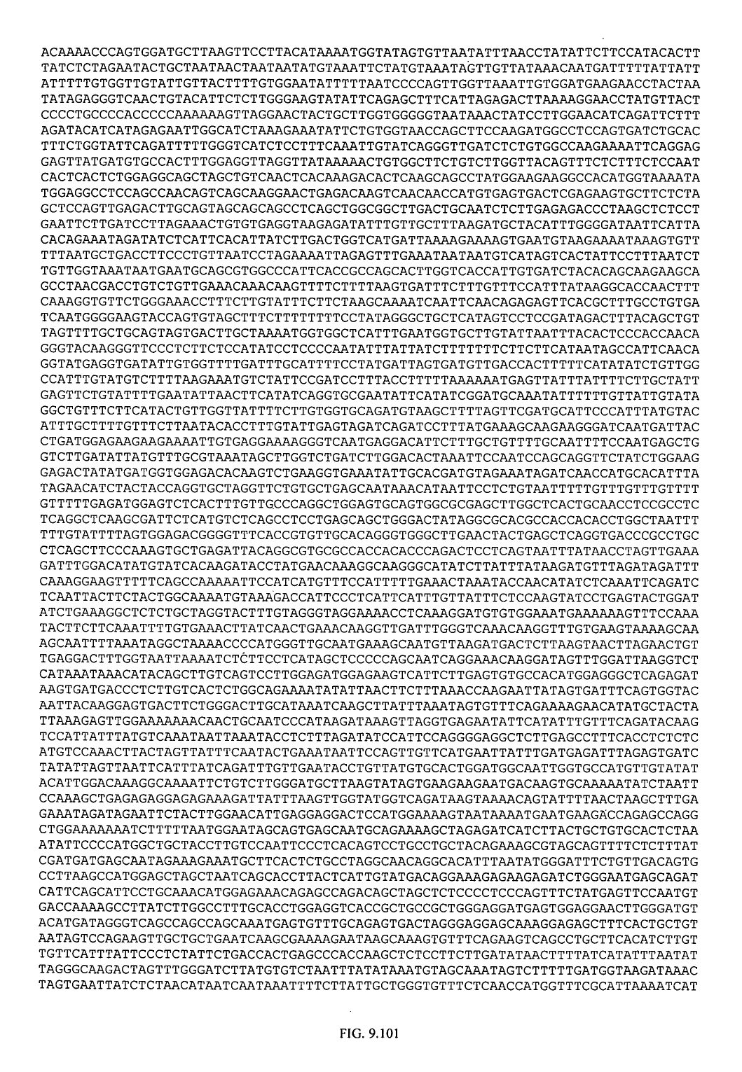

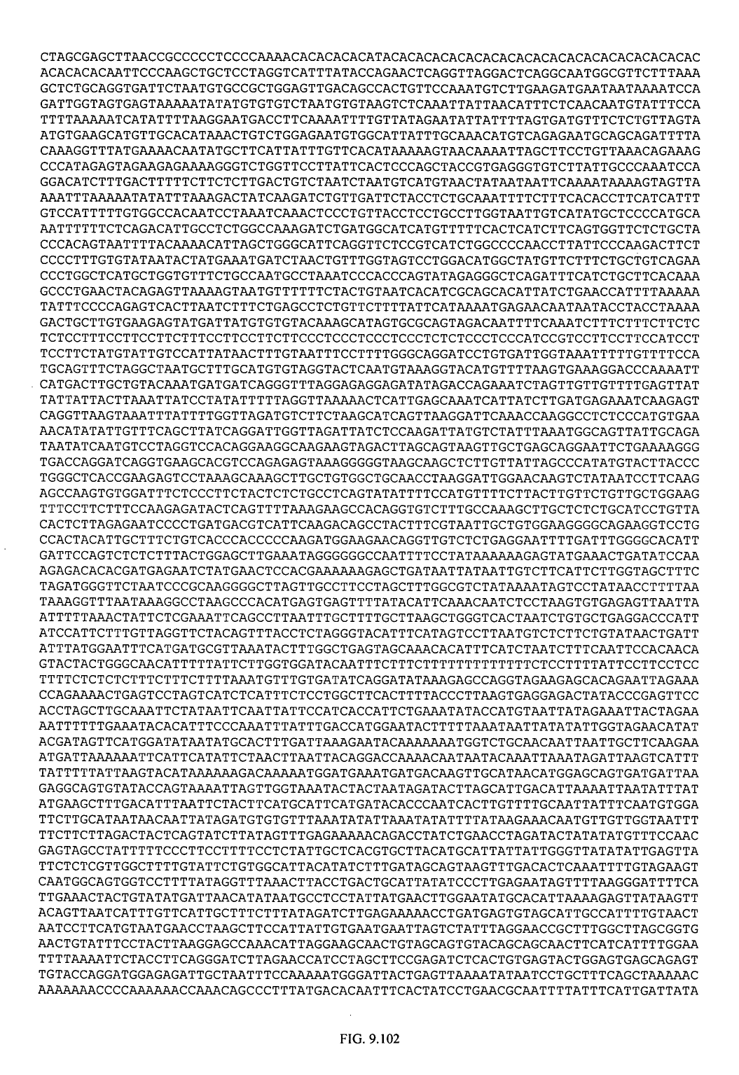

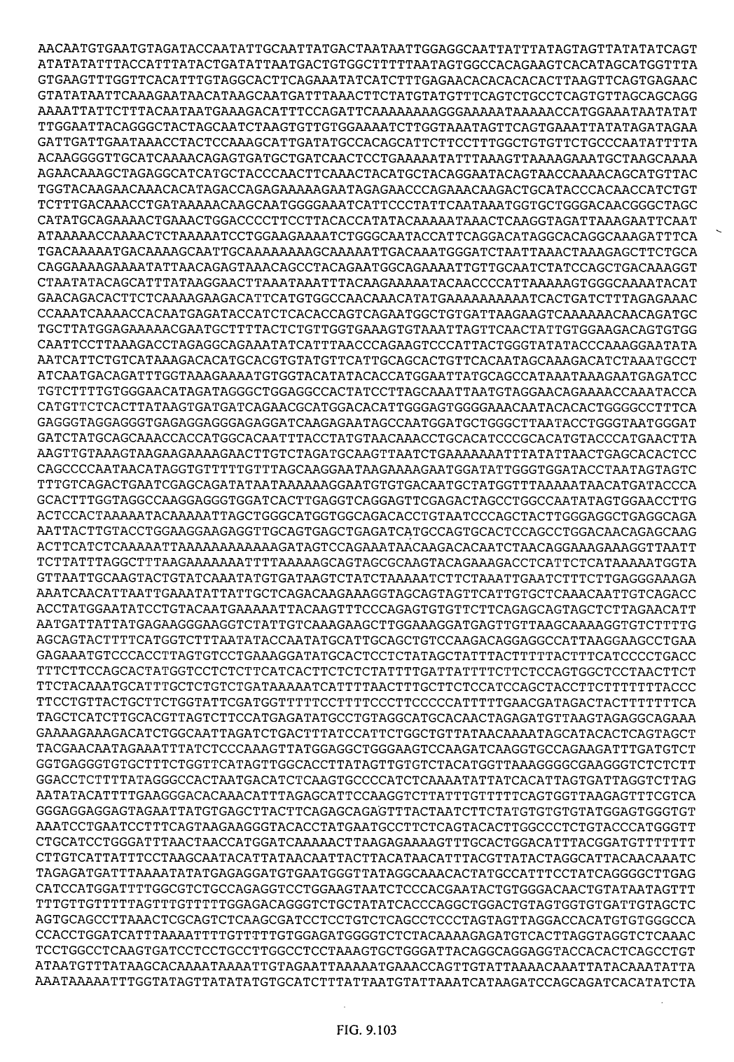

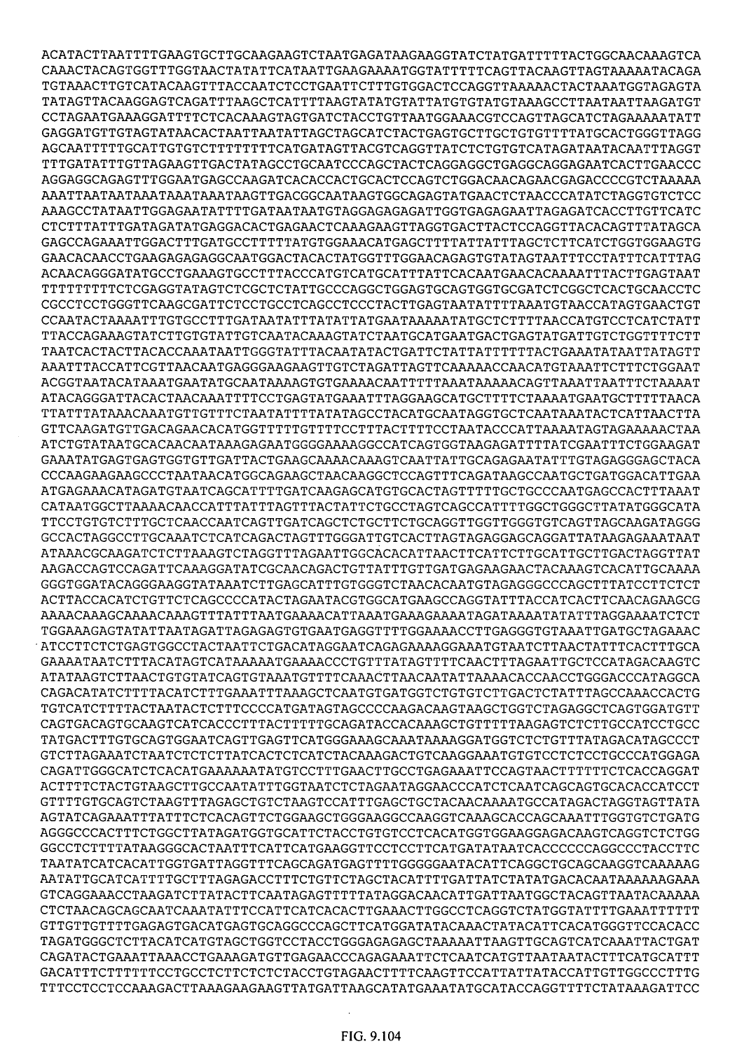

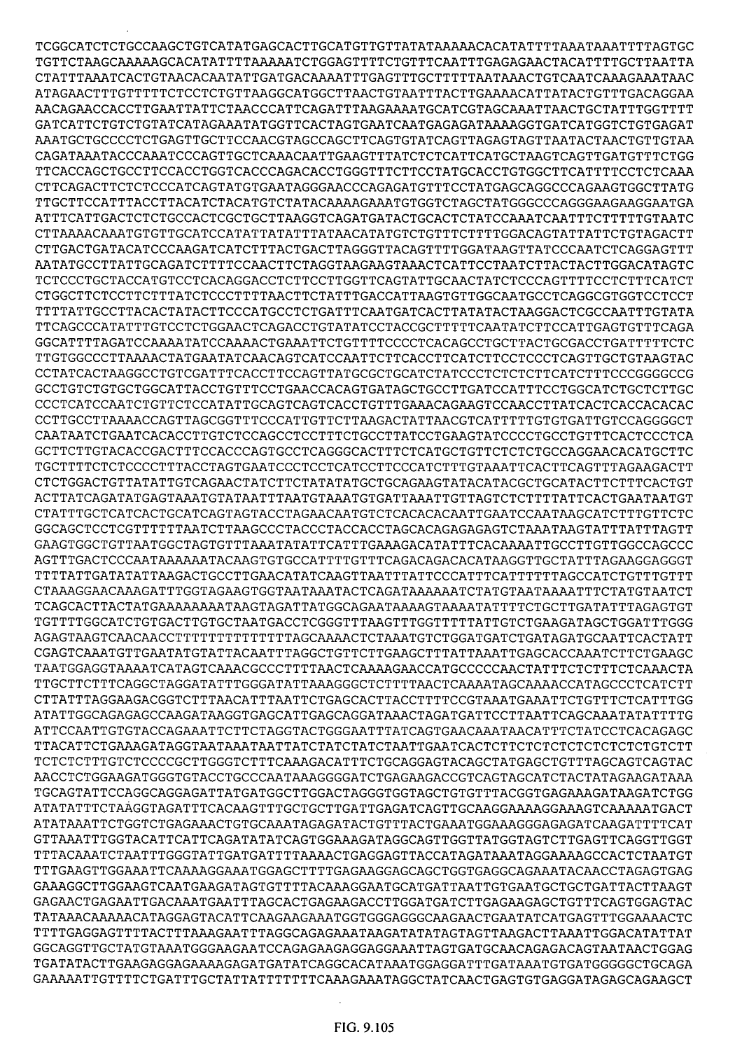

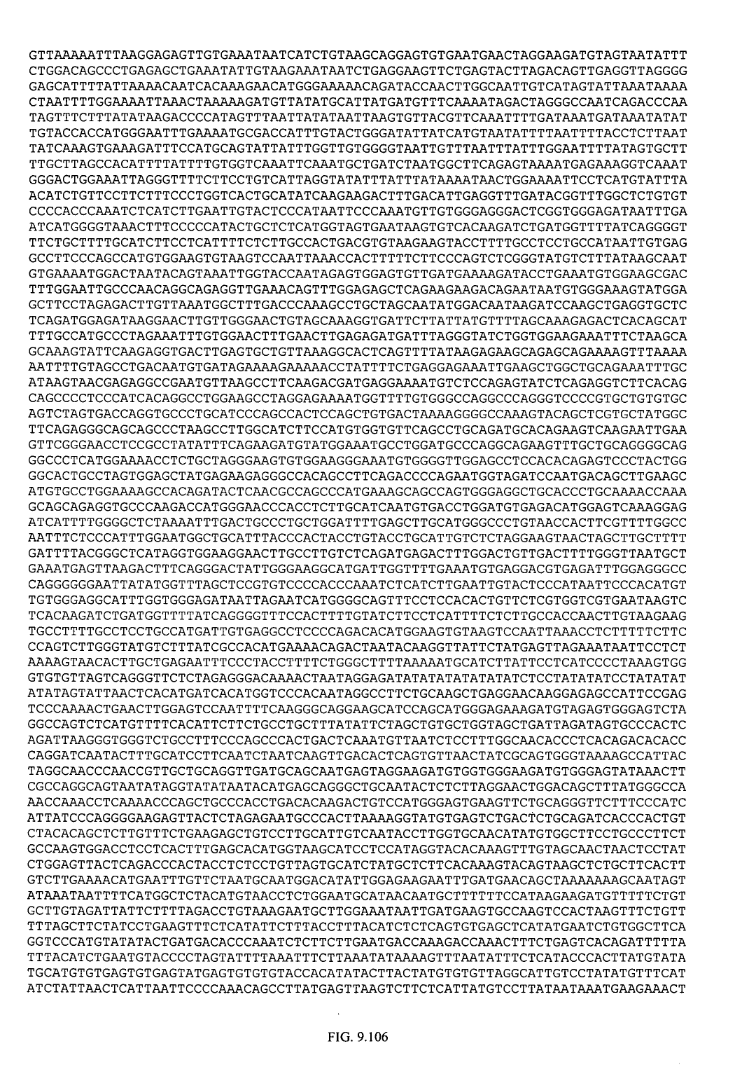

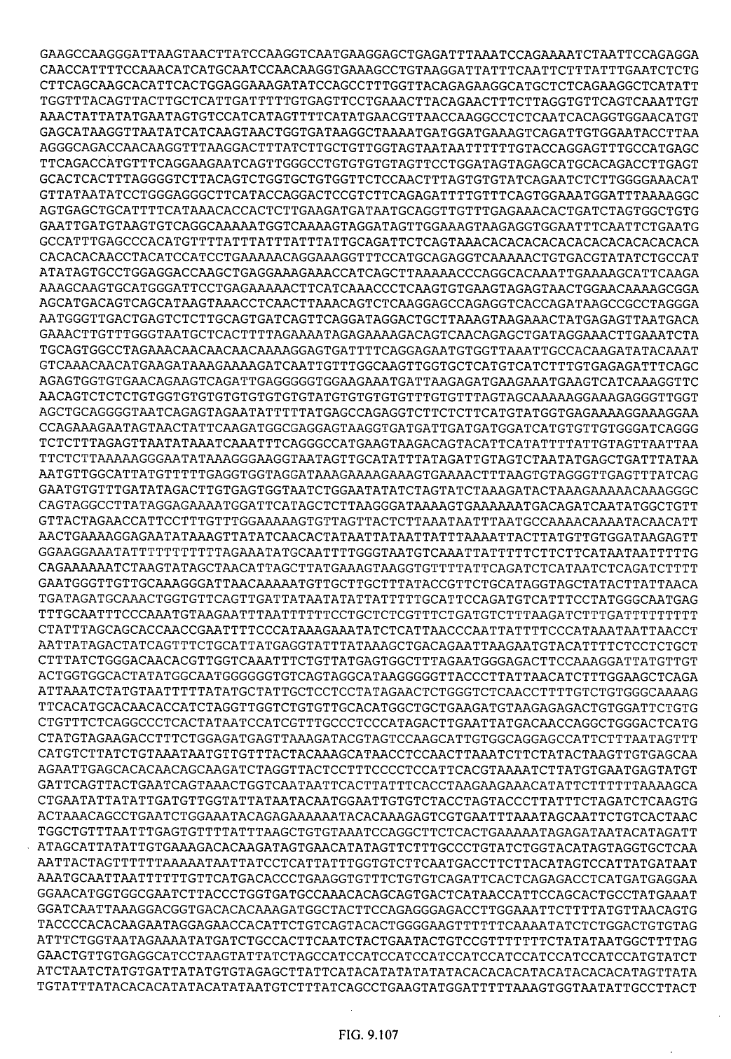

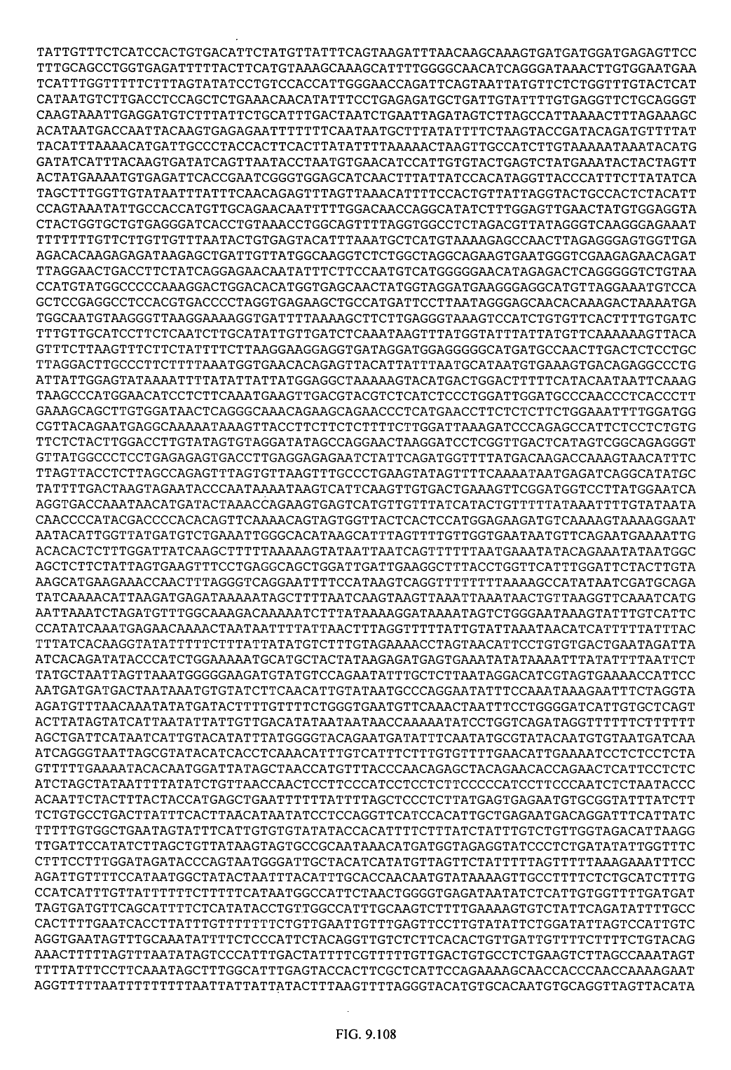

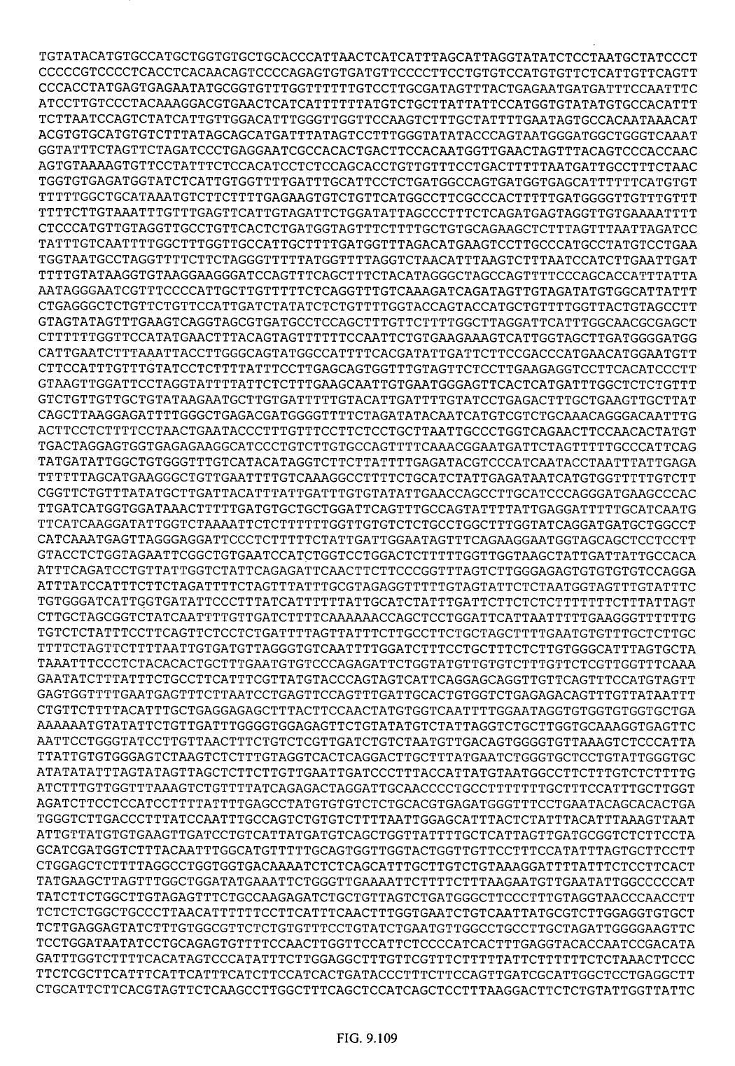

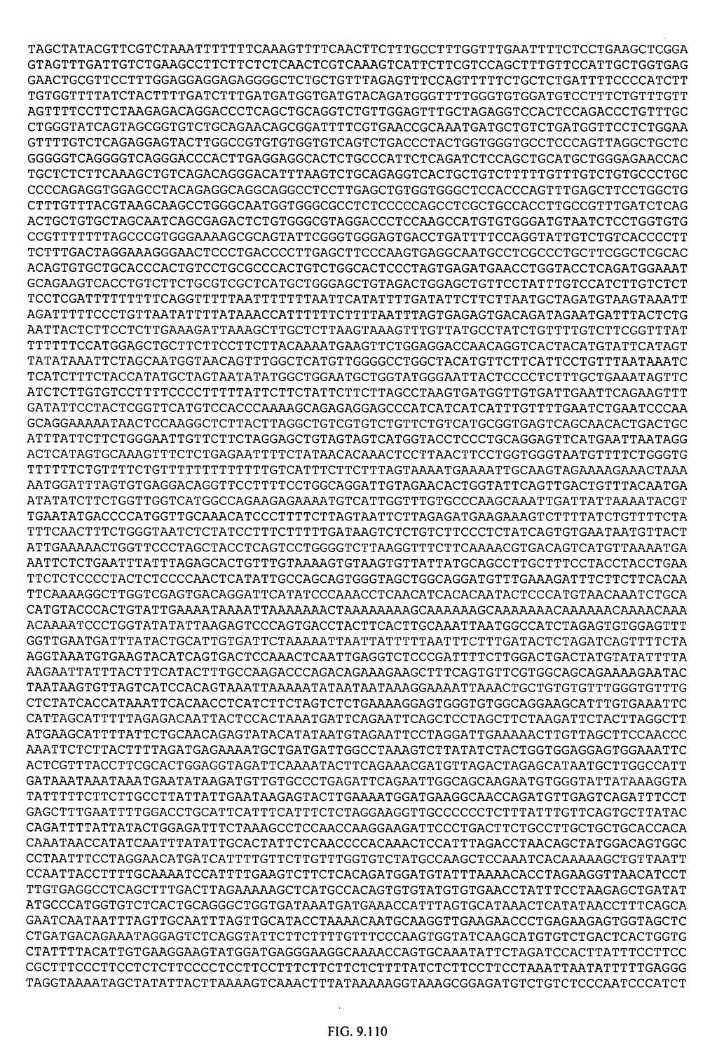

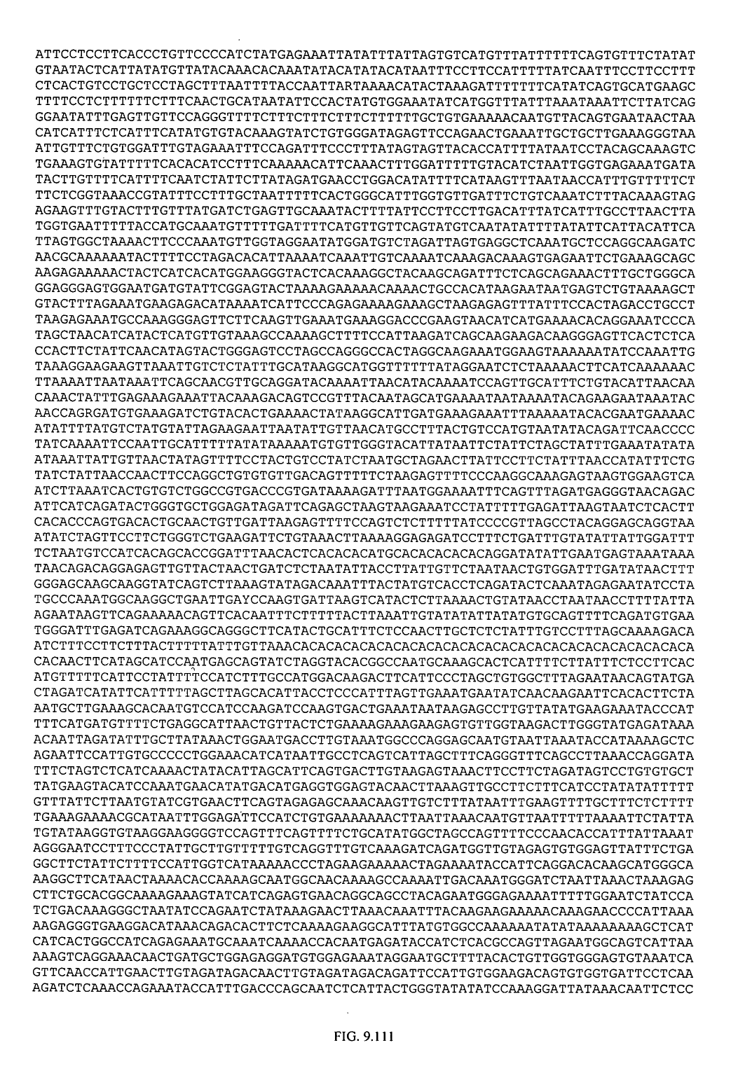

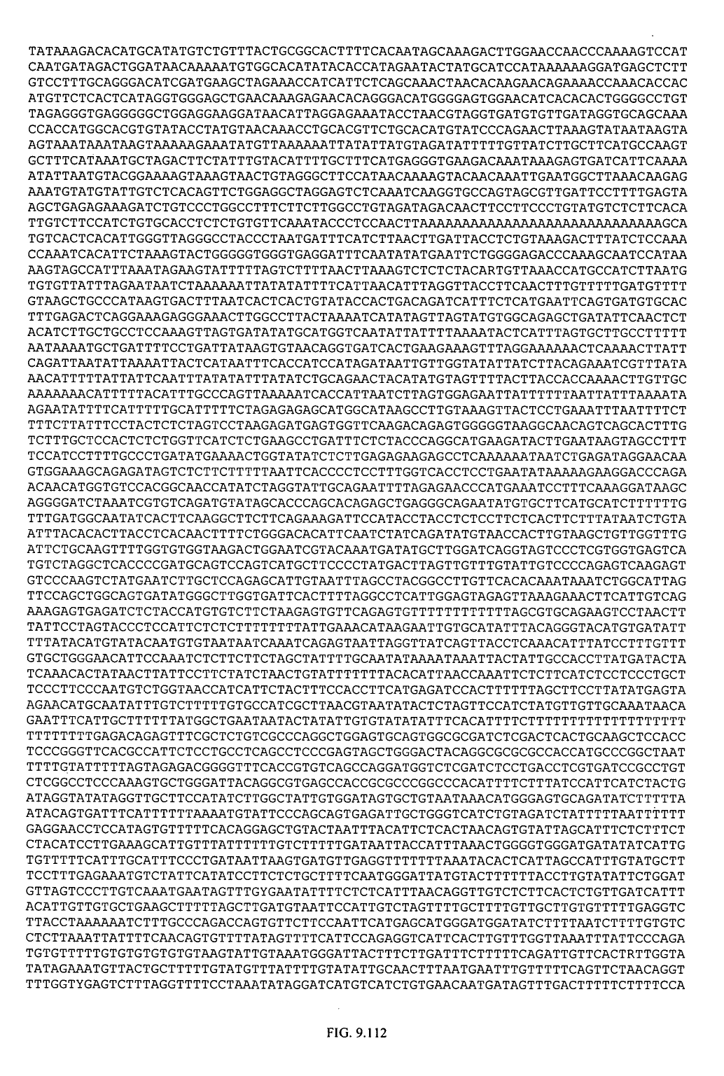

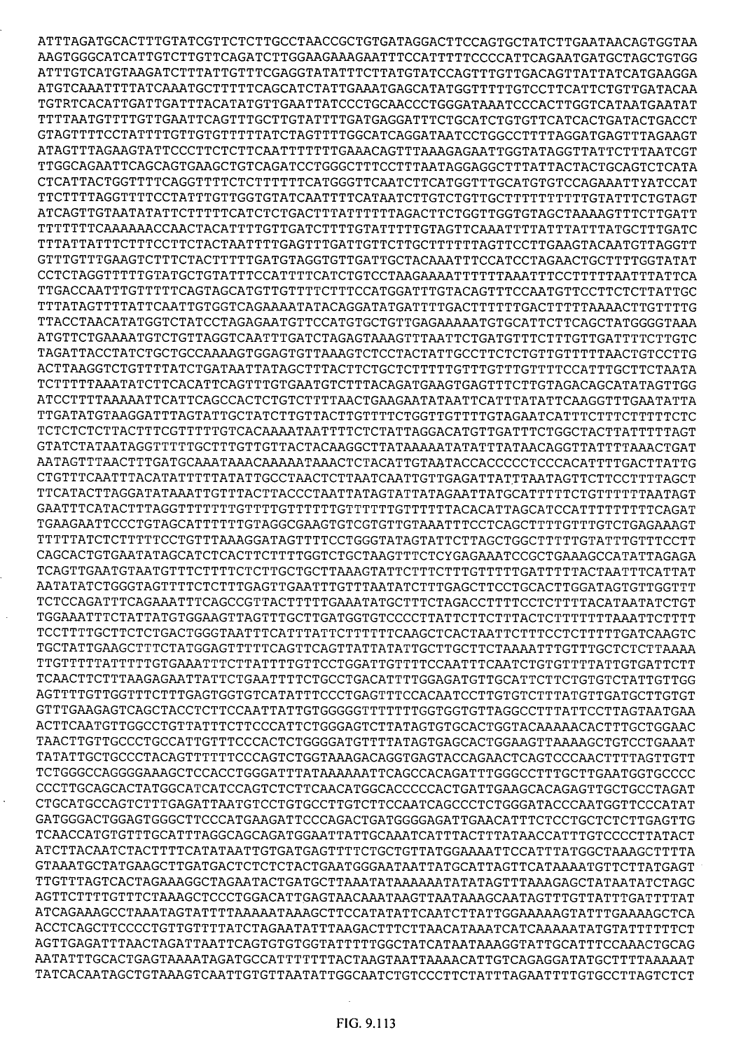

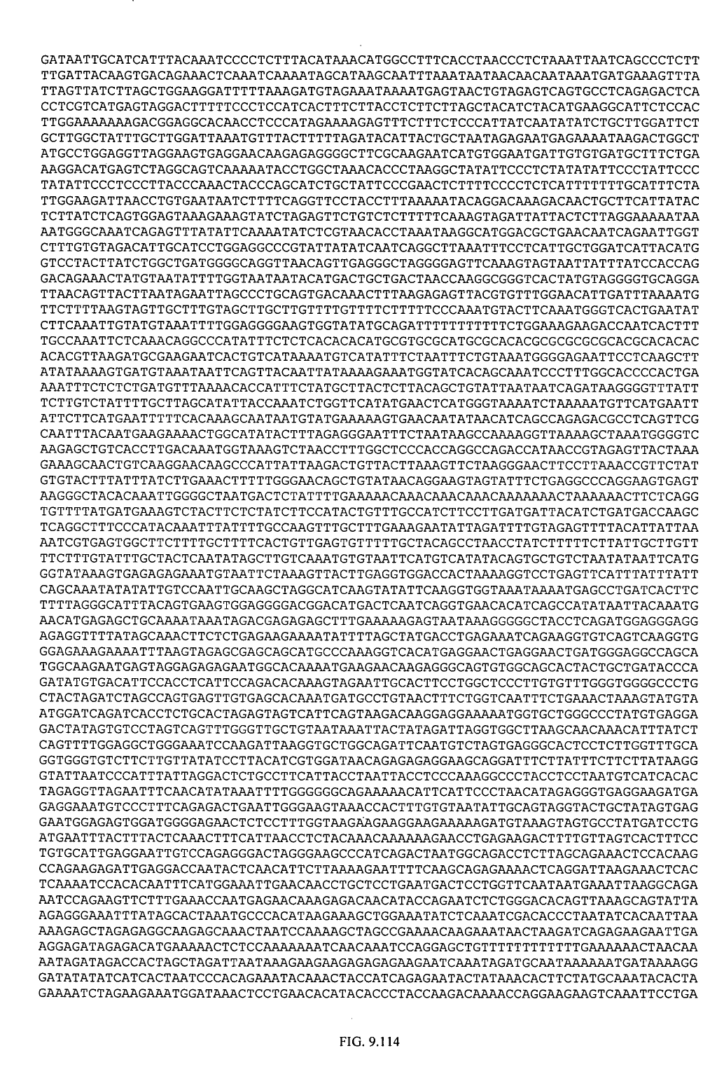

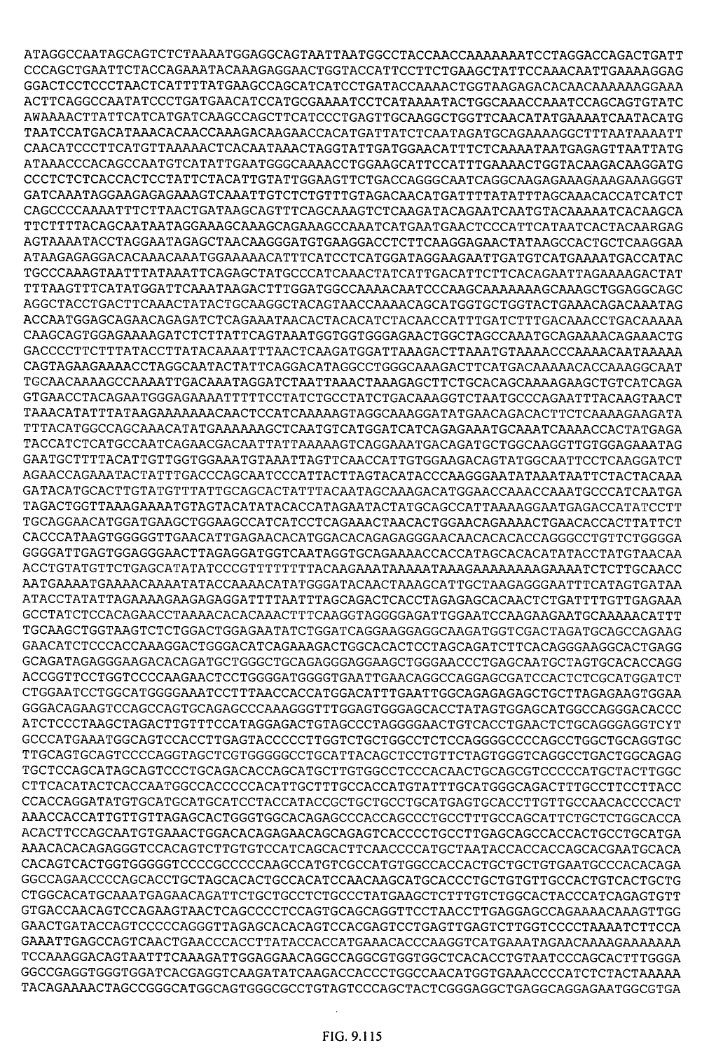

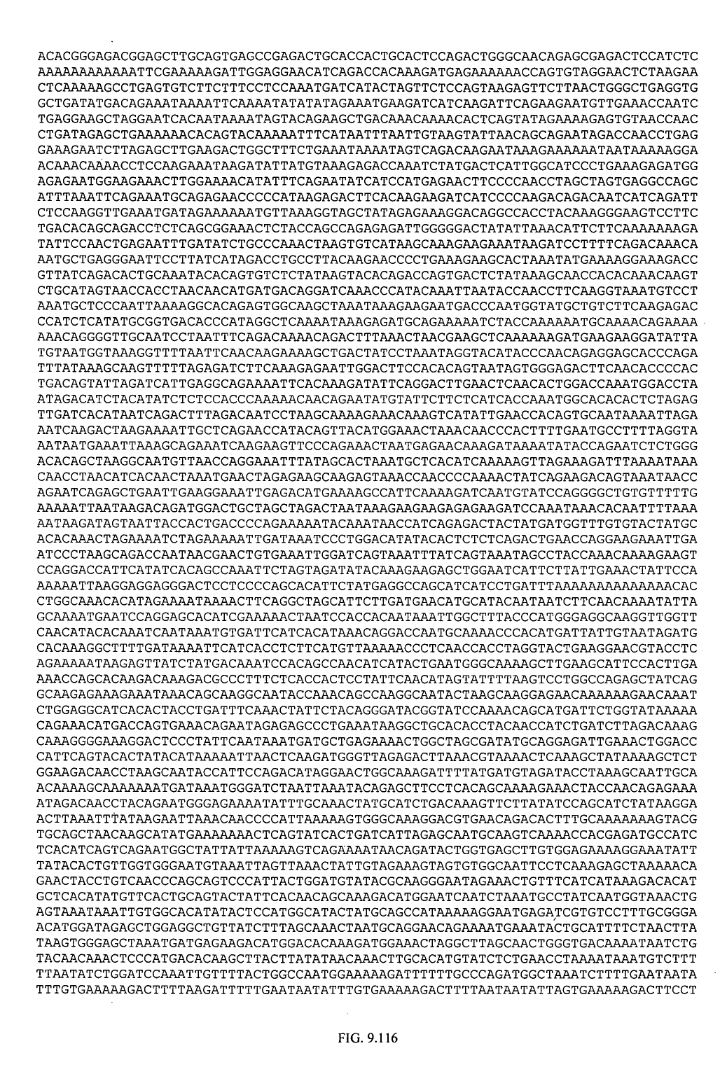

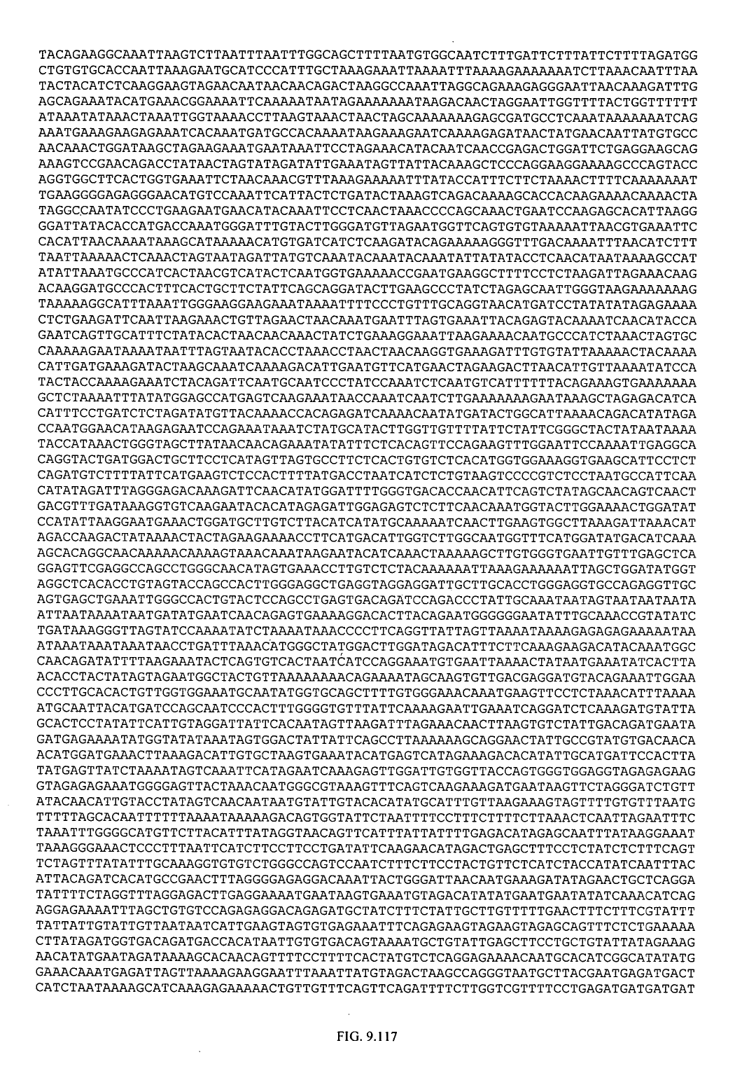

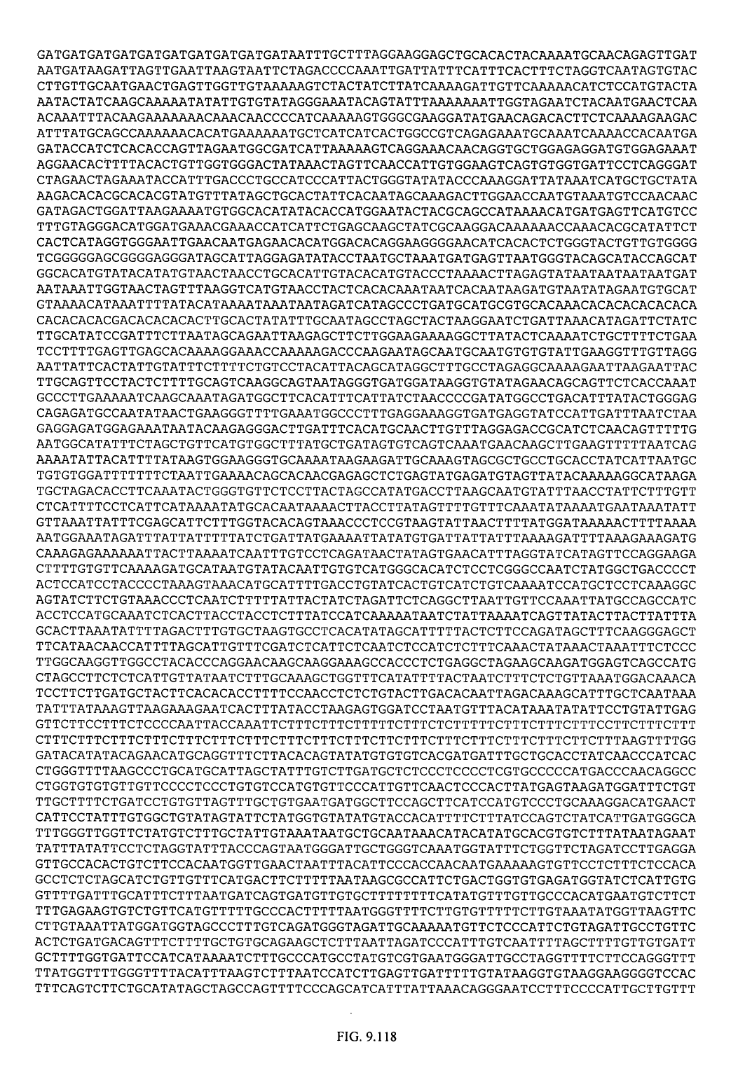

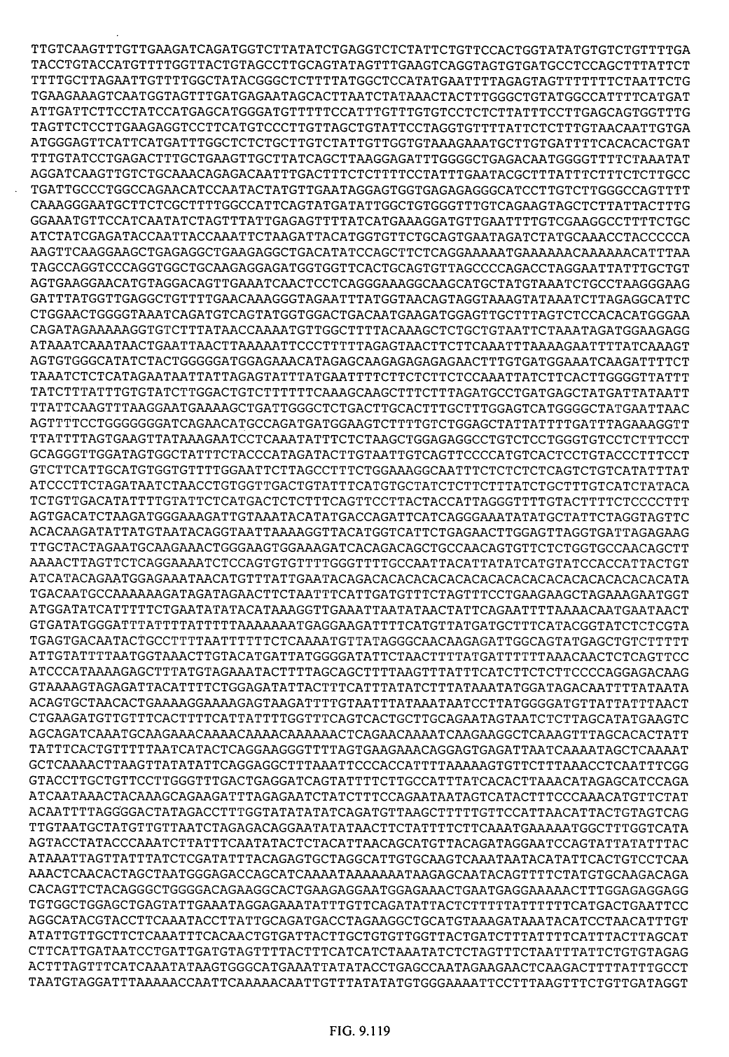

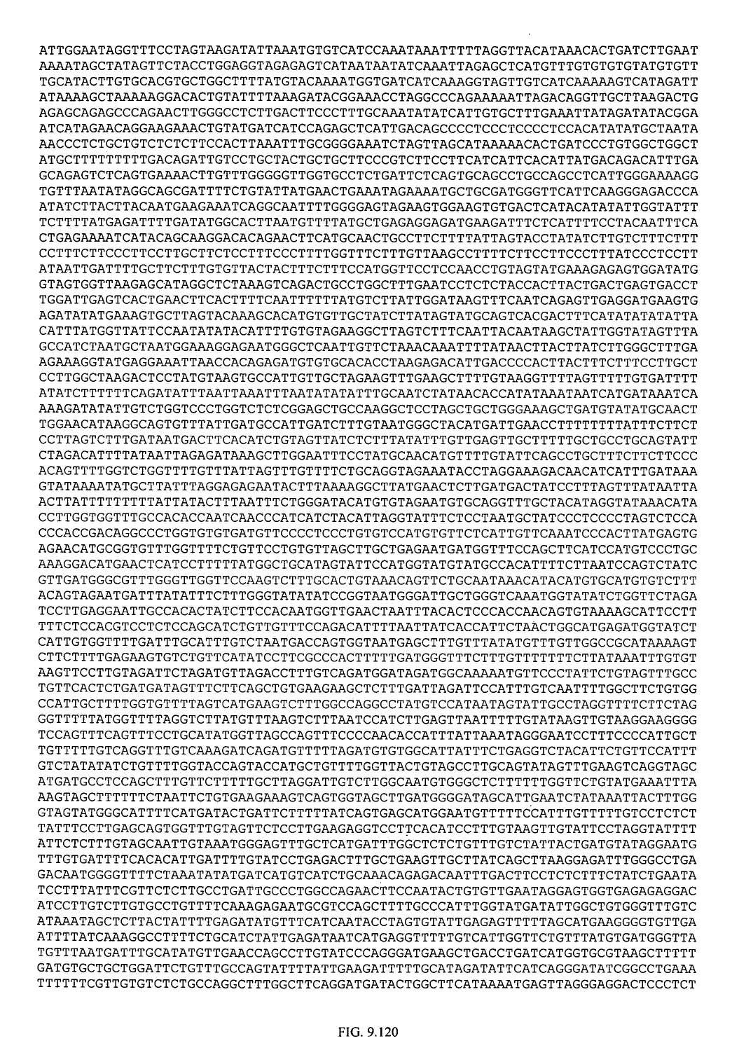

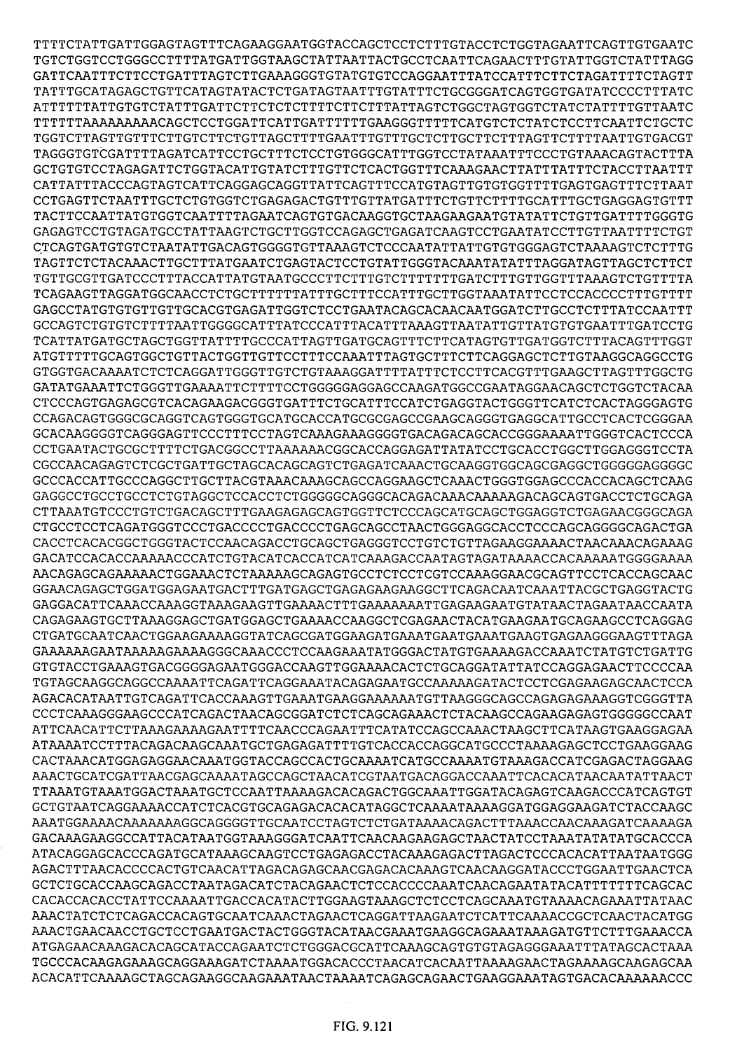

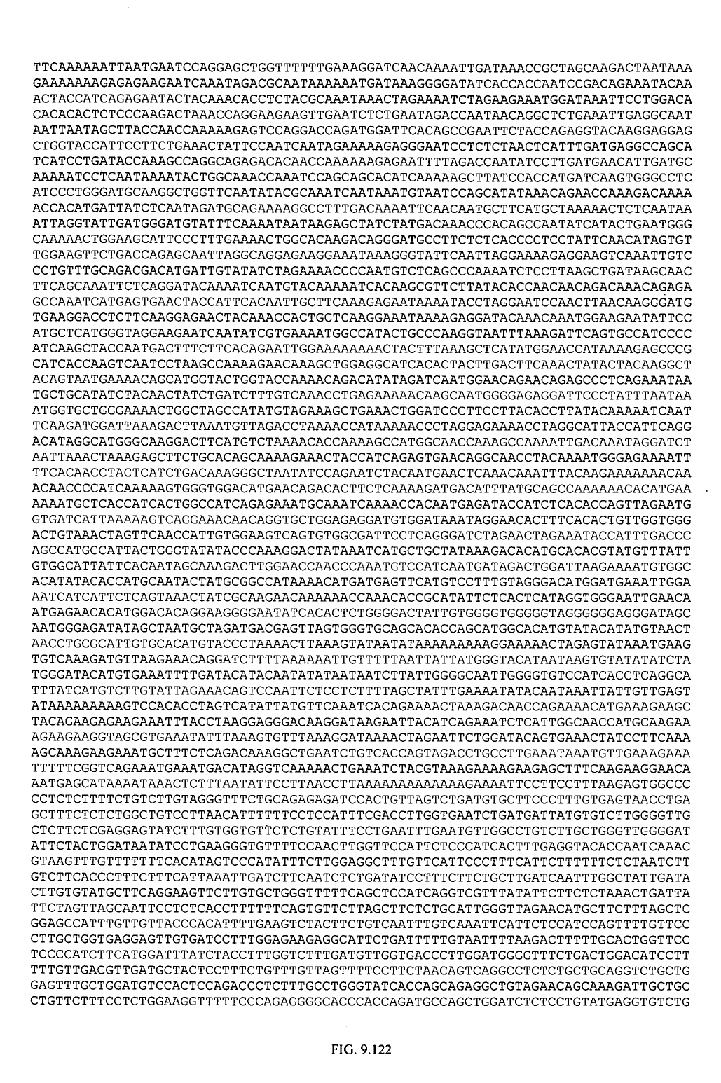

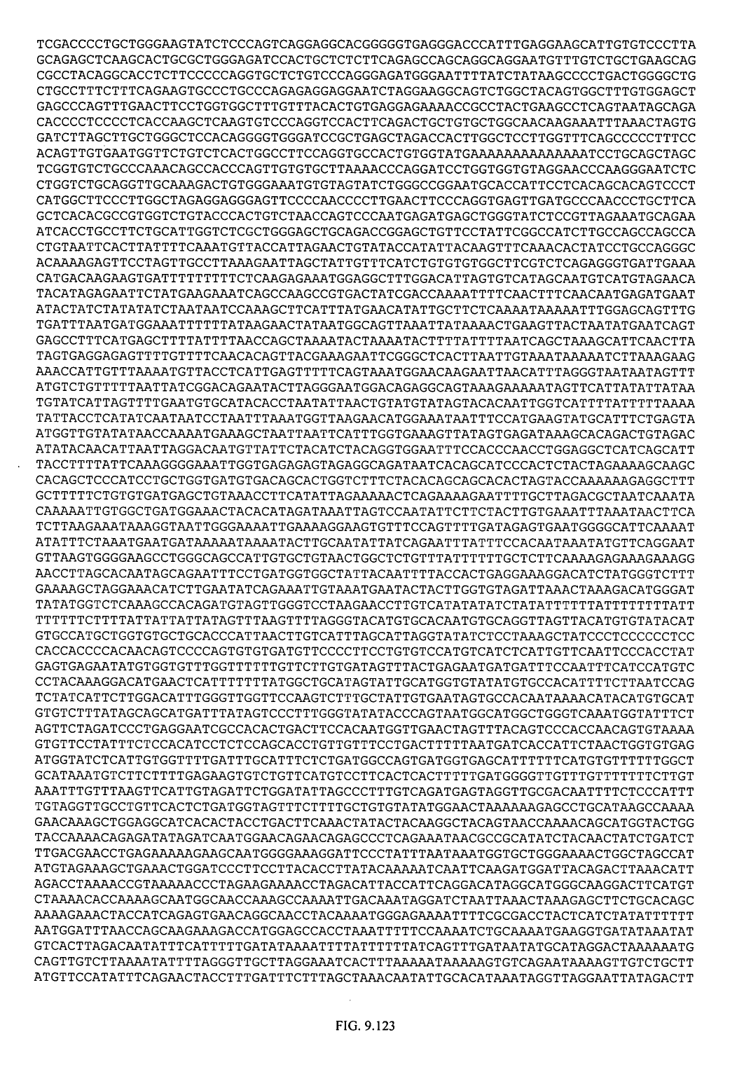

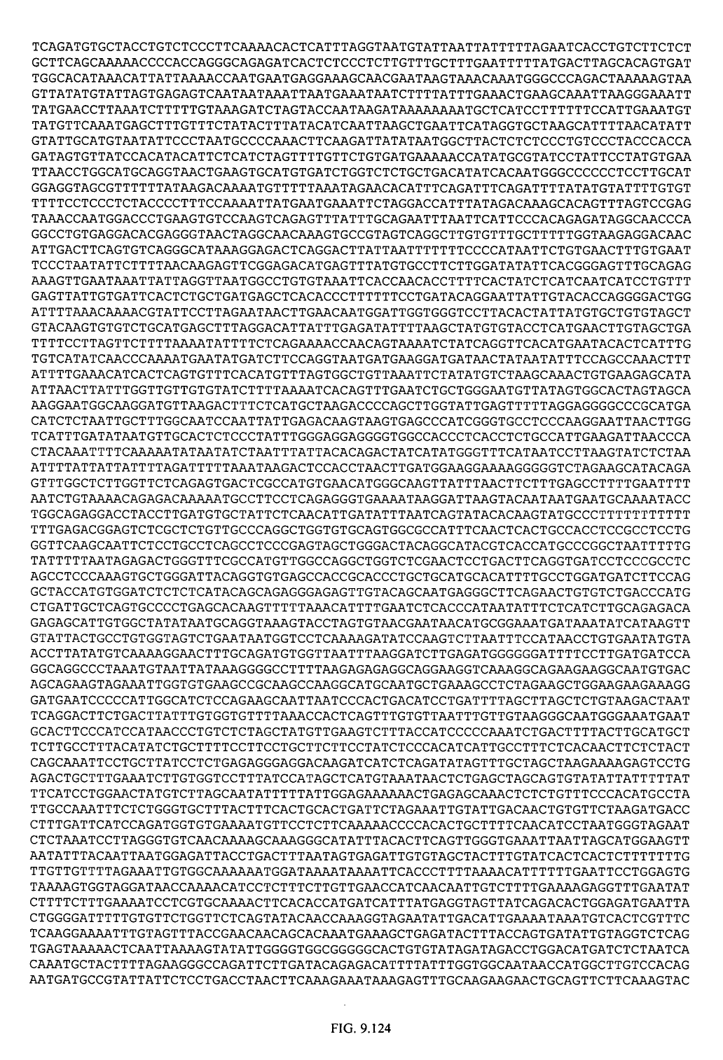

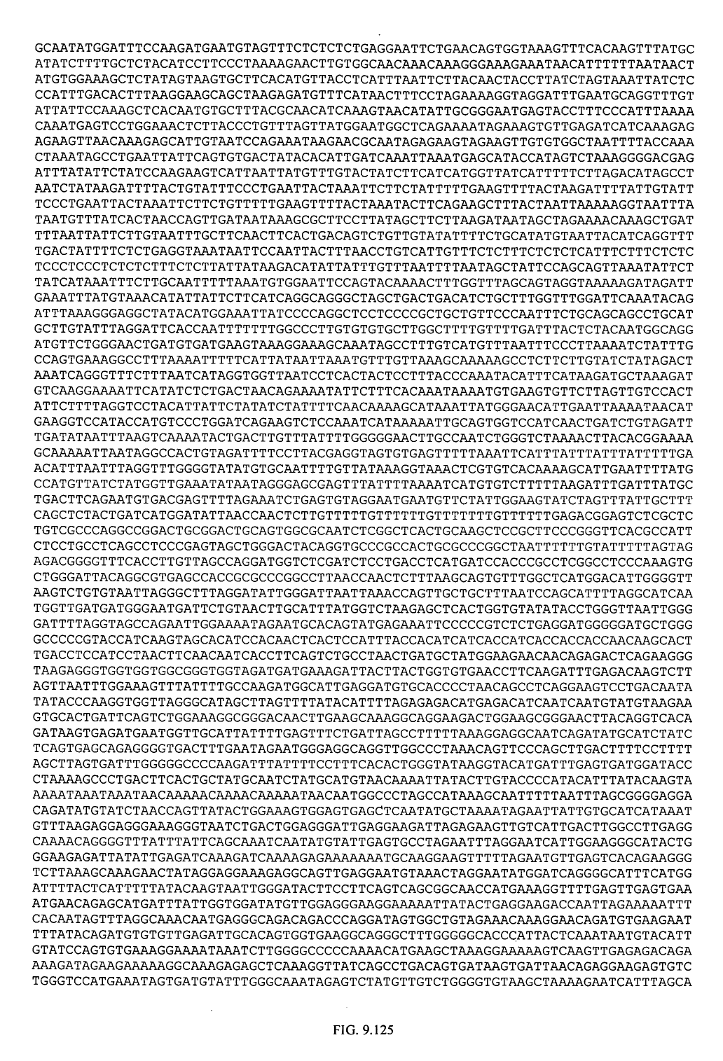

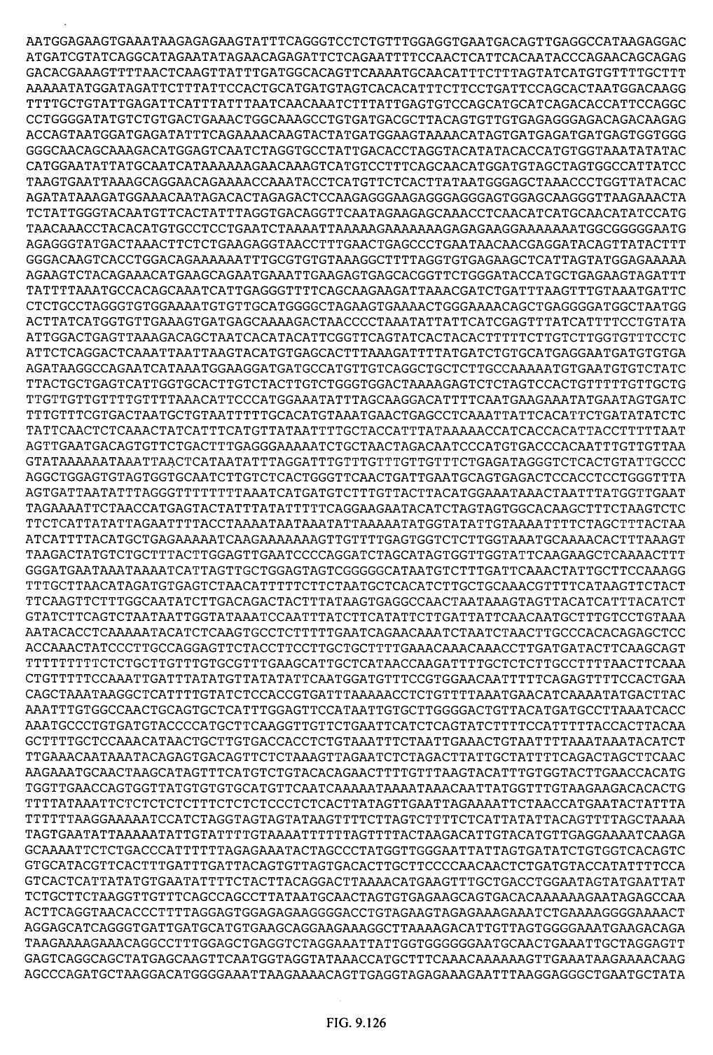

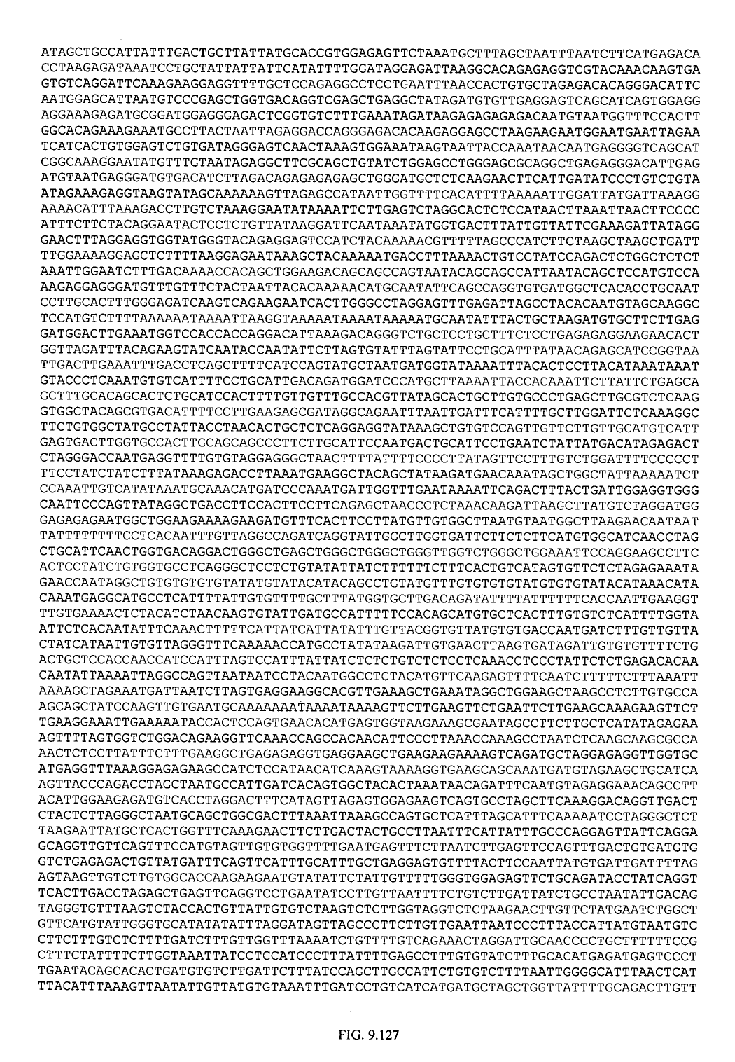

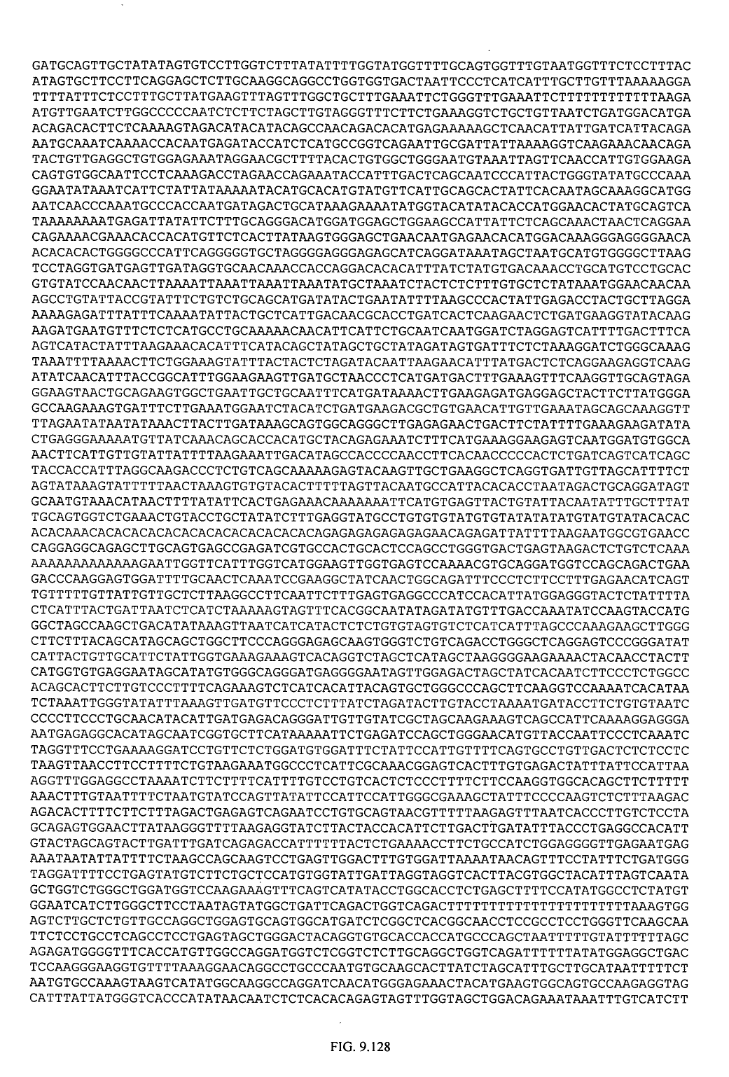

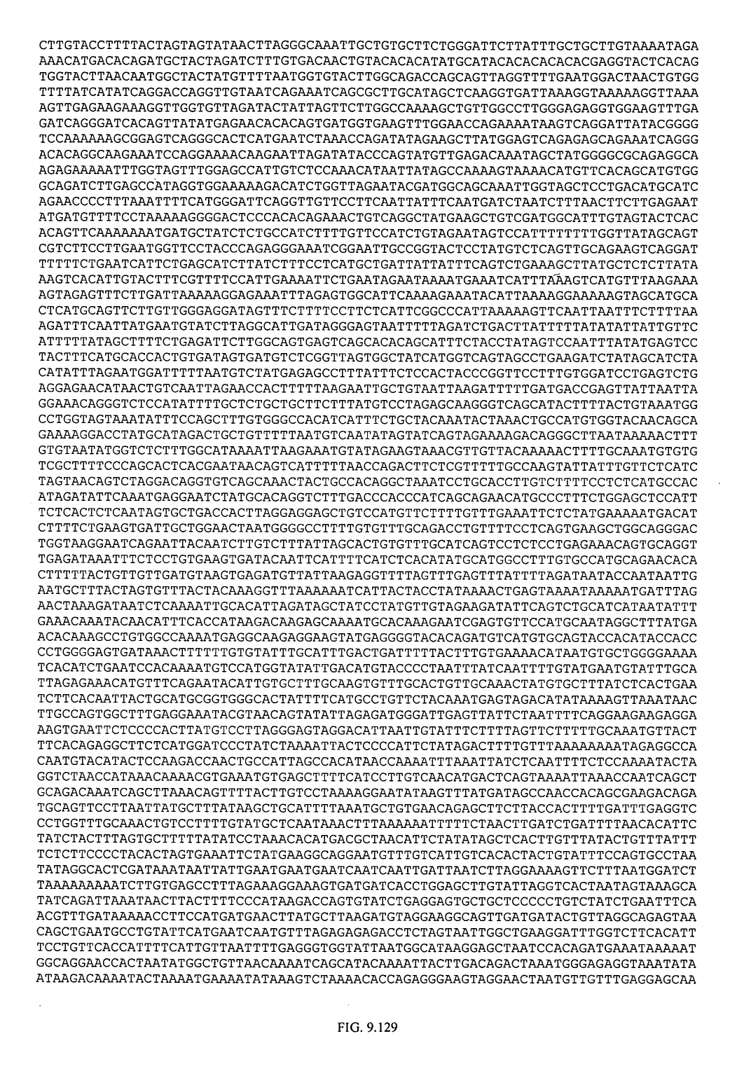

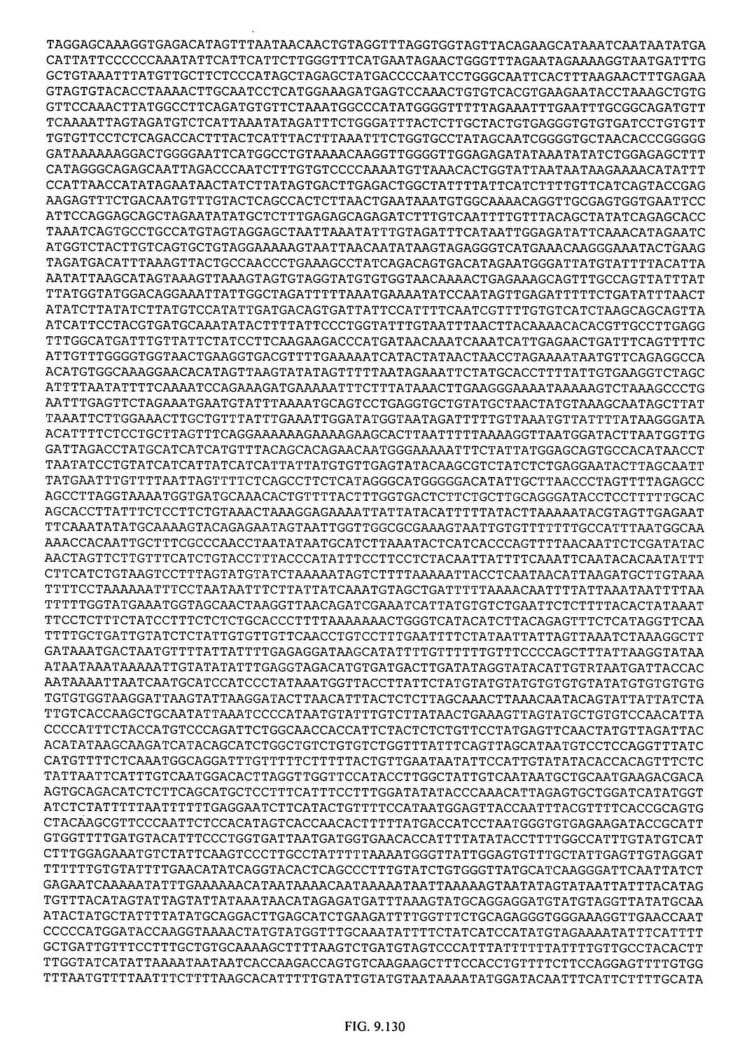

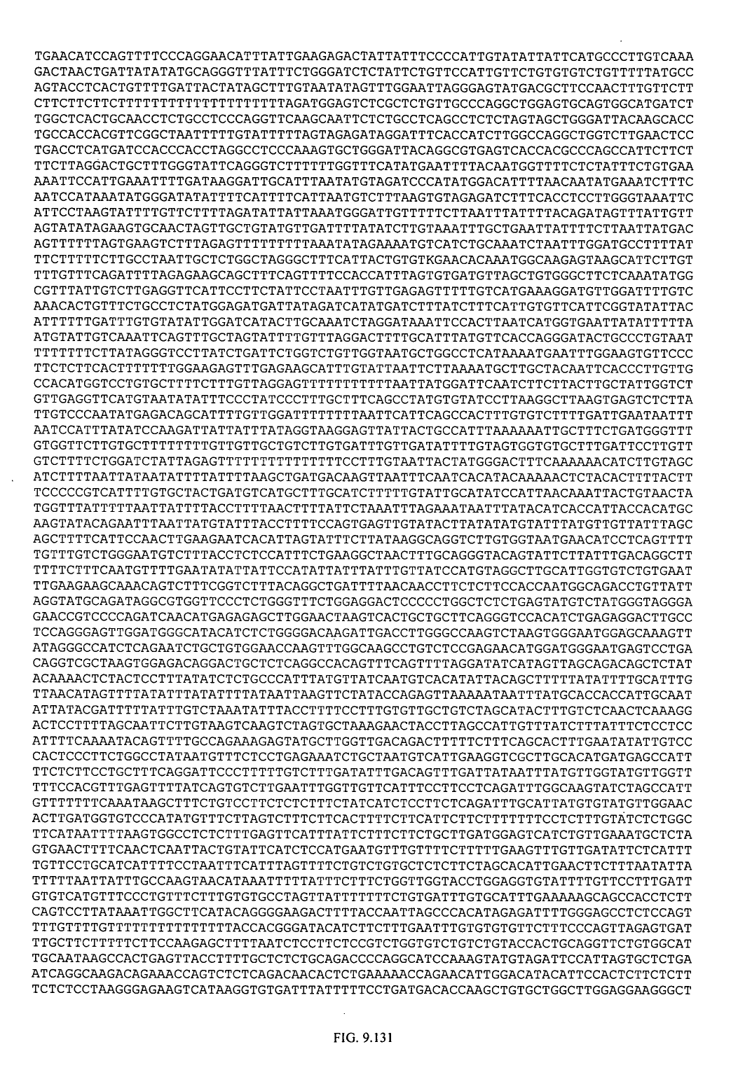

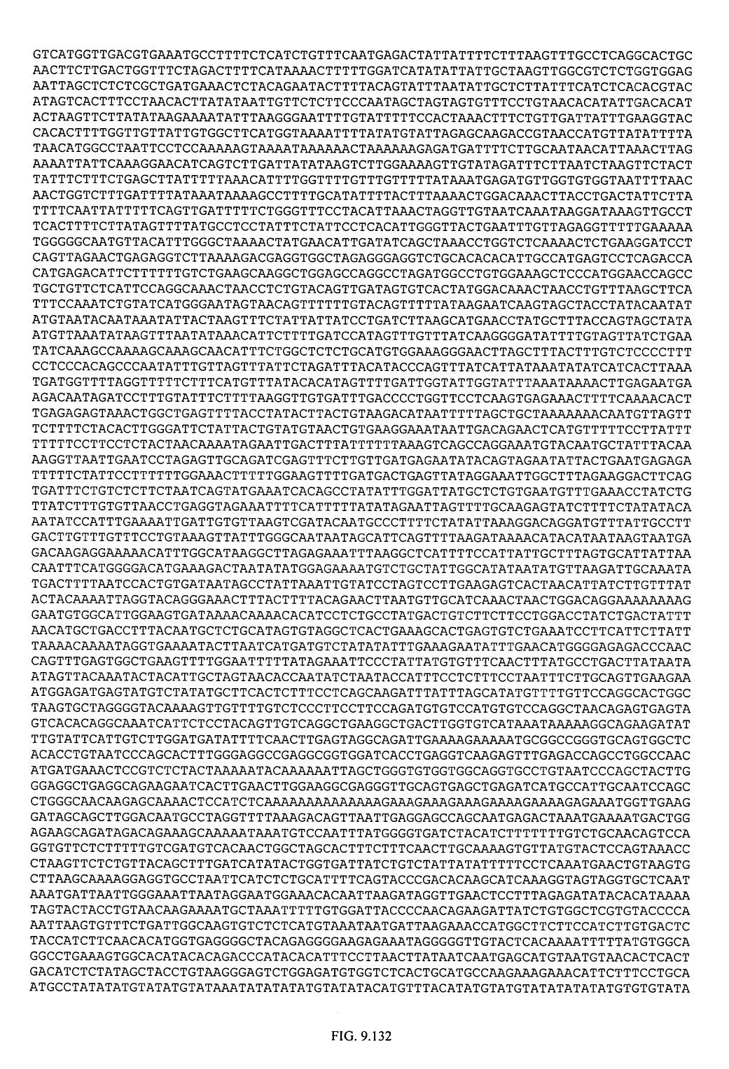

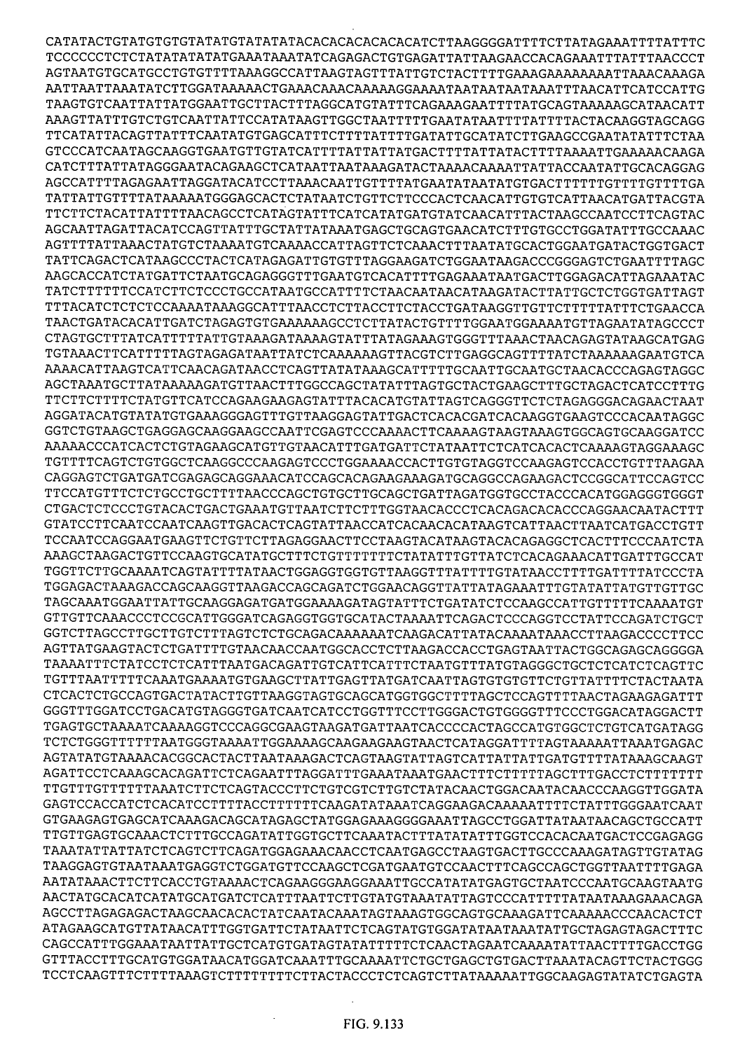

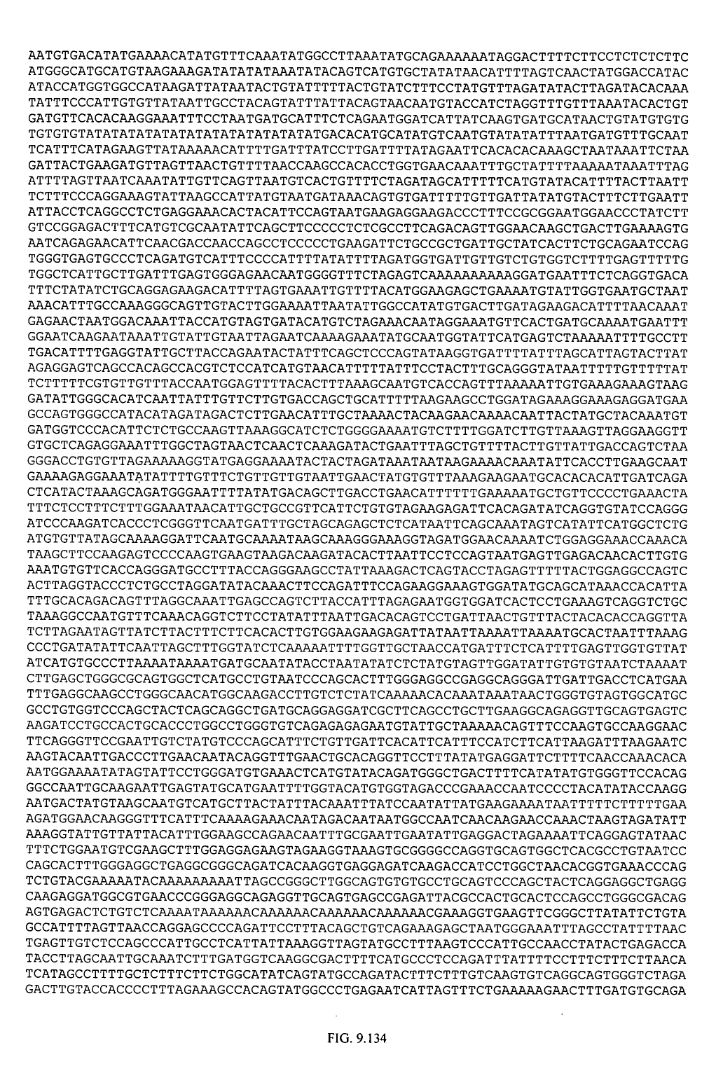

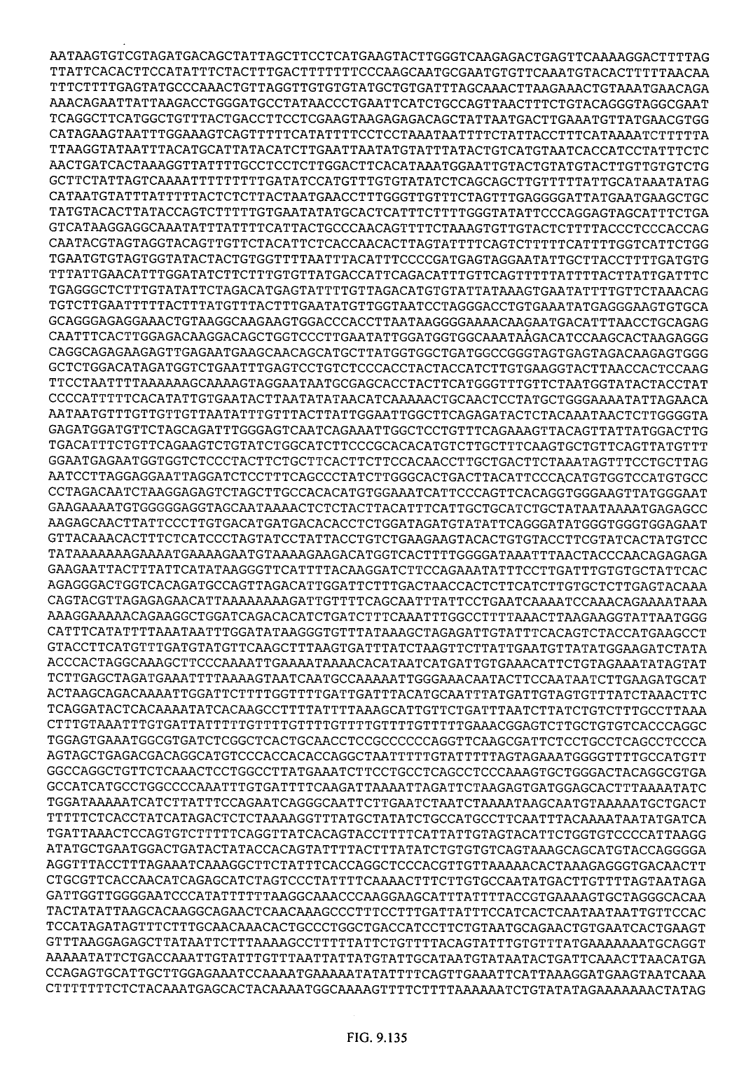

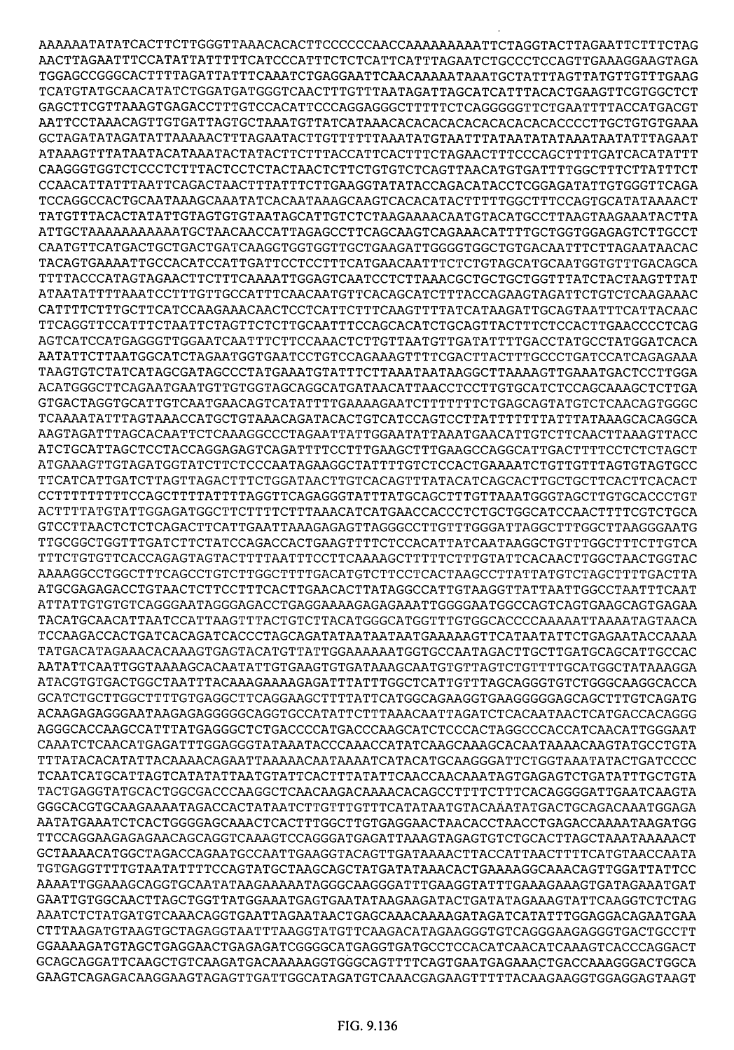

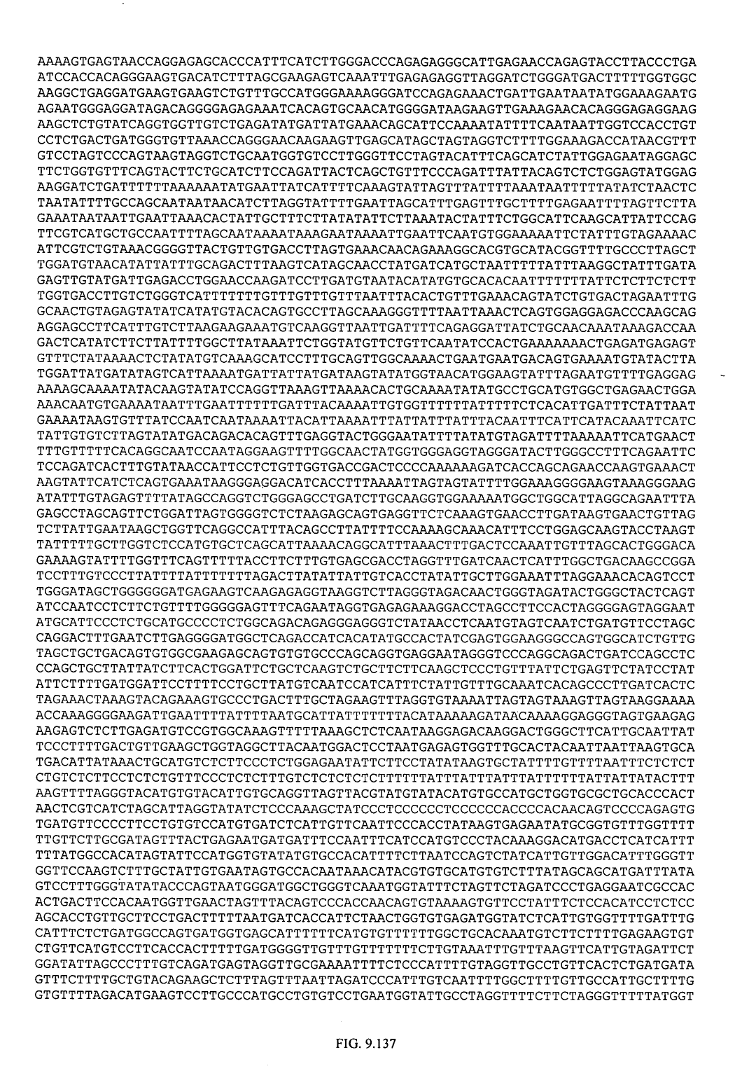

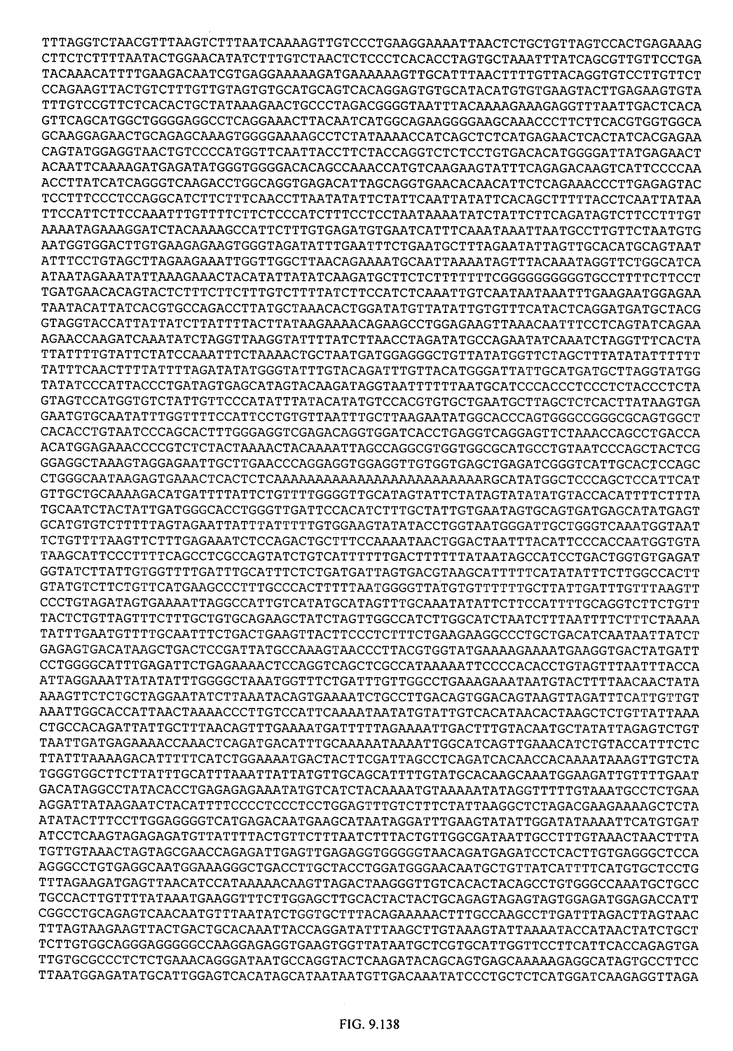

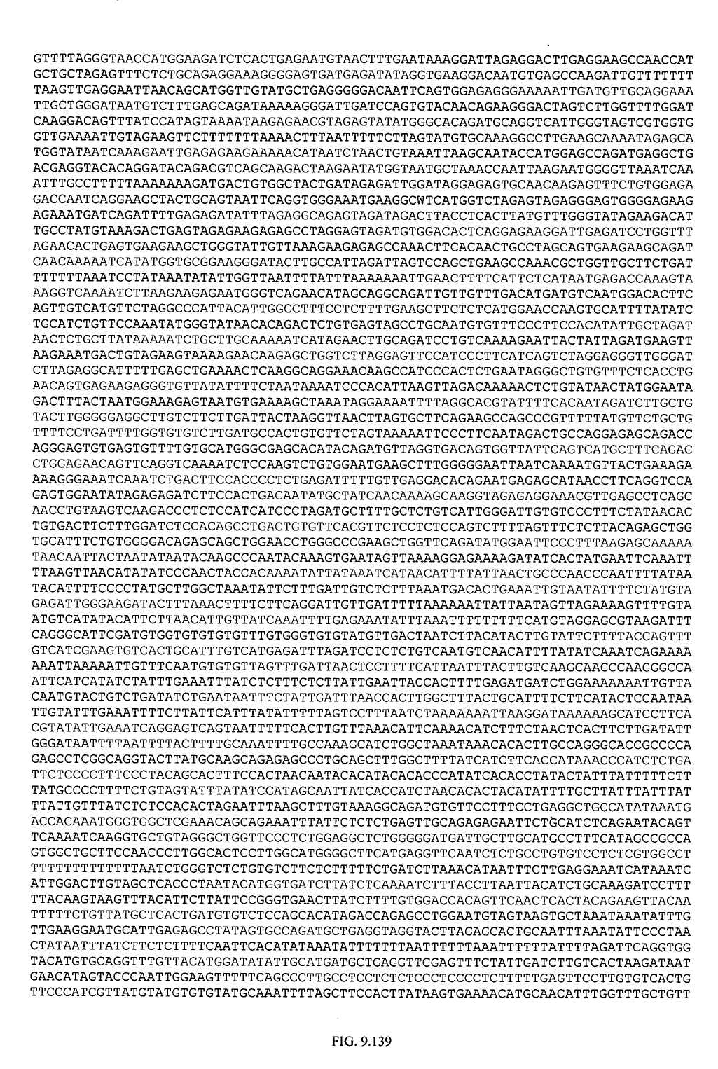

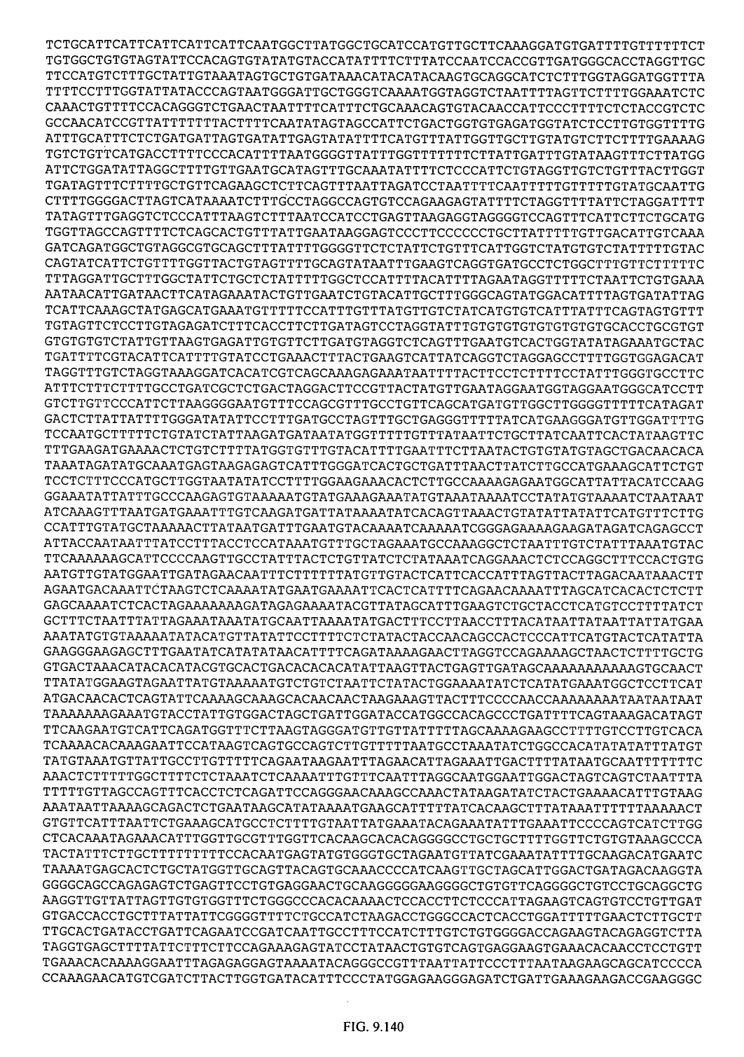

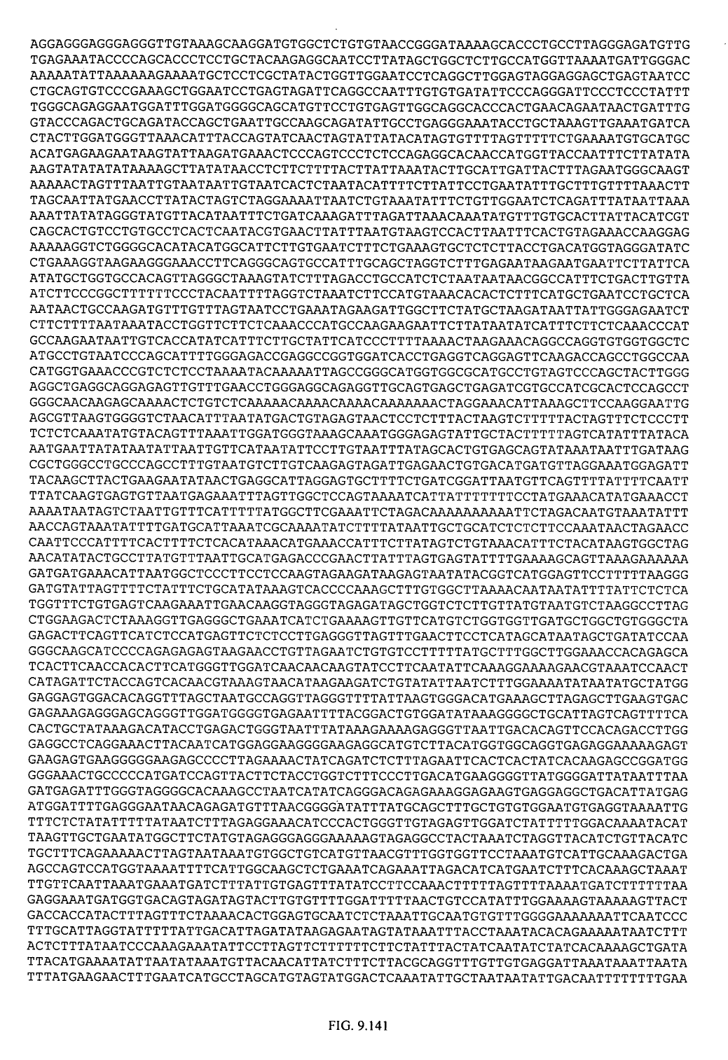

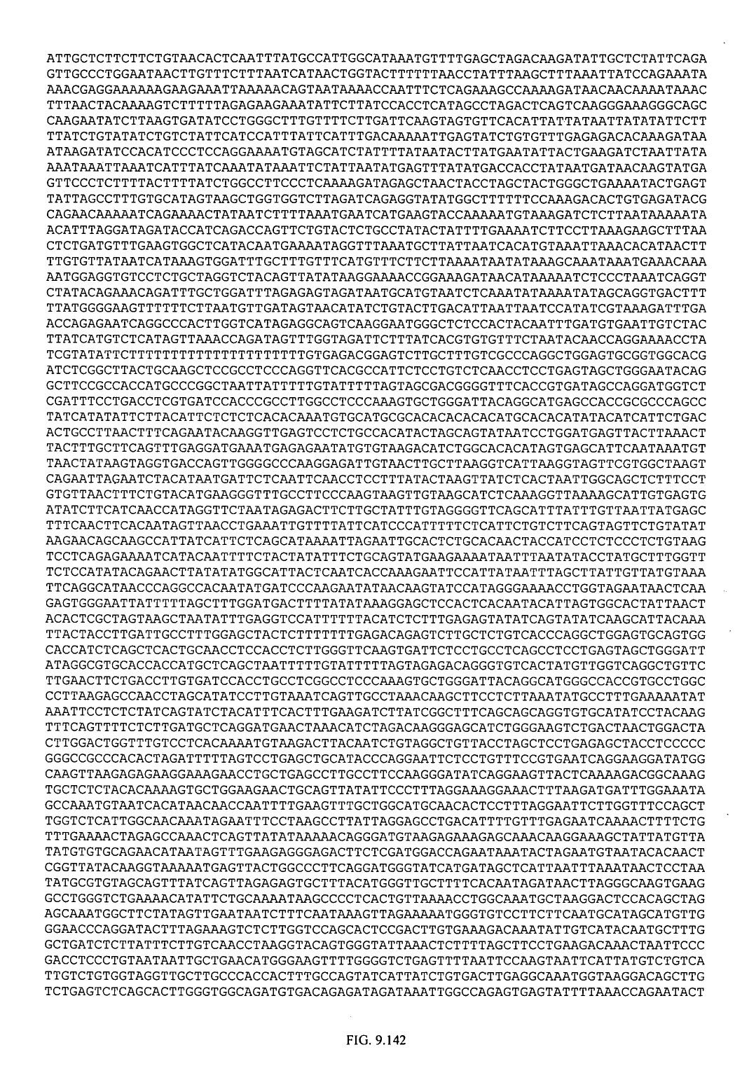

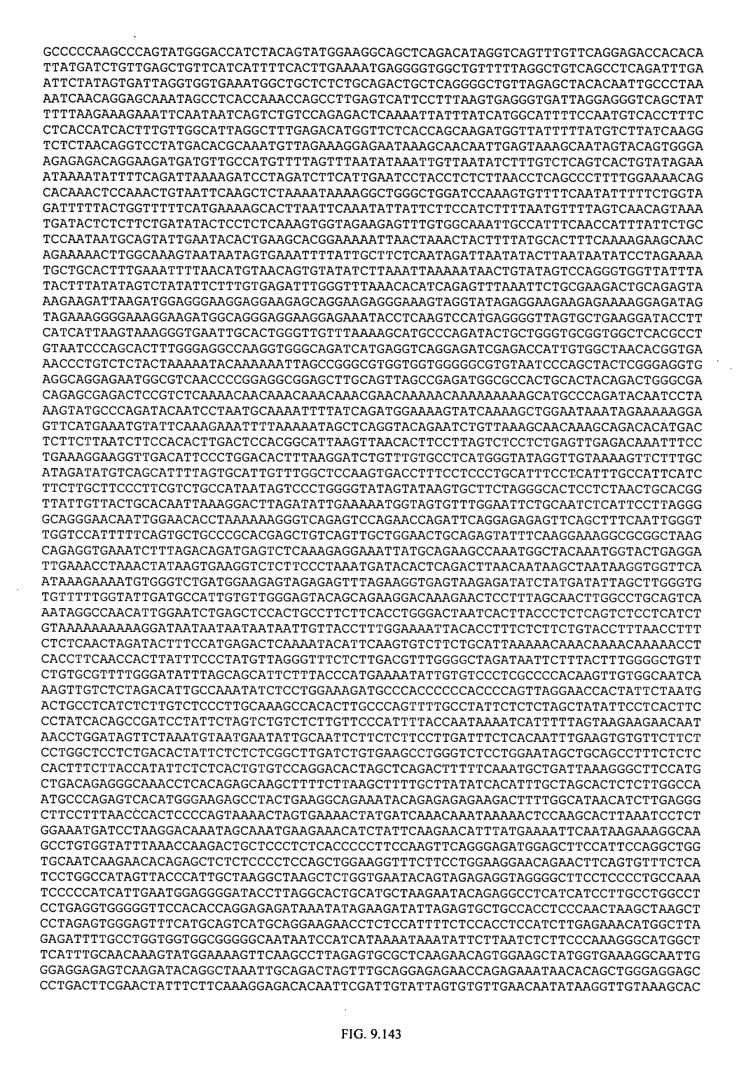

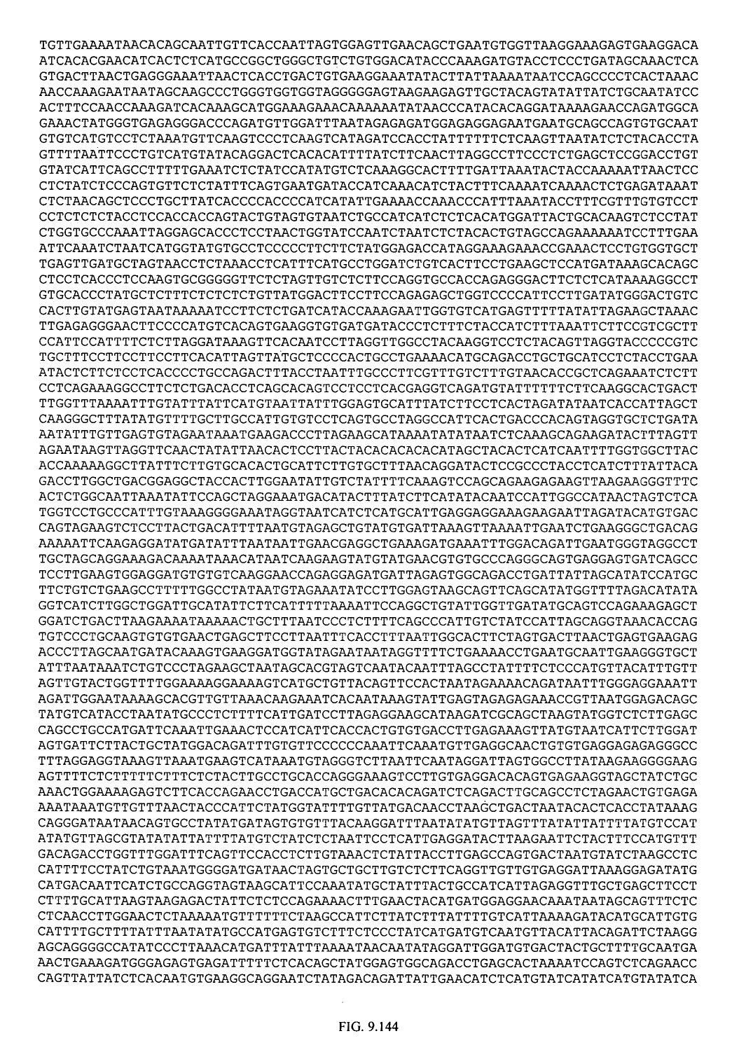

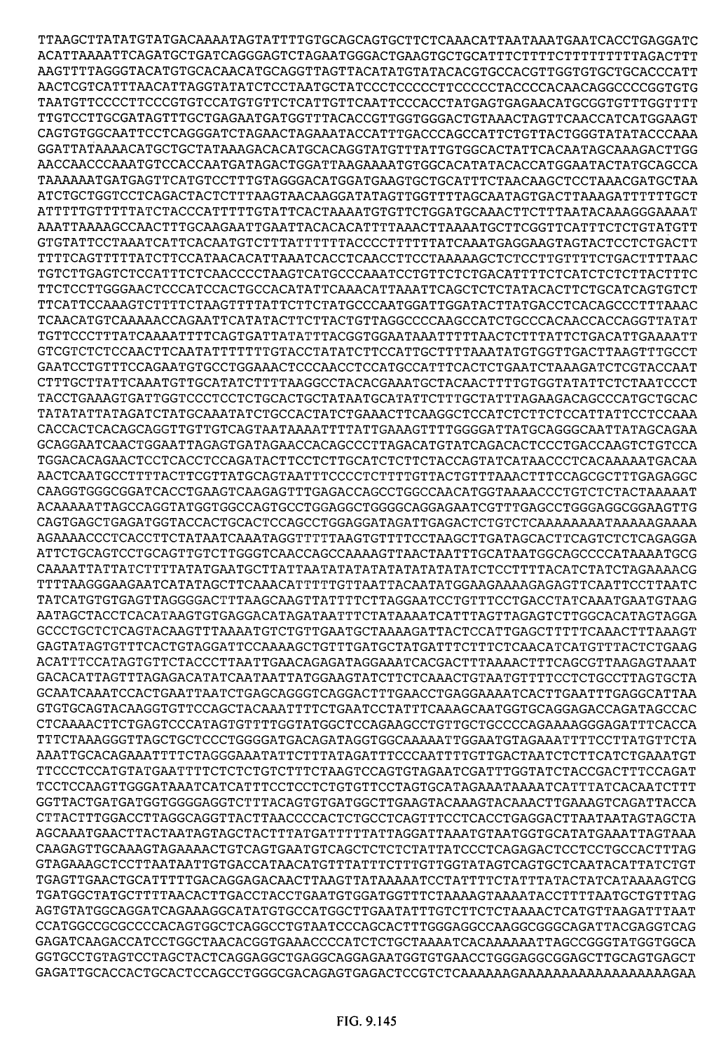

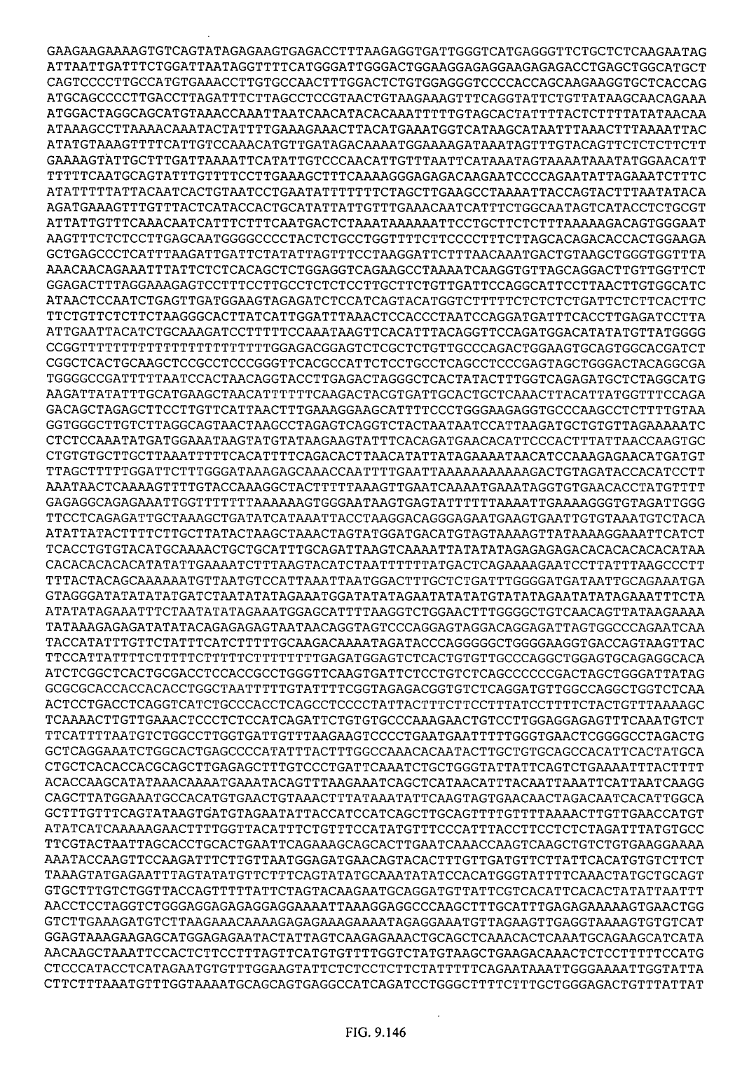

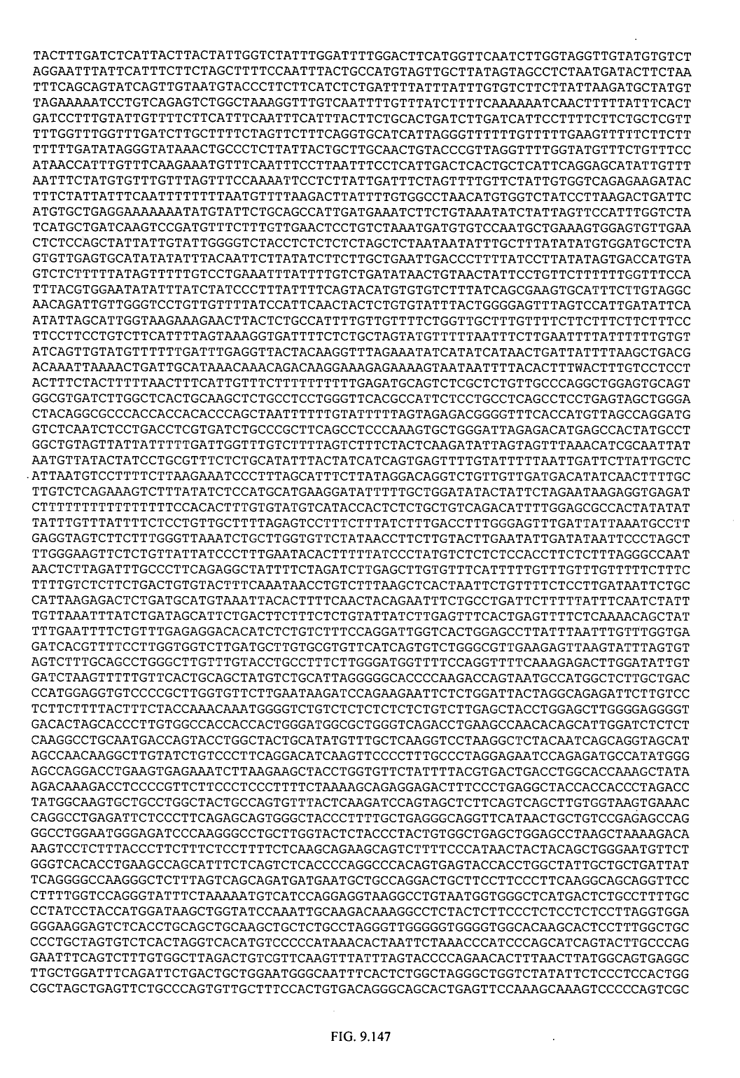

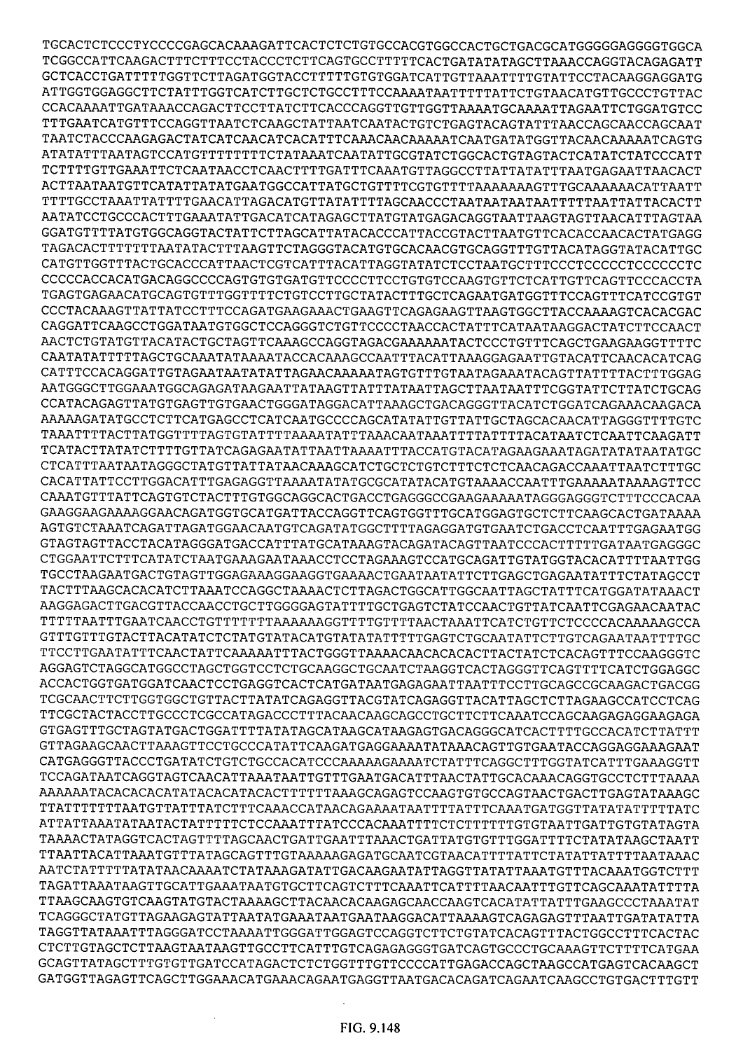

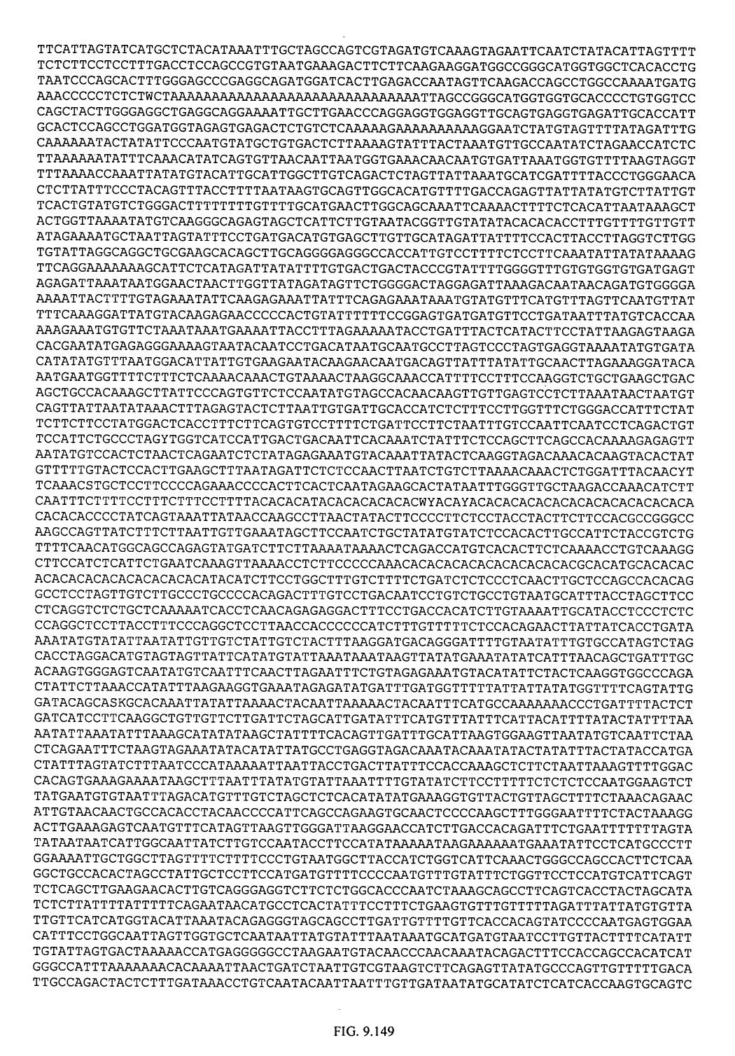

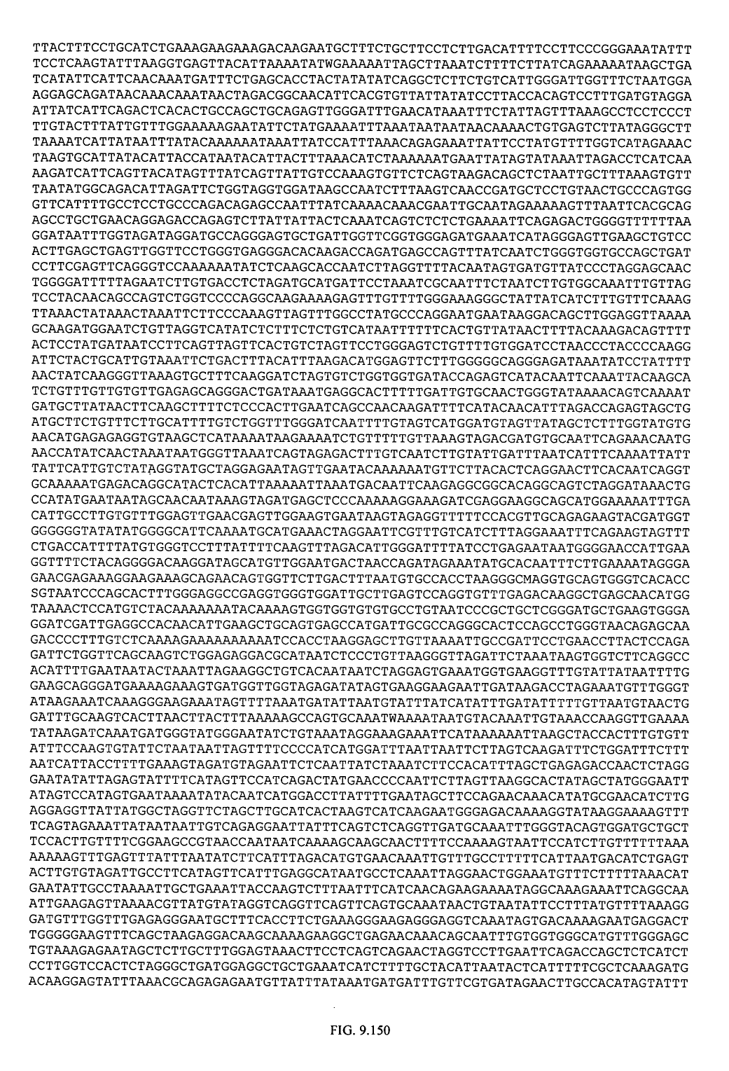

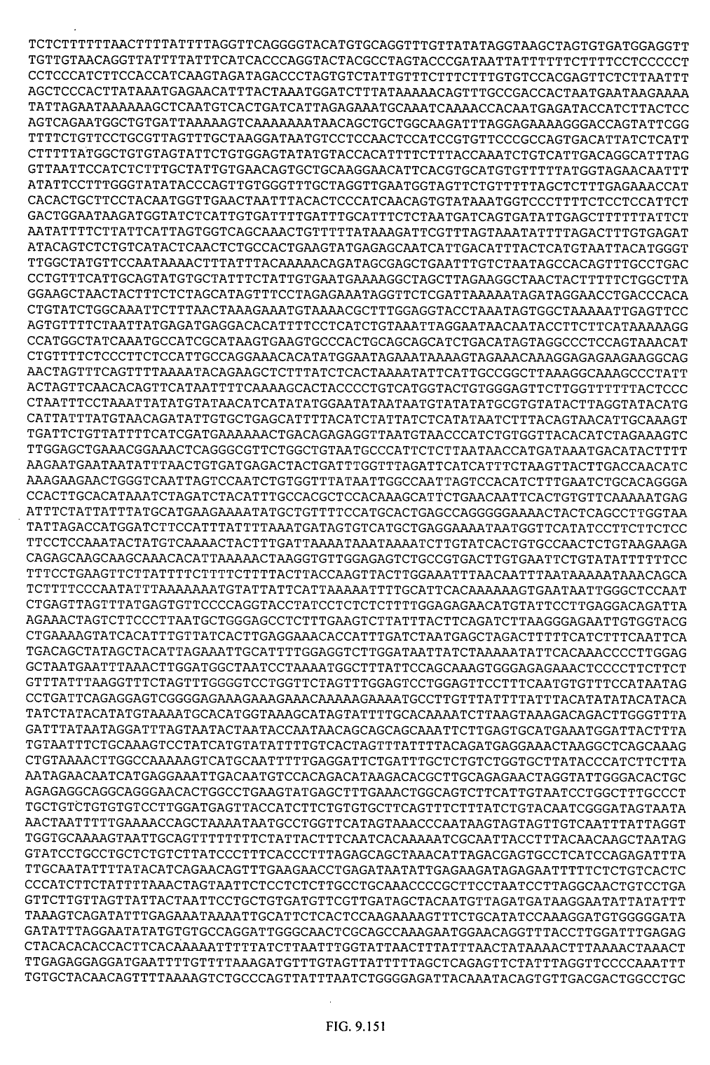

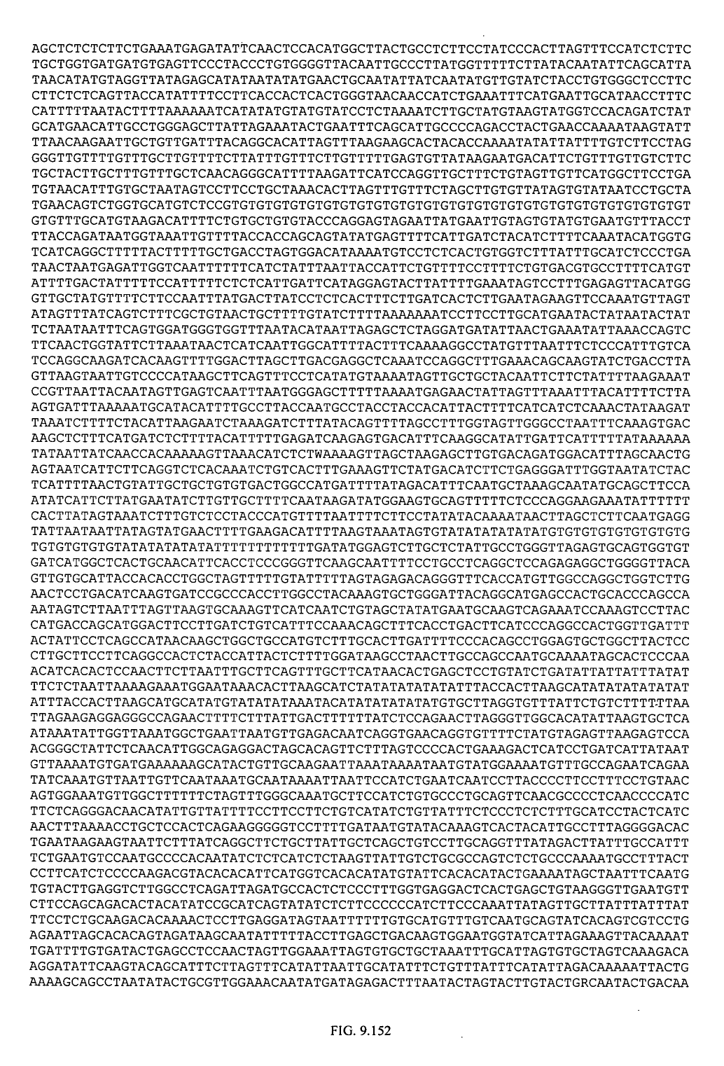

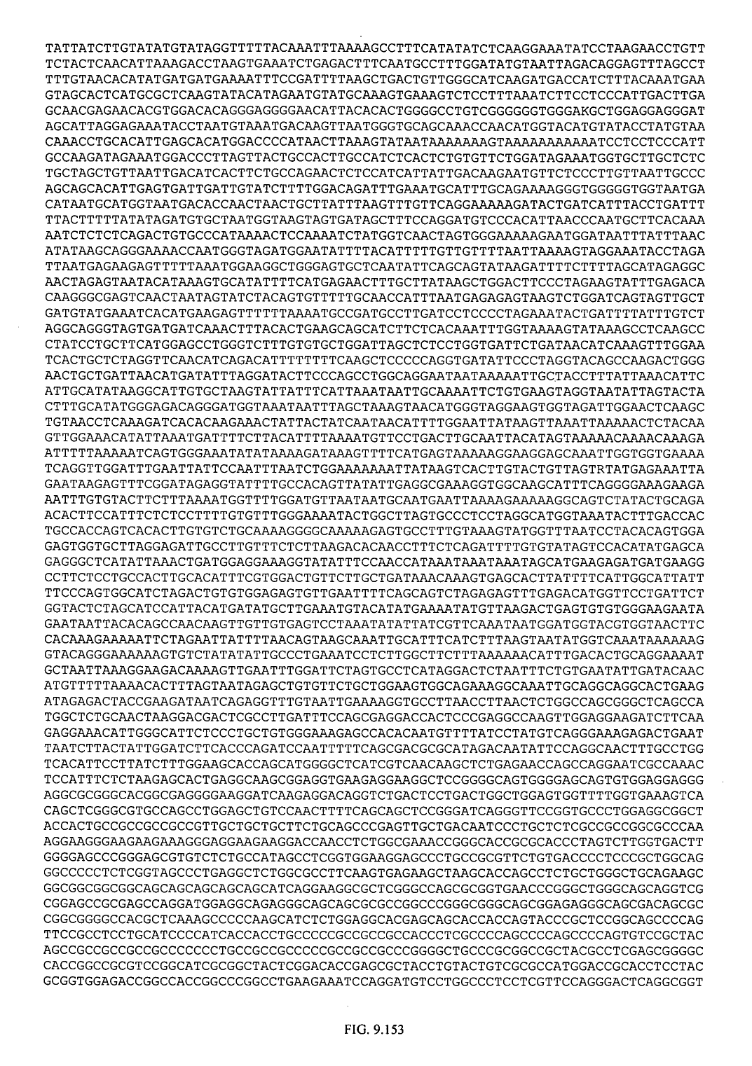

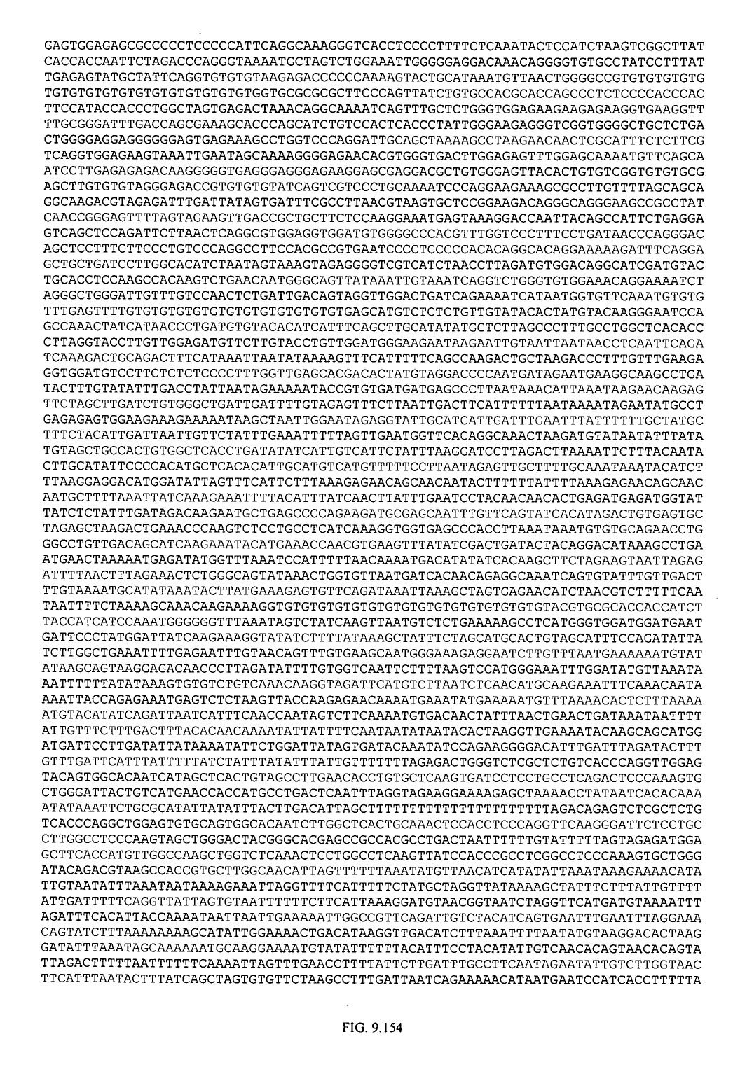

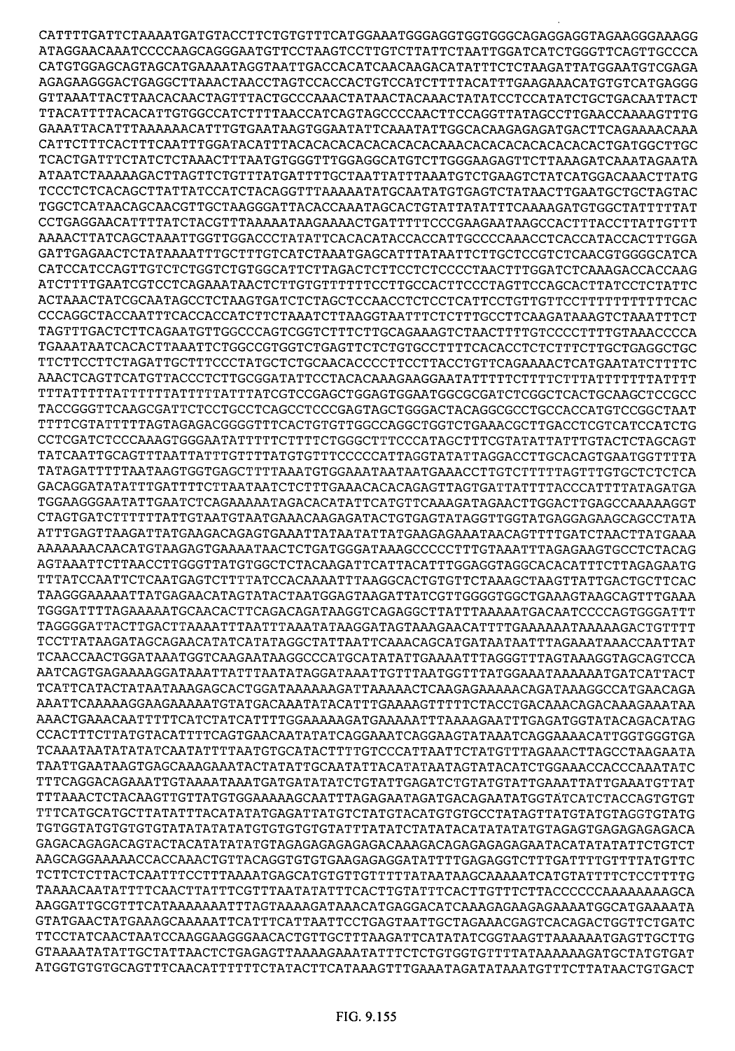

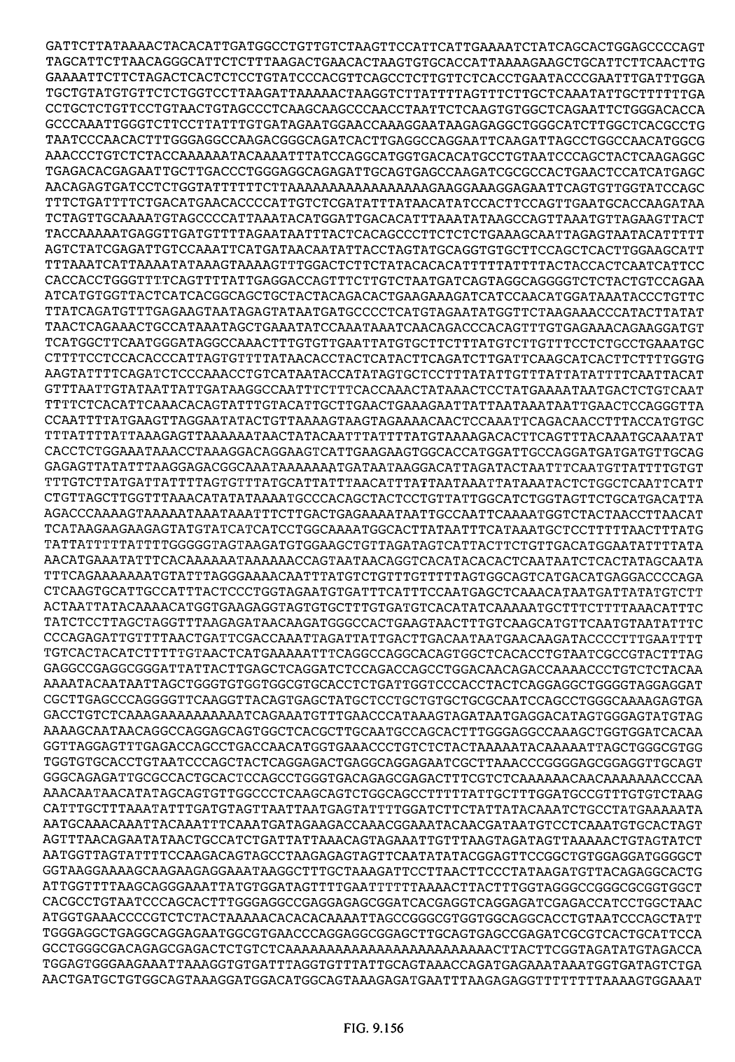

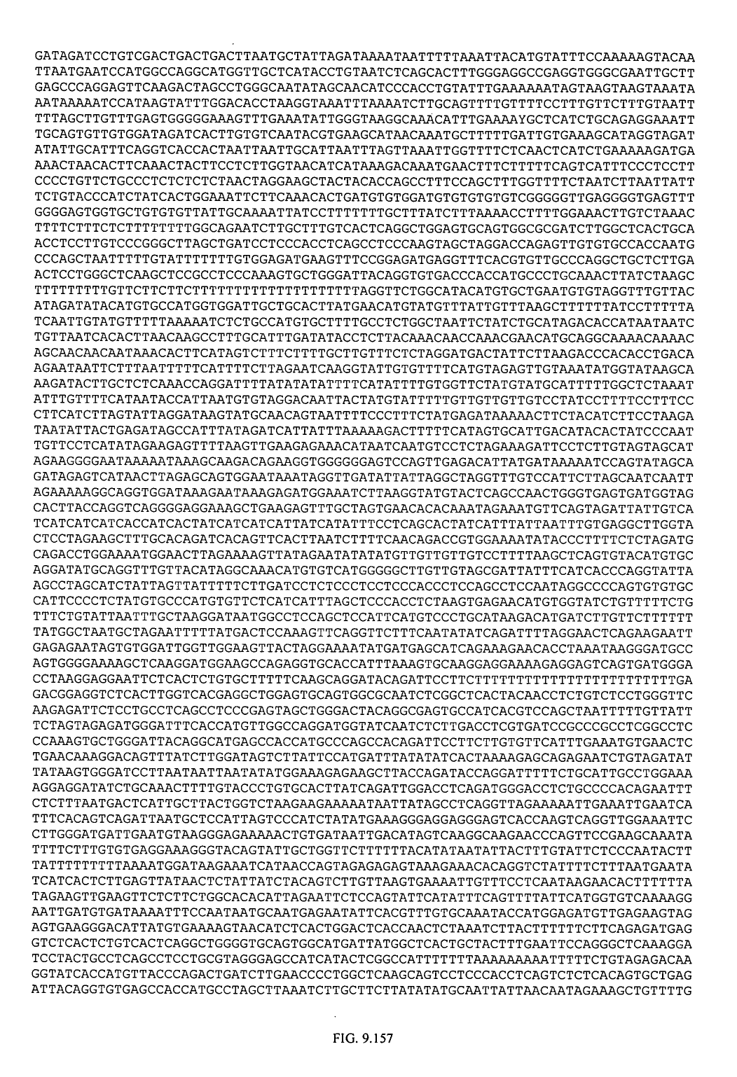

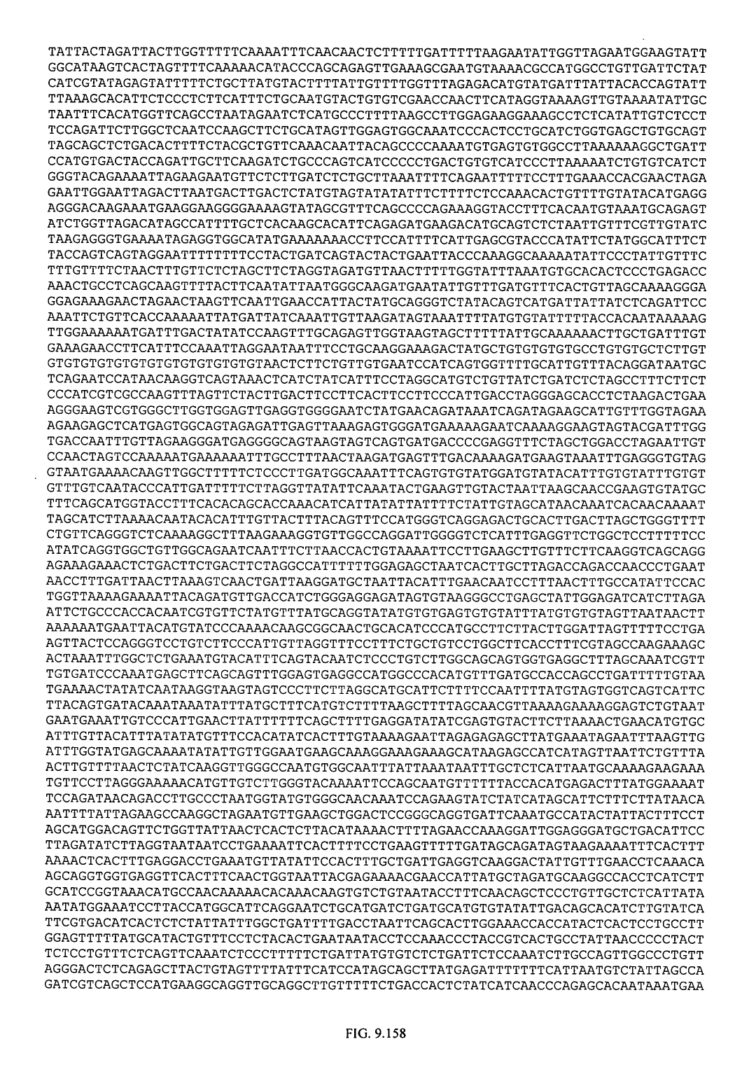

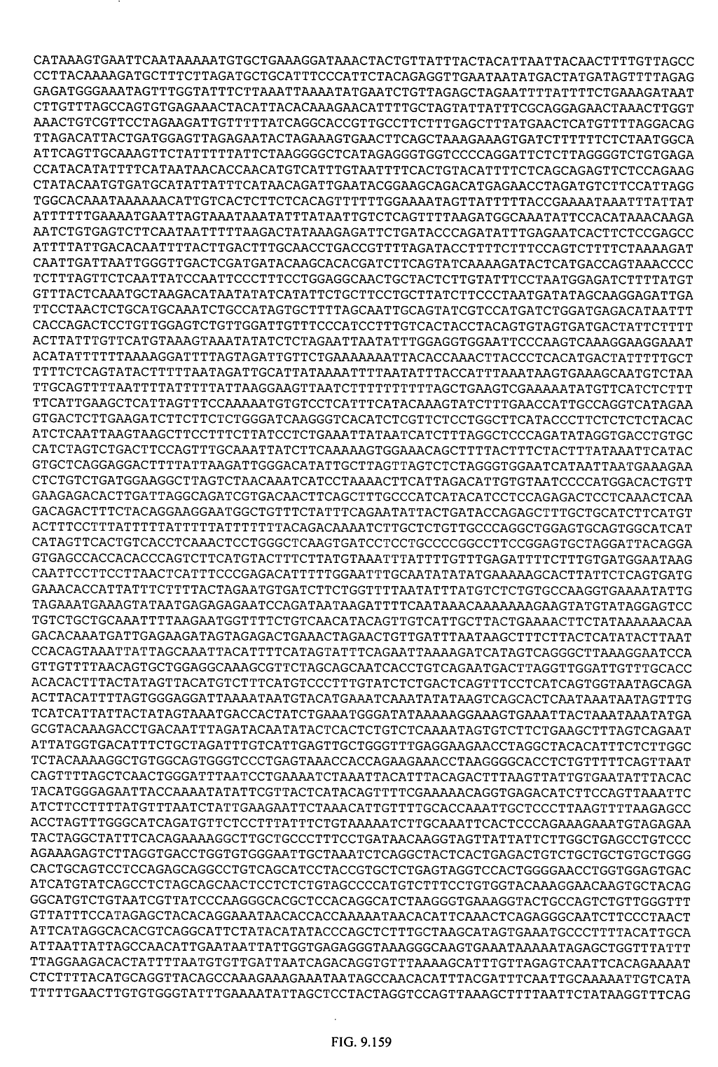

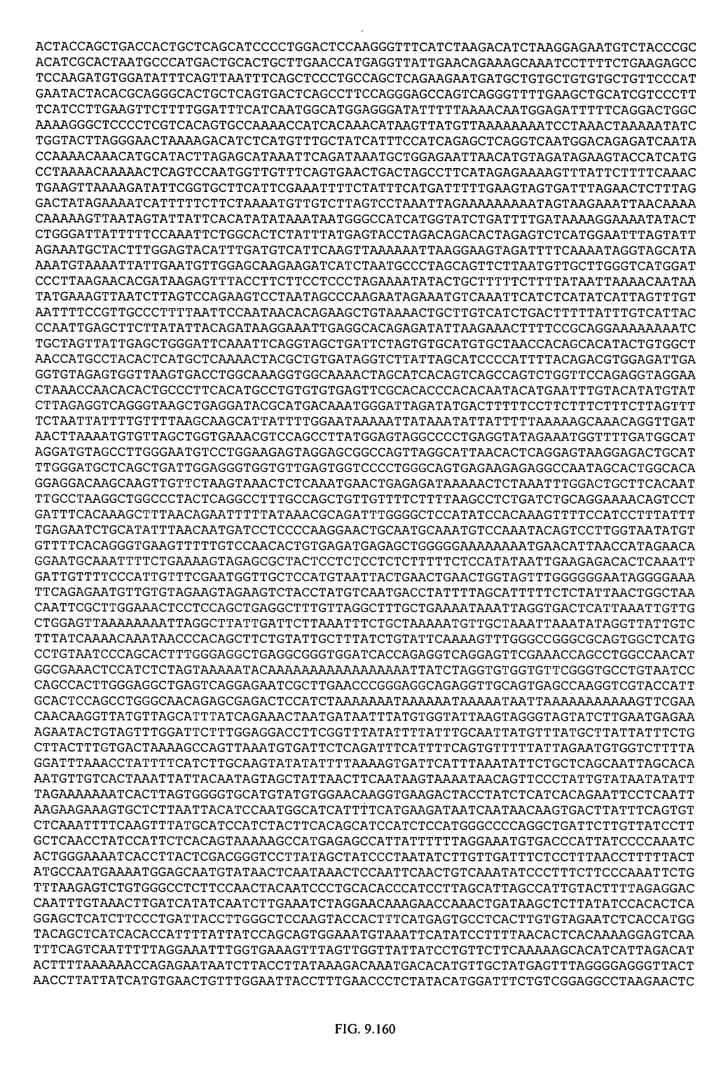

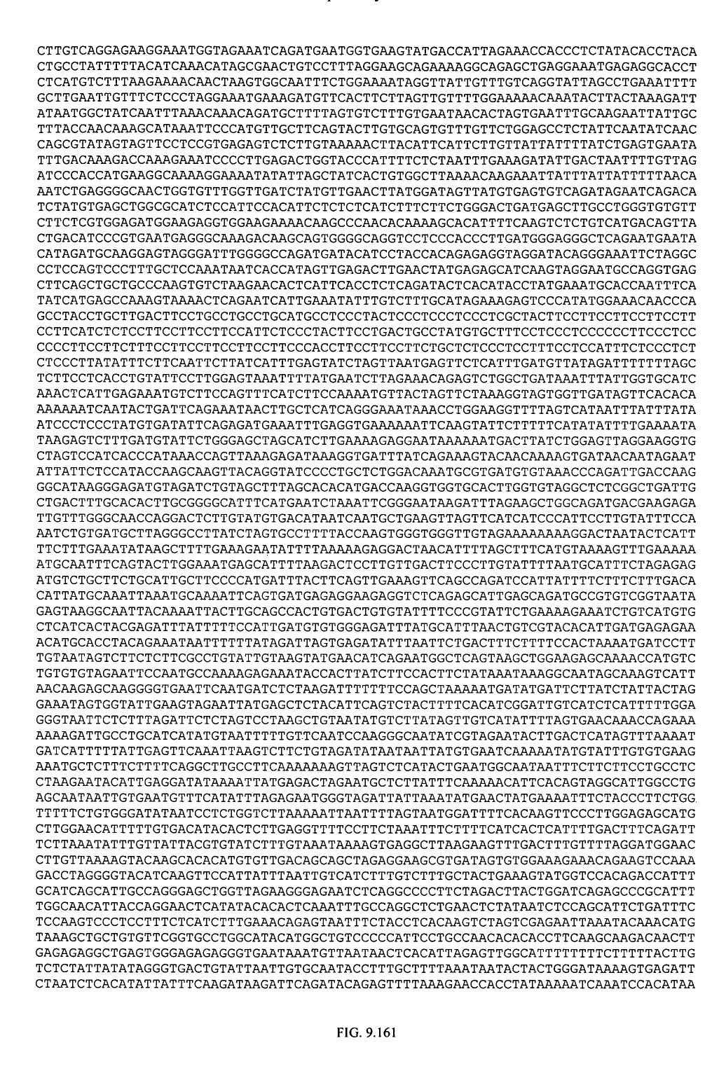

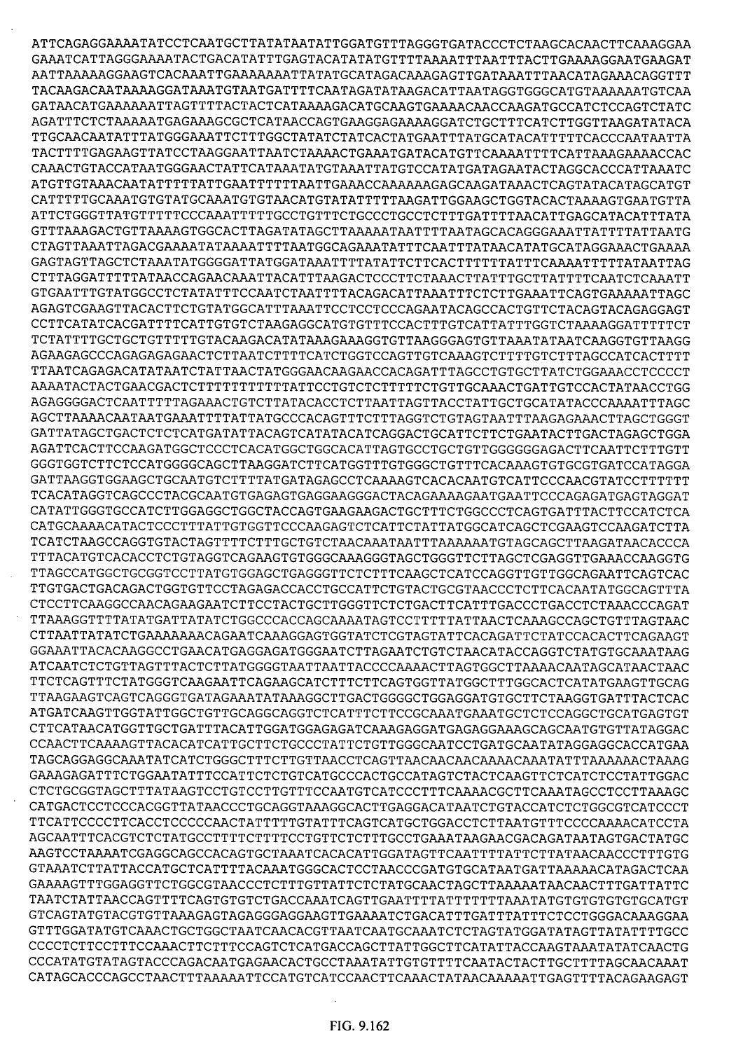

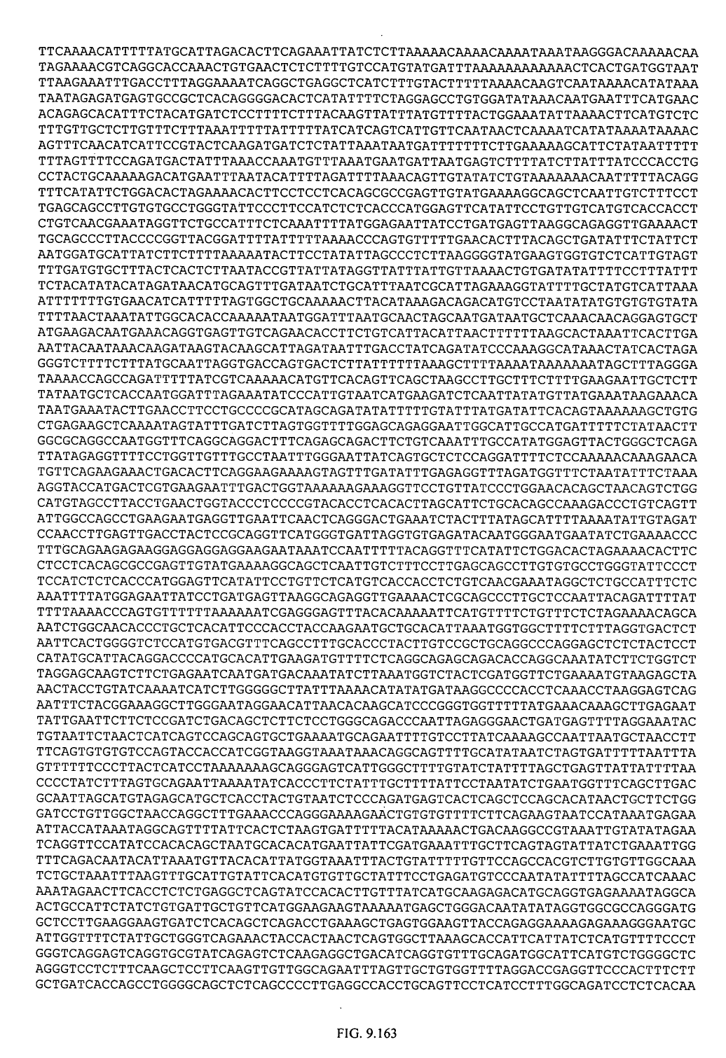

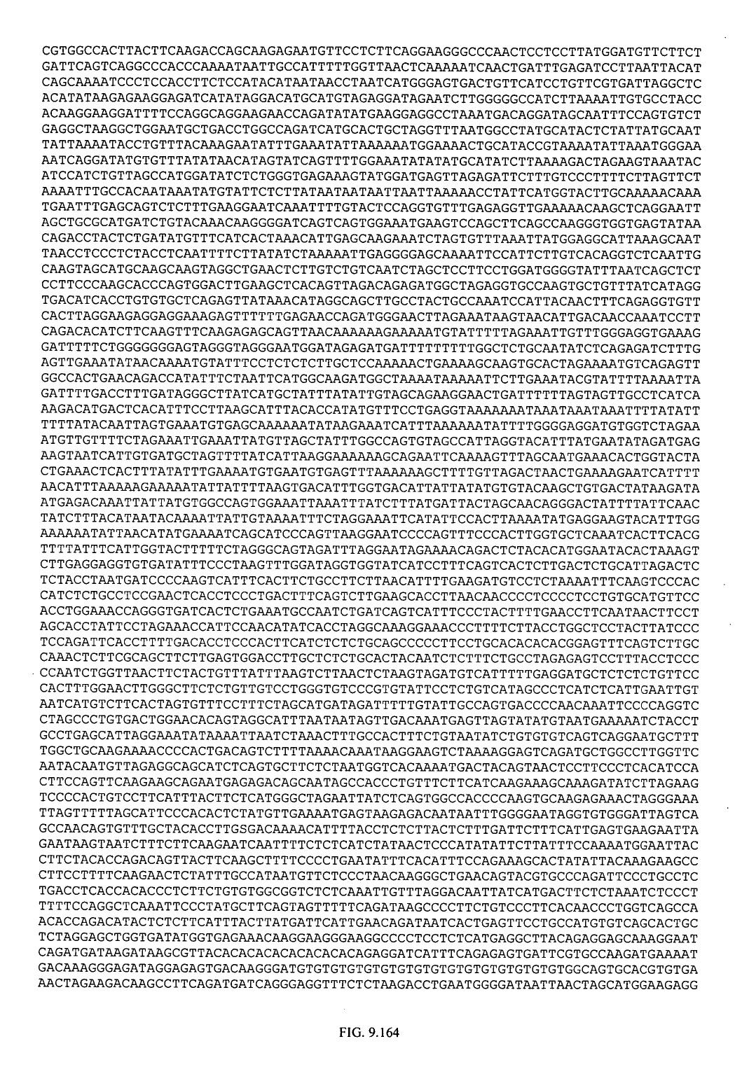

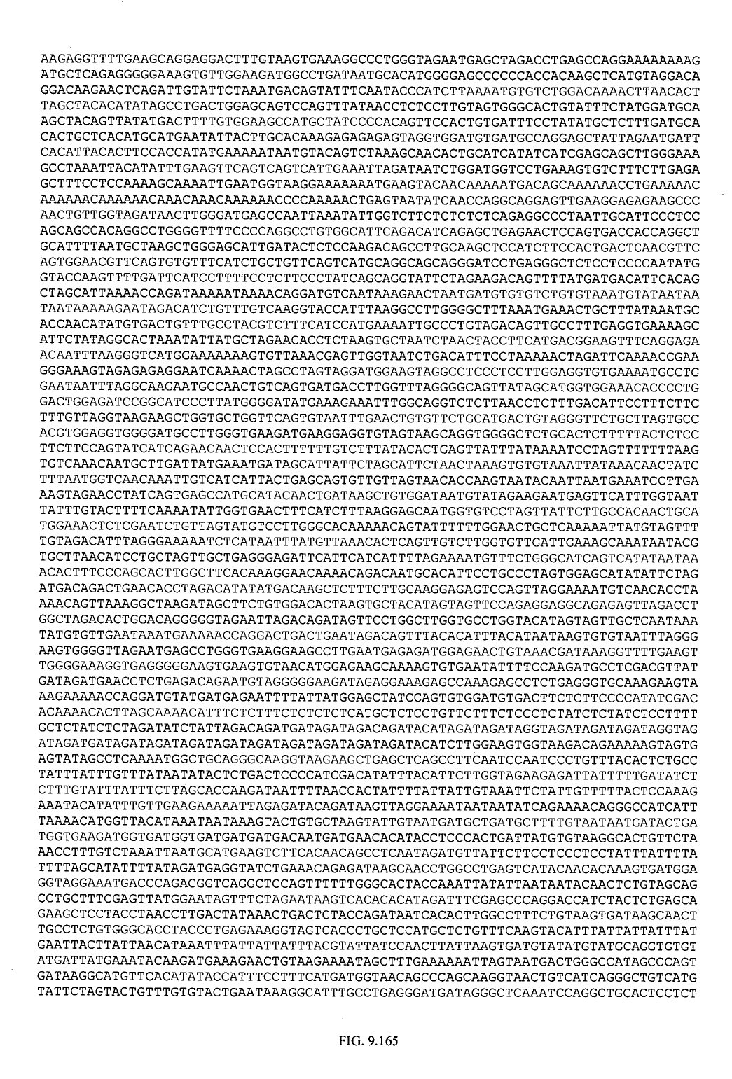

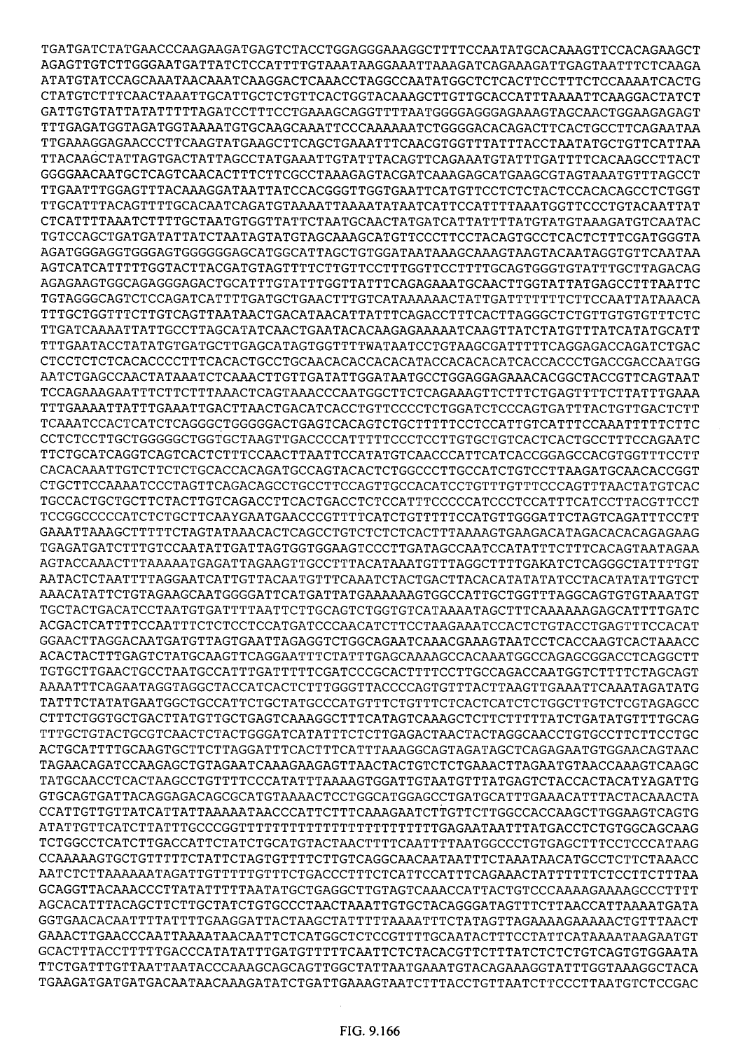

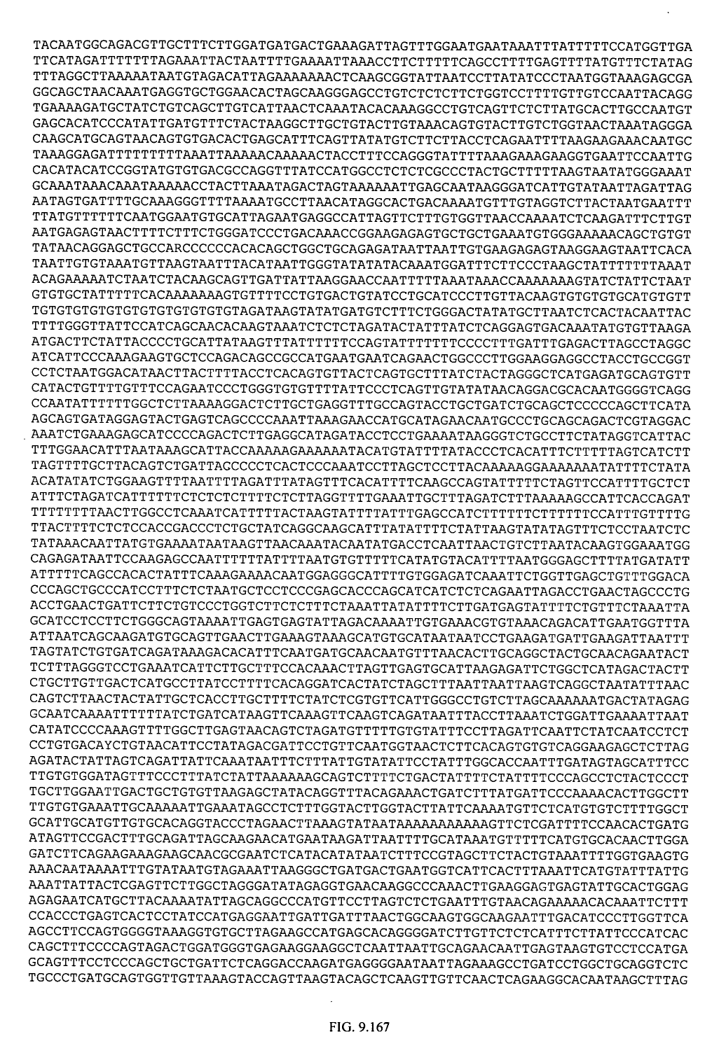

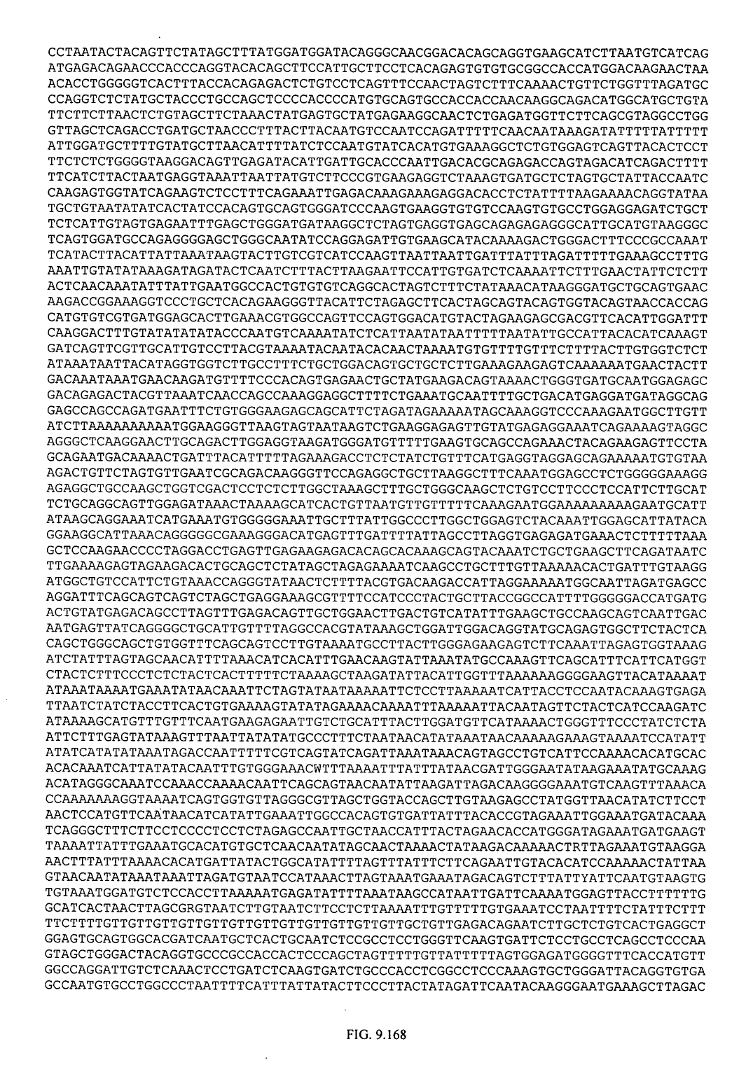

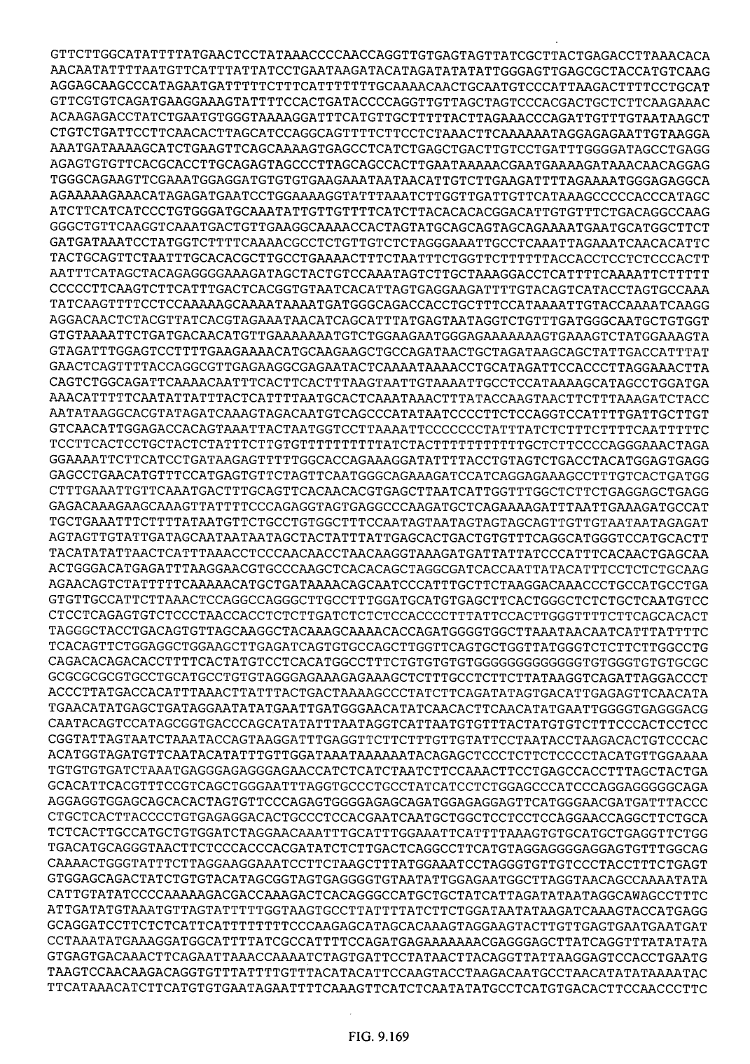

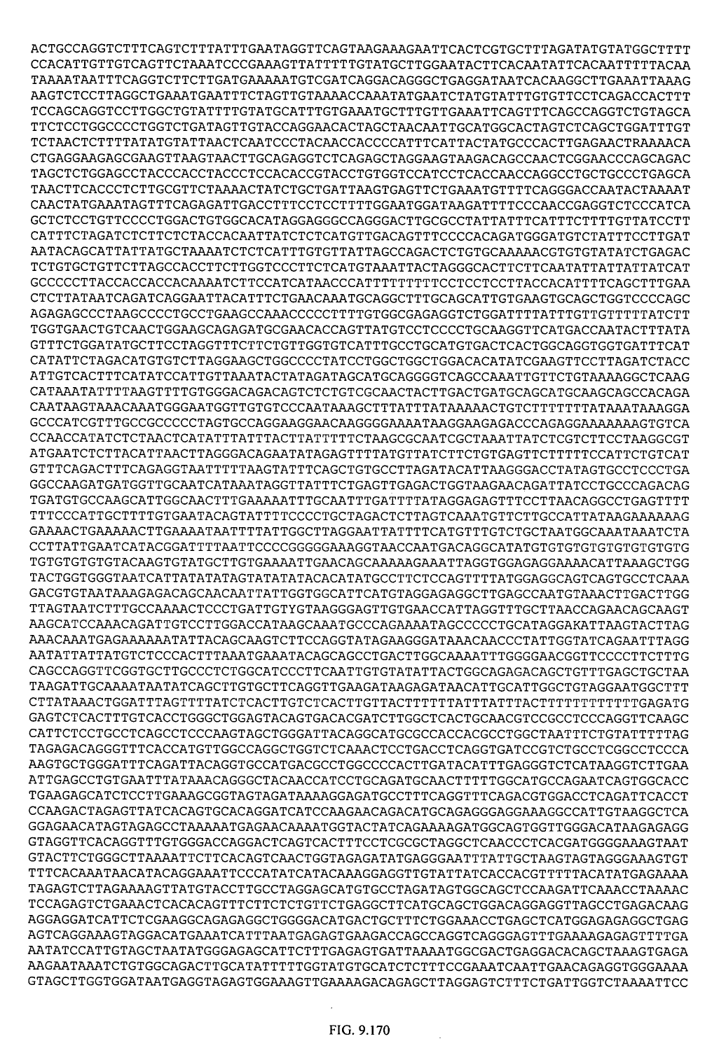

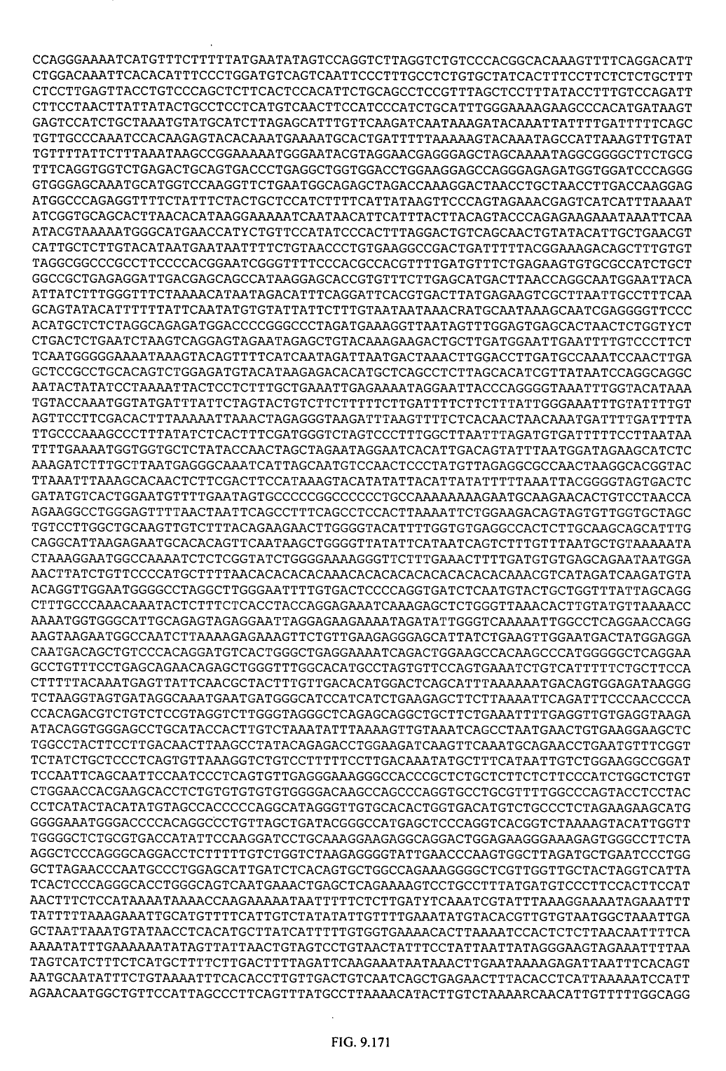

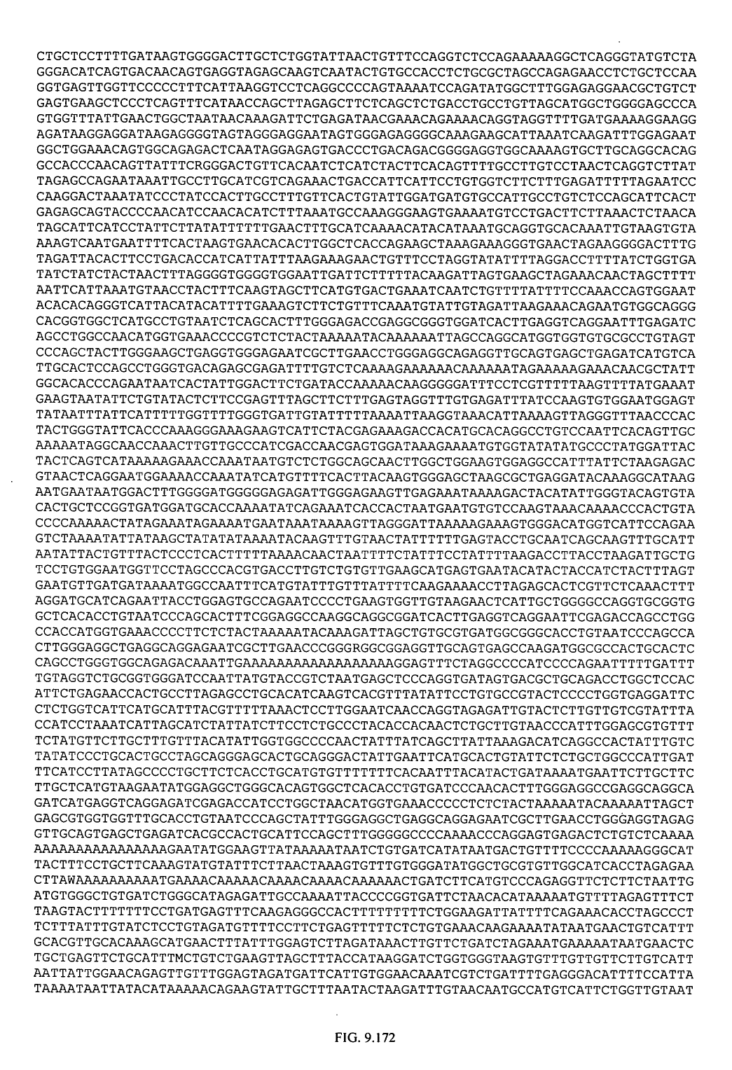

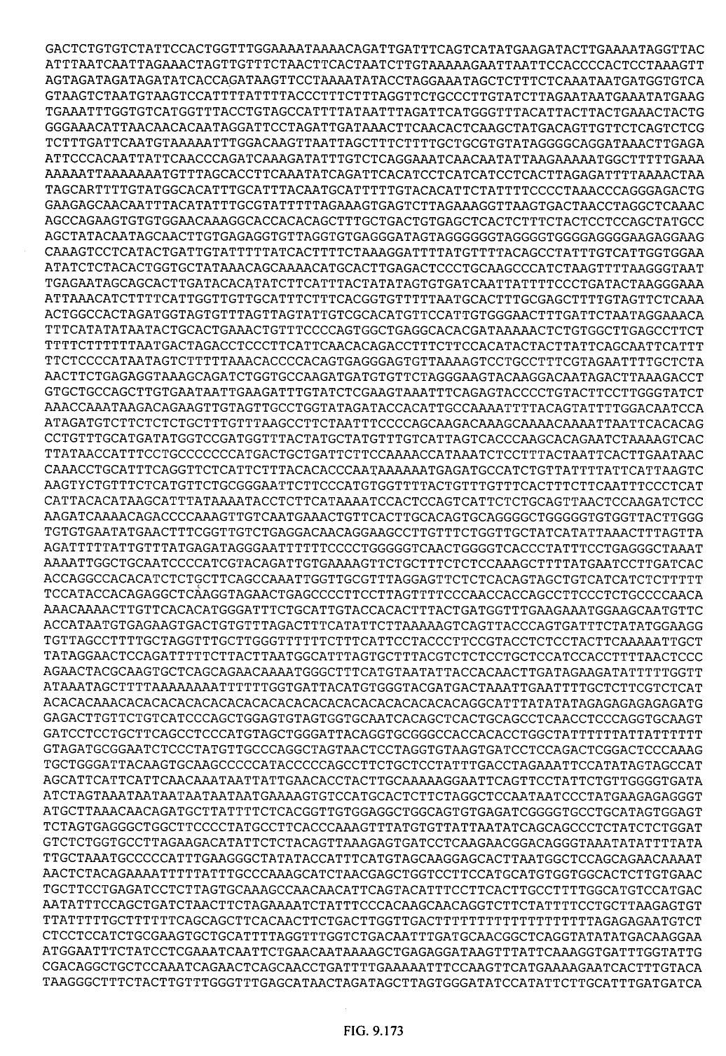

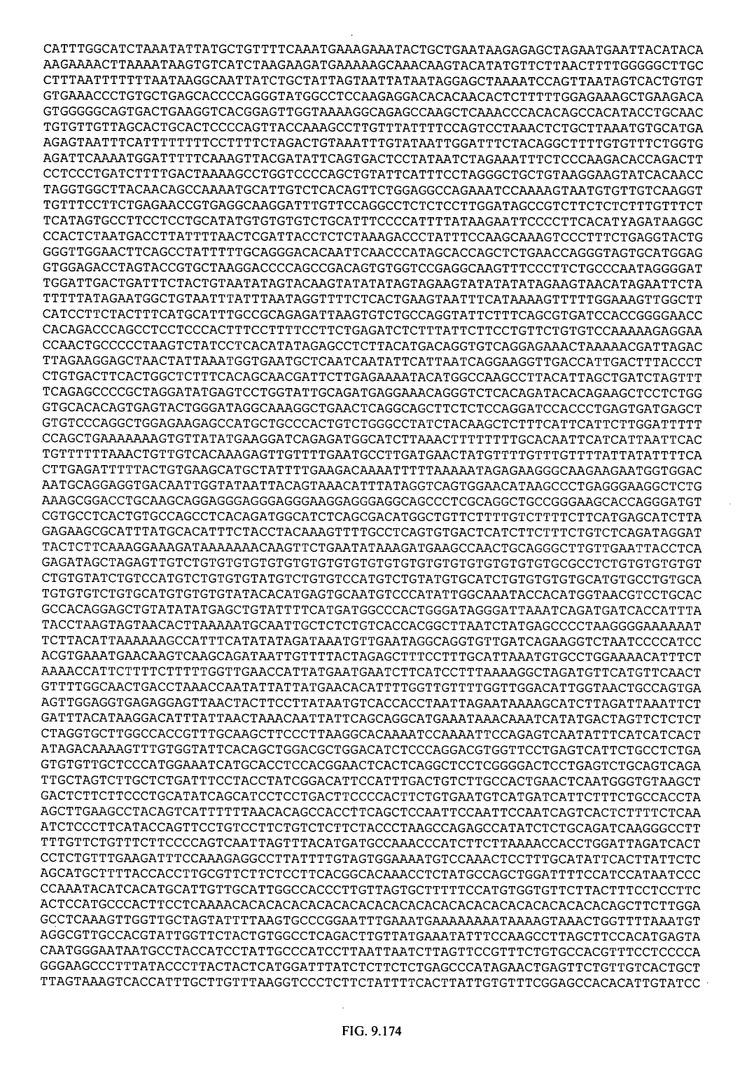

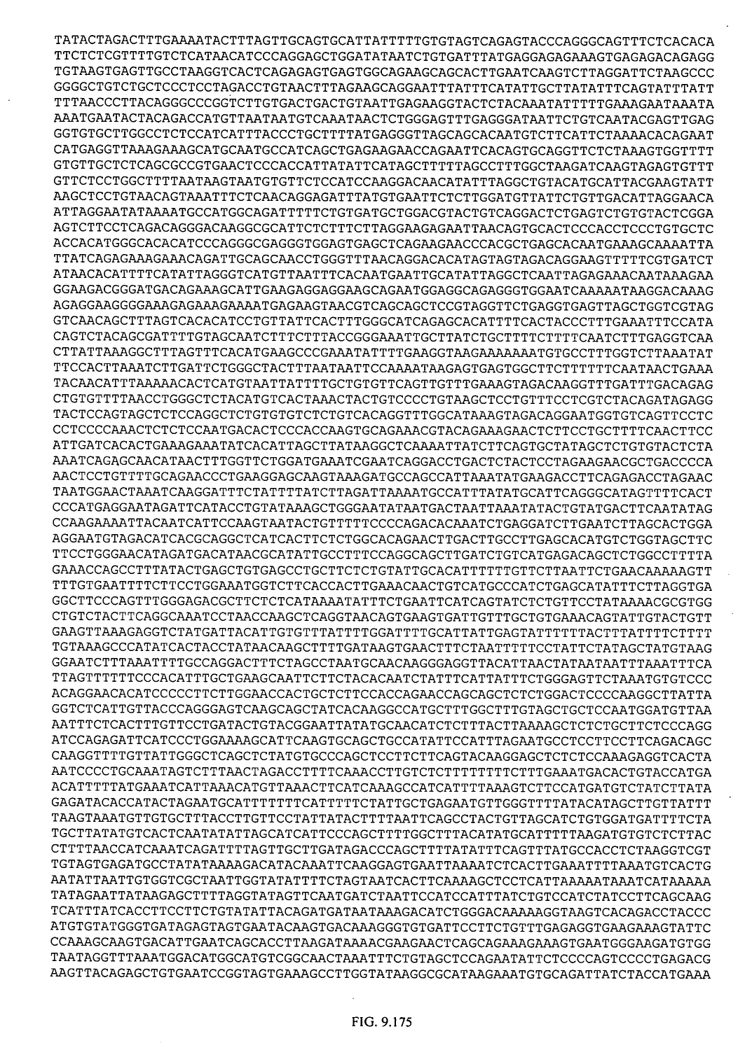

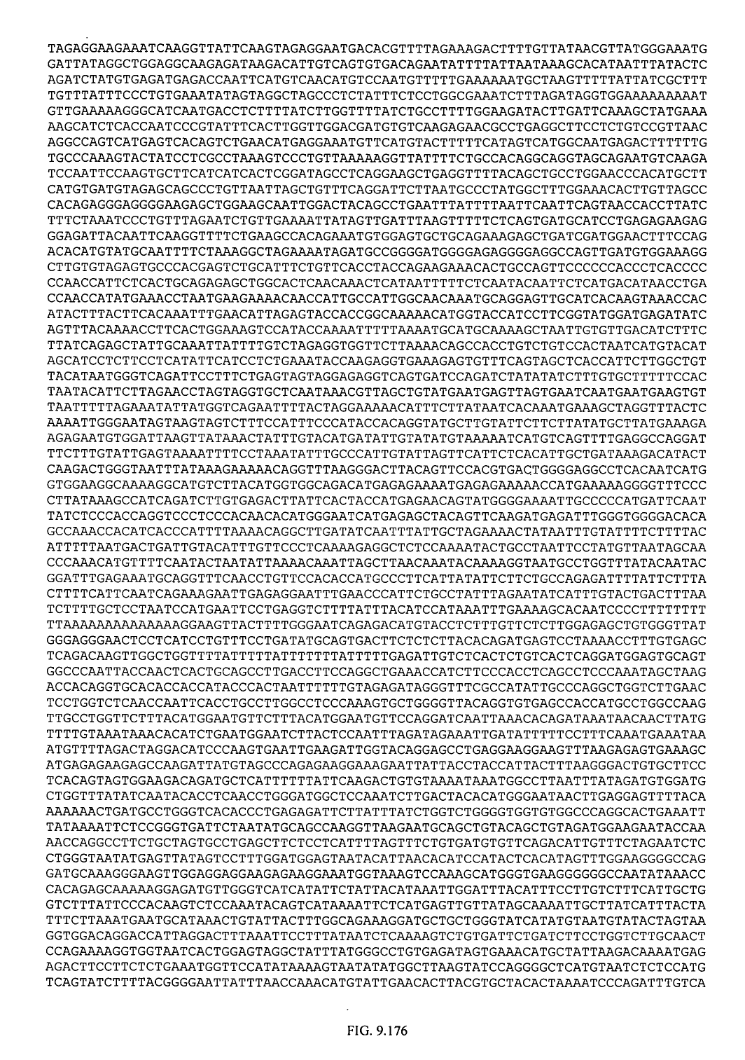

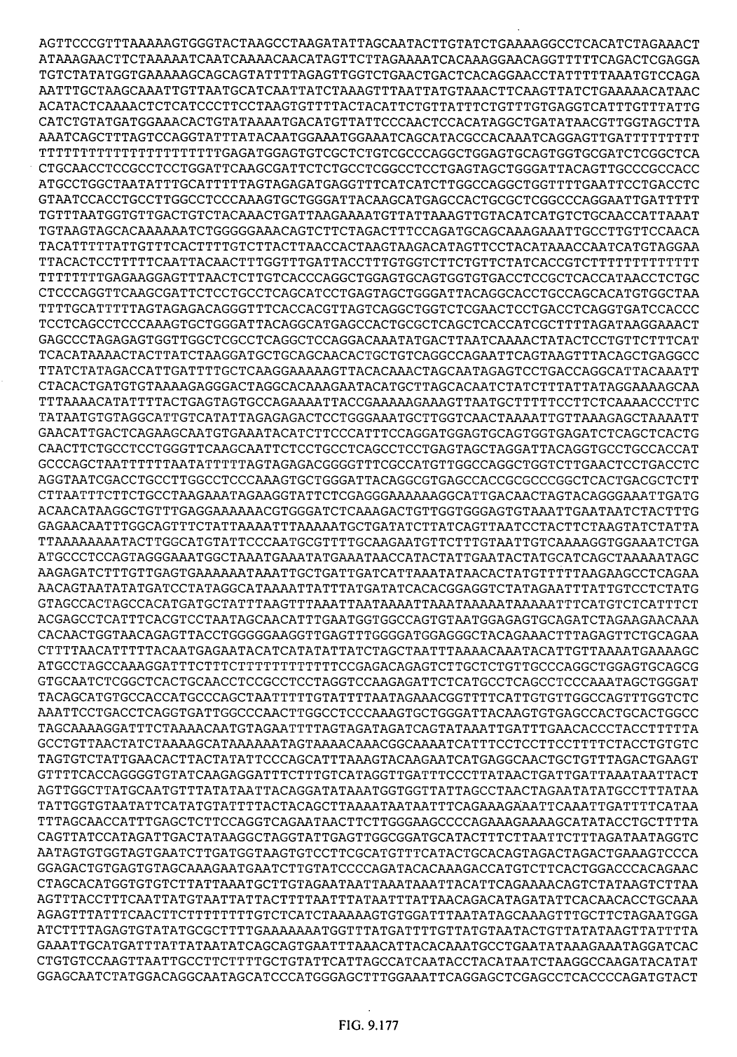

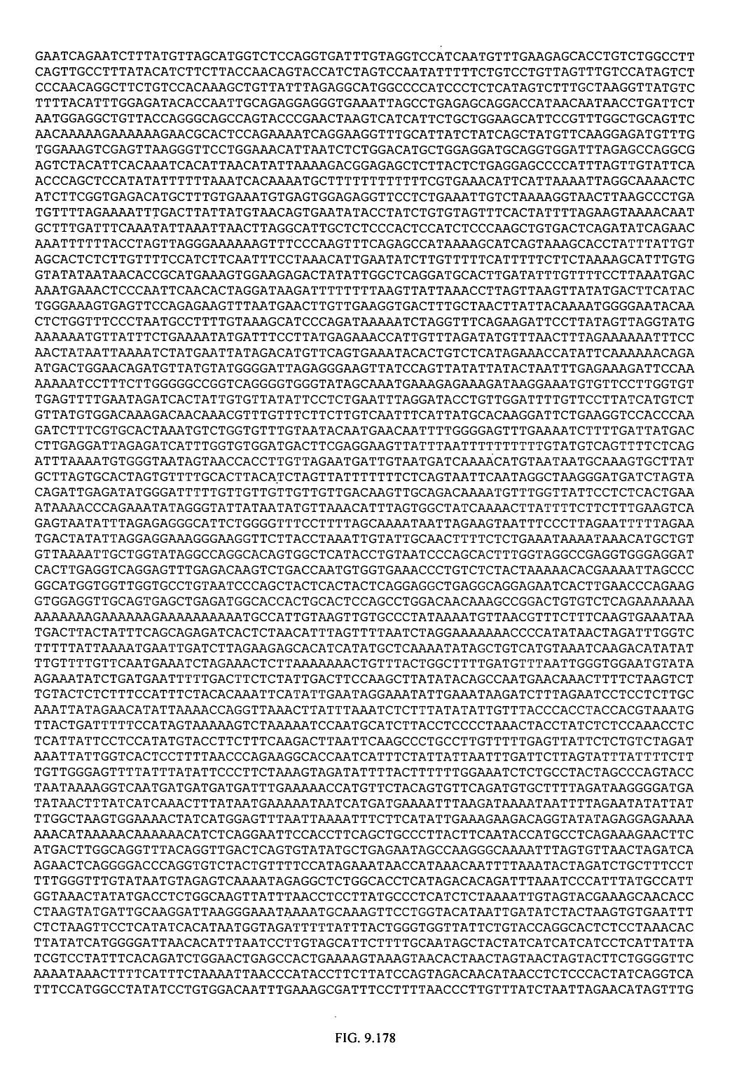

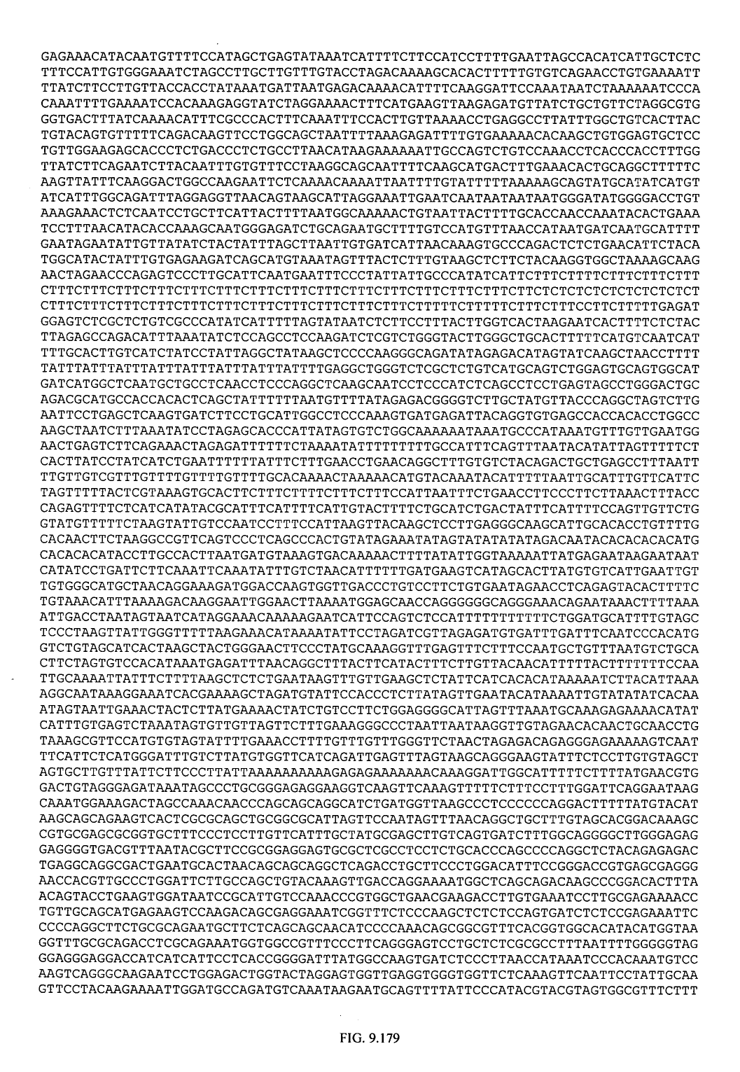

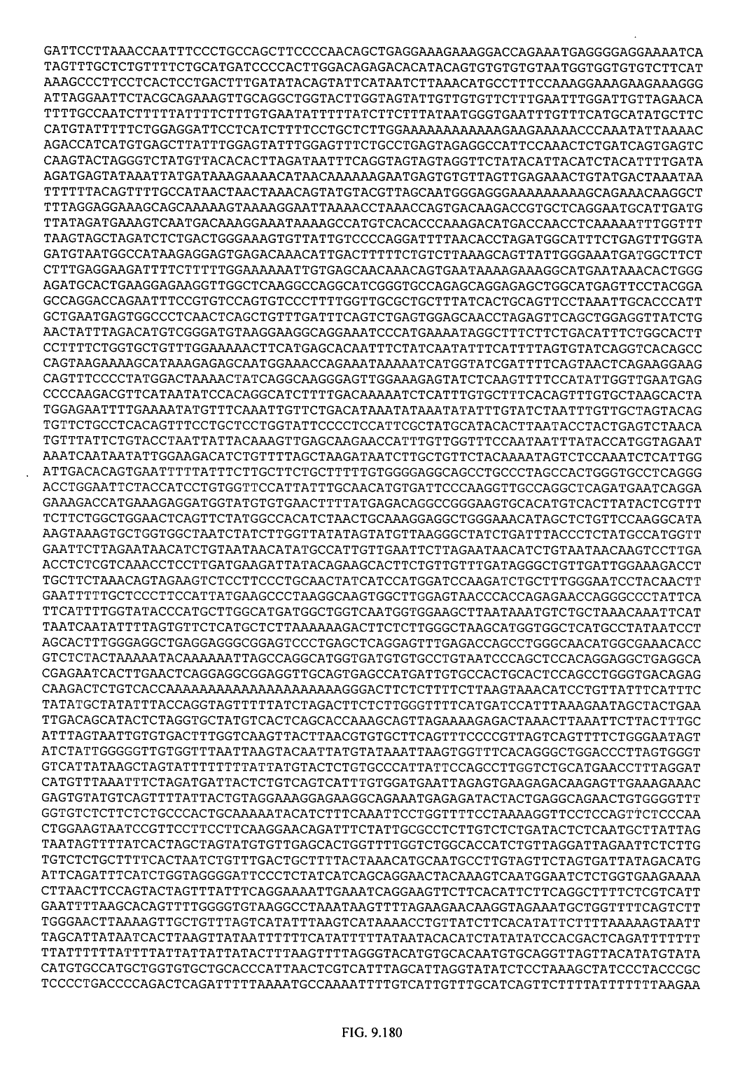

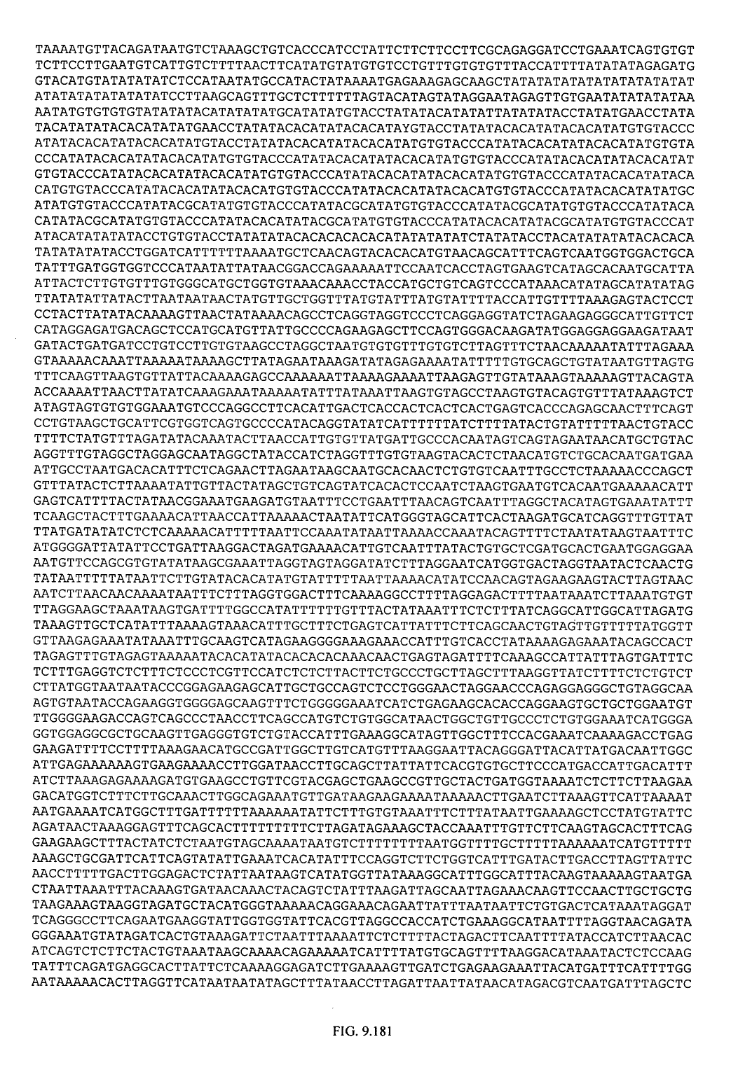

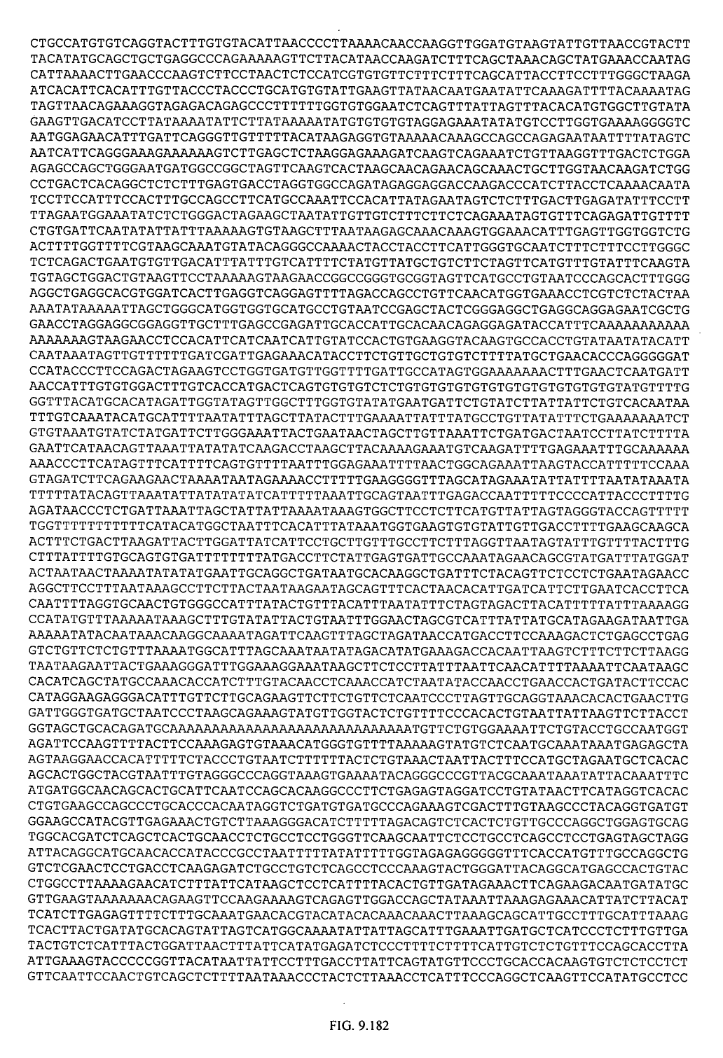

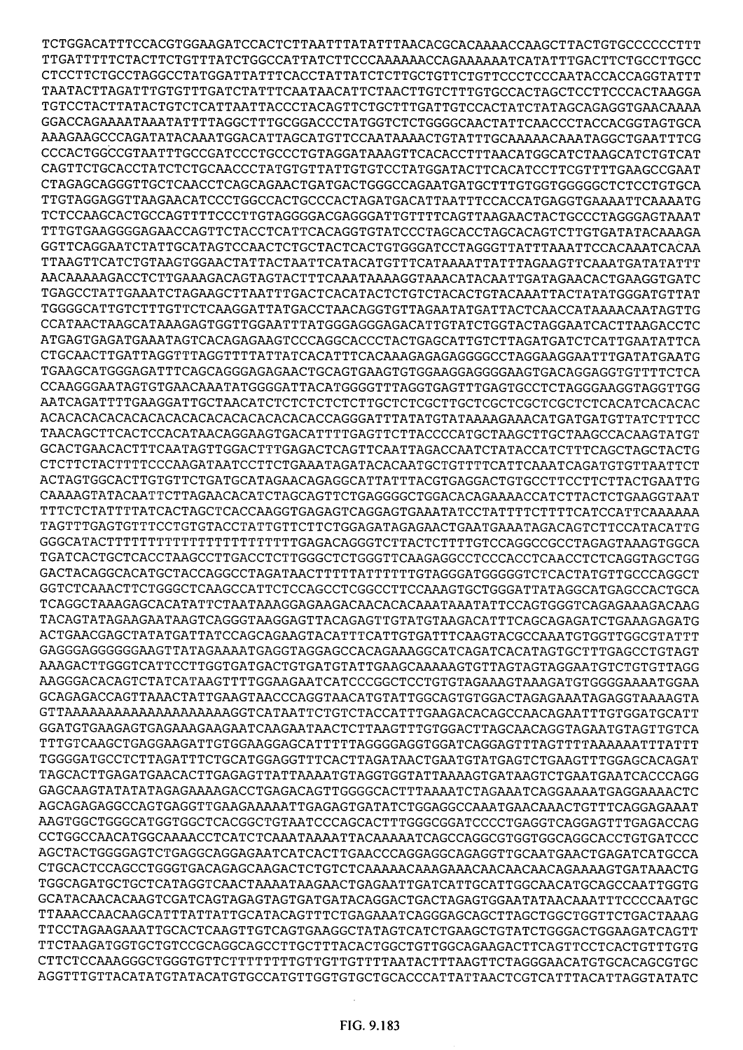

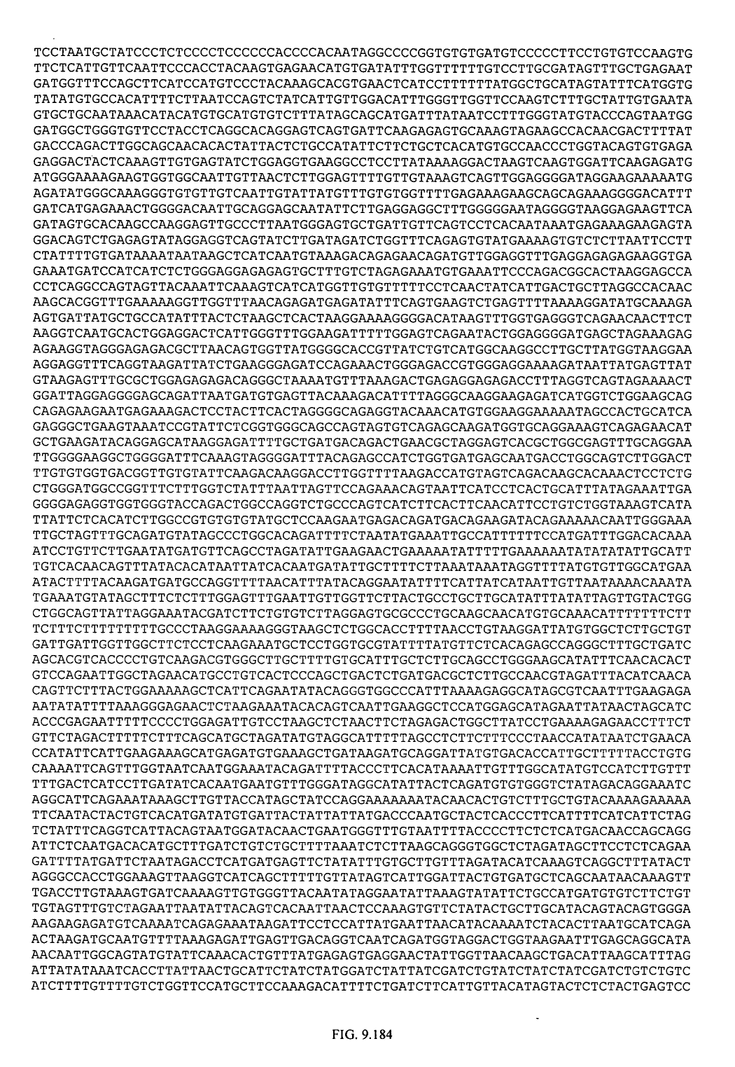

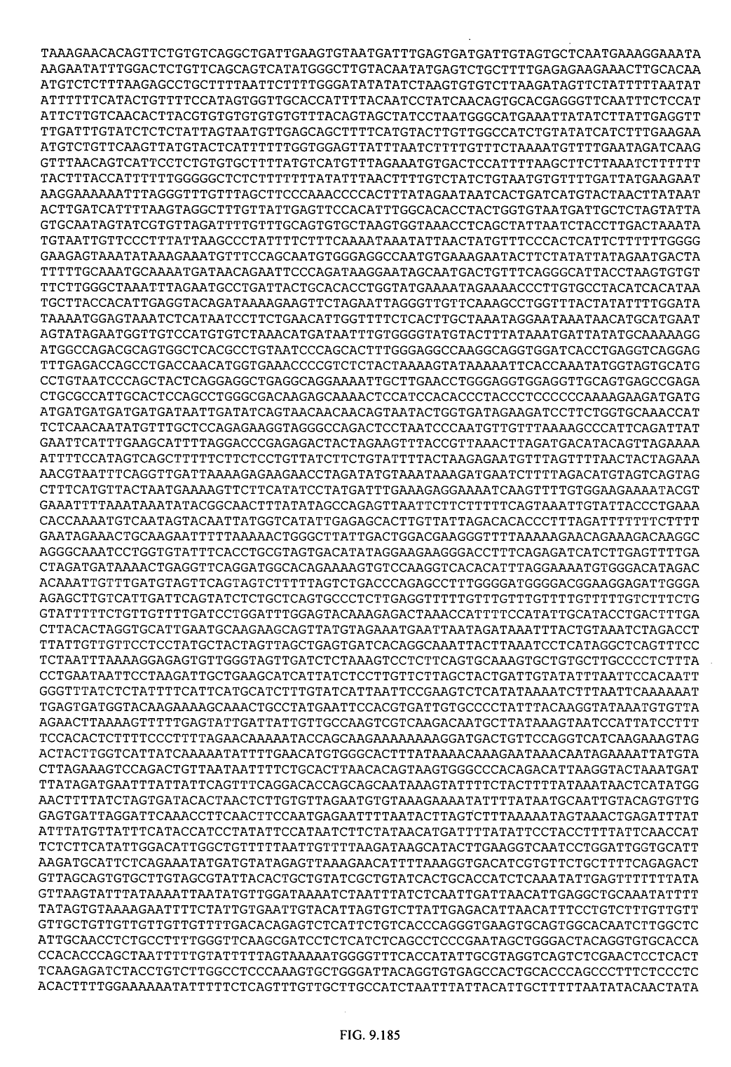

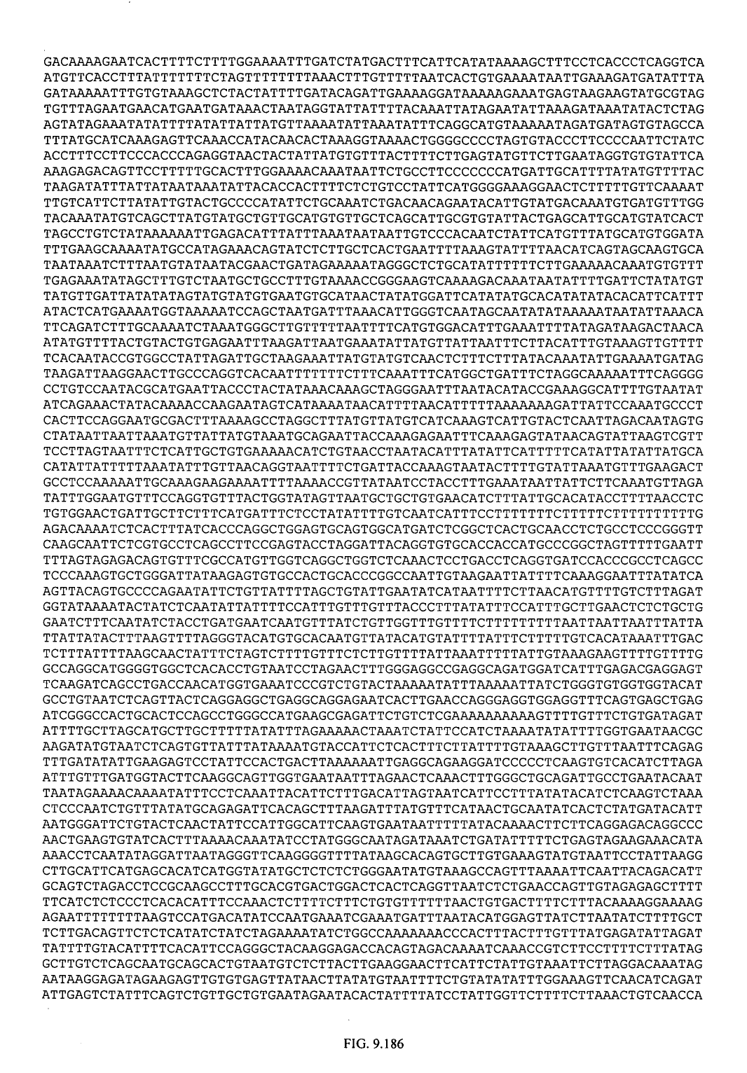

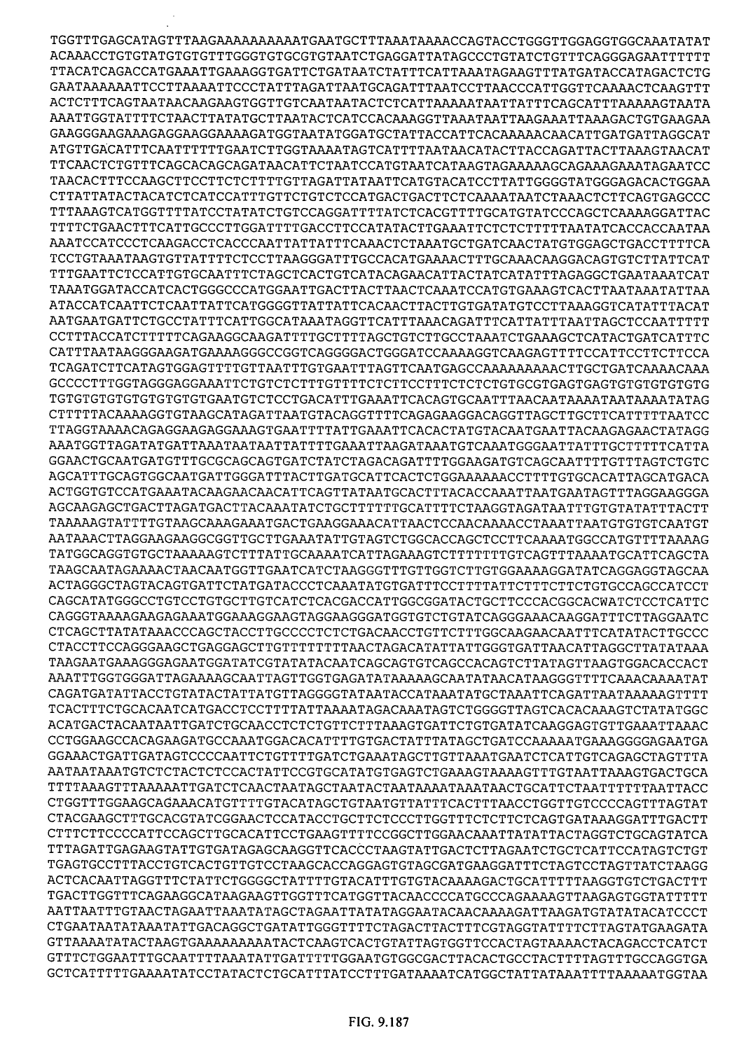

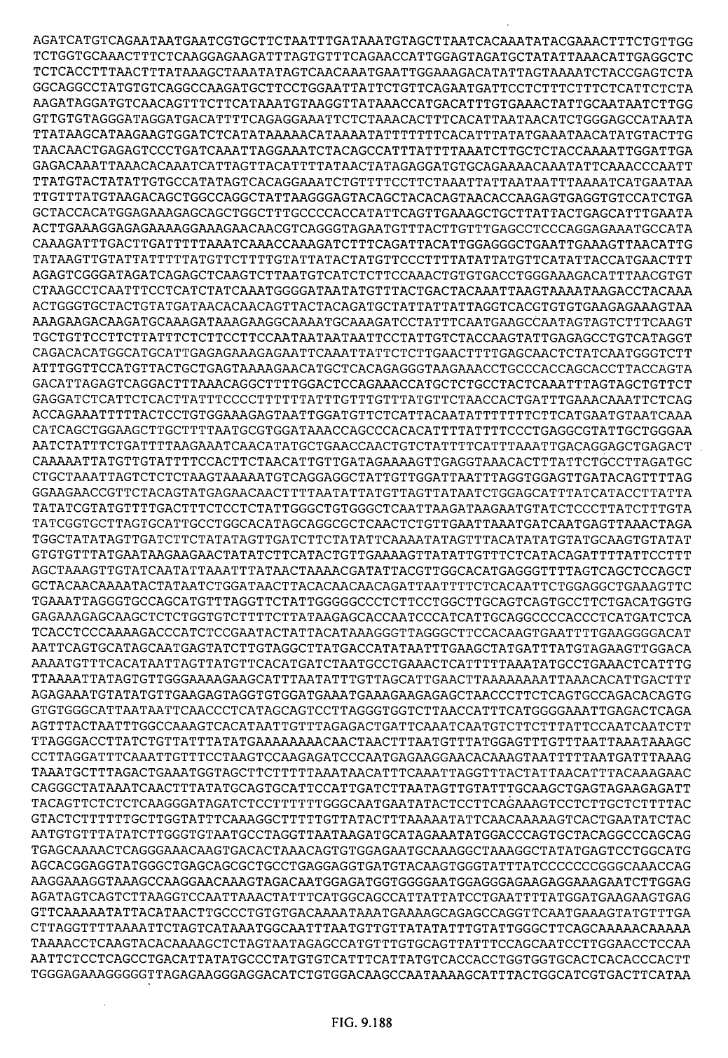

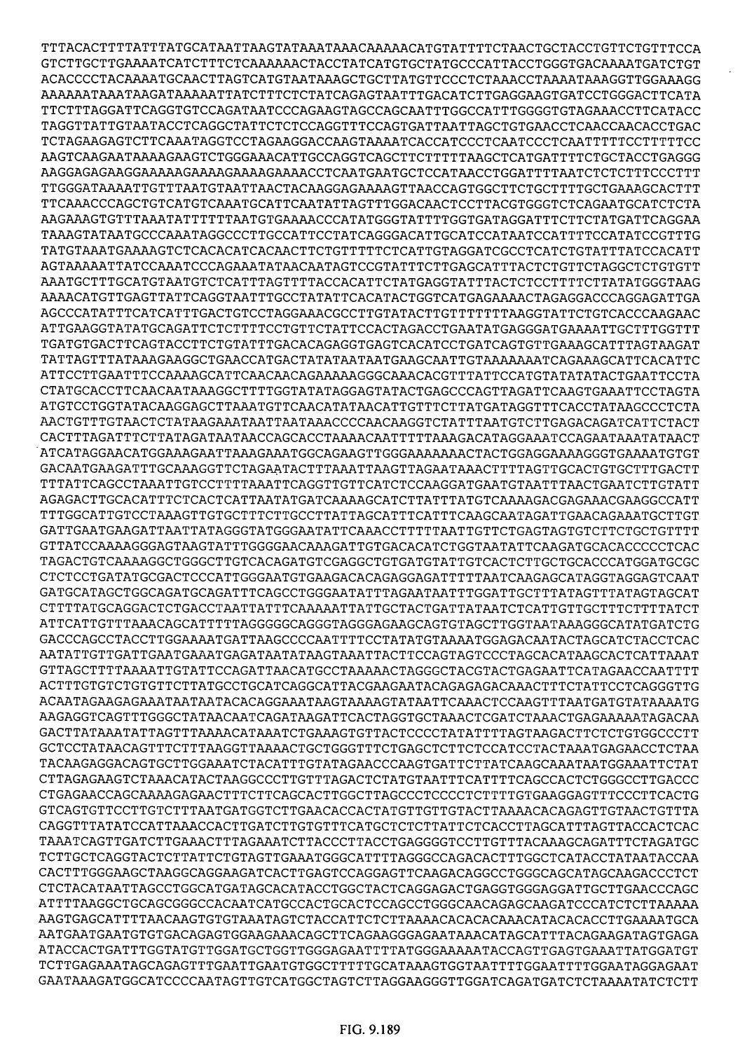

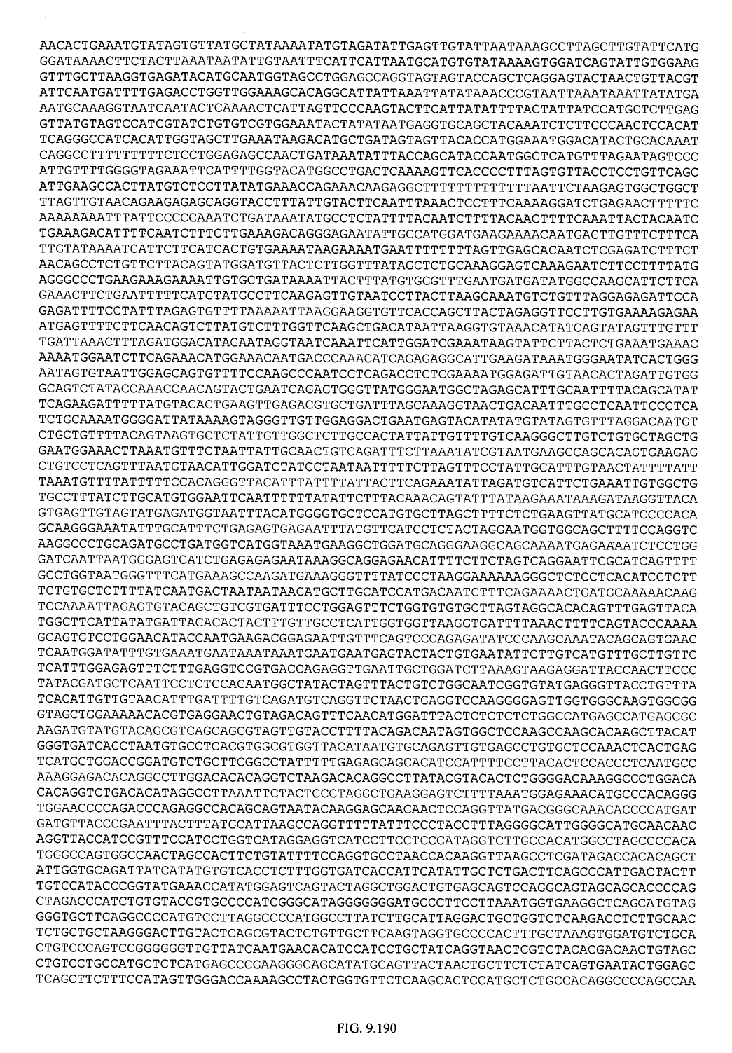

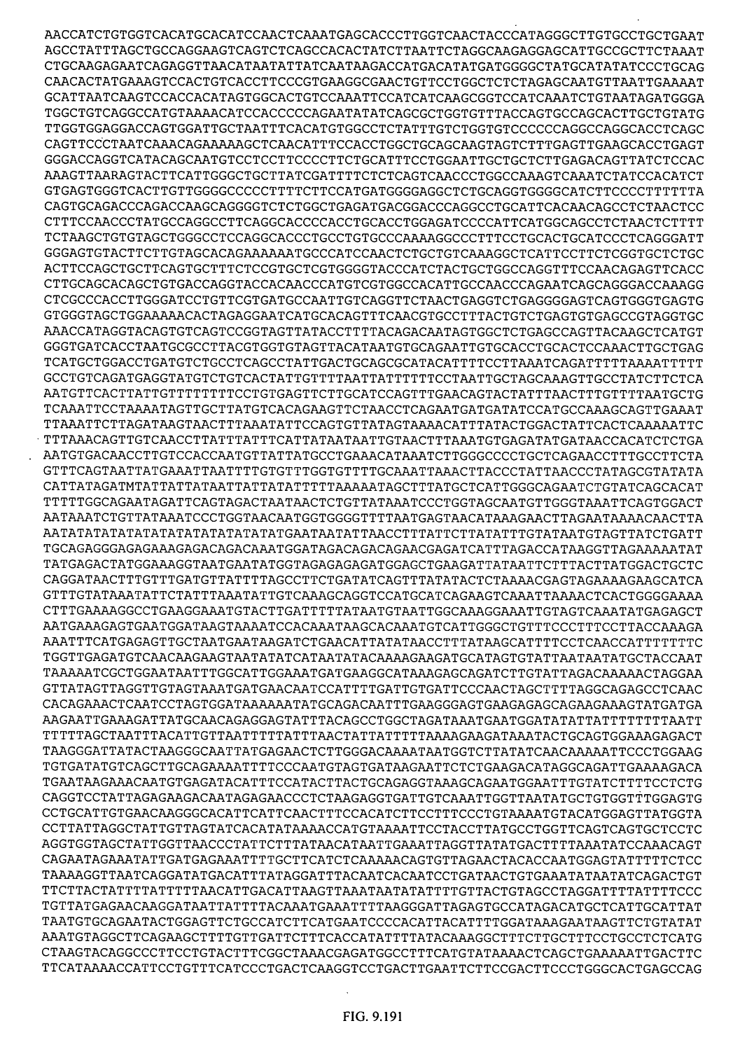

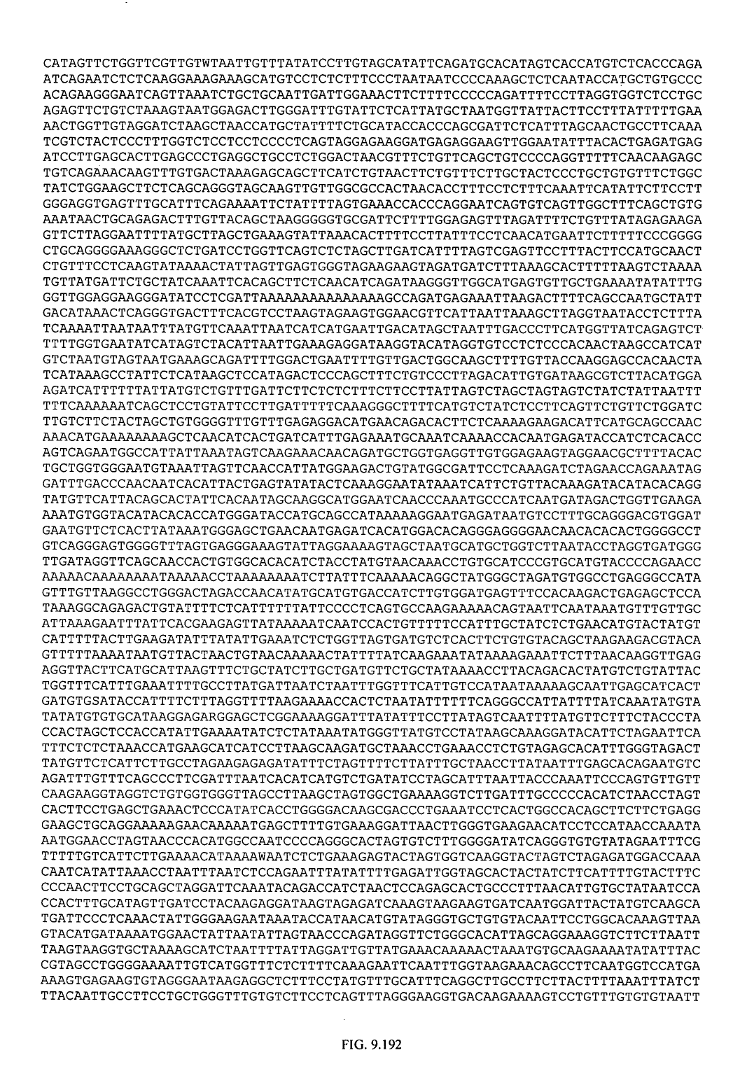

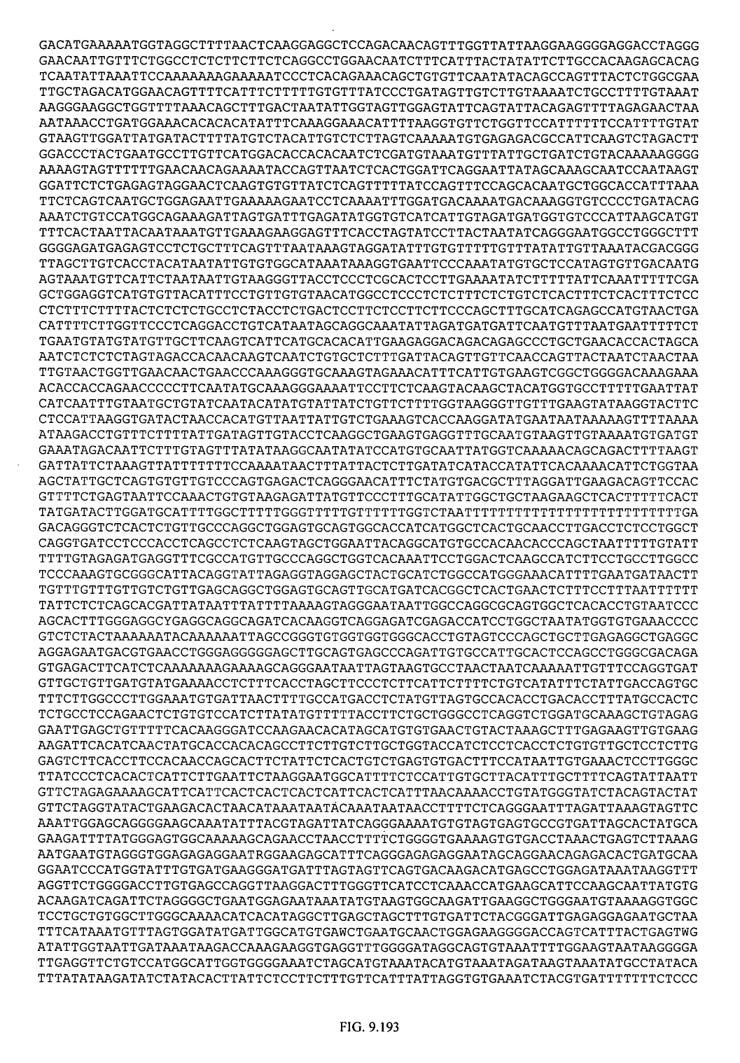

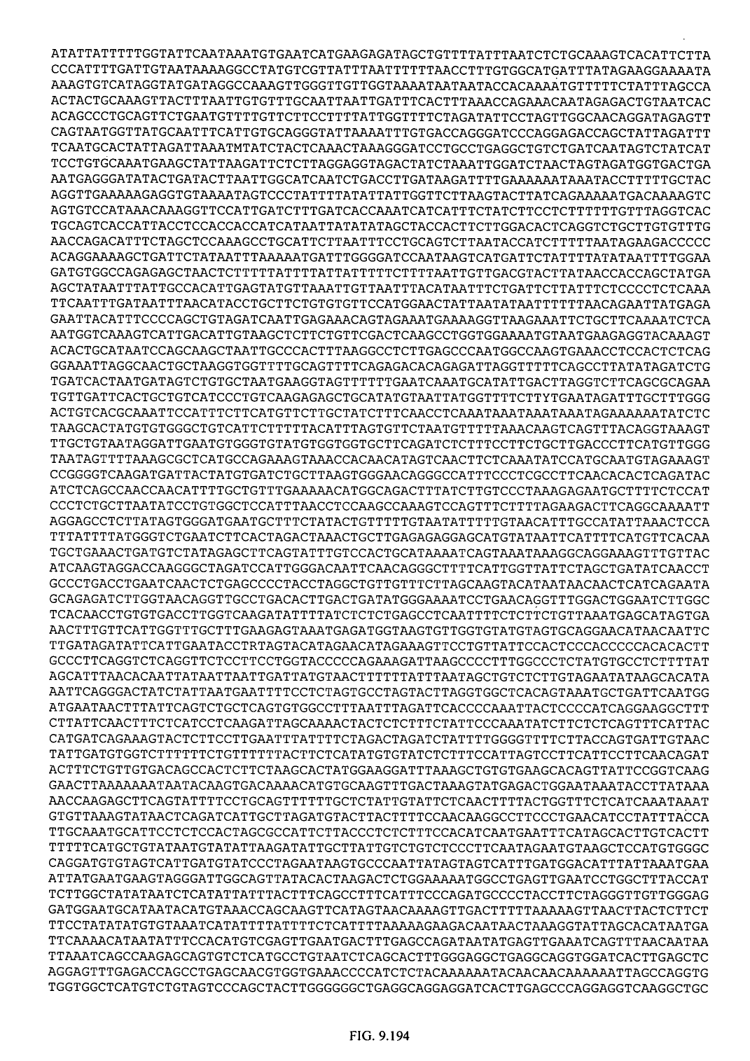

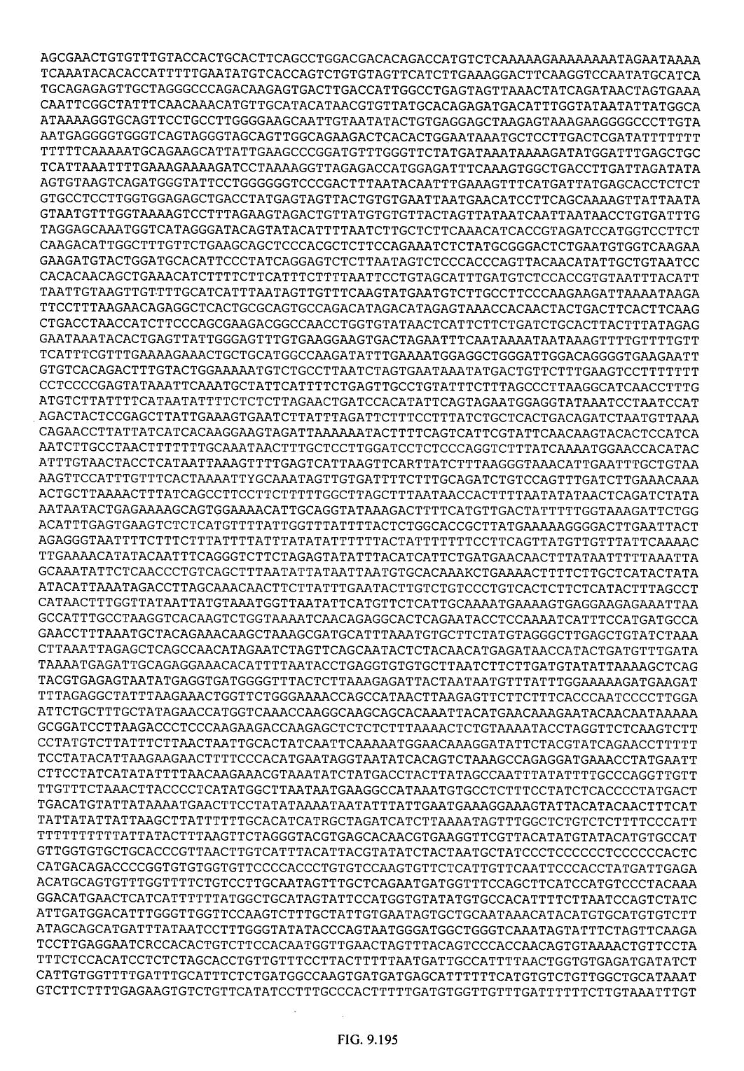

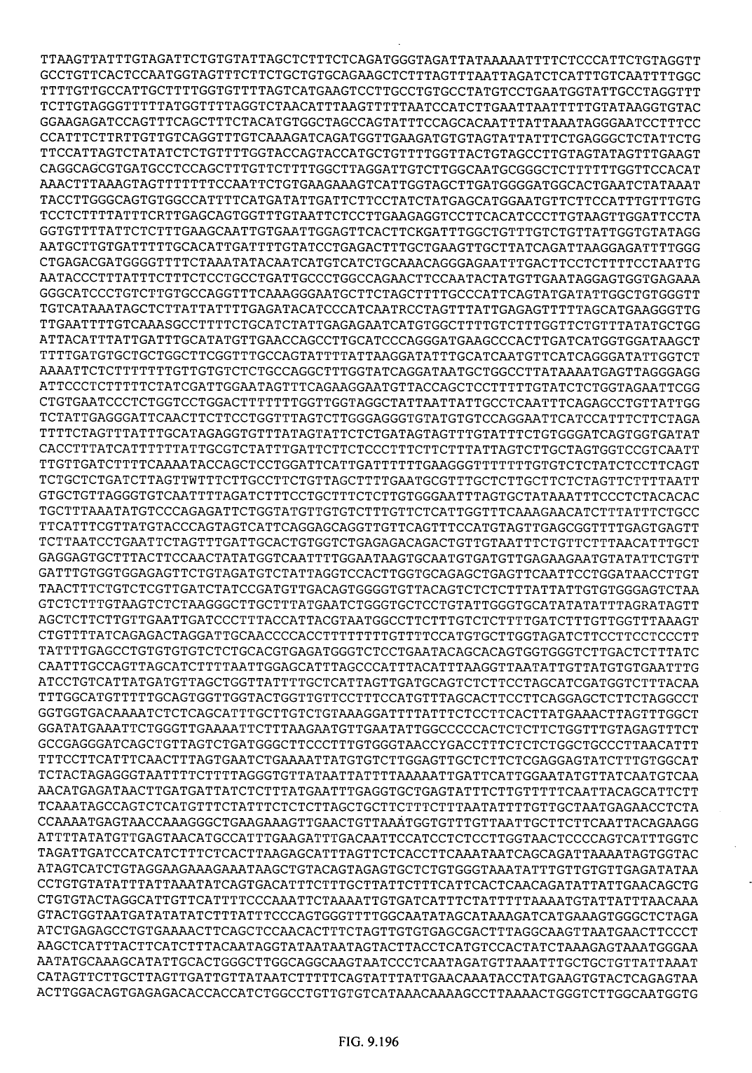

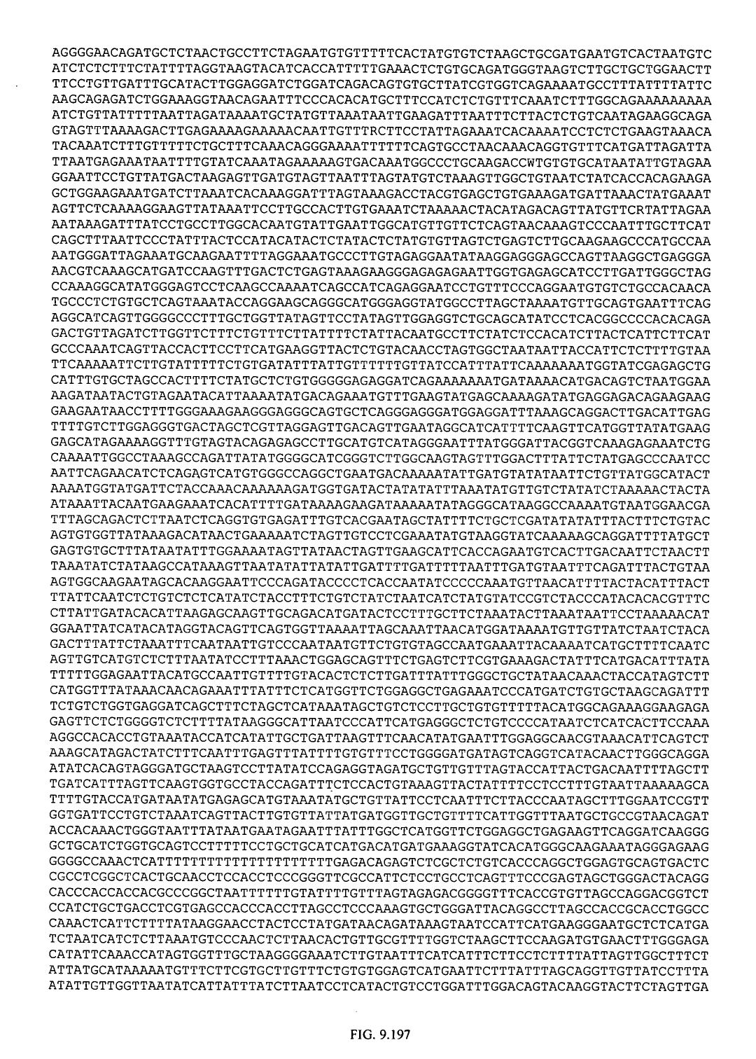

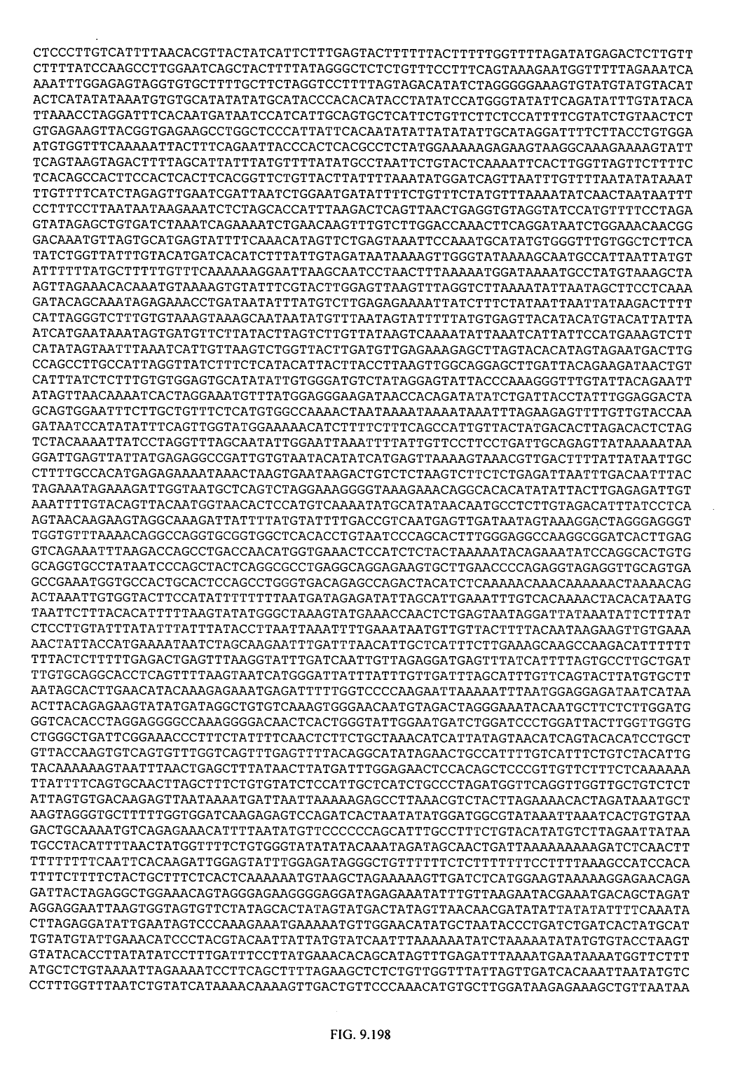

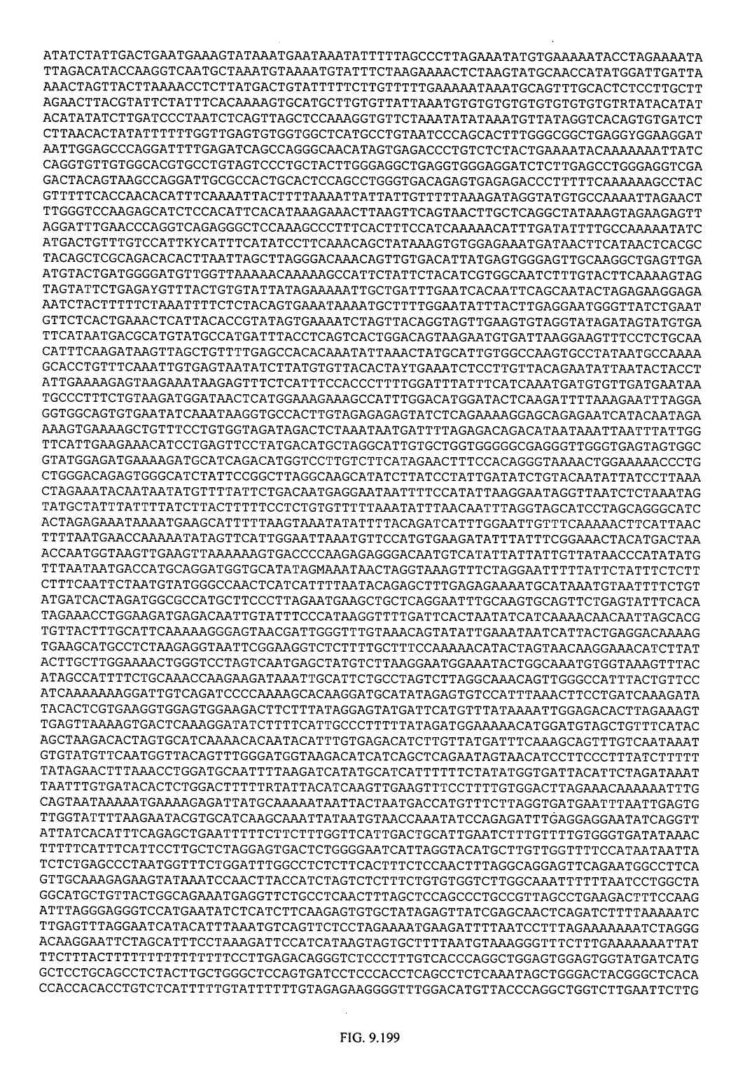

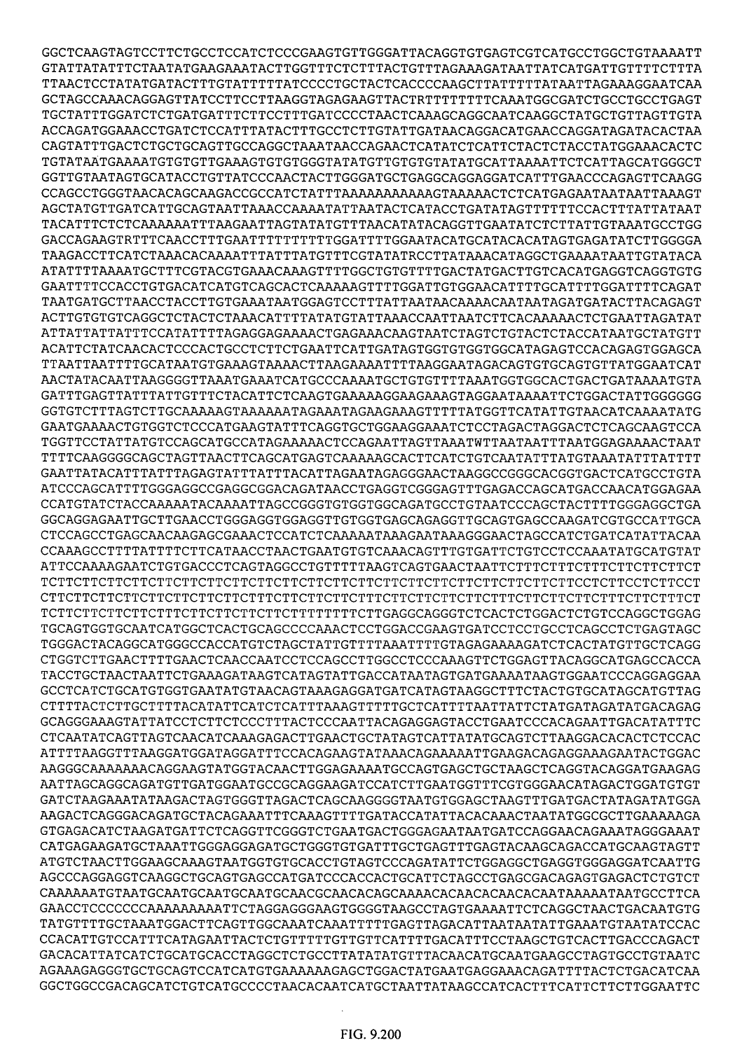

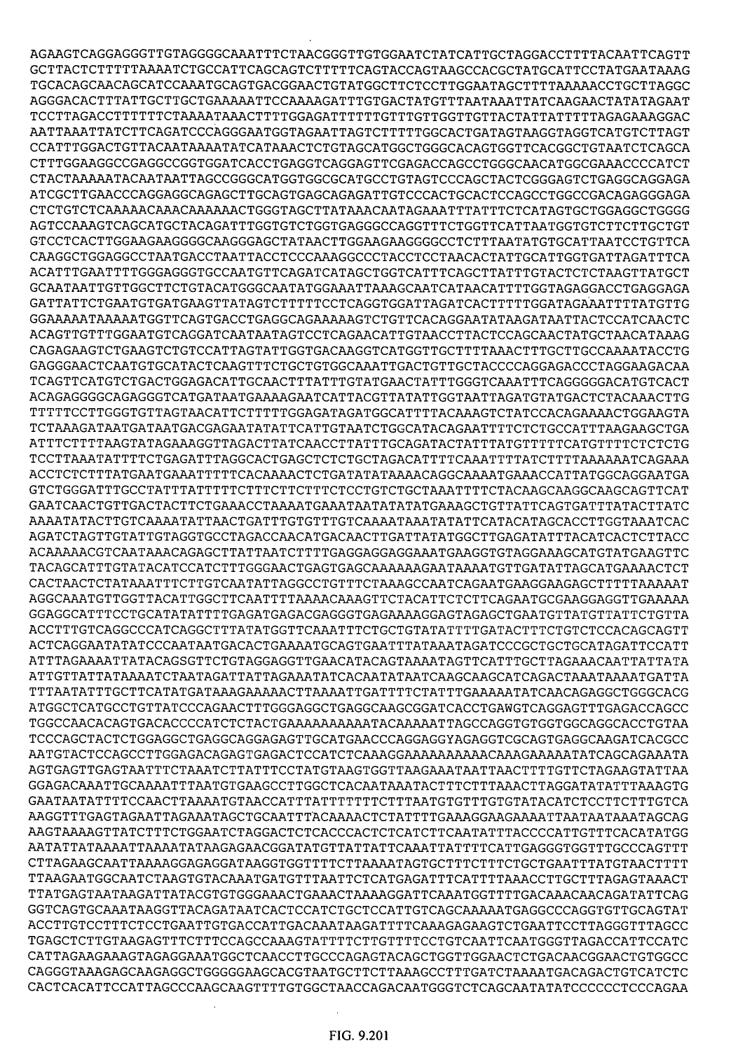

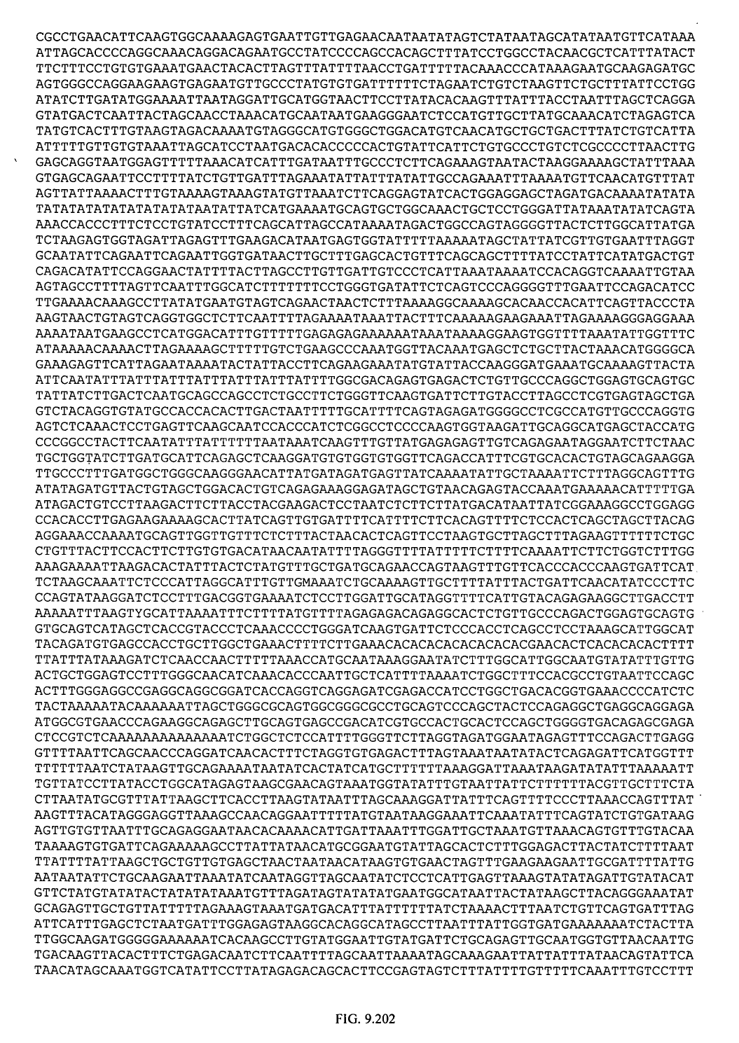

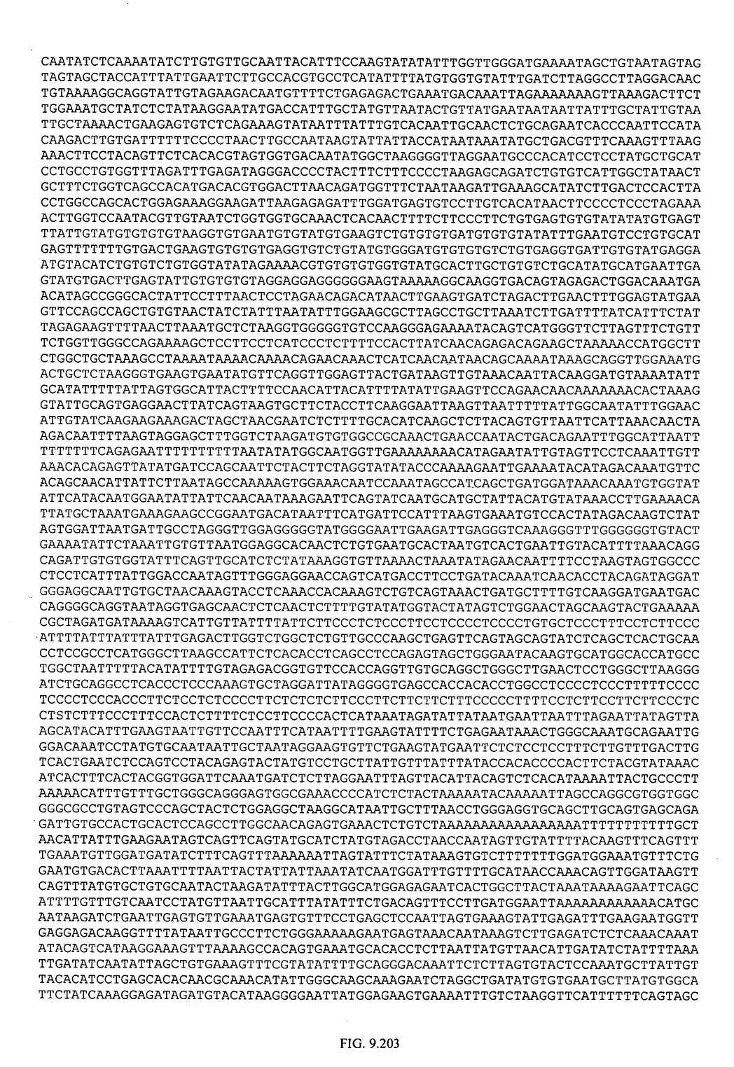

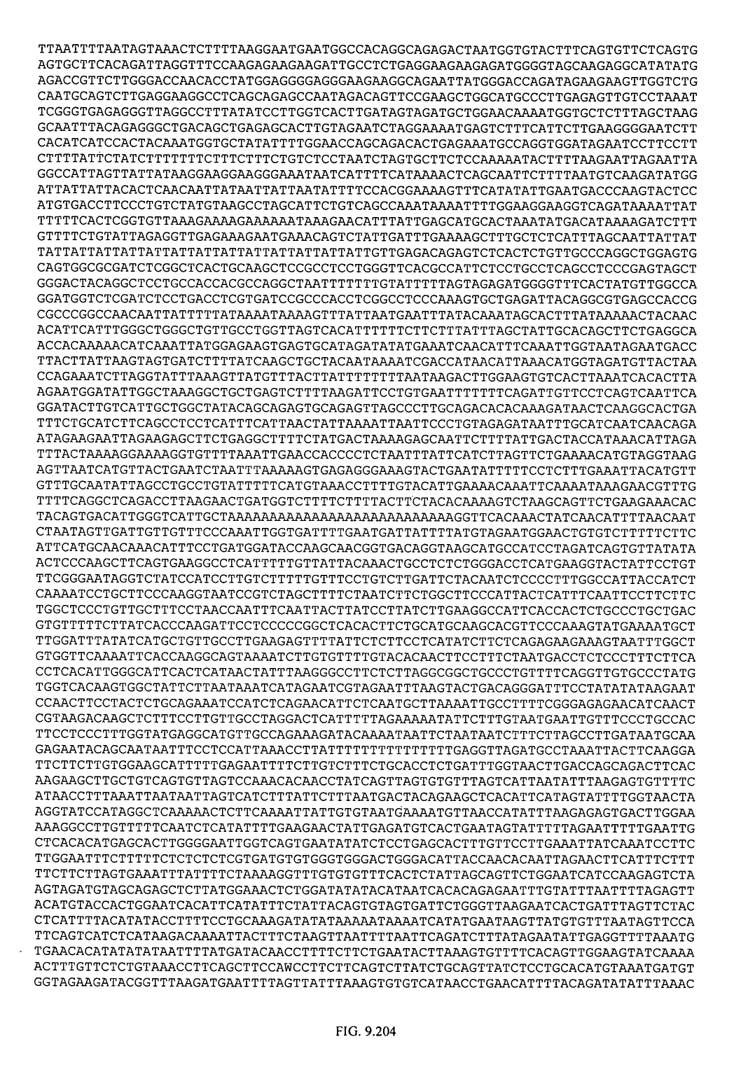

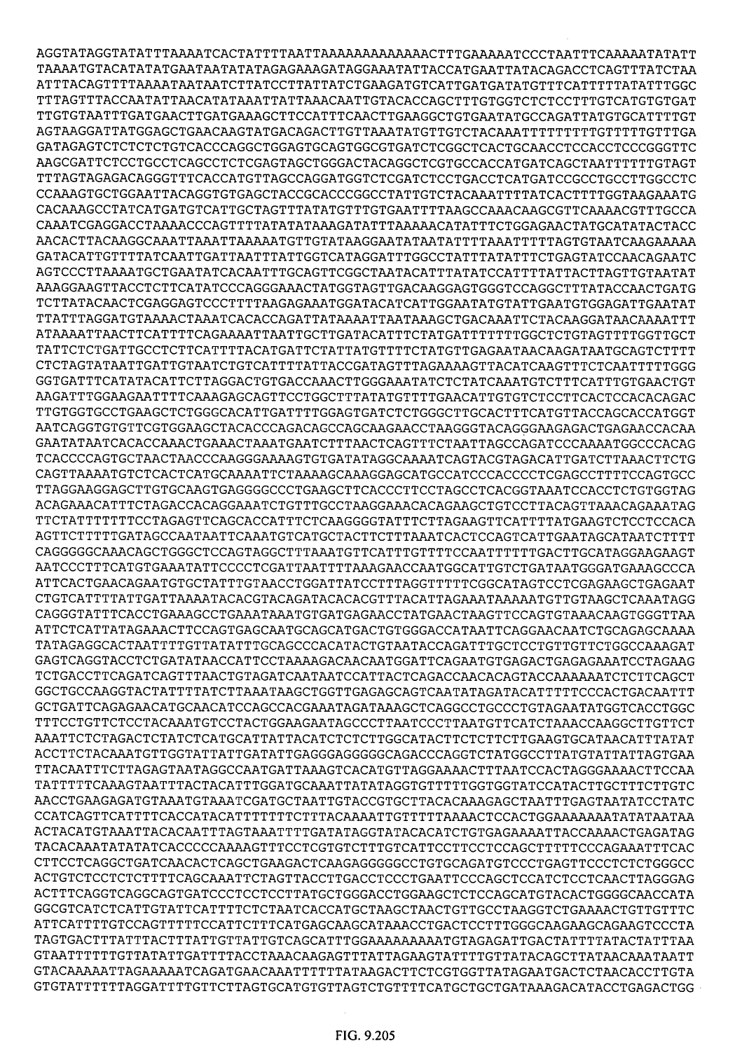

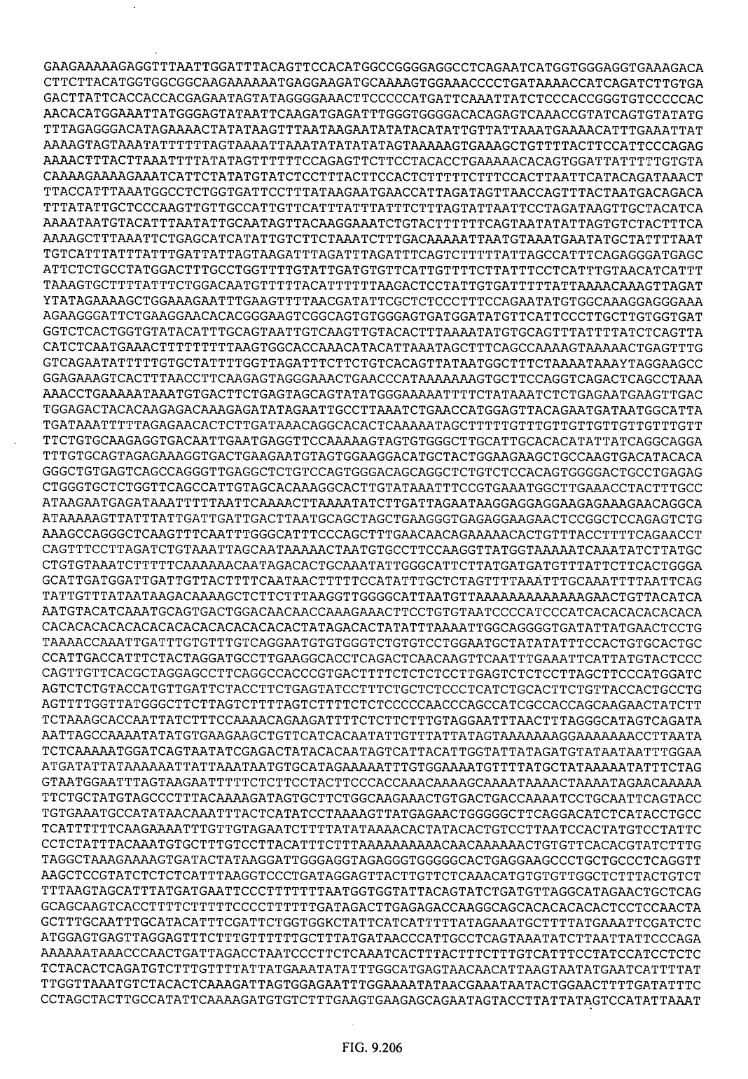

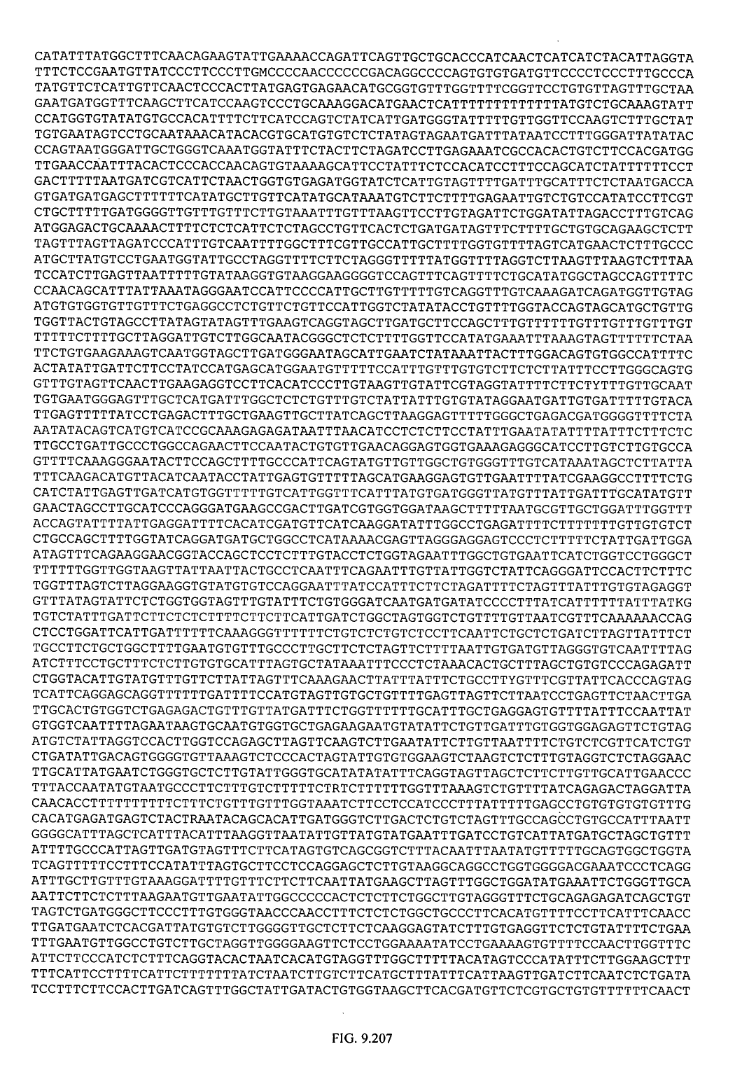

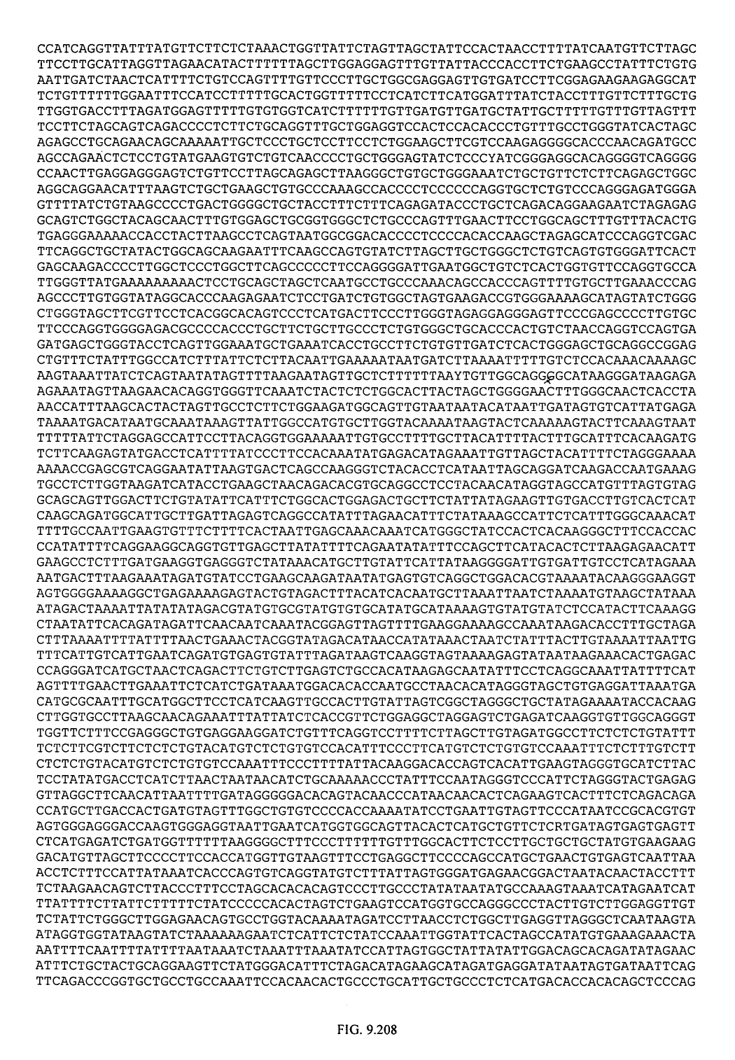

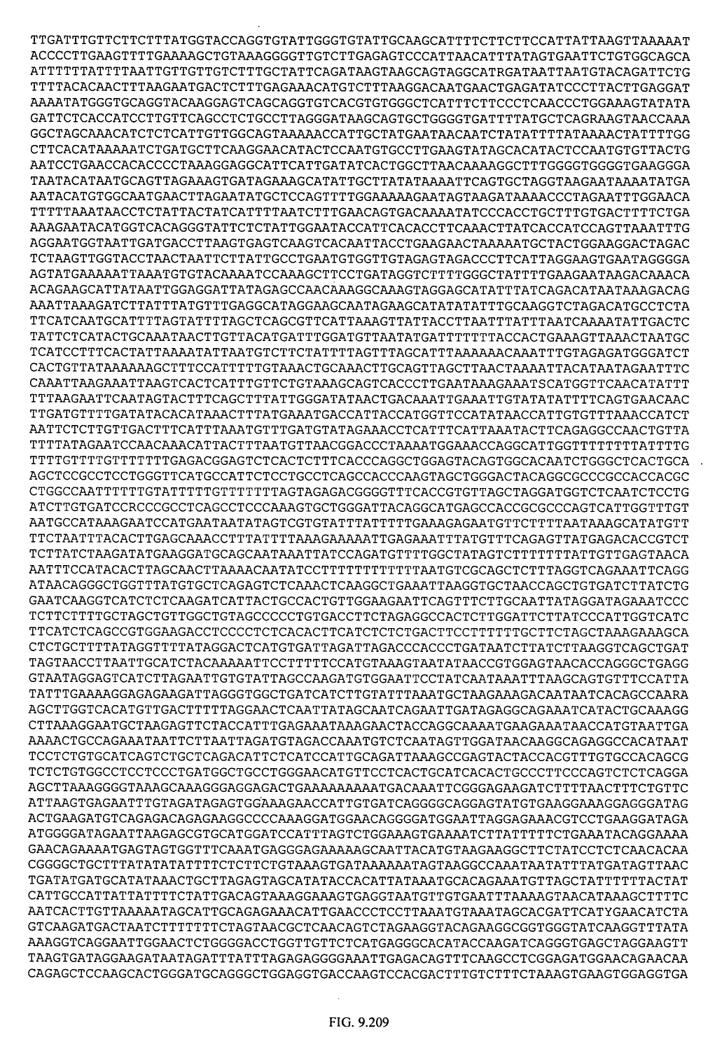

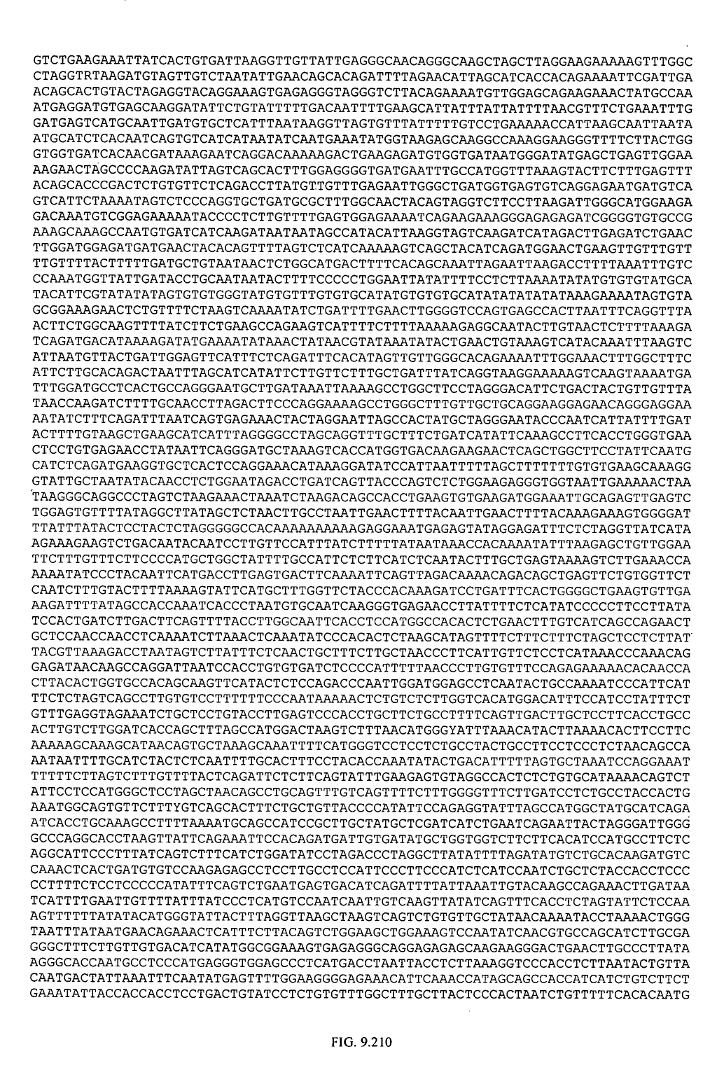

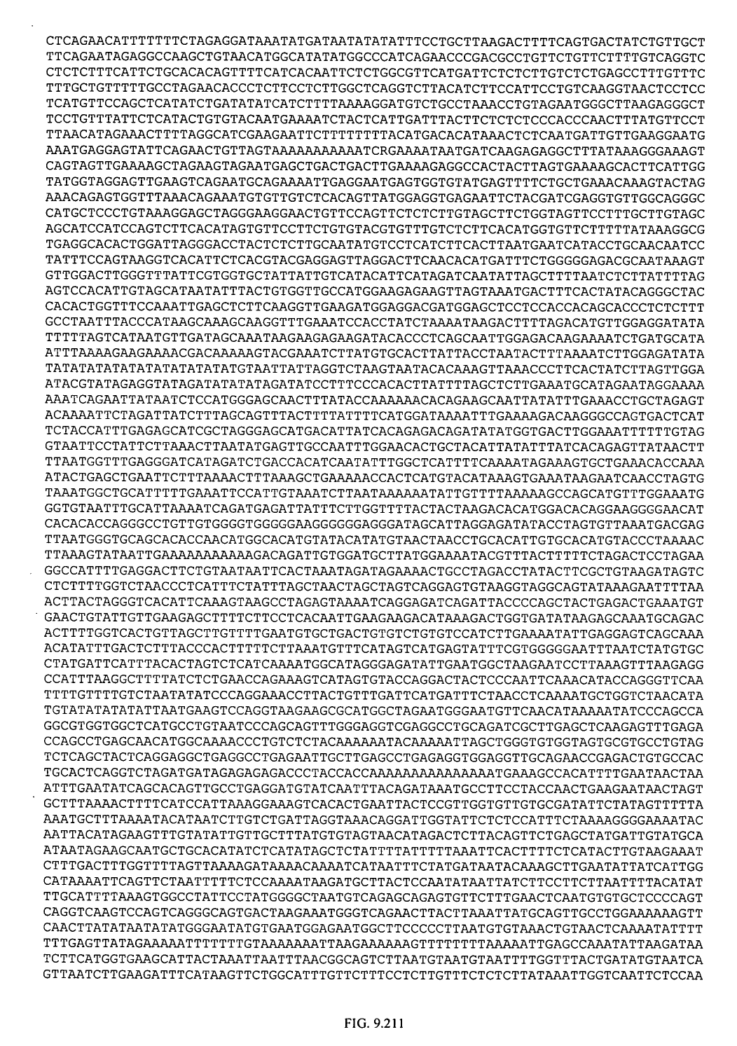

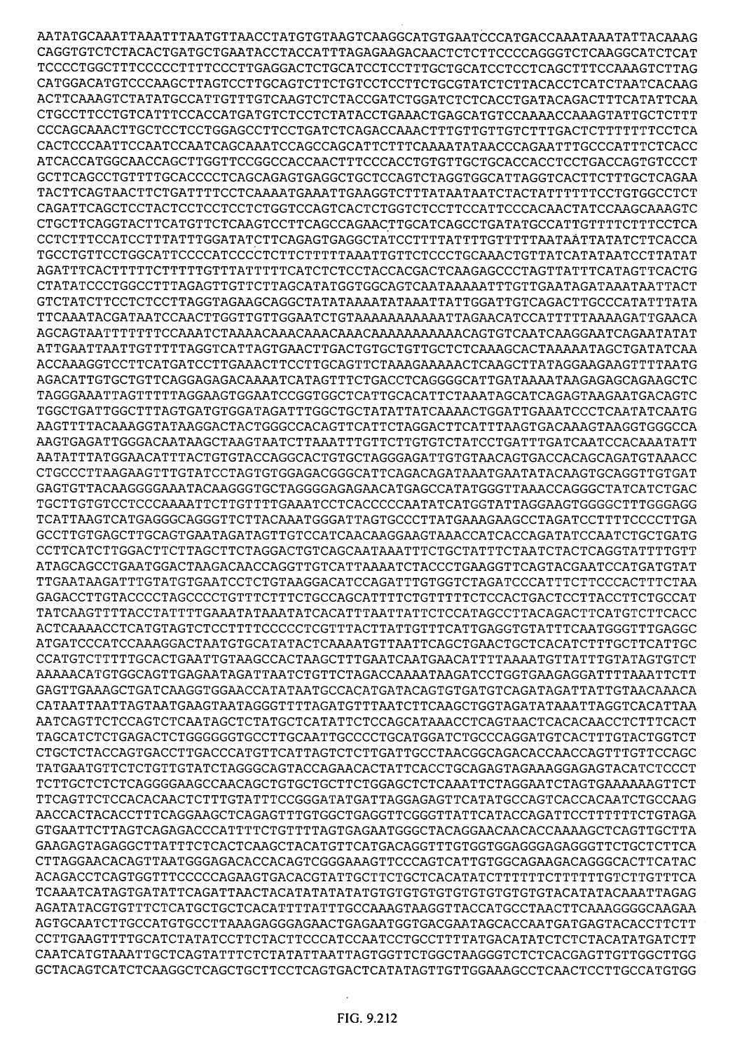

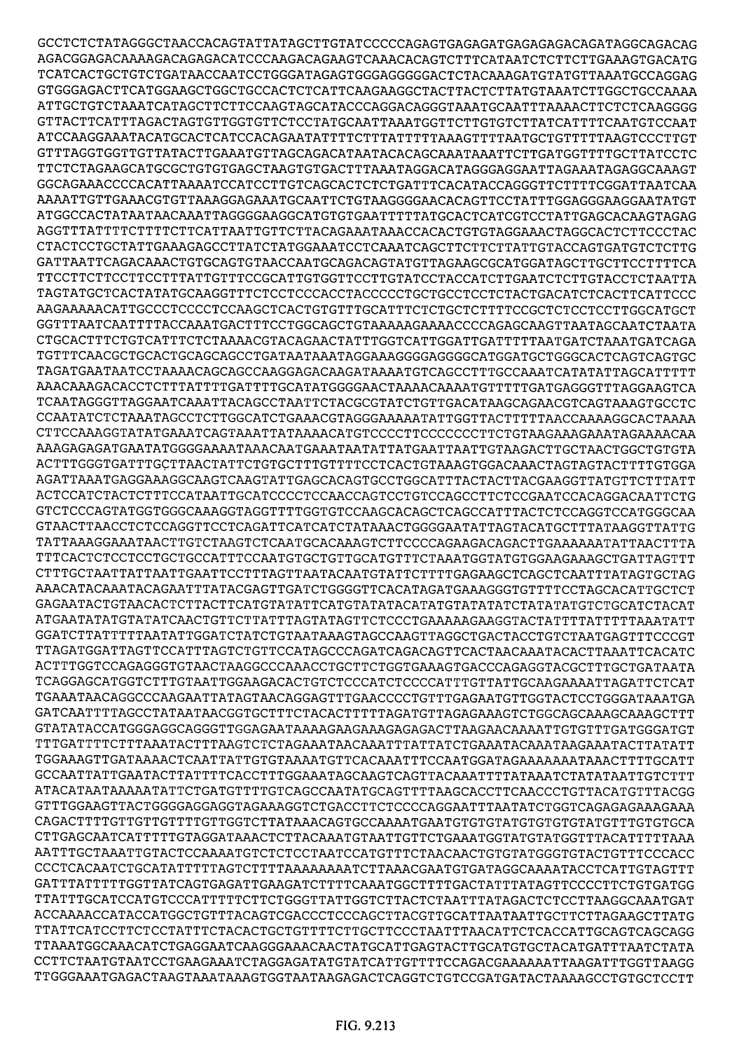

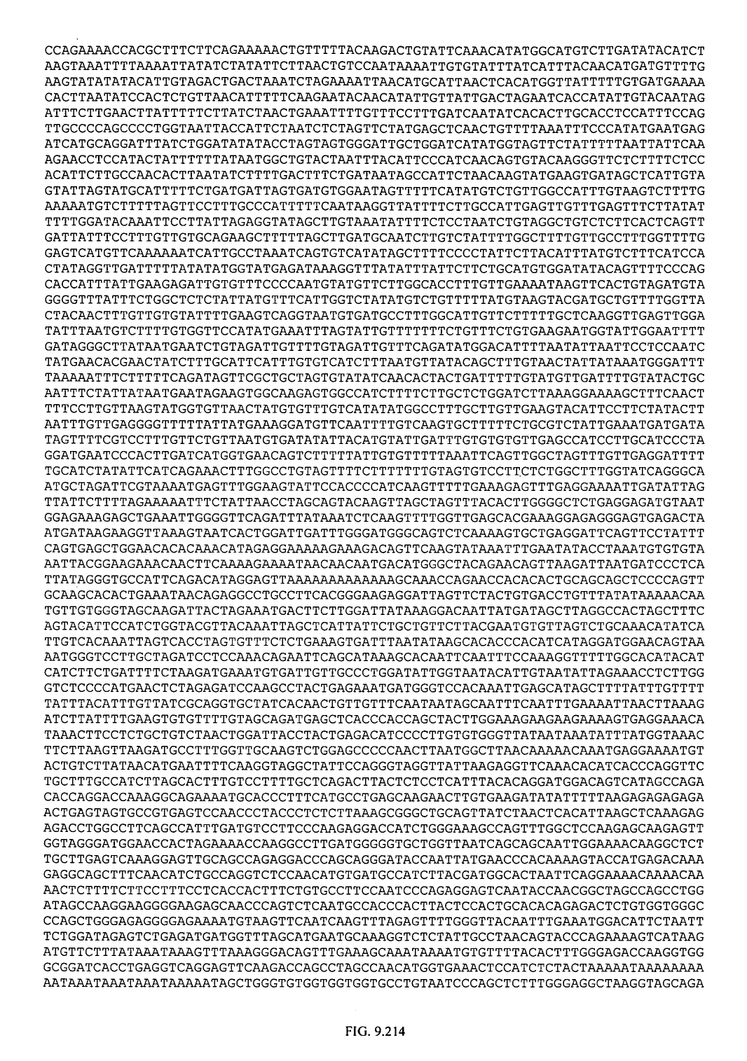

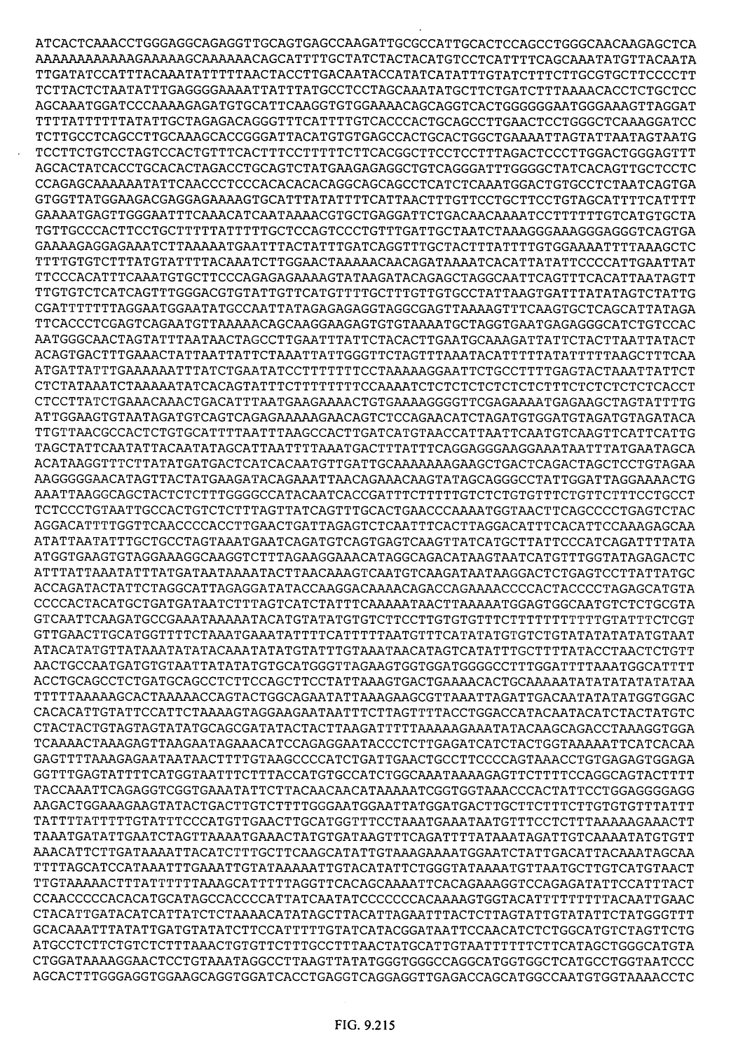

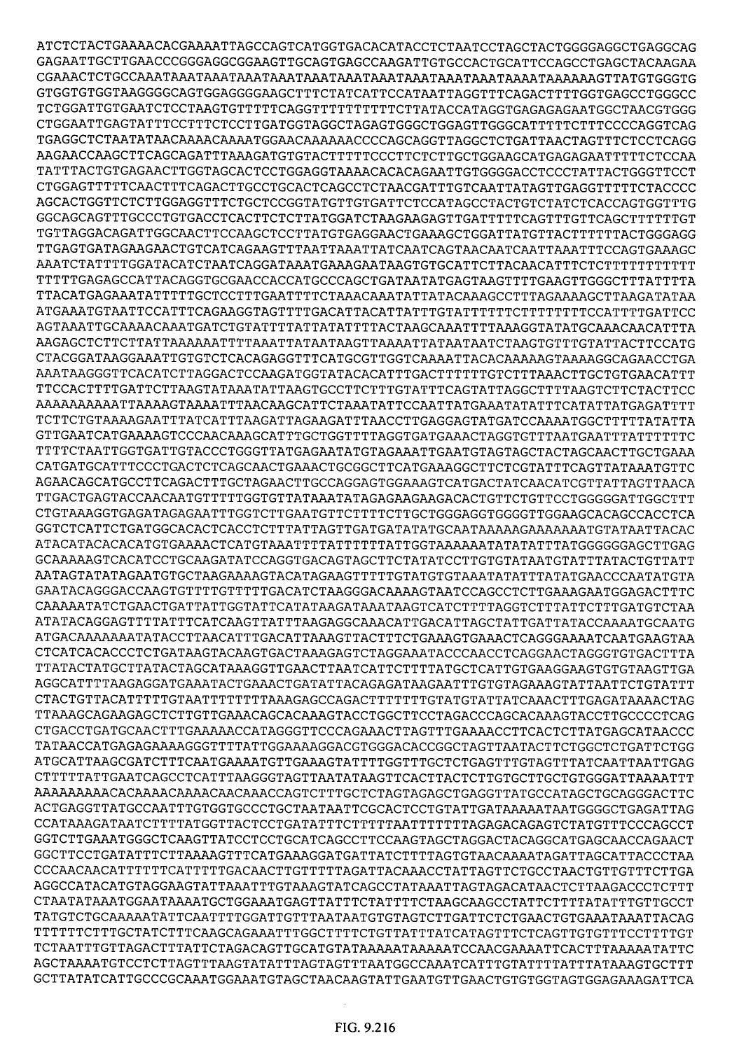

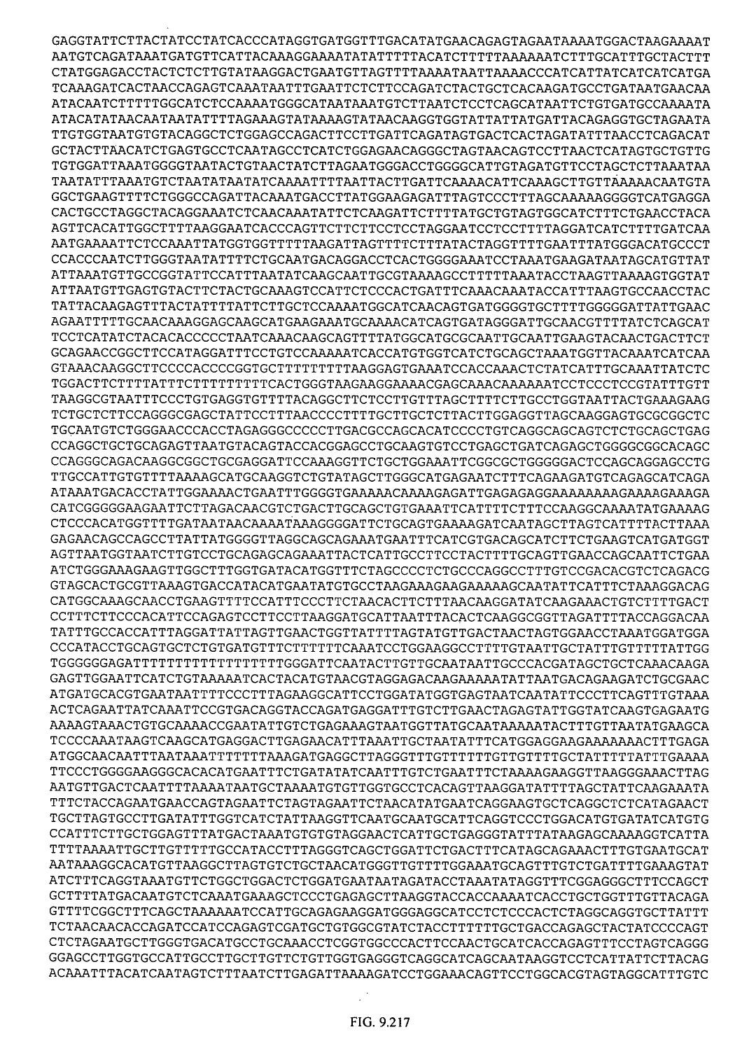

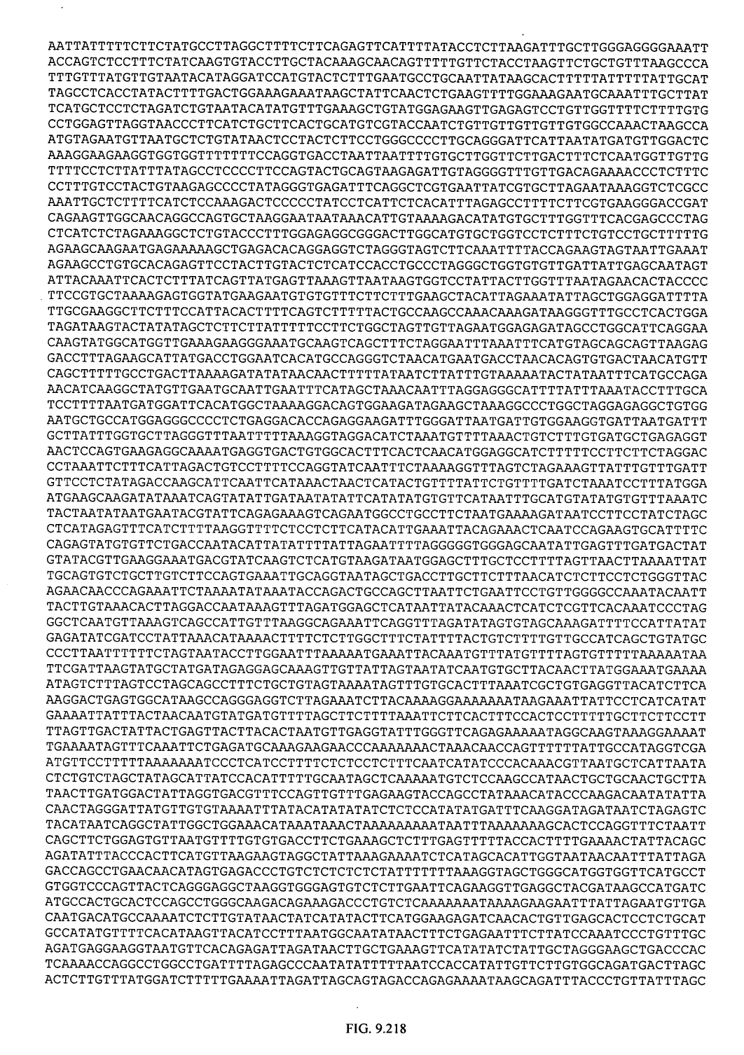

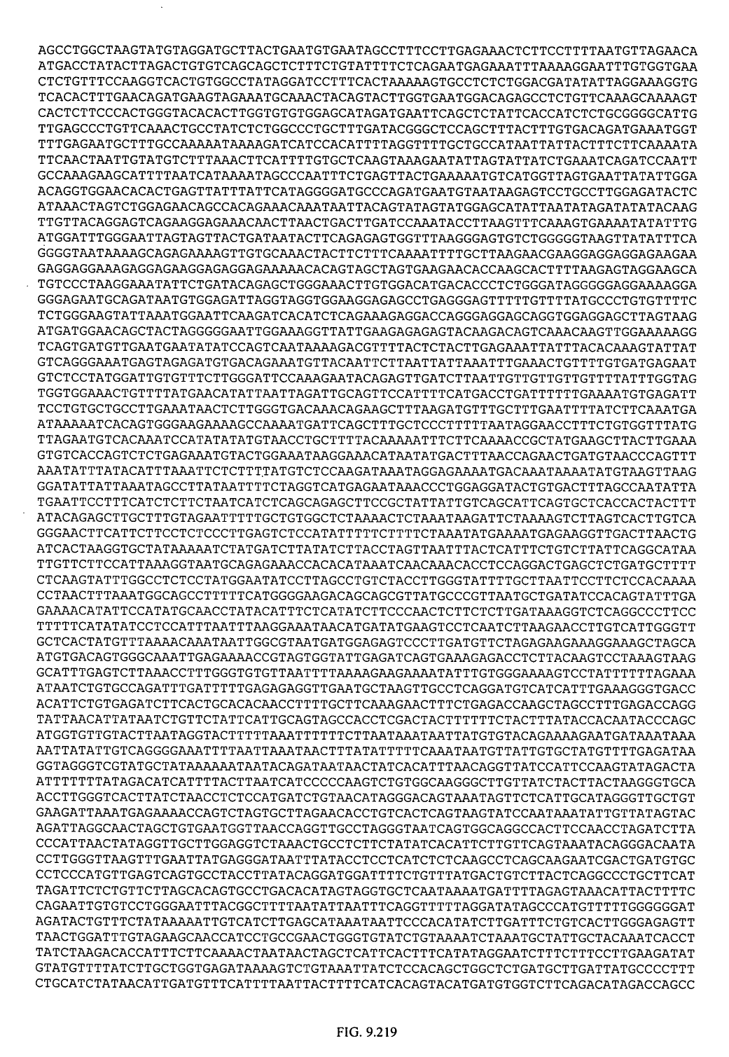

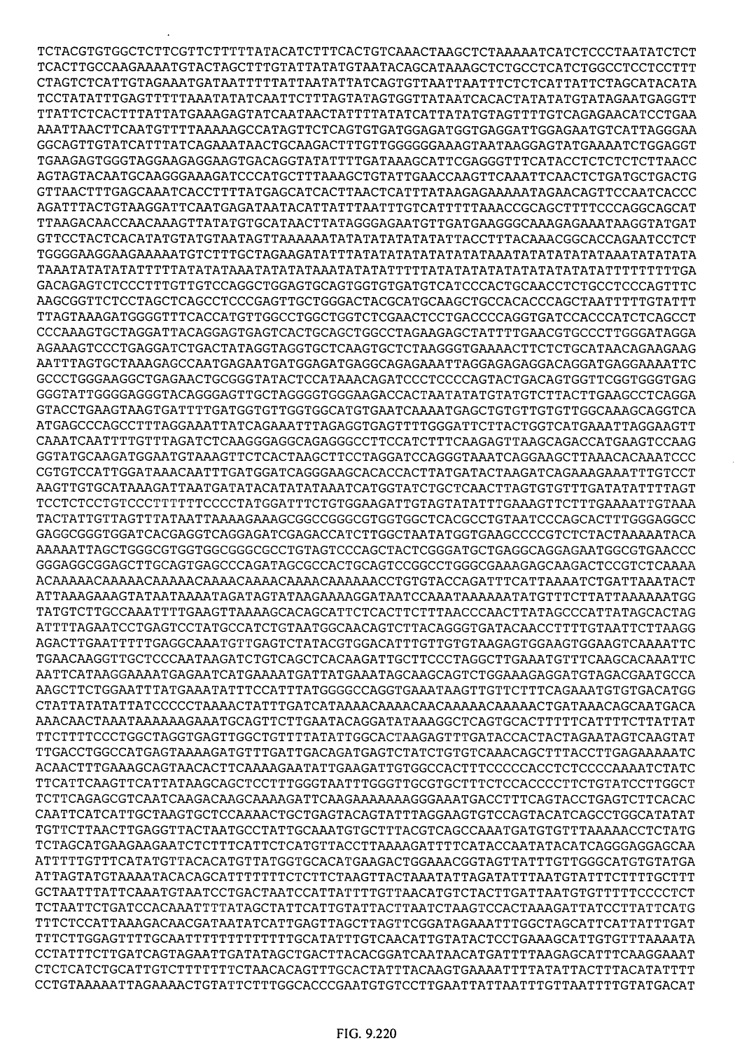

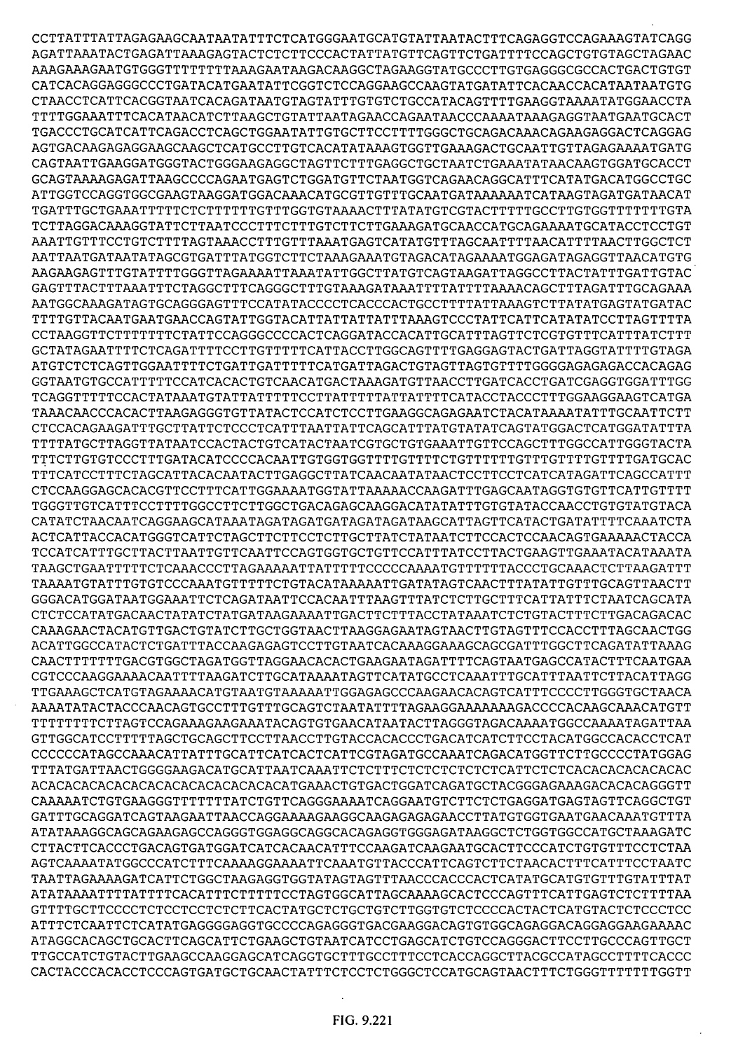

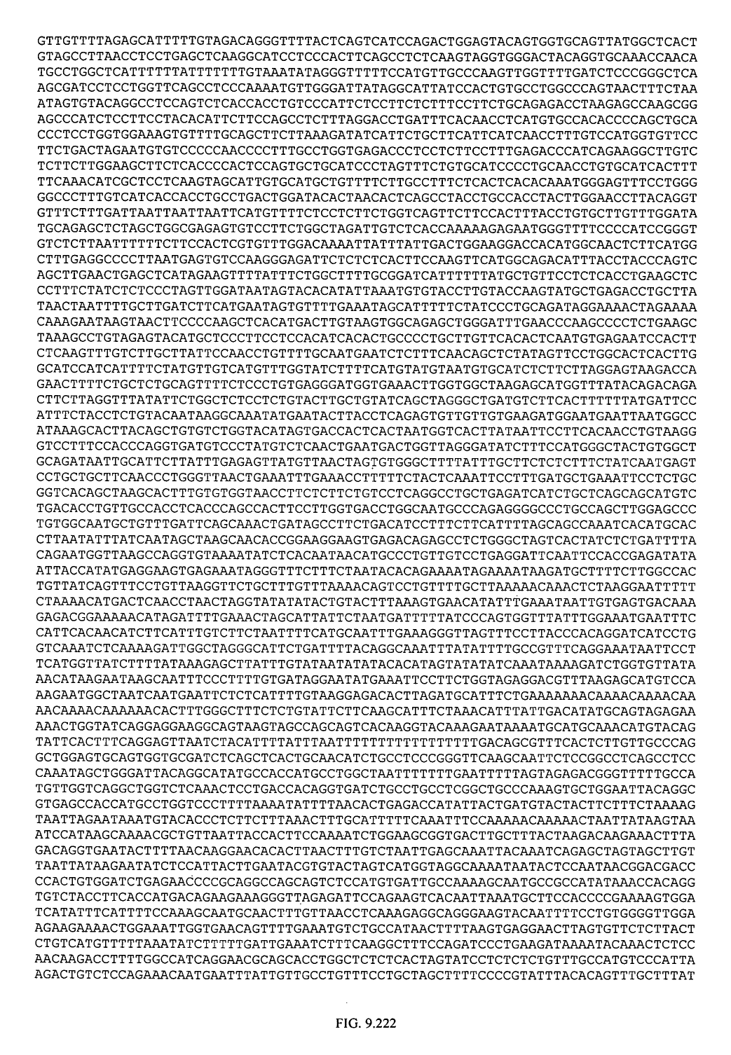

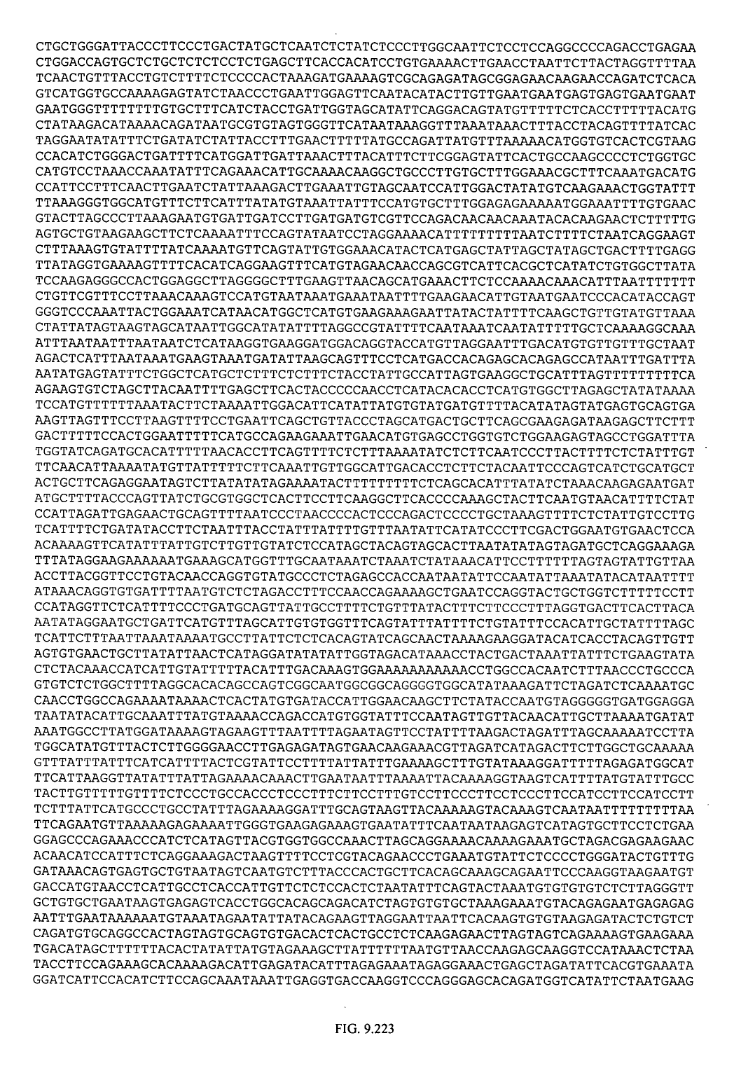

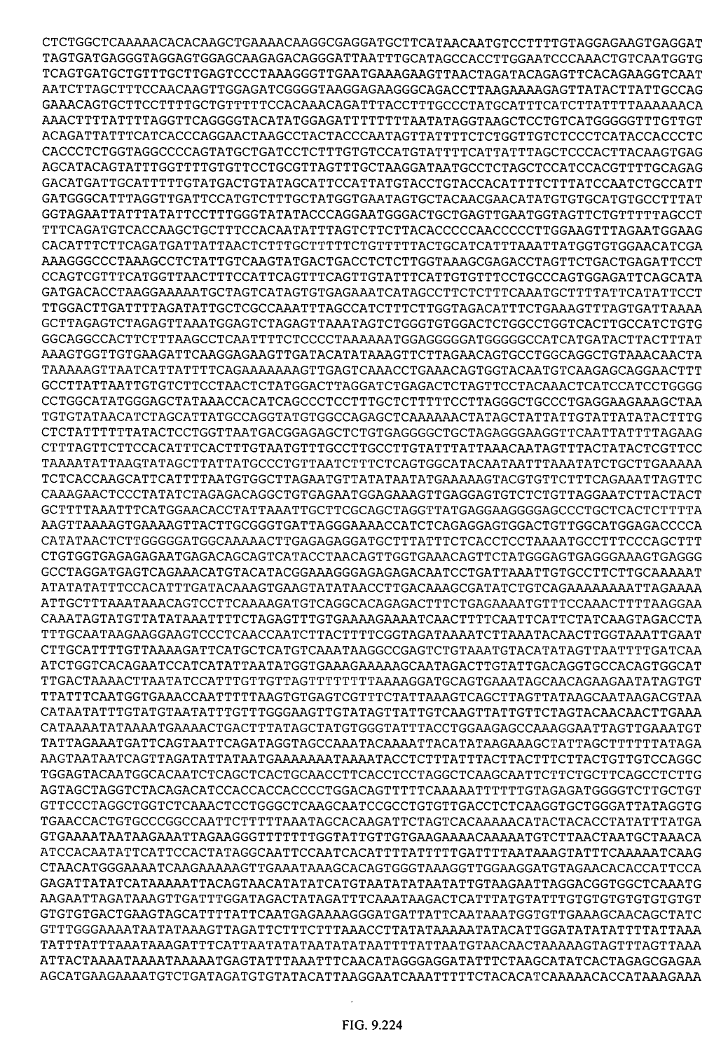

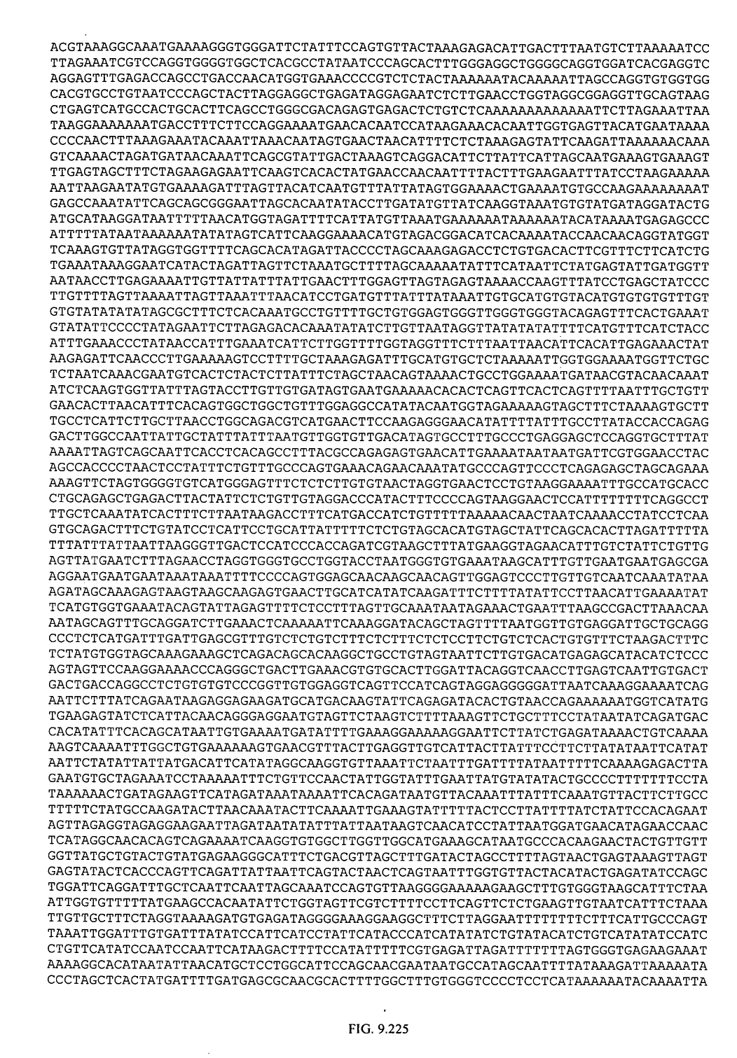

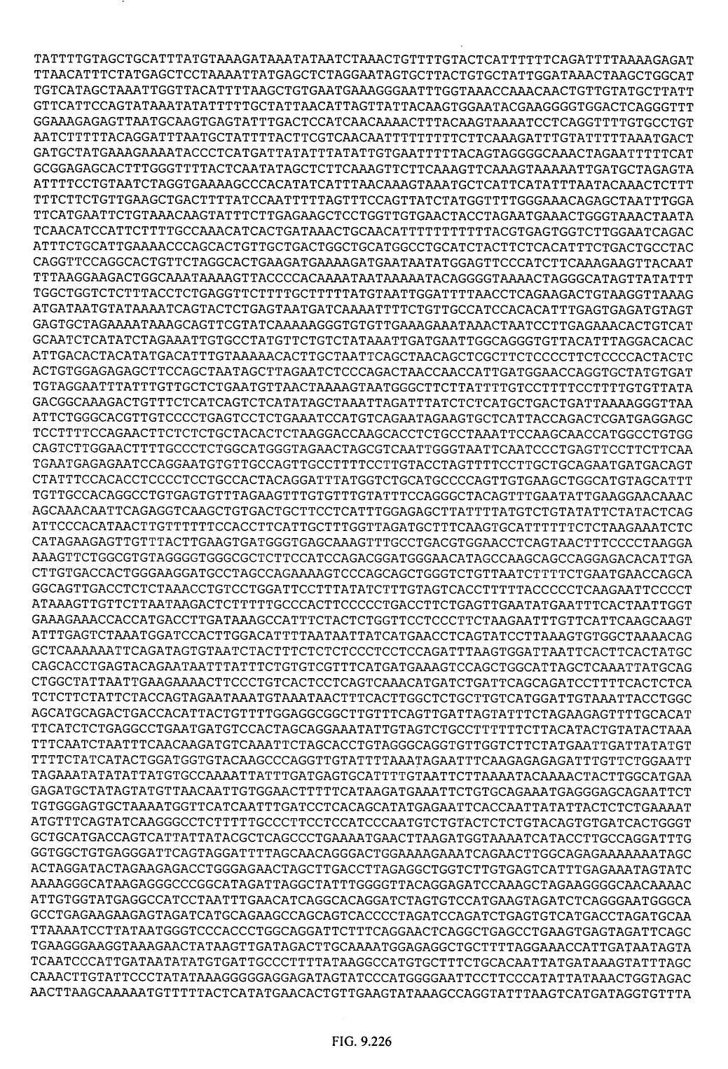

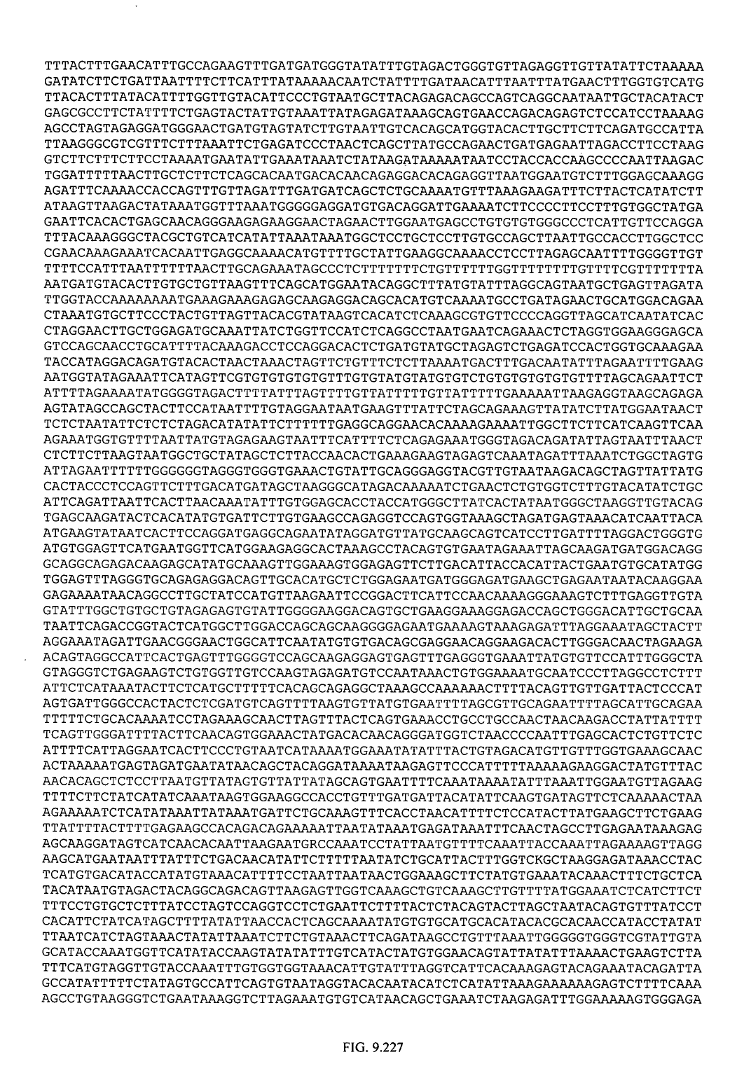

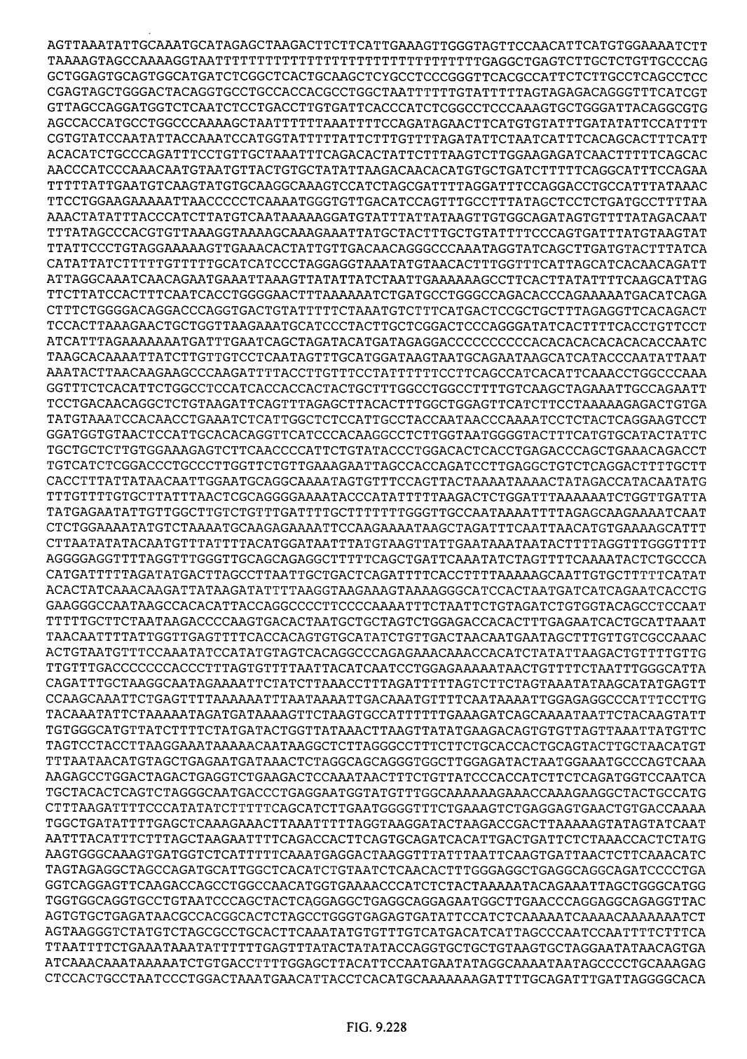

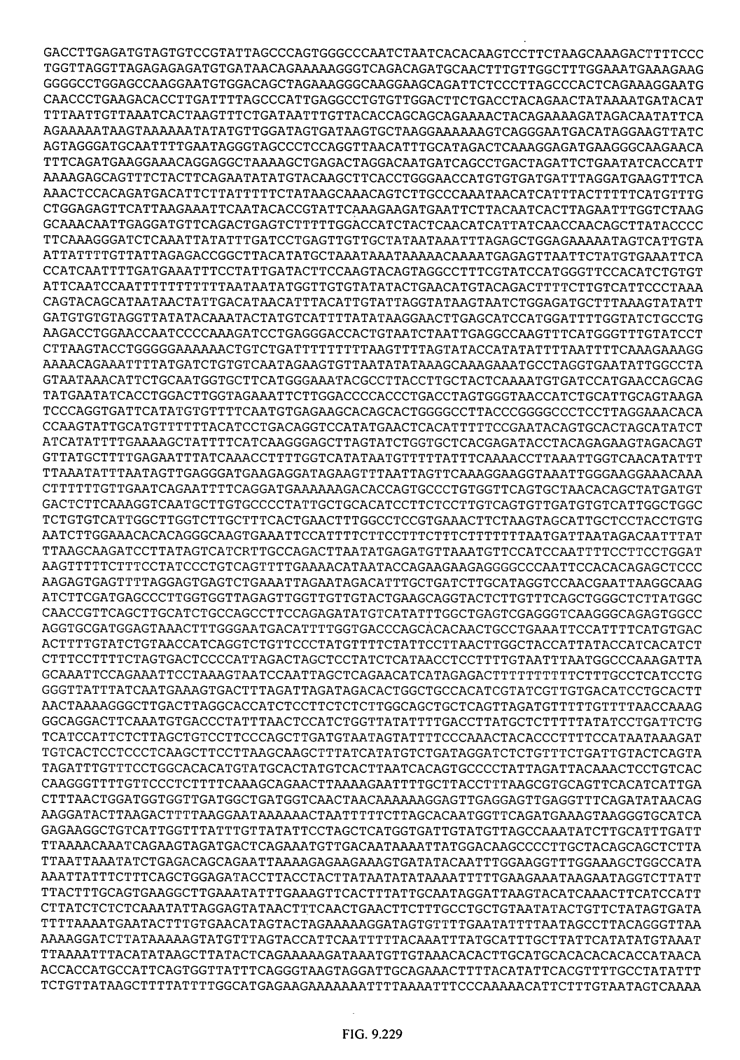

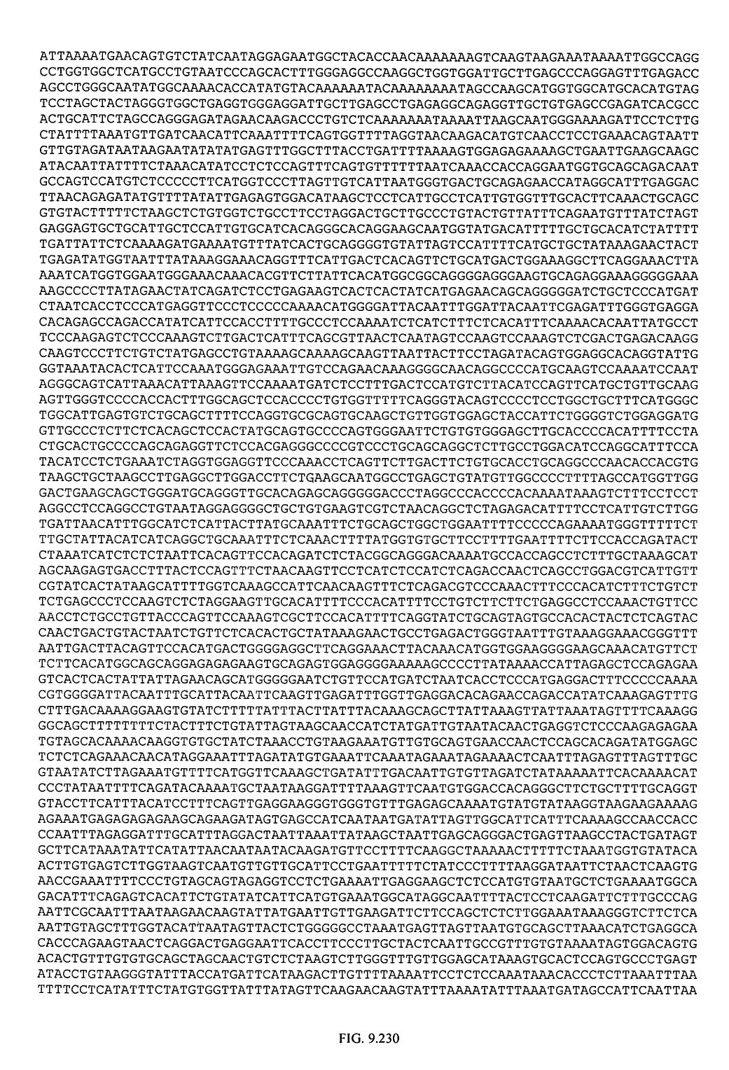

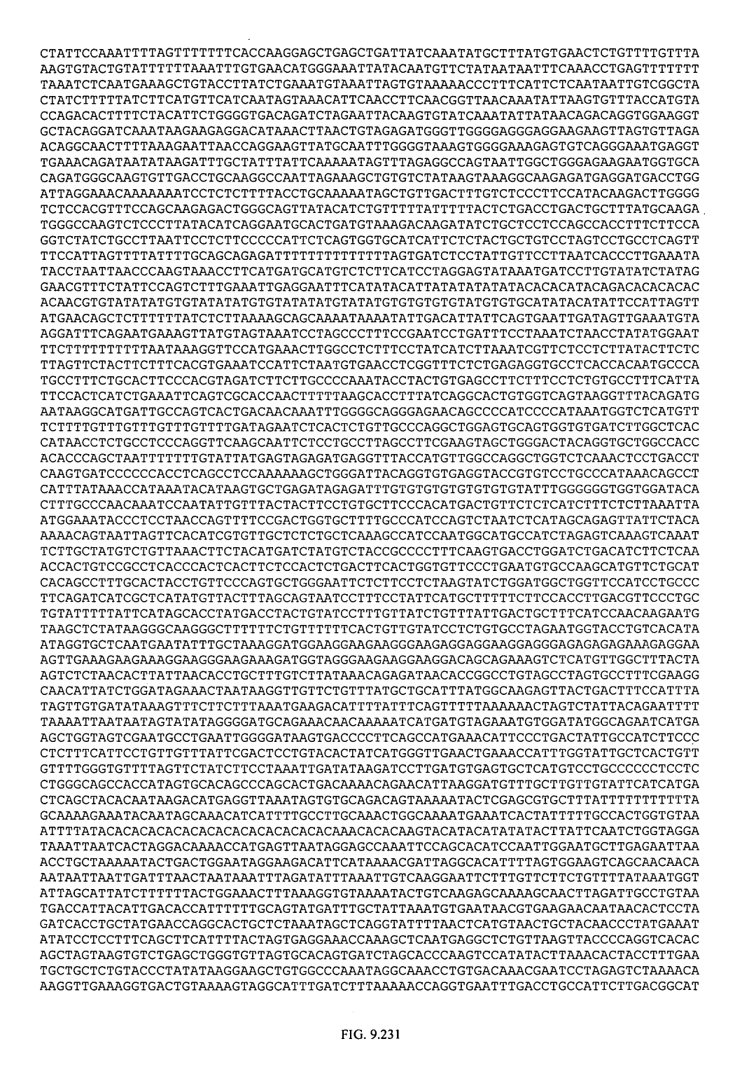

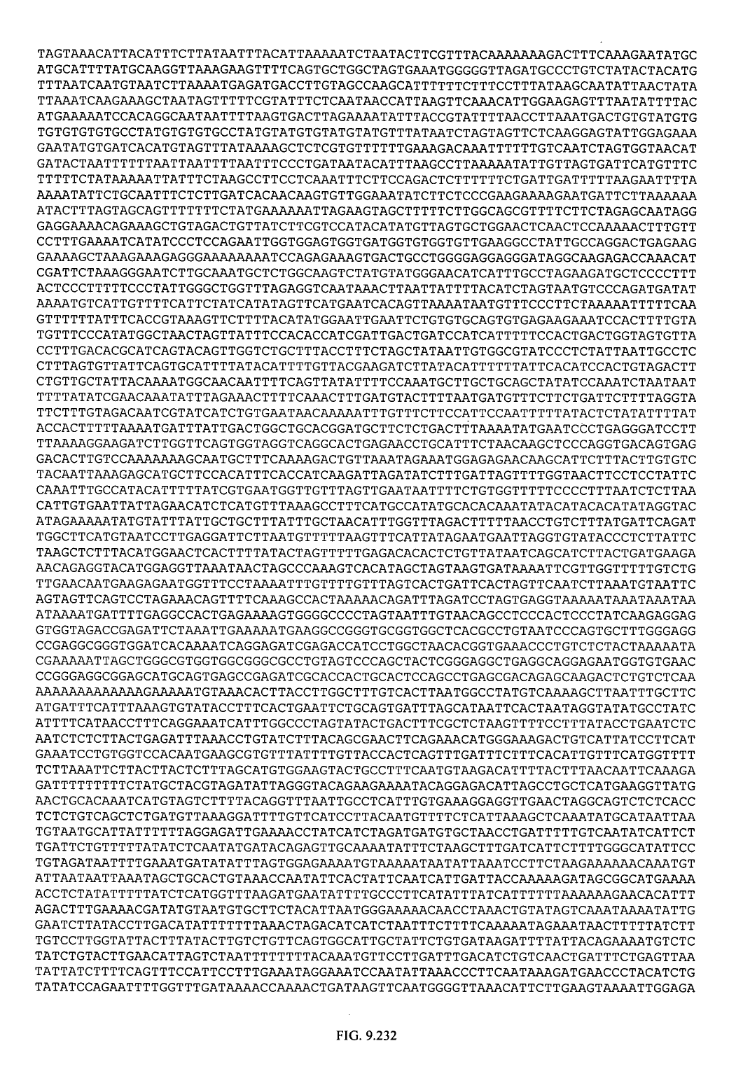

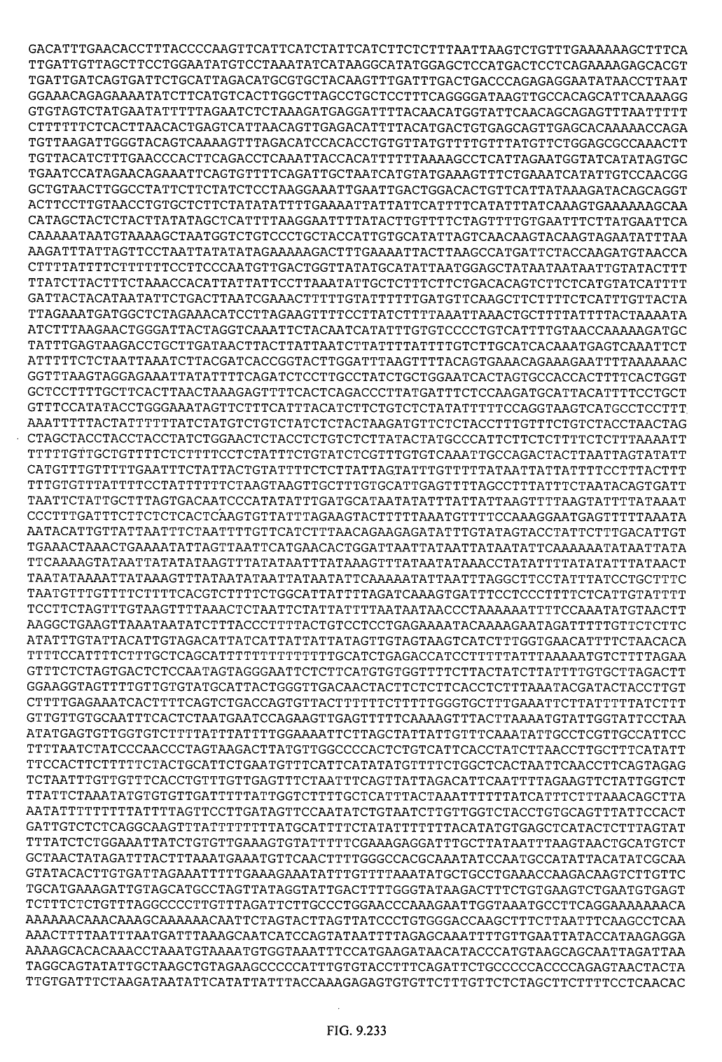

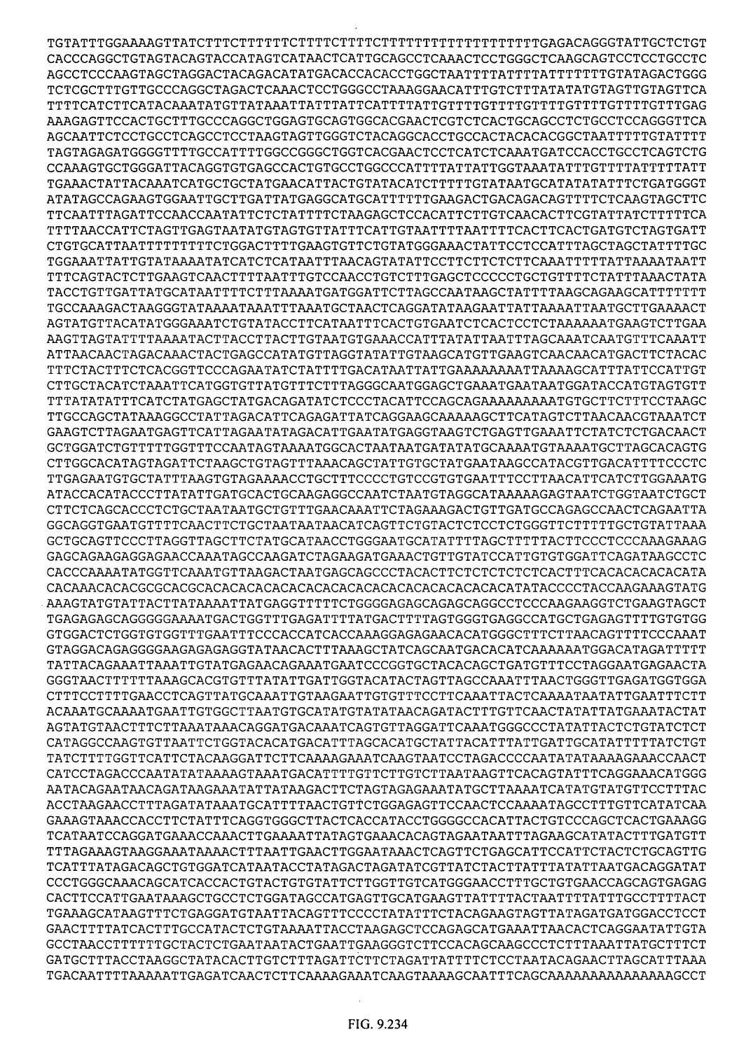

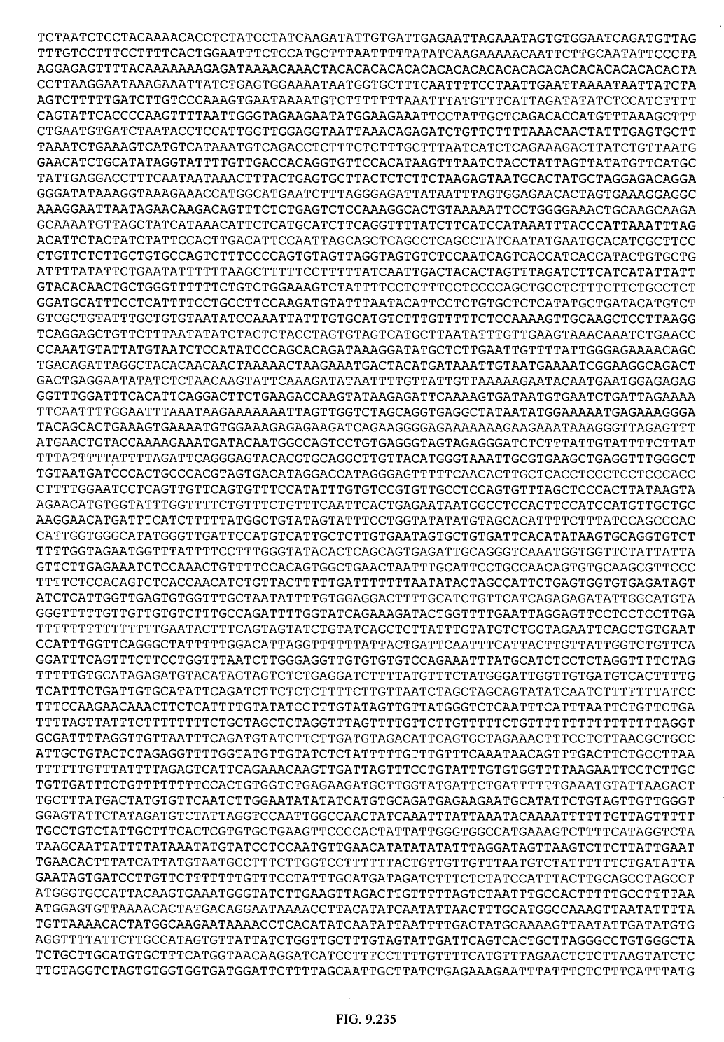

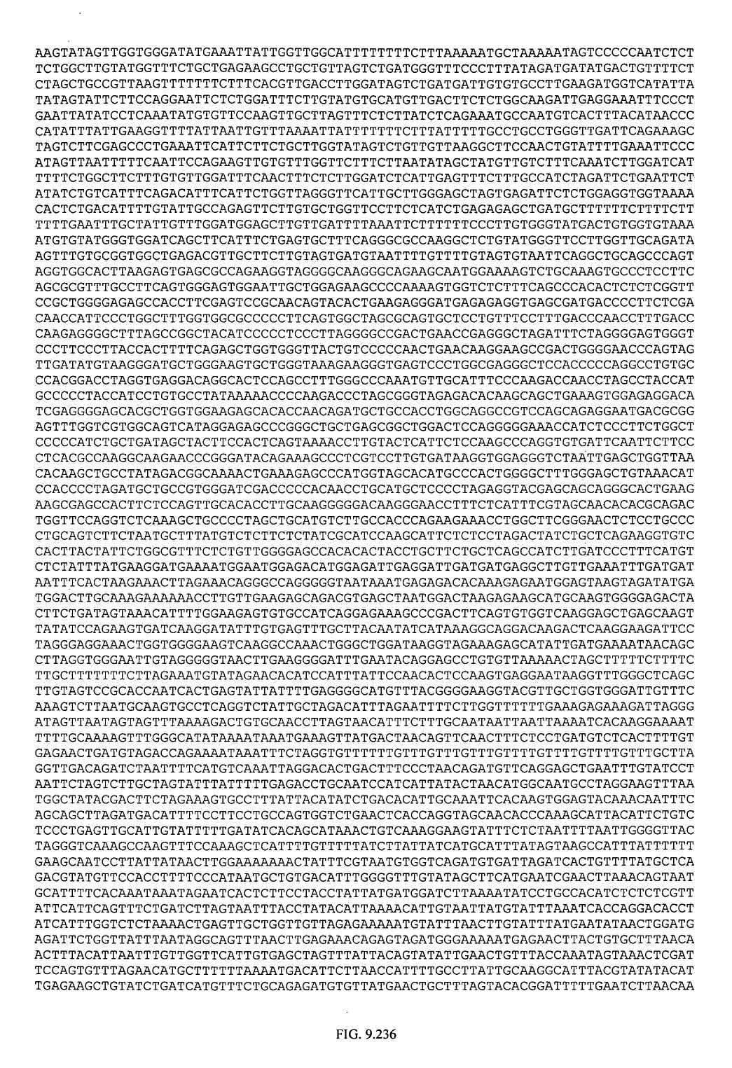

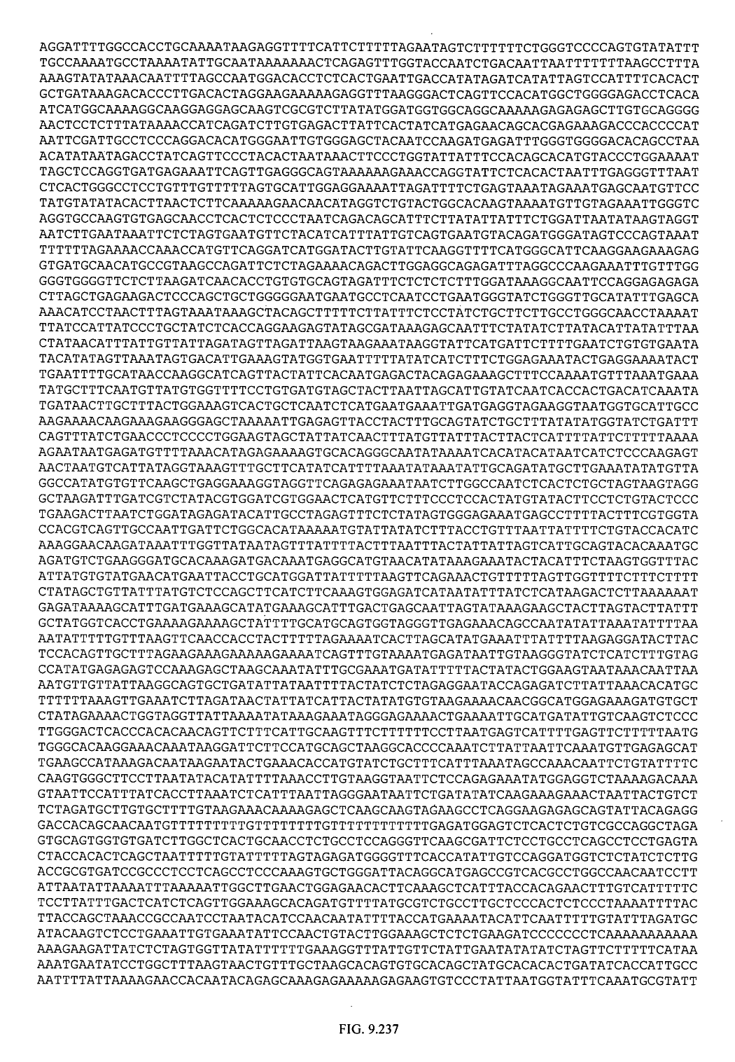

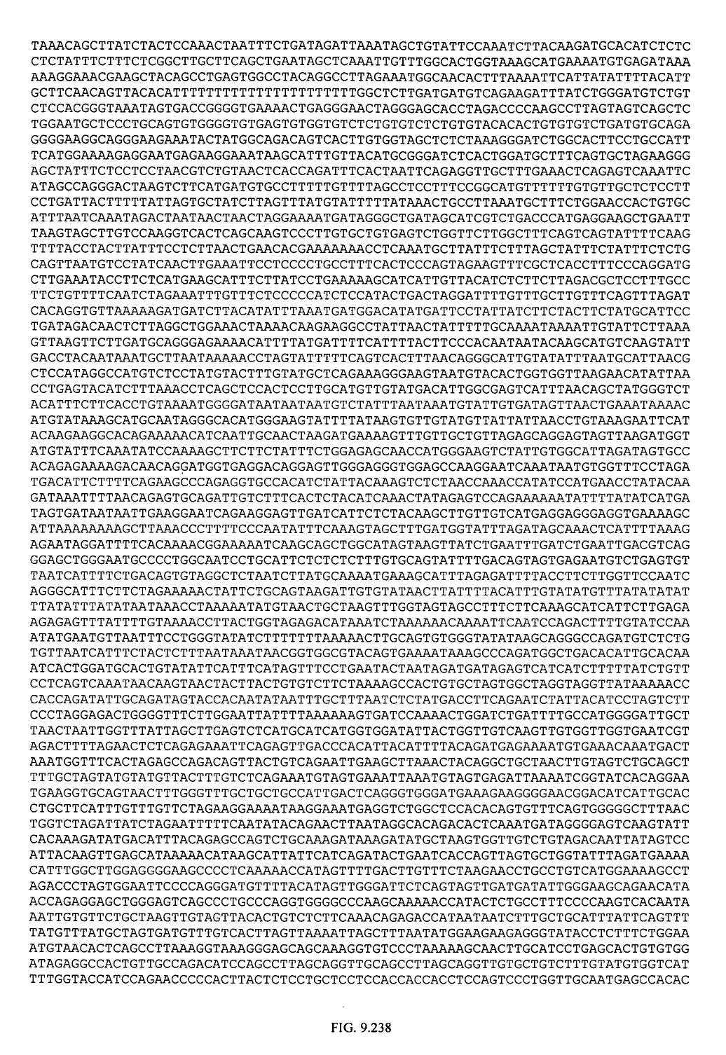

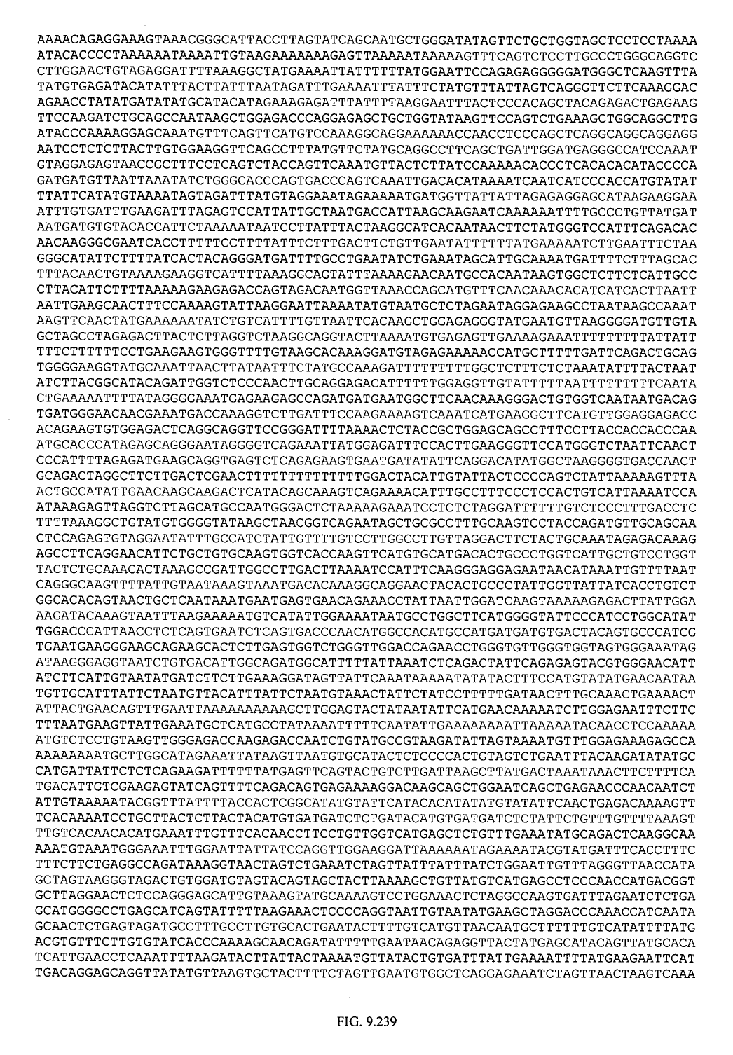

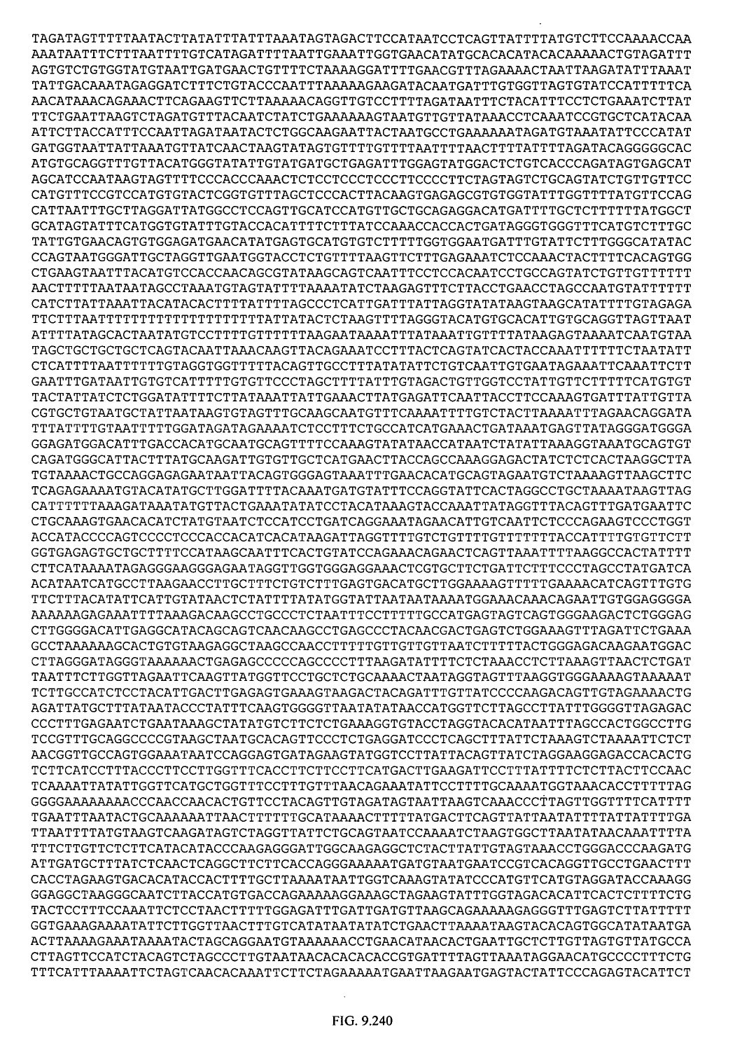

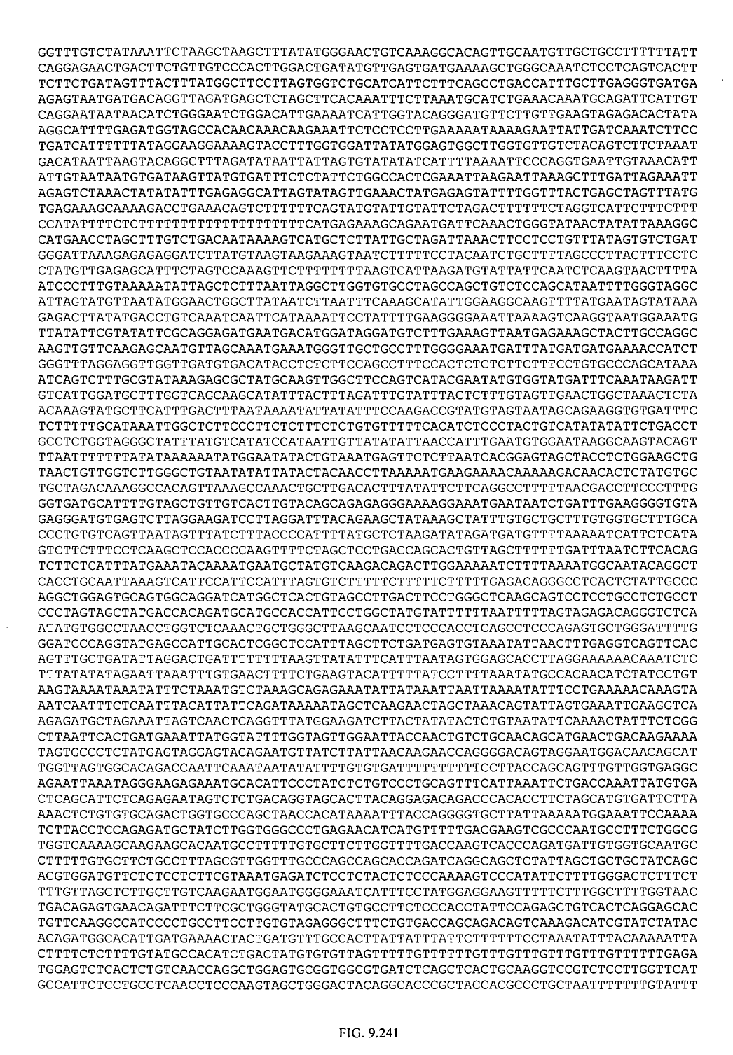

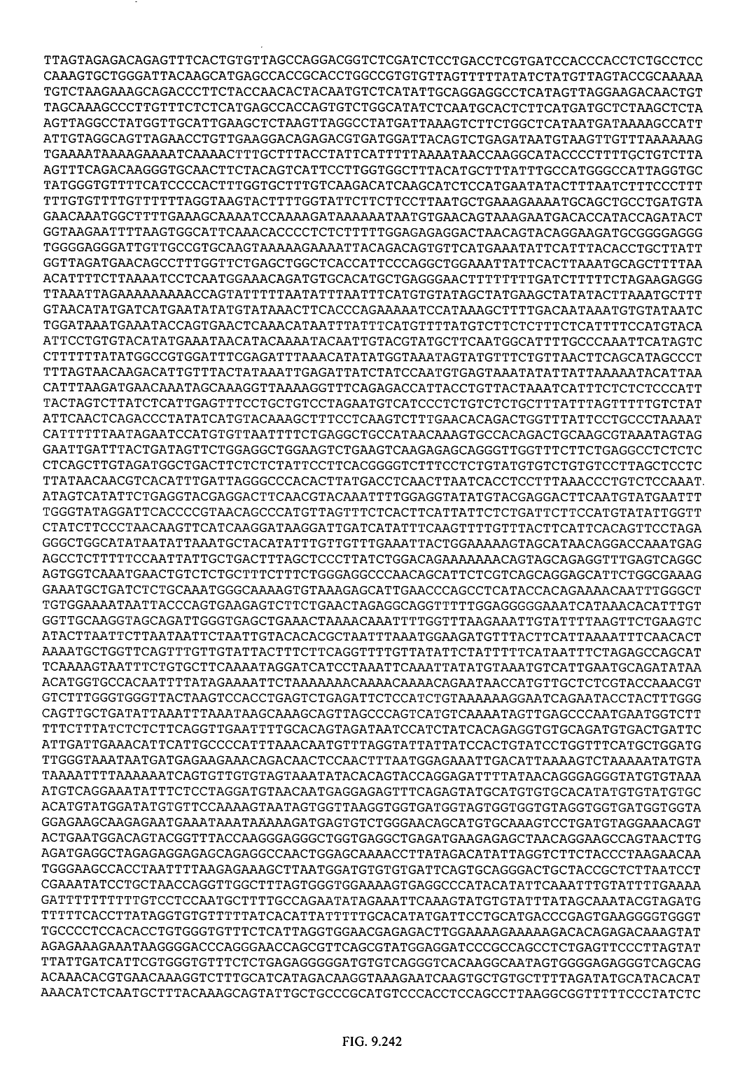

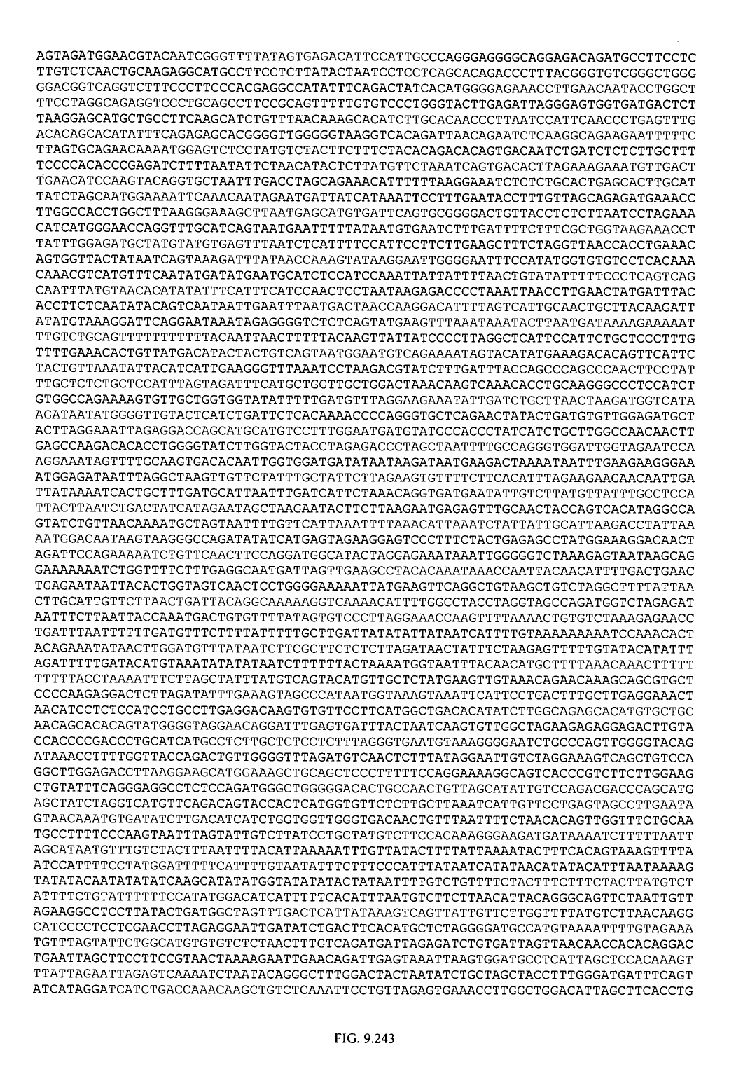

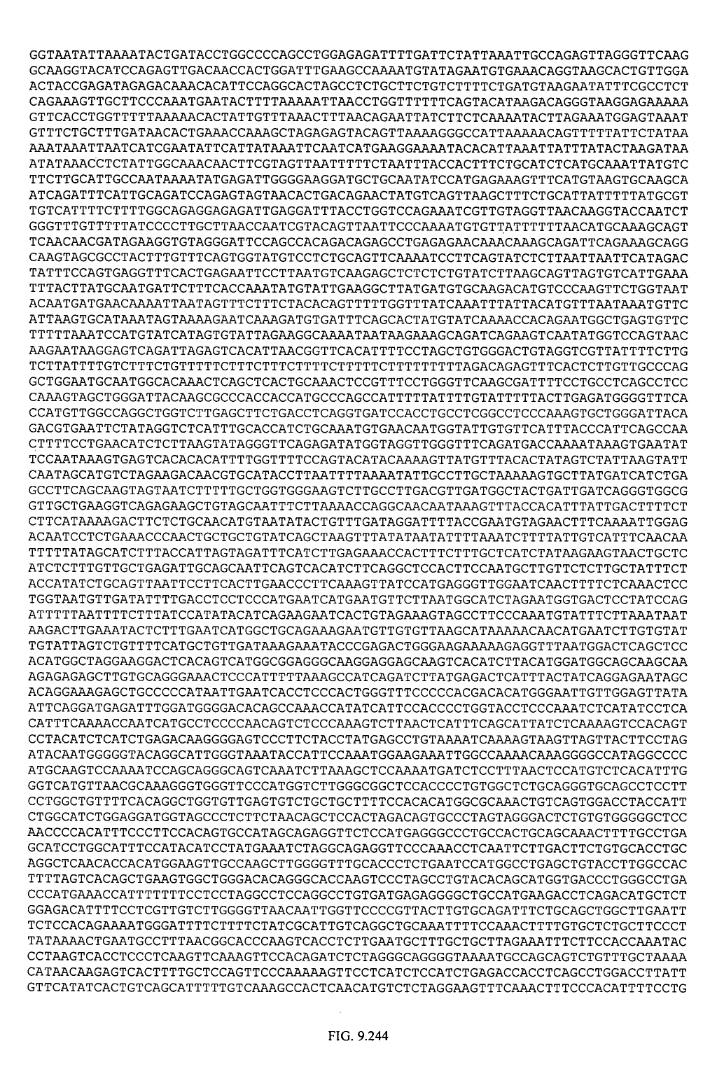

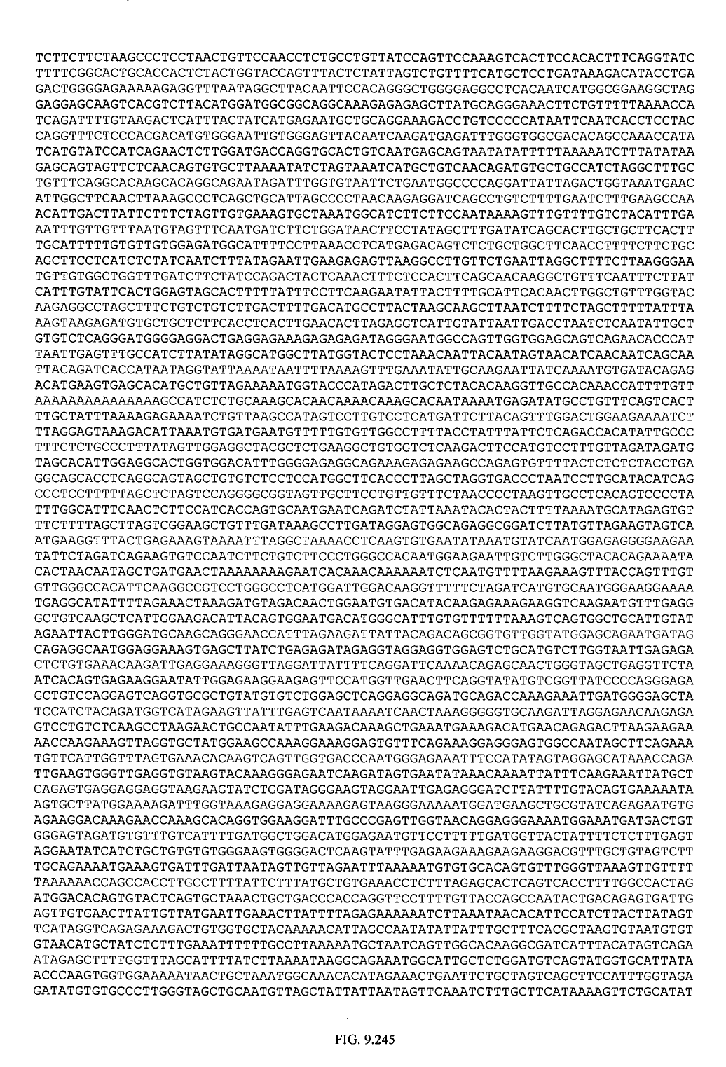

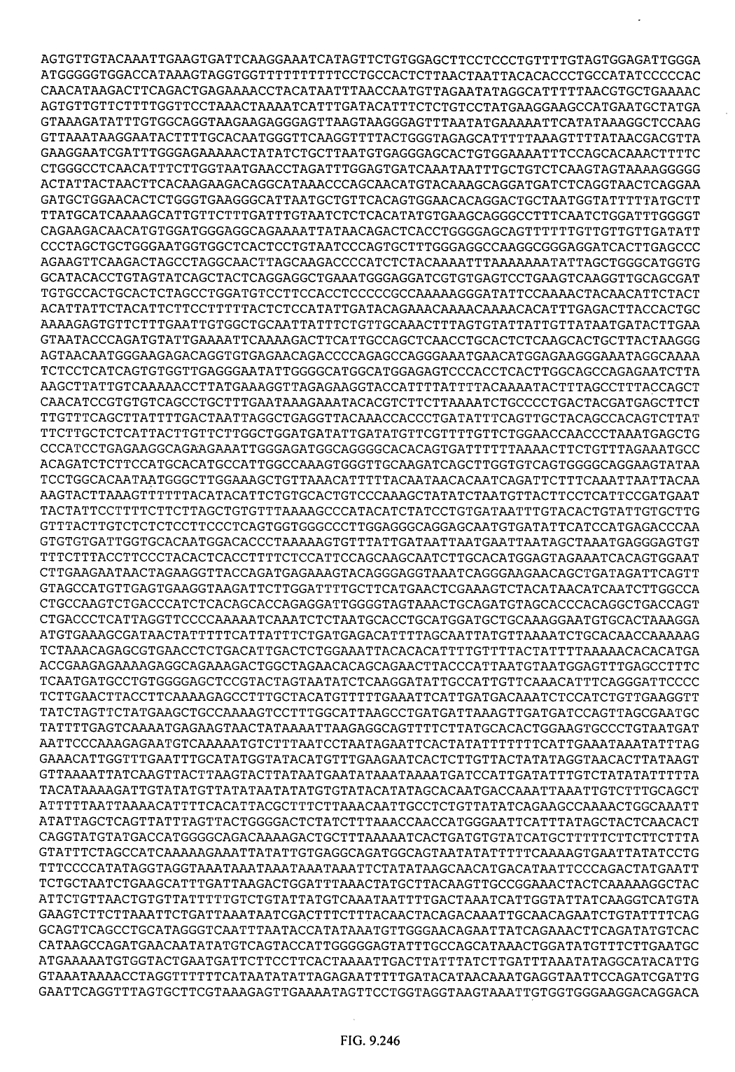

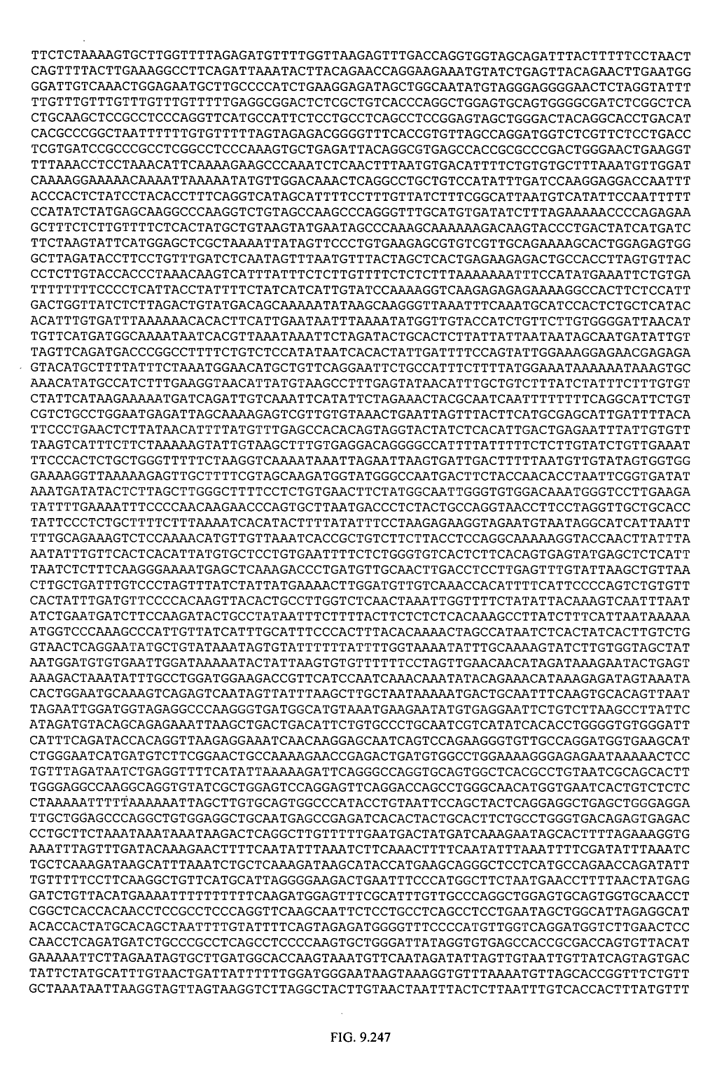

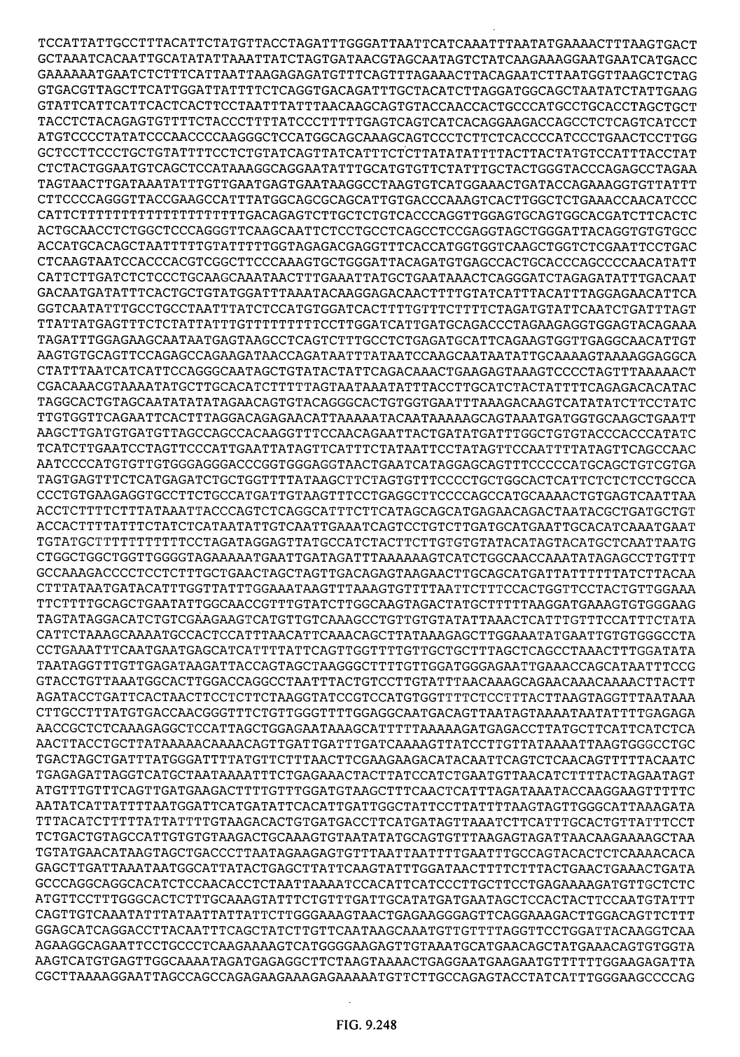

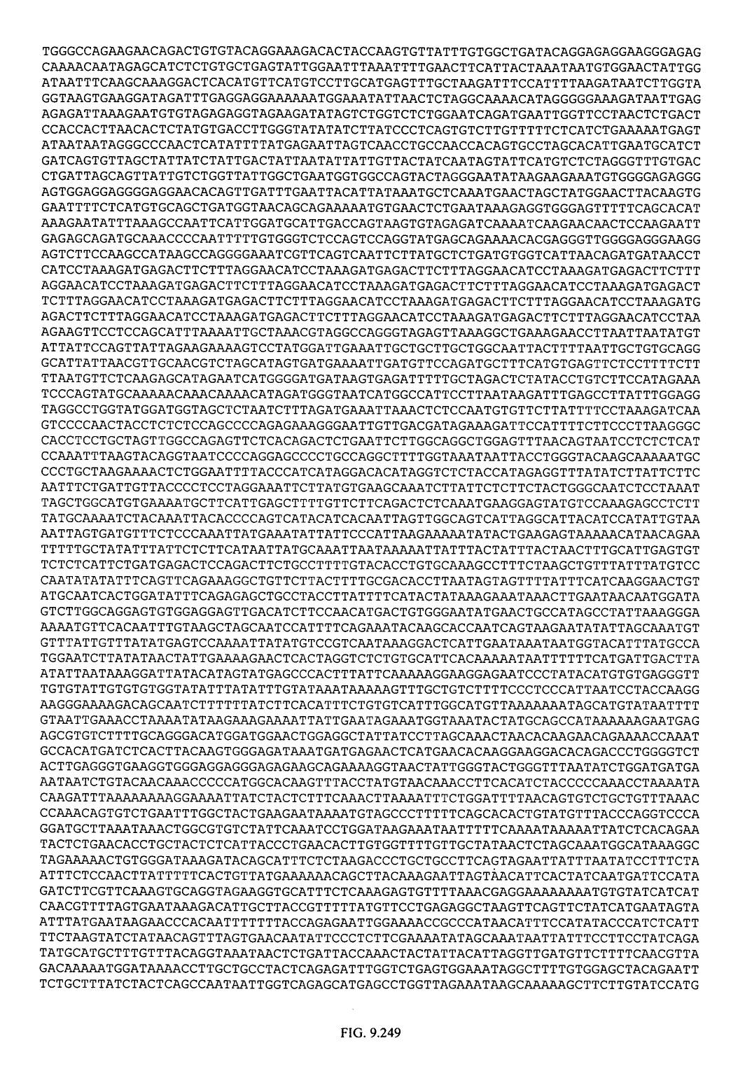

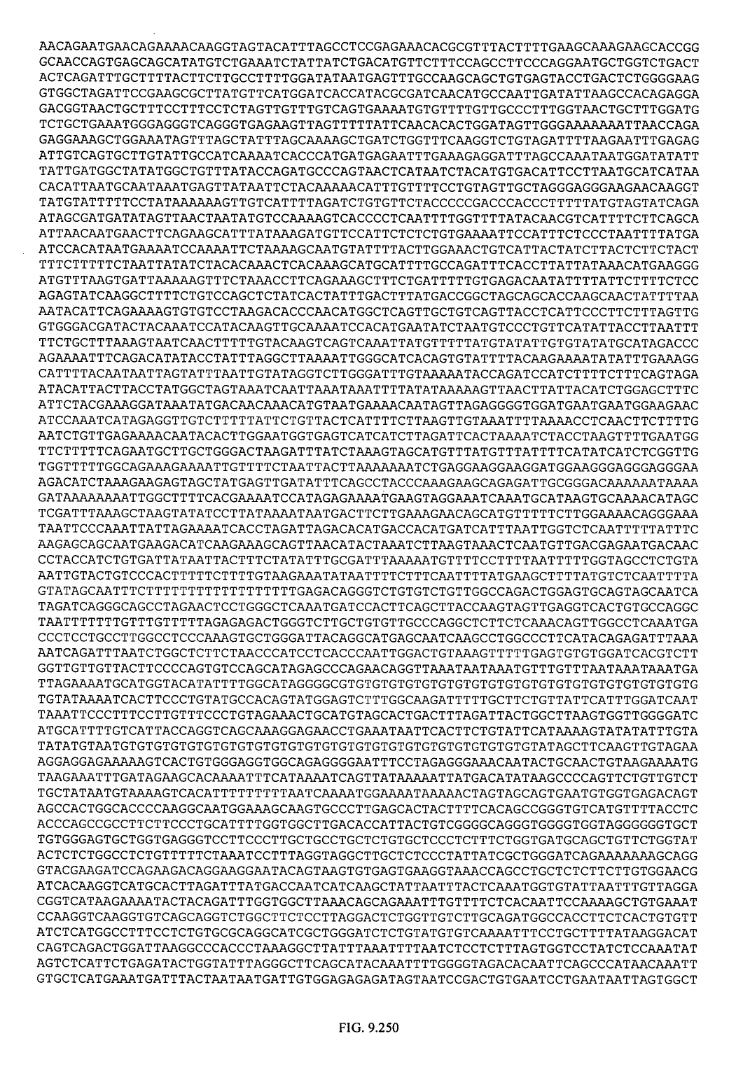

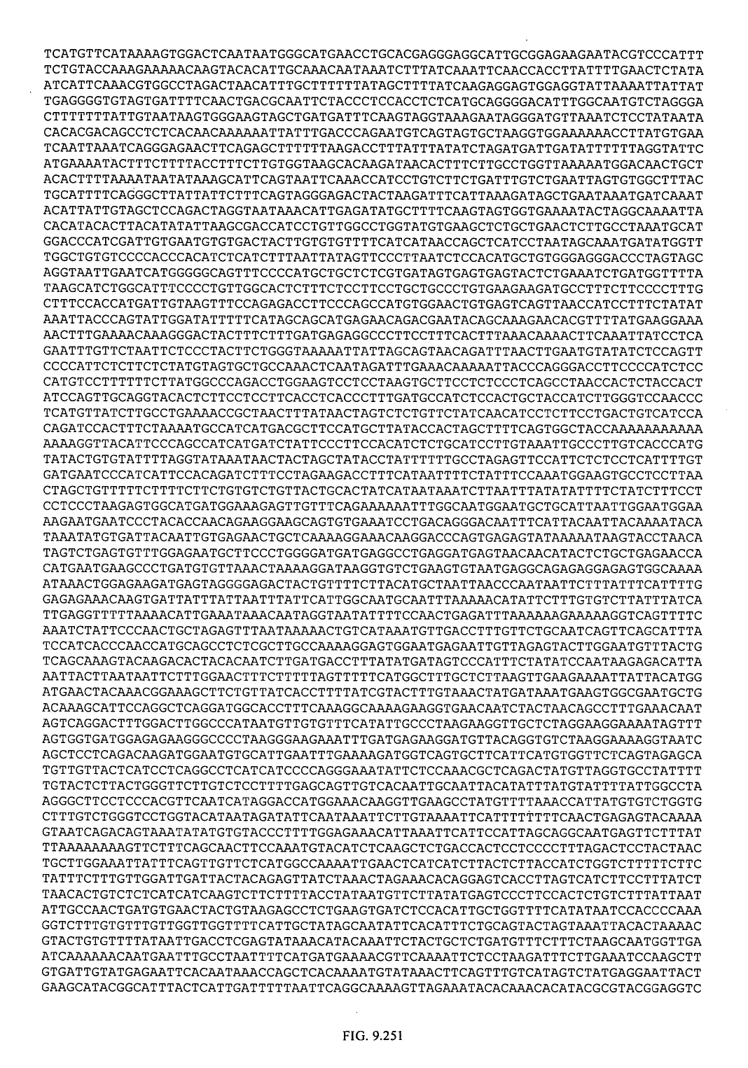

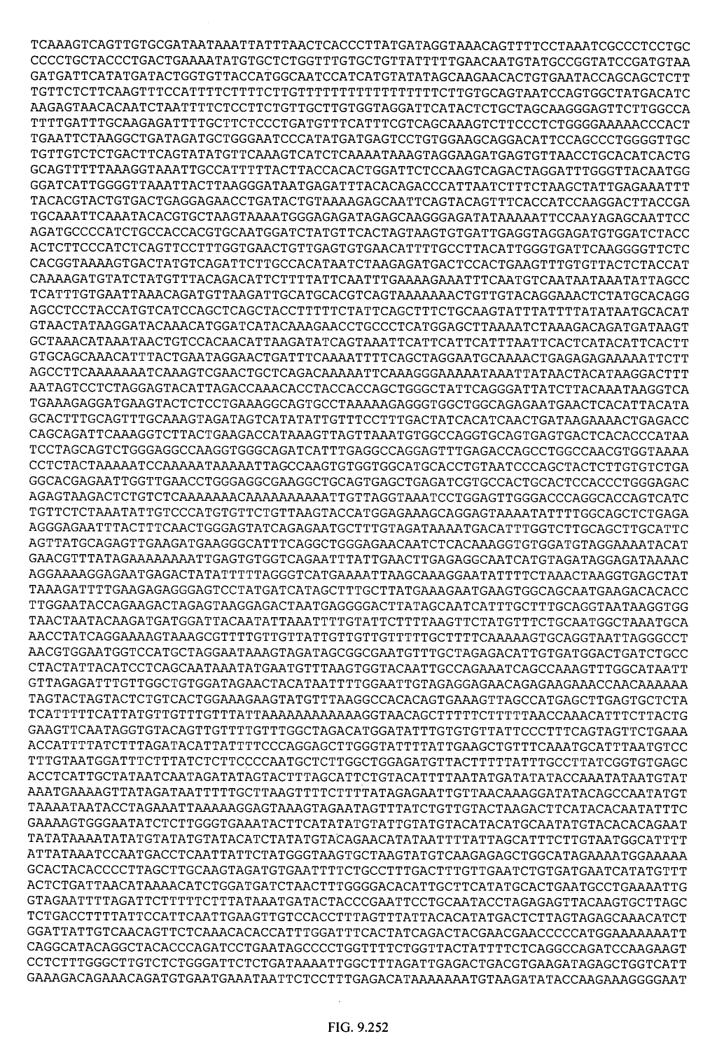

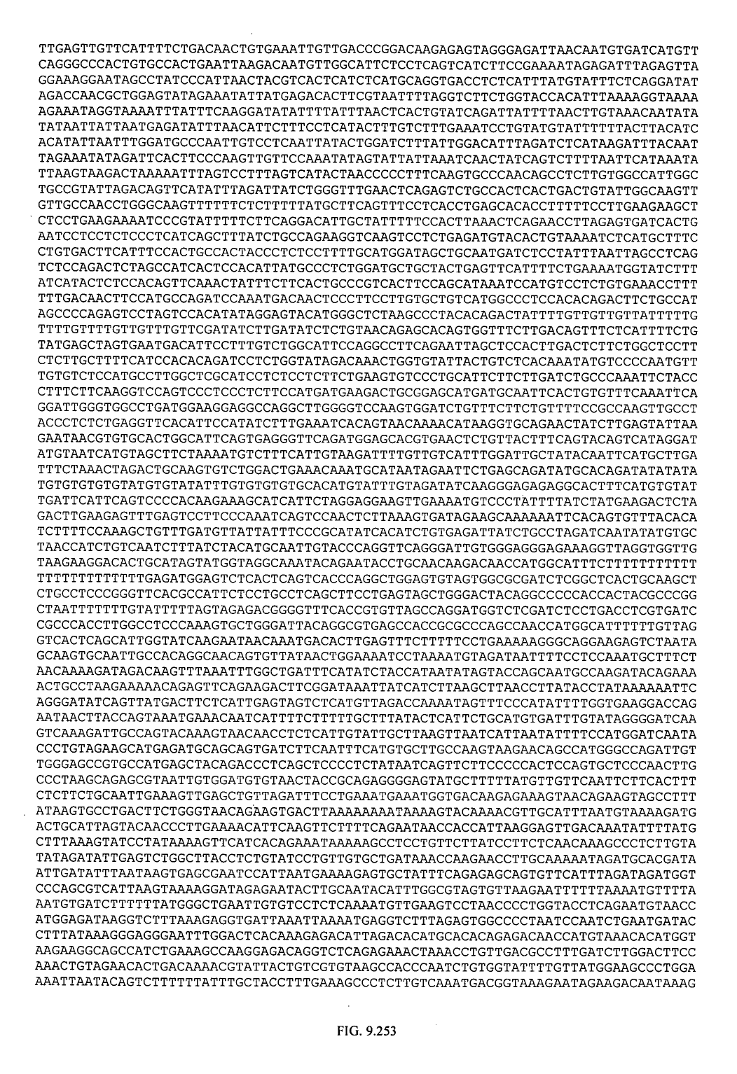

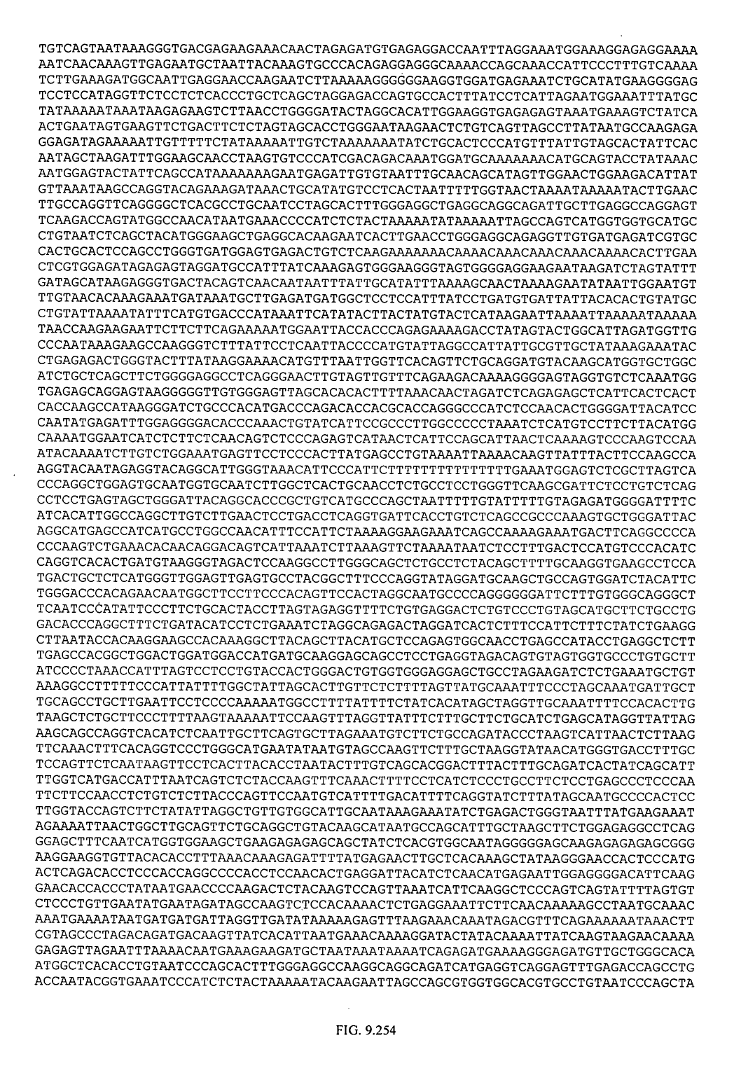

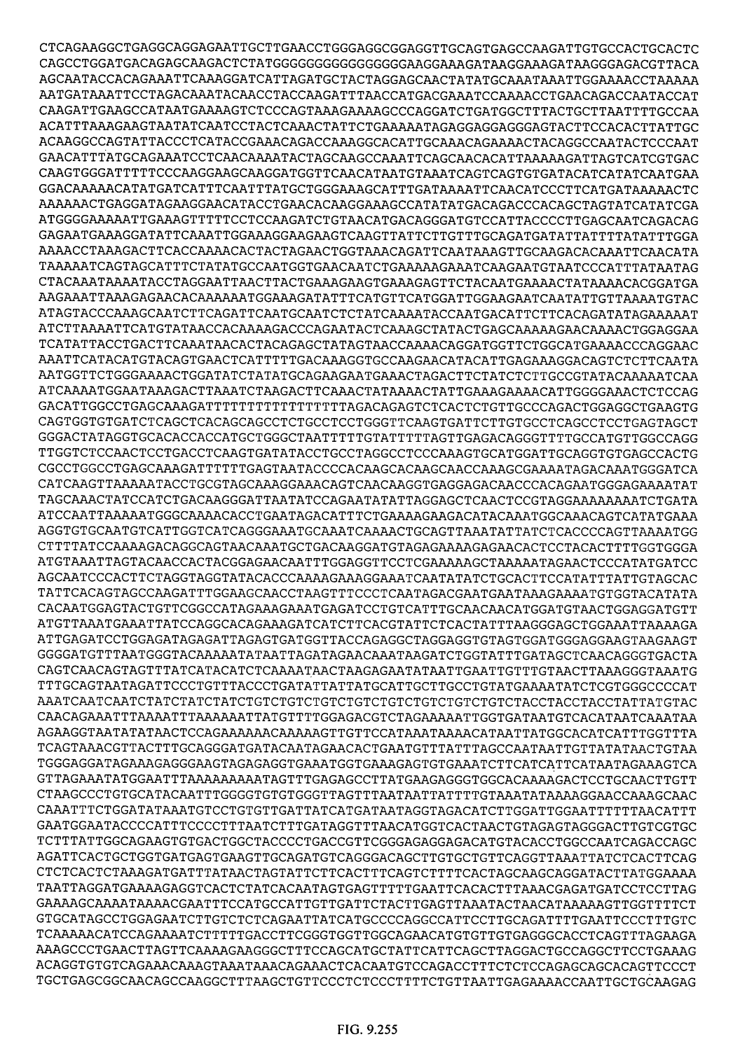

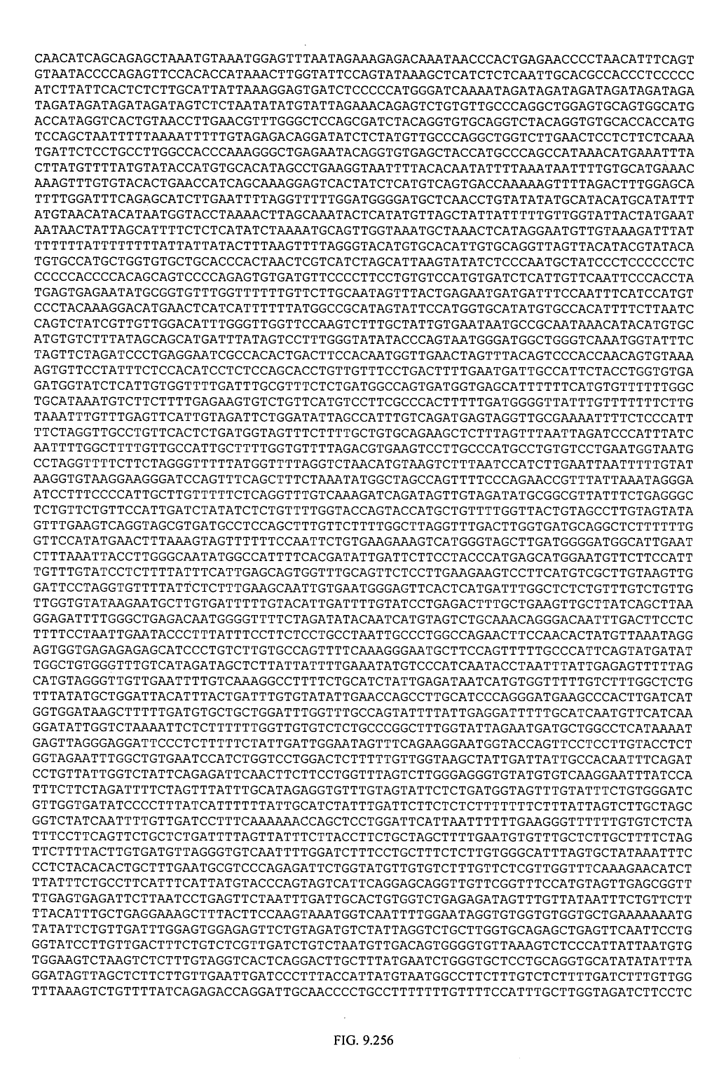

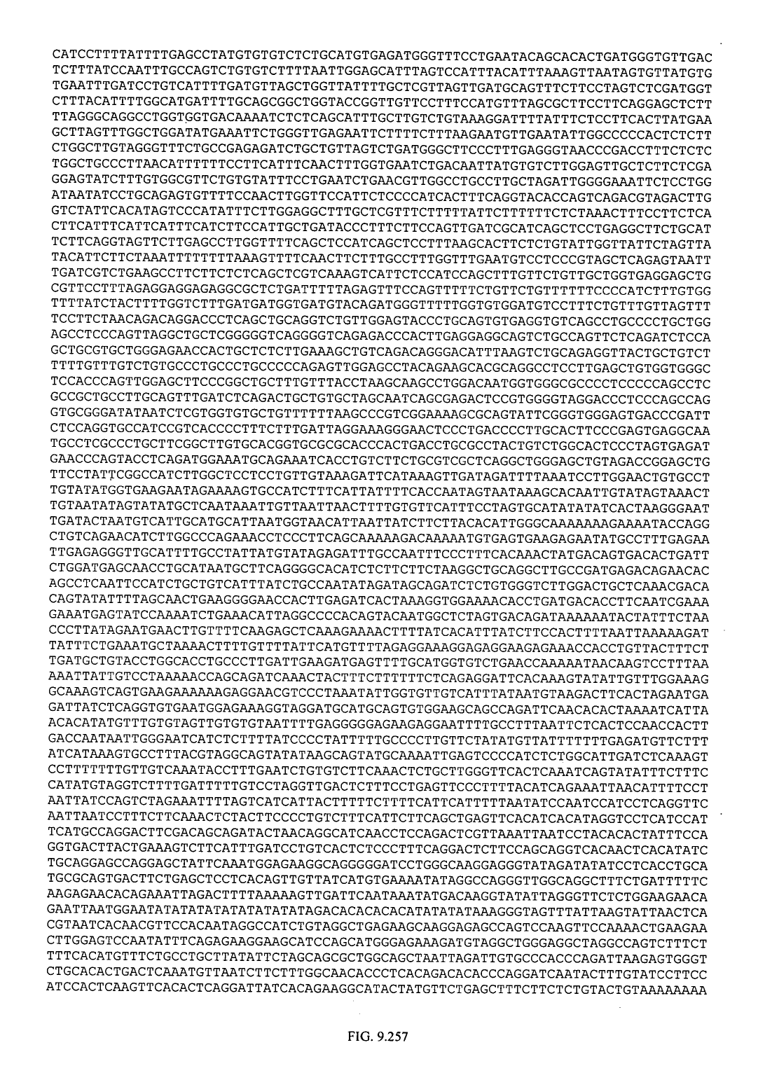

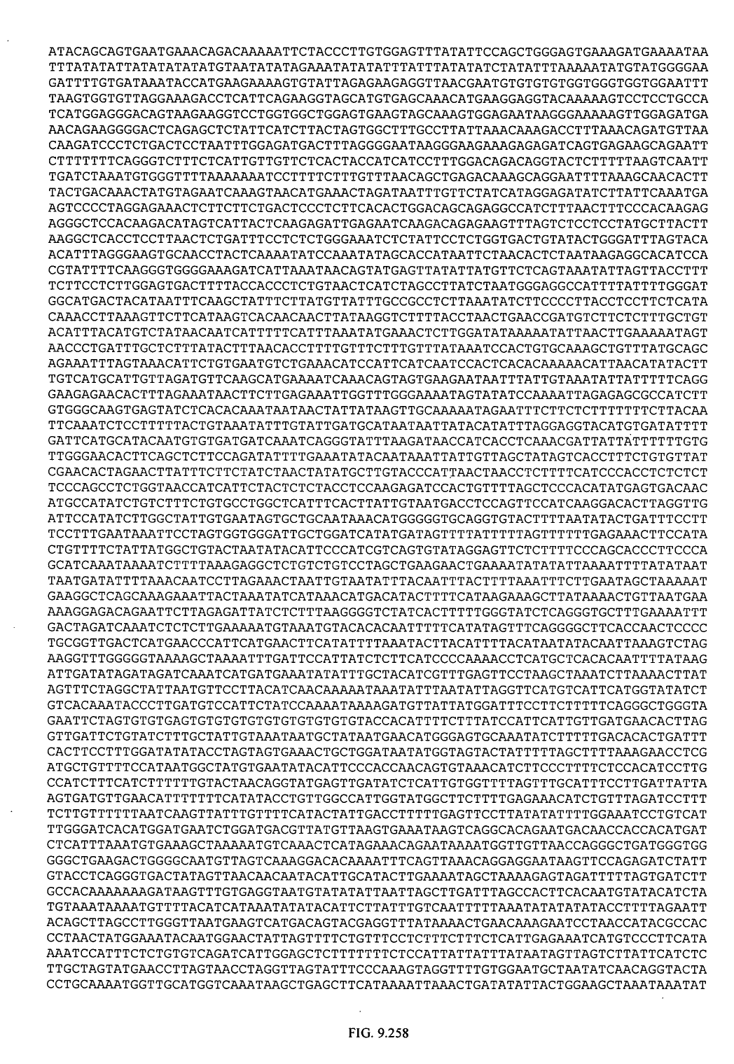

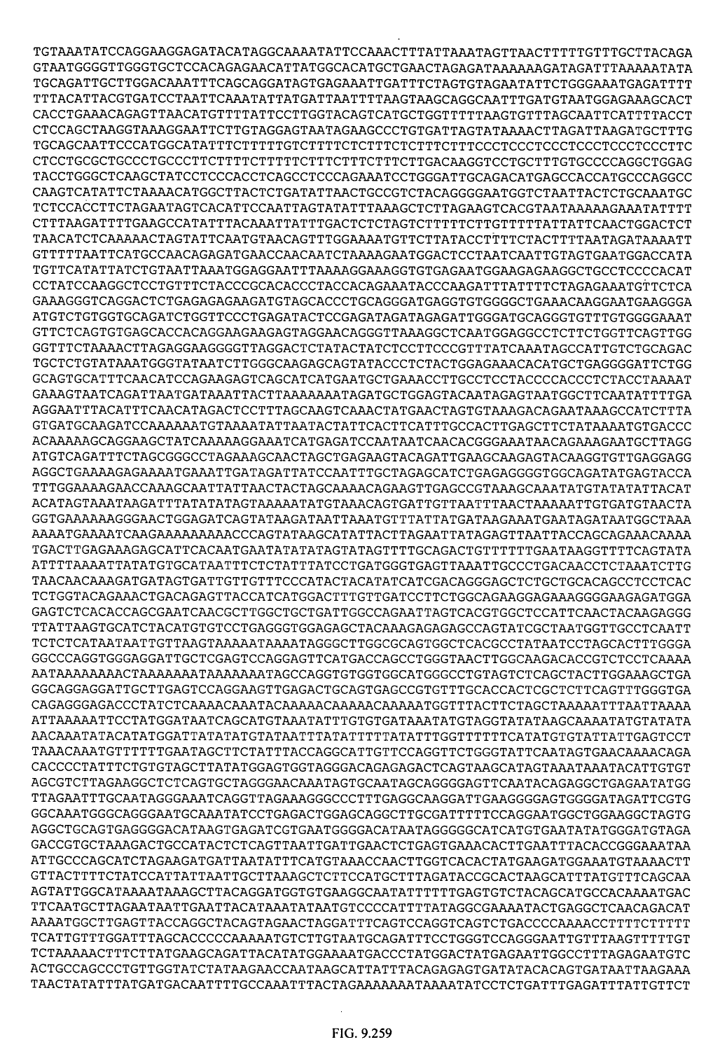

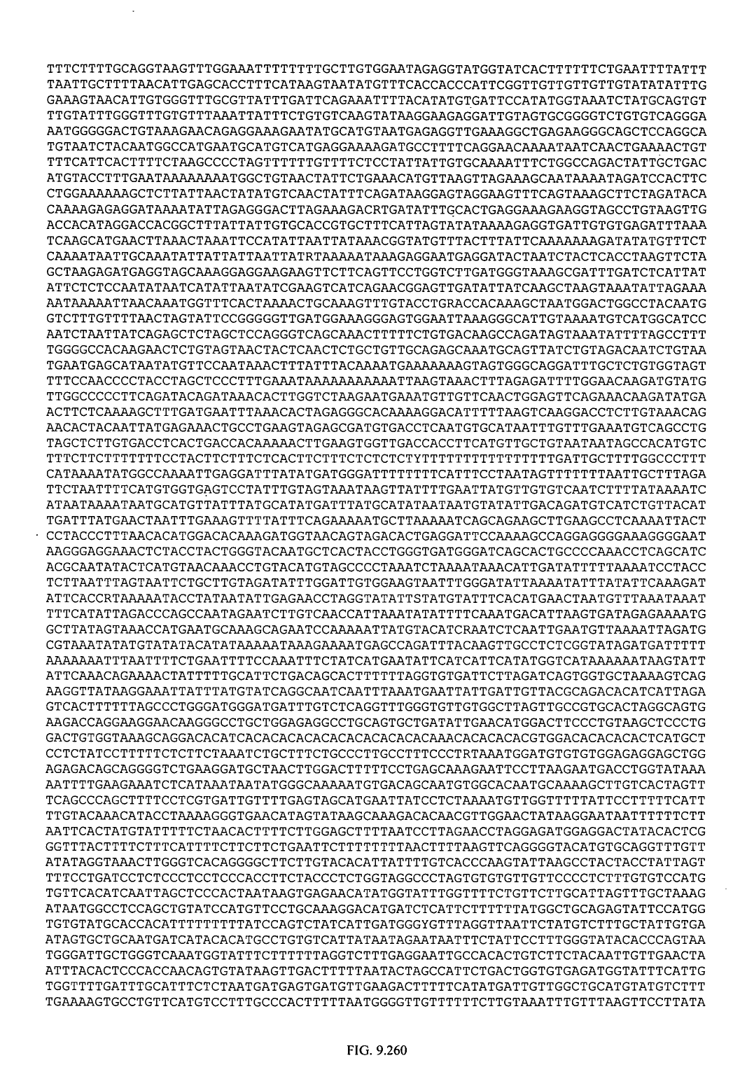

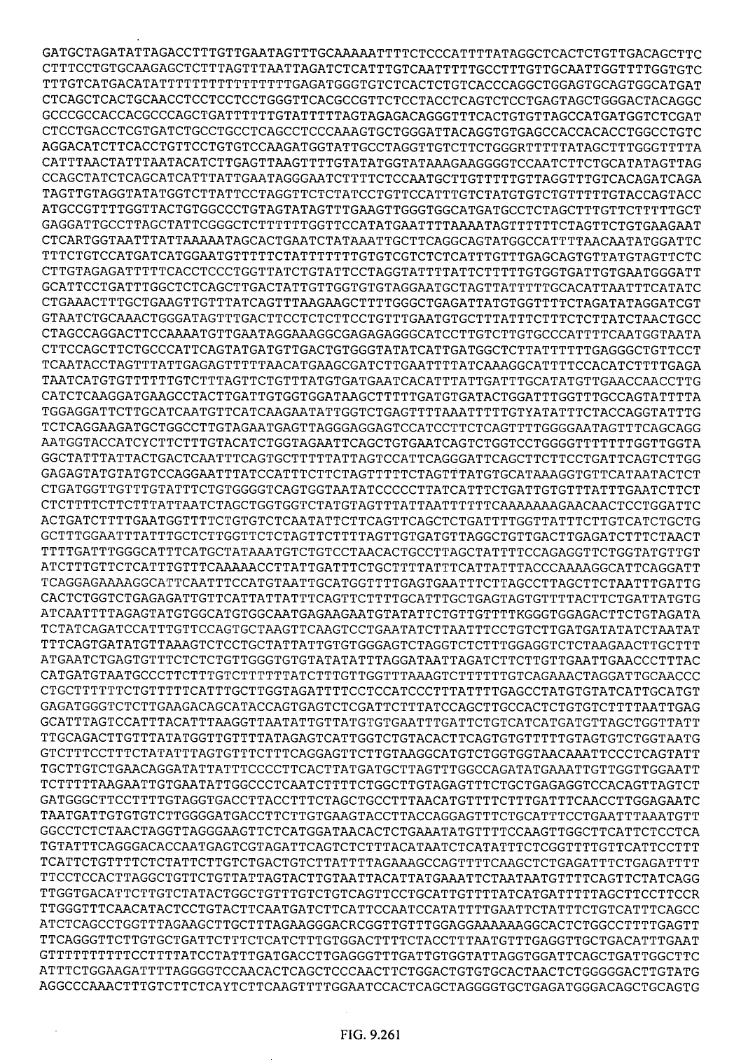

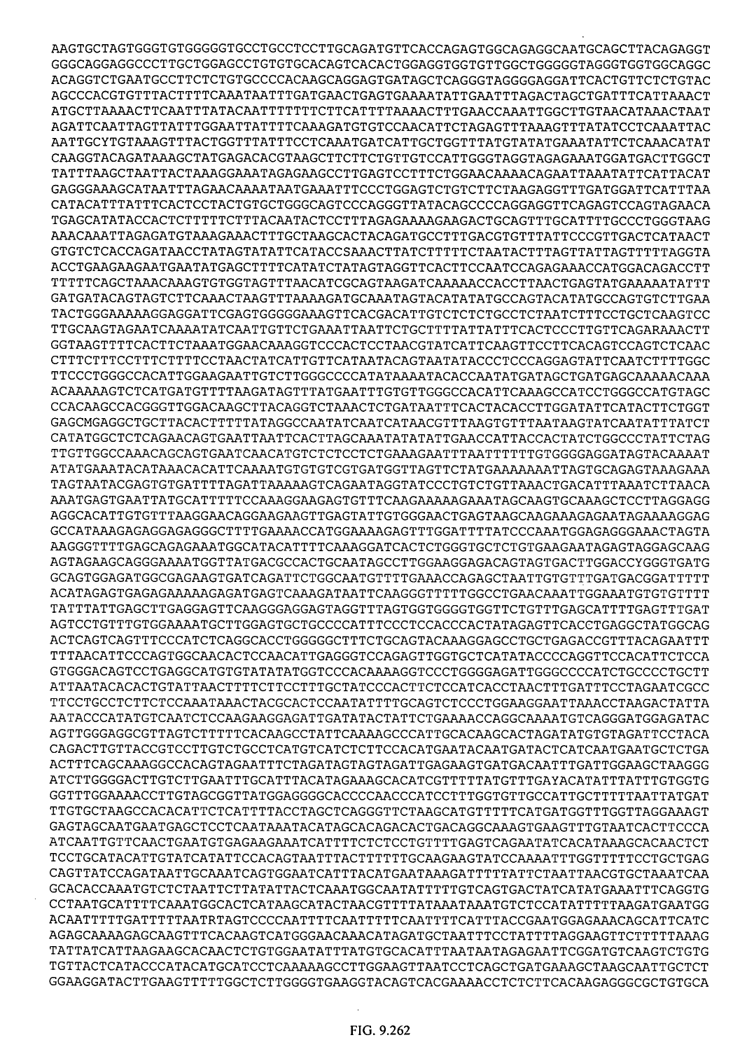

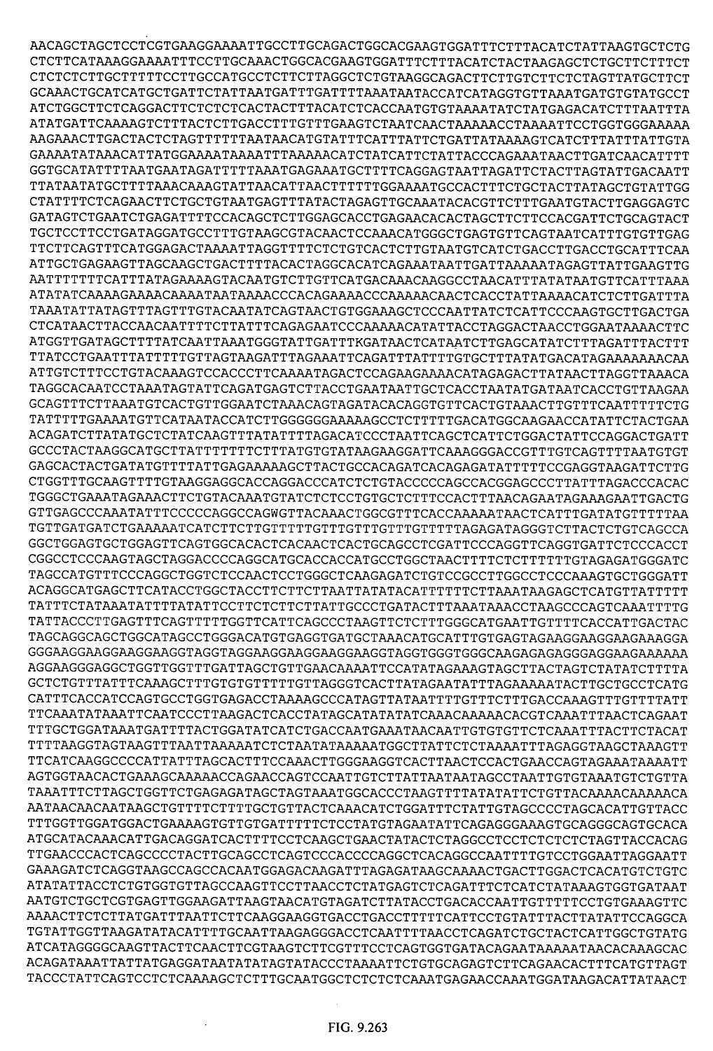

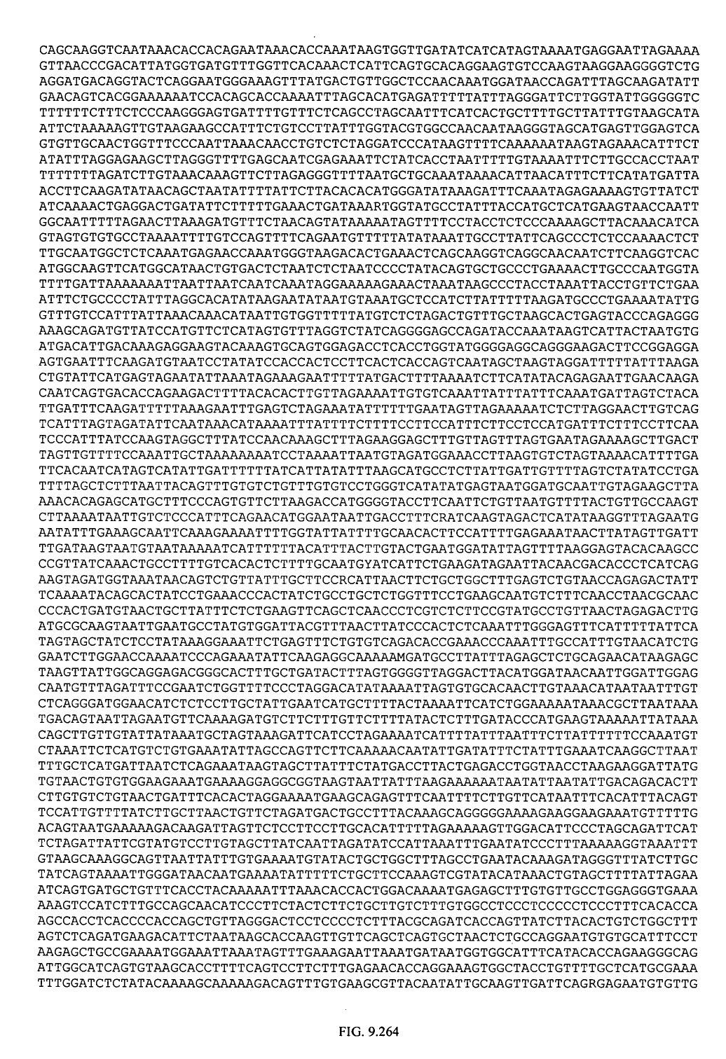

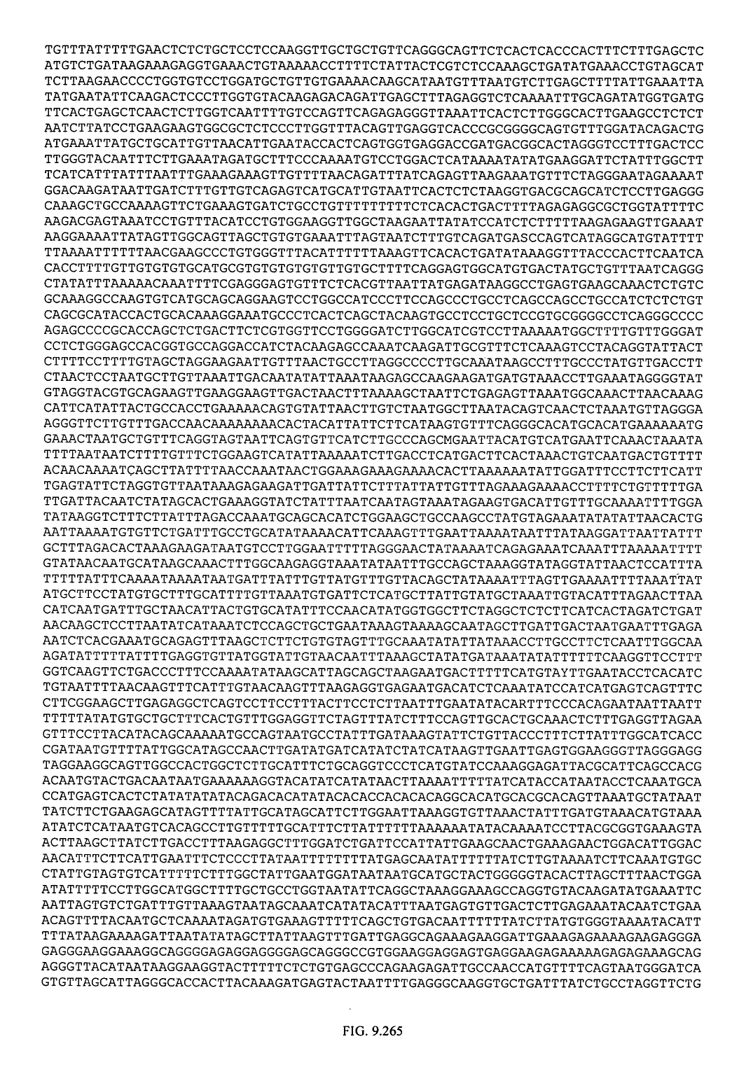

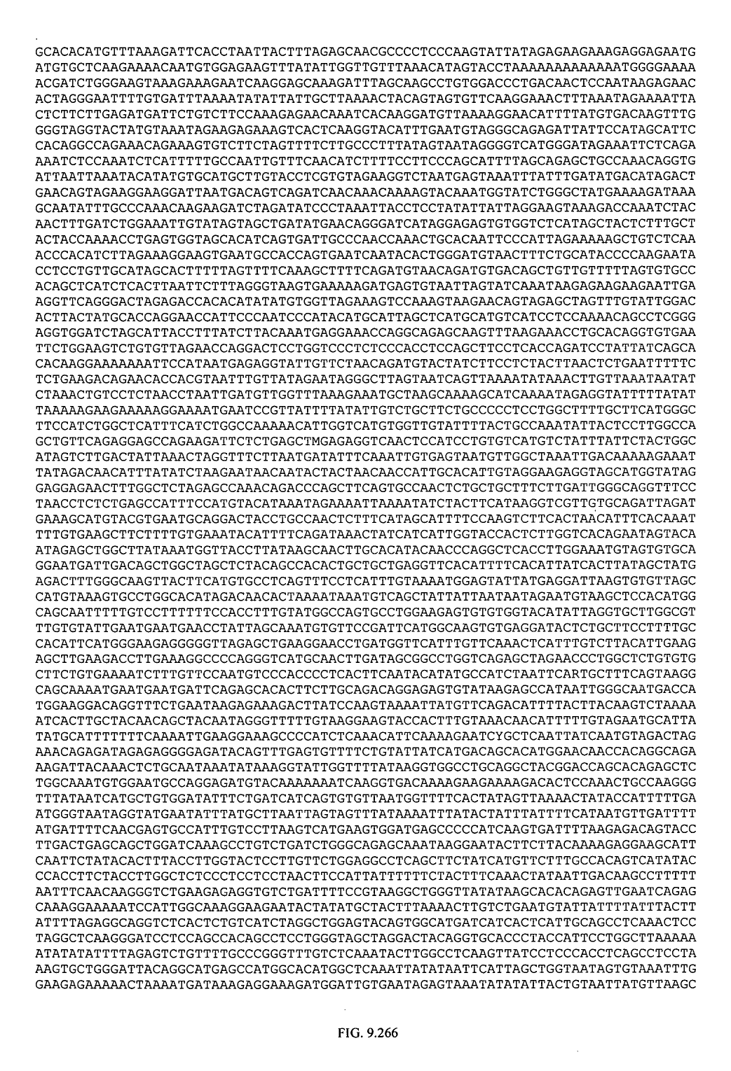

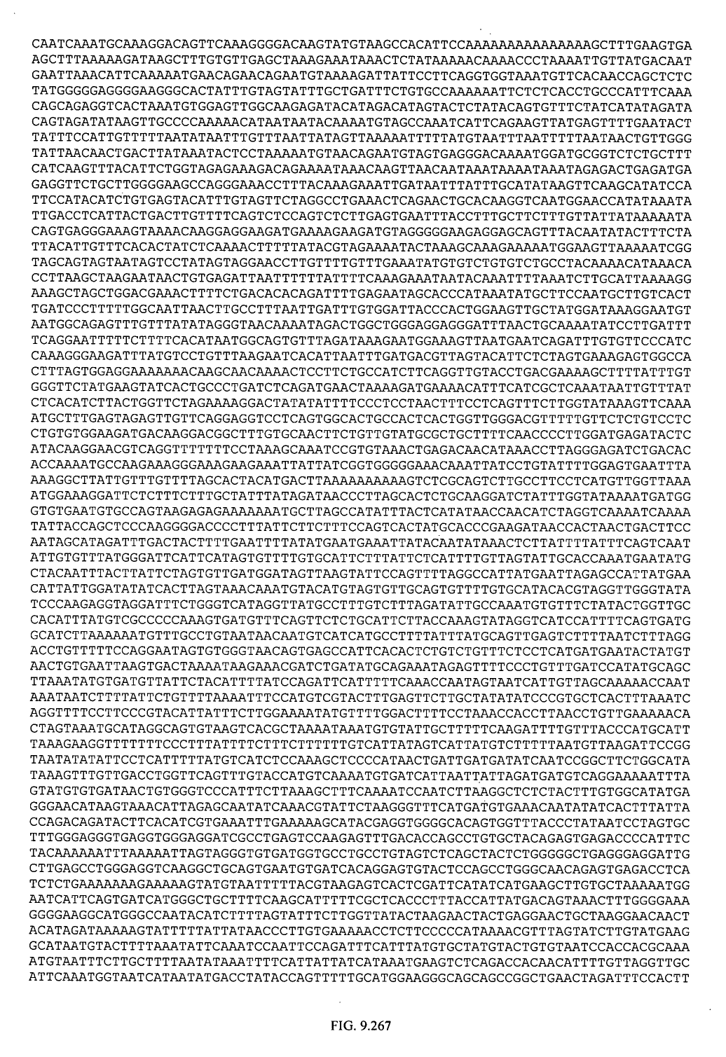

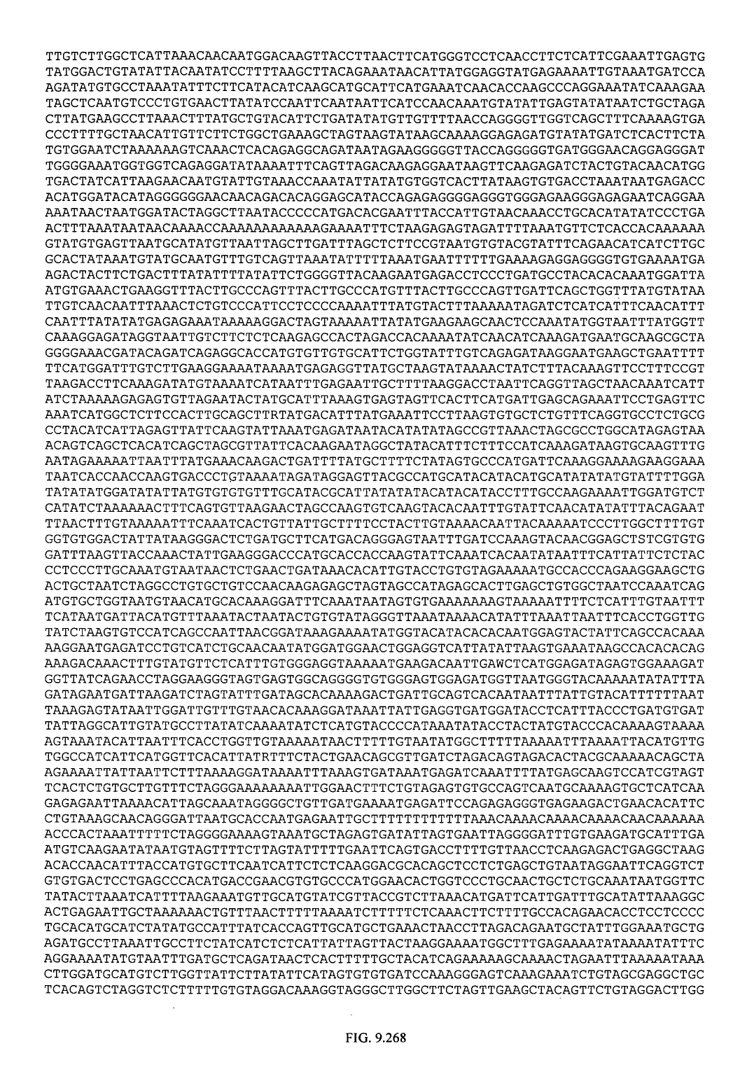

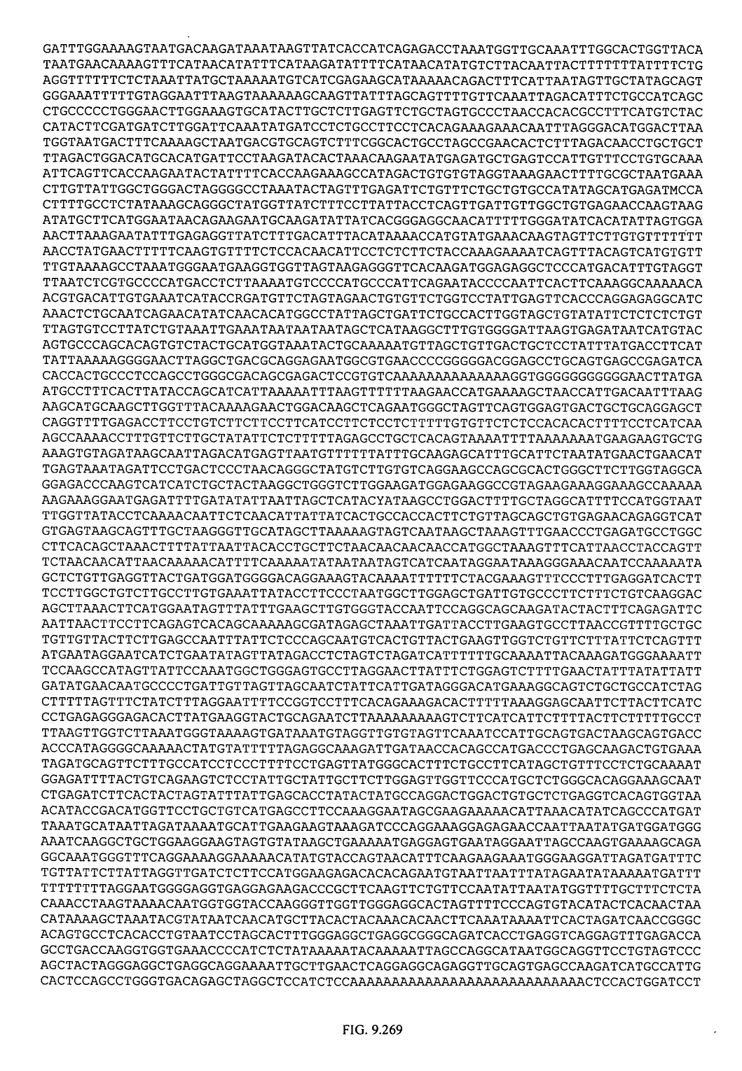

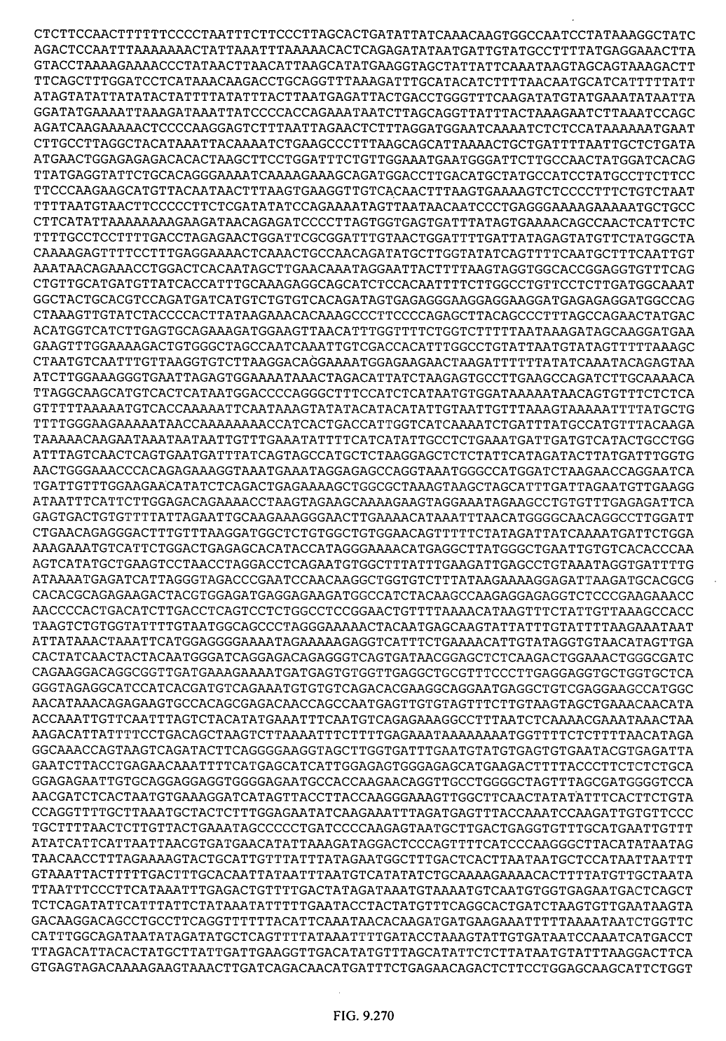

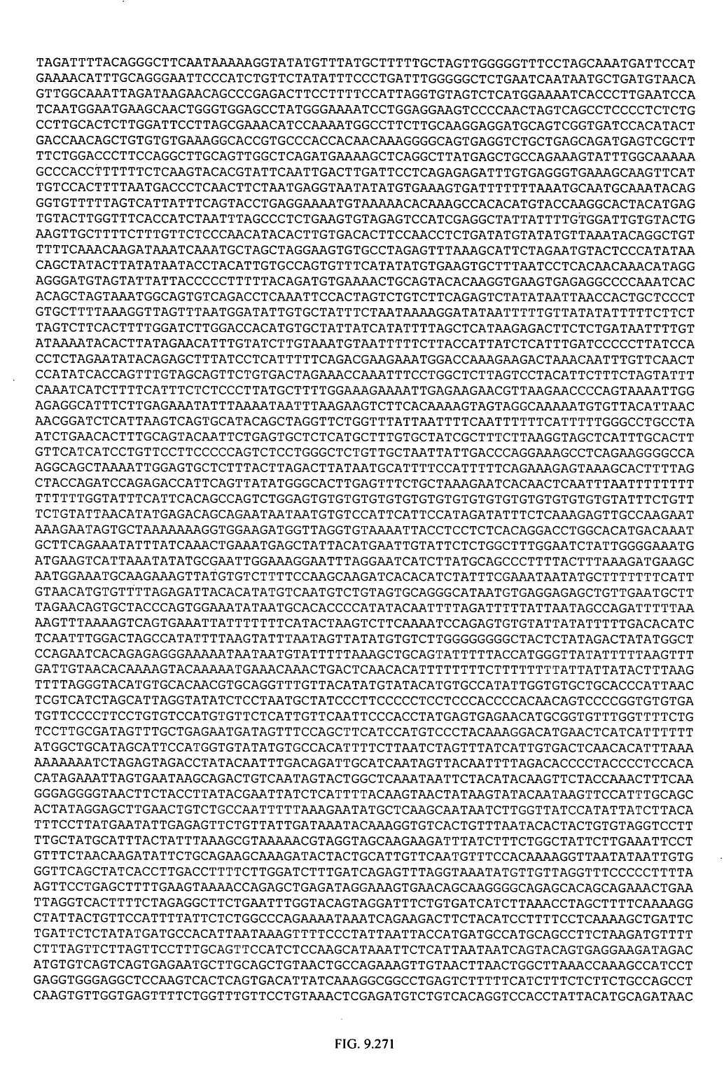

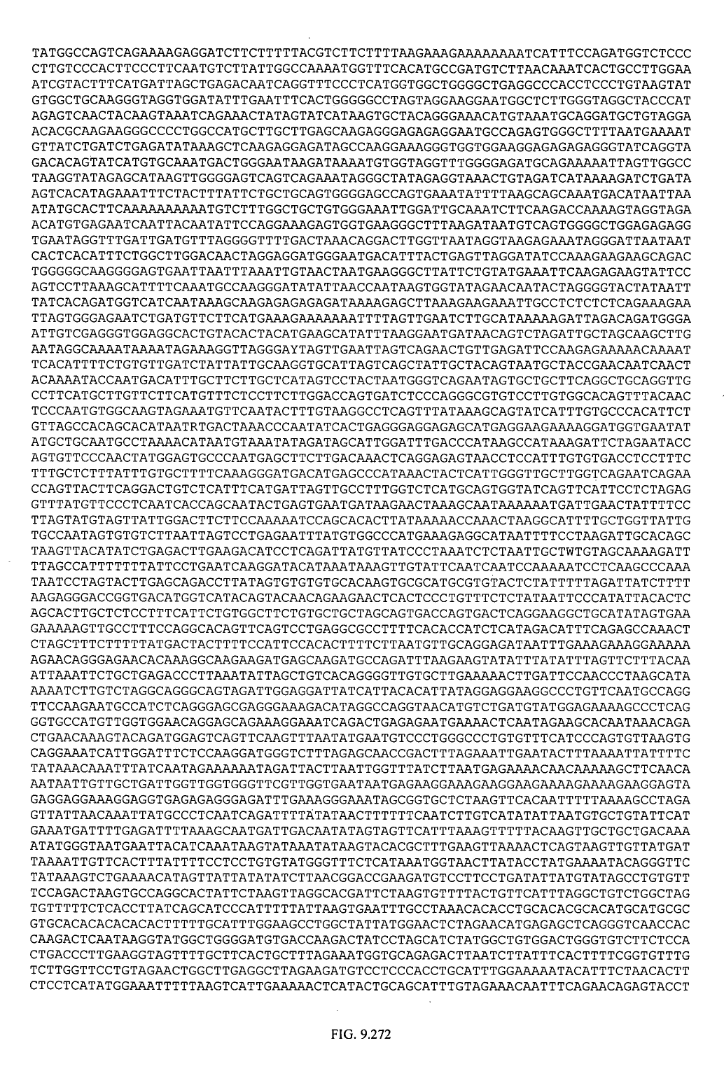

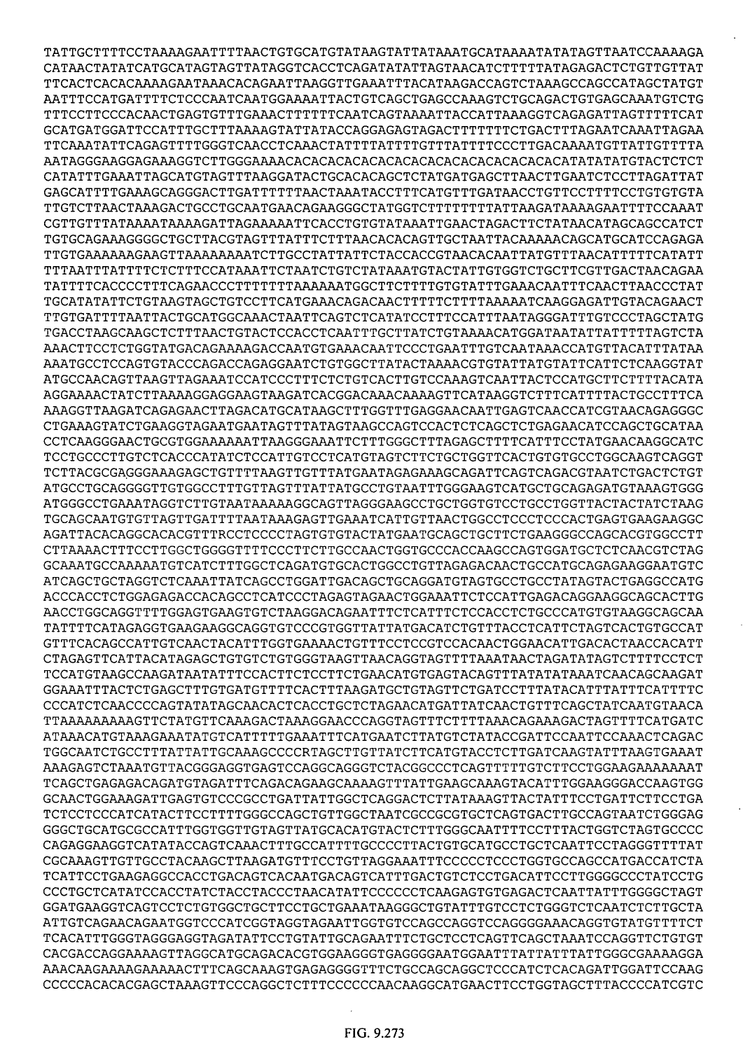

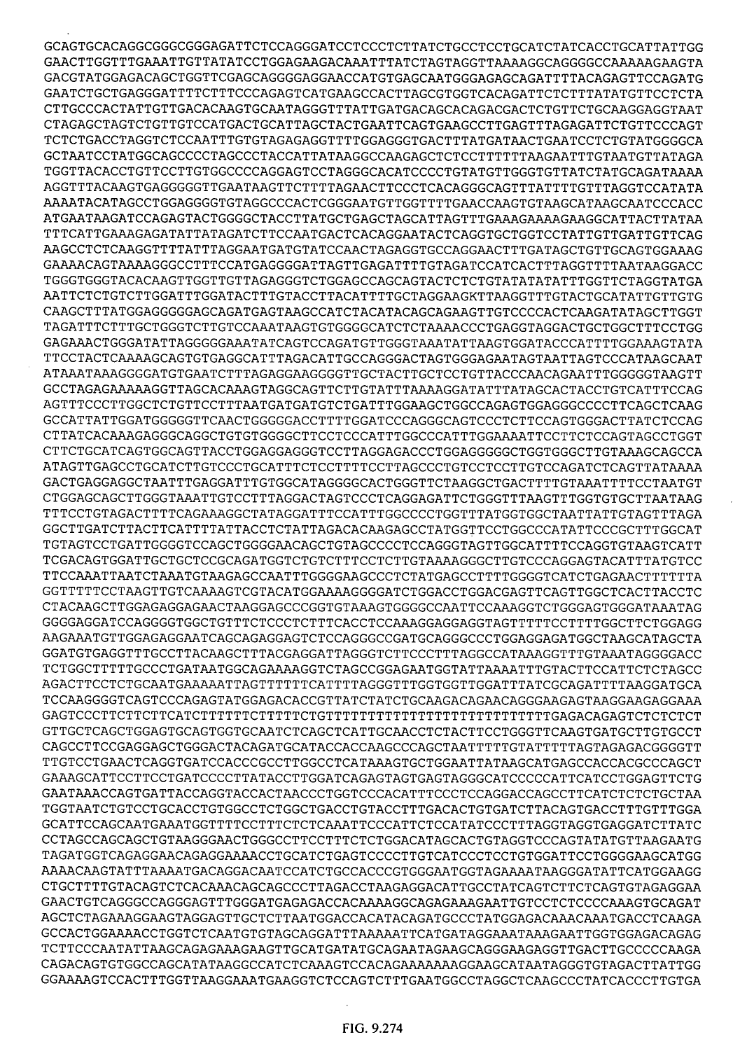

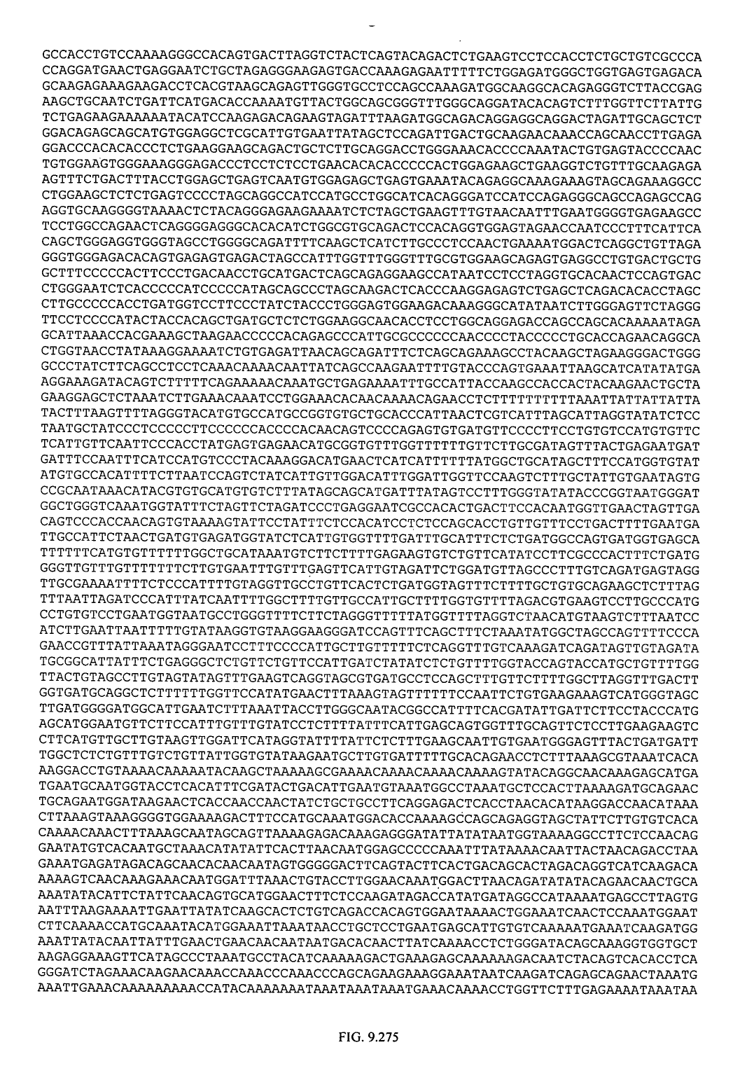

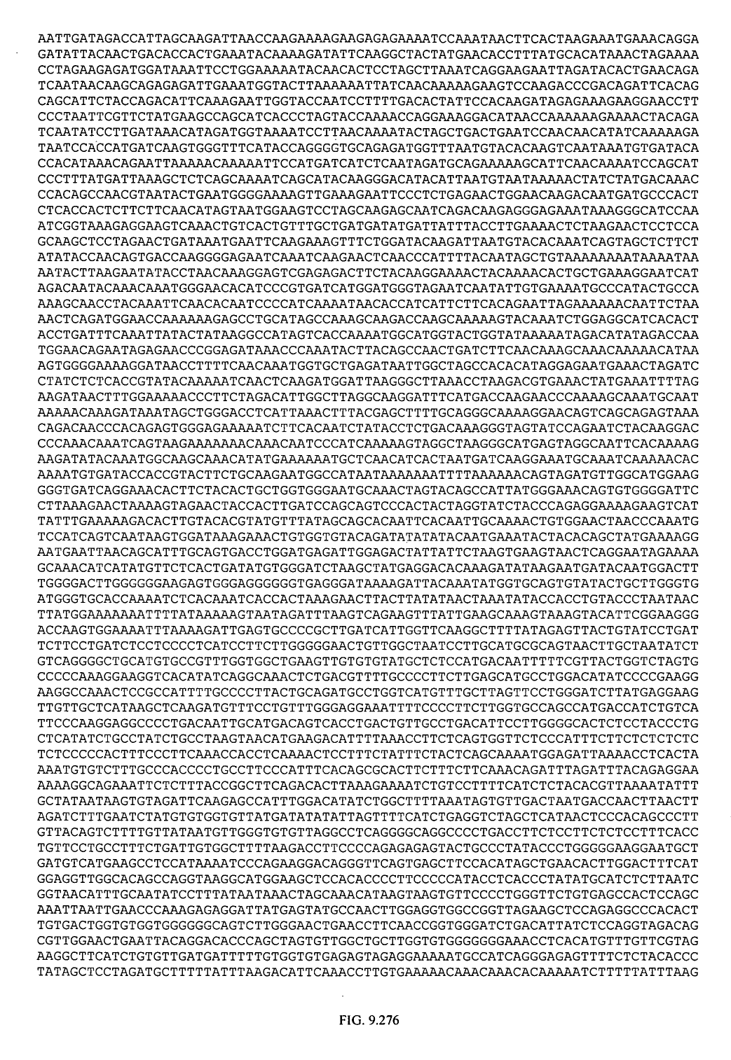

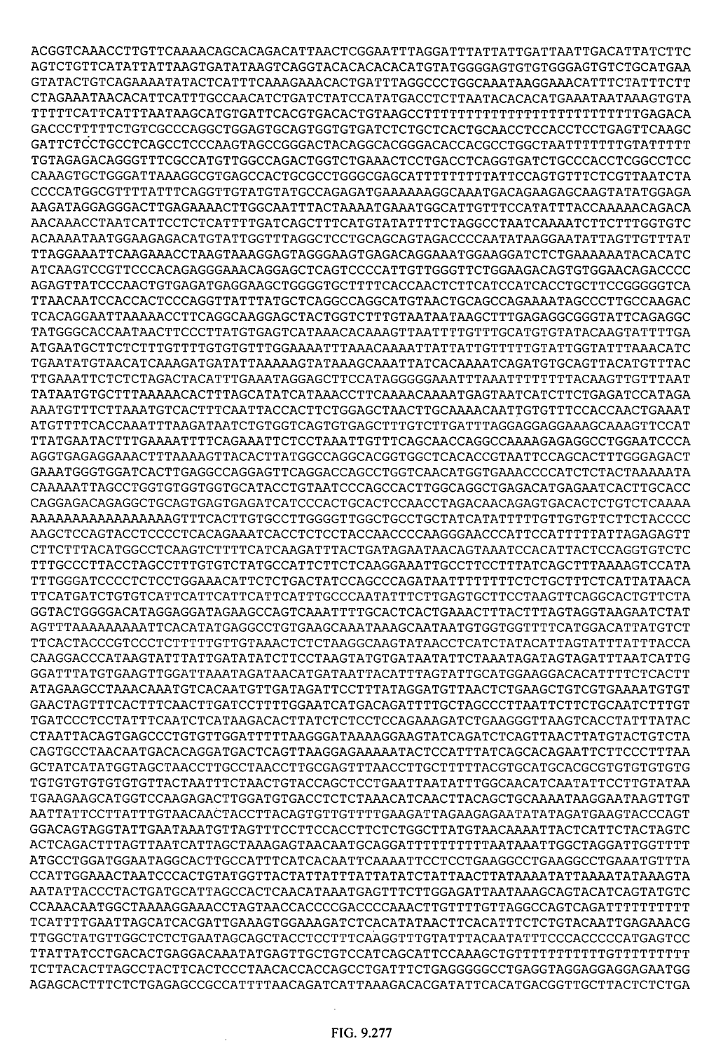

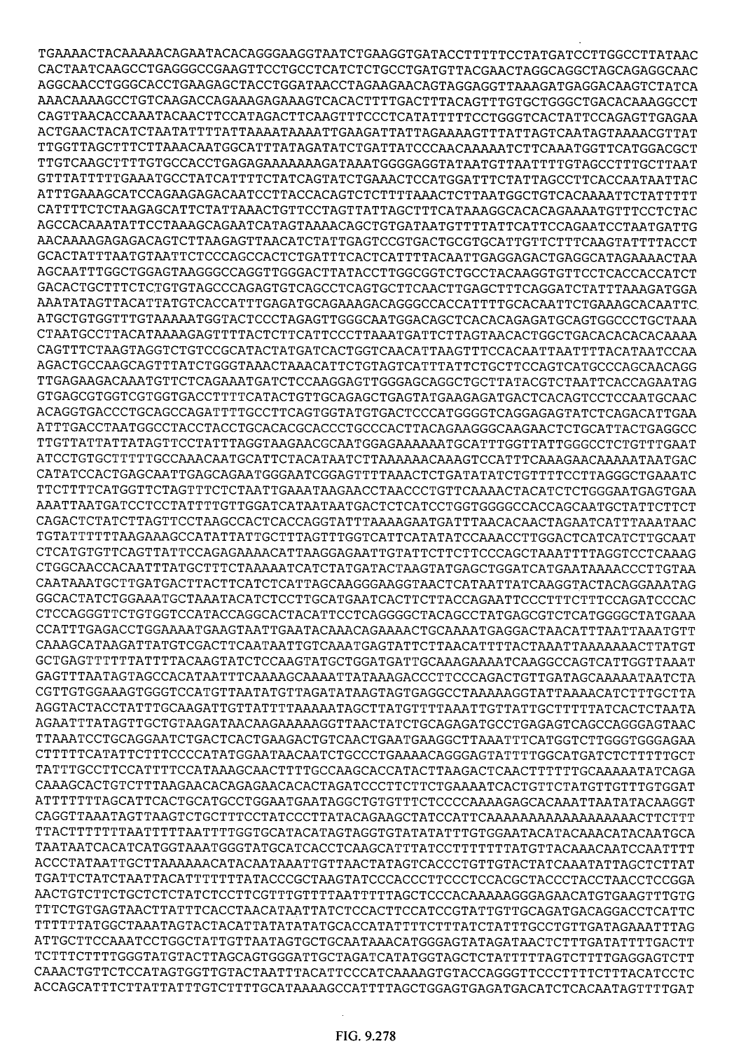

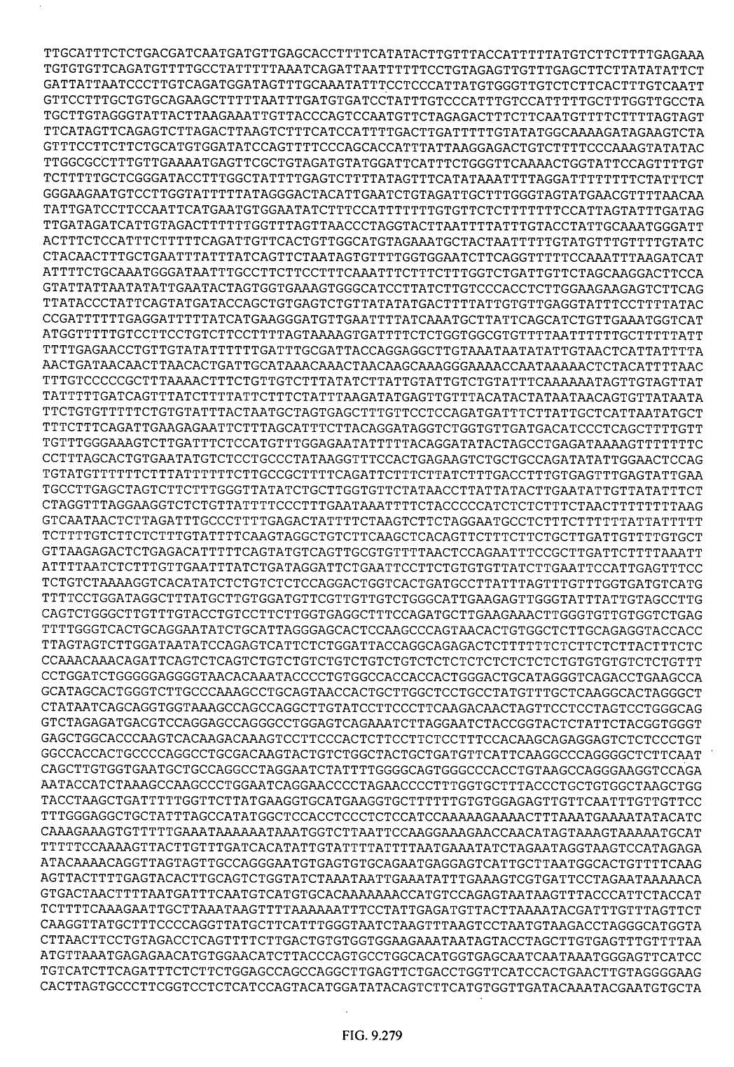

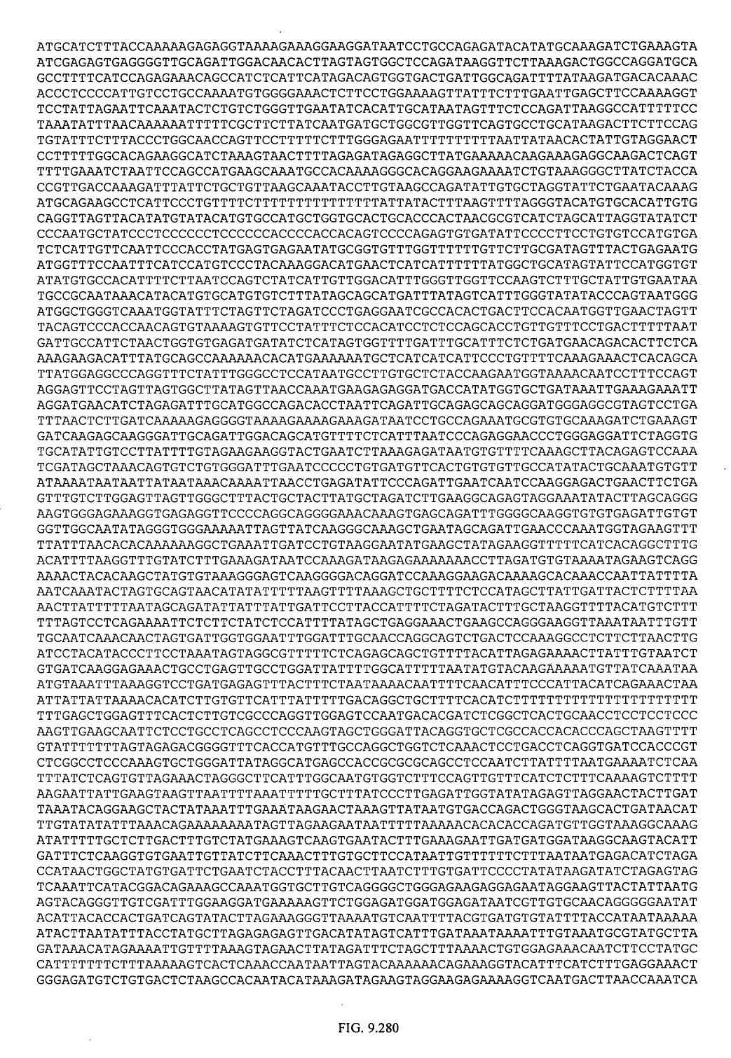

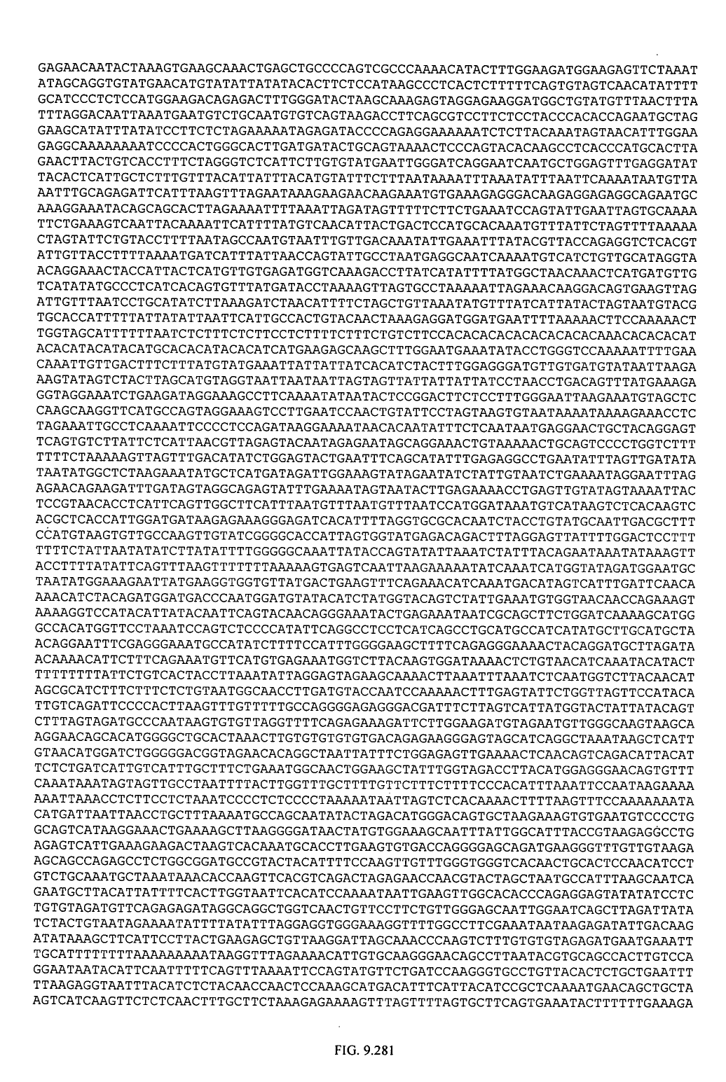

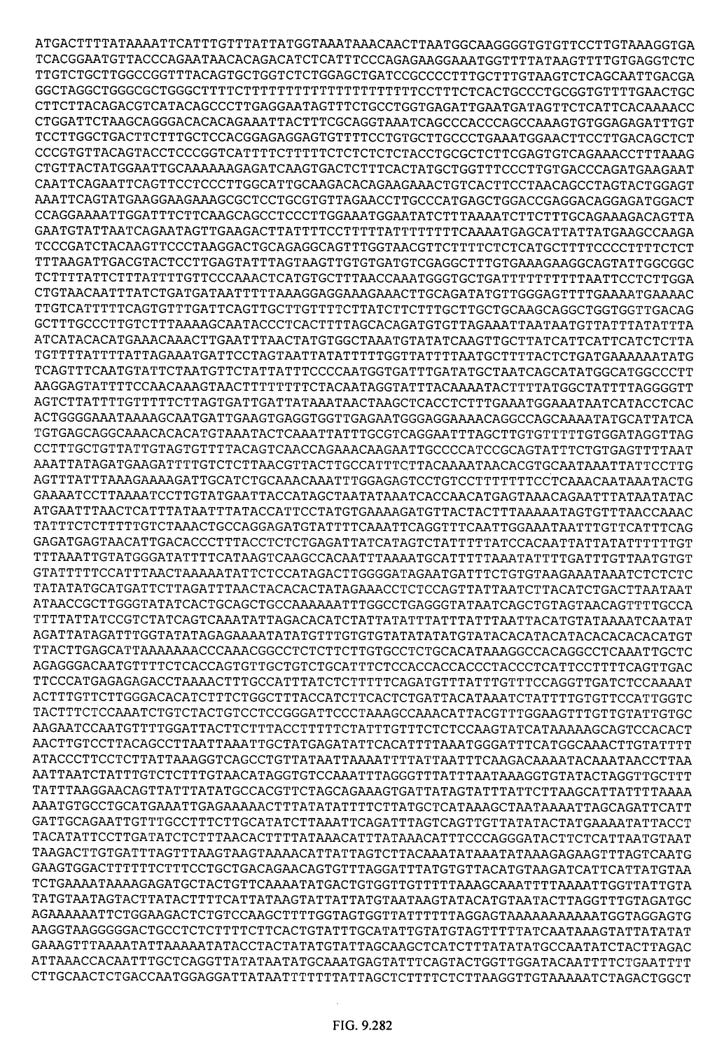

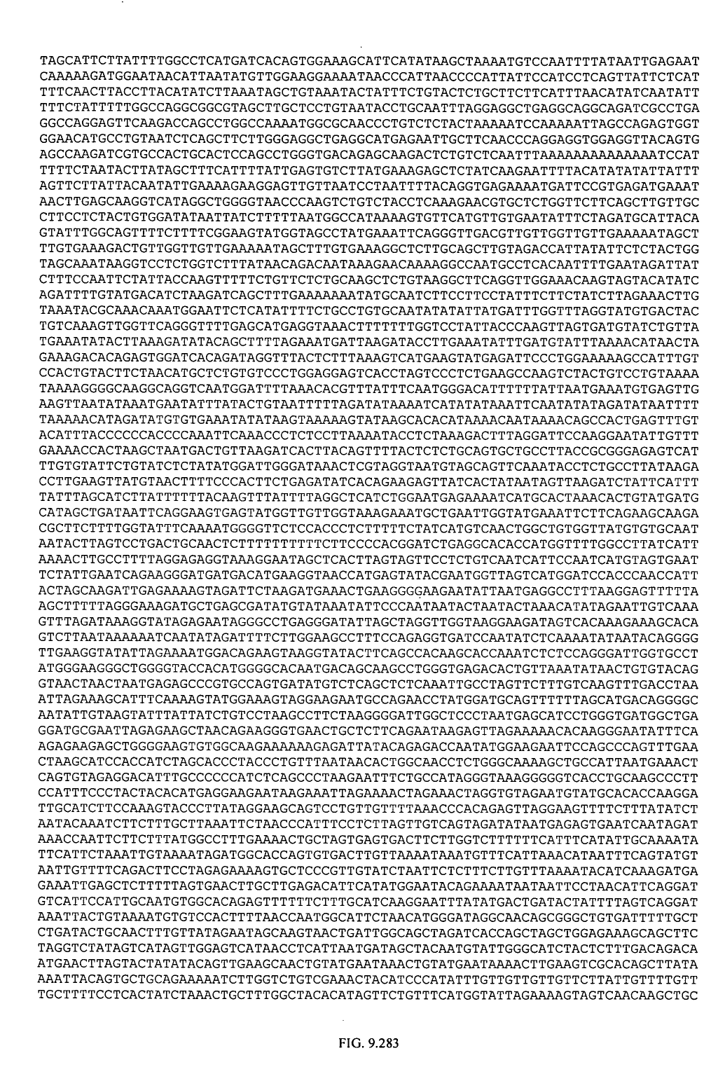

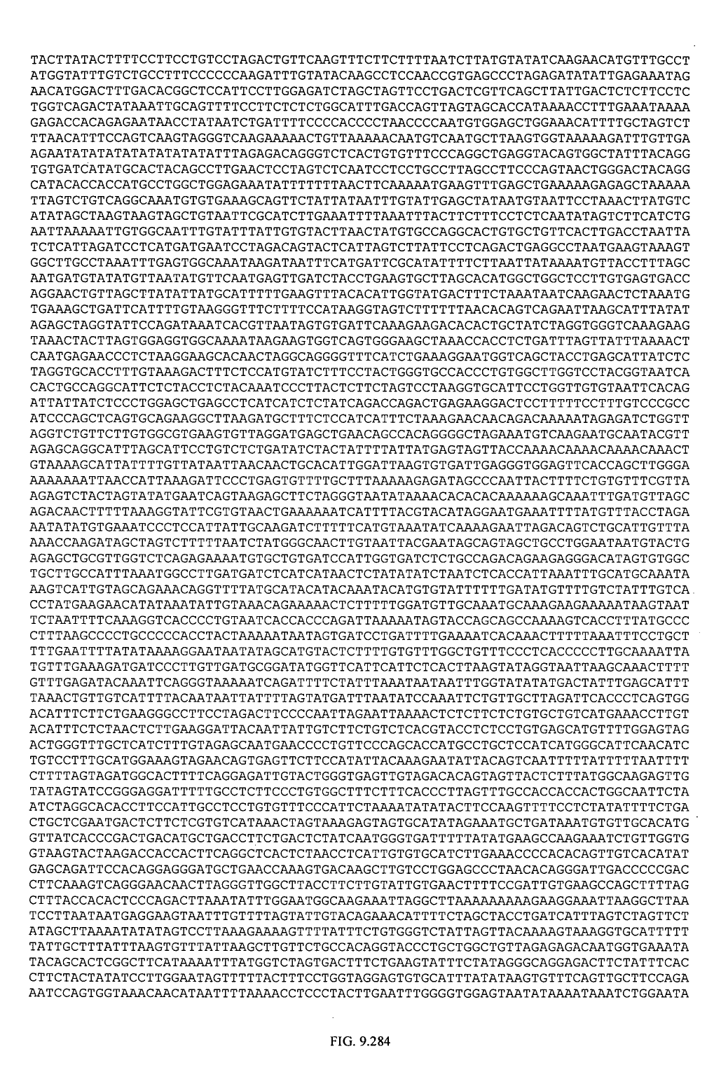

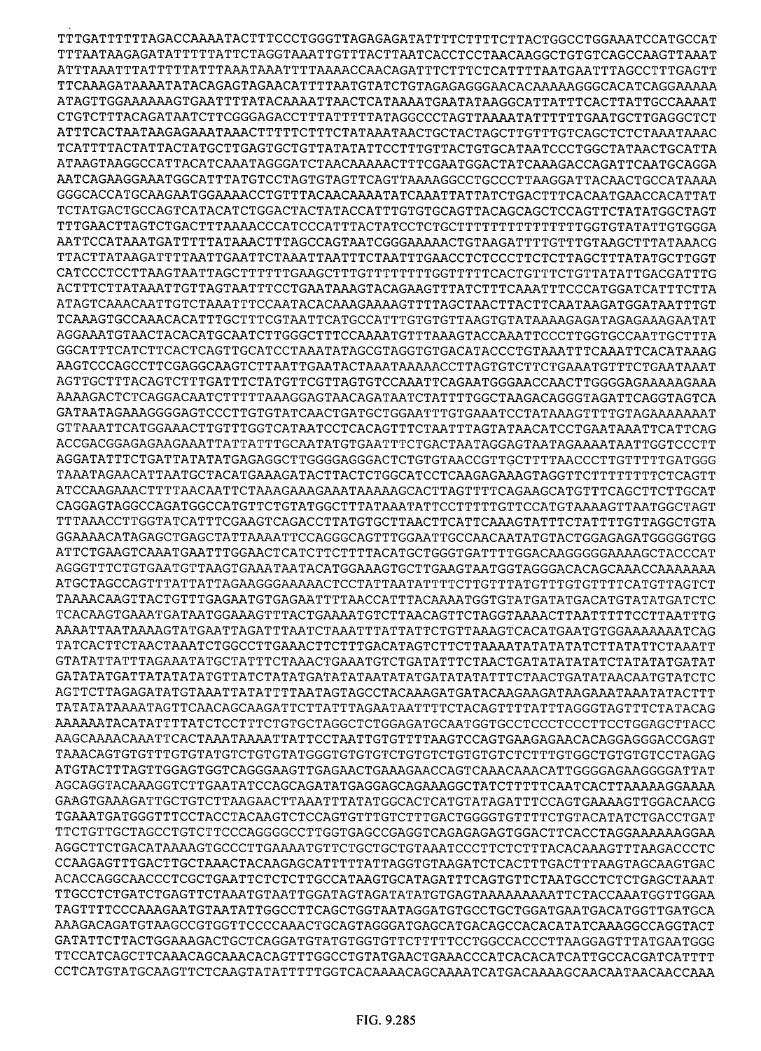

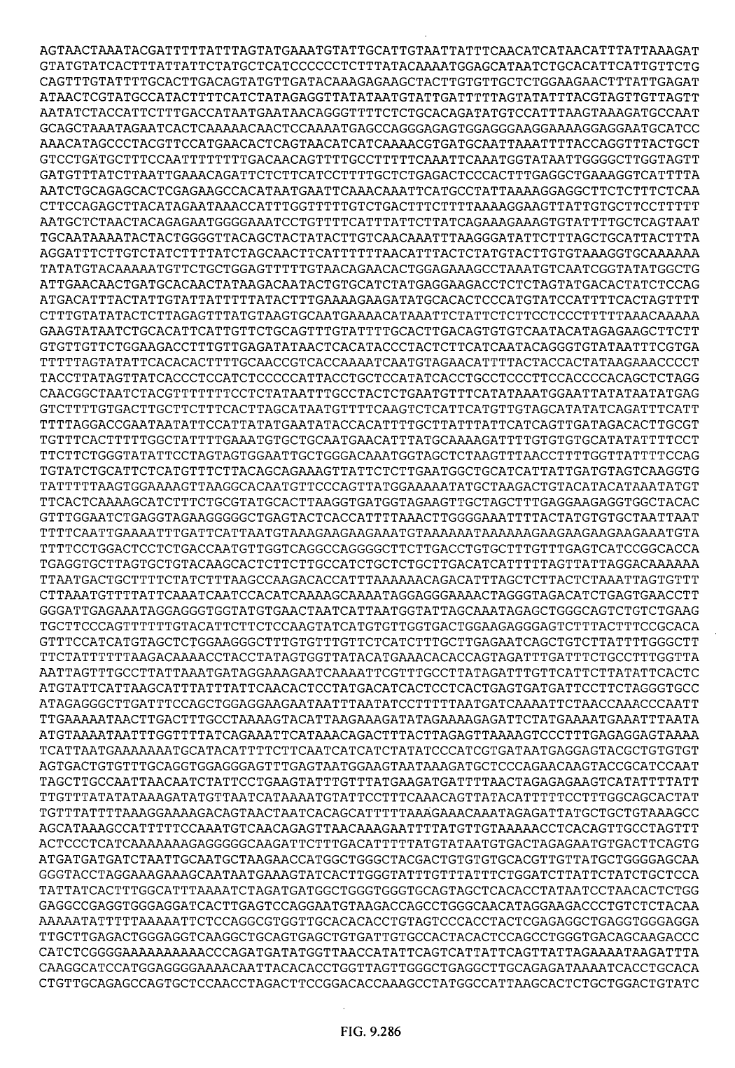

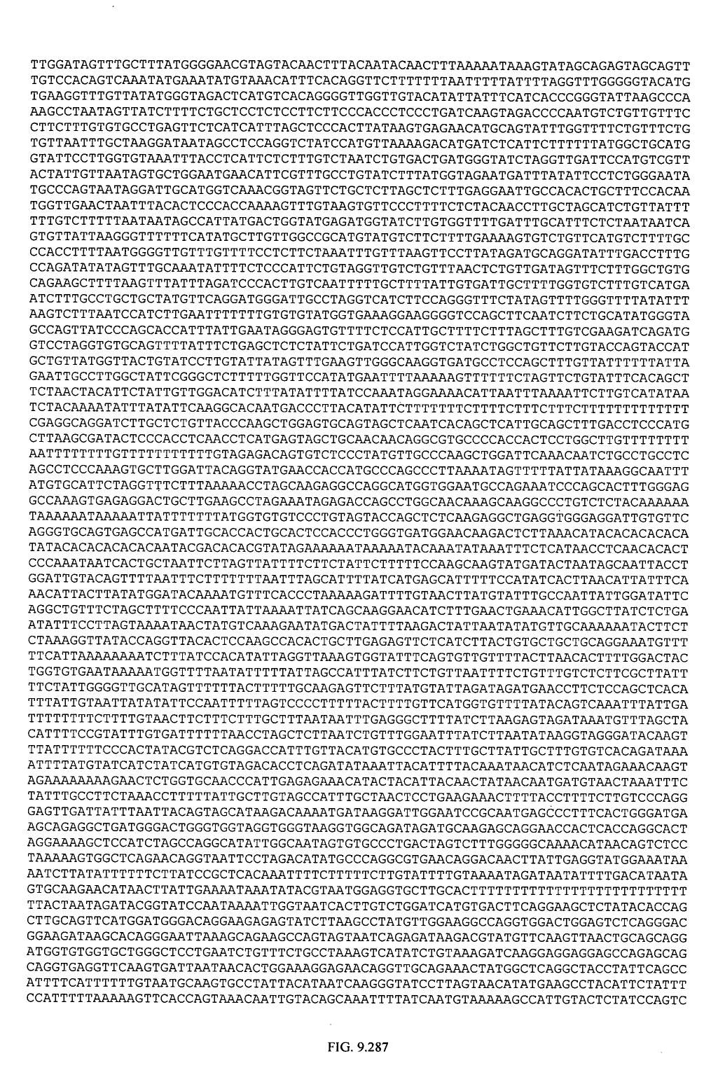

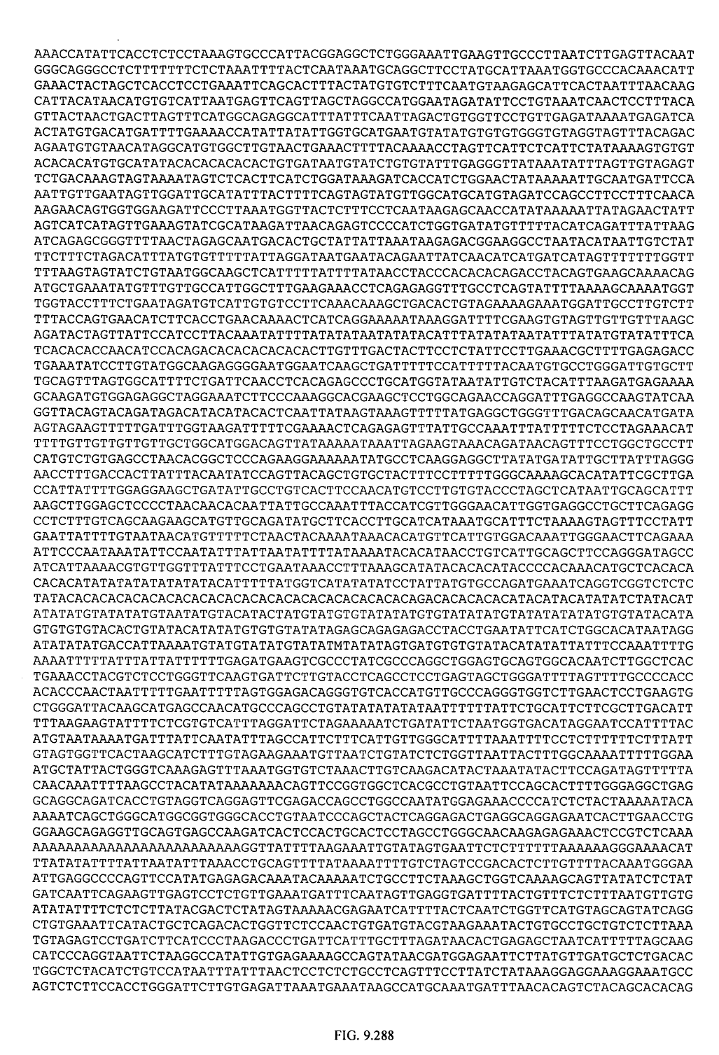

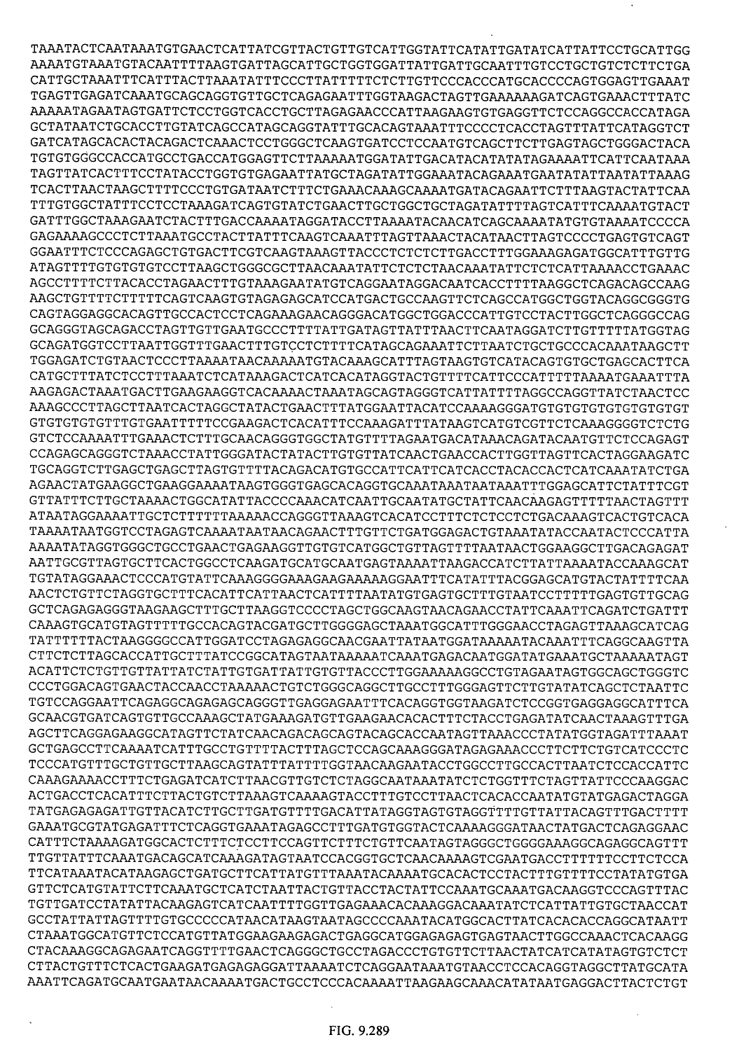

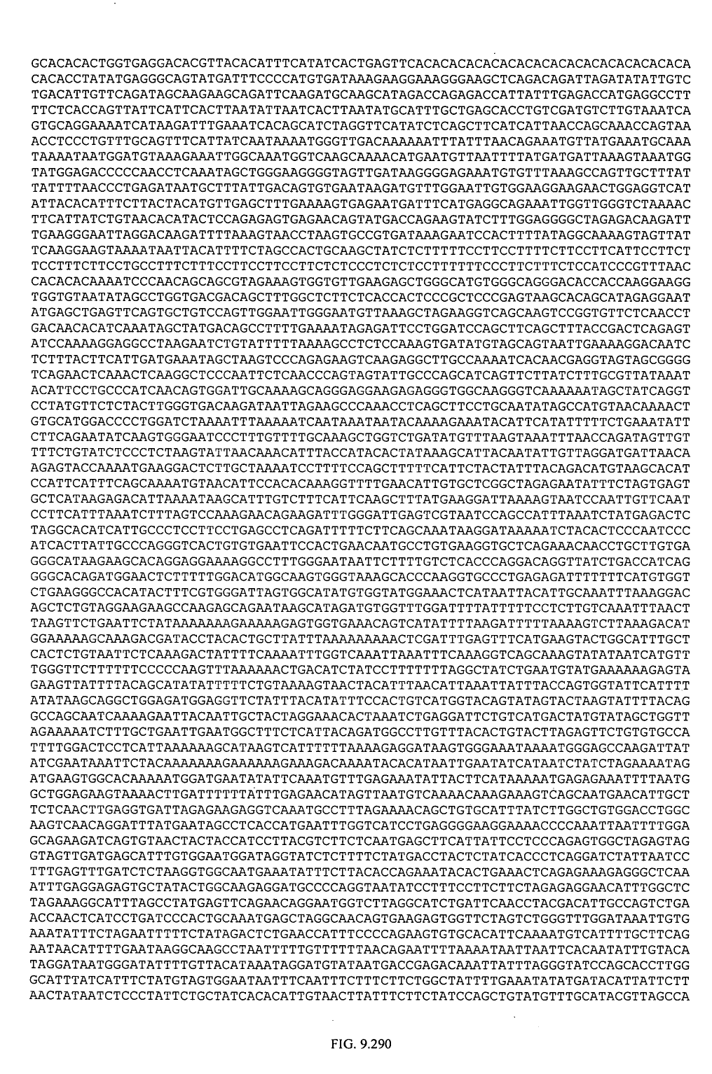

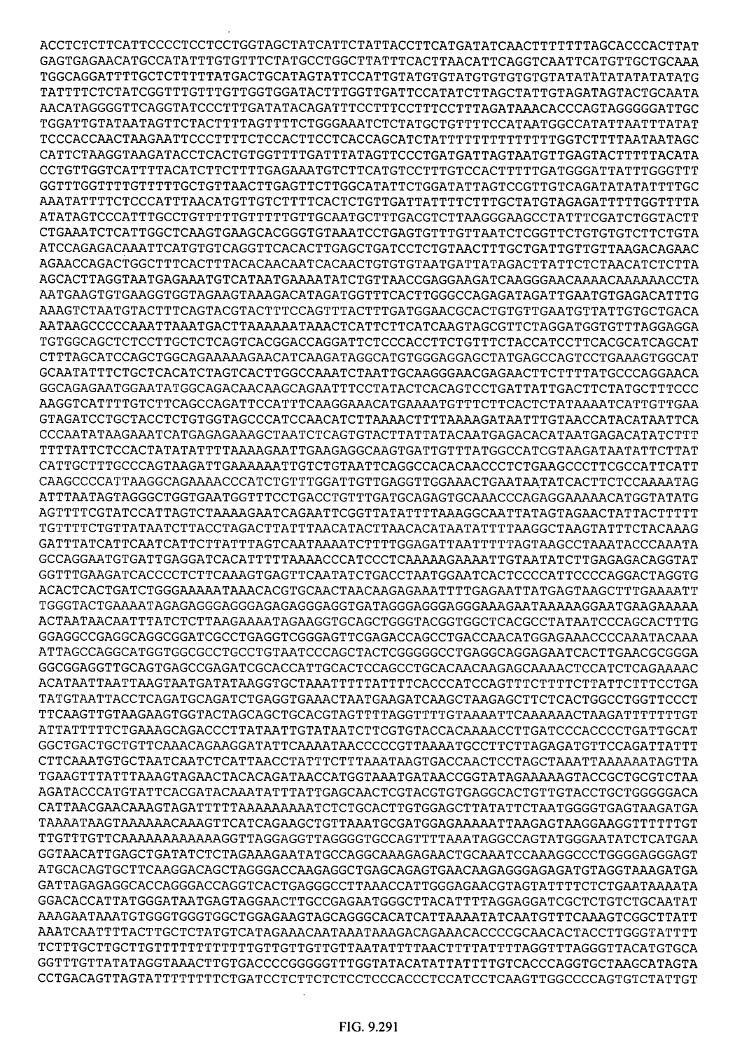

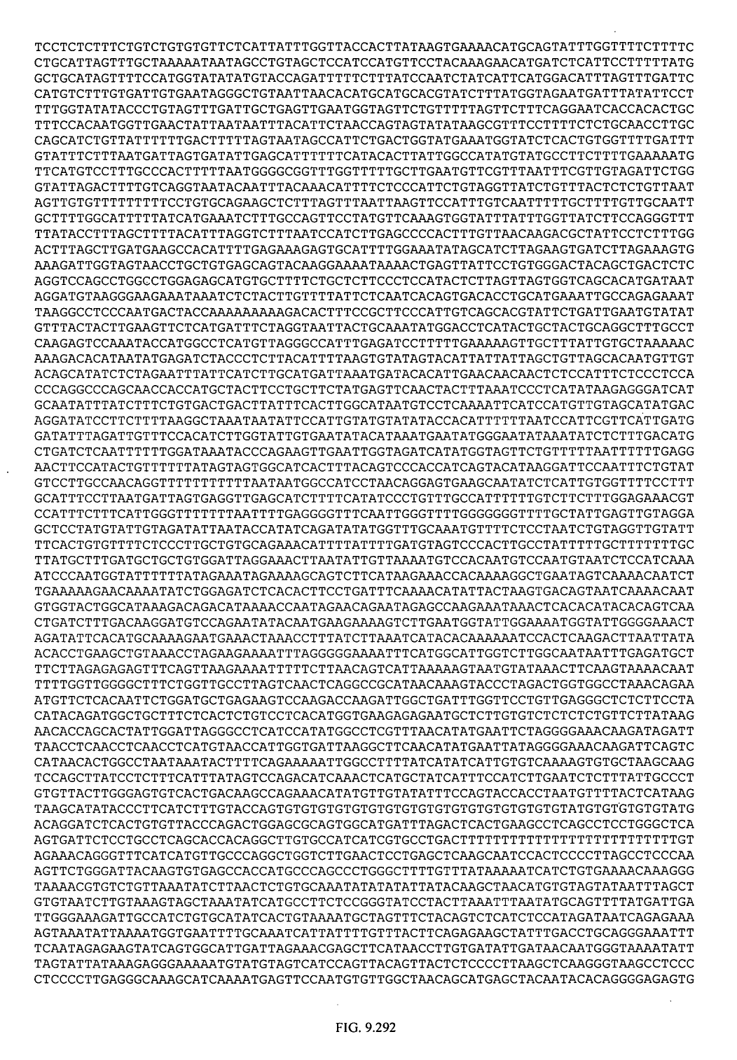

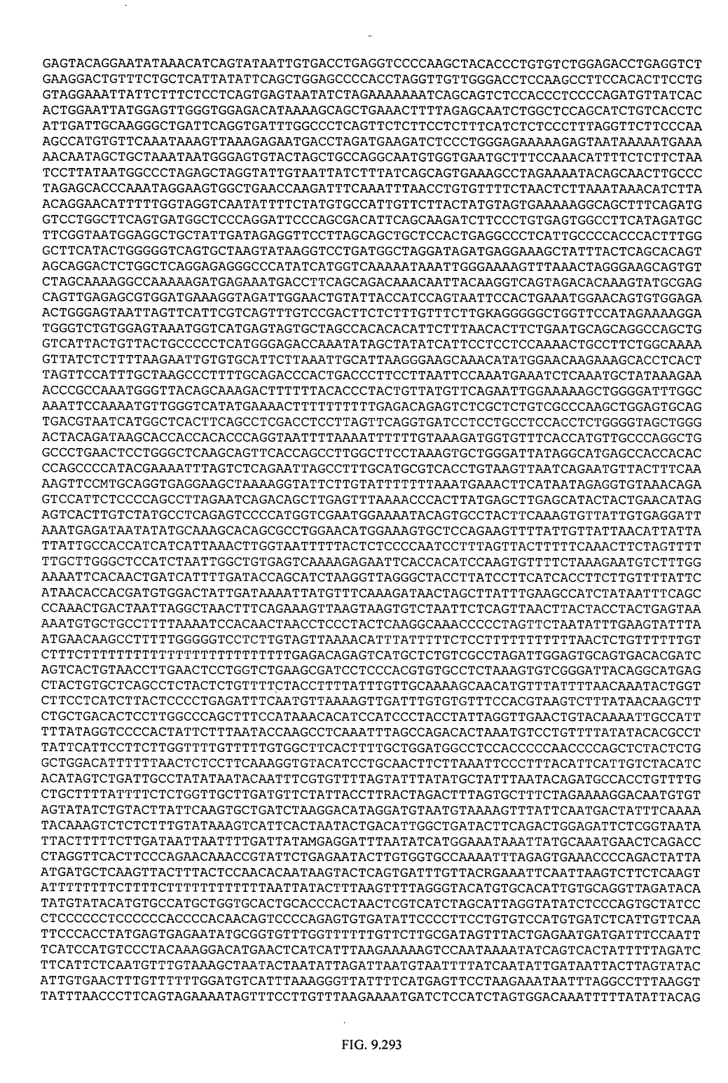

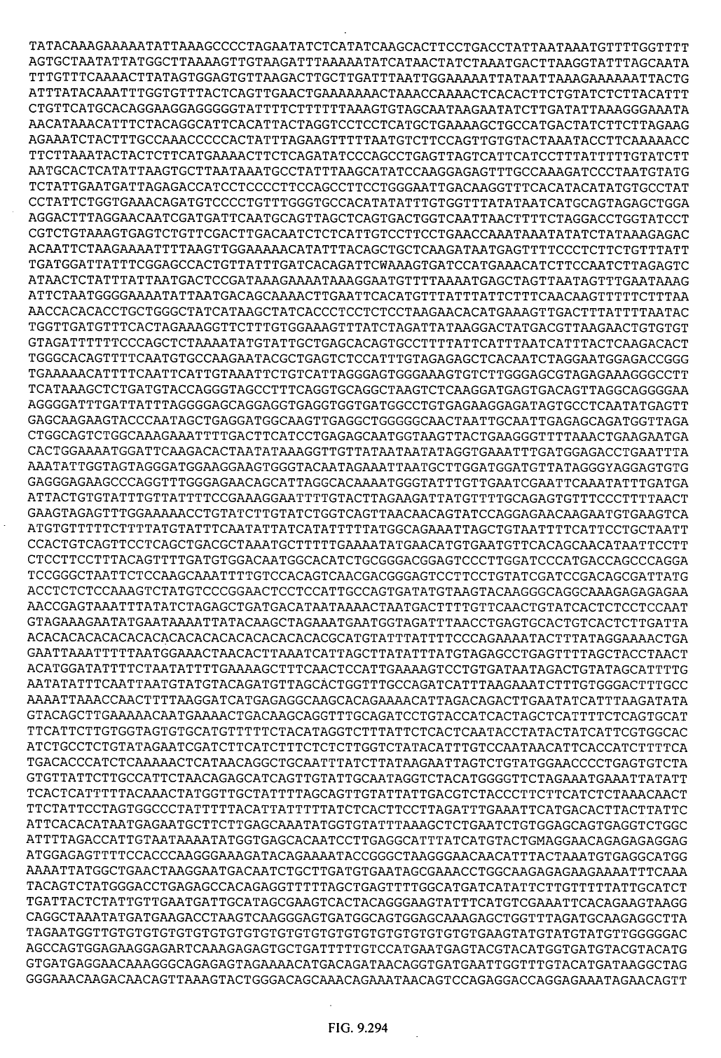

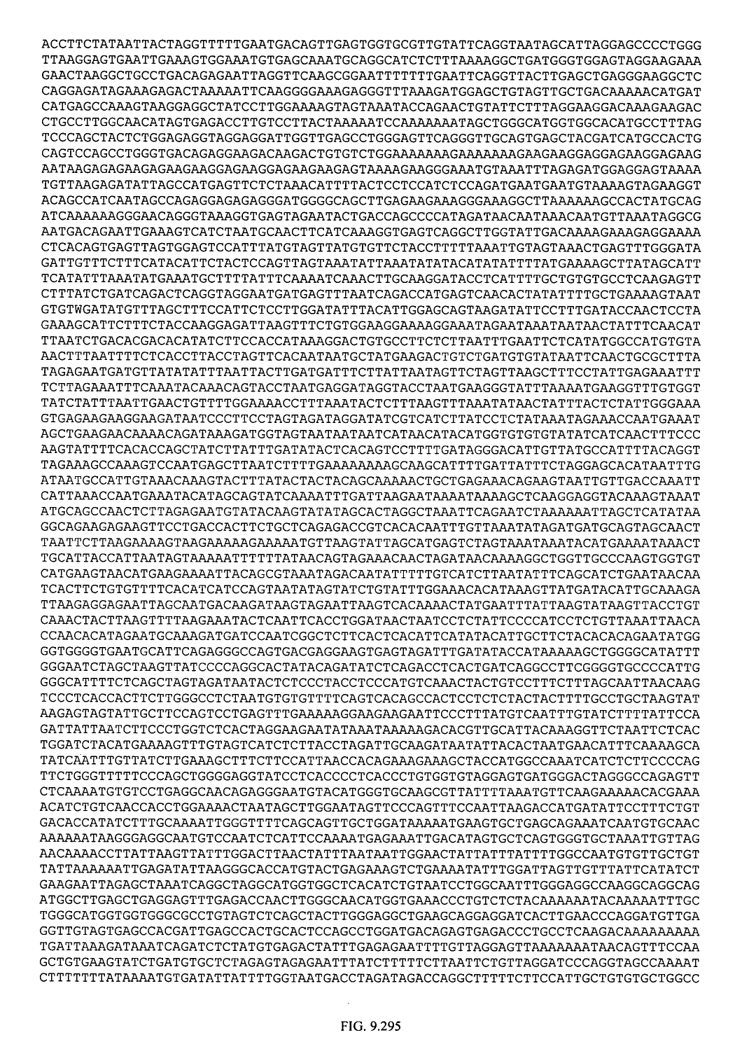

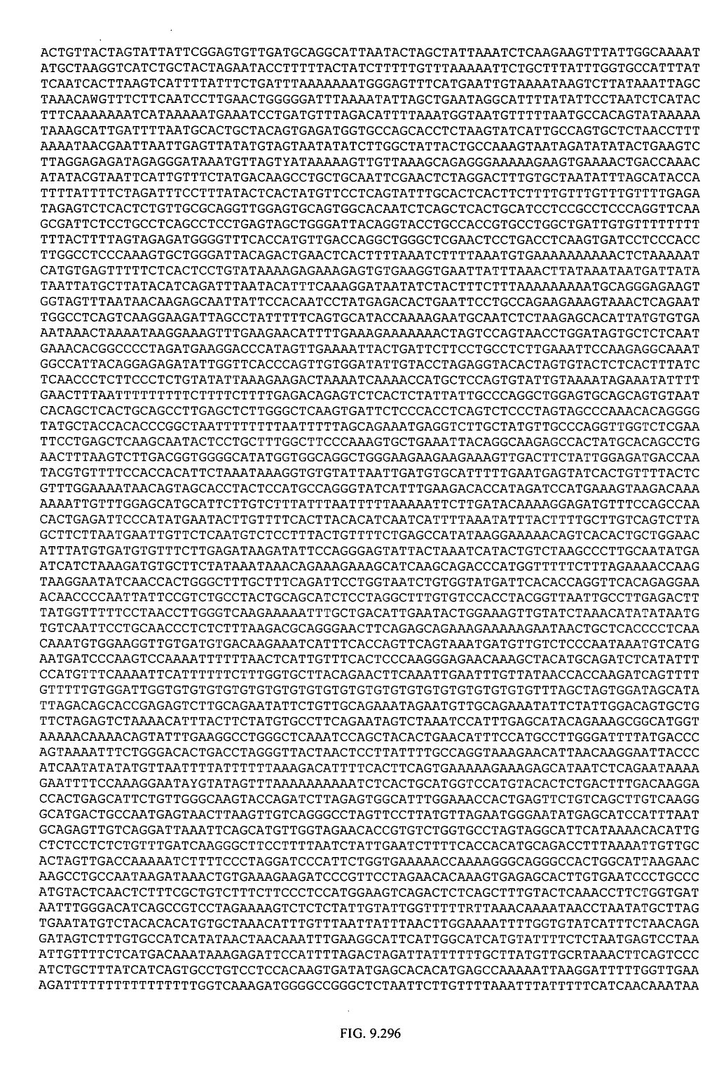

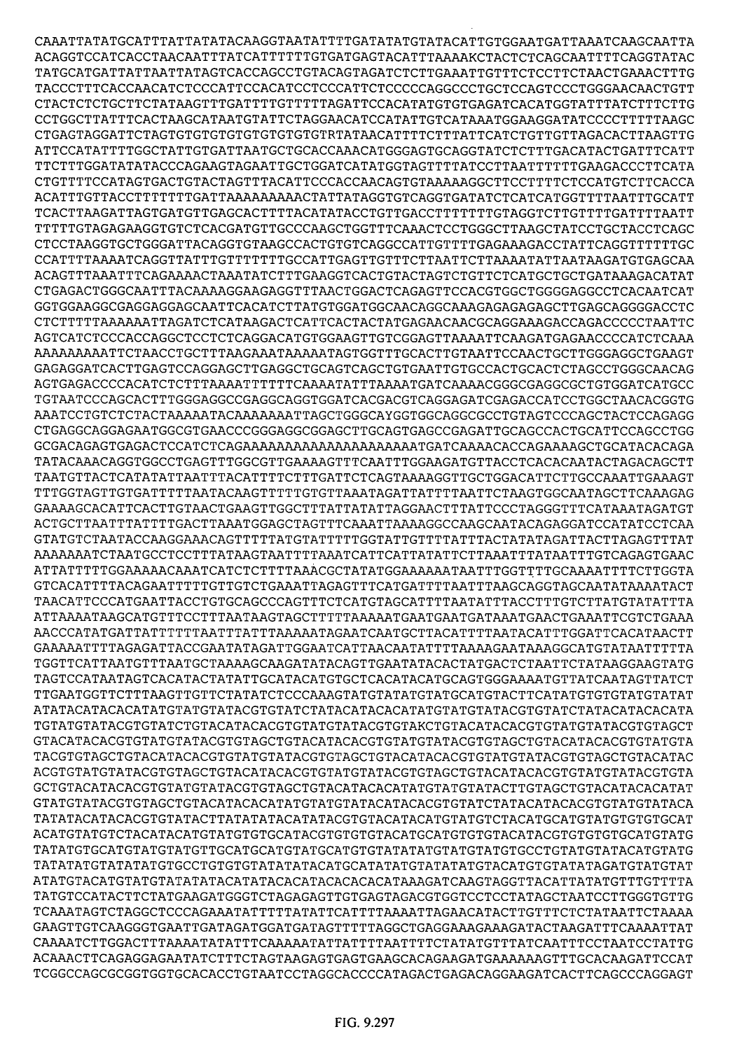

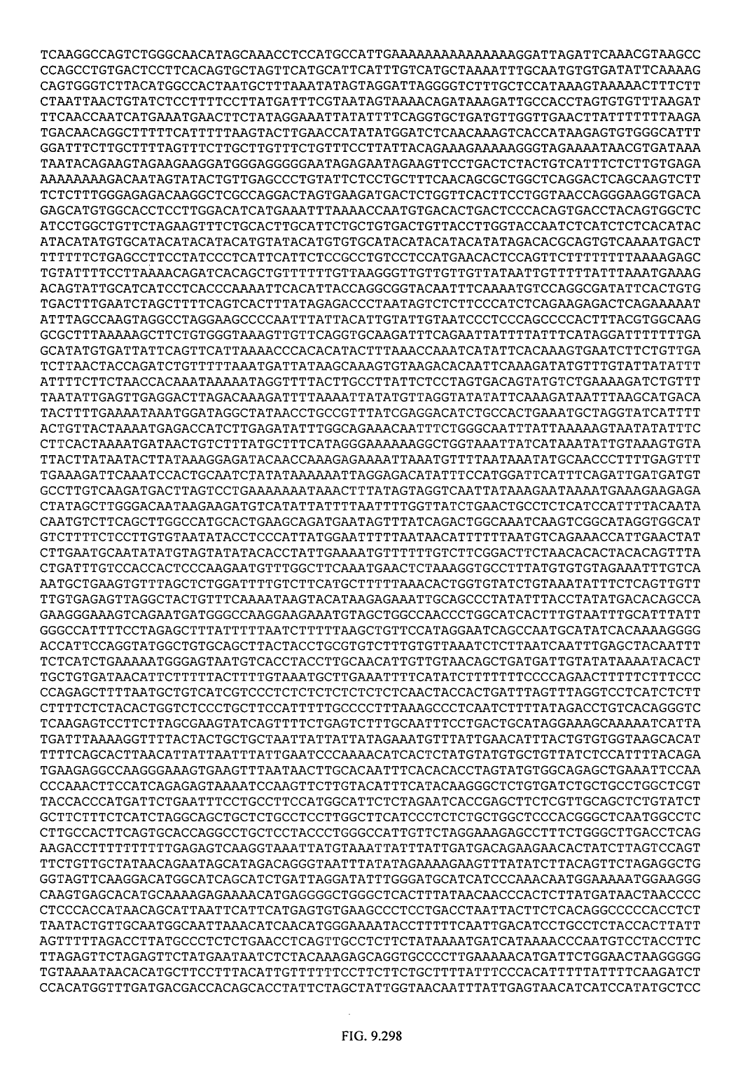

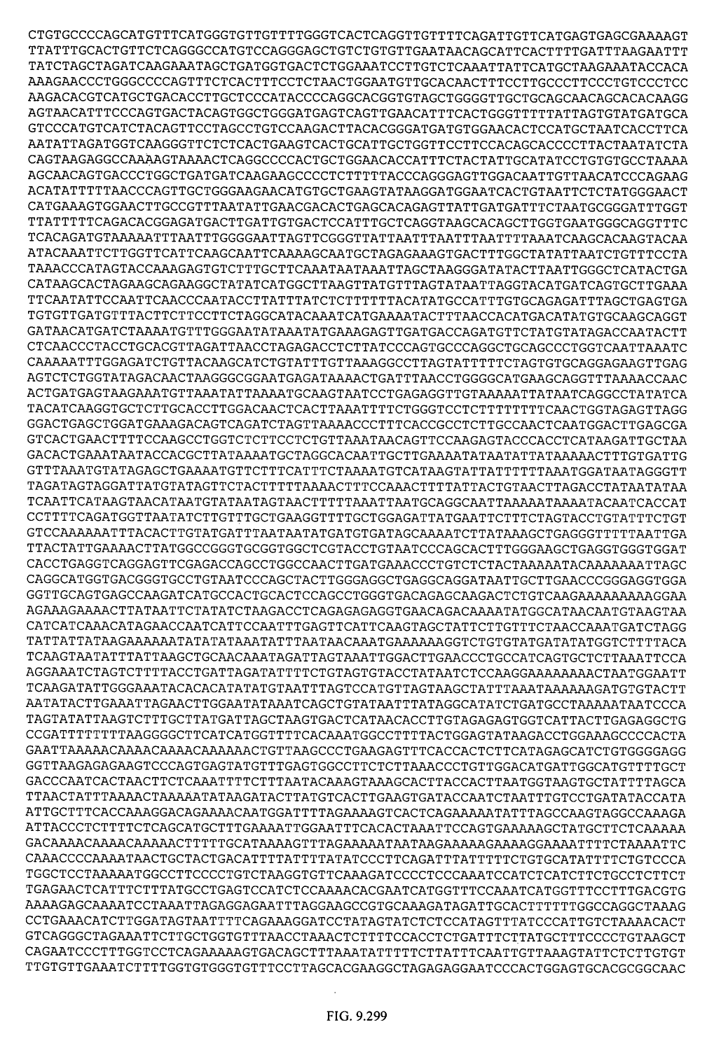

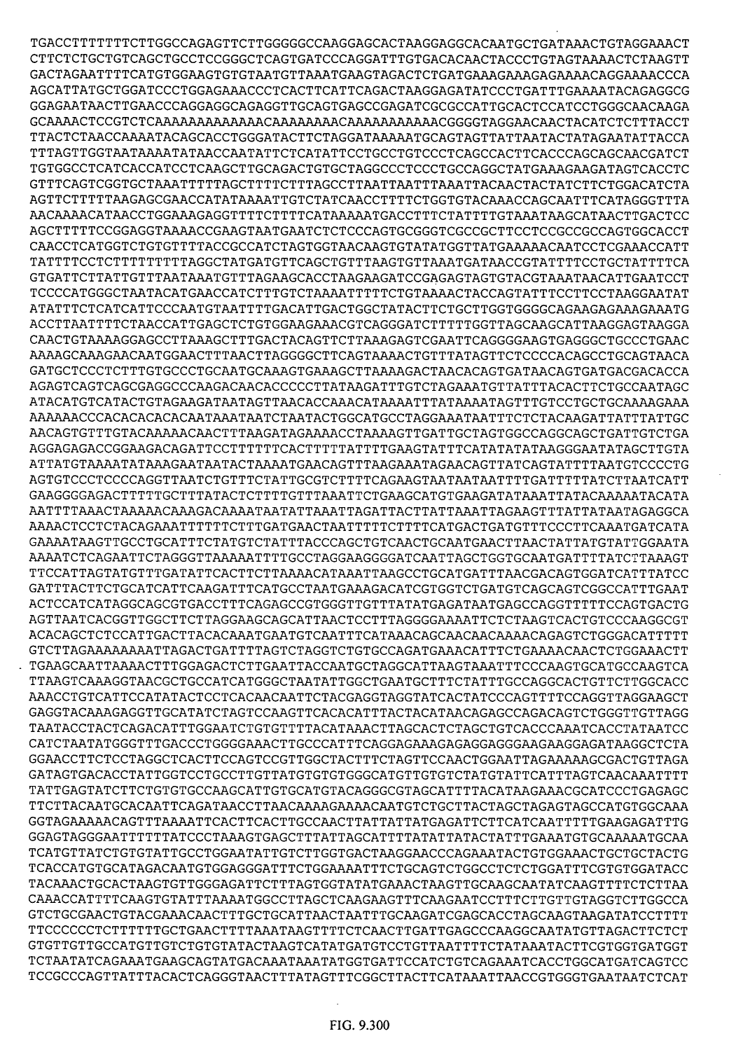

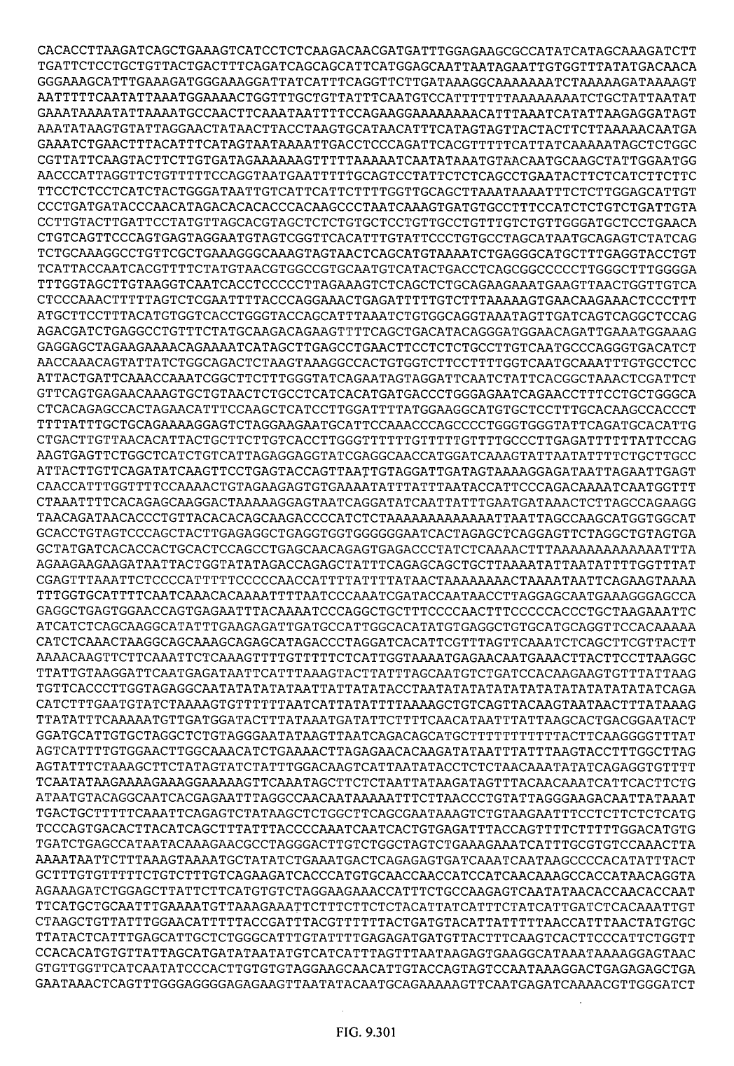

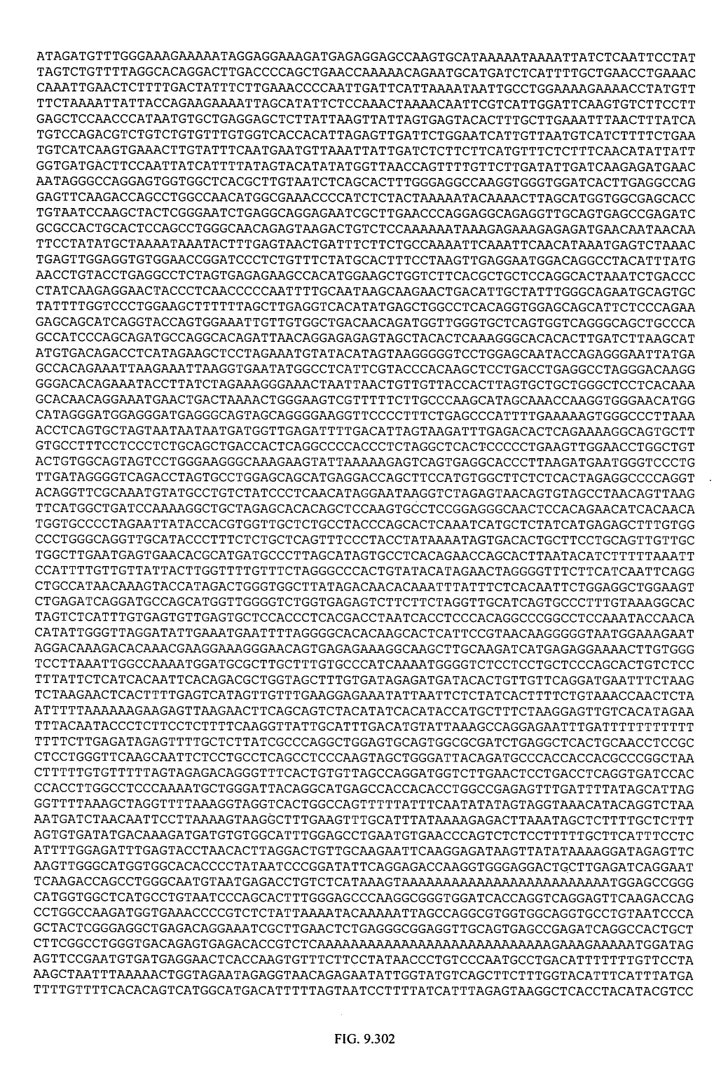

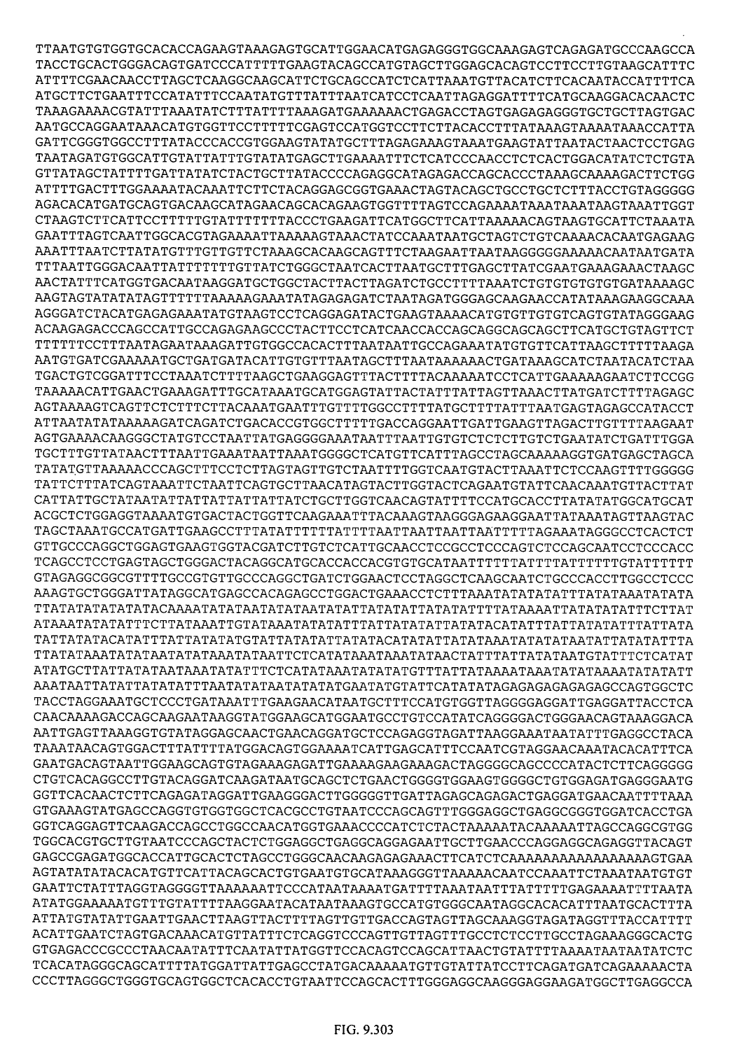

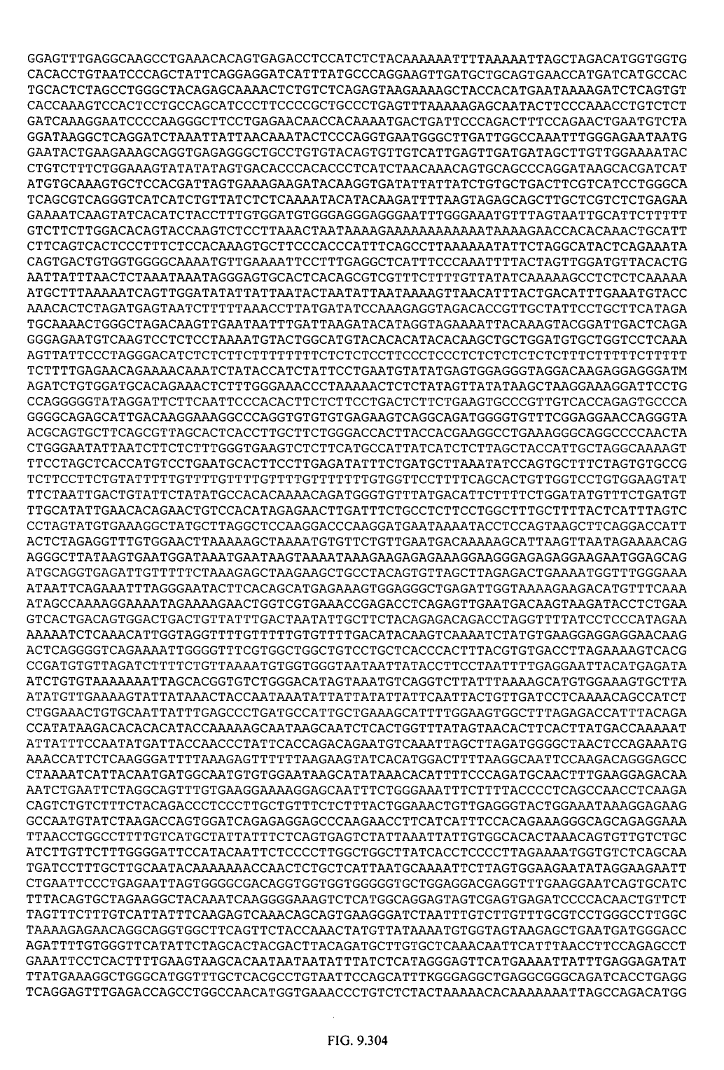

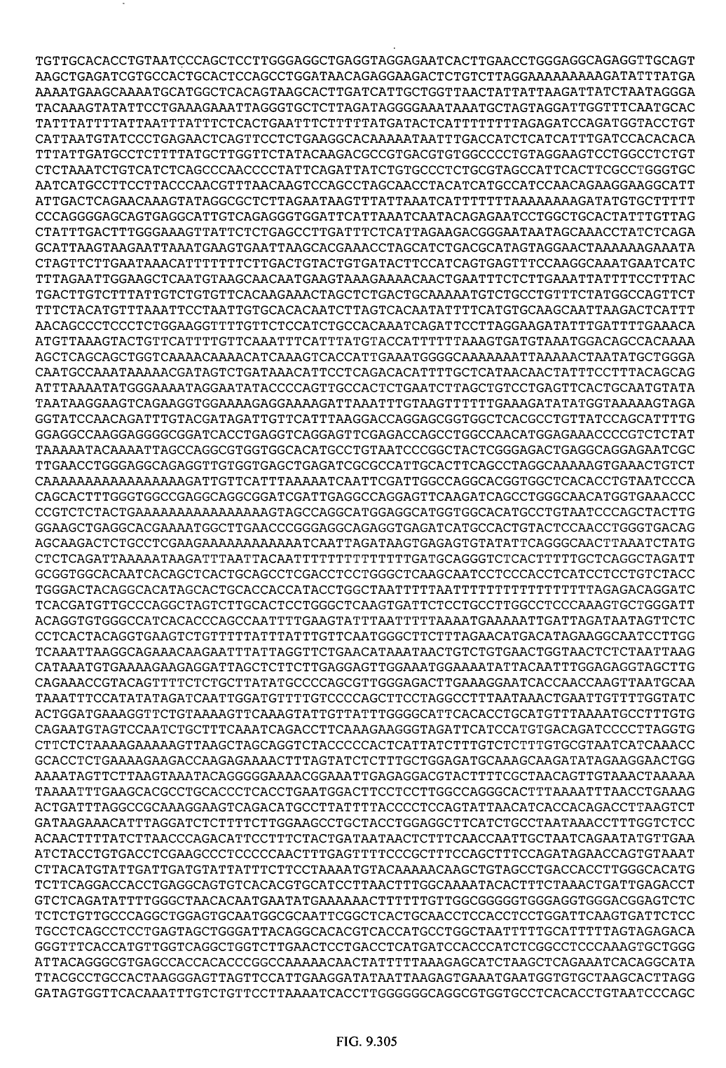

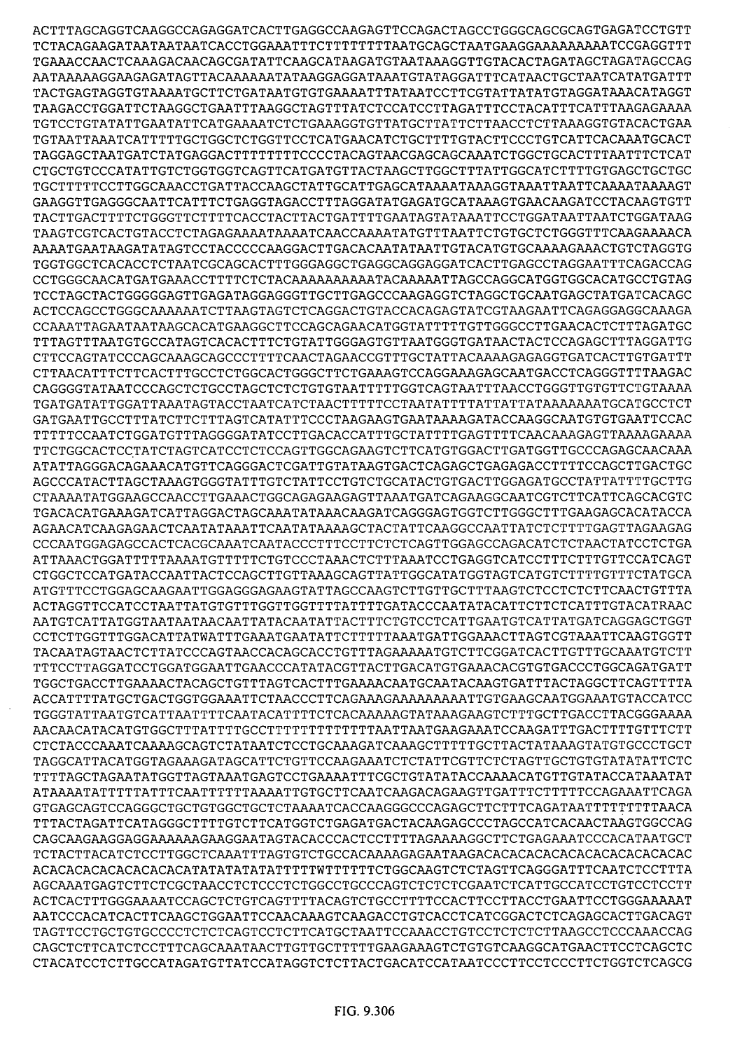

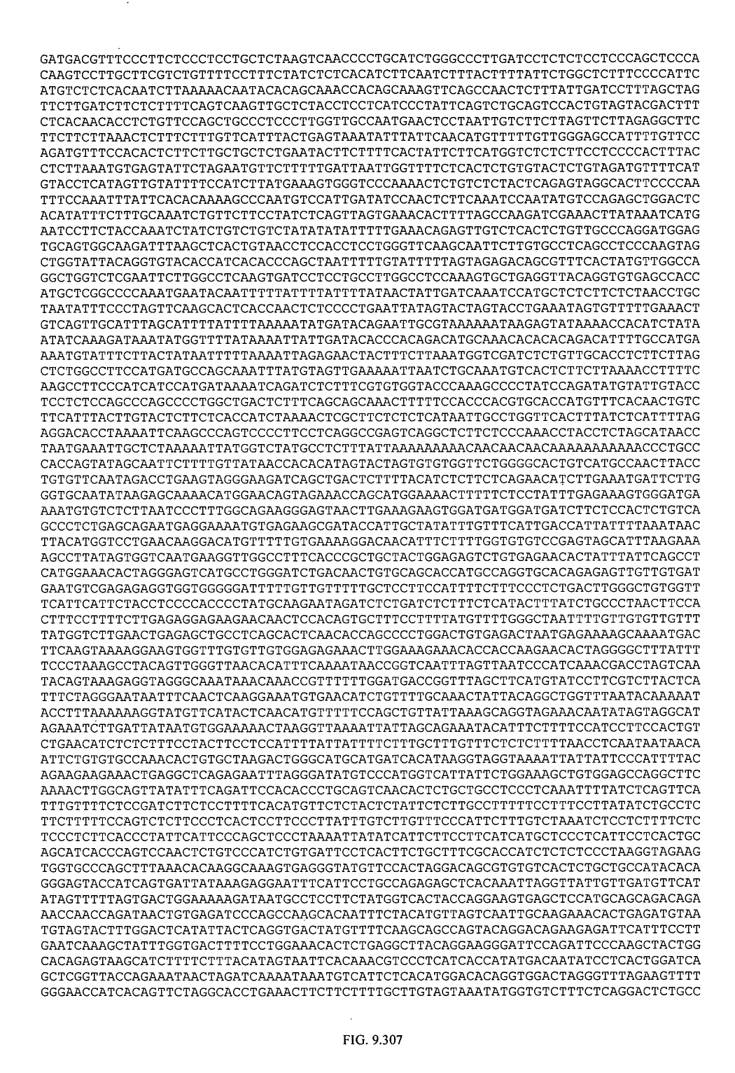

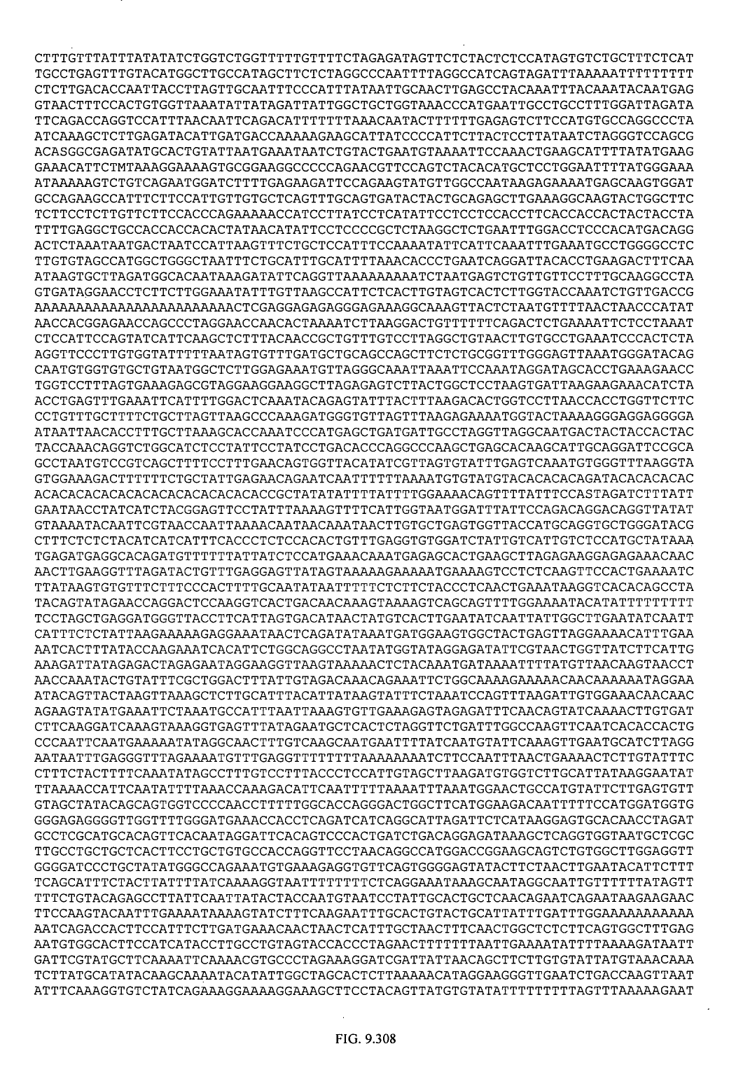

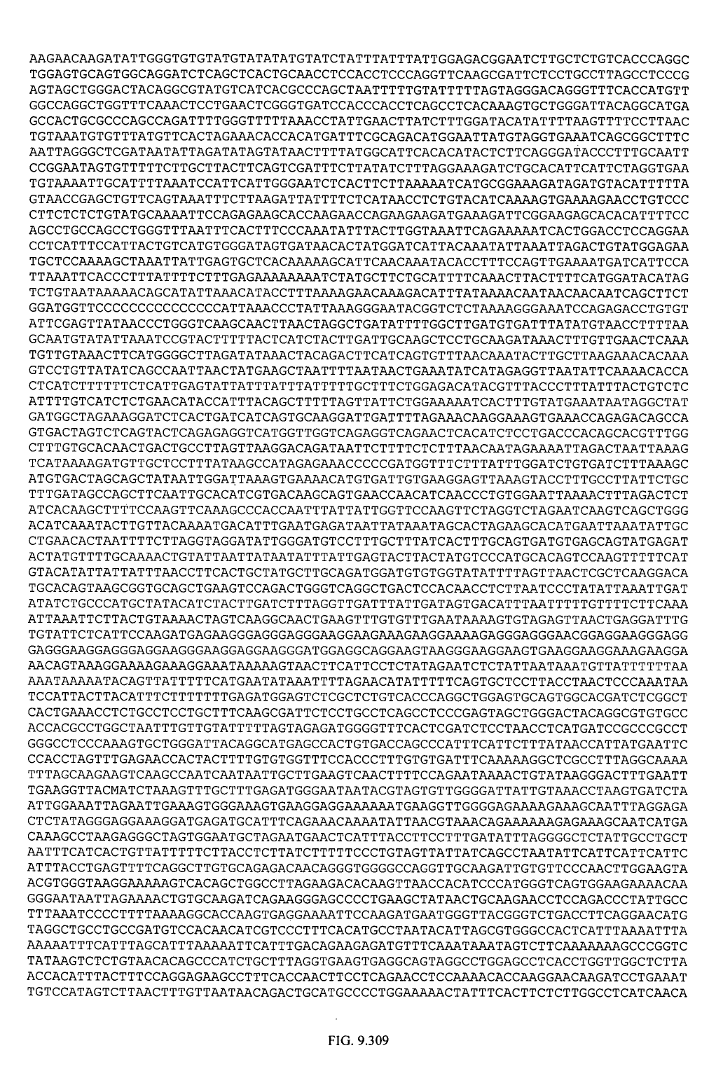

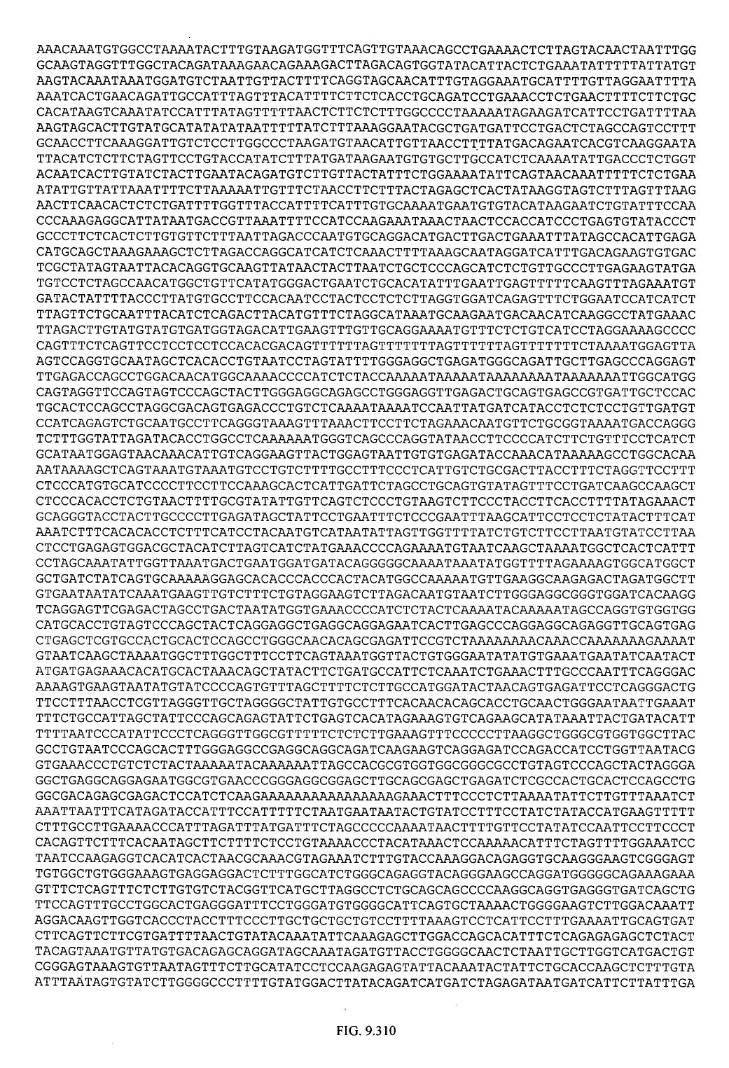

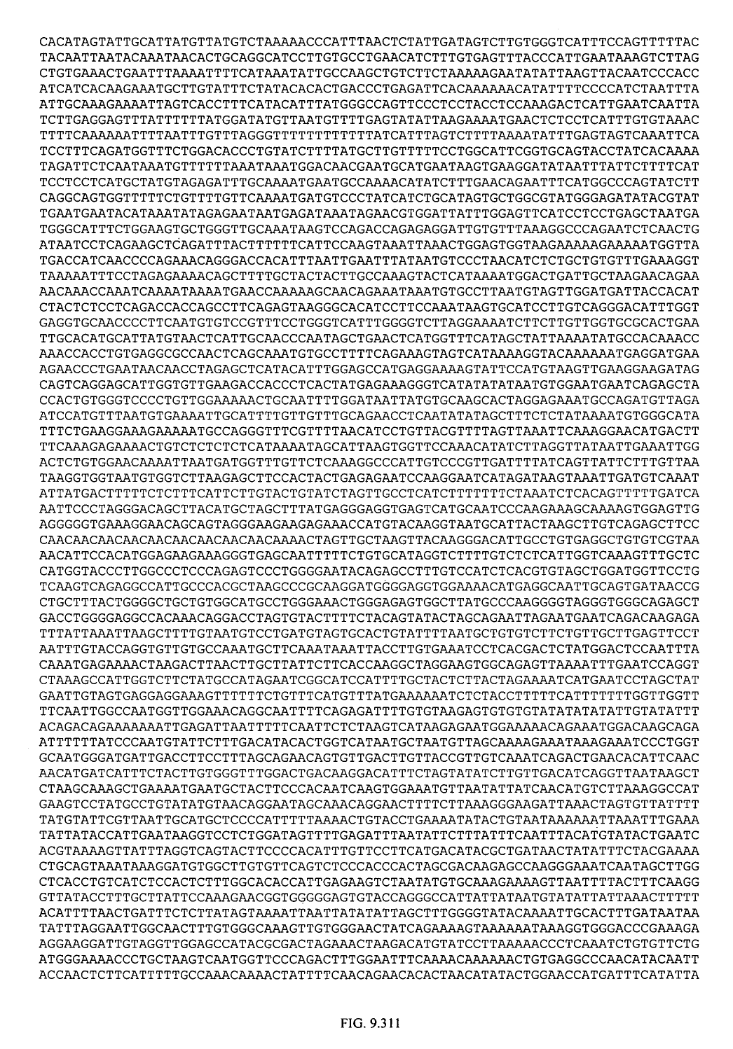

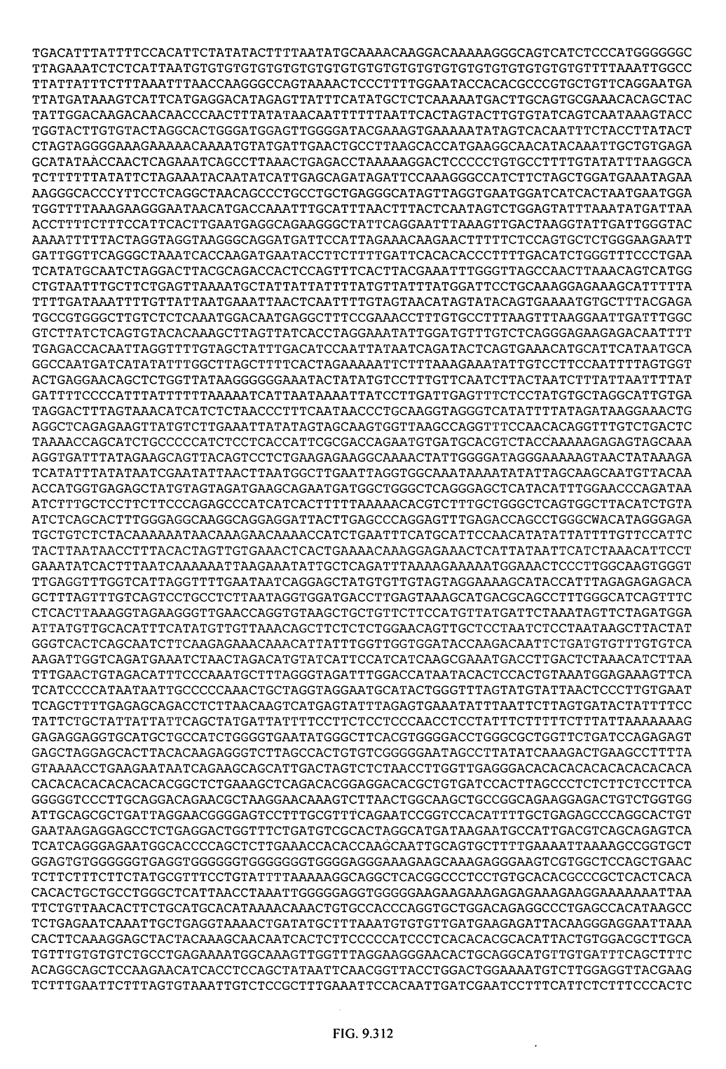

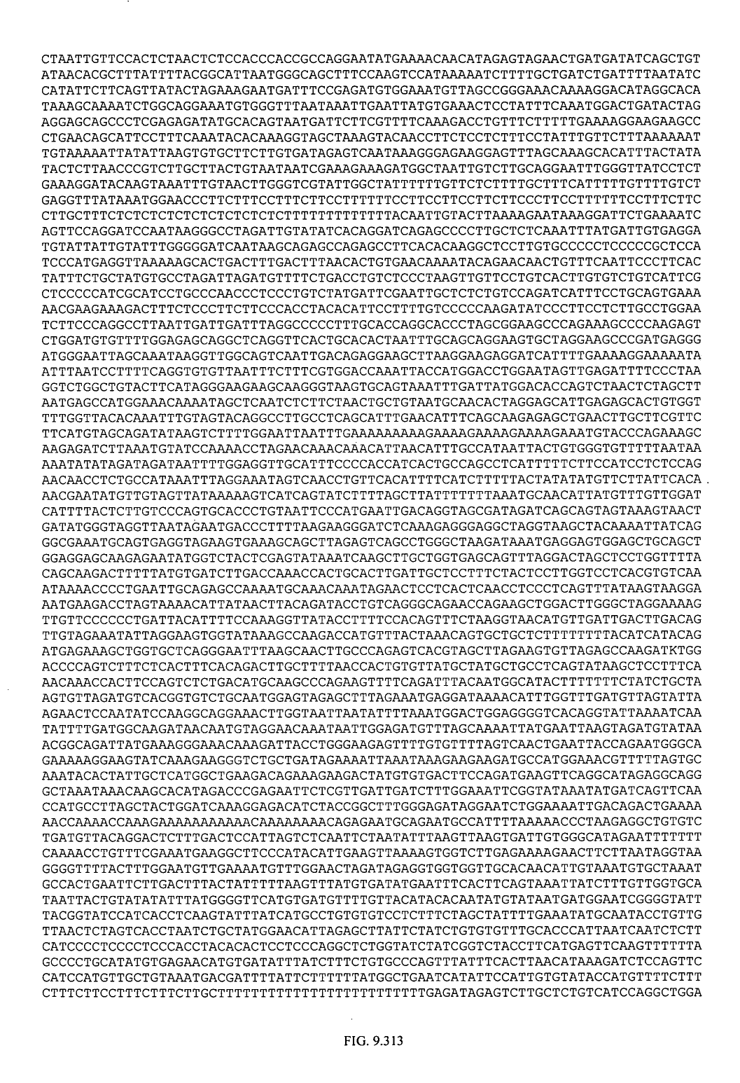

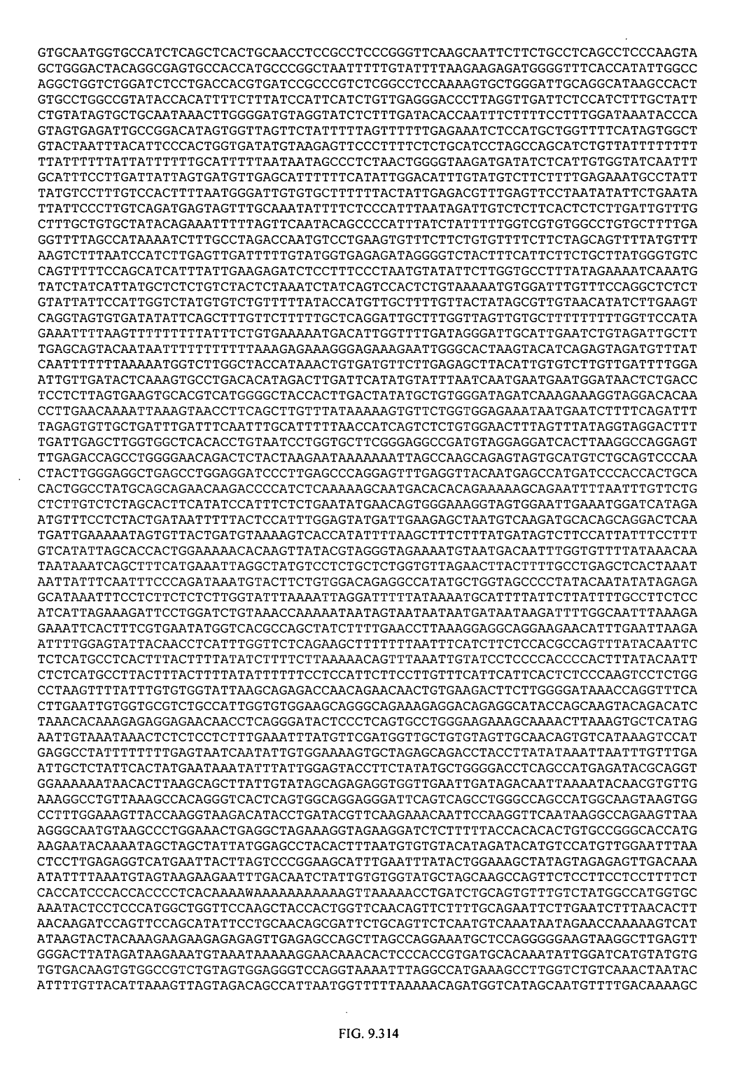

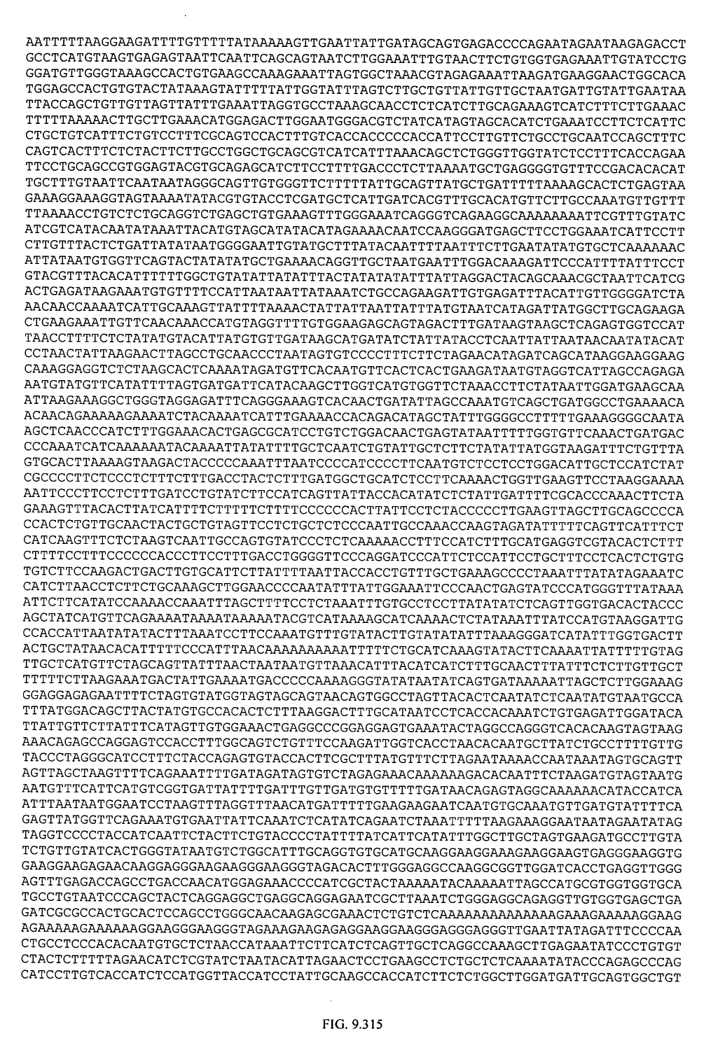

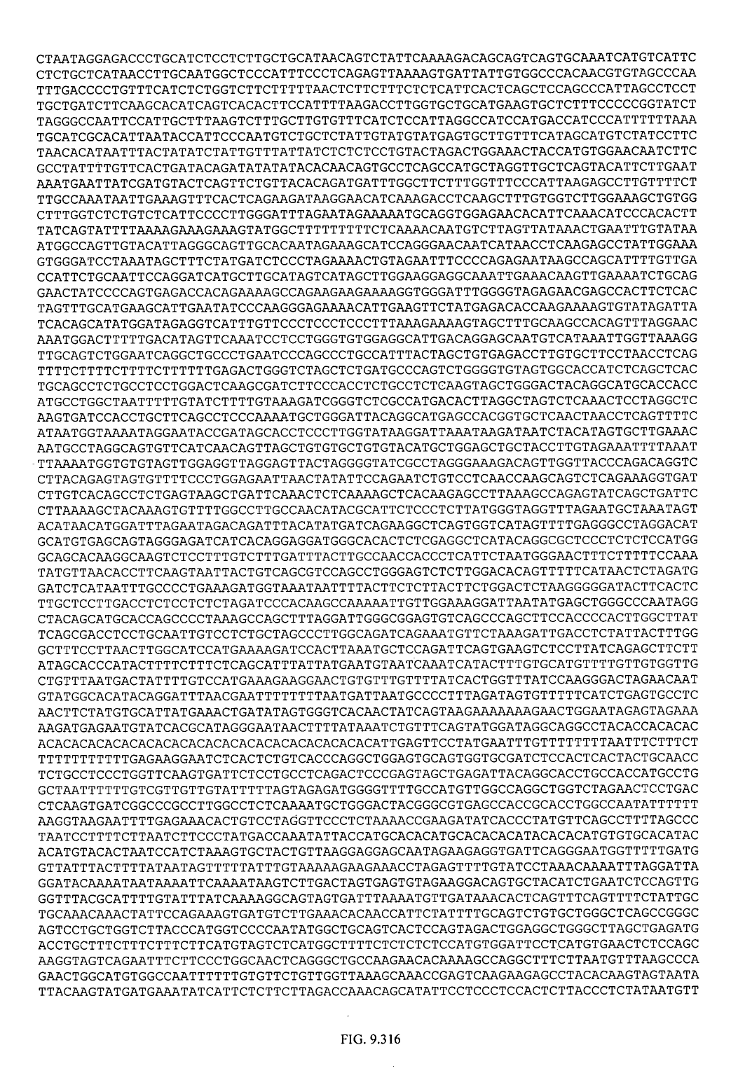

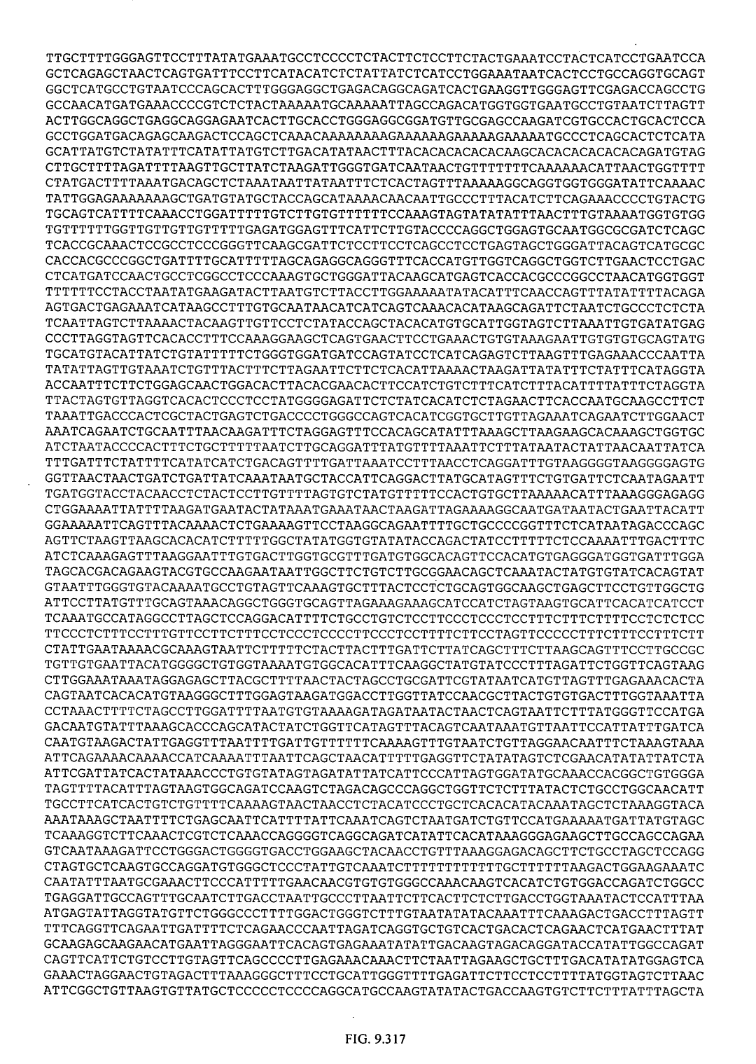

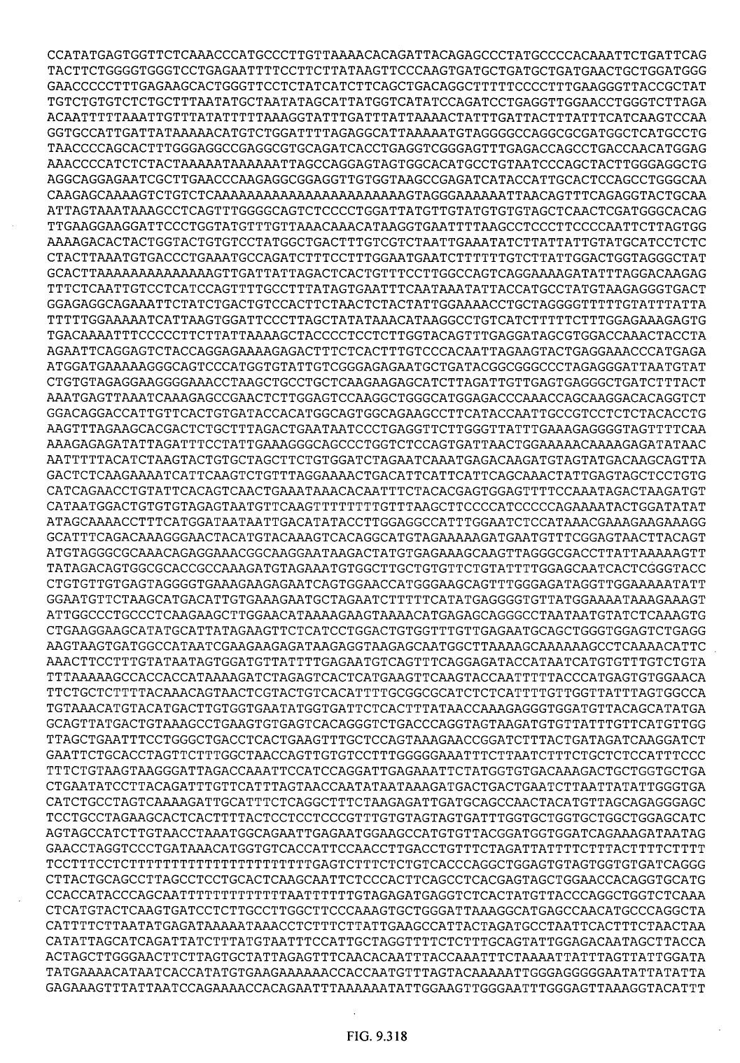

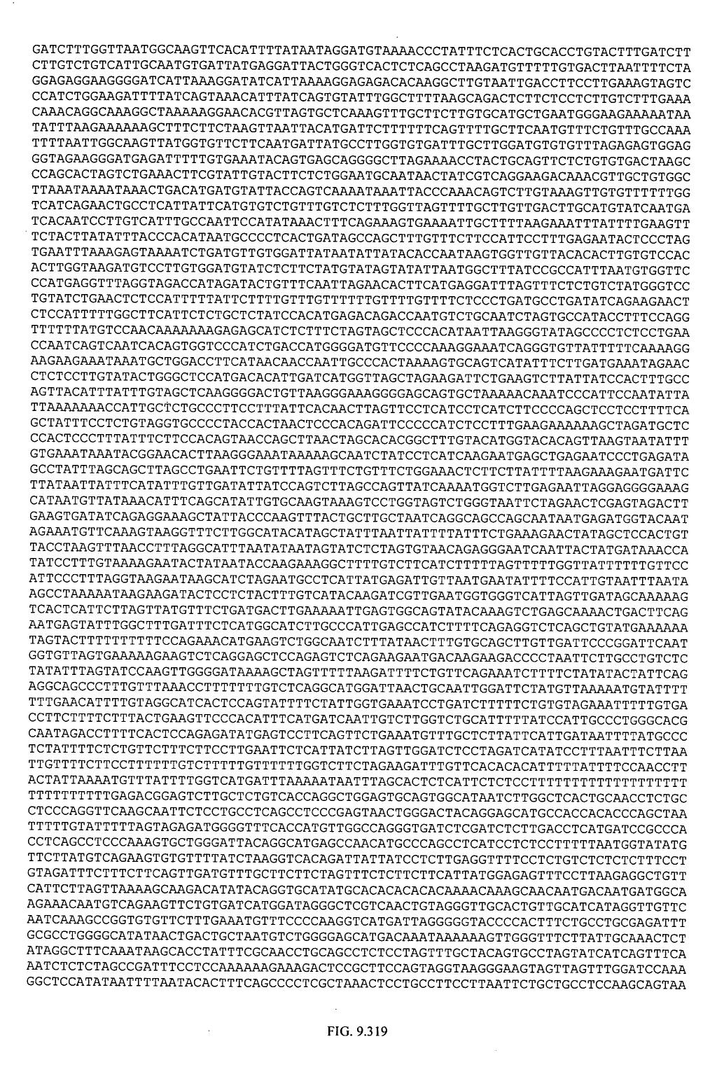

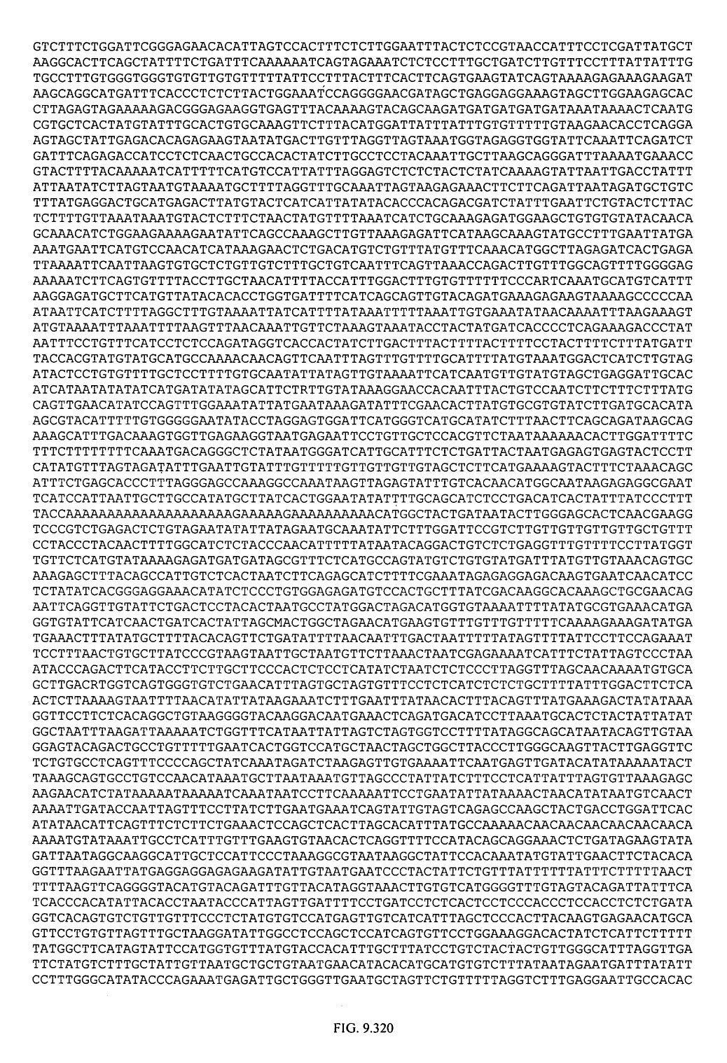

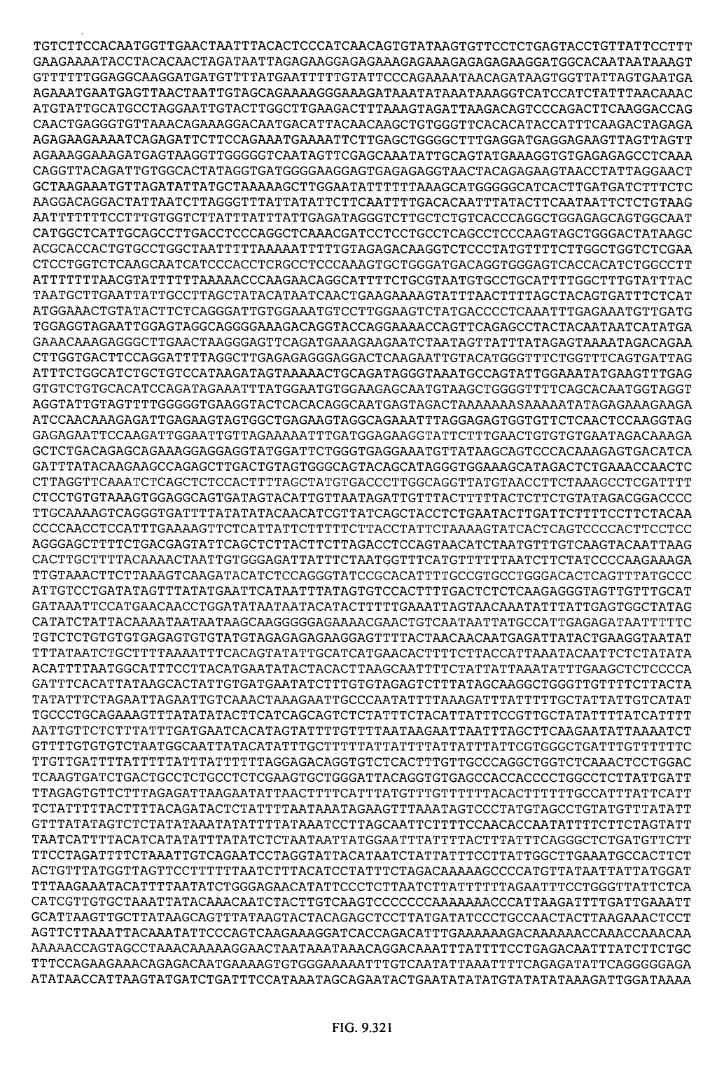

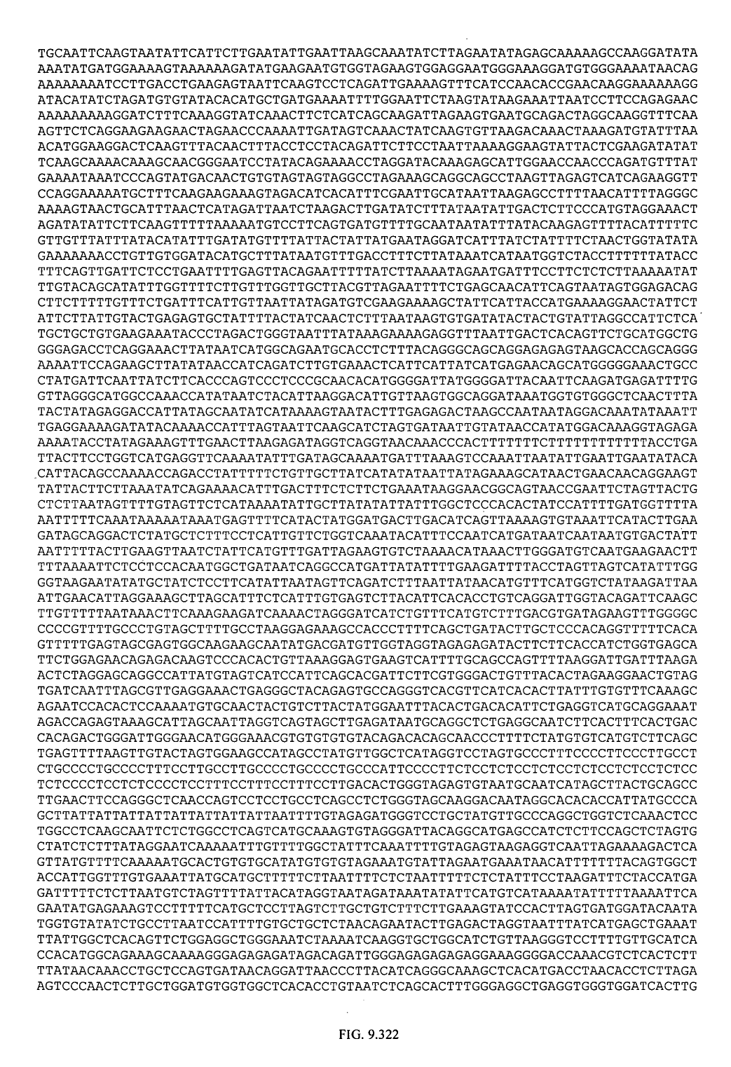

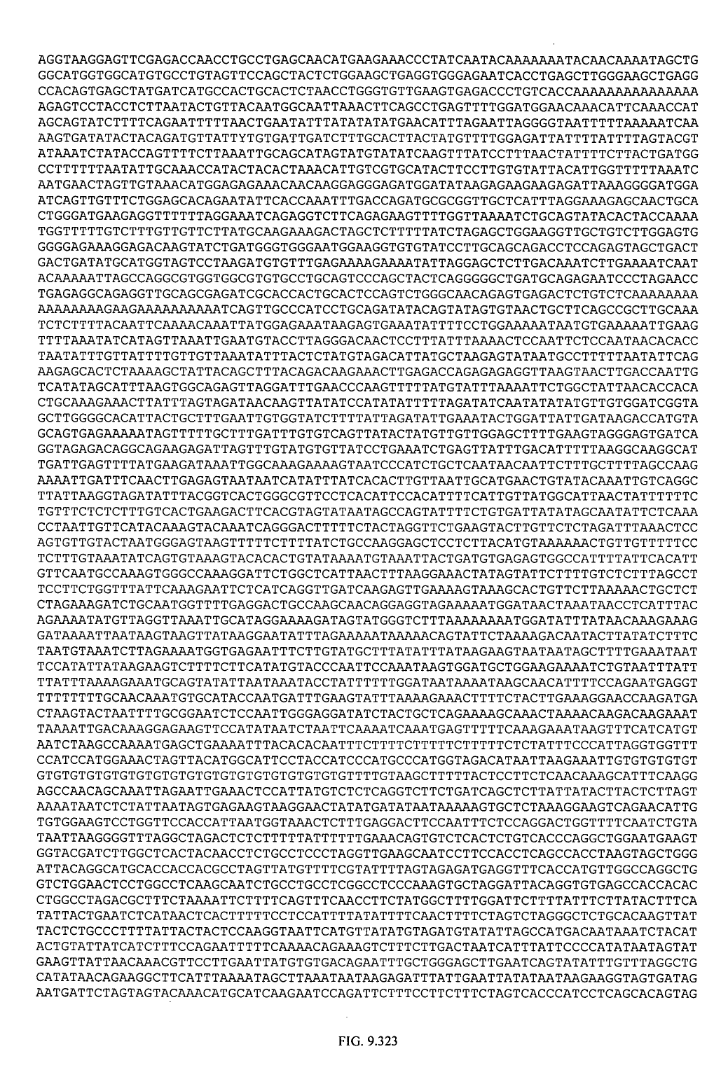

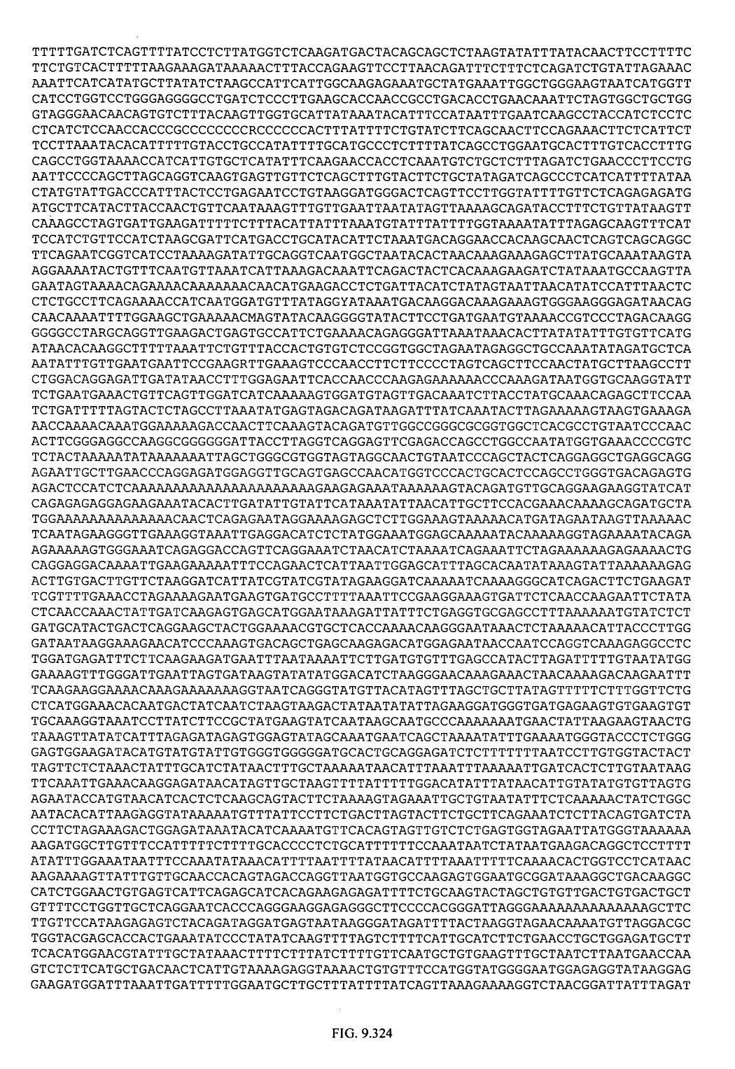

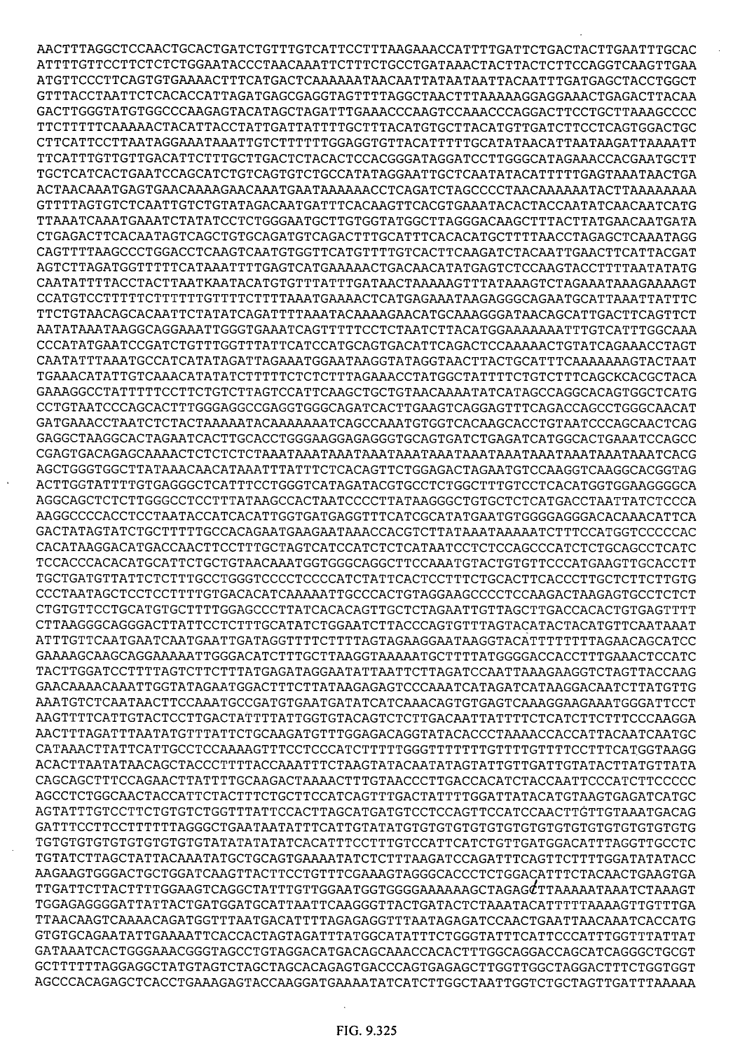

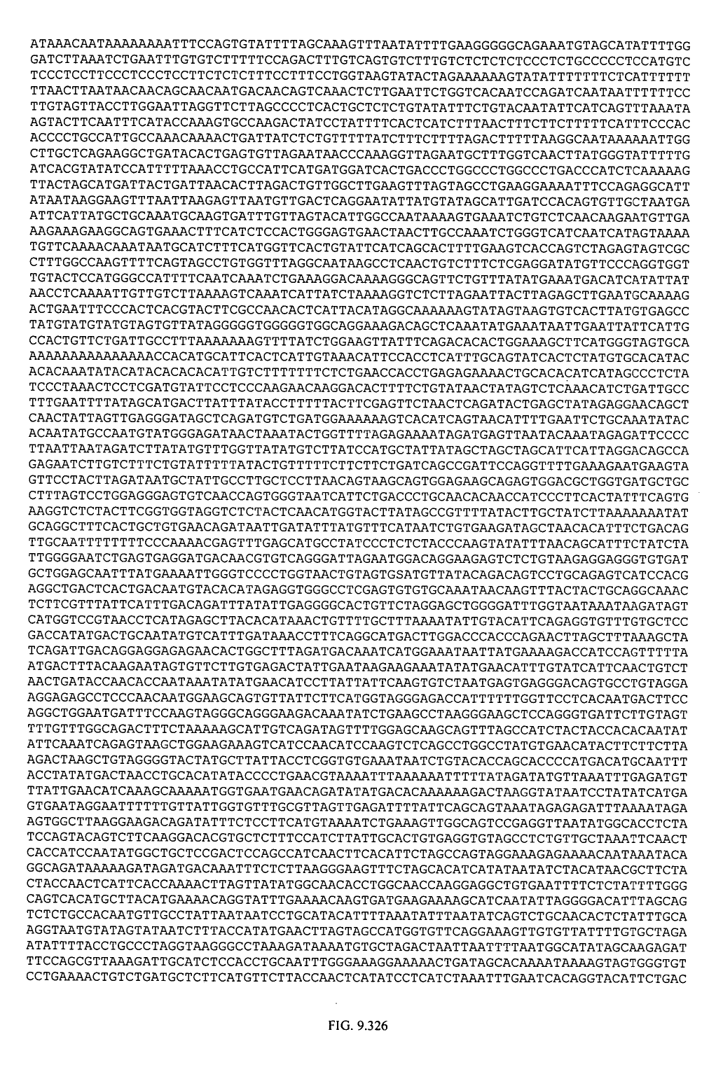

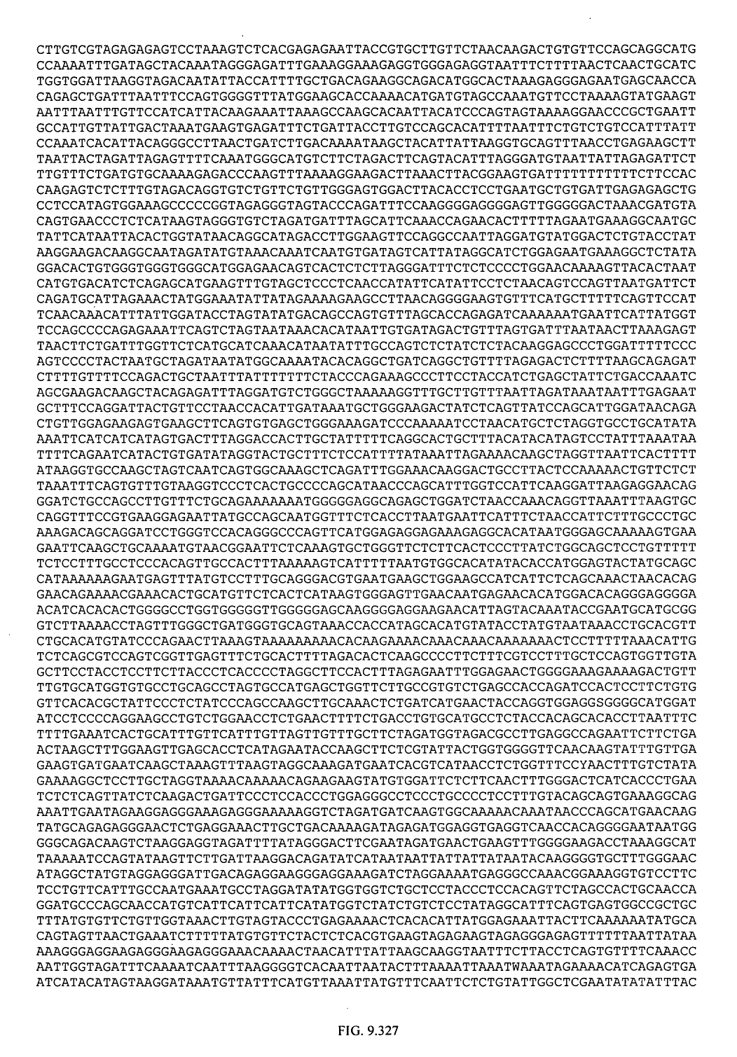

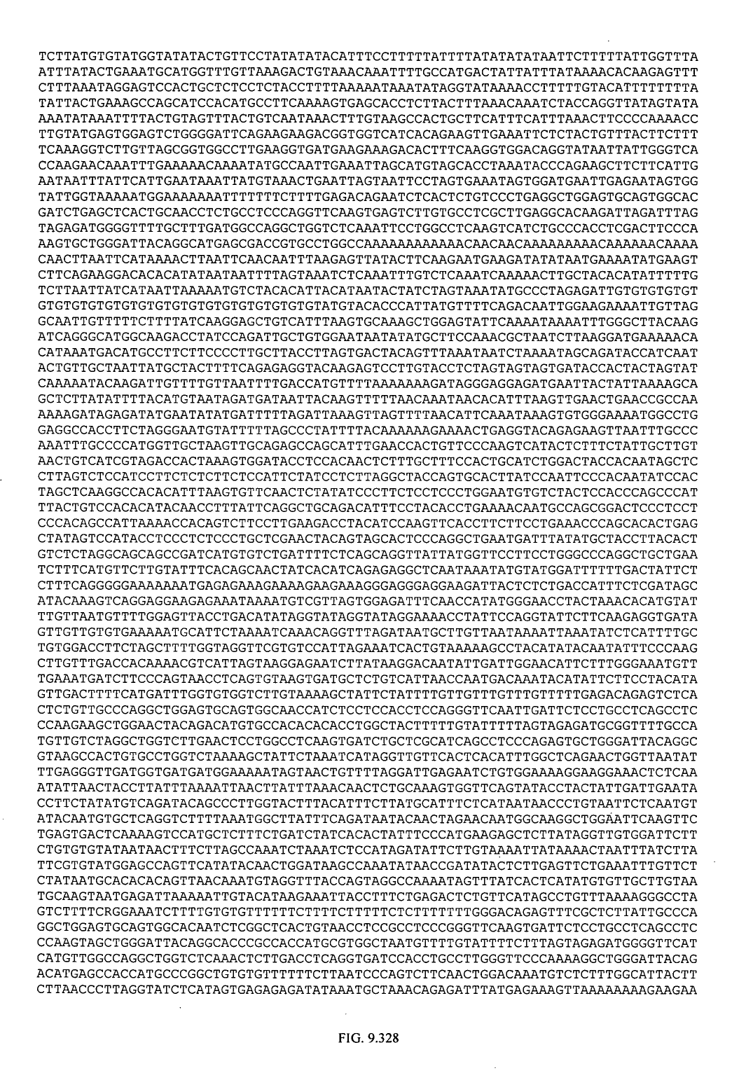

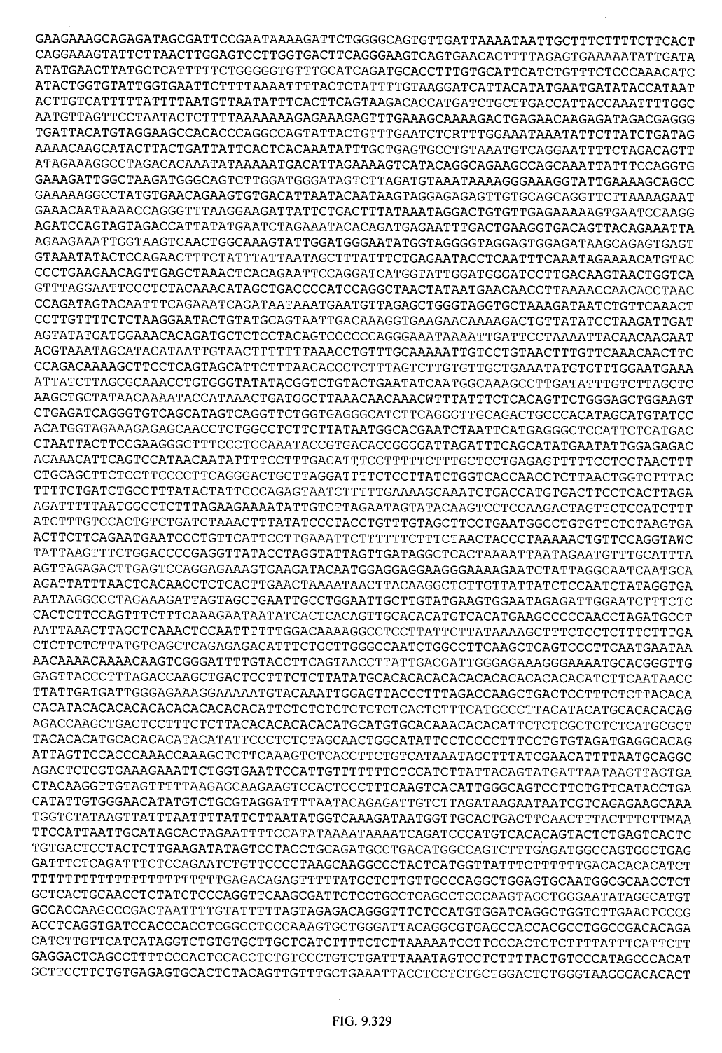

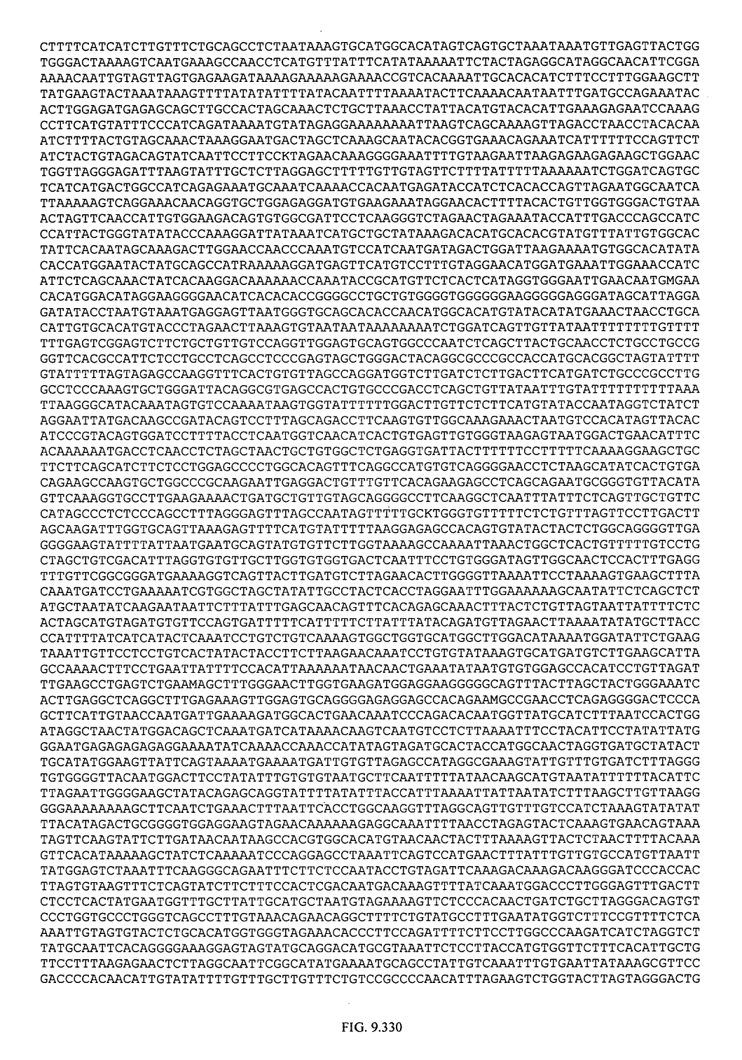

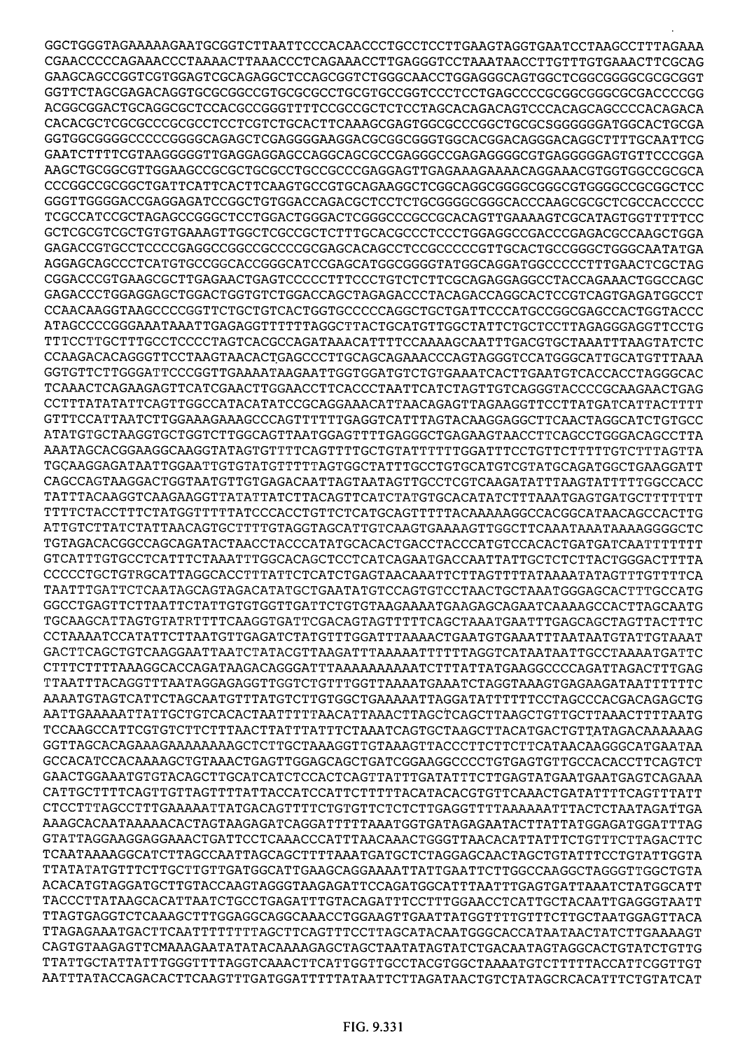

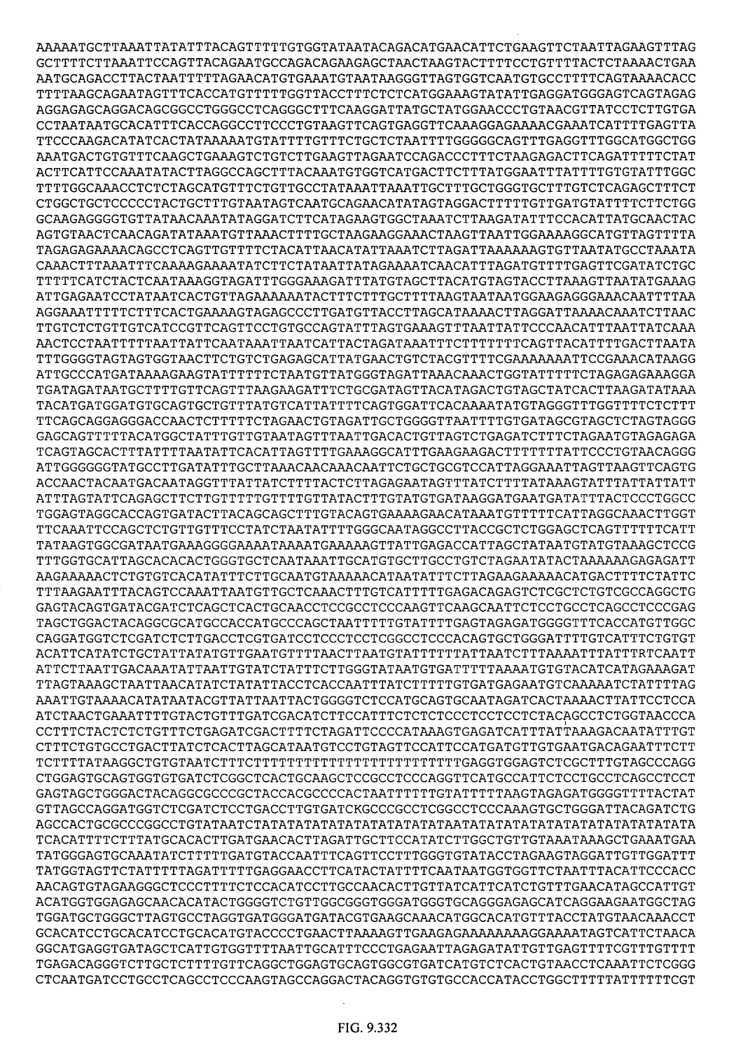

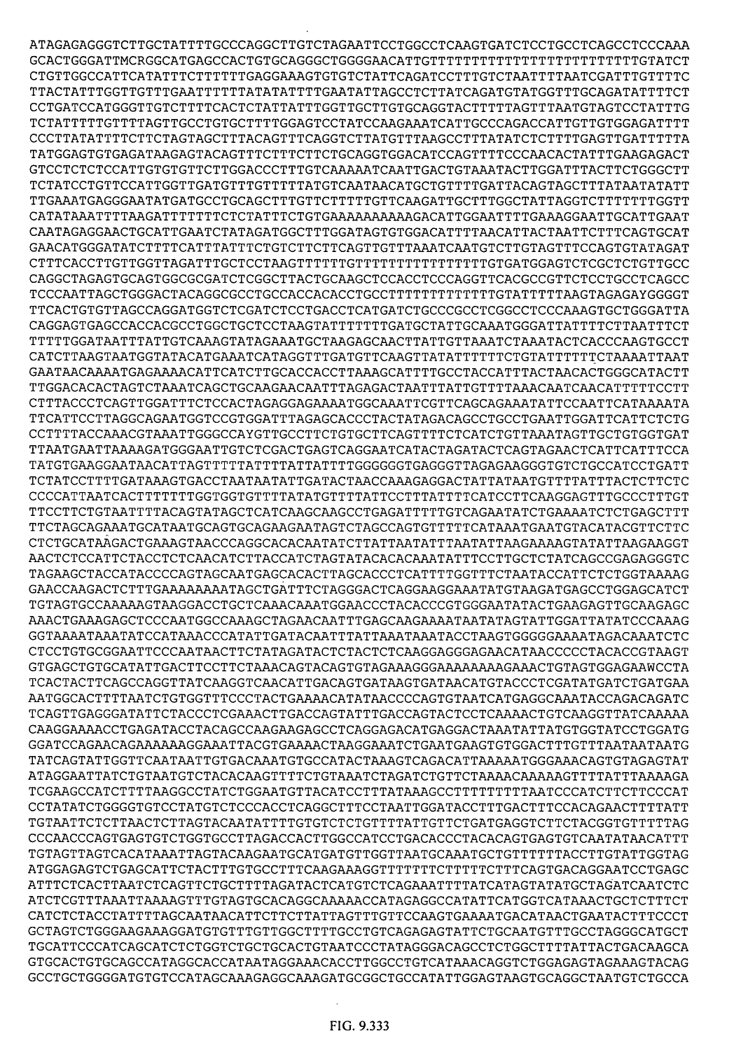

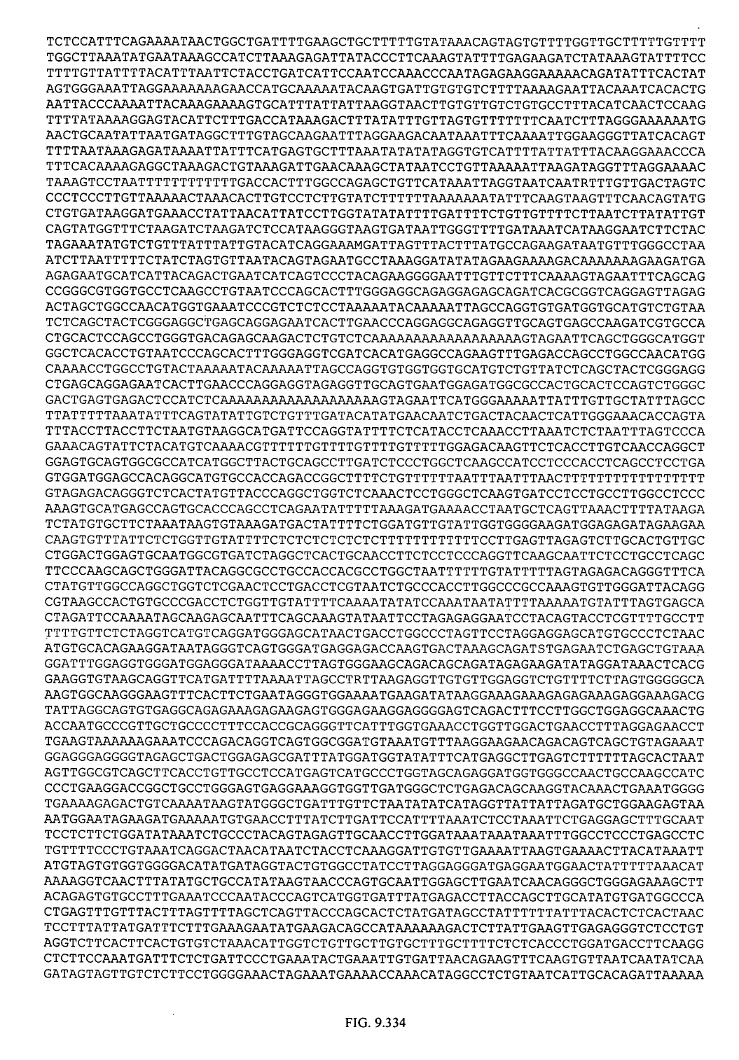

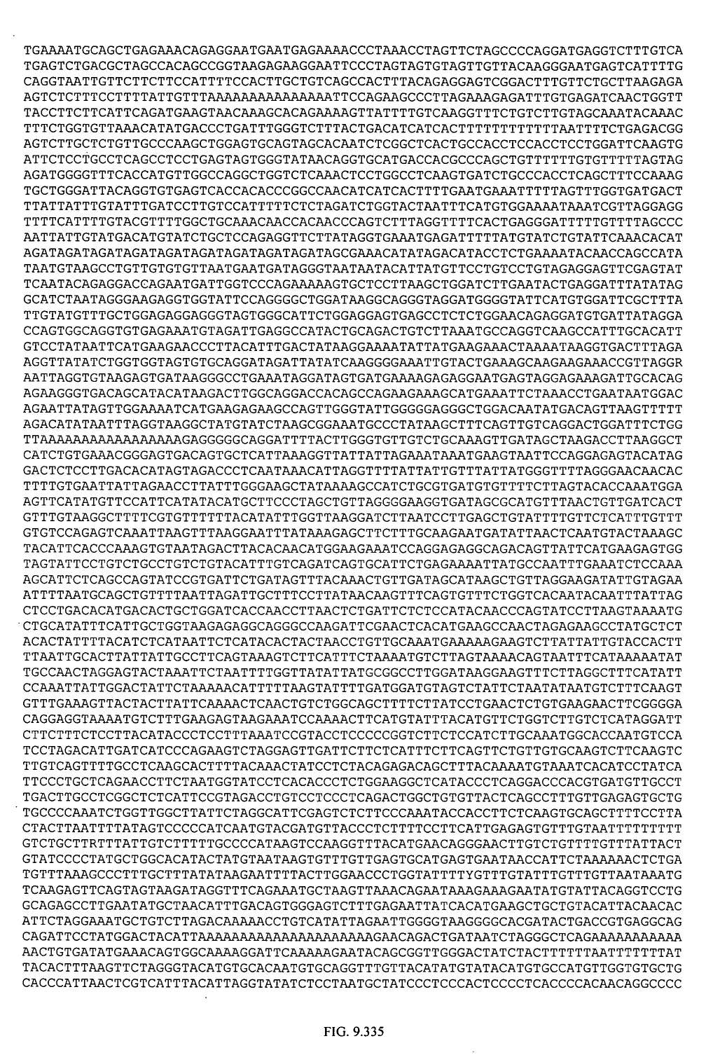

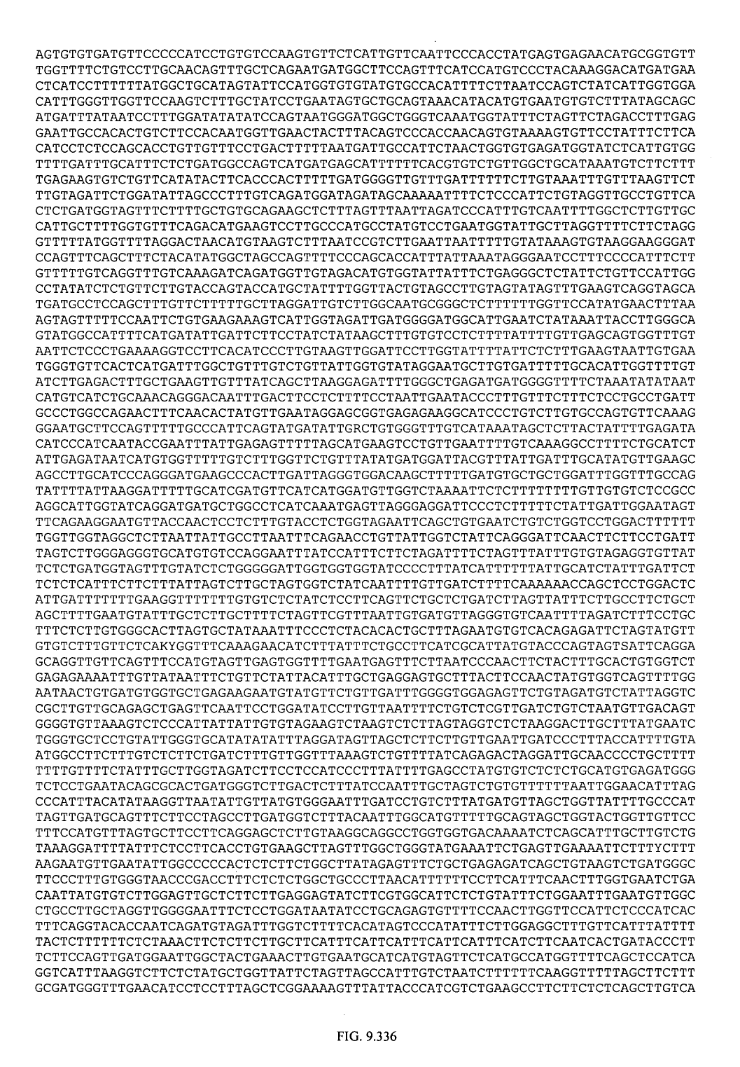

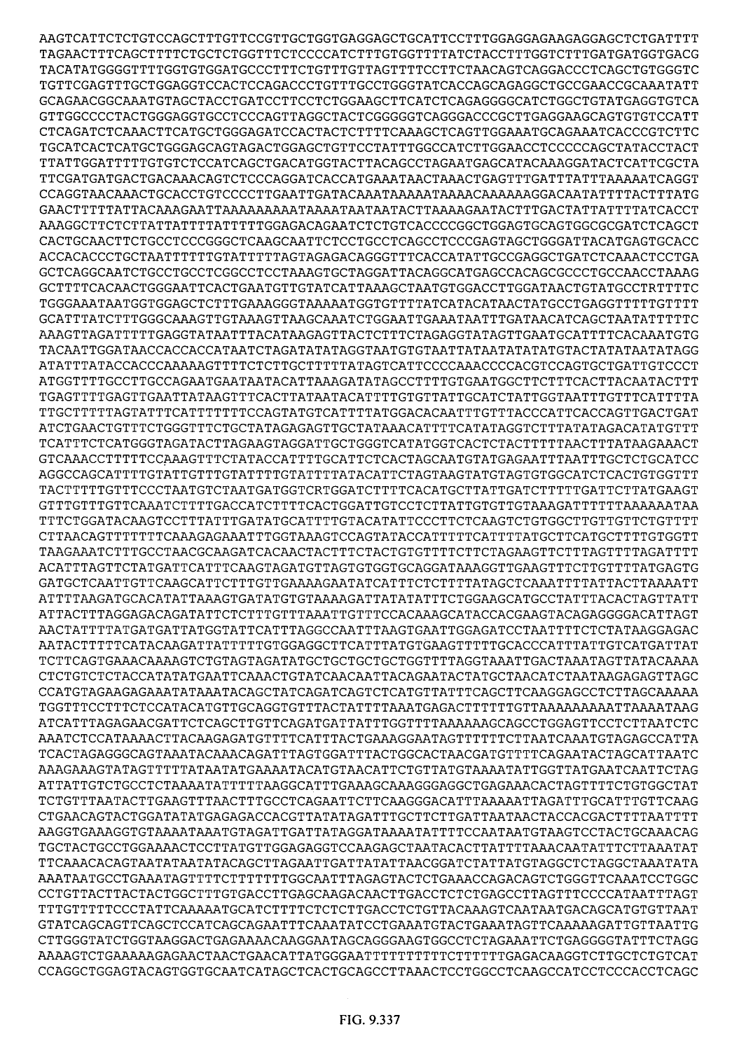

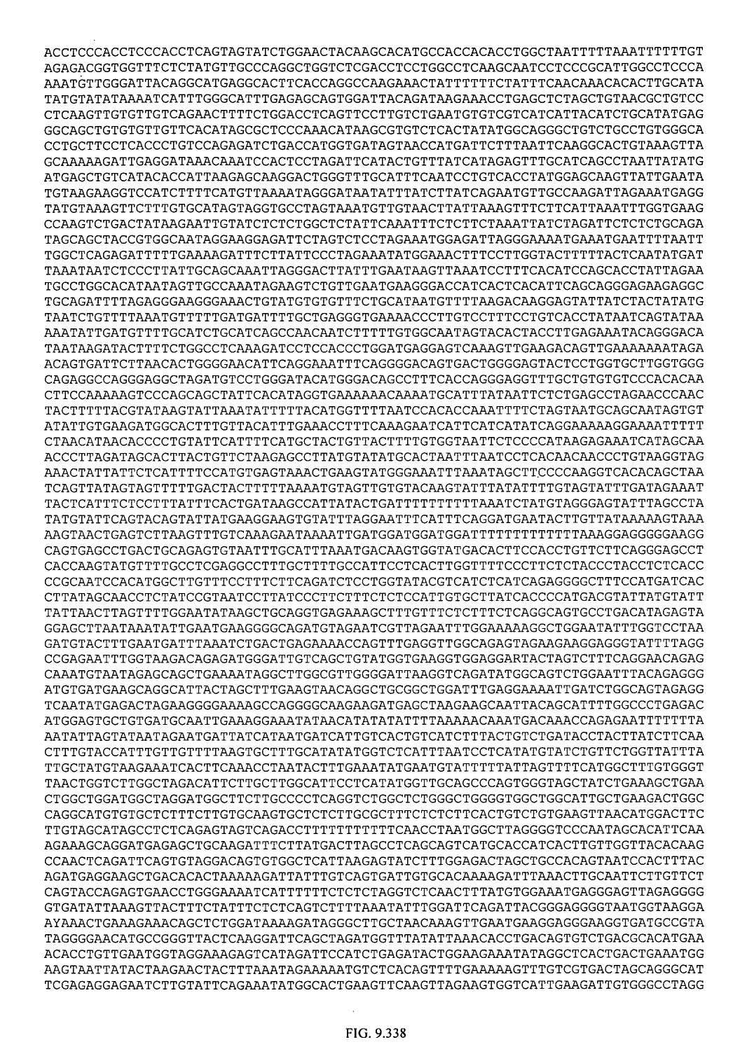

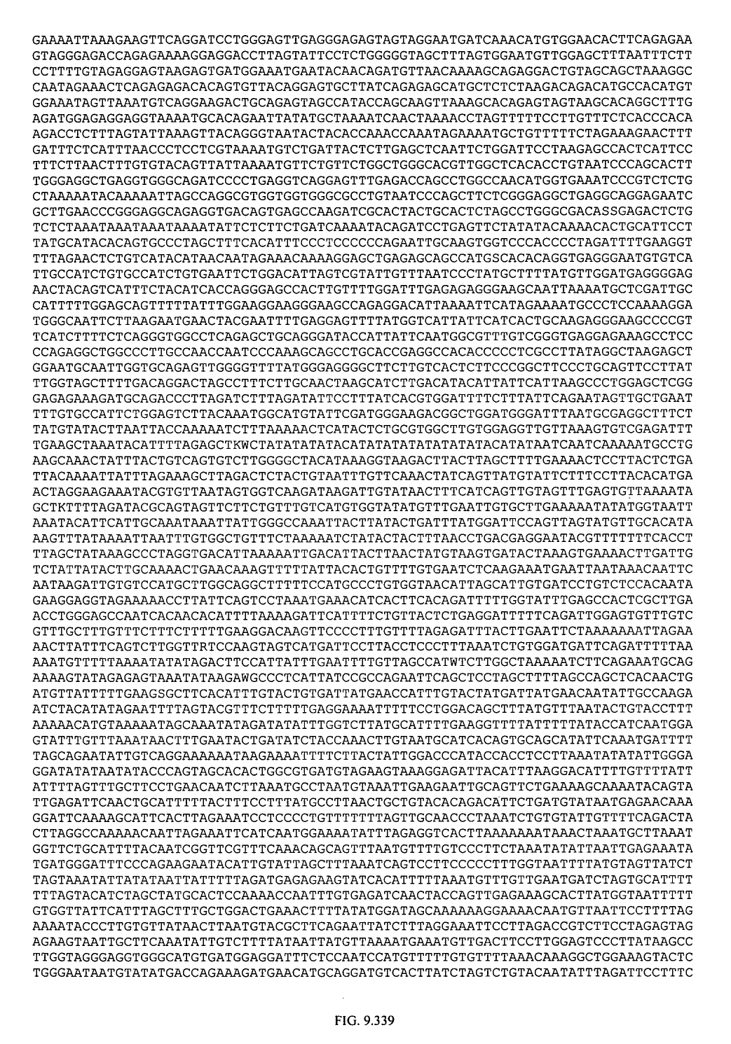

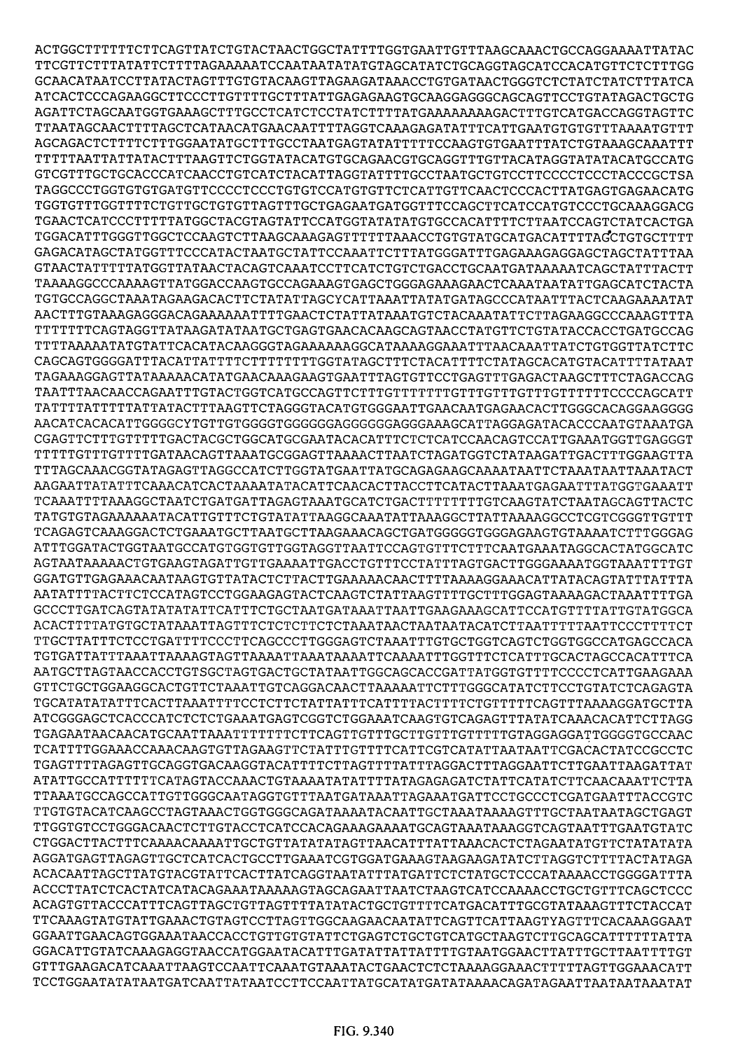

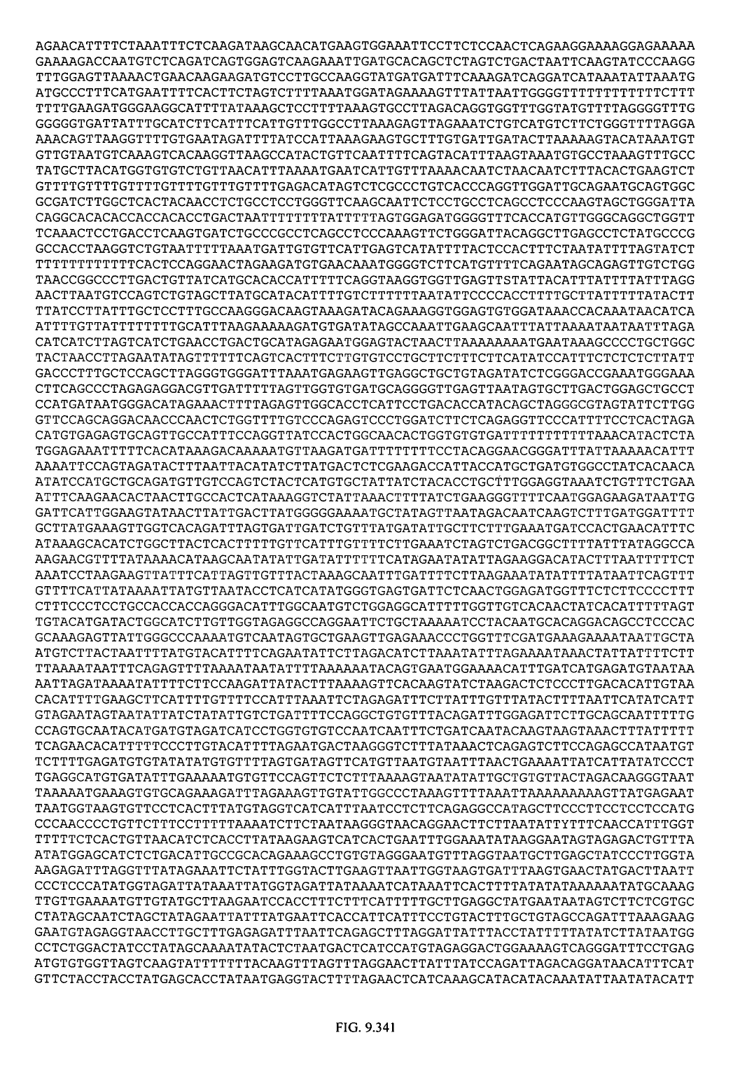

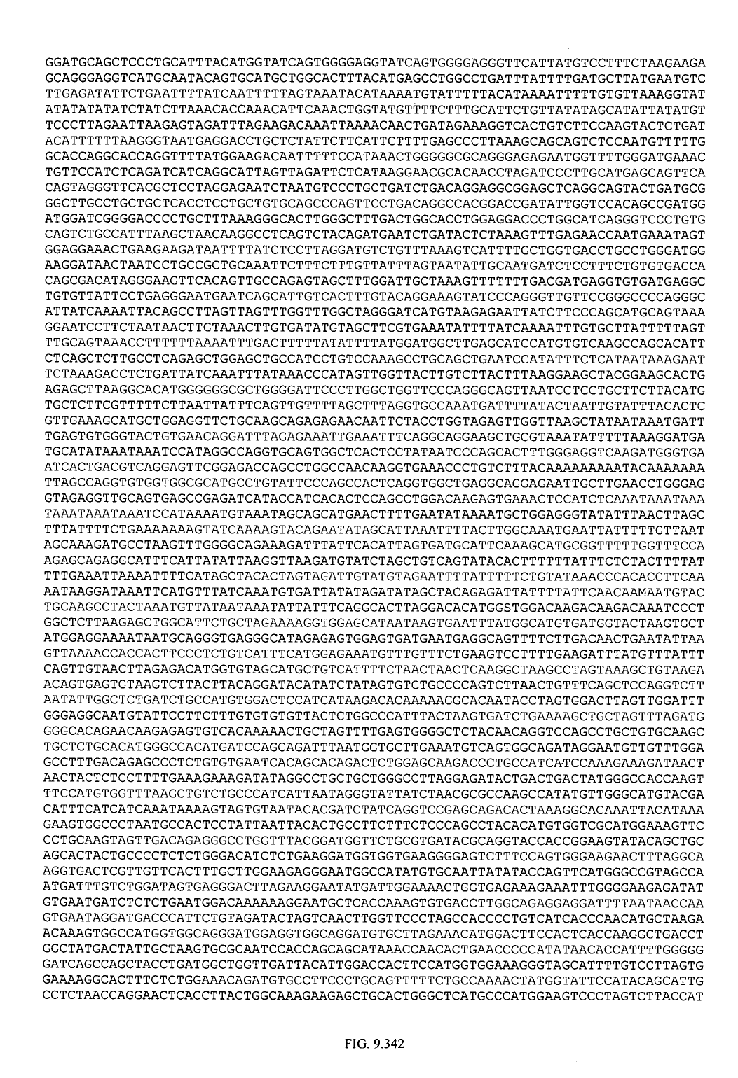

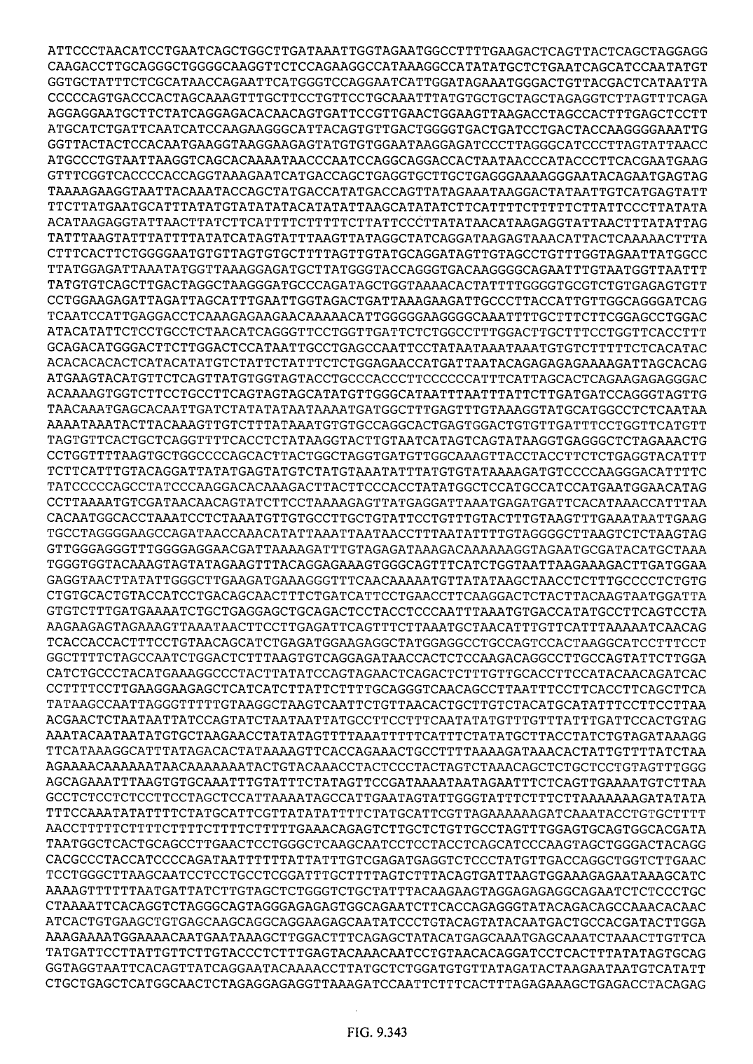

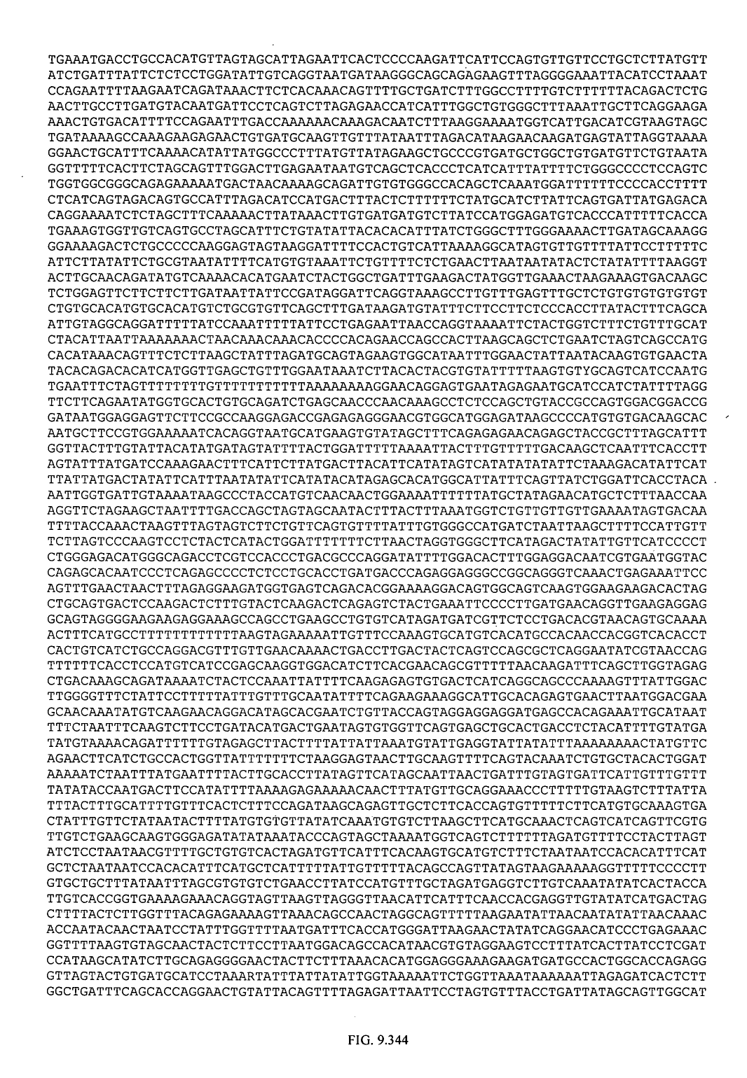

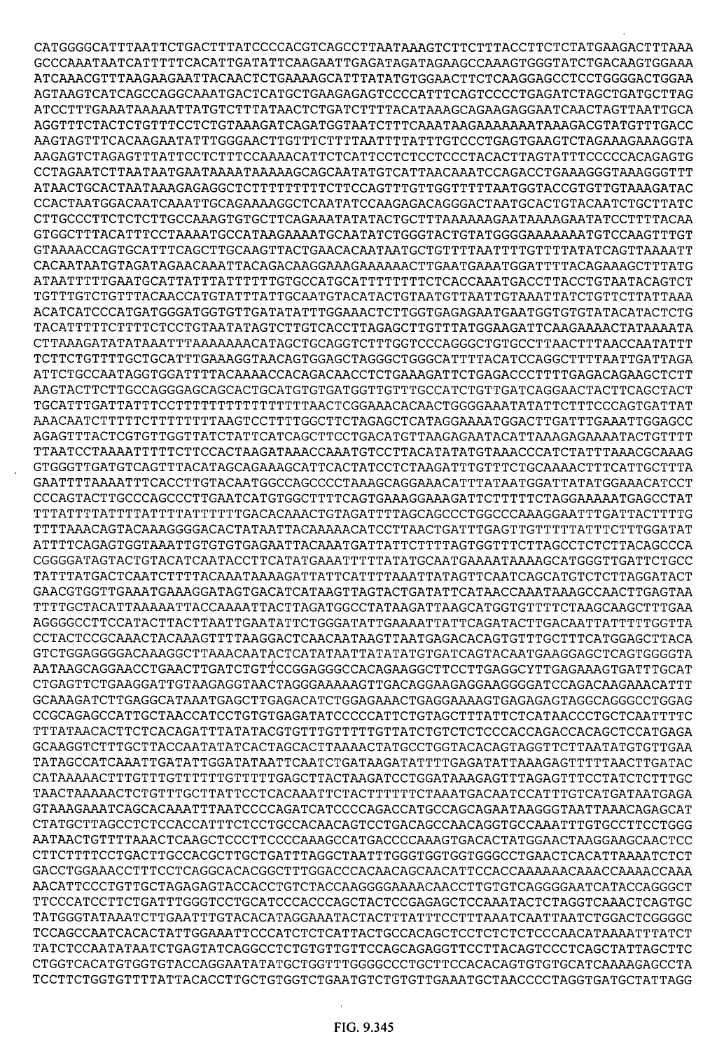

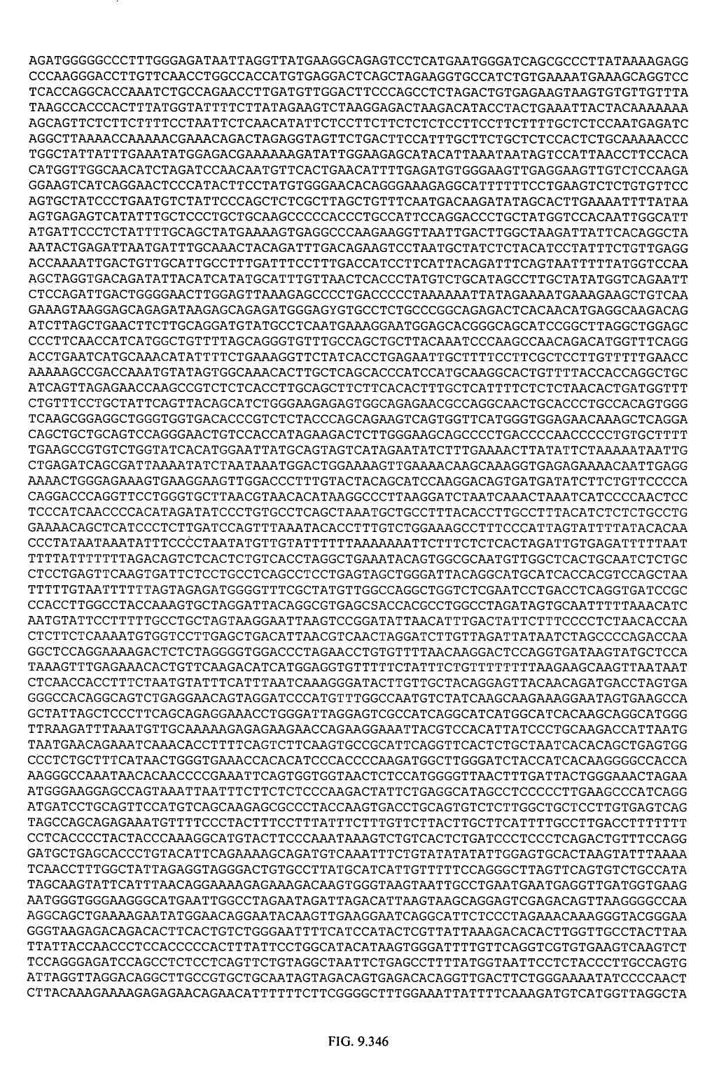

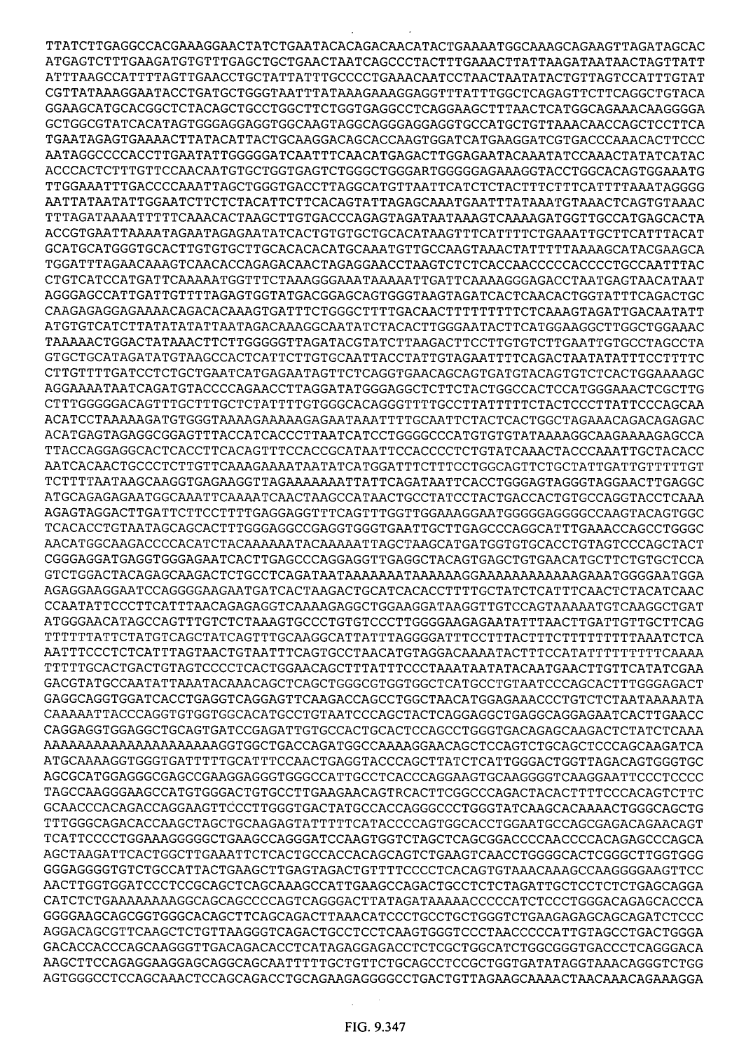

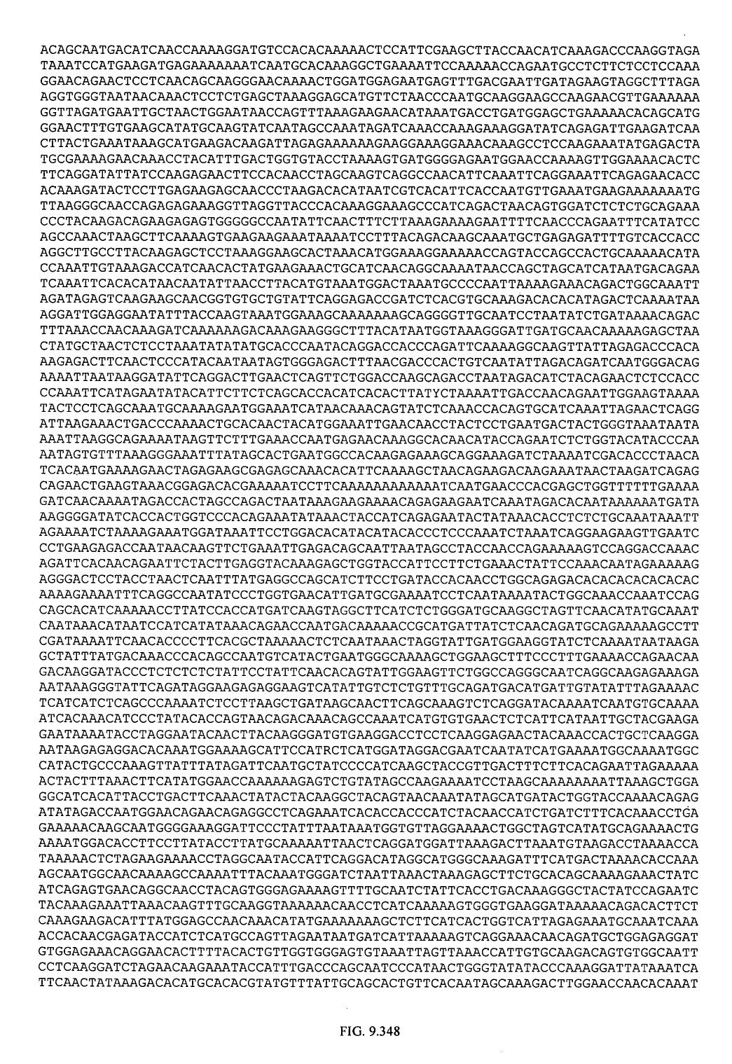

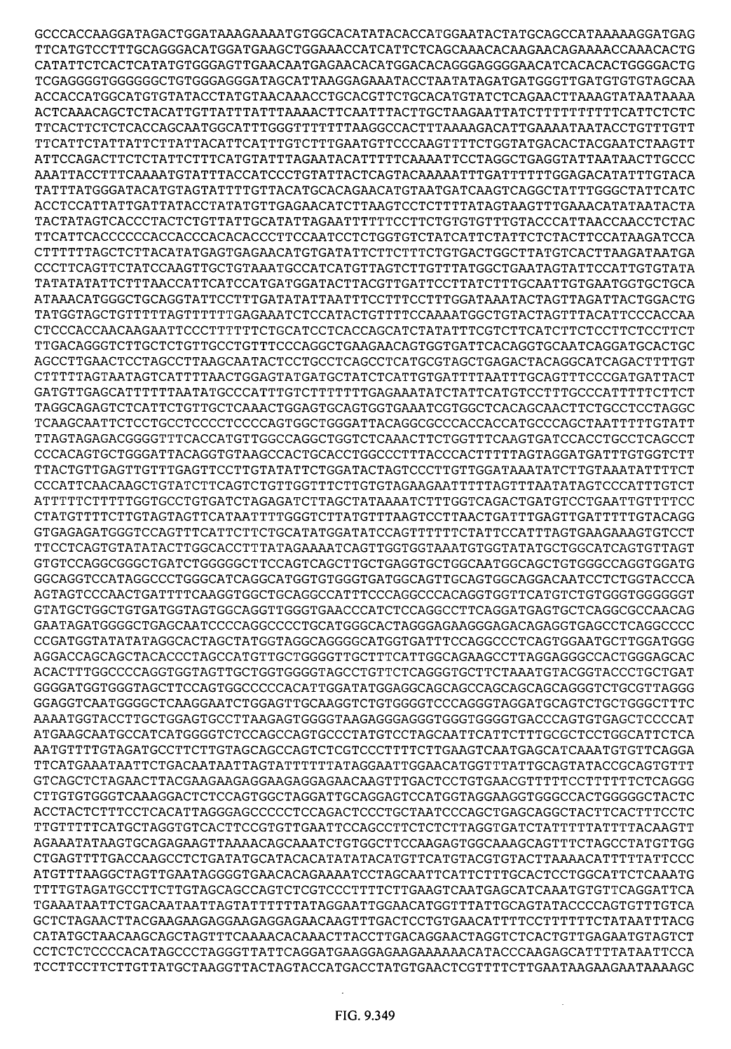

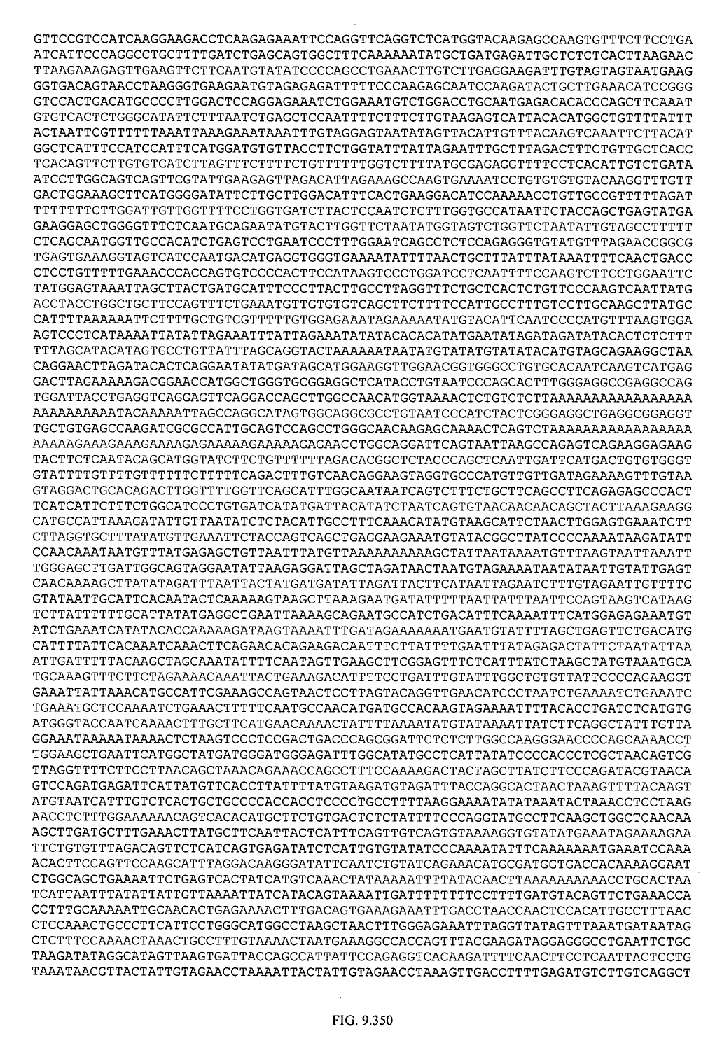

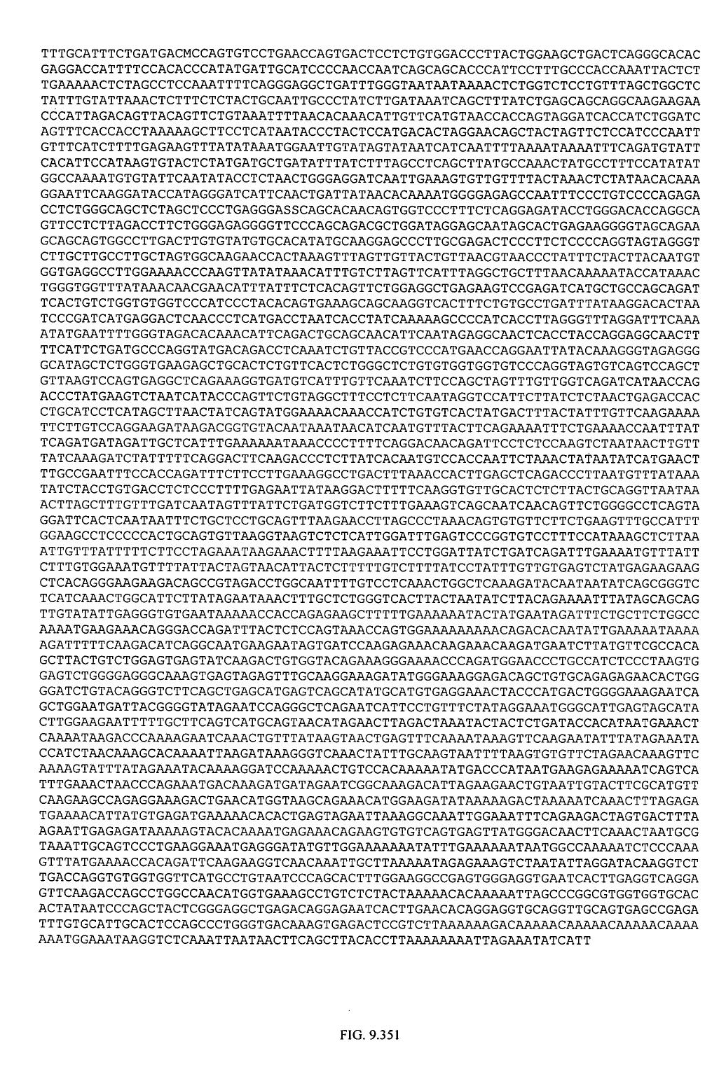

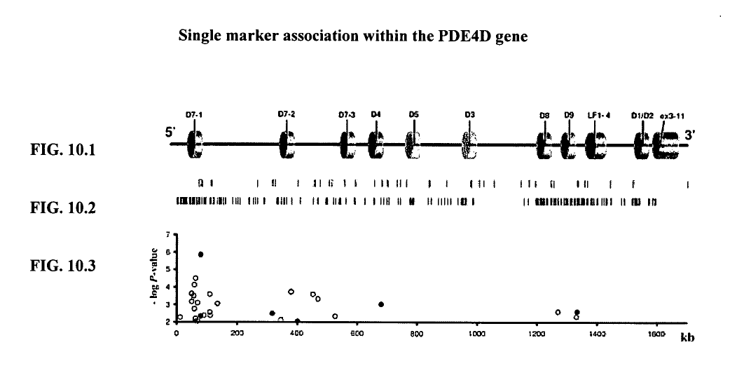

We selected individuals with a specific stroke associated haplotype and compared the expression levels of carrier vs. non-carriers of this haplotype and with patients and controls examined separately (FIGS. 5 and 6). The haplotype was constructed out of the at-risk allele for the microsatellite marker AC008818-1 and SNP45 (SNP5PDM357221) and SNP41 (SNP5PDM361545). This haplotype acts as a surrogate for the disease-associated haplotype we have identified in LD block B (Table 3). Patients with the haplotype had a significantly decreased expression of the PDE4D7 and PDE4D9 isoforms (FIG. 5). Several other isoforms of PDE4D were expressed but did not show correlation to the disease haplotype. The PDE4D7 correlation was also present in controls but only marginally significant (FIG. 6). Of interest, this at-risk haplotype covers the 5′ exon specific to PDE4D7 and presumably its promoter.

These results show that there is significant disregulation of the expression of multiple PDE4D isoforms in stroke patients.

Methodology for Expression Analysis Using Quantitative Reverse Transcriptase PCR

Total RNA was isolated from EBV transformed B-cell cultures according to manual, using the TRIZOL® reagent provided by GibcoBRL. RNeasy mini Qiagen kit with on column DNA digestion was used to clean RNA. Quality and quantity of RNA was assessed using 2100 Agilent Bioanalyser. cDNA was prepared from total RNA using random hexamers with TaqMan Reverse Transcription Reagents kit from Applied Biosystems (N808-0234). Primer Express 2.0 and Oligo 6 software were used to make cDNA specific primers and probes for PDE4D and PDE4D isoforms. GAPDH “Assay-On-Demand” was obtained from Applied Biosystems and used as a housekeeping gene. PDE assays were tested and optimized for 384 well high throughput expression analysis using ABI 7900 Instrument. A final concentration of 200 nM probes, 900 nM primers and 2 ng/mcl cDNA was used in a 10 mcl reaction volume. Each plate was run twice and an average for each sample calculated. ABI7900 instrument was used to calculate CT (Threshold Cycle) values. Samples displaying a greater than 1 deltaCT between duplicates were not used in our analysis. Quantity was obtained using the formula 2^{-ΔCT} where ΔCT represents the difference of CT values between target and housekeeping assay.

Accession numbers AY245866 (PDE4D7) and AY245867 (PDE4D9).

Discussion of the Three Examples and Conclusions:

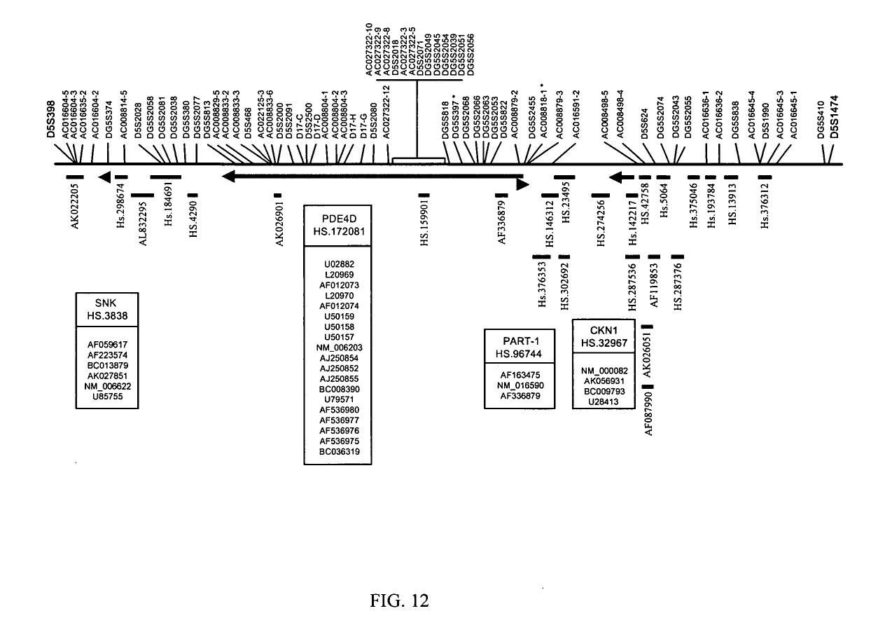

Our results indicate that genetic variation in the PDE4D gene is associated with ischemic stroke. The direct involvement of PDE4D is strongly supported by linkage in conjunction with association and expression analysis. We first identified the association using microsatellite markers, and supplementing the microsatellite data with a denser set of SNPs further supported this. The strongest association is to the two ischemic subtypes, carotid and cardiogenic stroke whereas we did not observe association to small vessel occlusive disease, the form of stroke thought to be independent of atherosclerosis. Although we have not identified a functional mutation in the PDE4D gene, we have identified a haplotype, that extends over the first exon of PDE4D that is significantly associated to carotid and cardiogenic stroke. This haplotype is present in 47% of the carotid/cardiogenic stroke patients, compared to 21% in the control group with more than two-fold stroke risk for the carriers of this haplotype. It has a population attributed risk of 25%. For the combined cardiogenic and carotid subtype of stroke, apart from finding individual SNP and microsatellite alleles that are significantly associated with the disease even after adjusting for multiple comparison, most interesting is the discovery that haplotypes covering the first exon of PDE4D can be classified into three groups with clearly distinct risks. Relative to the protective group, the population attributed risk of the at-risk and wild type groups combined is estimated to be 55%. Approximately 16% of the population carries one copy of the at-risk haplotype in FIG. 12.3. They have about 1.8 times the risk of the general population for getting cardiogenic or carotid stroke. Approximately 0.8% of the population is homozygous for the at-risk haplotype and, assuming the multiplicative model, their risk is estimated to be about 3.8 times the risk of the general population. It is true that we have not yet identified or proved convincingly what is the functional variant, or variants, which are responsible for the observed effects of these haplotype groups. And, since these haplotype groups do not fully explain the linkage signal we observe in the region for all stroke patients, we certainly could not rule out, and indeed expect, that there are other variants/haplotypes within PDE4D not directly related to those we have identified that confer risk to stroke. These are likely to be rare but could have very high penetrance. We also cannot rule out the possibility that some other genes in the linkage region independent of, or in conjunction with, PDE4D confer susceptibility to stroke.

We examined whether the disease associated alleles and haplotype are related to specific stroke risk factors such as hypertension, hypercholesterolemia, diabetes, peripheral artery occlusive disease and coronary artery disease in addition to each onset of stroke and gender (Table 8). A marginally significant association to hypercholesterolemia was observed but it is clear that PDE4D's contribution to stroke is not strongly correlated with any of these known risk factors.

The PDE4D gene is a highly complex gene. By alternative splicing and use of different promoters this gene generates at least 8 different isoforms that yield functional proteins, differing from each other in their N-terminal regions. We have identified four new exons encoding the N-termini of two new isoforms PDE4D7 and PDE4D9. The disease-associated haplotype extends over the 5′exon unique to the new PDE4D7 variant and the presumed promoter region of this isoform suggesting that the functional variation may be involved in transcriptional regulation. This hypothesis is also supported by our PDE4D expression analysis that shows significant correlation between the disease associated haplotype and the level of PDE4D7 message.

The strongest association found for this PDE4D haplotype was to the two major subtypes of ischemic stroke, carotid and cardiogenic stroke, suggesting a role for this gene in the vascular biology of atherosclerosis. While there are multiple etiologies for ischemic stroke, atherosclerosis remains the most important one and it is the major pathological process for the two ischemic subtypes, carotid and cardiogenic strokes. First, it is the major cause of stenotic and occlusive lesions of the internal and common carotids that lead to carotid strokes. Second, cardiac thrombi which shed emboli to the brain most commonly occur on the background of coronary artery disease, such as following acute myocardial infarction or ischemic cardiomyopathy, and/or due to atrial fibrillation on the basis of poor compliance of ischemic ventricles (diastolic dysfunction/stiffening). Although atrial fibrillation may occur on the background of other diseases such as valvular disease, hyperthyroidism, and hypertension, in the age group that tends to suffer from stroke, ischemic heart disease remains one of the most important causes. Ischemic stroke resulting from occlusion of small penetrating arteries within the brain (small vessel occlusive disease or lacunar stroke) is generally thought to result from endothelial proliferation since atherosclerosis only occurs in larger arteries. PDE4D does not show association to small vessel stroke, consistent with its role in atherosclerosis. Carotid and cardiogenic stroke together account for the majority of ischemic stroke (note that our number for carotid is lower since we used a more stringent cutoff of stenosis).

PDE4D selectively degrades second messenger cAMP (Kong, A. et al., Nat Genet 10, 10 (2002)), which plays a central role in signal transduction and regulation of physiological responses. It is expressed in most cell types important to the pathogenesis of atherosclerosis, including vascular smooth muscle cells (VSCM), endothelial cells, monocytes, macrophages and T-lymphocytes (Houslay, M. D. and Adams, D. R., Biochem J 370, 1-18 (2003); Liu, H. and Maurice, D. H., J Biol Chem 274, 10557-65. (1999); Liu, H. et al., J Biol Chem 275, 26615-24. (2000); Baillie, G., et al., Mol Pharmacol 60, 1100-11. (2001); Jin, S. L. and Conti, M., Proc Natl Acad Sci USA 99, 7628-33. (2002)). Cyclic AMP is a key signalling-molecule in these cells (Landells, L. J. et al., Br J Pharmacol 133, 722-9 (2001); Fukumoto, S. et al., Circ Res 85, 985-91. (1999); Ogawa, S. et l., Am J Physiol 262, C546-54 (1992)). In VSMC, low cAMP levels lead to an increase in proliferation and migration that at least in part is mediated by PDE4 (Landells, L. J. et al., Br J Pharmacol 133, 722-9 (2001); Stelzner, T. J., et al., J Cell Physiol 139, 157-66 (1989); Pan, X., et al., Biochem Pharmacol 48, 827-35. (1994)). Animal models have also shown that elevation of cAMP reduces neointimal lesion formation and inhibits proliferation of SMCs after arterial injury (Palmer, D., et al., Circ Res 82, 852-61. (1998); Indolfi, C. et al., Nat Med 3, 775-9. (1997)). In monocytes and T-lymphocytes, accumulation of cAMP is generally associated with inhibition of immune functions such as proliferation and cytokine secretion (Indolfi, C. et al., J Am Coll Cardiol 36, 288-93. (2000)). It is attractive to postulate that the regulation of cAMP through absolute or relative expression of one or more PDE4D isoforms may differ in individuals susceptible to stroke; some stroke patients may have increased PDE4D activity and, consequently lower cAMP levels in any of the above cell types, leading to development of the atherosclerotic plaque and/or its instability. However, contrary to what one might expect we see decreased expression in some of the PDE4D isoforms in EBV cell lines from stroke patients. It is of interest that these isoforms are all up regulated by cAMP (Liu, H. and Maurice, D. H., J Biol Chem 274, 10557-65. (1999); Tilley, S. L., et al., J Clin Invest 108, 15-23(2001); Vicini, E. and Conti, M., Mol Endocrinol 11, 839-50 (1997)) suggesting disregulation at the level of cAMP in patients. It is therefore possible that increased activity of one or few splice variants alters the effective PDE4D enzymatic activity of the cell decreasing the cAMP levels thus altering the expression of cAMP regulated isoforms as observed in our expression study. This relative expression of PDE4D isoforms may determine the compartmental localization of PDE4D isoforms and thus the corresponding gradients of intracellular cAMP that have been recently observed (see Housley review).

In summary, we have presented association analyses (single marker and haplotype analyses) that support the notion that the PDE4D gene confers risk to ischemic stroke. Furthermore, we have observed significant disregulation of multiple PDE4D isoforms in stroke patients. We propose that this gene is involved in the pathogenesis of stroke through atherosclerosis. PDE4D is expressed in cell types important in atherosclerosis and regulates a second messenger with a central role to processes important in the pathogenesis of atherosclerosis. Inhibition of PDE4D in general or specifically one or more isoforms, by a small molecule drug or other pharmacological agent might decrease the risk of stroke in general, and especially those who are predisposed to stroke through variation in the PDE4D gene.

While this invention has been particularly shown and described with reference to preferred embodiments thereof, it will be understood by those skilled in the art that various changes in form and details may be made therein without departing from the spirit and scope of the invention as defined by the appended claims.

1. A method of diagnosing susceptibility to a stroke in an individual, comprising screening for an at-risk haplotype in the phosphodiesterase 4D gene that is more frequently present in an individual susceptible to stroke compared to a healthy individual, wherein the at-risk haplotype increases risk of stroke significantly.

2. The method of claim 1 wherein the significant increase is at least about 20%.

3. The method of claim 1 wherein the significant increase is identified as an odds ratio of at least about 1.2.

4. A method of diagnosing susceptibility to stroke in an individual, comprising screening for an at-risk haplotype in the phosphodiesterase 4D gene that is more frequently present in an individual susceptible to stroke (affected), compared to the frequency of its presence in a healthy individual (control), wherein the presence of the at-risk haplotype is indicative of a susceptibility to stroke.

5. The method of claim 4 wherein the at risk haplotype 1 is characterized by the presence of G at nucleic acid position 142780, relative to SEQ ID NO: 1 and allele 0 of microsatellite marker AC0088181-1.

6. The method of claim 4 wherein the at risk haplotype 2 is characterized by the presence of G T A A C C A C G A A C T T A T T G A A T T T G A A at nucleic acid postions: 142780, 135112, 132562, 131865, 129361, 129360, 125304, 123426, 123312, 120628, 118914, 111781, 111252, 109301, 107849, 105225, 104552, 102977, 100795, 99035, 88614, 88456, 83119, 82244, 80127, 78552, relative to SEQ ID NO: 1 and allele 0 of microsatellite marker AC0088181-1.

7. The method of claim 4 wherein the at risk haplotype 3 is characterized by the presence of A A C A A at nucleic acid positions 138806, 131865, 129361, 120628, 91470, relative to SEQ ID NO: 1.

8. The method of claim 4 wherein screening for the presence of an at-risk haplotype within or near PDE4D that significantly correlates with haplotype 1 or stroke susceptibility.

9. The method of claim 4 wherein screening for the presence of an at-risk haplotype within or near PDE4D that significantly correlates with haplotype 2 or stroke susceptibility.

10. The method of claim 4 wherein screening for the presence of an at-risk haplotype within or near PDE4D that significantly correlates with haplotype 3 or stroke susceptibility.

11. The method of claim 4 wherein screening for the presence of an at-risk haplotype in the phosphodiesterase 4D gene comprises enzymatic amplification of nucleic acid from said individual.

12. The method of claim 11 wherein the nucleic acid is DNA.

13. The method of claim 12 wherein the DNA is mammalian.

14. The method of claim 13 wherein the DNA is human.

15. The method of claim 4 wherein screening for the presence of an at-risk haplotype in the phosphodiesterase 4D gene comprises:
(a) obtaining material containing nucleic acid from the individual;
(b) amplifying said nucleic acid; and
(c) determining the presence or absence of an at-risk haplotype in said amplified nucleic acid.

16. The method of claim 15 wherein determining the presence of an at-risk haplotype is performed by electrophoretic analysis.

17. The method of claim 15 wherein determining the presence of an at-risk haplotype is performed by restriction length polymorphism analysis.

18. The method of claim 15 wherein determining the presence of an at-risk haplotype is performed by sequence analysis.

19. The method of claim 15 wherein determining the presence of an at-risk haplotype is performed by hybridization analysis.

20. A kit for diagnosing susceptibility to stroke in an individual comprising: primers for nucleic acid amplification of a region of the phosphodiesterase 4D gene comprising an at-risk haplotype.

21. The kit of claim 20 wherein the primers comprise a segment of nucleic acids of length suitable for nucleic acid amplification a single nucleotide polymorphism at nucleic acid position 142780 respectively, relative to SEQ ID NO: 1 and allele 0 of microsatellite marker AC0088181-1.

22. The kit of claim 20 wherein the primers comprise a segment of nucleic acids of length suitable for nucleic acid amplification, selected from the group consisting of: single nucleotide polymorphism or microsatellite marker at nucleic acid position 142780, 135112, 132562, 131865, 129361, 129360, 125304, 123426, 123312, 120628, 118914, 111781, 111252, 109301, 107849, 105225, 104552, 102977, 100795, 99035, 88614, 88456, 83119, 82244, 80127, 78552, relative to SEQ ID NO: 1, allele 0 of microsatellite marker AC0088181-1.
and combinations thereof.

23. The kit of claim 20 wherein the primers comprise a segment of nucleic acids of length suitable for nucleic acid amplification, selected from the group consisting of: single nucleotide polymorphism at nucleic acid position at nucleic acid position 138806, 131865, 129361, 120628, 91470, relative to SEQ ID NO: 1 and combinations thereof.

24. A method for assessing susceptibility to stroke in an individual, comprising determining PDE4D isoform expression levels in the individual compared to control, wherein a difference in isoform expression is indicative of susceptibility to stroke.

25. The method of claim 24 wherein isoform PDE4D7 and/or PDE4D9 expression is determined.

26. A method of diagnosing a susceptibility to stroke, comprising detecting an alteration in the expression or composition of a polypeptide encoded by phosphodiesterase 4D gene in a test sample, in comparison with the expression or composition of a polypeptide encoded by phosphodiesterase 4D gene in a control sample, wherein the presence of an alteration in expression or composition of the polypeptide in the test sample is indicative of a susceptibility to stroke.

27. The method of claim 26, wherein the alteration in the expression or composition of a polypeptide encoded by phosphodiesterase 4D gene comprises expression of a splicing variant polypeptide in a test sample that differs from a splicing variant polypeptide expressed in a control sample.

28. A method for preventing the occurrence of stroke in an individual in need thereof, comprising regulating a PDE4D isoform level compared to control, whereby the regulated isoform level mimics the level in a healthy individual.

29. The method of claim 28 wherein isoform level is regulated by regulating expression of the isoform using a phosphodiesterase 4D gene binding agent, a phosphodiesterase 4D gene receptor, a peptidomimetic, a fusion protein, a prodrug, an antibody or a ribozyme.

30. The method of claim 28 wherein the isoform level is controlled by genetically altering the isoform's expression level.

31. The method of claim 28 wherein the isoform level is regulated by altering the ratio of isoforms.

32. The method of claim 28 wherein isoform PDE4D7 and/or PDE4D9 is regulated.

33. A method for monitoring the effectiveness of treatment on the regulation of expression of one or more PDE4D isoforms at the RNA or protein level, or its enzymatic activity by measuring PDE4D message or protein or enzymatic activity in a sample of peripheral blood or cells derived thereof.

34. A method for predicting the effectiveness of a given therapeutic for stroke prevention or treatment in a given individual comprising screening for the presence or absence of the stroke at-risk haplotype in the phosphodiesterase 4D gene.

35. A method for predicting the effectiveness of a given therapeutic for stroke prevention or treatment in a given individual comprising screening for the expression of one or more PDE4D isoforms at the RNA or protein level, or its enzymatic activity by measuring PDE4D message or protein or enzymatic activity in a sample of peripheral blood or cells derived thereof.

36. A method of diagnosing a reduced or protective susceptibility to a stroke in an individual, comprising screening for a protective haplotype in the phosphodiesterase, 4D gene that is more frequently present in an individual compared to an individual susceptible to stroke, wherein the protective haplotype decreases the risk of stroke significantly

37. A method of claim 36 wherein the protective haplotype is characterized by the A allele at position 142780, relative to SEQ ID NO: 1 and allele-8 for microsatellitemarker AC0088181-1.
